(19) United States
(12) Patent Application Publication
(10) Pub. No.: US 2020/0123203 A1
(43) Pub. Date: Apr. 23, 2020

(54) COMPOSITIONS COMPRISING CURONS AND USES THEREOF

(71) Applicant: FLAGSHIP PIONEERING INNOVATIONS V, INC., Cambridge, MA (US)

(72) Inventors: Avak Kahvejian, Lexington, MA (US); Erica Gabrielle Weinstein, Boston, MA (US); Nicholas McCartney Plugis, Boston, MA (US); Kevin James Lebo, Weymouth, MA (US); Fernando Martin Diaz, Boston, MA (US); Dhananjay Maniklal Nawandar, Brookline, MA (US)

(73) Assignee: FLAGSHIP PIONEERING INNOVATIONS V, INC., Cambridge, MA (US)

(21) Appl. No.: 16/622,146

(22) PCT Filed: Jun. 13, 2018
(86) PCT No.: PCT/US2018/037379

§ 371 Date: Dec. 12, 2019

Related U.S. Application Data
(60) Provisional application No. 62/518,898, filed on Jun. 13, 2017.
(60) Provisional application No. 62/597,387, filed on Dec. 11, 2017.
(60) Provisional application No. 62/676,730, filed on May 25, 2018.

(51) Int. Cl. C07K 14/005; C12N 15/86; C12N 7/00; C12N 15/113

(52) U.S. Cl. CPC C07K 14/005; C12N 15/86; C12N 7/00; C12N 15/1136; C12N 2750/00022; C12N 2310/141; C12N 2320/32; C12N 2750/00021; C12N 2750/00043

(57) ABSTRACT

This invention relates generally to pharmaceutical compositions and preparations of curons and uses thereof.

### RELATED APPLICATIONS

This application claims priority to U.S. Ser. No. 62/518,898 filed Jun. 13, 2017, U.S. Ser. No. 62/597,387 filed Dec. 11, 2017, and U.S. Ser. No. 62/676,730 filed May 25, 2018, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 13, 2018, is named V2057-7000WO_SL.txt and is 1,066,292 bytes in size.

### BACKGROUND

Existing viral systems for delivering therapeutic agents utilize viruses that can be associated with diseases or disorders, and can be highly immunogenic. There exists a need in the art for improved delivery vehicles that are substantially non-immunogenic and non-pathogenic.

### SUMMARY

The present disclosure provides a curon, e.g., a synthetic curon, that can be used as a delivery vehicle, e.g., for delivering a therapeutic agent to a eukaryotic cell. In some embodiments, a curon comprises a particle comprising a genetic element encapsulated in a proteinaceous exterior, which is capable of introducing the genetic element into a cell (e.g., a human cell). In some instances, the genetic element comprises a payload, e.g., it encodes an exogenous effector (e.g., a nucleic acid effector, such as a non-coding RNA, or a polypeptide effector, e.g., a protein) that is expressed in the cell. For example, the curon can deliver an exogenous effector into a cell by contacting the cell and introducing a genetic element encoding the exogenous effector into the cell, such that the exogenous effector is made or expressed by the cell. The exogenous effector can, in some instances, modulate a function of the cell or modulate an activity or level of a target molecule in the cell. For example, the exogenous effector may decrease viability of a cancer cell (e.g., as described in Example 22) or decrease levels of a target protein, e.g., interferon, in the cell (e.g., as described in Examples 3 and 4). In another example, the exogenous effector may be a protein expressed by the cell (e.g., as described in Example 9).

A synthetic curon has at least one structural difference compared to a wild-type virus, e.g., a deletion, insertion, substitution, enzymatic modification, relative to a wild-type virus. Generally, synthetic curons include an exogenous genetic element enclosed within a proteinaceous exterior, which can be used as substantially non-immunogenic vehicles for delivering the genetic element, or an effector (e.g., an exogenous effector or an endogenous effector) encoded therein (e.g., a polypeptide or nucleic acid effector), into eukaryotic cells. Curons can be used for treatment of diseases and disorders, e.g., by delivering a therapeutic agent to a desired cell or tissue. The genetic element of a synthetic curon of the present disclosure can be a circular single-stranded DNA molecule, and generally includes a protein binding sequence that binds to the proteinaceous exterior, or a polypeptide attached thereto, which may facilitate enclosure of the genetic element within the proteinaceous exterior and/or enrichment of the genetic element, relative to other nucleic acids, within the proteinaceous exterior.

In an aspect, the invention features a synthetic curon comprising (i) a genetic element comprising a promoter element, a sequence encoding an exogenous effector, (e.g., a payload), and a protein binding sequence (e.g., an exterior protein binding sequence, e.g., a packaging signal). In some embodiments, the genetic element is a single-stranded DNA. Alternatively or in combination, the genetic element has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior. In some embodiments, the genetic element is enclosed within the proteinaceous exterior. In some embodiments, the synthetic curon is capable of delivering the genetic element into a eukaryotic cell.

In an aspect, the invention features a synthetic curon comprising: (i) a genetic element comprising a promoter element and a sequence encoding an exogenous effector (e.g., a payload), and a protein binding sequence (e.g., an exterior protein binding sequence); and (ii) a proteinaceous exterior; wherein the genetic element is enclosed within the proteinaceous exterior; and wherein the synthetic curon is capable of delivering the genetic element into a eukaryotic cell. In some embodiments, the genetic element comprises a nucleic acid sequence (e.g., a nucleic acid sequence of between 300-4000 nucleotides, e.g., between 300-3500 nucleotides, between 300-3000 nucleotides, between 300-2500 nucleotides, between 300-2000 nucleotides, between 300-1500 nucleotides) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Torque Teno virus (TTV), Torque Teno mini virus (TTMV), or TTMDV sequence, e.g., a wild-type Anellovirus sequence as listed in any of Tables 1, 3, 5, 7, 9, 11, or 13). In some embodiments, the genetic element comprises a nucleic acid sequence (e.g., a nucleic acid sequence of at least 300 nucleotides, 500 nucleotides, 1000 nucleotides, 1500 nucleotides, 2000 nucleotides, 2500 nucleotides, 3000 nucleotides or more) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Torque Teno virus (TTV), Torque Teno mini virus (TTMV), or TTMDV sequence, e.g., a wild-type Anellovirus sequence as listed in any of Tables 1, 3, 5, 7, 9, 11, or 13).

In an aspect, the invention features a method of treating a disease or disorder in a subject, the method comprising administering to the subject a curon, e.g., a synthetic curon, e.g., as described herein. In some embodiments, the curon comprises: (i) a genetic element comprising a promoter element and a sequence encoding an effector, e.g., a payload, and an exterior protein binding sequence. In some embodiments, the genetic element is a single-stranded DNA, and wherein the genetic element is circular and/or integrates at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the curon is capable of delivering the genetic element into a eukaryotic cell.

In an aspect, the invention features a method of delivering a payload to a cell, tissue or subject, the method comprising administering to the subject a curon, e.g., a synthetic curon, e.g., as described herein, wherein the curon comprises a nucleic acid sequence encoding the payload. In some embodiments, the curon comprises: (i) a genetic element comprising a promoter element and a sequence encoding an effector, e.g., a payload, and an exterior protein binding sequence. In some embodiments, the genetic element is a single-stranded DNA, and wherein the genetic element is circular and/or integrates at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the curon is capable of delivering the genetic element into a eukaryotic cell. In embodiments, the payload is a nucleic acid. In embodiments, the payload is a protein.

In an aspect, the invention features a method of delivering a synthetic curon to a cell, comprising contacting the synthetic curon described herein, e.g., of any of the aspects herein (e.g., the preceding aspects) with a cell, e.g., a eukaryotic cell, e.g., a mammalian cell.

In an aspect, the invention features a pharmaceutical composition comprising a curon (e.g., a synthetic curon) as described herein. In embodiments, the pharmaceutical composition further comprises a pharmaceutically acceptable carrier or excipient. In embodiments, the pharmaceutical composition comprises a dose comprising about 10⁵-10¹⁴ genome equivalents of the curon per kilogram.

In an aspect, the invention features a nucleic acid molecule comprising a genetic element comprising a promoter element and a sequence encoding an effector, e.g., a payload, and an exterior protein binding sequence. In embodiments, the genetic element is a single-stranded DNA, and wherein the genetic element is circular and/or integrates at a frequency of less than about 0.001%, 0.005%, 0.01%, 0.05%, 0.1%, 0.5%, 1%, 1.5%, or 2% of the genetic element that enters the cell. In embodiments, the effector does not originate from TTV and is not an SV40-miR-S1. In embodiments, the nucleic acid molecule does not comprise the polynucleotide sequence of TTMV-LY. In embodiments, the promoter element is capable of directing expression of the effector in a eukaryotic cell.

In an aspect, the invention features a genetic element comprising one, two, or three of: (i) a promoter element and a sequence encoding an effector, e.g., a payload; wherein the effector is exogenous relative to a wild-type Anellovirus sequence; (ii) at least 72 contiguous nucleotides (e.g., at least 72, 73, 74, 75, 76, 77, 78, 79, 80, 90, 100, or 150 nucleotides) having at least 75% (e.g., at least 75, 76, 77, 78, 79, 80, 90, 91, 92, 93, 94, 95, 96, 97, 98, 99, or 100%) sequence identity to a wild-type Anellovirus sequence; or at least 100 (e.g., at least 300, 500, 1000, 1500) contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type Anellovirus sequence; and (iii) a protein binding sequence, e.g., an exterior protein binding sequence, and wherein the nucleic acid construct is a single-stranded DNA; and wherein the nucleic acid construct is circular and/or integrates at a frequency of less than about 0.001%, 0.005%, 0.01%, 0.05%, 0.1%, 0.5%, 1%, 1.5%, or 2% of the genetic element that enters the cell.

In an aspect, the invention features a method of manufacturing a synthetic curon composition, comprising:

a) providing a host cell comprising, e.g., expressing one or more components (e.g., all of the components) of a curon, e.g., a synthetic curon, e.g., as described herein;

b) producing a preparation of curons from the host cell, wherein the synthetic curons of the preparation comprise a proteinaceous exterior and a genetic element comprising a promoter element, a sequence encoding an exogenous effector, (e.g., a payload), and a protein binding sequence (e.g., an exterior protein binding sequence, e.g., a packaging signal), thereby making a preparation of synthetic curon; and

c) formulating the preparation of synthetic curons, e.g., as a pharmaceutical composition suitable for administration to a subject.

In an aspect, the invention features a method of manufacturing a synthetic curon composition, comprising: a) providing a plurality of synthetic curon described herein, or a pharmaceutical composition described herein; and b) formulating the synthetic curons, e.g., as a pharmaceutical composition suitable for administration to a subject.

In an aspect, the invention features a method of making a host cell, e.g., a first host cell or a producer cell (e.g., as shown in FIG. 12), e.g., a population of first host cells, comprising a synthetic curon, the method comprising introducing a genetic element, e.g., as described herein, to a host cell and culturing the host cell under conditions suitable for production of the synthetic curon. In embodiments, the method further comprises introducing a helper, e.g., a helper virus, to the host cell. In embodiments, the introducing comprises transfection (e.g., chemical transfection) or electroporation of the host cell with the synthetic curon.

In an aspect, the invention features a method of making a synthetic curon, comprising providing a host cell, e.g., a first host cell or producer cell (e.g., as shown in FIG. 12), comprising a synthetic curon, e.g., as described herein, and purifying the curon from the host cell. In some embodiments, the method further comprises, prior to the providing step, contacting the host cell with a synthetic curon, e.g., as described herein, and incubating the host cell under conditions suitable for production of the synthetic curon. In embodiments, the host cell is the first host cell or producer cell described in the above method of making a host cell. In embodiments, purifying the curon from the host cell comprises lysing the host cell.

In some embodiments, the method further comprises a second step of contacting the synthetic curon produced by the first host cell or producer cell with a second host cell, e.g., a permissive cell (e.g., as shown in FIG. 12), e.g., a population of second host cells. In some embodiments, the method further comprises incubating the second host cell inder conditions suitable for production of the synthetic curon. In some embodiments, the method further comprises purifying a synthetic curon from the second host cell, e.g., thereby producing a curon seed population. In embodiments, at least about 2-100-fold more of the synthetic curon is produced from the population of second host cells than from the population of first host cells. In embodiments, purifying the curon from the second host cell comprises lysing the second host cell.

In some embodiments, the method further comprises a second step of contacting the synthetic curon produced by the second host cell with a third host cell, e.g., permissive cells (e.g., as shown in FIG. 12), e.g., a population of third host cells. In some embodiments, the method further comprises incubating the third host cell inder conditions suitable for production of the synthetic curon. In some embodiments, the method further comprises purifying a synthetic curon from the third host cell, e.g., thereby producing a curon stock population. In embodiments, purifying the curon from the third host cell comprises lysing the third host cell. In embodiments, at least about 2-100-fold more of the synthetic curon is produced from the population of third host cells than from the population of second host cells.

In some embodiments, the method further comprises evaluating one or more synthetic curons from the curon seed population or the curon stock population for one or more quality control parameters, e.g., purity, titer, potency (e.g., in genomic equivalents per curon particle), and/or the nucleic acid sequence, e.g., from the genetic element comprised by the synthetic curon. In some embodiments, the evaluated nucleic acid sequence comprises the nucleic acid sequence encoding an exogenous effector.

In an aspect, the invention comprises evaluating one or more synthetic curons, e.g., from a curon seed population or a curon stock population, for one or more quality control parameters, e.g., purity, titer, potency, and/or the nucleic acid sequence, e.g., from the genetic element comprised by the synthetic curon. In some embodiments, the evaluated nucleic acid sequence comprises the nucleic acid sequence encoding an exogenous effector.

In an aspect, the invention features a reaction mixture comprising a synthetic curon described herein and a helper virus, wherein the helper virus comprises a polynucleotide, e.g., a polynucleotide encoding an exterior protein, (e.g., an exterior protein capable of binding to the exterior protein binding sequence and, optionally, a lipid envelope), a polynucleotide encoding a replication protein (e.g., a polymerase), or any combination thereof.

In some embodiments, a curon (e.g., a synthetic curon) is isolated, e.g., isolated from a host cell and/or isolated from other constituents in a solution (e.g., a supernatant). In some embodiments, a curon (e.g., a synthetic curon) is purified, e.g., from a solution (e.g., a supernatant). In some embodiments, a curon is enriched in a solution relative to other constituents in the solution.

In some embodiments of any of the aforesaid curons, compositions or methods, the genetic element comprises a minimal curon genome, e.g., as identified according to the method described in Example 9. In some embodiments, the minimal curon genome comprises a minimal Anellovirus genome sufficient for replication of the curon (e.g., in a host cell). In embodiments, the minimal curon genome comprises a TTV-tth8 nucleic acid sequence, e.g., a TTV-tth8 nucleic acid sequence shown in Table 5, having deletions of at least 10%, 20%, 30%, 40%, 50%, 60%, 70%, 80%, 90%, 95%, 99%, or 100% of nucleotides 3436-3707 of the TTV-tth8 nucleic acid sequence. In embodiments, the minimal curon genome comprises a TTMV-LY2 nucleic acid sequence, e.g., a TTMV-LY2 nucleic acid sequence shown in Table 11, having deletions of at least 10%, 20%, 30%, 40%, 50%, 60%, 70%, 80%, 90%, 95%, 99%, or 100% of nucleotides 574-1371, 1432-2210, 574-2210, and/or 2610-2809 of the TTMV-LY2 nucleic acid sequence. In embodiments, the minimal curon genome is a minimal curon genome capable of self-replication and/or self-amplification. In embodiments, the minimal curon genome is a minimal curon genome capable of replicating or being amplified in the presence of a helper, e.g., a helper virus.

Additional features of any of the aforesaid curons, compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### Enumerated Embodiments

1. A synthetic curon comprising:

(i) a genetic element comprising a promoter element, a nucleic acid sequence (e.g., a DNA sequence) encoding an exogenous effector, (e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and

(ii) a proteinaceous exterior;

wherein the genetic element is enclosed within the proteinaceous exterior; and

wherein the synthetic curon is capable of delivering the genetic element into a eukaryotic cell.

2. A synthetic curon comprising:

(i) a genetic element comprising a promoter element and a nucleic acid sequence (e.g., a DNA sequence) encoding an exogenous effector (e.g., a payload), and a protein binding sequence (e.g., an exterior protein binding sequence),

wherein the genetic element has at least 75% (e.g., at least 75, 76, 77, 78, 79, 80, 85, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence, e.g., as listed in any of Tables 1, 3, 5, 7, 9, 11, or 13); and

(ii) a proteinaceous exterior;

wherein the genetic element is enclosed within the proteinaceous exterior; and

wherein the synthetic curon is capable of delivering the genetic element into a eukaryotic cell.

3. A synthetic curon comprising:

(i) a genetic element comprising a promoter element and a nucleic acid sequence (e.g., a DNA sequence) encoding an effector (e.g., an exogenous effector or endogenous effector, e.g., endogenous miRNA), and a protein binding sequence (e.g., an exterior protein binding sequence),

wherein the genetic element has at least 75% (e.g., at least 75, 76, 77, 78, 79, 80, 85, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence, e.g., as listed in any of Tables 1, 3, 5, 7, 9, 11, or 13); and

wherein the genetic element is not a naturally occurring sequence (e.g., comprises a deletion, substitution, or insertion relative to a wild-type Anellovirus sequence (e.g., a wild-type Torque Teno virus (TTV), Torque Teno mini virus (TTMV), or TTMDV sequence, e.g., a wild-type Anellovirus sequence, e.g., as listed in any of Tables 1, 3, 5, 7, 9, 11, or 13);

(ii) a proteinaceous exterior;

wherein the genetic element is enclosed within the proteinaceous exterior; and

wherein the synthetic curon is capable of delivering the genetic element into a eukaryotic cell.

4. A synthetic curon comprising:

(i) a genetic element comprising a promoter element and a nucleic acid sequence (e.g., a DNA sequence) encoding an exogenous effector (e.g., a payload), and a protein binding sequence (e.g., an exterior protein binding sequence),

wherein the protein binding sequence has at least 75% (e.g., at least 75, 76, 77, 78, 79, 80, 85, 90, 91, 92, 93, 94, 95, 96, 97, 98, 99, or 100%) sequence identity to the Consensus 5′ UTR sequence shown in Table 16-1, or to the Consensus GC-rich sequence shown in Table 16-2, or both of the Consensus 5′ UTR sequence shown in Table 16-1 and to the Consensus GC-rich sequence shown in Table 16-2; and

(ii) a proteinaceous exterior;

wherein the genetic element is enclosed within the proteinaceous exterior; and

wherein the synthetic curon is capable of delivering the genetic element into a eukaryotic cell.

5. A synthetic curon comprising:

(i) a genetic element comprising a promoter element and a nucleic acid sequence encoding an exogenous effector, and a protein binding sequence, wherein the genetic element comprises one or both of:
(a) a sequence having at least 85% sequence identity to the Anellovirus 5′ UTR conserved domain nucleotide sequence of nucleotides 323-393 of the nucleic acid sequence of Table 11, or
(b) a sequence having at least 85% sequence identity to the Anellovirus GC-rich region of nucleotides 2868-2929 of the nucleic acid sequence of Table 11; and

(ii) a proteinaceous exterior; wherein the genetic element is enclosed within the proteinaceous exterior; and

wherein the synthetic curon is capable of delivering the genetic element into a eukaryotic cell.

6. A synthetic curon comprising:

(i) a genetic element comprising a promoter element and a nucleic acid sequence encoding an exogenous effector, and a protein binding sequence, wherein the genetic element comprises one or both of:
(a) a sequence having at least 85% sequence identity to the Anellovirus 5′ UTR conserved domain of the nucleic acid sequence of Table 1, 3, 5, 7, 9 or 13; or
(b) a sequence having at least 85% sequence identity to the Anellovirus GC-rich region of the nucleic acid sequence of Table 1, 3, 5, 7, 9 or 13; and

(ii) a proteinaceous exterior; wherein the genetic element is enclosed within the proteinaceous exterior; and

wherein the synthetic curon is capable of delivering the genetic element into a eukaryotic cell.

7. The synthetic curon of any of the preceding embodiments, wherein the promoter element comprises an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc).

8. The synthetic curon of any of the preceding embodiments, wherein the promoter element comprises a TATA box.

9. The synthetic curon of any of the preceding embodiments, wherein the promoter element is endogenous to a wild-type Anellovirus, e.g., a wild-type Anellovirus sequence as listed in any of Tables 1, 3, 5, 6, 9, 11, or 13.

10. The synthetic curon of any of embodiments 1-8, wherein the promoter element is exogenous to wild-type Anellovirus.

11. The synthetic curon of any of the preceding embodiments, wherein the exogenous effector encodes a therapeutic agent, e.g., a therapeutic peptide or polypeptide or a therapeutic nucleic acid.

12. The synthetic curon of any of the preceding embodiments, wherein the exogenous effector comprises a regulatory nucleic acid, e.g., an miRNA, siRNA, mRNA, lncRNA, RNA, DNA, an antisense RNA, gRNA; a fluorescent tag or marker, an antigen, a peptide, a synthetic or analog peptide from a naturally-bioactive peptide, an agonist or antagonist peptide, an anti-microbial peptide, a pore-forming peptide, a bicyclic peptide, a targeting or cytotoxic peptide, a degradation or self-destruction peptide, a small molecule, an immune effector (e.g., influences susceptibility to an immune response/signal), a death protein (e.g., an inducer of apoptosis or necrosis), a non-lytic inhibitor of a tumor (e.g., an inhibitor of an oncoprotein), an epigenetic modifying agent, an epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand, an antibody, a receptor, or a CRISPR system or component.

13. The synthetic curon of any of the preceding embodiments, wherein the exogenous effector comprises a miRNA.

14. The synthetic curon of any of the preceding embodiments, wherein the effector, e.g., miRNA, targets a host gene, e.g., modulates expression of the gene, e.g., increases or decreases expression of the gene.

15. The synthetic curon of any of the preceding embodiments, wherein the exogenous effector comprises an miRNA, and decreases expression of a host gene.

16. The synthetic curon of any of the preceding embodiments, wherein the exogenous effector comprises a nucleic acid sequence about 20-200, 30-180, 40-160, 50-140, or 60-120 nucleotides in length.

17. The synthetic curon of any of the preceding embodiments, wherein the nucleic acid sequence encoding the exogenous effector is about 20-200, 30-180, 40-160, 50-140, or 60-120 nucleotides in length.

18. The synthetic curon of any of the preceding embodiments, wherein the sequence encoding the exogenous effector has a size of at least about 100 nucleotides.

19. The synthetic curon of any of the preceding embodiments, wherein the sequence encoding the exogenous effector has a size of about 100 to about 5000 nucleotides.

20. The synthetic curon of any of the preceding embodiments, wherein the sequence encoding the exogenous effector has a size of about 100-200, 200-300, 300-400, 400-500, 500-600, 600-700, 700-800, 800-900, 900-1000, 1000-1500, or 1500-2000 nucleotides.

21. The synthetic curon of any of the preceding embodiments, wherein the sequence encoding the exogenous effector is situated at, within, or adjacent to (e.g., 5′ or 3′ to) one or more of the ORF1 locus (e.g., at the C-terminus of the ORF1 locus), the miRNA locus, the 5′ noncoding region upstream of the TATA box, the 5′ UTR, the 3′ noncoding region downstream of the poly-A region, or a noncoding region upstream of the GC-rich region of the genetic element.

22. The synthetic curon of embodiment 21, wherein the sequence encoding the exogenous effector is located between the poly-A region and the GC-rich region of the genetic element.

23. The synthetic curon of any of the preceding embodiments, which comprises (e.g., in the proteinaceous exterior) one or more of an amino acid sequence chosen from ORF2, ORF2/2, ORF2/3, ORF1, ORF1/1, or ORF1/2 of Table 12, or an amino acid sequence having at least 85% sequence identity thereto.

24. The synthetic curon of any of the preceding embodiments, which comprises (e.g., in the proteinaceous exterior) one or more of an amino acid sequence chosen from ORF2, ORF2/2, ORF2/3, ORF2t/3, ORF1, ORF1/1, or ORF1/2 of any of Tables 2, 4, 6, 8, 10, or 14, or an amino acid sequence having at least 85% sequence identity thereto.

25. The synthetic curon of any of the preceding embodiments, wherein the protein binding sequence comprises a nucleic acid sequence having at least 75% (e.g., at least 75, 76, 77, 78, 79, 80, 90, 91, 92, 93, 94, 95, 96, 97, 98, 99, or 100%) sequence identity to the 5′ UTR conserved domain or the GC-rich domain of a wild-type Anellovirus, e.g., a wild-type Anellovirus sequence as listed in any of Tables 1, 3, 5, 6, 9, 11, 13, A, or B.

26. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the Consensus 5′ UTR nucleic acid sequence shown in Table 16-1.

27. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the exemplary TTV 5′ UTR nucleic acid sequence shown in Table 16-1.

28. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-CT30F 5′ UTR nucleic acid sequence shown in Table 16-1.

29. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-HD23a 5′ UTR nucleic acid sequence shown in Table 16-1.

30. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-JA20 5′ UTR nucleic acid sequence shown in Table 16-1.

31. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-TJN02 5′ UTR nucleic acid sequence shown in Table 16-1.

32. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-tth8 5′ UTR nucleic acid sequence shown in Table 16-1.

33. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the Consensus GC-rich region shown in Table 16-2.

34. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the exemplary TTV GC-rich region shown in Table 16-2.

35. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-CT30F GC-rich region shown in Table 16-2.

36. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-HD23a GC-rich region shown in Table 16-2.

37. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-JA20 GC-rich region shown in Table 16-2.

38. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-TJN02 GC-rich region shown in Table 16-2.

39. The synthetic curon of any of the preceding embodiments, wherein the genetic element, e.g., protein binding sequence of the genetic element, comprises least about 75% (e.g., at least 75%, 80%, 85%, 90%, 95%, 96%, 97%, 98%, 99%, or 100%) identity to the TTV-tth8 GC-rich region shown in Table 16-2.

40. The synthetic curon of any of the preceding embodiments, wherein at least 60% (e.g., at least 60%, 65%, 70%, 75%, 80%, 85%, 90%, 95%, or 100%) of the protein binding sequence consists of G or C.

41. The synthetic curon of any of the preceding embodiments, wherein the genetic element comprises a sequence of at least 80, 90, 100, 110, 120, 130, or 140 nucleotides in length, which consists of G or C at least 70% (e.g., at least 70%, 75%, 80%, 85%, 90%, 95%, 96%, 97%, 98%, 99%, or 100%) or about 70-100%, 75-95%, 80-95%, 85-95%, or 85-90% of the positions.

42. The synthetic curon of any of the preceding embodiments, wherein the genetic element comprises a sequence having at least 85% sequence identity to the Anellovirus 5′ UTR conserved domain nucleotide sequence of nucleotides 1-393 of the nucleic acid sequence of Table 11 and a sequence having at least 85% sequence identity to the Anellovirus GC-rich region of nucleotides 2868-2929 of the nucleic acid sequence of Table 11.

43. The synthetic curon of any of the preceding embodiments, wherein the protein binding sequence is capable of binding to an exterior protein, e.g., a capsid protein, e.g., an Anellovirus capsid protein, e.g., a capsid protein comprising an amino acid sequence having at least 75% (e.g., at least 75, 76, 77, 78, 79, 80, 90, 91, 92, 93, 94, 95, 96, 97, 98, 99, or 100%) sequence identity to any of the sequences listed in Table 1-14, 16, or 18.

44. The synthetic curon of any of the preceding embodiments, wherein the genetic element comprises at least 75% identity to the nucleotide sequence of Table 11.

45. The synthetic curon of any of the preceding embodiments, wherein the protein binding sequence binds an arginine-rich region of the proteinaceous exterior.

46. The synthetic curon of any of the preceding embodiments, wherein the proteinaceous exterior comprises an exterior protein capable of specifically binding to the protein binding sequence.

47. The synthetic curon of embodiment 46, wherein the exterior protein comprises a capsid protein e.g., an Anellovirus capsid protein, e.g., a capsid protein comprising an amino acid sequence having at least 75% (e.g., at least 75, 76, 77, 78, 79, 80, 90, 91, 92, 93, 94, 95, 96, 97, 98, 99, or 100%) sequence identity to any of the sequences listed in any of Tables 1-14, 16, or 18 or an amino acid sequence encoded by any of the sequences listed in Table 1-14, 15, 17, or 19, or a fragment thereof.

48. The synthetic curon of any of the preceding embodiments, wherein the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

49. The synthetic curon of any of the preceding embodiments, wherein the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or substantially non-pathogenic in a host.

50. The synthetic curon of any of the preceding embodiments, wherein the proteinaceous exterior comprises at least one functional domain that provides one or more functions, e.g., species and/or tissue and/or cell selectivity, genetic element binding and/or packaging, immune evasion (substantial non-immunogenicity and/or tolerance), pharmacokinetics, endocytosis and/or cell attachment, nuclear entry, intracellular modulation and localization, exocytosis modulation, propagation, and nucleic acid protection.

51. The synthetic curon of any of the preceding embodiments, wherein the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

52. The synthetic curon of any of the preceding embodiments, wherein the genetic element is single-stranded.

53. The synthetic curon of any of the preceding embodiments, wherein the genetic element is circular.

54. The synthetic curon of any of the preceding embodiments, wherein the genetic element is DNA.

55. The synthetic curon of any of the preceding embodiments, wherein the genetic element is a negative strand DNA.

56. The synthetic curon of any of the preceding embodiments, wherein the genetic element comprises an episome.

57. The synthetic curon of any of the preceding embodiments, wherein the synthetic curon has a lipid content of less than 10%, 5%, 2%, or 1% by weight, e.g., does not comprise a lipid bilayer.

58. The synthetic curon of any of the preceding embodiments, wherein the synthetic curon is resistant to degradation by a detergent (e.g., a mild detergent, e.g., a biliary salt, e.g., sodium deoxycholate) relative to a viral particle comprising an external lipid bilayer, e.g., a retrovirus.

59. The synthetic curon of embodiment 58, wherein at least about 50% (e.g., at least about 50%, 60%, 70%, 80%, 85%, 90%, 95%, 96%, 97%, 98%, 99%, 99.5%, or 99.9%) of the synthetic curon is not degraded after incubation the detergent (e.g., 0.5% by weight of the detergent) for 30 minutes at 37° C.

60. The synthetic curon of any of the preceding embodiments, wherein the genetic element has at least 75% (e.g., at least 75, 76, 77, 78, 79, 80, 90, 91, 92, 93, 94, 95, 96, 97, 98, 99, or 100%) sequence identity to a wild-type Circoviridae sequence or a wild-type Anellovirus sequence, e.g., a wild-type Torque Teno virus (TTV), Torque Teno mini virus (TTMV), or TTMDV sequence, e.g., a sequence as listed in any of Tables 1, 3, 5, 7, 9, 11, or 13.

61. The synthetic curon of embodiment 60, wherein the genetic element comprises a deletion of at least one element, e.g., an element as listed in any of Tables 1, 3, 5, 7, 9, 11, or 13, relative to a wild-type Anellovirus sequence, e.g., a wild-type TTV sequence or a wild-type TTMV sequence.

62. The synthetic curon of embodiment 61, wherein the genetic element comprises a deletion comprising a nucleic acid sequence corresponding to nucleotides 3436-3607 of a TTV-tth8 sequence, e.g., the nucleic acid sequence shown in Table 5.

63. The synthetic curon of embodiment 61, wherein the genetic element comprises a deletion comprising a nucleic acid sequence corresponding to nucleotides 574-1371 and/or nucleotides 1432-2210 of a TTMV-LY2 sequence, e.g., the nucleic acid sequence shown in Table 11.

64. The synthetic curon of embodiment 61 or 62, wherein the genetic element comprises a deletion comprising a nucleic acid sequence corresponding to nucleotides 1372-1431 of a TTMV-LY2 sequence, e.g., the nucleic acid sequence shown in Table 11.

65. The synthetic curon of embodiment 61, 63, or 64, wherein the genetic element comprises a deletion comprising a nucleic acid sequence corresponding to nucleotides 2610-2809 of a TTMV-LY2 sequence, e.g., the nucleic acid sequence shown in Table 11.

66. The synthetic curon of any of the preceding embodiments, wherein the genetic element comprises at least 72 nucleotides (e.g., at least 73, 74, 75, etc. nt, optionally less than the full length of the genome) of a wild-type Anellovirus sequence, e.g., a wild-type Torque Teno virus (TTV), Torque Teno mini virus (TTMV), or TTMDV sequence, e.g., a sequence as listed in any of Tables 1, 3, 5, 7, 9, 11, or 13.

67. The synthetic curon of any of the preceding embodiments, wherein the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), a sequence that encodes a therapeutic mRNA or protein, and a sequence that encodes a cytolytic/cytotoxic RNA or protein.

68. The synthetic curon of any of the preceding embodiments, wherein the synthetic curon further comprises a second genetic element, e.g., a second genetic element enclosed within the proteinaceous exterior.

69. The synthetic curon of embodiment 68, wherein the second genetic element comprises a protein binding sequence, e.g., an exterior protein binding sequence, e.g., a packaging signal, e.g., a 5′ UTR conserved domain or GC-rich region, e.g., as described herein.

70. The synthetic curon of any of the preceding embodiments, wherein the synthetic curon does not detectably infect bacterial cells, e.g., infects less than 1%, 0.5%, 0.1%, or 0.01% of bacterial cells.

71. The synthetic curon of any of the preceding embodiments, wherein the synthetic curon is capable of infecting mammalian cells, e.g., human cells, e.g., immune cells, liver cells, epithelial cells, e.g., in vitro.

72. The synthetic curon of any of the preceding embodiments, wherein the genetic element integrates at a frequency of less than 10%, 8%, 6%, 4%, 3%, 2%, 1%, 0.5%, 0.2%, 0.1% of the curons that enters the cell, e.g., wherein the synthetic curon is non-integrating.

73. The synthetic curon of any of the preceding embodiments, wherein the genetic element is capable of replicating, e.g., capable of generating at least 10², 2×10², 5×10², 10³, 2×10³, 5×10³, or 10⁴ genomic equivalents of the genetic element per cell, e.g., as measured by a quantitative PCR assay.

74. The synthetic curon of any of the preceding embodiments, wherein the genetic element is capable of replicating, e.g., capable of generating at least 10², 2×10², 5×10², 10³, 2×10³, 5×10³, or 10⁴ more genomic equivalents of the genetic element in a cell, e.g., as measured by a quantitative PCR assay, than were present in the synthetic curon prior to delivery of the genetic element into the cell.

75. The synthetic curon of any of the preceding embodiments, wherein the genetic element is not capable of replicating, e.g., wherein the genetic element is altered at a replication origin or lacks a replication origin.

76. The synthetic curon of any of the preceding embodiments, wherein the genetic element is not capable of self-replicating, e.g., capable of being replicated without being integrated into a host cell genome.

77. The synthetic curon of any of the preceding embodiments, wherein the synthetic curon is substantially non-pathogenic, e.g., does not induce a detectable deleterious symptom in a subject (e.g., elevated cell death or toxicity, e.g., relative to a subject not exposed to the curon).

78. The synthetic curon of any of the preceding embodiments, wherein the synthetic curon is substantially non-immunogenic, e.g., does not induce a detectable and/or unwanted immune response, e.g., as detected according to the method described in Example 4.

79. The synthetic curon of embodiment 78, wherein the substantially non-immunogenic curon has an efficacy in a subject that is a least about 10%, 20%, 30%, 40%, 50%, 60%, 70%, 80%, 90%, 95%, or 100% of the efficacy in a reference subject lacking an immune response.

80. The synthetic curon of embodiment 78 or 79, wherein the immune response comprises one or more of an antibody specific to the curon; a cellular response (e.g., an immune effector cell (e.g., T cell- or NK cell) response) against the curon or cells comprising the curon; or macrophage engulfment of the curon or cells comprising the curon.

81. The synthetic curon of any of the preceding embodiments, wherein the synthetic curon is less immunogenic than an AAV, elicits an immune response below that detected for a comparable quantity of AAV, e.g., as measured by an assay described herein, induces an antibody prevalence of less than 70% (e.g., less than about 60%, 50%, 40%, 30%, 20%, or 10% antibody prevalence) as measured by an assay described herein, or is substantially non-immunogenic.

82. The synthetic curon of any of the preceding embodiments, wherein a population of at least 1000 of the synthetic curons is capable of delivering at least 100 copies of the genetic element into one or more of the eukaryotic cells.

83. The synthetic curon of any of the preceding embodiments, wherein a population of the synthetic curons is capable of delivering the genetic element into at least 10%, 20%, 30%, 40%, 50%, 60%, 70%, 80%, 90%, 95%, 99%, or more of a population of the eukaryotic cells.

84. The synthetic curon of any of the preceding embodiments, wherein a population of the synthetic curons is capable of delivering at least 1, 2, 5, 10, 20, 50, 100, 200, 500, 1000, 2000, 5000, 8,000, 1×10⁴, 1×10⁵, 1×10⁶, 1×10⁷ or greater copies of the genetic element per cell to a population of the eukaryotic cells.

85. The synthetic curon of any of the preceding embodiments, wherein a population of the synthetic curons is capable of delivering 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of the genetic element per cell to a population of the eukaryotic cells.

86. The synthetic curon of any of the preceding embodiments, wherein the synthetic curon is present after at least two passages.

87. The synthetic curon of any of the preceding embodiments, wherein the synthetic curon was produced by a process comprising at least two passages.

88. The synthetic curon of any of the preceding embodiments, wherein the synthetic curon selectively delivers the exogenous effector to a desired cell type, tissue, or organ (e.g., photoreceptors in the retina, epithelial linings, or pancreas).

89. The synthetic curon of any of the preceding embodiments, wherein the synthetic curon shows greater selectivity in vitro for an embryonic kidney cell line (e.g., HEK293T) than a lung epithelial carcinoma cell line (e.g., A549).

90. The synthetic curon of any of the preceding embodiments, wherein the synthetic curon is present at higher levels in (e.g., preferentially accumulates in) a desired organ or tissue relative to other organs or tissues.

91. The synthetic curon of embodiment 90, wherein the desired organ or tissue comprises bone marrow, blood, heart, GI, or skin.

92. The synthetic curon of any of the preceding embodiments, wherein the eukaryotic cell is a mammalian cell, e.g., a human cell.

93. The synthetic curon of any of the preceding embodiments, wherein the synthetic curon, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into the cell.

94. The synthetic curon of any of the preceding embodiments, wherein the synthetic curon is produced in the cell pellet and the supernatant at least about 10⁸-fold (e.g., about 10⁵-fold, 10⁶-fold, 10⁷-fold, 10⁸-fold, 10⁹-fold, or 10¹⁰-fold) genomic equivalents/mL, e.g., relative to the quantity of the synthetic curon used to infect the cells, after 3-4 days post infection, e.g., using an infectivity assay, e.g., an assay according to Example 7.

95. A composition comprising the synthetic curon of any of the preceding embodiments.

96. A pharmaceutical composition comprising the synthetic curon of any of the preceding embodiments, and a pharmaceutically acceptable carrier or excipient.

97. The composition or pharmaceutical composition of embodiment 95 or 96, which comprises at least 50%, 60%, 70%, 80%, 90%, 95%, 96%, 97%, 98%, 99%, or more curons, e.g., synthetic curons.

98. The composition or pharmaceutical composition of any of embodiments 95-97, which comprises at least 10³, 10⁴, 10⁵, 10⁶, 10⁷, 10⁸, or 10⁹ synthetic curons.

99. A pharmaceutical composition comprising
a) at least 10³, 10⁴, 10⁵, 10⁶, 10⁷, 10⁸, or 10⁹ curons (e.g., synthetic curons described herein) comprising:
   (i) a genetic element described herein, e.g., a genetic element comprising a promoter element, a nucleic acid sequence (e.g., a DNA sequence) encoding an exogenous effector, (e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and
   (ii) a proteinaceous exterior,
   wherein the genetic element is enclosed within the proteinaceous exterior; and
   wherein the synthetic curon is capable of delivering the genetic element into a eukaryotic cell;
b) a pharmaceutical excipient, and, optionally,
c) less than a pre-determined amount of: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted curons, free viral capsid protein, adventitious agents, and/or aggregates.

100. A pharmaceutical composition comprising
a) at least 10³, 10⁴, 10⁵, 10⁶, 10⁷, 10⁸, or 10⁹ curons (e.g., synthetic curons described herein) comprising:
   (i) a genetic element described herein, e.g., a genetic element comprising a promoter element and a nucleic acid sequence (e.g., a DNA sequence) encoding an exogenous effector (e.g., a payload), and a protein binding sequence (e.g., an exterior protein binding sequence),
   wherein the genetic element has at least 75% (e.g., at least 75, 76, 77, 78, 79, 80, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence, e.g., as listed in any of Tables 1, 3, 5, 7, 9, 11, or 13); and
   (ii) a proteinaceous exterior;
   wherein the genetic element is enclosed within the proteinaceous exterior; and
   wherein the synthetic curon is capable of delivering the genetic element into a eukaryotic cell
b) a pharmaceutical excipient, and, optionally,
c) less than a pre-determined amount of: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted curons, free viral capsid protein, adventitious agents, and/or aggregates.

101. The composition or pharmaceutical composition of any of embodiments 95-100, having one or more of the following characteristics:

a) the pharmaceutical composition meets a pharmaceutical or good manufacturing practices (GMP) standard;

b) the pharmaceutical composition was made according to good manufacturing practices (GMP);

c) the pharmaceutical composition has a pathogen level below a predetermined reference value, e.g., is substantially free of pathogens;

d) the pharmaceutical composition has a contaminant level below a predetermined reference value, e.g., is substantially free of contaminants;

e) the pharmaceutical composition has a predetermined level of non-infectious particles or a predetermined ratio of particles:infectious units (e.g., <300:1, <200:1, <100:1, or <50:1), or

f) the pharmaceutical composition has low immunogenicity or is substantially non-immunogenic, e.g., as described herein.

102. The composition or pharmaceutical composition of any of embodiments 95-101, wherein the pharmaceutical composition has a contaminant level below a predetermined reference value, e.g., is substantially free of contaminants.

103. The composition or pharmaceutical composition of embodiment 102, wherein the contaminant is selected from the group consisting of: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted curons (e.g., a curon other than the desired curon, e.g., a synthetic curon as described herein), free viral capsid protein, adventitious agents, and aggregates.

104. The composition or pharmaceutical composition of embodiment 103, wherein the contaminant is host cell DNA and the threshold amount is about 500 ng of host cell DNA per dose of the pharmaceutical composition.

105. The composition or pharmaceutical composition of any of embodiments 95-104, wherein the pharmaceutical composition comprises less than 10% (e.g., less than about 10%, 5%, 4%, 3%, 2%, 1%, 0.5%, or 0.1%) contaminant by weight.

106. Use of the synthetic curon, composition, or pharmaceutical composition of any of the preceding embodiments for treating a disease or disorder in a subject.

107. The use of embodiment 106, wherein the disease or disorder is chosen from an immune disorder, an interferonopathy (e.g., Type I interferonopathy), infectious disease, inflammatory disorder, autoimmune condition, cancer (e.g., a solid tumor, e.g., lung cancer), and a gastrointestinal disorder.

108. The synthetic curon, composition, or pharmaceutical composition of any of the preceding embodiments for use in treating a disease or disorder in a subject.

109. A method of treating a disease or disorder in a subject, the method comprising administering a synthetic curon of any of the preceding embodiments or the pharmaceutical composition of any of embodiments 95-105 to the subject.

110. The method of embodiment 109, wherein the disease or disorder is chosen from an immune disorder, an interferonopathy (e.g., Type I interferonopathy), infectious disease, inflammatory disorder, autoimmune condition, cancer (e.g., a solid tumor, e.g., lung cancer), and a gastrointestinal disorder.

111. A method of modulating, e.g., enhancing, a biological function in a subject, the method comprising administering a synthetic curon of any of the preceding embodiments or the pharmaceutical composition of any of embodiments 95-105 to the subject.

112. A method of treating a disease or disorder in a subject, the method comprising administering to the subject a curon, e.g., synthetic curon, comprising:
(i) a genetic element comprising a promoter element and a sequence encoding an effector, e.g., a payload, and an exterior protein binding sequence;
wherein the genetic element is a single-stranded DNA, and wherein the genetic element is circular and/or integrates at a frequency of less than about 0.001%, 0.005%, 0.01%, 0.05%, 0.1%, 0.5%, 1%, 1.5%, or 2% of the genetic element that enters a cell; and

(ii) a proteinaceous exterior;

wherein the genetic element is enclosed within the proteinaceous exterior; and

wherein the curon, e.g., synthetic curon, is capable of delivering the genetic element into a eukaryotic cell.

113. The method of embodiment 112, wherein the disease or disorder is chosen from an immune disorder, an interferonopathy (e.g., Type I interferonopathy), infectious disease, inflammatory disorder, autoimmune condition, cancer (e.g., a solid tumor, e.g., lung cancer), and a gastrointestinal disorder.

114. The method of any of embodiments 109-113, wherein the effector is not an SV40-miR-S1, e.g., wherein the effector is a protein-encoding payload.

115. The method of any of embodiments 109-114, wherein the curon does not comprise an exogenous effector.

116. The method of any of embodiments 109-115, wherein the curon comprises a wild-type Circovirus or a wild-type Anellovirus, e.g., TTV or TTMV.

117. The method of any of embodiments 109-116, wherein the administration of the curon, e.g., synthetic curon, results in delivery of the genetic element into at least 10%, 20%, 30%, 40%, 50%, 60%, 70%, 80%, 90%, 95%, 99%, or more of a population of target cells in the subject.

118. The method of any of embodiments 109-117, wherein the administration of the curon, e.g., synthetic curon, results in delivery of the exogenous effector into at least 10%, 20%, 30%, 40%, 50%, 60%, 70%, 80%, 90%, 95%, 99%, or more of a population of target cells in the subject.

119. The method of embodiment 117 or 118, wherein the target cells comprise mammalian cells, e.g., human cells, e.g., immune cells, liver cells, lung epithelial cells, e.g., in vitro.

120. The method of any of embodiments 117-119, wherein the target cells are present in the liver or lung.

121. The method of any of embodiments 117-120, wherein the target cells into which the genetic element is delivered each receive at least 10, 50, 100, 500, 1000, 10,000, 50,000, 100,000, or more copies of the genetic element.

122. The method of any of embodiments 109-121, wherein the effector comprises a miRNA and wherein the miRNA reduces the level of a target protein or RNA in a cell or in a population of cells, e.g., into which the curon is delivered, e.g., by at least 10%, 20%, 30%, 40%, or 50%.

123. A method of delivering a synthetic curon to a cell, comprising contacting the synthetic curon of any of the preceding embodiments with a cell, e.g., a eukaryotic cell, e.g., a mammalian cell.

124. The method of embodiment 123, further comprising contacting a helper virus with the cell, wherein the helper virus comprises a polynucleotide, e.g., a polynucleotide encoding an exterior protein, e.g., an exterior protein capable of binding to the exterior protein binding sequence and, optionally, a lipid envelope.

125. The method of embodiment 124, wherein the helper virus is contacted with the cell prior to, concurrently with, or after contacting the synthetic curon with the cell.

126. The method of embodiment 123, further comprising contacting a helper polynucleotide with the cell.

127. The method of embodiment 126, wherein the helper polynucleotide comprises a sequence polynucleotide encoding an exterior protein, e.g., an exterior protein capable of binding to the exterior protein binding sequence and a lipid envelope.

128. The method of embodiment 126, wherein the helper polynucleotide is an RNA (e.g., mRNA), DNA, plasmid, viral polynucleotide, or any combination thereof.

129. The method of any of embodiments 126-128, wherein the helper polynucleotide is contacted with the cell prior to, concurrently with, or after contacting the synthetic curon with the cell.

130. The method of any of embodiments 123-129, further comprising contacting a helper protein with the cell.

131. The method of embodiment 130, wherein the helper protein comprises a viral replication protein or a capsid protein.

132. A host cell comprising the synthetic curon of any of the preceding embodiments.

133. A nucleic acid molecule comprising a promoter element, a sequence encoding an effector (e.g., a payload), and an exterior protein binding sequence,

wherein the nucleic acid molecule is a single-stranded DNA, and wherein the nucleic acid molecule is circular and/or integrates at a frequency of less than about 0.001%, 0.005%, 0.01%, 0.05%, 0.1%, 0.5%, 1%, 1.5%, or 2% of the nucleic acid molecule that enters a cell;

wherein the effector does not originate from TTV and is not an SV40-miR-S1;

wherein the nucleic acid molecule does not comprise the polynucleotide sequence of TTMV-LY;

wherein the promoter element is capable of directing expression of the effector in a eukaryotic cell.

134. A nucleic acid molecule comprising a promoter element and a nucleic acid sequence encoding an exogenous effector, and a protein binding sequence, wherein the genetic element comprises one or both of:
(a) a sequence having at least 85% sequence identity to the Anellovirus 5′ UTR conserved domain nucleotide sequence of nucleotides 323-393 of the nucleic acid sequence of Table 11, or
(b) a sequence having at least 85% sequence identity to the Anellovirus GC-rich region of nucleotides 2868-2929 of the nucleic acid sequence of Table 11.

135. A nucleic acid molecule comprising a promoter element and a nucleic acid sequence encoding an exogenous effector, and a protein binding sequence, wherein the genetic element comprises one or both of:
(a) a sequence having at least 85% sequence identity to the Anellovirus 5′ UTR conserved domain of the nucleic acid sequence of Table 1, 3, 5, 7, 9 or 13; or
(b) a sequence having at least 85% sequence identity to the Anellovirus GC-rich region of the nucleic acid sequence of Table 1, 3, 5, 7, 9 or 13.

136. A genetic element comprising:

(i) a promoter element and a sequence encoding an effector, e.g., a payload, wherein the effector is exogenous relative to a wild-type Anellovirus sequence;

(ii) at least 72 contiguous nucleotides (e.g., at least 72, 73, 74, 75, 76, 77, 78, 79, 80, 90, 100, or 150 nucleotides) having at least 75% sequence identity to a wild-type Anellovirus sequence; or at least 100 contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type Anellovirus sequence; and

(iii) a protein binding sequence, e.g., an exterior protein binding sequence, and

wherein the nucleic acid construct is a single-stranded DNA; and

wherein the nucleic acid construct is circular and/or integrates at a frequency of less than about 0.001%, 0.005%, 0.01%, 0.05%, 0.1%, 0.5%, 1%, 1.5%, or 2% of the genetic element that enters a cell.

137. A method of manufacturing a synthetic curon composition, comprising:

a) providing a host cell comprising one or more nucleic acid molecules encoding the components of a synthetic curon, e.g., a synthetic curon described herein, wherein the synthetic curon comprises a proteinaceous exterior and a genetic element, e.g., a genetic element comprising a promoter element, a sequence encoding an exogenous effector, (e.g., a payload), and a protein binding sequence (e.g., an exterior protein binding sequence, e.g., a packaging signal);

b) producing a synthetic curon from the host cell, thereby making a synthetic curon; and

c) formulating the synthetic curons, e.g., as a pharmaceutical composition suitable for administration to a subject.

138. A method of manufacturing a synthetic curon composition, comprising:
a) providing a plurality of synthetic curons according to any of the preceding embodiments, or a composition or pharmaceutical composition of any of embodiments 95-105;
b) optionally evaluating the plurality for one or more of: a contaminant described herein, an optical density measurement (e.g., OD 260), particle number (e.g., by HPLC), infectivity (e.g., particle:infectious unit ratio); and
c) formulating the plurality of synthetic curons, e.g., as a pharmaceutical composition suitable for administration to a subject, e.g., if one or more of the parameters of (b) meet a specified threshold.

139. The method of embodiment 138, wherein the synthetic curon composition comprises at least 10⁵, 10⁶, 10⁷, 10⁸, 10⁹, 10¹⁰, 10¹¹, 10¹², 10¹³, 10¹⁴, or 10¹⁵ synthetic curons.

140. The method of embodiment 138 or 139, wherein the synthetic curon composition comprises at least 10 ml, 20 ml, 50 ml, 100 ml, 200 ml, 500 ml, 1 L, 2 L, 5 L, 10 L, 20 L, or 50 L.

141. A reaction mixture comprising the synthetic curon of any of the preceding embodiments and a helper virus, wherein the helper virus comprises a polynucleotide, e.g., a polynucleotide encoding an exterior protein, e.g., an exterior protein capable of binding to the exterior protein binding sequence and, optionally, a lipid envelope.

142. A reaction mixture comprising the synthetic curon of any of the preceding embodiments and a second nucleic acid sequence encoding one or more of an amino acid sequence chosen from ORF2, ORF2/2, ORF2/3, ORF1, ORF1/1, or ORF1/2 of Table 12, or an amino acid sequence having at least 85% sequence identity thereto.

143. A reaction mixture comprising the synthetic curon of any of the preceding embodiments and a second nucleic acid sequence encoding one or more of an amino acid sequence chosen from ORF2, ORF2/2, ORF2/3, ORF2t/3, ORF1, ORF1/1, or ORF1/2 of any of Tables 2, 4, 6, 8, 10, or 14, or an amino acid sequence having at least 85% sequence identity thereto.

144. The reaction mixture of embodiment 142 or 143, wherein the second nucleic acid sequence is part of the genetic element.

145. The reaction mixture of embodiment 144, wherein the second nucleic acid sequence is not part of the genetic element, e.g., the second nucleic acid sequence is comprised by a helper cell or helper virus.

146. A synthetic curon comprising:
a genetic element comprising (i) a sequence encoding a non-pathogenic exterior protein, (ii) an exterior protein binding sequence that binds the genetic element to the non-pathogenic exterior protein, and (iii) a sequence encoding an effector, e.g., a regulatory nucleic acid; and
a proteinaceous exterior that is associated with, e.g., envelops or encloses, the genetic element.

147. A pharmaceutical composition comprising
a) a curon comprising:
   a genetic element comprising (i) a sequence encoding a non-pathogenic exterior protein, (ii) an exterior protein binding sequence that binds the genetic element to the non-pathogenic exterior protein, and (iii) a sequence encoding an effector, e.g., a regulatory nucleic acid; and
   a proteinaceous exterior that is associated with, e.g., envelops or encloses, the genetic element; and
b) a pharmaceutical excipient.

148. A pharmaceutical composition comprising
a) at least 10³, 10⁴, 10⁵, 10⁶, 10⁷, 10⁸, or 10⁹ curons (e.g., synthetic curons described herein) comprising:
   a genetic element comprising (i) a sequence encoding a non-pathogenic exterior protein, (ii) an exterior protein binding sequence that binds the genetic element to the non-pathogenic exterior protein, and (iii) a sequence encoding an effector, e.g., a regulatory nucleic acid; and
   a proteinaceous exterior that is associated with, e.g., envelops or encloses, the genetic element;
b) a pharmaceutical excipient, and, optionally,
c) less than a pre-determined amount of: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted curons, free viral capsid protein, adventitious agents, and/or aggregates.

149. The curon or composition of any one of the previous embodiments, further comprising at least one of the following characteristics: the genetic element is a single-stranded DNA; the genetic element is circular; the curon is non-integrating; the curon has a sequence, structure, and/or function based on an anellovirus or other non-pathogenic virus, and the curon is non-pathogenic.

150. The curon or composition of any one of the previous embodiments, wherein the proteinaceous exterior comprises the non-pathogenic exterior protein.

151. The curon or composition of any one of the previous embodiments, wherein the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

152. The curon or composition of any one of the previous embodiments, wherein the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is non-immunogenic or non-pathogenic in a host.

153. The curon or composition of any one of the previous embodiments, wherein the sequence encoding the non-pathogenic exterior protein comprise a sequence at least 70%, 80%, 85%, 90%, 95%, 96%, 97%, 98%, 99% identical to one or more sequences or a fragment thereof listed in Table 15.

154. The curon or composition of any one of the previous embodiments, wherein the non-pathogenic exterior protein comprises a sequence at least 70%, 80%, 85%, 90%, 95%, 96%, 97%, 98%, 99% identical to one or more sequences or a fragment thereof listed in Table 16 or Table 17.

155. The curon or composition of any one of the previous embodiments, wherein the non-pathogenic exterior protein comprises at least one functional domain that provides one or more functions, e.g., species and/or tissue and/or cell tropism, viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection.

156. The curon or composition of any one of the previous embodiments, wherein the effector comprises a regulatory nucleic acid, e.g., an miRNA, siRNA, mRNA, lncRNA, RNA, DNA, an antisense RNA, gRNA; a therapeutic, e.g., fluorescent tag or marker, antigen, peptide therapeutic, synthetic or analog peptide from naturally-bioactive peptide, agonist or antagonist peptide, anti-microbial peptide, pore-forming peptide, a bicyclic peptide, a targeting or cytotoxic peptide, a degradation or self-destruction peptide, and degradation or self-destruction peptides, small molecule, immune effector (e.g., influences susceptibility to an immune response/signal), a death protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component.

157. The curon or composition of any one of the previous embodiments, wherein the effector comprises a sequence at least 70%, 80%, 85%, 90%, 95%, 96%, 97%, 98%, 99% identical to one or more miRNA sequences listed in Table 18.

158. The curon or composition of the previous embodiment, wherein the effector, e.g., miRNA, targets a host gene, e.g., modulates expression of the gene.

159. The curon or composition of the previous embodiment, wherein the miRNA, e.g., has at least 70%, 80%, 85%, 90%, 95%, 96%, 97%, 98%, 99% identity to one or more sequences listed in Table 16.

160. The curon or composition of any one of the previous embodiments, wherein the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), a sequence that encodes a therapeutic mRNA or protein, and a sequence that encodes a cytolytic/cytotoxic RNA or protein.

161. The curon or composition of any one of the previous embodiments, wherein the genetic element has one or more of the following characteristics: is non-integrating with a host cell's genome, is an episomal nucleic acid, is a single stranded DNA, is about 1 to 10 kb, exists within the nucleus of the cell, is capable of being bound by endogenous proteins, and produces a microRNA that targets host genes.

162. The curon or composition of any one of the previous embodiments, wherein the genetic element comprises at least one viral sequence or at least 70%, 80%, 85%, 90%, 95%, 96%, 97%, 98%, 99% identity to one or more sequences or a fragment thereof listed in Table 19 or Table 20.

163. The curon or composition of the previous embodiment, wherein the viral sequence is from at least one of a single stranded DNA virus (e.g., Anellovirus, Bidnavirus, Circovirus, Geminivirus, Genomovirus, Inovirus, Microvirus, Nanovirus, Parvovirus, and Spiravirus), a double stranded DNA virus (e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus), a RNA virus (e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus).

164. The curon or composition of the previous embodiment, wherein the viral sequence is from one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus.

165. The curon or composition of any one of the previous embodiments, wherein the protein binding sequence interacts with the arginine-rich region of the proteinaceous exterior.

166. The curon or composition of any one of the previous embodiments, wherein the curon is capable of replicating in a mammalian cell, e.g., human cell.

167. The curon or composition of the previous embodiment, wherein the curon is non-pathogenic and/or non-integrating in a host cell.

168. The curon or composition of any one of the previous embodiments, wherein the curon is non-immunogenic in a host.

169. The curon or composition of any one of the previous embodiments, wherein the curon inhibits/enhances one or more viral properties, e.g., selectivity, e.g., infectivity, e.g., immunosuppression/activation, in a host or host cell.

170. The curon or composition of the previous embodiment, wherein the curon is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more).

171. The composition of any one of the previous embodiments further comprising at least one virus or vector comprising a genome of the virus, e.g., a variant of the curon, e.g., a commensal/native virus.

172. The composition of any one of the previous embodiments further comprising a heterologous moiety, at least one small molecule, antibody, polypeptide, nucleic acid, targeting agent, imaging agent, nanoparticle, and a combination thereof.

173. A vector comprising a genetic element comprising (i) a sequence encoding a non-pathogenic exterior protein, (ii) an exterior protein binding sequence that binds the genetic element to the non-pathogenic exterior protein, and (iii) a sequence encoding an effector, e.g., a regulatory nucleic acid.

174. The vector of the previous embodiment, wherein the genetic element fails to integrate with a host cell's genome.

175. The vector of any one of the previous embodiments, wherein the genetic element is capable of replicating in a mammalian cell, e.g., human cell.

176. The vector of any one of the previous embodiments further comprising an exogenous nucleic acid sequence, e.g., selected to modulate expression of a gene, e.g., a human gene.

177. A pharmaceutical composition comprising the vector of any one of the previous embodiments and a pharmaceutical excipient.

178. The composition of the previous embodiment, wherein the vector is non-pathogenic and/or non-integrating in a host cell.

179. The composition of any one of the previous embodiments, wherein the vector is non-immunogenic in a host.

180. The composition of the previous embodiment, wherein the vector is in an amount sufficient to modulate (phenotype, virus levels, gene expression, compete with other viruses, disease state, etc. at least about 5%, 10%, 15%, 20%, 25%, 30%, 35%, 40%, 45%, 50%, or more).

181. The composition of any one of the previous embodiments further comprising at least one virus or vector comprising a genome of the virus, e.g., a variant of the curon, a commensal/native virus, a helper virus, a non-anellovirus.

182. The composition of any one of the previous embodiments further comprising a heterologous moiety, at least one small molecule, antibody, polypeptide, nucleic acid, targeting agent, imaging agent, nanoparticle, and a combination thereof.

183. A method of producing, propagating, and harvesting the curon of any one of the previous embodiments.

184. A method of designing and making the vector of any one of the previous embodiments.

185. A method of administering to a subject an effective amount of the composition of any one of the previous embodiments.

186. A method of identifying dysvirosis in a subject comprising:
analyzing genetic information from a sample obtained from a subject in need thereof, wherein viral genetic information is isolated from the subject's genetic information and other microorganisms;
comparing the viral genetic information to a reference, e.g., a control, a healthy subject; and
identifying dysvirosis in the subject if comparison of the viral genetic information yields an imbalance or irregular ratio of viral genetic information in the subject.

187. A method of delivering a nucleic acid or protein payload to a target cell, tissue or subject, the method comprising contacting the target cell, tissue or subject with a nucleic acid composition that comprises (a) a first DNA sequence derived from a virus wherein the first DNA sequence is sufficient to enable the production of a particle capable of infecting the target cell, tissue or subject and (a) a second DNA sequence encoding the nucleic acid or protein payload, the improvement comprising:

the first DNA sequence comprises at least 500 (at least 600, 700, 800, 900, 1000, 1200, 1400, 1500, 1600, 1800, 2000) nucleotides having at least 80% (at least 85%, 90%, 95%, 97%, 99%, 100%) sequence identity to a corresponding sequence listed in any of Tables 1, 3, 5, 7, 9, 11, or 13, or

the first DNA sequence encodes a sequence having at least 80% (at least 85%, 90%, 95%, 97%, 99%, 100%) sequence identity to an ORF listed in Table 2, 4, 6, 8, 10, 12, or 14, or

the first DNA sequence comprises a sequence having at least 90% (at least 95%, 97%, 99%, 100%) sequence identity to a consensus sequence listed in Table 14-1.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1A is an illustration showing percent sequence similarity of amino acid regions of capsid protein sequences.

FIG. 1B is an illustration showing percent sequence similarity of capsid protein sequences.

FIG. 2 is an illustration showing one embodiment of a curon.

FIG. 3 depicts a schematic of a kanamycin vector encoding the LY1 strain of TTMiniV (“Curon 1”).

FIG. 4 depicts a schematic of a kanamycin vector encoding the LY2 strain of TTMiniV (“Curon 2”).

FIG. 5 depicts transfection efficiency of synthetic curons in 293T and A549 cells.

FIGS. 6A and 6B depict quantitative PCR results that illustrate successful infection of 293T cells by synthetic curons.

FIGS. 7A and 7B depict quantitative PCR results that illustrate successful infection of A549 cells by synthetic curons.

FIGS. 8A and 8B depict quantitative PCR results that illustrate successful infection of Raji cells by synthetic curons.

FIGS. 9A and 9B depict quantitative PCR results that illustrate successful infection of Jurkat cells by synthetic curons.

FIGS. 10A and 10B depict quantitative PCR results that illustrate successful infection of Chang cells by synthetic curons.

FIGS. 11A-11B are a series of graphs showing luciferase expression from cells transfected or infected with TTMV-LY2Δ574-1371,Δ1432-2210,2610::nLuc. Luminescence was observed in infected cells, indicating successful replication and packaging.

FIG. 11C is a diagram depicting the phylogenetic tree of alphatorquevirus (Torque Teno Virus; TTV), with clades highlighted. At least 100 Anellovirus strains are represented, divided into five clades. Exemplary sequences from each of the five clades is provided herein, e.g., in Tables 1-14. Top box=clade 1; Top middle box=clade 2; Middle box=clade 3, Lower middle box=clade 4; Bottom box=clade 5.

FIG. 12 is a schematic showing an exemplary workflow for production of curons (e.g., replication-competent or replication-deficient curons as described herein).

FIG. 13 is a graph showing primer specificity for primer sets designed for quantification of TTV and TTMV genomic equivalents. Quantitative PCR based on SYBR green chemistry shows one distinct peak for each of the amplification products using TTMV or TTV specific primer sets, as indicated, on plasmids encoding the respective genomes.

FIG. 14 is a series of graphs showing PCR efficiencies in the quantification of TTV genome equivalents by qPCR. Increasing concentrations of primers and a fixed concentration of hydrolysis probe (250 nM) were used with two different commercial qPCR master mixes. Efficiencies of 90-110% resulted in minimal error propagation during quantification.

FIG. 15 is a graph showing an exemplary amplification plot for linear amplification of TTMV (Target 1) or TTV (Target 2) over a 7 log 10 of genome equivalent concentrations. Genome equivalents were quantified over 7 10-fold dilutions with high PCR efficiencies and linearity (R² TTMV: 0.996; R² TTV: 0.997).

FIGS. 16A-16B are a series of graphs showing quantification of TTMV genome equivalents in a curon stock. (A) Amplification plot of two stocks, each diluted 1:10 and run in duplicate. (B) The same two samples as shown in panel A, here shown in the context of the linear range. Shown are the upper and lower limits in the two representative samples. PCR Efficiency: 99.58%, R²: 0988.

FIGS. 17A and 17B are a series of graphs showing the functional effects of a synthetic curon comprising an exogenous miRNA, miR-625. (A) Impact on cell viability of non-small cell lung cancer (NSCLC) cells when infected with curons expressing miR-625 in three different NSCLC cell lines (A549 cells, NCI-H40 cells, and SW900 cells). (B) Impact of curons expressing miR-625 on expression of a YFP reporter by HEK293T cells.

FIG. 17C is a graph showing quantification of p65 immunoblot analysis normalized to total protein for SW900 cells, either contacted with the indicated curons or left untreated.

FIG. 18 is a diagram showing pairwise identity for alignments of viral DNA sequences within the five alphatorquevirus clades. DNA sequences for viruses from each TTV clade were aligned. Pairwise percent identity across a 50-bp sliding window is shown along the length of the alignments for each clade. Average pairwise identity is indicated.

FIG. 19 is a diagram showing pairwise identity for alignments of representative sequences from each alphatorquevirus clade. DNA sequences for TTV-CT30F, TTV-TJN02, TTV-tth8, TTV-JA20, and TTV-HD23a were aligned. Pairwise percent identity across a 50-bp sliding window is shown along the length of the alignment. Brackets above indicate non-coding and coding regions with pairwise identities are indicated. Brackets below indicate regions of high sequence conservation.

FIG. 20 is a diagram showing pairwise identity for amino acid alignments for putative proteins across the five alphatorquevirus clades. Amino acid sequences for putative proteins from TTV-CT30F, TTV-TJN02, TTV-tth8, TTV-JA20, and TTV-HD23a were aligned. Pairwise percent identity across a 50-aa sliding window is shown along the length of each alignment. Pairwise identity for both open reading frame DNA sequence and protein amino acid sequence is indicated.

FIG. 21 is a diagram showing that a domain within the 5′ UTR is highly conserved across the five alphatorquevirus clades. The 71-bp 5′UTR conserved domain sequences for each representative alphatorquevirus were aligned. The sequence has 96.6% pairwise identity between the five clades. The sequences shown in FIG. 21 (SEQ ID NOS 703-708, respectively, in order of appearance) are also listed, e.g., in Table 16-1 herein.

FIG. 22 is a diagram showing an alignment of the GC-rich domains from the five alphatorquevirus clades. Each anellovirus has a region downstream of the ORFs with greater than 70% GC content. Shown is an alignment of the GC-rich regions from TTV-CT30F, TTV-TJN02, TTV-tth8, TTV-JA20, and TTV-HD23a. The regions vary in length, but where they align, they show a 81.8% pairwise identity. The sequences shown in FIG. 22 (SEQ ID NOS 709-714, respectively, in order of appearance) are also listed, e.g., in Table 16-2 herein.

### DETAILED DESCRIPTION OF CERTAIN EMBODIMENTS

### Definitions

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ” or “use of a compound, composition, product, etc in the manufacture of a medicament, pharmaceutical composition, veterinary composition, diagnostic composition, etc. for . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

As used herein, the term “curon” refers to a vehicle comprising a genetic element, e.g., an episome, e.g., circular DNA, enclosed in a proteinaceous exterior. A “synthetic curon,” as used herein, generally refers to a curon that is not naturally occurring, e.g., has a sequence that is modified relative to a wild-type virus (e.g., a wild-type Anellovirus as described herein). In some embodiments, the synthetic curon is engineered or recombinant, e.g., comprises a genetic element that comprises a modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior, e.g., prior to entry into a host cell. In some embodiments, the curon is purified, e.g., it is separated from its original source and/or substantially free (>50%, >60%, >70%, >80%, >90%) of other components.

As used herein, a nucleic acid “encoding” refers to a nucleic acid sequence encoding an amino acid sequence or a functional polynucleotide (e.g., a non-coding RNA, e.g., an siRNA or miRNA).

As used herein, the term “dysvirosis” refers to a dysregulation of the virome in a subject.

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

As used herein, the term “genetic element” refers to a nucleic acid sequence, generally in a curon. It is understood that the genetic element can be produced as naked DNA and optionally further assembled into a proteinaceous exterior. It is also understood that a curon can insert its genetic element into a cell, resulting in the genetic element being present in the cell and the proteinaceous exterior not necessarily entering the cell.

As used herein, a “substantially non-pathogenic” organism, particle, or component, refers to an organism, particle (e.g., a virus or a curon, e.g., as described herein), or component thereof that does not cause or induce a detectable disease or pathogenic condition, e.g., in a host organism, e.g., a mammal, e.g., a human. In some embodiments, administration of a curon to a subject can result in minor reactions or side effects that are acceptable as part of standard of care.

As used herein, the term “non-pathogenic” refers to an organism or component thereof that does not cause or induce a detectable disease or pathogenic condition, e.g., in a host organism, e.g., a mammal, e.g., a human.

As used herein, a “substantially non-integrating” genetic element refers to a genetic element, e.g., a genetic element in a virus or curon, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization.

As used herein, a “substantially non-immunogenic” organism, particle, or component, refers to an organism, particle (e.g., a virus or curon, e.g., as described herein), or component thereof, that does not cause or induce an undesired or untargeted immune response, e.g., in a host tissue or organism (e.g., a mammal, e.g., a human). In embodiments, the substantially non-immunogenic organism, particle, or component does not produce a detectable immune response. In embodiments, the substantially non-immunogenic curon does not produce a detectable immune response against a protein comprising an amino acid sequence or encoded by a nucleic acid sequence shown in any of Tables 1-14. In embodiments, an immune response (e.g., an undesired or untargeted immune response) is detected by assaying antibody presence or level (e.g., presence or level of an anti-curon antibody, e.g., presence or level of an antibody against a synthetic curon as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies against an Anellovirus or a curon based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

As used herein, the term “proteinaceous exterior” refers to an exterior component that is predominantly protein.

As used herein, the term “regulatory nucleic acid” refers to a nucleic acid sequence that modifies expression, e.g., transcription and/or translation, of a DNA sequence that encodes an expression product. In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “replication protein” refers to a protein, e.g., a viral protein, that is utilized during infection, viral genome replication/expression, viral protein synthesis, and/or assembly of the viral components.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder); and supportive treatment (treatment employed to supplement another therapy).

As used herein, the term “virome” refers to viruses in a particular environment, e.g., a part of a body, e.g., in an organism, e.g. in a cell, e.g. in a tissue.

This invention relates generally to curons, e.g., synthetic curons, and uses thereof. The present disclosure provides synthetic curons, compositions comprising synthetic curons, and methods of making or using synthetic curons. Synthetic curons are generally useful as delivery vehicles, e.g., for delivering a therapeutic agent to a eukaryotic cell. Generally, a synthetic curon will include a genetic element comprising an exogenous nucleic acid sequence (e.g., encoding an exogenous effector) enclosed within a proteinaceous exterior. Synthetic curons can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells.

### Curon

In some aspects, the invention described herein comprises compositions and methods of using and making a synthetic curon. In some embodiments, a curon comprises a genetic element (e.g., circular DNA, e.g., single stranded DNA), which comprise at least one exogenous element relative to the remainder of the genetic element and/or the proteinaceous exterior (e.g., an exogenous element encoding an effector, e.g., as described herein). A curon may be a delivery vehicle (e.g., a substantially non-pathogenic delivery vehicle) for a payload into a host, e.g., a human. In some embodiments, the curon is capable of replicating in a eukaryotic cell, e.g., a mammalian cell, e.g., a human cell. In some embodiments, the curon is substantially non-pathogenic and/or substantially non-integrating in the mammalian (e.g., human) cell. In some embodiments, the curon is substantially non-immunogenic in a mammal, e.g., a human. In some embodiments, the curon has a sequence, structure, and/or function that is based on an Anellovirus (e.g., an Anellovirus as described, e.g., an Anellovirus comprising a nucleic acid or polypeptide comprising a sequence as shown in any of Tables 1-14) or other substantially non-pathogenic virus, e.g., a symbiotic virus, commensal virus, native virus. Generally, an Anellovirus-based curon comprises at least one element exogenous to that Anellovirus, e.g., an exogenous effector or a nucleic acid sequence encoding an exogenous effector disposed within a genetic element of the curon. In some embodiments, the curon is replication-deficient. In some embodiments, the curon is replication-competent.

In an aspect, the invention includes a synthetic curon comprising (i) a genetic element comprising a promoter element, a sequence encoding an exogenous effector, (e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the synthetic curon is capable of delivering the genetic element into a eukaryotic cell.

In some embodiments of the synthetic curon described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the synthetic curons administered to a subject will integrate into the genome of one or more host cells in the subject. In some embodiments, the genetic elements of a population of synthetic curons, e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes a synthetic curon comprising: (i) a genetic element comprising a promoter element and a sequence encoding an exogenous effector (e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence as listed in any of Tables 1, 3, 5, 7, 9, 11, or 13); and (ii) a proteinaceous exterior; wherein the genetic element is enclosed within the proteinaceous exterior; and wherein the synthetic curon is capable of delivering the genetic element into a eukaryotic cell.

In one aspect, the invention includes a synthetic curon comprising:

a) a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and

b) a proteinaceous exterior that is associated with, e.g., envelops or encloses, the genetic element.

In some embodiments, the curon includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the curon modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In embodiments, the promoter element comprises a TATA box. In embodiments, the promoter element is endogenous to a wild-type Anellovirus, e.g., as described herein.

In some embodiments, the genetic element comprises one or more of the following characteristics: single-stranded, circular, negative strand, and/or DNA. In embodiments, the genetic element comprises an episome. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

The curons, compositions comprising curons, methods using such curons, etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaceous exteriors, for example a capsid disclosed in Arch Virol (2007) 152: 1961-1975, to produce curons which can then be used to deliver an exogenous effector to cells (e.g., animal cells, e.g., human cells or non-human animal cells such as pig or mouse cells). In embodiments, the exogenous effector can silence expression of a factor such as an interferon. The examples further describe how curons can be made by inserting exogenous effectors into sequences derived, e.g., from Anellovirus. It is on the basis of these examples that the description hereinafter contemplates various variations of the specific findings and combinations considered in the examples. For example, the skilled person will understand from the examples that the specific miRNAs are used just as an example of an exogenous effector and that other exogenous effectors may be, e.g., other regulatory nucleic acids or therapeutic peptides. Similarly, the specific capsids used in the examples may be replaced by substantially non-pathogenic proteins described hereinafter. The specific Anellovirus sequences described in the examples may also be replaced by the Anellovirus sequences described hereinafter. These considerations similarly apply to protein binding sequences, regulatory sequences such as promoters, and the like. Independent thereof, the person skilled in the art will in particular consider such embodiments which are closely related to the examples.

In some embodiments, a curon, or the genetic element comprised in the curon, is introduced into a cell (e.g., a human cell). In some embodiments, the exogenous effector (e.g., an RNA, e.g., an miRNA), e.g., encoded by the genetic element of a curon, is expressed in a cell (e.g., a human cell), e.g., once the curon or the genetic element has been introduced into the cell, e.g., as described in Example 19. In embodiments, introduction of the curon, or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell (e.g., as described in Example 22). In embodiments, introduction of the curon, or genetic element comprised therein, decreases level of interferon produced by the cell, e.g., as described in Examples 3 and 4. In embodiments, introduction of the curon, or genetic element comprised therein, into a cell modulates (e.g., increases or decreases) a function of the cell. In embodiments, introduction of the curon, or genetic element comprised therein, into a cell modulates (e.g., increases or decreases) the viability of the cell. In embodiments, introduction of the curon, or genetic element comprised therein, into a cell decreases viability of a cell (e.g., a cancer cell), e.g., as described in Example 22.

In some embodiments, a curon (e.g., a synthetic curon) described herein induces an antibody prevalence of less than 70% (e.g., less than about 60%, 50%, 40%, 30%, 20%, or 10% antibody prevalence). In embodiments, antibody prevalence is determined according to methods known in the art. In embodiments, antibody prevalence is determined by detecting antibodies against an Anellovirus (e.g., as described herein), or a curon based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anellovirus or a curon based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

### Anelloviruses

In some embodiments, a synthetic curon, e.g., as described herein, comprises sequences or expression products derived from an Anellovirus. Generally, a synthetic curon includes one or more sequences or expression products that are exogenous relative to the Anellovirus. The Anellovirus genus was once classified as a clade within the Circoviridae family, and has more recently been classified as a separate family. Anelloviruses generally have single-stranded circular DNA genomes with negative polarity. Anellovirus has not been linked to any human disease. However, attempts to link Anellovirus infection with human disease are confounded by the high incidence of asymptomatic Anellovirus viremia in control cohort population(s), the remarkable genomic diversity within the anellovirus viral family, the historical inability to propagate the agent in vitro, and the lack of animal model(s) of Anellovirus disease (Yzebe et al., Panminerva Med. (2002) 44:167-177; Biagini, P., Vet. Microbiol. (2004) 98:95-101).

Anellovirus appears to be transmitted by oronasal or fecal-oral infection, mother-to-infant and/or in utero transmission (Gerner et al., Ped. Infect. Dis. J. (2000) 19:1074-1077). Infected persons are characterized by a prolonged (months to years) Anellovirus viremia. Humans may be co-infected with more than one genogroup or strain (Saback, et al., Scad. J. Infect. Dis. (2001) 33:121-125). There is a suggestion that these genogroups can recombine within infected humans (Rey et al., Infect. (2003) 31:226-233). The double stranded isoform (replicative) intermediates have been found in several tissues, such as liver, peripheral blood mononuclear cells and bone marrow (Kikuchi et al., J. Med. Virol. (2000) 61:165-170; Okamoto et al., Biochem. Biophys. Res. Commun. (2002) 270:657-662; Rodriguez-lnigo et al., Am. J. Pathol. (2000) 156:1227-1234).

In some embodiments, a curon as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the Anellovirus sequence is selected from a sequence as shown in any of Tables 1, 3, 5, 7, 9, 11, or 13. In some embodiments, a curon as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to a TATA box, cap site, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of any of the Anelloviruses described herein (e.g., an Anellovirus sequence as annotated, or as encoded by a sequence listed, in any of Tables 1-16 or 19). In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein (e.g., an Anellovirus sequence as annotated, or as encoded by a sequence listed, in any of Tables 1-16 or 19). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 or ORF2 protein (e.g., an ORF1 or ORF2 amino acid sequence as shown in any of Tables 2, 4, 6, 8, 10, 12, 14, or 16, or an ORF1 or ORF2 amino acid sequence encoded by a nucleic acid sequence as shown in any of Tables 1, 3, 5, 7, 9, 11, 13, 15, or 19).

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of Table 1 (e.g., nucleotides 571-2613 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table 1 (e.g., nucleotides 571-587 and/or 2137-2613 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table 1 (e.g., nucleotides 571-687 and/or 2339-2659 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table 1 (e.g., nucleotides 299-691 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table 1 (e.g., nucleotides 299-687 and/or 2137-2659 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table 1 (e.g., nucleotides 299-687 and/or 2339-2831 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2t/3 nucleotide sequence of Table 1 (e.g., nucleotides 299-348 and/or 2339-2831 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table 1 (e.g., nucleotides 84-90 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus Cap site nucleotide sequence of Table 1 (e.g., nucleotides 107-114 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table 1 (e.g., nucleotide 114 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table 1 (e.g., nucleotides 177-247 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table 1 (e.g., nucleotides 2325-2610 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table 1 (e.g., nucleotides 2813-2818 of the nucleic acid sequence of Table 1). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table 1 (e.g., nucleotides 3415-3570 of the nucleic acid sequence of Table 1).

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of Table 3 (e.g., nucleotides 599-2839 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table 3 (e.g., nucleotides 599-727 and/or 2381-2839 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table 3 (e.g., nucleotides 599-727 and/or 2619-2813 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table 3 (e.g., nucleotides 357-731 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table 3 (e.g., nucleotides 357-727 and/or 2381-2813 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table 3 (e.g., nucleotides 357-727 and/or 2619-3021 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2t/3 nucleotide sequence of Table 3 (e.g., nucleotides 357-406 and/or 2619-3021 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table 3 (e.g., nucleotides 89-90 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus Cap site nucleotide sequence of Table 3 (e.g., nucleotides 107-114 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table 3 (e.g., nucleotide 114 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table 3 (e.g., nucleotides 174-244 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table 3 (e.g., nucleotides 2596-2810 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table 3 (e.g., nucleotides 3017-3022 of the nucleic acid sequence of Table 3). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table 3 (e.g., nucleotides 3691-3794 of the nucleic acid sequence of Table 3).

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of Table 5 (e.g., nucleotides 599-2830 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table 5 (e.g., nucleotides 599-715 and/or 2363-2830 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table 5 (e.g., nucleotides 599-715 and/or 2565-2789 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table 5 (e.g., nucleotides 336-719 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table 5 (e.g., nucleotides 336-715 and/or 2363-2789 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table 5 (e.g., nucleotides 336-715 and/or 2565-3015 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2t/3 nucleotide sequence of Table 5 (e.g., nucleotides 336-388 and/or 2565-3015 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table 5 (e.g., nucleotides 83-88 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus Cap site nucleotide sequence of Table 5 (e.g., nucleotides 104-111 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table 5 (e.g., nucleotide 111 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table 5 (e.g., nucleotides 170-240 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table 5 (e.g., nucleotides 2551-2786 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table 5 (e.g., nucleotides 3011-3016 of the nucleic acid sequence of Table 5). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table 5 (e.g., nucleotides 3632-3753 of the nucleic acid sequence of Table 5).

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of Table 7 (e.g., nucleotides 590-2899 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table 7 (e.g., nucleotides 590-712 and/or 2372-2899 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table 7 (e.g., nucleotides 590-712 and/or 2565-2873 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table 7 (e.g., nucleotides 354-716 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table 7 (e.g., nucleotides 354-712 and/or 2372-2873 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table 7 (e.g., nucleotides 354-712 and/or 2565-3075 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2t/3 nucleotide sequence of Table 7 (e.g., nucleotides 354-400 and/or 2565-3075 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table 7 (e.g., nucleotides 86-90 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus Cap site nucleotide sequence of Table 7 (e.g., nucleotides 107-114 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table 7 (e.g., nucleotide 114 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table 7 (e.g., nucleotides 174-244 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table 7 (e.g., nucleotides 2551-2870 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table 7 (e.g., nucleotides 3071-3076 of the nucleic acid sequence of Table 7). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table 7 (e.g., nucleotides 3733-3853 of the nucleic acid sequence of Table 7).

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of Table 9 (e.g., nucleotides 577-2787 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table 9 (e.g., nucleotides 577-699 and/or 2311-2787 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table 9 (e.g., nucleotides 577-699 and/or 2504-2806 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table 9 (e.g., nucleotides 341-703 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table 9 (e.g., nucleotides 341-699 and/or 2311-2806 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table 9 (e.g., nucleotides 341-699 and/or 2504-2978 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2t/3 nucleotide sequence of Table 9 (e.g., nucleotides 341-387 and/or 2504-2978 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table 9 (e.g., nucleotides 83-87 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus Cap site nucleotide sequence of Table 9 (e.g., nucleotides 104-111 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table 9 (e.g., nucleotide 111 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table 9 (e.g., nucleotides 171-241 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table 9 (e.g., nucleotides 2463-2784 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table 9 (e.g., nucleotides 2974-2979 of the nucleic acid sequence of Table 9). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table 9 (e.g., nucleotides 3644-3758 of the nucleic acid sequence of Table 9).

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of Table 11 (e.g., nucleotides 612-2612 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table 11 (e.g., nucleotides 612-719 and/or 2274-2612 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table 11 (e.g., nucleotides 612-719 and/or 2449-2589 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table 11 (e.g., nucleotides 424-723 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table 11 (e.g., nucleotides 424-719 and/or 2274-2589 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table 11 (e.g., nucleotides 424-719 and/or 2449-2812 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table 11 (e.g., nucleotides 237-243 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus Cap site nucleotide sequence of Table 11 (e.g., nucleotides 260-267 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table 11 (e.g., nucleotide 267 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table 11 (e.g., nucleotides 323-393 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table 11 (e.g., nucleotides 2441-2586 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table 11 (e.g., nucleotides 2808-2813 of the nucleic acid sequence of Table 11). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table 11 (e.g., nucleotides 2868-2929 of the nucleic acid sequence of Table 11).

In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of Table 13 (e.g., nucleotides 432-2453 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/1 nucleotide sequence of Table 13 (e.g., nucleotides 432-584 and/or 1977-2453 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1/2 nucleotide sequence of Table 13 (e.g., nucleotides 432-584 and/or 2197-2388 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of Table 13 (e.g., nucleotides 283-588 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/2 nucleotide sequence of Table 13 (e.g., nucleotides 283-584 and/or 1977-2388 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2/3 nucleotide sequence of Table 13 (e.g., nucleotides 283-584 and/or 2197-2614 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus TATA box nucleotide sequence of Table 13 (e.g., nucleotides 21-25 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus Cap site nucleotide sequence of Table 13 (e.g., nucleotides 42-49 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus transcriptional start site nucleotide sequence of Table 13 (e.g., nucleotide 49 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of Table 13 (e.g., nucleotides 117-187 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus three open-reading frame region nucleotide sequence of Table 13 (e.g., nucleotides 2186-2385 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus poly(A) signal nucleotide sequence of Table 13 (e.g., nucleotides 2676-2681 of the nucleic acid sequence of Table 13). In embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich nucleotide sequence of Table 13 (e.g., nucleotides 3054-3172 of the nucleic acid sequence of Table 13).

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of Table 2. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/1 amino acid sequence of Table 2. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/2 amino acid sequence of Table 2. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of Table 2. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/2 amino acid sequence of Table 2. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/3 amino acid sequence of Table 2. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2t/3 amino acid sequence of Table 2.

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of Table 4. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/1 amino acid sequence of Table 4. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/2 amino acid sequence of Table 4. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of Table 4. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/2 amino acid sequence of Table 4. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/3 amino acid sequence of Table 4. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2t/3 amino acid sequence of Table 4.

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of Table 6. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/1 amino acid sequence of Table 6. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/2 amino acid sequence of Table 6. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of Table 6. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/2 amino acid sequence of Table 6. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/3 amino acid sequence of Table 6. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2t/3 amino acid sequence of Table 6.

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of Table 8. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/1 amino acid sequence of Table 8. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/2 amino acid sequence of Table 8. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of Table 8. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/2 amino acid sequence of Table 8. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/3 amino acid sequence of Table 8. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2t/3 amino acid sequence of Table 8.

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of Table 10. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/1 amino acid sequence of Table 10. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/2 amino acid sequence of Table 10. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of Table 10. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/2 amino acid sequence of Table 10. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/3 amino acid sequence of Table 10. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2t/3 amino acid sequence of Table 10.

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of Table 12. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/1 amino acid sequence of Table 12. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/2 amino acid sequence of Table 12. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of Table 12. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/2 amino acid sequence of Table 12. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/3 amino acid sequence of Table 12.

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of Table 14. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/1 amino acid sequence of Table 14. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1/2 amino acid sequence of Table 14. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of Table 14. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/2 amino acid sequence of Table 14. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2/3 amino acid sequence of Table 14.

In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of Table 2. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/1 amino acid sequence of Table 2. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/2 amino acid sequence of Table 2. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of Table 2. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/2 amino acid sequence of Table 2. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/3 amino acid sequence of Table 2. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2t/3 amino acid sequence of Table 2.

In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of Table 4. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/1 amino acid sequence of Table 4. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/2 amino acid sequence of Table 4. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of Table 4. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/2 amino acid sequence of Table 4. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/3 amino acid sequence of Table 4. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2t/3 amino acid sequence of Table 4.

In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of Table 6. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/1 amino acid sequence of Table 6. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/2 amino acid sequence of Table 6. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of Table 6. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/2 amino acid sequence of Table 6. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/3 amino acid sequence of Table 6. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2t/3 amino acid sequence of Table 6.

In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of Table 8. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/1 amino acid sequence of Table 8. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/2 amino acid sequence of Table 8. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of Table 8. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/2 amino acid sequence of Table 8. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/3 amino acid sequence of Table 8. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2t/3 amino acid sequence of Table 8.

In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of Table 10. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/1 amino acid sequence of Table 10. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/2 amino acid sequence of Table 10. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of Table 10. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/2 amino acid sequence of Table 10. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/3 amino acid sequence of Table 10. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2t/3 amino acid sequence of Table 10.

In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of Table 12. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/1 amino acid sequence of Table 12. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/2 amino acid sequence of Table 12. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of Table 12. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/2 amino acid sequence of Table 12. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/3 amino acid sequence of Table 12.

In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of Table 14. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/1 amino acid sequence of Table 14. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF1/2 amino acid sequence of Table 14. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of Table 14. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/2 amino acid sequence of Table 14. In embodiments, the curon described herein comprises a protein having an amino acid sequence having at least about 70%, 75%, 80%, 85%, 90%, 95%, 96%, 97%, 98%, 99%, or 100% sequence identity to the Anellovirus ORF2/3 amino acid sequence of Table 14.

TABLE 1
Exemplary Anellovirus nucleic acid sequence
(Alphatorquevirus, Clade 1)

| | |
|---|---|
| Name | TTV-CT30F |
| Genus/Clade | Alphatorquevirus , Clade 1 |
| Accession Number | AB064597.1 |
| Full Sequence: | 3570 bp |

1 10 20 30 40 50
| | | | | |
ATTTTGTGCAGCCCGCCAATTCTCGTTCAAACAGGCCAATCAGGAGGCTC
TACGTACACTTCCTGGGGTGTGTCTTCGAAGAGTATATAAGCAGAGGCGG
TGACGAATGGTAGAGTTTTTCCTGGCCCGTCCGCGGCGAGAGCGCGAGCG
GAGCGAGCGATCGAGCGTCCCGTGGGCGGGTGCCGTAGGTGAGTTTACAC
ACCGCAGTCAAGGGGCAATTCGGGCTCGGGACTGGCCGGGCTATGGGCAA
GATTCTTAAAAAATTCCCCCGATCCCTCTGTCGCCAGGACATAAAAACAT
GCCGTGGAGACCGCCGGTGCATAGTGTCCAGGGGCGAGAGGATCAGTGGT
TCGCGAGCTTTTTTCACGGCCACGCTTCATTTTGCGGTTGCGGTGACGCT
GTTGGCCATCTTAATAGCATTGCTCCTCGCTTTCCTCGCGCCGGTCCACC
AAGGCCCCCTCCGGGGCTAGAGCAGCCTAACCCCCCGCAGCAGGGCCCGG
CCGGGCCCGGAGGGCCGCCCGCCATCTTGGCGCTGCCGGCTCCGCCCGCG
GAGCCTGACGACCCGCAGCCACGGCGTGGTGGTGGGGACGGTGGCGCCGC
CGCTGGCGCCGCAGGCGACCGTGGAGACCGAGACTACGACGAAGAAGAGC
TAGACGAGCTTTTCCGCGCCGCCGCCGAAGACGATTTGTAAGTAGGAGAT
GGCGCCGGCCTTACAGGCGCAGGAGGAGACGCGGGCGACGCAGACGCAGA
CGCAGACGCAGACATAAGCCCACCCTAGTACTCAGACAGTGGCAACCTGA
CGTTATCAGACACTGTAAGATAACAGGACGGATGCCCCTCATTATCTGTG
GAAAGGGGTCCACCCAGTTCAACTACATCACCCACGCGGACGACATCACC
CCCAGGGGAGCCTCCTACGGGGGCAACTTCACAAACATGACTTTCTCCCT
GGAGGCAATATACGAACAGTTTCTGTACCACAGAAACAGGTGGTCAGCCT
CCAACCACGACCTCGAACTCTGCAGATACAAGGGTACCACCCTAAAACTG
TACAGGCACCCAGATGTAGACTACATAGTCACCTACAGCAGAACGGGACC
CTTTGAGATCAGCCACATGACCTACCTCAGCACTCACCCCCTTCTCATGC
TGCTAAACAAACACCACATAGTGGTGCCCAGCCTAAAGACTAAGCCCAGG
GGCAGAAAGGCCATAAAAGTCAGAATAAGACCCCCCAAACTCATGAACAA
CAAGTGGTACTTCACCAGAGACTTCTGTAACATAGGCCTCTTCCAGCTCT
GGGCCACAGGCTTAGAACTCAGAAACCCCTGGCTCAGAATGAGCACCCTG
AGCCCCTGCATAGGCTTCAATGTCCTTAAAAACAGCATTTACACAAACCT
CAGCAACCTACCTCAGCACAGAGAAGACAGACTTAACATTATTAACAACA
CATTACACCCACATGACATAACAGGACCAAACAATAAAAAATGGCAGTAC
ACATATACCAAACTCATGGCCCCCATTTACTATTCAGCAAACAGGGCCAG
CACCTATGACTTACTACGAGAGTATGGCCTCTACAGTCCATACTACCTAA
ACCCCACAAGGATAAACCTTGACTGGATGACCCCCTACACACACGTCAGG
TACAATCCACTAGTAGACAAGGGCTTCGGAAACAGAATATACATACAGTG
GTGCTCAGAGGCAGATGTAAGCTACAACAGGACTAAATCCAAGTGTCTCT
TACAAGACATGCCCCTGTTTTTCATGTGCTATGGCTACATAGACTGGGCA
ATTAAAAACACAGGGGTCTCCTCACTAGCGAGAGACGCCAGAATCTGCAT
CAGGTGTCCCTACACAGAGCCACAGCTGGTGGGCTCCACAGAAGACATAG
GGTTCGTACCCATCACAGAGACCTTCATGAGGGGCGACATGCCGGTACTT
GCACCATACATACCGTTGAGCTGGTTTTGCAAGTGGTATCCCAACATAGC
TCACCAGAAGGAAGTACTTGAGGCAATCATTTCCTGCAGCCCCTTCATGC
CCCGTGACCAGGGCATGAACGGTTGGGATATTACAATAGGTTACAAAATG
GACTTCTTATGGGGCGGTTCCCCTCTCCCCTCACAGCCAATCGACGACCC
CTGCCAGCAGGGAACCCACCCGATTCCCGACCCCGATAAGCACCCTCGCC
TCCTACAAGTGTCGAACCCGAAACTGCTCGGACCGAGGACAGTGTTCCAC
AAGTGGGACATCAGACGTGGGCAGTTTAGCAAAAGAAGTATTAAAAGAGT
GTCAGAATACTCATCGGATGATGAATCTCTTGCGCCAGGTCTCCCATCAA
AGCGAAACAAGCTCGACTCGGCCTTCAGAGGAGAAAACCCAGAGCAAAAA
GAATGCTATTCTCTCCTCAAAGCACTCGAGGAAGAAGAGACCCCAGAAGA
AGAAGAACCAGCACCCCAAGAAAAAGCCCAGAAAGAGGAGCTACTCCACC
AGCTCCAGCTCCAGAGACGCCACCAGCGAGTCCTCAGACGAGGGCTCAAG
CTCGTCTTTACAGACATCCTCCGACTCCGCCAGGGAGTCCACTGGAACCC
CGAGCTCACATAGAGCCCCCACCTTACATACCAGACCTACTTTTTCCCAA
TACTGGTAAAAAAAAAAAATTCTCTCCCTTCGACTGGGAAACGGAGGCCC
AGCTAGCAGGGATATTCAAGCGTCCTATGCGCTTCTATCCCTCAGACACC
CCTCACTACCCGTGGTTACCCCCCAAGCGCGATATCCCGAAAATATGTAA
CATAAACTTCAAAATAAAGCTGCAAGAGTGAGTGATTCGAGGCCCTCCTC
TGTTCACTTAGCGGTGTCTACCTCTTAAAGTCACCAAGCACTCCGAGCGT
CAGCGAGGAGTGCGACCCTCCACCAAGGGGCAACTTCCTCGGGGTCCGGC
GCTACGCGCTTCGCGCTGCGCCGGACGCCTCGGACCCCCCCCCGACCCGA
ATCGCTCGCGCGATTCGGACCTGCGGCCTCGGGGGGGGTCGGGGGCTTTA
CTAAACAGACTCCGAGTTGCCACTGGACTCAGGAGCTGTGAATCAGTAAC
GAAAGTGAGTGGGGCCAGACTTCGCCATAGGGCCTTTAACTTGGGGTCGT
CTGTCGGTGGCTTCCGGGTCCGCCTGGGCGCCGCCATTTTAGCTTTAGAC
GCCATTTTAGGCCCTCGCGGGCACCCGTAGGCGCGTTTTAATGACGTCAC
GGCAGCCATTTTGTCGTGACGTTTGAGACACGTGATGGGGGCGTGCCTAA
ACCCGGAAGCATCCCTGGTCACGTGACTCTGACGTCACGGCGGCCATTTT
GTGCTGTCCGCCATCTTGTGACTTCCTTCCGCTTTTTCAAAAAAAAAGAG
GAAGTATGACAGTAGCGGCGGGGGGGCGGCCGCGTTCGCGCGCCGCCCAC
CAGGGGGTGCTGCGCGCCCCCCCCCGCGCATGCGCGGGGCCCCCCCCCGG
GGGGGCTCCGCCCCCCCGGCCCCCCCCCGTGCTAAACCCACCGCGCATGC
GCGACCACGCCCCCGCCGCC (SEQ ID NO: 1)

| | |
|---|---|
| Annotations: | |
| Putative Domain | Base range |
| TATA Box | 84-90 |
| Cap Site | 107-114 |
| Transcriptional Start Site | 114 |
| 5′ UTR Conserved Domain | 177-247 |
| ORF2 | 299-691 |
| ORF2/2 | 299-687; 2137-2659 |
| ORF2/3 | 299-687; 2339-2831 |
| ORF2t/3 | 299-348; 2339-2831 |
| ORF1 | 571-2613 |
| ORF1/1 | 571-687; 2137-2613 |
| ORF1/2 | 571-687; 2339-2659 |
| Three open-reading frame region | 2325-2610 |
| Poly(A) Signal | 2813-2818 |
| GC-rich region | 3415-3570 |

TABLE 2
Exemplary Anellovirus amino acid sequences (Alphatorquevirus, Clade 1)
TTV-CT30F (Alphatorquevirus Clade 1)

| | |
|---|---|
| ORF2 | MPWRPPVHSVQGREDQWFASFFHGHASFCGCGDAVGHLNSIAPRFPRAGPPRPPPG |
| | LEQPNPPQQGPAGPGGPPAILALPAPPAEPDDPQPRRGGGDGGAAAGAAGDRGDRD |
| | YDEEELDELFRAAAEDDL (SEQ ID NO: 2) |
| ORF2/2 | MPWRPPVHSVQGREDQWFASFFHGHASFCGCGDAVGHLNSIAPRFPRAGPPRPPPG |
| | LEQPNPPQQGPAGPGGPPAILALPAPPAEPDDPQPRRGGGDGGAAAGAAGDRGDRD |
| | YDEEELDELFRAAAEDDFQSTTPASREPTRFPTPISTLASYKCRTRNCSDRGQCSTSG |
| | TSDVGSLAKEVLKECQNTHRMMNLLRQVSHQSETSSTRPSEEKTQSKKNAILSSKH |
| | SRKKRPQKKKNQHPKKKPRKRSYSTSSSSRDATSESSDEGSSSSLQTSSDSARESTGT |
| | PSSHRAPTLHTRPTFSQYW (SEQ ID NO: 3) |
| ORF2/3 | MPWRPPVHSVQGREDQWFASFFHGHASFCGCGDAVGHLNSIAPRFPRAGPPRPPPG |
| | LEQPNPPQQGPAGPGGPPAILALPAPPAEPDDPQPRRGGGDGGAAAGAAGDRGDRD |
| | YDEEELDELFRAAAEDDLSPIKAKQARLGLQRRKPRAKRMLFSPQSTRGRRDPRRR |
| | RTSTPRKSPERGATPPAPAPETPPASPQTRAQARLYRHPPTPPGSPLEPRAHIEPPPYIP |
| | DLLFPNTGKKKKFSPFDWETEAQLAGIFKRPMRFYPSDTPHYPWLPPKRDIPKICNIN |
| | FKIKLQE (SEQ ID NO: 4) |
| ORF2t/3 | MPWRPPVHSVQGREDQWSPIKAKQARLGLQRRKPRAKRMLFSPQSTRGRRDPRRR |
| | RTSTPRKSPERGATPPAPAPETPPASPQTRAQARLYRHPPTPPGSPLEPRAHIEPPPYIP |
| | DLLFPNTGKKKKFSPFDWETEAQLAGIFKRPMRFYPSDTPHYPWLPPKRDIPKICNIN |
| | FKIKLQE (SEQ ID NO: 5) |
| ORF1 | TAWWWGRWRRRWRRRRPWRPRLRRRRARRAFPRRRRRRFVSRRWRRPYRRRRR |
| | RGRRRRRRRRRHKPTLVLRQWQPDVIRHCKITGRMPLIICGKGSTQFNYITHADDIT |
| | PRGASYGGNFTNMTFSLEAIYEQFLYHRNRWSASNHDLELCRYKGTTLKLYRHPD |
| | VDYIVTYSRTGPFEISHMTYLSTHPLLMLLNKHHIVVPSLKTKPRGRKAIKVRIRPPK |
| | LMNNKWYFTRDFCNIGLFQLWATGLELRNPWLRMSTLSPCIGFNVLKNSIYTNLSN |
| | LPQHREDRLNIINNTLHPHDITGPNNKKWQYTYTKLMAPIYYSANRASTYDLLREY |
| | GLYSPYYLNPTRINLDWMTPYTHVRYNPLVDKGFGNRIYIQWCSEADVSYNRTKSK |
| | CLLQDMPLFFMCYGYIDWAIKNTGVSSLARDARICIRCPYTEPQLVGSTEDIGFVPIT |
| | ETFMRGDMPVLAPYIPLSWFCKWYPNIAHQKEVLEAIISCSPFMPRDQGMNGWDITI |
| | GYKMDFLWGGSPLPSQPIDDPCQQGTHPIPDPDKHPRLLQVSNPKLLGPRTVFHKW |
| | DIRRGQFSKRSIKRVSEYSSDDESLAPGLPSKRNKLDSAFRGENPEQKECYSLLKALE |
| | EEETPEEEEPAPQEKAQKEELLHQLQLQRRHQRVLRRGLKLVFTDILRLRQGVHWN |
| | PELT (SEQ ID NO: 6) |
| ORF 1/1 | TAWWWGRWRRRWRRRRPWRPRLRRRRARRAFPRRRRRRFPIDDPCQQGTHPIPDP |
| | DKHPRLLQVSNPKLLGPRTVFHKWDIRRGQFSKRSIKRVSEYSSDDESLAPGLPSKR |
| | NKLDSAFRGENPEQKECYSLLKALEEEETPEEEEPAPQEKAQKEELLHQLQLQRRH |
| | QRVLRRGLKLVFTDILRLRQGVHWNPELT (SEQ ID NO: 7) |
| ORF 1/2 | TAWWWGRWRRRWRRRRPWRPRLRRRRARRAFPRRRRRRFVSHQSETSSTRPSEE |
| | KTQSKKNAILSSKHSRKKRPQKKKNQHPKKKPRKRSYSTSSSSRDATSESSDEGSSS |
| | SLQTSSDSARESTGTPSSHRAPTLHTRPTFSQYW (SEQ ID NO: 8) |

TABLE 3
Exemplary Anellovirus nucleic acid sequence
(Alphatorquevirus, Clade 2)

| | |
|---|---|
| Name | TTV-TJN02 |
| Genus/Clade | Alphatorquevirus , Clade 2 |
| Accession Number | AB028669.1 |
| Full Sequence: | 3794 bp |

1 10 20 30 40 50
| | | | | |
CCCGAAGTCCGTCACTAACCACGTGACTCCTGTCGCCCAATCAGAGTGTA
TGTCGTGCATTTCCTGGGCATGGTCTACATCCTGATATAACTAAGTGCAC
TTCCGAATGGCTGAGTTTTCCACGCCCGTCCGCAGCGAGGGAGCGACGGA
GGAGCTCCCGAGCGTCCCGAGGGCGGGTGCCGGAGGTGAGTTTACACACC
GCAGTCAAGGGGCAATTCGGGCTCGGGACTGGCCGGGCTATGGGCAAGGC
TCTTAGGGTCTTCATTCTTAATATGTTTCTTGGCAGAGTTTACCGCCACA
AGAAAAGGAAAGTGCTACTGTCCACACTGCGAGCTCCACAGGCGTCTCGC
AGGGCTATGAGTTGGCGACCCCCGGTACACGATGCACCCGGCATCGAGCG
CAATTGGTACGAGGCCTGTTTCAGAGCCCACGCTGGAGCTTGTGGCTGTG
GCAATTTTATTATGCACCTTAATCTTTTGGCTGGGCGTTATGGTTTTACT
CCGGGGTCAGCGCCGCCAGGTGGTCCTCCTCCGGGCACCCCGCAGATAAG
GAGAGCCAGGCCTAGTCCCGCCGCACCAGAGCAGCCCGCTGCCCTACCAT
GGCATGGGGATGGTGGAGATGGCGGCGCCGCTGGCCCGCCAGACGCTGGA
GGAGACGCCGTCGCCGGCGCCCCGTACGGAGAACAAGAGCTCGCCGACCT
GCTCGACGCTATAGAAGACGACGAACAGTAAGAACCAGGCGAAGGCGGTG
GGGGCGCAGACGGTACAGACGGGGCTGGAGACGCAGGACTTATGTGAGAA
AGGGGCGACACAGAAAAAAGAAAAAGAGACTGATACTGAGACAGTGGCAA
CCAGCCACAAGACGCAGATGTACCATAACTGGGTACCTGCCCATAGTGTT
CTGCGGCCACACTAGGGGCAATAAAAACTATGCACTACACTCTGACGACT
ACACCCCCCAAGGACAACCATTTGGAGGGGCTCTAAGCACTACCTCATTC
TCTTTAAAAGTACTATTTGACCAGCATCAGAGAGGACTAAACAAGTGGTC
TTTTCCAAACGACCAACTAGACCTCGCCAGATATAGAGGCTGCAAATTTA
TATTTTATAGAACAAAACAAACTGACTGGGTGGGCCAGTATGACATATCA
GAACCCTACAAGCTAGACAAATACAGCTGCCCCAACTATCACCCTGGAAA
CATGATTAAGGCAAAGCACAAATTTTTAATACCAAGCTATGACACTAATC
CTAGAGGCAGACAAAAAATTATAGTTAAAATTCCCCCCCCAGACCTCTTT
GTAGACAAGTGGTACACTCAAGAGGATCTGTGTTCCGTTAATCTTGTGTC
ACTTGCGGTTTCTGCGGCTTCCTTTCTCCACCCATTCGGCTCACCACAAA
CTGACAACCCTTGCTACACCTTCCAGGTGTTGAAAGAGTTCTACTATCAG
GCAATAGGCTTCTCTGCAAGCACACAAGCAATGACATCAGTATTAGACAC
GCTATACACACAAAACAGTTATTGGGAATCTAATCTAACTCAGTTTTATG
TACTTAATGCAAAAAAAGGCAGTGATACAACACAGCCTTTAACTAGCAAT
ATGCCAACTCGTGAAGAGTTTATGGCAAAAAAAAATACCAATTACAACTG
GTATACATACAAGGCCGCGTCAGTAAAAAATAAACTACATCAAATGAGAC
AAACCTATTTTGAGGAGTTAACCTCTAAGGGGCCACAAACAACAAAAAGT
GAGGAAGGCTACAGTCAGCACTGGACCACCCCCTCCACAAACGCCTACGA
ATATCACTTAGGAATGTTTAGTGCAATATTTCTAGCCCCAGACAGGCCAG
TACCTAGATTTCCATGCGCCTACCAAGATGTAACTTACAACCCCTTAATG
GACAAAGGGGTGGGAAACCACATTTGGTTTCAGTACAACACAAAGGCAGA
CACTCAGCTAATAGTCACAGGAGGGTCCTGCAAAGCACACATACAAGACA
TACCACTGTGGGCGGCCTTCTATGGATACAGTGACTTTATAGAGTCAGAA
CTAGGCCCCTTTGTAGATGCAGAGACGGTAGGCTTAGTGTGTGTAATATG
CCCTTATACAAAACCCCCCATGTACAACAAGACAAACCCCGCCATGGGCT
ACGTGTTCTATGACAGAAACTTTGGTGACGGAAAATGGACTGACGGACGG
GGCAAAATAGAGCCCTACTGGCAAGTTAGGTGGAGGCCCGAAATGCTTTT
CCAAGAAACTGTAATGGCAGACCTAGTTCAGACTGGGCCCTTTAGCTACA
AAGACGAACTTAAAAACAGCACCCTAGTGTGCAAGTACAAATTCTATTTC
ACCTGGGGAGGTAACATGATGTTCCAACAGACGATCAAAAACCCGTGCAA
GACGGACGGACAACCCACCGACTCCAGTAGACACCCTAGAGGAATACAAG
TGGCGGACCCGGAACAAATGGGACCCCGCTGGGTGTTCCACTCCTTTGAC
TGGCGAAGGGGCTATCTTAGCGAGAAAGCTCTCAAACGCCTGCAAGAAAA
ACCTCTTGACTATGACGAATATTTTACACAACCAAAAAGACCTAGAATCT
TTCCTCCAACAGAATCAGCAGAGGGAGAGTTCCGAGAGCCCGAAAAAGGC
TCGTATTCAGAGGAAGAAAGGTCGCAAGCCTCTGCCGAAGAGCAGACGCA
GGAGGCGACAGTACTCCTCCTCAAGCGACGACTCAGAGAGCAACAGCAGC
TCCAGCAGCAGCTCCAATTCCTCACCCGAGAAATGTTCAAAACGCAAGCG
GGTCTCCACCTAAACCCTATGTTATTAAACCAGCGATAAACCAAGTGTAC
CTGTTTCCAGAGAGGGCCCCAAAACCCCCTCCTAGCAGCCAAGACTGGCA
GCAGGAGTACGAGGCCTGCGCAGCCTGGGACAGGCCCCCTAGATACAATC
TGTCCTCTCCTCCTTTCTACCCCAGCTGCCCTTCAAAATTCTGTGTAAAA
TTCAGCCTTGGCTTTAAATAAATGGCAACTTTACTGTGCAAGGCCGTGGG
AGTTTCACTGGTCGGTGTCTACCTCTAAAGGTCACTAAGCACTCCGAGCG
TTAGCGAGGAGTGCGACCCTTCCCCCTGACTCAACTTCTTCGGAGCCGCG
CGCTACGCCTTCGGCTGCGCGCGGCACCTCAGACCCCCGCTCGTGCTGAC
ACGCTCGCGCGTGTCAGACCACTTCGGGCTCGCGGGGGTCGGGAATTTTG
CTAAACAGACTCCGAGTTGCTCTTGGACACTGAGGGGGCATATCAGTAAC
GAAAGTGAGTGGGGCCAGACTTCGCCATAAGGCCTTTATCTTCTTGCCAT
TGGATAGTATCGAGGGTTGCCATAGGCTTCGACCTCCATTTTAGGCCTTC
CGGACTACAAAAATGGCCGTTTTAGTGACGTCACGGCCGCCATTTTAAGT
AAGGCGGAAGCAGCTCGGCGTACACAAAATGGCGGCGGAGCACTTCCGGC
TTGCCCAAAATGGTGGGCAACTTCTTCCGGGTCAAAGGTCACAGCTACGT
CACAAGTCACGTGGGGAGGGTTGGCGTTTAACCCGGAAGCCAATCCTCTT
ACGTGGCCTGTCACGTGACTTGTACGTCACGACCACCATTTTGTTTTACA
AAATGGCCGACTTCCTTCCTCTTTTTTAAAAATAACGGTTCGGCGGCGGC
GCGCGCGCTACGCGCGCGCGCCGGGGGGCTGCCGCCCCCCCCCCGCGCAT
GCGCGGGGCCCCCCCCCGCGGGGGGCTCCGCCCCCCGGCCCCCC
(SEQ ID NO: 9)

| | |
|---|---|
| Annotations: | |
| Putative Domain | Base range |
| TATA Box | 89-90 |
| Cap Site | 107-114 |
| Transcriptional Start Site | 114 |
| 5′ UTR Conserved Domain | 174-244 |
| ORF2 | 357-731 |
| ORF2/2 | 357-727; 2381-2813 |
| ORF2/3 | 357-727; 2619-3021 |
| ORF2t/3 | 357-406; 2619-3021 |
| ORF1 | 599-2839 |
| ORF1/1 | 599-727; 2381-2839 |
| ORF1/2 | 599-727; 2619-2813 |
| Three open-reading frame region | 2596-2810 |
| Poly(A) Signal | 3017-3022 |
| GC-rich region | 3691-3794 |

TABLE 4
Exemplary Anellovirus amino acid sequences (Alphatorquevirus, Clade 2)
TTV-TJN02 (Alphatorquevirus Clade 2)

| | |
|---|---|
| ORF2 | MSWRPPVHDAPGIERNWYEACFRAHAGACGCGNFIMHLNLLAGRYGFTPGSAPPG |
| | GPPPGTPQIRRARPSPAAPEQPAALPWHGDGGDGGAAGPPDAGGDAVAGAPYGEQ |
| | ELADLLDAIEDDEQ (SEQ ID NO: 10) |
| ORF2/2 | MSWRPPVHDAPGIERNWYEACFRAHAGACGCGNFIMHLNLLAGRYGFTPGSAPPG |
| | GPPPGTPQIRRARPSPAAPEQPAALPWHGDGGDGGAAGPPDAGGDAVAGAPYGEQ |
| | ELADLLDAIEDDEQRSKTRARRTDNPPTPVDTLEEYKWRTRNKWDPAGCSTPLTGE |
| | GAILARKLSNACKKNLLTMTNILHNQKDLESFLQQNQQRESSESPKKARIQRKKGR |
| | KPLPKSRRRRRQYSSSSDDSESNSSSSSSSNSSPEKCSKRKRVST (SEQ ID |
| | NO: 11) |
| ORF2/3 | MSWRPPVHDAPGIERNWYEACFRAHAGACGCGNFIMHLNLLAGRYGFTPGSAPPG |
| | GPPPGTPQIRRARPSPAAPEQPAALPWHGDGGDGGAAGPPDAGGDAVAGAPYGEQ |
| | ELADLLDAIEDDEHRGRVPRARKRLVFRGRKVASLCRRADAGGDSTPPQATTQRAT |
| | AAPAAAPIPHPRNVQNASGSPPKPYVIKPAINQVYLFPERAPKPPPSSQDWQQEYEA |
| | CAAWDRPPRYNLSSPPFYPSCPSKFCVKFSLGFK (SEQ ID NO: 12) |
| ORF2t/3 | MSWRPPVHDAPGIERNCRGRVPRARKRLVFRGRKVASLCRRADAGGDSTPPQATT |
| | QRATAAPAAAPIPHPRNVQNASGSPPKPYVIKPAINQVYLFPERAPKPPPSSQDWQQ |
| | EYEACAAWDRPPRYNLSSPPFYPSCPSKFCVKFSLGFK (SEQ ID NO: 13) |
| ORF1 | MAWGWWRWRRRWPARRWRRRRRRRPVRRTRARRPARRYRRRRTVRTRRRRWG |
| | RRRYRRGWRRRTYVRKGRHRKKKKRLILRQWQPATRRRCTITGYLPIVFCGHTRG |
| | NKNYALHSDDYTPQGQPFGGALSTTSFSLKVLFDQHQRGLNKWSFPNDQLDLARY |
| | RGCKFIFYRTKQTDWVGQYDISEPYKLDKYSCPNYHPGNMIKAKHKFLIPSYDTNP |
| | RGRQKIIVKIPPPDLFVDKWYTQEDLCSVNLVSLAVSAASFLHPFGSPQTDNPCYTF |
| | QVLKEFYYQAIGFSASTQAMTSVLDTLYTQNSYWESNLTQFYVLNAKKGSDTTQPL |
| | TSNMPTREEFMAKKNTNYNWYTYKAASVKNKLHQMRQTYFEELTSKGPQTTKSE |
| | EGYSQHWTTPSTNAYEYHLGMFSAIFLAPDRPVPRFPCAYQDVTYNPLMDKGVGN |
| | HIWFQYNTKADTQLIVTGGSCKAHIQDIPLWAAFYGYSDFIESELGPFVDAETVGLV |
| | CVICPYTKPPMYNKTNPAMGYVFYDRNFGDGKWTDGRGKIEPYWQVRWRPEMLF |
| | QETVMADLVQTGPFSYKDELKNSTLVCKYKFYFTWGGNMMFQQTIKNPCKTDGQ |
| | PTDSSRHPRGIQVADPEQMGPRWVFHSFDWRRGYLSEKALKRLQEKPLDYDEYFT |
| | QPKRPRIFPPTESAEGEFREPEKGSYSEEERSQASAEEQTQEATVLLLKRRLREQQQL |
| | QQQLQFLTREMFKTQAGLHLNPMLLNQR (SEQ ID NO: 14) |
| ORF1/1 | MAWGWWRWRRRWPARRWRRRRRRRPVRRTRARRPARRYRRRRTTIKNPCKTDG |
| | QPTDSSRHPRGIQVADPEQMGPRWVFHSFDWRRGYLSEKALKRLQEKPLDYDEYF |
| | TQPKRPRIFPPTESAEGEFREPEKGSYSEEERSQASAEEQTQEATVLLLKRRLREQQQ |
| | LQQQLQFLTREMFKTQAGLHLNPMLLNQR (SEQ ID NO: 15) |
| ORF1/2 | MAWGWWRWRRRWPARRWRRRRRRRPVRRTRARRPARRYRRRRTQRESSESPKK |
| | ARIQRKKGRKPLPKSRRRRRQYSSSSDDSESNSSSSSSSNSSPEKCSKRKRVST (SEQ |
| | ID NO: 16) |

TABLE 5
Exemplary Anellovirus nucleic acid sequence
(Alphatorquevirus, Clade 3)

| | |
|---|---|
| Name | TTV-tth8 |
| Genus/Clade | Alphatorquevirus , Clade 3 |
| Accession Number | AJ620231.1 |
| Full Sequence: | 3753 bp |

1 10 20 30 40 50
| | | | | |
TGCTACGTCACTAACCCACGTGTCCTCTACAGGCCAATCGCAGTCTATGT
CGTGCACTTCCTGGGCATGGTCTACATAATTATATAAATGCTTGCACTTC
CGAATGGCTGAGTTTTTGCTGCCCGTCCGCGGAGAGGAGCCACGGCAGGG
GATCCGAACGTCCTGAGGGCGGGTGCCGGAGGTGAGTTTACACACCGAAG
TCAAGGGGCAATTCGGGCTCAGGACTGGCCGGGCTTTGGGCAAGGCTCTT
AAAAATGCACTTTTCTCGAATAAGCAGAAAGAAAAGGAAAGTGCTACTGC
TTTGCGTGCCAGCAGCTAAGAAAAAACCAACTGCTATGAGCTTCTGGAAA
CCTCCGGTACACAATGTCACGGGGATCCAACGCATGTGGTATGAGTCCTT
TCACCGTGGCCACGCTTCTTTTTGTGGTTGTGGGAATCCTATACTTCACA
TTACTGCACTTGCTGAAACATATGGCCATCCAACAGGCCCGAGACCTTCT
GGGCCACCGGGAGTAGACCCCAACCCCCACATCCGTAGAGCCAGGCCTGC
CCCGGCCGCTCCGGAGCCCTCACAGGTTGATTCGAGACCAGCCCTGACAT
GGCATGGGGATGGTGGAAGCGACGGAGGCGCTGGTGGTTCCGGAAGCGGT
GGACCCGTGGCAGACTTCGCAGACGATGGCCTCGATCAGCTCGTCGCCGC
CCTAGACGACGAAGAGTAAGGAGGCGCAGACGGTGGAGGAGGGGGAGACG
AAAAACAAGGACTTACAGACGCAGGAGACGCTTTAGACGCAGGGGACGAA
AAGCAAAACTTATAATAAAACTGTGGCAACCTGCAGTAATTAAAAGATGC
AGAATAAAGGGATACATACCACTGATTATAAGTGGGAACGGTACCTTTGC
CACAAACTTTACCAGTCACATAAATGACAGAATAATGAAAGGCCCCTTCG
GGGGAGGACACAGCACTATGAGGTTCAGCCTCTACATTTTGTTTGAGGAG
CACCTCAGACACATGAACTTCTGGACCAGAAGCAACGATAACCTAGAGCT
AACCAGATACTTGGGGGCTTCAGTAAAAATATACAGGCACCCAGACCAAG
ACTTTATAGTAATATACAACAGAAGAACCCCTCTAGGAGGCAACATCTAC
ACAGCACCCTCTCTACACCCAGGCAATGCCATTTTAGCAAAACACAAAAT
ATTAGTACCAAGTTTACAGACAAGACCAAAGGGTAGAAAAGCAATTAGAC
TAAGAATAGCACCCCCCACACTCTTTACAGACAAGTGGTACTTTCAAAAG
GACATAGCCGACCTCACCCTTTTCAACATCATGGCAGTTGAGGCTGACTT
GCGGTTTCCGTTCTGCTCACCACAAACTGACAACACTTGCATCAGCTTCC
AGGTCCTTAGTTCCGTTTACAACAACTACCTCAGTATTAATACCTTTAAT
AATGACAACTCAGACTCAAAGTTAAAAGAATTTTTAAATAAAGCATTTCC
AACAACAGGCACAAAAGGAACAAGTTTAAATGCACTAAATACATTTAGAA
CAGAAGGATGCATAAGTCACCCACAACTAAAAAAACCAAACCCACAAATA
AACAAACCATTAGAGTCACAATACTTTGCACCTTTAGATGCCCTCTGGGG
AGACCCCATATACTATAATGATCTAAATGAAAACAAAAGTTTGAACGATA
TCATTGAGAAAATACTAATAAAAAACATGATTACATACCATGCAAAACTA
AGAGAATTTCCAAATTCATACCAAGGAAACAAGGCCTTTTGCCACCTAAC
AGGCATATACAGCCCACCATACCTAAACCAAGGCAGAATATCTCCAGAAA
TATTTGGACTGTACACAGAAATAATTTACAACCCTTACACAGACAAAGGA
ACTGGAAACAAAGTATGGATGGACCCACTAACTAAAGAGAACAACATATA
TAAAGAAGGACAGAGCAAATGCCTACTGACTGACATGCCCCTATGGACTT
TACTTTTTGGATATACAGACTGGTGTAAAAAGGACACTAATAACTGGGAC
TTACCACTAAACTACAGACTAGTACTAATATGCCCTTATACCTTTCCAAA
ATTGTACAATGAAAAAGTAAAAGACTATGGGTACATCCCGTACTCCTACA
AATTCGGAGCGGGTCAGATGCCAGACGGCAGCAACTACATACCCTTTCAG
TTTAGAGCAAAGTGGTACCCCACAGTACTACACCAGCAACAGGTAATGGA
GGACATAAGCAGGAGCGGGCCCTTTGCACCTAAGGTAGAAAAACCAAGCA
CTCAGCTGGTAATGAAGTACTGTTTTAACTTTAACTGGGGCGGTAACCCT
ATCATTGAACAGATTGTTAAAGACCCCAGCTTCCAGCCCACCTATGAAAT
ACCCGGTACCGGTAACATCCCTAGAAGAATACAAGTCATCGACCCGCGGG
TCCTGGGACCGCACTACTCGTTCCGGTCATGGGACATGCGCAGACACACA
TTTAGCAGAGCAAGTATTAAGAGAGTGTCAGAACAACAAGAAACTTCTGA
CCTTGTATTCTCAGGCCCAAAAAAGCCTCGGGTCGACATCCCAAAACAAG
AAACCCAAGAAGAAAGCTCACATTCACTCCAAAGAGAATCGAGACCGTGG
GAGACCGAGGAAGAAAGCGAGACAGAAGCCCTCTCGCAAGAGAGCCAAGA
GGTCCCCTTCCAACAGCAGTTGCAGCAGCAGTACCAAGAGCAGCTCAAGC
TCAGACAGGGAATCAAAGTCCTCTTCGAGCAGCTCATAAGGACCCAACAA
GGGGTCCATGTAAACCCATGCCTACGGTAGGTCCCAGGCAGTGGCTGTTT
CCAGAGAGAAAGCCAGCCCCAGCTCCTAGCAGTGGAGACTGGGCCATGGA
GTTTCTCGCAGCAAAAATATTTGATAGGCCAGTTAGAAGCAACCTTAAAG
ATACCCCTTACTACCCATATGTTAAAAACCAATACAATGTCTACTTTGAC
CTTAAATTTGAATAAACAGCAGCTTCAAACTTGCAAGGCCGTGGGAGTTT
CACTGGTCGGTGTCTACCTCTAAAGGTCACTAAGCACTCCGAGCGTAAGC
GAGGAGTGCGACCCTCCCCCCTGGAACAACTTCTTCGGAGTCCGGCGCTA
CGCCTTCGGCTGCGCCGGACACCTCAGACCCCCCCTCCACCCGAAACGCT
TGCGCGTTTCGGACCTTCGGCGTCGGGGGGGTCGGGAGCTTTATTAAACG
GACTCCGAAGTGCTCTTGGACACTGAGGGGGTGAACAGCAACGAAAGTGA
GTGGGGCCAGACTTCGCCATAAGGCCTTTATCTTCTTGCCATTTGTCAGT
GTCCGGGGTCGCCATAGGCTTCGGGCTCGTTTTTAGGCCTTCCGGACTAC
AAAAATCGCCATTTTGGTGACGTCACGGCCGCCATCTTAAGTAGTTGAGG
CGGACGGTGGCGTGAGTTCAAAGGTCACCATCAGCCACACCTACTCAAAA
TGGTGGACAATTTCTTCCGGGTCAAAGGTTACAGCCGCCATGTTAAAACA
CGTGACGTATGACGTCACGGCCGCCATTTTGTGACACAAGATGGCCGACT
TCCTTCCTCTTTTTCAAAAAAAAGCGGAAGTGCCGCCGCGGCGGCGGGGG
GCGGCGCGCTGCGCGCGCCGCCCAGTAGGGGGAGCCATGCGCCCCCCCCC
GCGCATGCGCGGGGCCCCCCCCCGCGGGGGGCTCCGCCCCCCGGCCCCCC
CCG (SEQ ID NO: 17)

| | |
|---|---|
| Annotations: | |
| Putative Domain | Base range |
| TATA Box | 83-88 |
| Cap Site | 104-111 |
| Transcriptional Start Site | 111 |
| 5′ UTR Conserved Domain | 170-240 |
| ORF2 | 336-719 |
| ORF2/2 | 336-715; 2363-2789 |
| ORF2/3 | 336-715; 2565-3015 |
| ORF2t/3 | 336-388; 2565-3015 |
| ORF1 | 599-2830 |
| ORF1/1 | 599-715; 2363-2830 |
| ORF1/2 | 599-715; 2565-2789 |
| Three open-reading frame region | 2551-2786 |
| Poly(A) Signal | 3011-3016 |
| GC-rich region | 3632-3753 |

TABLE 6
Exemplary Anellovirus amino acid sequences (Alphatorquevirus, Clade 3)
TTV-tth8 (Alphatorquevirus Clade 3)

| | |
|---|---|
| ORF2 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEE (SEQ ID NO: 18) |
| ORF2/2 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEELLKTPASSPPMKYPVPVTSLEEYKSSTRGSWDRTTRSGHGT |
| | CADTHLAEQVLRECQNNKKLLTLYSQAQKSLGSTSQNKKPKKKAHIHSKENRDRG |
| | RPRKKARQKPSRKRAKRSPSNSSCSSSTKSSSSSDRESKSSSSSS (SEQ ID NO: 19) |
| ORF2/3 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEEPKKASGRHPKTRNPRRKLTFTPKRIETVGDRGRKRDRSPLA |
| | REPRGPLPTAVAAAVPRAAQAQTGNQSPLRAAHKDPTRGPCKPMPTVGPRQWLFP |
| | ERKPAPAPSSGDWAMEFLAAKIFDRPVRSNLKDTPYYPYVKNQYNVYFDLKFE |
| | (SEQ ID NO: 20) |
| ORF2t/3 | MSFWKPPVHNVTGIQRMWPKKASGRHPKTRNPRRKLTFTPKRIETVGDRGRKRDR |
| | SPLAREPRGPLPTAVAAAVPRAAQAQTGNQSPLRAAHKDPTRGPCKPMPTVGPRQ |
| | WLFPERKPAPAPSSGDWAMEFLAAKIFDRPVRSNLKDTPYYPYVKNQYNVYFDLK |
| | FE (SEQ ID NO: 21) |
| ORF1 | MAWGWWKRRRRWWFRKRWTRGRLRRRWPRSARRRPRRRRVRRRRRWRRGRRK |
| | TRTYRRRRRFRRRGRKAKLIIKLWQPAVIKRCRIKGYIPLIISGNGTFATNFTSHINDR |
| | IMKGPFGGGHSTMRFSLYILFEEHLRHMNFWTRSNDNLELTRYLGASVKIYRHPDQ |
| | DFIVIYNRRTPLGGNIYTAPSLHPGNAILAKHKILVPSLQTRPKGRKAIRLRIAPPTLFT |
| | DKWYFQKDIADLTLFNIMAVEADLRFPFCSPQTDNTCISFQVLSSVYNNYLSINTFN |
| | NDNSDSKLKEFLNKAFPTTGTKGTSLNALNTFRTEGCISHPQLKKPNPQINKPLESQ |
| | YFAPLDALWGDPIYYNDLNENKSLNDIIEKILIKNMITYHAKLREFPNSYQGNKAFC |
| | HLTGIYSPPYLNQGRISPEIFGLYTEIIYNPYTDKGTGNKVWMDPLTKENNIYKEGQS |
| | KCLLTDMPLWTLLFGYTDWCKKDTNNWDLPLNYRLVLICPYTFPKLYNEKVKDY |
| | GYIPYSYKFGAGQMPDGSNYIPFQFRAKWYPTVLHQQQVMEDISRSGPFAPKVEKP |
| | STQLVMKYCFNFNWGGNPIIEQIVKDPSFQPTYEIPGTGNIPRRIQVIDPRVLGPHYSF |
| | RSWDMRRHTFSRASIKRVSEQQETSDLVFSGPKKPRVDIPKQETQEESSHSLQRESR |
| | PWETEEESETEALSQESQEVPFQQQLQQQYQEQLKLRQGIKVLFEQLIRTQQGVHV |
| | NPCLR (SEQ ID NO: 22) |
| ORF1/1 | MAWGWWKRRRRWWFRKRWTRGRLRRRWPRSARRRPRRRRIVKDPSFQPTYEIPG |
| | TGNIPRRIQVIDPRVLGPHYSFRSWDMRRHTFSRASIKRVSEQQETSDLVFSGPKKPR |
| | VDIPKQETQEESSHSLQRESRPWETEEESETEALSQESQEVPFQQQLQQQYQEQLKL |
| | RQGIKVLFEQLIRTQQGVHVNPCLR (SEQ ID NO: 23) |
| ORF1/2 | MAWGWWKRRRRWWFRKRWTRGRLRRRWPRSARRRPRRRRAQKSLGSTSQNKK |
| | PKKKAHIHSKENRDRGRPRKKARQKPSRKRAKRSPSNSSCSSSTKSSSSSDRESKSSS |
| | SSS (SEQ ID NO: 24) |

TABLE 7
Exemplary Anellovirus nucleic acid sequence
(Alphatorquevirus, Clade 4)

| | |
|---|---|
| Name | TTV-JA20 |
| Genus/Clade | Alphatorquevirus, Clade 4 |
| Accession Number | AF122914.3 |
| Full Sequence: | 3853 bp |

1 10 20 30 40 50
| | | | | |
GGCTTAGTGCGTCACCACCCACGTGACCCGCCTCCGCCAATTAACAGGTA
CTTCGTACACTTCCTGGGCGGGCTTATAAGACTAATATAAGTAGCTGCAC
TTCCGAATGGCTGAGTTTTCCACGCCCGTCCGCAGCGGTGAAGCCACGGA
GGGAGCTCAGCGCGTCCCGAGGGCGGGTGCCGGAGGTGAGTTTACACACC
GCAGTCAAGGGGCAATTCGGGCTCGGGACTGGCCGGGCTTTGGGCAAGGC
TCTTAAAAAAGCTATGTTTATTGGCAGGCACTACCGAAAGAAAAGGGCGC
TGCTACTGCTATCTGTGCATTCTACAAAGACAAAAGGGAAACTTCTAATA
GCTATGTGGACTCCCCCACGCAATGATCAACAATACCTTAACTGGCAATG
GTACACTTCTGTACTTAGCTCCCACTCTGCTATGTGCGGGTGTTCCGACG
CTATCGCTCATCTTAATCATCTTGCTAATCTGCTTCGTGCCCCGCAAAAT
CCGCCCCCGCCTGATAATCCAAGACCCCTACCCGTGCGAGCACTGCCTGC
TCCCCCGGCTGCCCACGAGGCAGCCGGTGATCGAGCACCATGGCCTATGG
GTGGTGGAGGAGACGCCGGAGGCGCTGGCGCAGGTGGAGACGCCGACCAT
GGAGGCGCCGCTGGAGGACCCGCAGACGCAGACCTGCTAGACGCCGTGGC
CGCCGCAGAAACGTAAGGAGACGGCGCAGAGGGAGGTGGAGAAGGAGGTA
CAGGAGGTGGAAAAGAAAGGGCAGACGTAGAAGAAAAGCAAAAATAATAA
TAAGACAGTGGCAGCCAAACTACAGAAGAAGATGTAATATAGTGGGCTAC
CTCCCTATACTTATCTGTGGTGGAAATACTGTTTCTAGAAACTATGCCAC
ACACTCAGACGATACTAACTATCCAGGACCCTTTGGGGGAGGCATGACCA
CAGACAAATTCAGCCTTAGAATACTATATGATGAATACAAAAGATTTATG
AACTACTGGACAGCCTCAAATGAGGACCTAGATCTCTGTAGATATCTAGG
ATGCACTTTTTACTTCTTTAGACACCCTGAAGTAGACTTTATTATAAAAA
TAAACACCATGCCCCCATTCTTAGATACAACCATAACAGCACCTAGCATA
CACCCAGGCCTCATGGCCCTAGACAAAAGAGCCAGATGGATTCCTTCTCT
TAAAAATAGACCAGGTAAAAAACACTATATAAAAATTAGAGTAGGGGCTC
CTAAAATGTTCACAGATAAATGGTACCCTCAAACAGACCTCTGTGACATG
ACACTGCTAACTATCTATGCAACCGCAGCGGATATGCAATATCCGTTCGG
CTCACCACTAACTGACACTGTGGTTGTTAACTCCCAAGTTCTGCAATCCA
TGTATGATGAAACAATTAGCATATTACCTGATGAAAAAACTAAAAGAAAT
AGCCTTCTTACTTCTATAAGAAGCTACATACCTTTTTATAATACTACACA
AACAATAGCTCAATTAAAACCATTTGTAGATGCAGGAGGACACACAACAG
GCTCAACAACAACTACATGGGGACAACTATTAAACACAACTAAATTTACC
ACTACCACAACAACCACATACACATACCCTGGCACCACAAATACAGCAGT
AACATTTATAACAGCCAATGATACCTGGTACAGGGGAACAGCATATAAAG
ATAACATTAAAGATGTACCACAAAAAGCAGCACAATTATACTTTCAAACA
ACACAAAAACTACTAGGAAACACATTCCATGGCTCAGATGAAACACTTGA
ATACCATGCAGGCCTATACAGCTCTATCTGGCTATCACCAGGTAGATCCT
ACTTTGAAACACCAGGTGCATACACAGACATTAAATATAACCCTTTTACA
GACAGAGGAGAAGGCAACATGCTGTGGATAGACTGGCTAAGTAAAAAAAA
CATGAAATATGACAAAGTGCAAAGTAAGTGCCTAGTAGCAGACCTACCAC
TGTGGGCAGCAGCATATGGTTATGTAGAATTCTGCTCTAAAAGCACAGGA
GACACAAACATACACATGAATGCCAGACTACTAATAAGAAGTCCTTTTAC
AGACCCCCAGCTAATAGTACACACAGACCCCACTAAAGGCTTTGTACCCT
ATTCTTTAAACTTTGGAAATGGTAAAATGCCAGGAGGTAGCAGCAATGTT
CCCATAAGAATGAGAGCTAAGTGGTACCCCACTTTATCCCACCAACAAGA
AGTTCTAGAGGCCTTAGCACAGTCAGGACCCTTTGCTTATCACTCAGACA
TTAAAAAAGTATCTCTAGGCATAAAATACCGTTTTAAGTGGATCTGGGGT
GGAAACCCCGTTCGCCAACAGGTTGTTAGAAATCCCTGCAAGGAACCCCA
CTCCTCGGGCAATAGAGTCCCTAGAAGCATACAAATCGTTGACCCGAGAT
ACAACTCACCGGAACTTACCATCCATGCCTGGGACTTCAGACGTGGCTTC
TTTGGCCCGAAAGCTATTCAAAGAATGCAACAACAACCAACTGCTACTGA
ATTTTTTTCAGCAGGCCGCAAGAGACCCAGAAGGGACACAGAAGTGTATC
AGTCCGACCAAGAAAAGGAGCAAAAAGAAAGCTCGCTTTTCCCCCCAGTC
AAGCTCCTCCGAAGAGTCCCCCCGTGGGAGGACTCGGAACAGGAGCAAAG
CGGGTCGCAAAGCTCAGAGGAAGAGACGGCGACCCTCTCCCAGCAGCTCA
AACAGCAGCTGCAGCAGCAGCGAGTCTTGGGAGTCAAACTCAGACTCCTG
TTCAACCAAGTCCAAAAAATCCAACAAAATCAAGATATCAACCCTACCTT
GTTACCAAGGGGGGGGGATCTAGTATCCTTCTTTCAGGCTGTACCATAAA
TATGTTTCCAGACCCTAAACCTTACTGCCCCTCCAGCAATGACTGGAAAG
AAGAGTATGAGGCCTGTAAATATTGGGATAGACCTCCCAGACACAACCTT
AGAGACCCCCCCTTTTACCCCTGGGCCCCTAAAAACAATCCTTGCAATGT
AAGCTTTAAACTTGGCTTCAAATAAACTAGGCCGTGGGAGTTTCACTTGT
CGGTGTCTACCTCTATAAGTCACTAAGCACTCCGAGCGCAGCGAGGAGTG
CGACCCTTCCCCCTGGTGCAACGCCCTCGGCGGCCGCGCGCTACGCCTTC
GGCTGCGCGCGGCACCTCGGACCCCCGCTCGTGCTGACACGCTTGCGCGT
GTCAGACCACTTCGGGCTCGCGGGGGTCGGGAAATTTGCTAAACAGACTC
CGAGTTGCCATTGGACACTGTAGCTATGAATCAGTAACGAAAGTGAGTGG
GGCCAGACTTCGCCATAAGGCCTTTATCTTCTTGCCATTTGTCAGTATTG
GGGGTCGCCATAAACTTTGGGCTCCATTTTAGGCCTTCCGGACTACAAAA
ATCGCCATATTTGTGACGTCAGAGCCGCCATTTTAAGTCAGCTCTGGGGA
GGCGTGACTTCCAGTTCAAAGGTCATCCTCACCATAACTGGCACAAAATG
GCCGCCAACTTCTTCCGGGTCAAAGGTCACTGCTACGTCATAGGTGACGT
GGGGGGGGACCTACTTAAACACGGAAGTAGGCCCCGACACGTCACTGTCA
CGTGACAGTACGTCACAGCCGCCATTTTGTTTTACAAAATAGCCGACTTC
CTTCCTCTTTTTTAAAAAAAGGCGCCAAAAAACCGTCGGCGGGGGGGCCG
CGCGCTGCGCGCGCGGCCCCCGGGGGAGGCACAGCCTCCCCCCCCCGCGC
GCATGCGCGCGGGTCCCCCCCCCTCCGGGGGGCTCCGCCCCCCGGCCCCC
CCC (SEQ ID NO: 25)
Annotations:
Putative Domain Base range
TATA Box 86-90
Cap Site 107-114
Transcriptional Start Site 114
5′ UTR Conserved Domain 174-244
ORF2 354-716
ORF2/2 354-712; 2372-2873
ORF2/3 354-712; 2565-3075
ORF2t/3 354-400; 2565-3075
ORF1 590-2899
ORF1/1 590-712; 2372-2899
ORF1/2 590-712; 2565-2873
Three open-reading frame region 2551-2870
Poly(A) Signal 3071-3076
GC-rich region 3733-3853

TABLE 8
Exemplary Anellovirus amino acid sequences (Alphatorquevirus, Clade 4)
TTV-JA20 (Alphatorquevirus Clade 4)

| | |
|---|---|
| ORF2 | MWTPPRNDQQYLNWQWYTSVLSSHSAMCGCSDAIAHLNHLANLLRAPQNPPPPD |
| | NPRPLPVRALPAPPAAHEAAGDRAPWPMGGGGDAGGAGAGGDADHGGAAGGPA |
| | DADLLDAVAAAET (SEQ ID NO: 26) |
| ORF2/2 | MWTPPRNDQQYLNWQWYTSVLSSHSAMCGCSDAIAHLNHLANLLRAPQNPPPPD |
| | NPRPLPVRALPAPPAAHEAAGDRAPWPMGGGGDAGGAGAGGDADHGGAAGGPA |
| | DADLLDAVAAAETLLEIPARNPTPRAIESLEAYKSLTRDTTHRNLPSMPGTSDVASL |
| | ARKLFKECNNNQLLLNFFQQAARDPEGTQKCISPTKKRSKKKARFSPQSSSSEESPR |
| | GRTRNRSKAGRKAQRKRRRPSPSSSNSSCSSSESWESNSDSCSTKSKKSNKIKISTLP |
| | CYQGGGI (SEQ ID NO: 27) |
| ORF2/3 | MWTPPRNDQQYLNWQWYTSVLSSHSAMCGCSDAIAHLNHLANLLRAPQNPPPPD |
| | NPRPLPVRALPAPPAAHEAAGDRAPWPMGGGGDAGGAGAGGDADHGGAAGGPA |
| | DADLLDAVAAAETPQETQKGHRSVSVRPRKGAKRKLAFPPSQAPPKSPPVGGLGTG |
| | AKRVAKLRGRDGDPLPAAQTAAAAAASLGSQTQTPVQPSPKNPTKSRYQPYLVTK |
| | GGGSSILLSGCTINMFPDPKPYCPSSNDWKEEYEACKYWDRPPRHNLRDPPFYPWA |
| | PKNNPCNVSFKLGFK (SEQ ID NO: 28) |
| ORF2t/3 | MWTPPRNDQQYLNWQWPQETQKGHRSVSVRPRKGAKRKLAFPPSQAPPKSPPVG |
| | GLGTGAKRVAKLRGRDGDPLPAAQTAAAAAASLGSQTQTPVQPSPKNPTKSRYQP |
| | YLVTKGGGSSILLSGCTINMFPDPKPYCPSSNDWKEEYEACKYWDRPPRHNLRDPP |
| | FYPWAPKNNPCNVSFKLGFK (SEQ ID NO: 29) |
| ORF1 | MAYGWWRRRRRRWRRWRRRPWRRRWRTRRRRPARRRGRRRNVRRRRRGRWRR |
| | RYRRWKRKGRRRRKAKIIIRQWQPNYRRRCNIVGYLPILICGGNTVSRNYATHSDD |
| | TNYPGPFGGGMTTDKFSLRILYDEYKRFMNYWTASNEDLDLCRYLGCTFYFFRHPE |
| | VDFIIKINTMPPFLDTTITAPSIHPGLMALDKRARWIPSLKNRPGKKHYIKIRVGAPK |
| | MFTDKWYPQTDLCDMTLLTIYATAADMQYPFGSPLTDTVVVNSQVLQSMYDETISI |
| | LPDEKTKRNSLLTSIRSYIPFYNTTQTIAQLKPFVDAGGHTTGSTTTTWGQLLNTTKF |
| | TTTTTTTYTYPGTTNTAVTFITANDTWYRGTAYKDNIKDVPQKAAQLYFQTTQKLL |
| | GNTFHGSDETLEYHAGLYSSIWLSPGRSYFETPGAYTDIKYNPFTDRGEGNMLWID |
| | WLSKKNMKYDKVQSKCLVADLPLWAAAYGYVEFCSKSTGDTNIHMNARLLIRSPF |
| | TDPQLIVHTDPTKGFVPYSLNFGNGKMPGGSSNVPIRMRAKWYPTLSHQQEVLEAL |
| | AQSGPFAYHSDIKKVSLGIKYRFKWIWGGNPVRQQVVRNPCKEPHSSGNRVPRSIQI |
| | VDPRYNSPELTIHAWDFRRGFFGPKAIQRMQQQPTATEFFSAGRKRPRRDTEVYQS |
| | DQEKEQKESSLFPPVKLLRRVPPWEDSEQEQSGSQSSEEETATLSQQLKQQLQQQR |
| | VLGVKLRLLFNQVQKIQQNQDINPTLLPRGGDLVSFFQAVP (SEQ ID NO: 30) |
| ORF1/1 | MAYGWWRRRRRRWRRWRRRPWRRRWRTRRRRPARRRGRRRNVVRNPCKEPHSS |
| | GNRVPRSIQIVDPRYNSPELTIHAWDFRRGFFGPKAIQRMQQQPTATEFFSAGRKRP |
| | RRDTEVYQSDQEKEQKESSLFPPVKLLRRVPPWEDSEQEQSGSQSSEEETATLSQQL |
| | KQQLQQQRVLGVKLRLLFNQVQKIQQNQDINPTLLPRGGDLVSFFQAVP (SEQ ID |
| | NO: 31) |
| ORF1/2 | MAYGWWRRRRRRWRRWRRRPWRRRWRTRRRRPARRRGRRRNAARDPEGTQKCI |
| | SPTKKRSKKKARFSPQSSSSEESPRGRTRNRSKAGRKAQRKRRRPSPSSSNSSCSSSE |
| | SWESNSDSCSTKSKKSNKIKISTLPCYQGGGI (SEQ ID NO: 32) |

TABLE 9
Exemplary Anellovirus nucleic acid sequence
(Alphatorquevirus, Clade 5)

| | |
|---|---|
| Name | TTV-HD23a |
| Genus/Clade | Alphatorquevirus , Clade 5 |
| Accession Number | FR751500.1 |
| Full Sequence: | 3758 bp |

1 10 20 30 40 50
| | | | | |
AAAGTACGTCACTAACCACGTGACTCCCACAGGCCAACCACAGTCTACGT
CGTGCATTTCCTGGGCATGGTCTACATCATAATATAAGAAGGCGCACTTC
CGAATGGCTGAGTTTTCCACGCCCGTCCGCAGCGAGAACGCCACGGAGGG
AGATCCTCGCGTCCCGAGGGCGGGTGCCGGAGGTGAGTTTACACACCGCA
GTCAAGGGGCAATTCGGGCTCGGGACTGGCCGGGCCCTGGGCAAGGCTCT
TAAAAAATGCGCTTTCGCAGGGTTGCGGAGAAAAGGAAAGTGCTTCTGCA
AACTCTGCGAGCTGCAAAGCAGGCTAGGCGGCTTCTAGGTATGTGGCAGC
CCCCCGCGCACAATGTCCCCGGCATCGAGAGAAACTGGTACGAGAGCTGC
TTCAGGTCTCACGCTGCTGTTTGTGGCTGTGGCGACTTTGTTGGCCATAT
TAATCATTTGGCAACTACTCTGGGTCGTCCTCCGCGTCCTGGGCCCCCAG
GCGGACCCCGCACGCCGCAAATAAGAAACCTGCCAGCGCTCCCGGCGCCC
CAGGGCGAGCCCGGTGACAGAGCGCCATGGCGTGGGGTTTCTGGGGCCGA
CGCCGCCGGTGGAGACGGTGGAGAGCGCGGCGCAGACGGTGGAGACCCCG
GAGACGTAGGAGACGACGCCCTGCTCGCCGCTTTCGAGCTCGTCGAAGAG
TAAGGAGACGCGGGGGGAGGTGGCGCAGACGCTACAGAAAATGGCGACGG
GGCAGACGCAGACGGACTCACAGAAAAAAGATAATTATAAAACAGTGGCA
ACCAAACTTTATTAGACGCTGCTACATAATAGGATGCCTACCTCTCGTTT
TCTGTGGCGAAAATACAACCGCCCAGAACTATGCCACTCACTCAGACGAT
CCCCCCCC (SEQ ID NO: 33)
Annotations:
Putative Domain Base range
TATA Box 83-87
Cap Site 104-111
Transcriptional Start Site 111
5′ UTR Conserved Domain 171-241
ORF2 341-703
ORF2/2 341-699; 2311-2806
ORF2/3 341-699; 2504-2978
ORF2t/3 341-387; 2504-2978
ORF1 577-2787-2787
ORF1/1 577-699; 2311
ORF1/2 577-699; 2504-2806
Three open-reading frame region 2463-2784
Poly(A) Signal 2974-2979
GC-rich region 3644-3758

TABLE 10
Exemplary Anellovirus amino acid sequences (Alphatorquevirus, Clade 5)
TTV-HD23a (Alphatorquevirus Clade 5)

| | |
|---|---|
| ORF2 | MWQPPAHNVPGIERNWYESCFRSHAAVCGCGDFVGHINHLATTLGRPPRPGPPGGP |
| | RTPQIRNLPALPAPQGEPGDRAPWRGVSGADAAGGDGGERGADGGDPGDVGDDA |
| | LLAAFELVEE (SEQ ID NO: 34) |
| ORF2/2 | MWQPPAHNVPGIERNWYESCFRSHAAVCGCGDFVGHINHLATTLGRPPRPGPPGGP |
| | RTPQIRNLPALPAPQGEPGDRAPWRGVSGADAAGGDGGERGADGGDPGDVGDDA |
| | LLAAFELVEESSGIPAPTPAPPRPIEDLAAYKRLTRNTIPQKSLGTRGTSDEDSLAKQ |
| | VLKECNKNQMLFTFLQDHSRGLAETPTPKTRKSKTKAHVSESSSDSRGSTPAKRRK |
| | APKKRRRRRGRYKTNYSSSSESSEYSDSSSNNSHPKSSKFKQDTAYTPYYPPKHKQS |
| | LYV (SEQ ID NO: 35) |
| ORF2/3 | MWQPPAHNVPGIERNWYESCFRSHAAVCGCGDFVGHINHLATTLGRPPRPGPPGGP |
| | RTPQIRNLPALPAPQGEPGDRAPWRGVSGADAAGGDGGERGADGGDPGDVGDDA |
| | LLAAFELVEETTQEASQRHQRPRPGKAKRKLTFRSPAATPVGPLQPRDAKLRRRDA |
| | GAGVGTRPTTPPAPRAASTPTPAPTTRTPSPQSSSRTQPTPPIILPSINKAYMFEPQGPK |
| | PIQGYNDWLEEYTSCKFRDRPPRMLHTDLPFYPWAPKPQDQVRVTFKLNFQ (SEQ |
| | ID NO: 36) |
| ORF2t/3 | MWQPPAHNVPGIERNWTTQEASQRHQRPRPGKAKRKLTFRSPAATPVGPLQPRDA |
| | KLRRRDAGAGVGTRPTTPPAPRAASTPTPAPTTRTPSPQSSSRTQPTPPIILPSINKAY |
| | MFEPQGPKPIQGYNDWLEEYTSCKFRDRPPRMLHTDLPFYPWAPKPQDQVRVTFKL |
| | NFQ (SEQ ID NO: 37) |
| ORF1 | MAWGFWGRRRRWRRWRARRRRWRPRRRRRRRPARRFRARRRVRRRGGRWRRR |
| | YRKWRRGRRRRTHRKKIIIKQWQPNFIRRCYIIGCLPLVFCGENTTAQNYATHSDDM |
| | ISKGPYGGGMTTTKFTLRILYDEFTRFMNFWTVSNEDLDLCRYVGCKLIFFKHPTVD |
| | FMVQINTQPPFLDTSLTAASIHPGIMMLSKRRILIPSLKTRPSRKHRVVVRVGAPRLF |
| | QDKWYPQSDLCDTVLLSIFATARDLQYPFGSPLTDNPCVNFQILGPQYKKHLSISST |
| | MDDTNKQHYNSNLFNKTALYNTFQTIARLKETGQTANISPSWSEVQNTKLLDHTG |
| | ANATASRDTWYKGNTYNDYIQQLAEKTRERFKKATMSALPNYPTIMSTDLYEYHS |
| | GIYSSIFLSAGRSYFETTGAYSDITYNPLTDKGTGNIIWIDYLTKDDTIFVKNKSKCEI |
| | MDMPLWAAGTGYTEFCAKYTGDSAIIYNARILIRCPYTEPMLIDHSDPNKGFVPYSF |
| | NFGNGKMPGGSSNVPIRMRAKWYVNIFHQKEVLESIVQSGPFGYRGDIKSAVLSMK |
| | YRFHWKWGGNPISKQVVRNPCSNSSTSAAHRGPRSVQAVDPKYNTPEVTWHSWDI |
| | RRGLFGKAGIKRMQQESDALYVPAGPLKRPRRDTNAQDPEKQNESSRFGVQQRLP |
| | WVHSSQETQSSEEETQAQGSVQDQLLLQLREQRVLRLQLQQLAPQVLKVQAGHSL |
| | HPLLSSQA (SEQ ID NO: 38) |
| ORF1/1 | MAWGFWGRRRRWRRWRARRRRWRPRRRRRRRPARRFRARRRVVRNPCSNSSTS |
| | AAHRGPRSVQAVDPKYNTPEVTWHSWDIRRGLFGKAGIKRMQQESDALYVPAGPL |
| | KRPRRDTNAQDPEKQNESSRFGVQQRLPWVHSSQETQSSEEETQAQGSVQDQLLLQ |
| | LREQRVLRLQLQQLAPQVLKVQAGHSLHPLLSSQA (SEQ ID NO: 39) |
| ORF1/2 | MAWGFWGRRRRWRRWRARRRRWRPRRRRRRRPARRFRARRRDHSRGLAETPTP |
| | KTRKSKTKAHVSESSSDSRGSTPAKRRKAPKKRRRRRGRYKTNYSSSSESSEYSDSS |
| | SNNSHPKSSKFKQDTAYTPYYPPKHKQSLYV (SEQ ID NO: 40) |

TABLE 11
Exemplary Anellovirus nucleic acid sequence
(Betatorquevirus)

| | |
|---|---|
| Name | TTMV-LY2 |
| Genus/Clade | Betatorquevirus |
| Accession Number | JX134045.1 |
| Full Sequence: | 2797 bp |

1 10 20 30 40 50
| | | | | |
TAATAAATATTCAACAGGAAAACCACCTAATTTAAATTGCCGACCACAAA
CCGTCACTTAGTTCCCCTTTTTGCAACAACTTCTGCTTTTTTCCAACTGC
CGGAAAACCACATAATTTGCATGGCTAACCACAAACTGATATGCTAATTA
ACTTCCACAAAACAACTTCCCCTTTTAAAACCACACCTACAAATTAATTA
TTAAACACAGTCACATCCTGGGAGGTACTACCACACTATAATACCAAGTG
CACTTCCGAATGGCTGAGTTTATGCCGCTAGACGGAGAACGCATCAGTTA
CTGACTGCGGACTGAACTTGGGCGGGTGCCGAAGGTGAGTGAAACCACCG
AAGTCAAGGGGCAATTCGGGCTAGTTCAGTCTAGCGGAACGGGCAAGAAA
CTTAAAATTATTTTATTTTTCAGATGAGCGACTGCTTTAAACCAACATGC
TACAACAACAAAACAAAGCAAACTCACTGGATTAATAACCTGCATTTAAC
CCACGACCTGATCTGCTTCTGCCCAACACCAACTAGACACTTATTACTAG
CTTTAGCAGAACAACAAGAAACAATTGAAGTGTCTAAACAAGAAAAAGAA
AAAATAACAAGATGCCTTATTACTACAGAAGAAGACGGTACAACTACAGA
CGTCCTAGATGGTATGGACGAGGTTGGATTAGACGCCCTTTTCGCAGAAG
ATTTCGAAGAAAAAGAAGGGTAAGACCTACTTATACTACTATTCCTCTAA
AGCAATGGCAACCGCCATATAAAAGAACATGCTATATAAAAGGACAAGAC
TGTTTAATATACTATAGCAACTTAAGACTGGGAATGAATAGTACAATGTA
TGAAAAAAGTATTGTACCTGTACATTGGCCGGGAGGGGGTTCTTTTTCTG
TAAGCATGTTAACTTTAGATGCCTTGTATGATATACATAAACTTTGTAGA
AACTGGTGGACATCCACAAACCAAGACTTACCACTAGTAAGATATAAAGG
ATGCAAAATAACATTTTATCAAAGCACATTTACAGACTACATAGTAAGAA
TACATACAGAACTACCAGCTAACAGTAACAAACTAACATACCCAAACACA
CATCCACTAATGATGATGATGTCTAAGTACAAACACATTATACCTAGTAG
ACAAACAAGAAGAAAAAAGAAACCATACACAAAAATATTTGTAAAACCAC
CTCCGCAATTTGAAAACAAATGGTACTTTGCTACAGACCTCTACAAAATT
CCATTACTACAAATACACTGCACAGCATGCAACTTACAAAACCCATTTGT
AAAACCAGACAAATTATCAAACAATGTTACATTATGGTCACTAAACACCA
TAAGCATACAAAATAGAAACATGTCAGTGGATCAAGGACAATCATGGCCA
TTTAAAATACTAGGAACACAAAGCTTTTATTTTTACTTTTACACCGGAGC
AAACCTACCAGGTGACACAACACAAATACCAGTAGCAGACCTATTACCAC
TAACAAACCCAAGAATAAACAGACCAGGACAATCACTAAATGAGGCAAAA
ATTACAGACCATATTACTTTCACAGAATACAAAAACAAATTTACAAATTA
TTGGGGTAACCCATTTAATAAACACATTCAAGAACACCTAGATATGATAC
TATACTCACTAAAAAGTCCAGAAGCAATAAAAAACGAATGGACAACAGAA
AACATGAAATGGAACCAATTAAACAATGCAGGAACAATGGCATTAACACC
ATTTAACGAGCCAATATTCACACAAATACAATATAACCCAGATAGAGACA
CAGGAGAAGACACTCAATTATACCTACTCTCTAACGCTACAGGAACAGGA
TGGGACCCACCAGGAATTCCAGAATTAATACTAGAAGGATTTCCACTATG
GTTAATATATTGGGGATTTGCAGACTTTCAAAAAAACCTAAAAAAAGTAA
CAAACATAGACACAAATTACATGTTAGTAGCAAAAACAAAATTTACACAA
AAACCTGGCACATTCTACTTAGTAATACTAAATGACACCTTTGTAGAAGG
CAATAGCCCATATGAAAAACAACCTTTACCTGAAGACAACATTAAATGGT
ACCCACAAGTACAATACCAATTAGAAGCACAAAACAAACTACTACAAACT
GGGCCATTTACACCAAACATACAAGGACAACTATCAGACAATATATCAAT
GTTTTATAAATTTTACTTTAAATGGGGAGGAAGCCCACCAAAAGCAATTA
ATGTTGAAAATCCTGCCCACCAGATTCAATATCCCATACCCCGTAACGAG
CATGAAACAACTTCGTTACAGAGTCCAGGGGAAGCCCCAGAATCCATCTT
ATACTCCTTCGACTATAGACACGGGAACTACACAACAACAGCTTTGTCAC
GAATTAGCCAAGACTGGGCACTTAAAGACACTGTTTCTAAAATTACAGAG
CCAGATCGACAGCAACTGCTCAAACAAGCCCTCGAATGCCTGCAAATCTC
GGAAGAAACGCAGGAGAAAAAAGAAAAAGAAGTACAGCAGCTCATCAGCA
ACCTCAGACAGCAGCAGCAGCTGTACAGAGAGCGAATAATATCATTATTA
AAGGACCAATAACTTTTAACTGTGTAAAAAAGGTGAAATTGTTTGATGAT
AAACCAAAAAACCGTAGATTTACACCTGAGGAATTTGAAACTGAGTTACA
AATAGCAAAATGGTTAAAGAGACCCCCAAGATCCTTTGTAAATGATCCTC
CCTTTTACCCATGGTTACCACCTGAACCTGTTGTAAACTTTAAGCTTAAT
TTTACTGAATAAAGGCCAGCATTAATTCACTTAAGGAGTCTGTTTATTTA
AGTTAAACCTTAATAAACGGTCACCGCCTCCCTAATACGCAGGCGCAGAA
AGGGGGCTCCGCCCCCTTTAACCCCCAGGGGGCTCCGCCCCCTGAAACCC
CCAAGGGGGCTACGCCCCCTTACACCCCC (SEQ ID NO: 41)
Annotations:
Putative Domain Base range
TATA Box 237-243
Cap Site 260-267
Transcriptional Start Site 267
5′ UTR Conserved Domain 323-393
ORF2 424-723
ORF2/2 424-719; 2274-2589
ORF2/3 424-719; 2449-2812
ORF1 612-2612
ORF1/1 612-719; 2274-2612
ORF1/2 612-719; 2449-2589
Three open-reading frame region 2441-2586
Poly(A) Signal 2808-2813
GC-rich region 2868-2929

TABLE 12
Exemplary Anellovirus amino acid sequences (Betatorquevirus)
TTMV-LY2 (Betatorquevirus)

| | |
|---|---|
| ORF2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEG (SEQ ID NO: 42) |
| ORF2/2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGFNIPYPVTSMKQLRY |
| | RVQGKPQNPSYTPSTIDTGTTQQQLCHELAKTGHLKTLFLKLQSQIDSNCSNKPSNA |
| | CKSRKKRRRKKKKKYSSSSATSDSSSSCTESE (SEQ ID NO: 43) |
| ORF2/3 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGARSTATAQTSPRMP |
| | ANLGRNAGEKRKRSTAAHQQPQTAAAAVQRANNIIIKGPITFNCVKKVKLFDDKPK |
| | NRRFTPEEFETELQIAKWLKRPPRSFVNDPPFYPWLPPEPVVNFKLNFTE (SEQ ID |
| | NO: 44) |
| ORF1 | MPYYYRRRRYNYRRPRWYGRGWIRRPFRRRFRRKRRVRPTYTTIPLKQWQPPYKR |
| | TCYIKGQDCLIYYSNLRLGMNSTMYEKSIVPVHWPGGGSFSVSMLTLDALYDIHKL |
| | CRNWWTSTNQDLPLVRYKGCKITFYQSTFTDYIVRIHTELPANSNKLTYPNTHPLM |
| | MMMSKYKHIIPSRQTRRKKKPYTKIFVKPPPQFENKWYFATDLYKIPLLQIHCTACN |
| | LQNPFVKPDKLSNNVTLWSLNTISIQNRNMSVDQGQSWPFKILGTQSFYFYFYTGA |
| | NLPGDTTQIPVADLLPLTNPRINRPGQSLNEAKITDHITFTEYKNKFTNYWGNPFNK |
| | HIQEHLDMILYSLKSPEAIKNEWTTENMKWNQLNNAGTMALTPFNEPIFTQIQYNP |
| | DRDTGEDTQLYLLSNATGTGWDPPGIPELILEGFPLWLIYWGFADFQKNLKKVTNID |
| | TNYMLVAKTKFTQKPGTFYLVILNDTFVEGNSPYEKQPLPEDNIKWYPQVQYQLEA |
| | QNKLLQTGPFTPNIQGQLSDNISMFYKFYFKWGGSPPKAINVENPAHQIQYPIPRNE |
| | HETTSLQSPGEAPESILYSFDYRHGNYTTTALSRISQDWALKDTVSKITEPDRQQLLK |
| | QALECLQISEETQEKKEKEVQQLISNLRQQQQLYRERIISLLKDQ (SEQ ID NO: 45) |
| ORF1/1 | MPYYYRRRRYNYRRPRWYGRGWIRRPFRRRFRRKRRIQYPIPRNEHETTSLQSPGE |
| | APESILYSFDYRHGNYTTTALSRISQDWALKDTVSKITEPDRQQLLKQALECLQISEE |
| | TQEKKEKEVQQLISNLRQQQQLYRERIISLLKDQ (SEQ ID NO: 46) |
| ORF1/2 | MPYYYRRRRYNYRRPRWYGRGWIRRPFRRRFRRKRRSQIDSNCSNKPSNACKSRK |
| | KRRRKKKKKYSSSSATSDSSSSCTESE (SEQ ID NO: 47) |

TABLE 13
Exemplary Anellovirus nucleic acid sequence
(Gammatorquevirus)

| | |
|---|---|
| Name | TTMDV-MD1-073 |
| Genus/Clade | Gammatorquevirus |
| Accession Number | AB290918.1 |
| Full Sequence: | 3242 bp |

1 10 20 30 40 50
| | | | | |
AGGTGGAGACTCTTAAGCTATATAACCAAGTGGGGTGGCGAATGGCTGAG
TTTACCCCGCTAGACGGTGCAGGGACCGGATCGAGCGCAGCGAGGAGGTC
CCCGGCTGCCCGTGGGCGGGAGCCCGAGGTGAGTGAAACCACCGAGGTCT
AGGGGCAATTCGGGCTAGGGCAGTCTAGCGGAACGGGCAAGAAACTTAAA
AATATTTCTTTTACAGATGCAAAACCTATCAGCCAAAGACTTCTACAAAC
CATGCAGATACAACTGTGAAACTAAAAACCAAATGTGGATGTCTGGCATT
GCTGACTCCCATGACAGTTGGTGTGACTGTGATACTCCTTTTGCTCACCT
CCTGGCTAGTATTTTTCCTCCTGGTCACACAGATCGCACACGAACCATCC
AAGAAATACTTACCAGAGATTTTAGGAAAACATGCCTTTCTGGTGGGGCC
GACGCAACAAATTCTGGTATGGCCGAAACTATAGAAGAAAAAAGAGAAGA
TTTCCAAAAAGAAGAAAAAGAAGATTTTACAGAAGAACAAAATATAGAAG
ACCTGCTCGCCGCCGTCGCAGACGCAGAAGGAAGGTAAGAAGAAAAAAAA
AAACTCTTATAGTAAGACAATGGCAGCCAGACTCTATTGTACTCTGTAAA
ATTAAAGGGTATGACTCTATAATATGGGGAGCTGAAGGCACACAGTTTCA
ATGTTCTACACATGAAATGTATGAATATACAAGACAAAAGTACCCTGGGG
GAGGAGGATTTGGTGTACAACTTTACAGCTTAGAGTATTTGTATGACCAA
TGGAAACTTAGAAATAATATATGGACTAAAACAAATCAACTCAAAGATTT
GTGTAGATACTTAAAATGTGTTATGACCTTTTACAGACACCAACACATAG
ATTTTGTAATTGTATATGAAAGACAACCCCCATTTGAAATAGATAAACTA
ACATACATGAAATATCATCCATATATGTTATTACAAAGAAAGCATAAAAT
AATTTTACCTAGTCAAACAACTAATCCTAGAGGTAAATTAAAAAAAAAGA
AAACTATTAAACCTCCCAAACAAATGCTCAGCAAATGGTTTTTTCAACAA
CAATTTGCTAAATATGATCTACTACTTATTGCTGCAGCAGCATGTAGTTT
AAGATACCCTAGAATAGGCTGCTGCAATGAAAATAGAATGATAACCTTAT
ACTGTTTAAATACTAAATTTTATCAAGATACAGAATGGGGAACTACAAAA
CAGGCCCCCCACTACTTTAAACCATATGCAACAATTAATAAATCCATGAT
ATTTGTCTCTAACTATGGAGGTAAAAAAACAGAATATAACATAGGCCAAT
GGATAGAAACAGATATACCTGGAGAAGGTAATCTAGCAAGATACTACAGA
TCAATAAGTAAAGAAGGAGGTTACTTTTCACCTAAAATACTGCAAGCATA
TCAAACAAAAGTAAAGTCTGTAGACTACAAACCTTTACCAATTGTTTTAG
GTAGATATAACCCAGCAATAGATGATGGAAAAGGCAACAAAATTTACTTA
CAAACTATAATGAATGGCCATTGGGGCCTACCTCAAAAAACACCAGATTA
TATAATAGAAGAGGTCCCTCTTTGGCTAGGCTTCTGGGGATACTATAACT
ACTTAAAACAAACAAGAACTGAAGCTATATTTCCACTACACATGTTTGTA
GTGCAAAGCAAATACATTCAAACACAACAAACAGAAACACCTAACAATTT
TTGGGCATTTATAGACAACAGCTTTATACAGGGCAAAAACCCATGGGACT
CAGTTATTACTTACTCAGAACAAAAGCTATGGTTTCCTACAGTTGCATGG
CAACTAAAAACCATAAATGCTATTTGTGAAAGTGGACCATATGTACCTAA
ACTAGACAATCAAACATATAGTACCTGGGAACTAGCAACTCATTACTCAT
TTCACTTTAAATGGGGTGGTCCACAGATATCAGACCAACCAGTTGAAGAC
CCAGGAAACAAAAACAAATATGATGTGCCCGATACAATCAAAGAAGCATT
ACAAATTGTTAACCCAGCAAAAAACATTGCTGCCACGATGTTCCATGACT
GGGACTACAGACGGGGTTGCATTACATCAACAGCTATTAAAAGAATGCAA
CAAAACCTCCCAACTGATTCATCTCTCGAATCTGATTCAGACTCAGAACC
AGCACCCAAGAAAAAAAGACTACTACCAGTCCTCCACGACCCACAAAAGA
AAACGGAAAAGATCAACCAATGTCTCCTCTCTCTCTGCGAAGAAAGTACA
TGCCAGGAGCAGGAAACGGAGGAAAACATCCTCAAGCTCATCCAGCAGCA
GCAGCAGCAGCAGCAGAAACTCAAGCACAACCTCTTAGTACTAATCAAGG
ACTTAAAAGTGAAACAAAGATTATTACAACTACAAACGGGGGTACTAGAA
TAACCCTTACCAGATTTAAACCAGGATTTGAGCAAGAAACTGAAAAAGAG
TTAGCACAAGCATTTAACAGACCCCCTAGACTGTTCAAAGAAGATAAACC
CTTTTACCCCTGGCTACCCAGATTTACACCCCTTGTAAACTTTCACCTTA
ATTTTAAAGGCTAGGCCTACACTGCTCACTTAGTGGTGTATGTTTATTAA
AGTTTGCACCCCAGAAAAATTGTAAAATAAAAAAAAAAAAAAAAAATAAA
AAATTGCAAAAATTCGGCGCTCGCGCGCGCTGCGCGCGCGAGCGCCGTCA
CGCGCCGGCGCTCGCGCGCCGCGCGTATGTGCTAACACACCACGCACCTA
GATTGGGGTGCGCGCGTAGCGCGCGCACCCCAATGCGCCCCGCCCTCGTT
CCGACCCGCTTGCGCGGGTCGGACCACTTCGGGCTCGGGGGGGCGCGCCT
GCGGCGCTTATTTACTAAACAGACTCCGAGTCGCCATTGGGCCCCCCCTA
AGCTCCGCCCCCCTCATGAATATTCATAAAGGAAACCACAAAATTAGAAT
TGCCGACCACAAACTGCCATATGCTAATTAGTTCCCCTTTTACACAGTAA
AAAGGGGAAGTGGGGGGGCAGAGCCCCCCCACACCCCCCGCGGGGGGGGC
AGAGCCCCCCCCGCACCCCCCCTACGTCACAGGCCACGCCCCCGCCGCCA
TCTTGGGTGCGGCAGGGCGGGGACTAAAATGGCGGGACCCAATCATTTTA
TACTTTCACTTTCCAATTAAAACCCGCCACGTCACACAAAAG
(SEQ ID NO: 48)
Annotations:
Putative Domain Base range
TATA Box 21-25
Cap Site 42-49
Transcriptional Start Site 49
5′ UTR Conserved Domain 117-187
ORF2 283-588
ORF2/2 283-584; 1977-2388
ORF2/3 283-584; 2197-2614
ORF1 432-2453
ORF1/1 432-584; 1977-2453
ORF1/2 432-584; 2197-2388
Three open-reading frame region 2186-2385
Poly(A) Signal 2676-2681
GC-rich region 3054-3172

TABLE 14
Exemplary Anellovirus amino acid sequences (Gammatorquevirus)
TTMDV-MD1-073 (Gammatorquevirus)

| | |
|---|---|
| ORF2 | MWMSGIADSHDSWCDCDTPFAHLLASIFPPGHTDRTRTIQEILTRDFRKTCLSGGAD |
| | ATNSGMAETIEEKREDFQKEEKEDFTEEQNIEDLLAAVADAEGR (SEQ ID NO: 49) |
| ORF2/2 | MWMSGIADSHDSWCDCDTPFAHLLASIFPPGHTDRTRTIQEILTRDFRKTCLSGGAD |
| | ATNSGMAETIEEKREDFQKEEKEDFTEEQNIEDLLAAVADAEGRYQTNQLKTQETK |
| | TNMMCPIQSKKHYKLLTQQKTLLPRCSMTGTTDGVALHQQLLKECNKTSQLIHLSN |
| | LIQTQNQHPRKKDYYQSSTTHKRKRKRSTNVSSLSAKKVHARSRKRRKTSSSSSSSS |
| | SSSSRNSSTTS (SEQ ID NO: 50) |
| ORF2/3 | MWMSGIADSHDSWCDCDTPFAHLLASIFPPGHTDRTRTIQEILTRDFRKTCLSGGAD |
| | ATNSGMAETIEEKREDFQKEEKEDFTEEQNIEDLLAAVADAEGRTSTQEKKTTTSPP |
| | RPTKENGKDQPMSPLSLRRKYMPGAGNGGKHPQAHPAAAAAAAETQAQPLSTNQ |
| | GLKSETKIITTTNGGTRITLTRFKPGFEQETEKELAQAFNRPPRLFKEDKPFYPWLPRF |
| | TPLVNFHLNFKG (SEQ ID NO: 51) |
| ORF1 | MPFWWGRRNKFWYGRNYRRKKRRFPKRRKRRFYRRTKYRRPARRRRRRRRKVR |
| | RKKKTLIVRQWQPDSIVLCKIKGYDSIIWGAEGTQFQCSTHEMYEYTRQKYPGGGG |
| | FGVQLYSLEYLYDQWKLRNNIWTKTNQLKDLCRYLKCVMTFYRHQHIDFVIVYER |
| | QPPFEIDKLTYMKYHPYMLLQRKHKIILPSQTTNPRGKLKKKKTIKPPKQMLSKWFF |
| | QQQFAKYDLLLIAAAACSLRYPRIGCCNENRMITLYCLNTKFYQDTEWGTTKQAPH |
| | YFKPYATINKSMIFVSNYGGKKTEYNIGQWIETDIPGEGNLARYYRSISKEGGYFSPK |
| | ILQAYQTKVKSVDYKPLPIVLGRYNPAIDDGKGNKIYLQTIMNGHWGLPQKTPDYII |
| | EEVPLWLGFWGYYNYLKQTRTEAIFPLHMFVVQSKYIQTQQTETPNNFWAFIDNSFI |
| | QGKNPWDSVITYSEQKLWFPTVAWQLKTINAICESGPYVPKLDNQTYSTWELATH |
| | YSFHFKWGGPQISDQPVEDPGNKNKYDVPDTIKEALQIVNPAKNIAATMFHDWDY |
| | RRGCITSTAIKRMQQNLPTDSSLESDSDSEPAPKKKRLLPVLHDPQKKTEKINQCLLS |
| | LCEESTCQEQETEENILKLIQQQQQQQQKLKHNLLVLIKDLKVKQRLLQLQTGVLE |
| | (SEQ ID NO: 52) |
| ORF1/1 | MPFWWGRRNKFWYGRNYRRKKRRFPKRRKRRFYRRTKYRRPARRRRRRRRKISD |
| | QPVEDPGNKNKYDVPDTIKEALQIVNPAKNIAATMFHDWDYRRGCITSTAIKRMQQ |
| | NLPTDSSLESDSDSEPAPKKKRLLPVLHDPQKKTEKINQCLLSLCEESTCQEQETEEN |
| | ILKLIQQQQQQQQKLKHNLLVLIKDLKVKQRLLQLQTGVLE (SEQ ID NO: 53) |
| ORF1/2 | MPFWWGRRNKFWYGRNYRRKKRRFPKRRKRRFYRRTKYRRPARRRRRRRRKISD |
| | QPVEDPGNKNKYDVPDTIKEALQIVNPAKNIAATMFHDWDYRRGCITSTAIKRMQQ |
| | NLPTDSSLESDSDSEPAPKKKRLLPVLHDPQKKTEKINQCLLSLCEESTCQEQETEEN |
| | ILKLIQQQQQQQQKLKHNLLVLIKDLKVKQRLLQLQTGVLE (SEQ ID NO: 54) |

In some embodiments, a synthetic curon comprises a minimal Anellovirus genome, e.g., as identified according to the method described in Example 9. In some embodiments, a synthetic curon comprises an Anellovirus sequence, or a portion thereof, as described in Example 13.

In some embodiments, a synthetic curon comprises a genetic element comprising a consensus Anellovirus motif, e.g., as shown in Table 14-1. In some embodiments, a synthetic curon comprises a genetic element comprising a consensus Anellovirus ORF1 motif, e.g., as shown in Table 14-1. In some embodiments, a synthetic curon comprises a genetic element comprising a consensus Anellovirus ORF1/1 motif, e.g., as shown in Table 14-1. In some embodiments, a synthetic curon comprises a genetic element comprising a consensus Anellovirus ORF1/2 motif, e.g., as shown in Table 14-1. In some embodiments, a synthetic curon comprises a genetic element comprising a consensus Anellovirus ORF2/2 motif, e.g., as shown in Table 14-1. In some embodiments, a synthetic curon comprises a genetic element comprising a consensus Anellovirus ORF2/3 motif, e.g., as shown in Table 14-1. In some embodiments, a synthetic curon comprises a genetic element comprising a consensus Anellovirus ORF2t/3 motif, e.g., as shown in Table 14-1. In some embodiments, X, as shown in Table 14-1, indicates any amino acid. In some embodiments, Z, as shown in Table 14-1, indicates glutamic acid or glutamine. In some embodiments, B, as shown in Table 14-1, indicates aspartic acid or asparagine. In some embodiments, J, as shown in Table 14-1, indicates leucine or isoleucine.

TABLE 14-1
Consensus motifs in open reading frames (ORFs) of Anelloviruses

| | Open | | | |
|---|---|---|---|---|
| Consensus | Reading | | | SEQ ID |
| Threshold | Frame | Position | Motif | NO: |
| 50 | ORF1 | 79 | LIJRQWQPXXIRRCXIXGYXPLIXC | 55 |
| 50 | ORF1 | 111 | NYXXHXD | 56 |
| 50 | ORF1 | 135 | FSLXXLYDZ | 57 |
| 50 | ORF1 | 149 | NXWTXSNXDLDLCRYXGC | 58 |
| 50 | ORF1 | 194 | TXPSXHPGXMXLXKHK | 59 |
| 50 | ORF1 | 212 | IPSLXTRPXG | 60 |
| 50 | ORF1 | 228 | RIXPPXLFXDKWYFQXDL | 61 |
| 50 | ORF1 | 250 | LLXIXATA | 62 |
| 50 | ORF1 | 260 | LXXPFXSPXTD | 63 |
| 50 | ORF1 | 448 | YNPXXDKGXGNXIW | 64 |
| 50 | ORF1 | 519 | CPYTZPXL | 65 |
| 50 | ORF1 | 542 | XFGXGXMP | 66 |
| 50 | ORF1 | 569 | HQXEVXEX | 67 |
| 50 | ORF1 | 600 | KYXFXFXWGGNP | 68 |
| 50 | ORF1 | 653 | HSWDXRRG | 69 |
| 50 | ORF1 | 666 | AIKRXQQ | 70 |
| 50 | ORF1 | 750 | XQZQXXLR | 71 |
| 50 | ORF1/1 | 73 | PRXJQXXDP | 72 |
| 50 | ORF1/1 | 91 | HSWDXRRG | 73 |
| 50 | ORF1/1 | 105 | AIKRXQQ | 74 |
| 50 | ORF1/1 | 187 | QZQXXLR | 75 |
| 50 | ORF1/2 | 97 | KXKRRRR | 76 |
| 50 | ORF2/2 | 158 | PIXSLXXYKXXTR | 77 |
| 50 | ORF2/2 | 189 | LAXQLLKECXKN | 78 |
| 50 | ORF2/3 | 39 | HLNXLA | 79 |
| 50 | ORF2/3 | 272 | DRPPR | 80 |
| 50 | ORF2/3 | 281 | DXPFYPWXP | 81 |
| 50 | ORF2/3 | 300 | VXFKLXF | 82 |
| 50 | ORF2t/3 | 4 | WXPPVHBVXGIERXW | 83 |
| 50 | ORF2t/3 | 37 | AKRKLX | 84 |
| 50 | ORF2t/3 | 140 | PSSXDWXXEY | 85 |
| 50 | ORF2t/3 | 156 | DRPPR | 86 |
| 50 | ORF2t/3 | 167 | PFYPW | 87 |
| 50 | ORF2t/3 | 183 | NVXFKLXF | 88 |
| 50 | ORF1 | 84 | JXXXXWQPXXXXXCXIXGXXXJWQP | 89 |
| 50 | ORF1 | 149 | NXWXXXNXXXXLXRY | 90 |
| 50 | ORF1 | 448 | YNPXXDXG | 91 |

### Genetic Element

In some embodiments, the curon comprises a genetic element. In some embodiments, the genetic element has one or more of the following characteristics: is substantially non-integrating with a host cell's genome, an episomal nucleic acid, a single stranded DNA, is circular, is about 1 to 10 kb, exists within the nucleus of the cell, can be bound by endogenous proteins, and produces a microRNA that targets host genes. In one embodiment, the genetic element is a substantially non-integrating DNA. In some embodiments, the genetic element has at least about 70%, 75%, 80%, 8%, 90%, 95%, 96%, 97%, 98%, 99%, or 100% sequence identity to an Anellovirus sequence, e.g., as described herein (e.g., as described in any of Tables 1-14), or a fragment thereof. In embodiments, the genetic element comprises a sequence encoding an exogenous effector (e.g., a payload), e.g., a polypeptide effector (e.g., a protein) or nucleic acid effector (e.g., a non-coding RNA, e.g., a miRNA, siRNA, mRNA, lncRNA, RNA, DNA, an antisense RNA, gRNA).

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences.

In one embodiment, the invention includes a genetic element comprising a nucleic acid sequence (e.g., a DNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence.

Proteins, e.g., Substantially Non-Pathogenic Protein

In some embodiments, the genetic element comprises a sequence that encodes a protein, e.g., a substantially non-pathogenic protein. In embodiments, the substantially non-pathogenic protein is a major component of the proteinaceous exterior of the curon. Multiple substantially non-pathogenic protein molecules may self-assemble into an icosahedral formation that makes up the proteinaceous exterior. In embodiments, the protein is present in the proteinaceous exterior.

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more.

In some embodiments, the protein, e.g., substantially non-pathogenic protein and/or proteinaceous exterior protein comprises at least one hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

In some embodiments, the genetic element comprises a nucleotide sequence encoding a capsid protein or a fragment of a capsid protein or a sequence having at least about 60%, 65%, 70%, 75%, 80%, 85%, 90% 95%, 96%, 97%, 98%, 99%, or 100% nucleotide sequence identity to any one of the nucleotide sequences encoding a capsid protein described herein, e.g., as listed in any of Tables 1-16 or 19. In some embodiments, the genetic element comprises a nucleotide sequence encoding a capsid protein or a functional fragment of a capsid protein or a nucleotide sequence having at least about 60%, 70% 80%, 85%, 90% 95%, 96%, 97%, 98%, 99%, or 100% sequence identity to any one of the nucleotide sequences described herein, e.g., as listed in any of Tables 1-16 or 19. In some embodiments, the substantially non-pathogenic protein comprises a capsid protein or a functional fragment of a capsid protein that is encoded by a capsid nucleotide sequence or a sequence having at least about 60%, 65%, 70%, 75%, 80%, 85%, 90% 95%, 96%, 97%, 98%, 99%, or 100% nucleotide sequence identity to any one of the nucleotide sequences described herein, e.g., as listed in any of Tables 1, 3, 5, 7, 9, 11, 13, or 15.

TABLE 15
Examples of viral sequences that encode viral proteins, e.g.,
capsid proteins.

| Accession # | Accession # | | SEQ |
|---|---|---|---|
| (protein | (nucleotide | | ID |
| sequence) | sequence) | Sequence | NO: |
| AAD45640.1 | AF122917.1 | ATGCACTTTTCTAGGATATCCAGGAAGAAAAGGCTACTGCTACTGC | 92 |
| | | ACACAGTGCCAACTCCACAGAAAACTCTCAAACTTTTAAGAGGTAT | |
| | | GTGGAGTCCTCCCACTGACGATGAACGTGTCCGCGAGCGAAAATG | |
| | | GTTTCTCGCAACTGTCTATTCTCACTCTGCTTTCTGTGGCTGCAAT | |
| | | GATCCTGTCGGTCACCTCTGTCGCCTGGCTACTCTCTCTAACCGT | |
| | | CCGGAGAACCCGGGACCCTCCGGGGGACGTCGTGCTCCTTCGAT | |
| | | CGGGGTCCTACCCGCTCTCCCGGCTGCTACCGAGCAGCCAGGTG | |
| | | ATCGAGCACCATGGCCTATGGGTGGTGGAGGAGACGCCGCAGAA | |
| | | GGTGGAAGAGATGGAGGAGAAGGCCCAGGTGGAGACGCCCATGG | |
| | | AGGACCCGCAGACGCAGACCTGCTAGACGCCGTGGACGCCGCGG | |
| | | AACAGTAA | |
| AAD45641.1 | AF122917.2 | ATGGCCTATGGGTGGTGGAGGAGACGCCGCAGAAGGTGGAAGAG | 93 |
| | | ATGGAGGAGAAGGCCCAGGTGGAGACGCCCATGGAGGACCCGCA | |
| | | GACGCAGACCTGCTAGACGCCGTGGACGCCGCGGAACAGTAAGG | |
| | | AGACGGAGGCGCGGGAGGTGGAGGAGGCGCTATAGGAGGTGGA | |
| | | GGAGAAAGGGCAGACGCAGGAGAAAAAAGAAACTTATAATAAGAC | |
| | | AATGGCAGCCAAACTATACCAGAAAGTGCAACATAGTAGGCTACAT | |
| | | GCCAGTAATCATGTGTGGAGAAAACACTCTAATAAGAAACTATGCC | |
| | | ACACACGCAGACGACTGCTACTGGCCGGGACCCTTTGGGGGCGG | |
| | | CATGGCCACCCAGAAATTCACACCCAGAATCCTGTACGATGACTA | |
| | | CAAGAGGTTTATGAACTACTGGACCTCCTCAAACGAGGACCTAGA | |
| | | CCTCTGTAGATACAGGGGAGTCACCCTGTACTTTTTCAGACACCCA | |
| | | GATGTAGACTTTATCATCTTAATAAACACCACACCTCCATTCGTAGA | |
| | | TACAGAGATCACAGGACCCAGCATACATCCGGGCATGATGGCCCT | |
| | | GAACAAGAGAGCCAGGTTCATCCCCAGCCTAAAGACTAGACCTGG | |
| | | CAGAAGACACATAGTAAAGATTAGAGTGGGGGCCCCCAAACTGTA | |
| | | CGAGGACAAGTGGTACCCCCAGTCAGAACTCTGTGACGTGCCCCT | |
| | | GCTAACCGTCTACGCGACCGCAGCGGATATGCAATATCCGTTCGG | |
| | | CTCACCACTAACTGACACTCCTGTTGTAACCTTCCAAGTGTTGCGC | |
| | | AGCATGTACAACGACGCCCTCAGCACACTTCCCTCTAACTTTGAAA | |
| | | ACGCAAGCAGTCCAGGCCAAAAACTTTACAAAGAAATATCTACATA | |
| | | TTTACCATACTACAACACCACAGAAACAATAGCACAACTAAAGAGA | |
| | | TATGTAGAAAATACAGAAAAAAATGGCACAACGCCAAACCCGTGG | |
| | | CAATCAAAATATGTAAACACTACTGCCTTCACCACTGCACTAAATGT | |
| | | TACAACTGAAAAACCATACACCACCTTCTCAGACAGCTGGTACAGG | |
| | | GGCACAGTATACAAAGAAACAATCACTGAAGTGCCACTTGCCGCA | |
| | | GCAAAACTCTATCAAAACCAAACAAAAAAGCTGCTGTCTACAACAT | |
| | | TTACAGGAGGGTCCGAGTACCTAGAATACCATGGAGGCCTGTACA | |
| | | GCTCCATATGGCTATCAGCAGGCCGATCCTACTTTGAAACAAAGG | |
| | | GAGCATACACAGACATCTGCTACAACCCCTACACAGACAGAGGAG | |
| | | AGGGCAACATGGTGTGGATAGACTGGCTATCAAAAACAGACTCCA | |
| | | GATATGACAAAACCCGCAGCAAATGCCTTATAGAAAAGCTACCCCT | |
| | | ATGGGCAGCAGTATACGGGTACCCAGAATACTGTGCCAAGAGCAC | |
| | | CGGAGACTCAAACATAGACATGAACGCCAGAGTAGTAATAAGGTG | |
| | | CCCCTACACCGTCCCCCAGATGATAGACACCAGCGACGAACTAAG | |
| | | GGGCTTCATAGTATACAGCTTTAACTTTGGCAGGGGCAAAATGCC | |
| | | CGGAGGCAGCAGCGAGGTACCCATAAGAATGAGAGCCAAGTGGT | |
| | | ACCCCTGCCTGTTTCACCAAAAAGAAGTTCTAGAAGCCTTGGGACA | |
| | | GTCGGGCCCCTTCGCCTACCACTGCGACCAAAAAAAAGCAGTGCT | |
| | | AGGTCTAAAATACAGATTTCACTGGATATGGGGCGGAAGCCCCGT | |
| | | GTTTCCACAGGTTGTTAGAAACCCCTGCAAAGACACACACGGTTC | |
| | | CTCGGGCCCTAGAAAGCCTCGCTCAATACAAATCATTGACCCGAA | |
| | | GTACAACACACCAGAGCTCACAATCCACGCGTGGGATTTCAGACG | |
| | | TGGCTTCTTTGGCTCAAAAGCTATTAAAAGAATGCAACAACAACCA | |
| | | ACAGATGCTGAACTTCTTCCACCAGGCCGCAAGAGGAGCAGGCGA | |
| | | GACACAGAAGCCCTCCAAAGCAGCCAAGAAAAGCAAAAAGAAAGC | |
| | | TTACTTTTCAAACACCTCCAGCTCCAGCGACGAATACCCCCATGGG | |
| | | AAAGCTCGCAGGCCTCGCAGACAGAGGCAGAGAGCGAAAAAGAG | |
| | | CAAGAGGGCAGTCTCTCCCAGCAGCTCCGAGAGCAGCTTTACCAG | |
| | | CAAAAGCTCCTCGGCAAGCAGCTCAGGGAAATGTTCCTACAACTC | |
| | | CACAAAATCCAACAAAATCAACACGTCAACCCTACCTTATTGCCAA | |
| | | GGGATCAGGCTTTAATCTGCTGGTCTCAGATTCAGTAA | |
| AAD45642.1 | AF122917.1 | ATGTTTGGAGACCCTAAACCATACAAACCCTCCAGCAACGACTGG | 94 |
| | | AAAGAGGAGTACGAGGCCGCTAAGTATTGGGACAGGCCCCCCAG | |
| | | ATCTAACCTTAGAGATAACCCCTTCTATCCCTGGGCCCCCCCAAGC | |
| | | AATCCCTACAAAGTAAACTTTAAACTAGGCTTCCAATAA | |
| AAD45646.1 | AF122919.1 | ATGCACTTTTCTAGGATATCCAGAAAGAAAAGGCTACTGCTACTGC | 95 |
| | | AAACAGAGCCAGCTCCACAGAAGACTCTCAAACTTTTAAAAGGTAT | |
| | | GTGGAGTCCTCCCACTGACGATGAACGTGTCCGCGAGCGAAAATG | |
| | | GTTCCTCGCCACTGTTTATTCTCACTCTGCTTTCTGTGGCTGCAAT | |
| | | GATCCTGTCGGCCACCTCTGTCGCTTGGCTACTCTATCTAACCGTC | |
| | | CGGAGAACCCGGGACCCTCCGGGGGACGTCGTGCTCCTTCGATC | |
| | | GGGATCCTACCCGCTCTCCCGGCTGCTACCGAGCAGCCCGGTGA | |
| | | TCGAGCACCATGGCCTATGGGTGGTGGAGGAGACGCCGCAGAAG | |
| | | GTGGAAGAGATGGAGGAGAAGGCCCAGGTGGAGACGCCCATGGA | |
| | | GGACCCGCAGACGCAGACCTGCTAGACGCCGTGGACGCCGCAGA | |
| | | ACAGTAA | |
| AAD45647.1 | AF122919_2 | ATGGCCTATGGGTGGTGGAGGAGACGCCGCAGAAGGTGGAAGAG | 96 |
| | | ATGGAGGAGAAGGCCCAGGTGGAGACGCCCATGGAGGACCCGCA | |
| | | GACGCAGACCTGCTAGACGCCGTGGACGCCGCAGAACAGTAAGG | |
| | | AGACGGAGGCGCGGGAGGTGGAGGAGGCGCTATAGGAGGTGGA | |
| | | GGAGAAAGGGCAGACGCGGGAGAAAAAAGAAACTTATAATAAAAC | |
| | | AATGGCAGCCAAACTATACCAGAGAGTGCAACATAGTAGGCTACA | |
| | | TGCCAGTAATCATGTGTGGAGAGAACACTCTAATAAGAAACTATGC | |
| | | CACACACGCAGACGACTGCTACTGGCCGGGACCCTTTGGGGGCG | |
| | | GCATGGCCACCCAGAAATTCACACTCAGAATCCTGTACGATGACTA | |
| | | CAAGAGGTTTATGAACTACTGGACCTCCTCAAACGAGGACCTAGA | |
| | | CCTCTGTAGATACAGGGGAGTCACCCTGTACTTTTTCAGAAACCCA | |
| | | GATGTAGACTTTATCATCCTCATAAACACCACACCTCCGTTCGTAG | |
| | | ATACAGAGATCACAGGACCCAGCATACATCCGGGCATGATGGCCC | |
| | | TCAACAAAAGAGCCAGGTTCATCCCCAGCCTAAAAACTAGACCTG | |
| | | GCAGAAGACACATAGTAAAGATTAAAGTGGGGGCCCCCAAACTGT | |
| | | ACGAGGACAAGTGGTACCCCCAGTCAGAACTCTGTGACATGCCCC | |
| | | TACTAACCGTCTACGCCACCGCAGCGGATATGCAATATCCGTTCG | |
| | | GCTCACCACTAACTGACACTCCTGTTGTAACCTTCCAAGTGTTGCG | |
| | | CAGCATGTACAACGACGCCCTTAGCATACTTCCCTCTAACTTTCAA | |
| | | AGCCCAGACAGTCCAGGCCAAAAACTTTACGAACAAATATCTAAGT | |
| | | ATTTACCATACTACAACACCACAGAAACAATGGCACAACTAAAGAG | |
| | | ATATATAGAAAATACAGAAAAAAATACCACATCGCCAAACCCATGG | |
| | | CAAACAAAATATGTAAACACTACTGCCTTCACCACTCCACAAACTG | |
| | | TTACAACTCAACAGCCATACACCAGCTTCTCAGACAGCTGGTACAG | |
| | | GGGCACAGTATACACAAACGAAATCACTAAGGTGCCACTTGCCGC | |
| | | AGCAAAAGTGTATGAAACTCAAACAAAAAACCTGCTGTCTACAACA | |
| | | TTTACAGGAGGGTCAGAGTACCTAGAATACCATGGAGGCCTGTAC | |
| | | AGCTCCATATGGCTATCAGCAGGCCGATCCTACTTTGAAACAAAG | |
| | | GGAGCATACACAGACATCTGCTACAACCCCTACACAGACAGAGGA | |
| | | GAGGGCAACATGGTGTGGATAGACTGGCTATCAAAAACAGACTCC | |
| | | AGATATGACAAAACCCGCAGCAAATGCCTTATAGAAAAGCTACCCC | |
| | | TATGGGCAGCAGTATACGGGTACGCAGAATACTGTGCCAAGAGCA | |
| | | CCGGAGACTCAAACATAGACATGAACGCCAGAGTAGTAATTAGGT | |
| | | GCCCCTACACCACCCCCCAGATGATAGACACCAGCGACGAACTAA | |
| | | GGGGCTTCATAGTATACAGCTTTAACTTTGGCAGGGGCAAAATGC | |
| | | CCGGAGGCAGCAGCGAGGTACCCATTAGAATGAGAGCCAAGTGG | |
| | | TACCCCTGCCTACTTCACCAAAAAGGAGTTCTAGAAGCCTTAGGAC | |
| | | AGTCAGGCCCCTTCGCCTACCACCGCGACCAAAAAAAAGCAGTGC | |
| | | TAGGTCTAAAATACAGATTTCACTGGATATGGGGCGGAAACCCCG | |
| | | TGTTTCCACAGGTTGTTAGAAACCCCTGCAAAGACACACACGGTTC | |
| | | CTCGGGCCCTAGAAAGCCTCGCTCAATACAAATCATTGACCCGAA | |
| | | GTACAACACACCAGAGCTCACAATCCACGCGTGGGATTTCAGACG | |
| | | TGGCTTCTTTGGCCCAAAAGCTATTAAGAGAATGCAACAACAACCA | |
| | | ACAGATGCTGAACTTCTTCCACCAGGCCGCAAGAGGAGCAGGCGA | |
| | | GACACCGAAGCCCTCCAAAGCAGCCAAGAAAAGCAGAAAGAAAGC | |
| | | TTACTTTTCAAACAGCTCCAGCTCCGGCGACGAGTACCCCCGTGG | |
| | | GAAAGCTCGCAGGCCTCGCAGACAGAGGCAGAGAGCGAAAAAGA | |
| | | GCAAGAGGACAGTCTCTCCCAGCAGCTCCGAGAGCAGCTTCACCA | |
| | | GCAAAAGCTCCTCGGCAAGCAGCTCAGGGAAATGTTCCTACAACT | |
| | | CCACAAAATCCAACAAAATCAACACGTCAACCCTACCCTATTGCCA | |
| | | AAAGATCAGGCTTTAATATGCTGGTCTCAGATTCAGTAA | |
| AAD45648.1 | AF122919_3 | ATGTTCGGAGACCCTAAACCATACAAACCCTCCAGCAACGACTGG | 97 |
| | | AAAGAGGAGTACGAGGCCGCTAAATATTGGGACAGGCCCCCCAG | |
| | | ATTTGACCTTAGAGATAAGCCCTTCTATCCCTGGGCCCCCCCAAG | |
| | | CAATCCCTACAAAGTAAACTTTAAACTAGGCTTTCAATAA | |
| AAG16247.1 | AF298585_1 | ATGGCTGAGTTTTCCACGCCCGTCCGCAGCGGTGAAGCCACGGA | 98 |
| | | GGGACCTCAGCGCGTCCCGAGGGCGGGTGCCGAAGGTGAGTTTA | |
| | | CACACCGCAGTCAAGGGGCAATTCGGGCTCGGGACTGGCCGGGC | |
| | | TATGGGCAAGGCTCTTAA | |
| AAG16248.1 | AF298585_2 | ATGTTTCTCGGTAAACTTTACAGAAAGAAAAGGAAAGTGCTTCTGC | 99 |
| | | AGACTGTGCCAGACCCACAGAAGGCTAGGCGGCTTCTGATTATGT | |
| | | GGCAGCCCCCCGTGCACAAAGTACCCGGGATCGAGAGAAACTGG | |
| | | TACGAGAGTTGCTTTCGATCCCATGCTGCTGTGTGTGGCTGTGGC | |
| | | GACTTTGTTGGCCATCTTAATCATCTGGCAGCTACTCTGGGTCGCC | |
| | | CTCCGCGTTCTCGGCACCCCGGGGGCCCCGGCACTCCGCAGATA | |
| | | AGAAACCTGCCAGCGCTCCCGGCACCCCAGGGTGAGCCCGGTGA | |
| | | CAGAGCGCCATGGCCTACGGATGGTGGGGCCGCCGGCGCCGCT | |
| | | GGAGAAGATGGAGGACGCGGCGCAGACCGTGGAGAACCAGGAG | |
| | | ACGTAGAAGACGACGCGCTCCTCGCCGCTTTCGACCTCGTCGAAG | |
| | | AGTAA | |
| AAG16249.1 | AF298585_3 | ATGGCCTACGGATGGTGGGGCCGCCGGCGCCGCTGGAGAAGATG | 100 |
| | | GAGGACGCGGCGCAGACCGTGGAGAACCAGGAGACGTAGAAGAC | |
| | | GACGCGCTCCTCGCCGCTTTCGACCTCGTCGAAGAGTAAGGAGG | |
| | | CGCAGGGGGCGGTGGCGCAGACGGTATAGAAAATGGAGGAGACG | |
| | | CAGGGGCAGACGGACGCACAGAAAAAAGATAATCATAAAACAGTG | |
| | | GCAGCCGAACTTTATAAGACGCTGCTACATAATAGGCTACCTGCCT | |
| | | CTCATATTCTGTGGCGAGAACACCACCGCCAATAACTTTGCCACCC | |
| | | ACTCGGACGACATGATAGCCAAAGGACCGTGGGGGGGGGGCATG | |
| | | ACTACCACTAAGTTCACTTTGAGAATCCTGTACGACGAGTTTACCA | |
| | | GGTTTATGAACTTCTGGACTGTCAGTAACGAAGACCTAGACCTGTG | |
| | | TAGATACGTGAGCTGCAAACTGATATTCTTTAAGCACCCCACGGTA | |
| | | GACTTTATAGTCAGGATAAACACAGAGCCTCCGTTCCTAGACACTA | |
| | | ACCTGACCGCGGCACAGATTCACCCGGGCATCATGATGCTAAGCA | |
| | | AAAAACACATACTCATACCCTCTCTAAAGACCAGGCCTAGCAGAAA | |
| | | ACACAGGGTGGTCGTCAGGGTGGGCCCACCTAGACTGTTTCAAGA | |
| | | CAAGTGGTACCCCCAGTCAGACCTGTGTGACACAGTTCTGCTTTC | |
| | | CGTGTTTGCAACGGCCTGTGACTTGCAATATCCGTTCGGCTCACC | |
| | | ACTAACTGACAACCCTTGCGTCAACTTCCAGATTCTGGGGCACCA | |
| | | GTACAAAAACCACCTTAGTATTAGCTCCACAAACGATACCACTAAC | |
| | | AAACAACACTATGACAACACTTTATTTAACAAAATAGTATTATATAA | |
| | | CACTTTTCAAACAATAGCTCAGCTCAAAGAAACAGGACAACTCACA | |
| | | AACTTATGGAACGAAGTACAAAACACAACAGCACTGTCACCAAAAG | |
| | | GCACAAATGCAACTATAAGCAAAGACACCTGGTACAAAGGAAACA | |
| | | CATACAAAGACAAGATTAAAGAGTTAGCAGAAAAAACTCGAAGTAG | |
| | | ATTTGCAGCTGCAACAAAAGCAGCCCTGCCAAACTACCCTACAATC | |
| | | ATGTCCACAGACCTGTATGAGTACCACTCAGGCATATACTCCAGCA | |
| | | TATTCCTAGCAGCAGGCAGGAGCTACTTTGAGACCCCGGGGGCCT | |
| | | ACACAGACGTCATATACAACCCTTTTACAGACAAAGGCACAGGAAA | |
| | | CATGGTCTGGATAGACTACCTCACAAAACCAGACTCCATATACACA | |
| | | AAGAACAAAAGCAAATGCGAGATATTTGACGTACCCCTGTGGGCC | |
| | | ACCTTCACAGGATACTCAGAATTCTGTTCAAAAGTTACAGGAGACA | |
| | | CCGCCATTCACCTAACTGCCAGAGTAGTAGTCAGATGCCCCTACA | |
| | | CCGAGCCCATGCTAATAGACCACTCAGACCCCAACAGGGGCTTTG | |
| | | TACCATACTCCTTTAACTTTGGAGAGGGCAAGATGCCCGGAGGCT | |
| | | CCTCAAAAGTACCCATAAGAATGAGAGCCAAGTGGTACGTGAACA | |
| | | TGTTTCACCAGCAAGAATTCATGGAGGCCATAGTTGAGAGCGGAC | |
| | | CGCTTGCTTACAAGGGCGACATAAAATCAGCGGTACTCACCATGA | |
| | | AATACAGATTCCACTGGAAATGGGGCGGAAACCCTATATCCAAACA | |
| | | GGTCGTCCGGAATCCCTGCTCCACCTCCAGCACCTCCGCGGGCC | |
| | | ATCGAGGACCTCGCAGCATACAAGTCGTTGACCCGAAGCACGTTA | |
| | | CCCCGGAAGTCACCTGGCACTCGTGGGACATCAAGCGAGGTCTCT | |
| | | TTGGCAAAGCAGGTATTAAGAGAATGCAACAAGAATCAGATGCTCT | |
| | | TTACATTCCTACAGGACCACTCAAGAGGCCACGGAGGGACACCAA | |
| | | CGCCCAAGACCCAGAAGAGCAAAACGAAAGCTCAGGTTTCAGAGT | |
| | | CCAGCAGCGACTCCCCTGGGTCCACTCCAGCCAAGAAACGCAAA | |
| | | GCTCCCAAGAGGAGATGCAAGCGGAGGGGACGGTACAAGAACAA | |
| | | CTCCTCCTCCAGCTCCGAGAGCAGCGAGTACTCCGGTTCCAGCTC | |
| | | CAACAGCTCGCCAGCCAAGTCCTCAAAGTGCAAGCAGGGCAAGG | |
| | | CCTACACCCCCTATTATCTTCCCAAGCGTAA | |
| AAG16250.1 | AF298585_4 | ATGTTTGAGCCCCAGGGTCCCAAACCCATACAGGGCTACAACGAT | 101 |
| | | TGGTTAGAAGAGTACACCTGCTGTAAATTCTGGGACAGGCCTCCC | |
| | | AGAAAGCTACACACAGATACACCCTTTTACCCCTGGGCACCAAAAC | |
| | | CCCCAGACCAAGTGAGAGTCTCCTTTAAACTTAACTTCCAATAA | |
| AAL37158.1 | AF315076_2 | ATGTTTCTTGGCAGGGCCTGGAGAAAGAAAAGGCAAGTGCCACTG | 102 |
| | | CCGACACTGCCAGTGGTGCCGCTTCCACAACCTTCACCTATGAGC | |
| | | AGCCAGTGGAGACCCCCGGTTCACAATGTCCAGGGGCTGGAGCG | |
| | | CAATTGGTGGGAGTGCTTCTTCCGTTCTCATGCTTGTTTTTGTGGC | |
| | | TGTGGTGATGCTATTACTCATATTAATCATCTGGCGACTCGTTTTG | |
| | | GACGTCCTCCTACTACCTCAACTCCCCGAGGACCGCAGGCACCTC | |
| | | CAGTGACTCCGTACCCGGCCCTGCCGGCCCCAGAGCCTAGCCCT | |
| | | GAGCCATGGCGTGGCGCCGGTGGCGATGGCGGCCGTGGTGGAG | |
| | | ACGCCGGAGGCGCCGCCGGTGGAGAAGGAGACGGAGGAGACCC | |
| | | AGACGACGCCGCCCTTATCGACGCCGTCGACCTCGCAGAGTAA | |
| AAL37157.1 | AF315076_1 | ATGGCGTGGCGCCGGTGGCGATGGCGGCCGTGGTGGAGACGCC | 103 |
| | | GGAGGCGCCGCCGGTGGAGAAGGAGACGGAGGAGACCCAGACG | |
| | | ACGCCGCCCTTATCGACGCCGTCGACCTCGCAGAGTAAGGAGGC | |
| | | GCAGGGGGCGGTGGAGGCGCGCGTACAGACGTTGGGGGCGACG | |
| | | CAGACGCAGACGCAGGCACAAAAAGAAACTTGTACTGACTCAGTG | |
| | | GCAACCAGCAGTAGTTAAGAGGTGCCTAATAGTGGGCTTTGACCC | |
| | | CCTTATAATATGTGGCATTAACAGAACAATATTTAACTACACTACAC | |
| | | ACTCTGAAGACTTTACTTTTAACAACGACAGCTTTGGAGGGGGGCT | |
| | | CTGTACCGCTCAGTACACACTAAGAATCCTTTTCCAAGAAAAGCTG | |
| | | GCCCAGCACAACTTCTGGTCAGCTAGCAACGAAGACCTAGACCTT | |
| | | GCCAGGTACCTAGGAGCCACAATAGTACTTTACAGACACCCTACA | |
| | | GTAGACTTCTTAGTTAGAATTCGCACCAGTCCTCCCTTTGAGGACA | |
| | | CAGACATGACAGCCATGACACTACATCCAGGCATGATGATGCTAG | |
| | | CTAAAAAGACAATTAAAATTCCCAGTCTTAAAACAAGACCGTCCAG | |
| | | AAAACACGTAGTAAGGATTAGAGTAGGGGCCCCTAAACTATTTGAA | |
| | | GACAAGTGGTACCCCCAGAACGAGCTATGTGATGTAACTCTGCTA | |
| | | ACCATACAGGCAACCACAGCTGATTTCCAATATCCGTTCGGCTCAC | |
| | | CACTAACGAACTCCCCCTGTTGCAACTTCCAGGTTCTTAACAGTAA | |
| | | CTATGACAATGCACATTCCATACTTAACTTGTCAAACGAACCAACA | |
| | | AACAAATGGCACACCTATAGAAATAACTGCTATAAATTTCTACTAGA | |
| | | ACAGTACAGCTACTACAACACTAAACAAGTAGTAGCACAACTTAAA | |
| | | TATAAATGGAACCCTAATCAAAACCCTACTATGCCAAATACAAGCA | |
| | | ATGCATCACTTTCTAAAAAACCTGATGACCTTACTAAAACCAAAACA | |
| | | ACAAACGAGTATCCACATTGGGACACCCTATATGGTGGTTTAGCAT | |
| | | ATGGACACAGCACTGTAACACCTGGCACTACCTCATCACCAACAG | |
| | | ACCTAAAAACACAAATGCTTACAGGCAACGAATTTTATACAACAGC | |
| | | AGGCAAAAAGTTAATAGATACATTTCACCCAATTCCTTACTATGAAA | |
| | | ACGGATCTTCTAAAGCCAACACCAACATATTTGACTACTACACAGG | |
| | | CATGTACAGTAGTATTTTCCTGTCTTCAGGCAGATCAAACCCAGAA | |
| | | GTAAAGGGCAGCTACACAGACATCTCTTACAACCCTCTGACAGAC | |
| | | AAGGGAGTAGGTAACATGATTTGGATAGACTGGCTCACTAAAGGA | |
| | | GACACAGTATACGACCCCAAAAAAAGCAAGTGCCTACTCTCAGACT | |
| | | TTCCATTGTGGTCACTTTGTTATGGATACCCAGACTACTGCAGAAA | |
| | | ACAAACCGGAGACTCAGGTATTTACTATGACTACAGAGTACTTATA | |
| | | AGATGTCCATACACATACCCTCAATTAATAAAACACAACGACAAAT | |
| | | ACTTTGGCTTCGTAGTGTACAGCGAAAACTTTGGACTGGGGCGAC | |
| | | TACCAGGAGGCAACCCTAACCCCCCAACTAGAATGAGACTGCACT | |
| | | GGTACCCTAATATGTTCCACCAAACAGAAGTACTAGAGTGCATAGC | |
| | | TCAAAGCGGACCGTTTGCTTATCATGGAGACGAGAGAAAAGCTGT | |
| | | TCTGACTGCCAAATACAAGTTCAGATGGAAGTGGGGAGGCAATCC | |
| | | TGTGTTTCAACAGGTTCTCCGAGACCCCTGCACCGGAGGTGCCGT | |
| | | GGCGCCCCACACCAGTCGACACCCTCGTGCAATACAAGTCCATGA | |
| | | CCCGAAGTATCAGGCCCCGGAGTACCTCTTCCACAAATGGGACTT | |
| | | CAGAAGGGGACTGTTTAGCACTAAAGGTATTAAGAGAGTGTCAGA | |
| | | ACAACCAGTACATGATGAGTATTTTACAGGGAGCAGCAAGAGACC | |
| | | CAAGAAAGACACCAACCCAAGCCCCCAAGGAGAAGAGCAAAAAGA | |
| | | AGGCTCGCGTTTCAGAGTCCCAGAGCTCAGACCCTGGCTCCCCTC | |
| | | CAGCCAGGAAACGCAGAGCCAAAGCGAGCAAGAAGAAACAGCCC | |
| | | CGAAAACGGTCCAAGAGCAGCTACAAGAACAACTCCAGCAGCAGC | |
| | | AGCTCATGGGAATCCAGCTCAGAAACGTCTGTCTCCAGCTCGCAA | |
| | | GAGTCCAAGCGGGGCACAGTCTCCACCCCGTTTTCCAATGCCATG | |
| | | CATAA | |
| AAL37159.1 | AF315076_3 | ATGACCCGAAGTATCAGGCCCCGGAGTACCTCTTCCACAAATGGG | 104 |
| | | ACTTCAGAAGGGGACTGTTTAGCACTAAAGGTATTAAGAGAGTGTC | |
| | | AGAACAACCAGTACATGATGAGTATTTTACAGGGAGCAGCAAGAG | |
| | | ACCCAAGAAAGACACCAACCCAAGCCCCCAAGGAGAAGAGCAAAA | |
| | | AGAAGGCTCGCGTTTCAGAGTCCCAGAGCTCAGACCCTGGCTCCC | |
| | | CTCCAGCCAGGAAACGCAGAGCCAAAGCGAGCAAGAAGAAACAG | |
| | | CCCCGAAAACGGTCCAAGAGCAGCTACAAGAACAACTCCAGCAGC | |
| | | AGCAGCTCATGGGAATCCAGCTCAGAAACGTCTGTCTCCAGCTCG | |
| | | CAAGAGTCCAAGCGGGGCACAGTCTCCACCCCGTTTTCCAATGCC | |
| | | ATGCATAAACAAAGTTTTTATTTTCCCTGA | |
| AAL37160.1 | AF315077_1 | ATGTTTCTCGGTAAACTTTACAGAAAGAAAAGGAAACTGCTACTGC | 105 |
| | | AAGCTGTGCGAGCTCCACAGGCGCCATCTTCCATGAGCTCCTCCT | |
| | | GGCGAGTGCCCCGCGGCGATGTCTCCGCCCGCGAGCTATGTTGG | |
| | | TACCGCTCAGTTCGAGAGAGCCACGATGCTTTTTGTGGCTGTCGT | |
| | | GATCCTGTTTTTCATCTTTCTCGTCTGGCTGCACGTTCTAACCATCA | |
| | | GGGACCTCCGACGCCCCCCACGGACGAGCGCCCGTCGGCGTCTA | |
| | | CCCCAGTGAGGCGCCTGCTGCCGCTGCCCTCCTACCCCGGCGAG | |
| | | GGTCCCCAGGCTAGATGGCCTGGTGGAGATGGAGAAGGCGCTGG | |
| | | TGACGCCCGCGGAGGCGCTGGAGATGGCGGCGCCCGCGCAGGC | |
| | | GAAGAAGAGTACCGGCCCGAAGACCTCGACGAGCTGTTCGGCGC | |
| | | TACCGAACAAGAACAGTAA | |
| AAL37161.1 | AF315077_2 | ATGCCAGTTATCTGGGCGGGCATGGGCACGGGGGGCCAAAACTA | 106 |
| | | CGCCGTCCGCTCAGATGACTTTGTAGTAGACAAGGGCTTCGGGGG | |
| | | CTCCTTCGCTACAGAGACTTTCTCCTTGAGAGTACTGTATGACCAG | |
| | | CACCAGAGGGGCTTTAACCGGTGGTCCCACACCAACGAGGACCTA | |
| | | GACCTTGCCCGTTACAGGGGATGCAAATGGACCTTTTACAGACAC | |
| | | CCAGACACTGACTTTATAGTGTACTTCACTAACAATCCCCCCATGA | |
| | | AAACTAACCAGTACACTGCCCCTCTCACCACTCCTGGAATGCTCAT | |
| | | GAGAAGCAAATATAAGATACTAATACCTAGTTTTAAAACAAAACCCA | |
| | | AGGGAAAAAAGACAATAAGCTTCAGAGCCAGACCCCCAAAACTAT | |
| | | TCCAAGACAAGTGGTACACTCAACAAGACCTCTGCCCTGTGCCCC | |
| | | TCATCCAACTGAACTTAACCGCAGCTGATTTCACACATCCGTTCGG | |
| | | CTTACCACTAACTGACTCTCCTTGCGTAAGGTTCCAAGTCCTCGGA | |
| | | GACTTGTACAATAACTGTCTCAATATAGACCTTCCGCAATTTGATGA | |
| | | CAAGGGTACAATTTCAGACGCATCCTCTTACAGTAGAGATAATAAG | |
| | | CAGCAGTTAGAAGAATTATATAAAACTCTATTTGTTAAAAAGGGCTG | |
| | | CGGACACTACTGGCAAACATTCATGACCAATAGCATGGTAAAAGCA | |
| | | CACATAGATGCTGCACAGGCACAAAACCATCAACAAGACACCTCA | |
| | | GGCCCTCAAAGTGCAAAAGATCCATTTCCAACAAAACCTGACAGAA | |
| | | ACCAATTTGAACAATGGAAAAACAAATTCACAGACCCCAGAGACAG | |
| | | CAACTTTCTCTTTGCCACTTATCACCCAGAAAACATTACACAGACTA | |
| | | TCAAAACAATGAGAGACAATAACTTTGCTCTAGAAACTGGAAAGAA | |
| | | TGACCTTTATGGTGATTATCAGGCCCAGTATACTAGAAACACTCAC | |
| | | CTTCTAGACTACTACCTGGGCTTCTACAGCCCCATATTCTTGTCCA | |
| | | GTGGCAGATCCAATACTGAATTCTTTACTGCCTACAGAGACATAAT | |
| | | ATACAATCCACTACTAGACAAAGGCACAGGTAATATGATTTGGTTC | |
| | | CAATACCACACAAAGACTGACAACATATTTAAAAAACCAGAGTGCC | |
| | | ACTGGGAAATACTAGACATGCCCCTGTGGGCCCTCTGCAACGGCT | |
| | | ACAAAGAGTACCTAGAGAGCCAAATAAAATATGGTGATATCTTAGT | |
| | | AGAAGGCAAAGTCCTCATAAGATGCCCATACACCAAACCTCCCCTA | |
| | | GCAGACCCCAACAACAGTCTAGCAGGATATGTAGTCTACAACACA | |
| | | AACTTTGGACAAGGCAAGTGGATCGACGGCAAGGGCTACATACCC | |
| | | CTAAGACACAGGAGCAAGTGGTATGTCATGCTCATGTACCAGACG | |
| | | GACGTACTCCATGACCTAGTGACTTGTGGACCCTGGCAATACAGA | |
| | | GACGATAATAAGAACTCTCAACTGATAGCCAAGTATAGATTTACTTT | |
| | | CTACTGGGGAGGTAACATGGTACATTCTCAGGTCATCAGGAACCC | |
| | | GTGCAAAGACACCCAAGTATCCGGCCCCCGTCGACAGCCTAGAGA | |
| | | GATACAAGTCGTTGACCCGCAACTCATCACCCCGCCGTGGGTCCT | |
| | | CCACTCGTTCGACCAGAGACGAGGAATGTTTACTGAGACAGCTAT | |
| | | CAGACGTCTGCTCAGACAACCACTACCTGGCGAGTATGCTCCTCC | |
| | | AGCACTCAGGGTCCCGCTCCTCTTTCCCTCCTCAGAGTTCCAACG | |
| | | AGAGGGAGAAGGTGCAGAAAGCGACTTATCTTCCCCGGCCAAAAG | |
| | | ACCACGACTCTGGCAAGAAGAGGACAGCGAGACGCAGACGCAGT | |
| | | CCTCGGAGGGGCCGGCGGAGACGACGAGGGAGCTCCTCGAGCG | |
| | | AAAGCTCAGAGAGCAGCGAGTCCTCAACCTCCAACTCCAGCAATT | |
| | | CGCCGTACAACTCGCCAAGACCCAAGCGAACCTCCACATAAACCC | |
| | | CTTATTATACTCCCAGCAGTAA | |
| AAL37162.1 | AF315077_3 | ATGCTCCTCCAGCACTCAGGGTCCCGCTCCTCTTTCCCTCCTCAG | 107 |
| | | AGTTCCAACGAGAGGGAGAAGGTGCAGAAAGCGACTTATCTTCCC | |
| | | CGGCCAAAAGACCACGACTCTGGCAAGAAGAGGACAGCGAGACG | |
| | | CAGACGCAGTCCTCGGAGGGGCCGGCGGAGACGACGAGGGAGC | |
| | | TCCTCGAGCGAAAGCTCAGAGAGCAGCGAGTCCTCAACCTCCAAC | |
| | | TCCAGCAATTCGCCGTACAACTCGCCAAGACCCAAGCGAACCTCC | |
| | | ACATAA | |
| CAF05717.1 | AJ620212.1 | ATGTACTTTTCCAGAAAAAGAAGACCCAAGAAGGAGAGGCCGCTG | 108 |
| | | CCACTGCGATACGTGTGTGGCCTACCGCCTAGCAGGCCTGATCCG | |
| | | ATGAGCTGGCGTCCACCTGCCCACGATGTCCCAGGACAAGAGGG | |
| | | CCTGTGGTACCGATCAGTTTTTACTTCTCATGGCGCTTTTTGTGGT | |
| | | TGCGGTGATTTTGTGGGTCATCTTCAGAGACTTAGCGAACGCCTG | |
| | | GGTAGACCCCAACCACCAAGACCACCGGGCGAGCCGCCGGGCCC | |
| | | TGCTGTGAGAGTTCTGCCTGCCCTGCCGCCTCCAGTACCTGAACC | |
| | | AAGAAGACACGTCCAGAGAGAGAACCCGGGATGTGGTGGTGGAG | |
| | | ACGCCGCAGATGGAGGGCCCCATGGAGAAGGAGGCGATGGAGAC | |
| | | GACGCAGACCTCGGACCAGAAGATTTAGACGAGCTGCTCGACGTC | |
| | | CTAGACGCCCCAGAGTAA | |
| CAF05718.1 | AJ620212.1 | ATGTGGTGGTGGAGACGCCGCAGATGGAGGGCCCCATGGAGAAG | 109 |
| | | GAGGCGATGGAGACGACGCAGACCTCGGACCAGAAGATTTAGAC | |
| | | GAGCTGCTCGACGTCCTAGACGCCCCAGAGTAAGGAGACCTCGG | |
| | | CGCCGCAGGGGGTGGGCTCGTAGATATAGACTTAGAAGGAGGCG | |
| | | AAGGAGGAGAAGAAGGAGAAAGCTTATACTAACACAATGGCAGCC | |
| | | AGCAAAAATAAGAAAATGTCTAGTAATAGGTTATCTTGCTCTAGTAC | |
| | | TATGTGGGAACGGGACATTCAGTAAAAACTATGCCTCCCACTCAGA | |
| | | TGACTATGTACAGAAAGGACCCTTTGGAGGGGGACTGAGCAGCAT | |
| | | GAGATTTAACATGAGAATACTATATGATCAATTTAAAAGACACCTTA | |
| | | ACTTCTGGACACACACAAACCAGGACCTAGACCTAGTTAGATACAG | |
| | | AGGCTGCACCATGACATTTTATAGACACCCAGAGGTGGACTTCATA | |
| | | GTAAAATTCAACAGAAAACCTCCATTCCTAGACACAATAGTATCAG | |
| | | GTCCAGCCATGCACCCAGGCATGCTAATGACAACAAAACACAAAA | |
| | | TACTAGTAAAAAGCTTTAAAACAAAACCCAAAGGAAAAGGCACAGT | |
| | | AAAGGTGCGCATTCGCCCCCCCACACTCTTTGACGACCGTTGGTA | |
| | | CTTTCAACATGACATCTGCAAAACCACACTGTTCACCATTAGCGCA | |
| | | ACACCATGTGACCTGCGGTTTCCGTTCTGCTCACCACAAACTGACA | |
| | | ACCCTTGCGTCAACTTCCTAGTTCTTGCAGGAGTGTATAACGGCAA | |
| | | ACTTAGCATAGAACCCACAAACGTAGAATCACAATATAATTCACTA | |
| | | CTTTCAGCTATAGAGACACACACCCAAGGCACTCTATTTAATACAT | |
| | | TTAAAACACCAGAAATGATAAAGTGCCCCCCAGCAGTAAAAGCCC | |
| | | CAGAAACTGGAGACATATCCACAAACTGCTACAAAAAACTAGACAT | |
| | | CGCCTGGGGAGACACTATATGGAACCAAAGCACCATAGGCAACTT | |
| | | TAAAAAGAACACAGAGAACTTGTGGAATGCAAGACACAATCAAACA | |
| | | ATGACTGGTAGCAAATACCTAAACTACAGAACAGGAATATACAGTG | |
| | | CCATATTCCTTTCAGCAGGCAGACTGTCACCAGACTTTCCAGGACT | |
| | | ATACAATGACATAGTATACAATCCCACCACAGACGAAGGCATAGGA | |
| | | AACATTGTGTGGATAGACTGGTGTACAAAAGCAGACTGCAACTTCA | |
| | | ATGAGACACAGTCCAAAGGAGTAATAAAAGACATTCCACTGTGGG | |
| | | CAGCACTGTTTGGCTATGTAGACTTTCTAAAAAAGACATTTAAAGA | |
| | | CGACCAGCTAGACAAAACTGCCAGACTCACTCTCATAAGCCCCTAT | |
| | | ACAAAGCCTCAACTAATAGGACCTACACAACCCAACAAAGGGTTTG | |
| | | TTCCGTACGACTACAACTTTGGCAGAGCACACATGCCCTCCGGAG | |
| | | AATCCTACATACCTATGTACTACAGATTTAGATGGTACATCTGCCTA | |
| | | TTTCACCAACAAAAGTTTATAGACGACATTGTAAGCAGCGGGCCCT | |
| | | TCGCATACCACGGCTCACAGCCCTCAGCAACTCTCACCACTAAATA | |
| | | CAAATTCCACTTTCTCTTTGGGGGCAACCCCGTTCCCCAACAGACT | |
| | | GTCAGAGACCCTTGTAACCAACCAGTCTTTGACATTCCCGGAGCC | |
| | | GGTGGACTCCCTCGTCCGATACAAGTCGTTGACCCGAAATACGTC | |
| | | AACGAAGGCTACACGTTCCACGCCTGGGACTTCCGTAGAGGGCTC | |
| | | TTTGGCCAAGCAGCTATTAAAAGAGTGTCGGGAGAACAAACAAAT | |
| | | GCTTCACTTTATTCATCAGGTCCAAAACGGCCAAGAACAGAAATTC | |
| | | CTCCAGAAAATGCAGAAGAAGGCTCATATTCCAGGGAACAAAAAC | |
| | | TCCAGCCCTGGCTCGACTCGAGCGACCAGGAAGAGAGCGAGACA | |
| | | GAAGCCCCAGAAGAAGAAGCGACCTCGCCGCCGTCGCTACAGCT | |
| | | CCAGCTCAAGCAGCAGATCAGGGAGCAGCGACAACTCAGATGTG | |
| | | GAATCCAACACCTCTTCCAGCAACTAGTGAAAACCCAGCAAAACTT | |
| | | GCATATCGACCCATGCCTACAATAG | |
| CAF05719.1 | AJ620213.1 | ATGTACTTTTCCAGAAAAAGAAGACCCAAGAAGGAGAGGCCGCTG | 110 |
| | | CCACTGCGATACGTGTGTGGCCTACCGCCTAGCAGGCCTGATCCG | |
| | | ATGAGCTGGCGTCCACCTGCCCACGATGTCCCAGGACAAGAGGG | |
| | | CCTGTGGTACCGATCAGTTTTTACTTCTCATGGCGCTTTTTGTGGT | |
| | | TGCGGTGATTTTGTGGGTCATCTTCAGAGACTTAGCGAACGCCTG | |
| | | GGTAGACCCCAACCACCAAGACCACCGGGCGGACCGCCGGGCCC | |
| | | TGCTGTGAGAGCTCTGCCTGCCCTGCCGCCTCCGGAACCTGAACC | |
| | | AAGAAGACACGTCCAGAGAGAGAACCCGGGATGTGGTGGTGGAG | |
| | | ACGCCGCAGATGGAGGGCCCCATGGAGAAGGAGGCGATGGAGAC | |
| | | GACGCAGACCTCGGACCAGAAGATTTAGACGAGCTGCTCGACGTC | |
| | | CTAGACGCCCCAGAGTAA | |
| CAF05720.1 | AJ620213.1 | ATGTGGTGGTGGAGACGCCGCAGATGGAGGGCCCCATGGAGAAG | 111 |
| | | GAGGCGATGGAGACGACGCAGACCTCGGACCAGAAGATTTAGAC | |
| | | GAGCTGCTCGACGTCCTAGACGCCCCAGAGTAAGGAGACCTCGG | |
| | | CGCCGCAGGGGGTGGGCTCGTAGATATAGACTTAGAAGGAGGCG | |
| | | GAGGAGGAGAAGAAGGAGAAAGCTTATACTAACACAATGGCAGTC | |
| | | AGCAAAAATAAGAAAATGTCTAGTAATAGGTTATCTTGCTCTAGTAC | |
| | | TATGTGGAAACGGGACATTCAGTAAAAACTATGCCTCGCACTCAGA | |
| | | TGACTATGTACAGAAAGGACCCTTTGGAGGGGGACTAAGCAGCAT | |
| | | GAGATTTAACATGAGAATACTATATGATCAATTTAAAAGACACCTTA | |
| | | ACTTCTGGACACACACAAACCAGGACCTAGACCTAGTTAGATACAG | |
| | | AGGCTGCACCATGACATTTTATAGACACCCAGAGGTGGACTTCATA | |
| | | GTAAAATTCAACAGAAAACCTCCATTCCTAGACACAATAGTATCAG | |
| | | GTCCAGCCATGCACCCAGGCATGCTAATGACAACAAAACACAAAA | |
| | | TACTAGTAAAAAGCTTTAAAACAAAACCCAAAGGAAAAGGCACAGT | |
| | | AAAGGTGCGCATTCGCCCCCCCACACTCTTTGACGACCGTTGGTA | |
| | | CTTTCAACATGACATCTGCAAAACCACACTGTTCACCATTAGCGCA | |
| | | ACACCATGTGACCTGCGGTTTCCGTTCTGCTCACCACAAACTGACA | |
| | | ACCCTTGCGTCAACTTCCTAGTTCTTGCAGGAGTGTATAACGGCAA | |
| | | ACTTAGCATAGAAGCCACAAAGTTAGAATCACAATATAATTCACTA | |
| | | GTTTCATCTATAGAAATACCCACCCAAGGCACTCTATTTAATACATT | |
| | | TAAAACACCAGAAATGATAAAGTGCCCCCCAGCAGTAAAAGCCTTA | |
| | | GAACATTCAGACGTAAACAGAAGCTGCTACAAAAAACTAGACAGC | |
| | | GCCTGGGGAGACACTATATGGAACCAGAACACCATACAGAACTTT | |
| | | AAAGAAAACACAGACAAGTTGTGGGAAGCAAGAGGCAACCAAACA | |
| | | ATGACTGGTAGCAAATACCTAAACTACAGAACAGGAATATACAGTG | |
| | | CCATATTCCTTTCAGCAGGCAGACTGTCACCAGACTTTGGGGGAC | |
| | | TATACAATGACATAGTATACAATCCCACCACAGACGAAGGCATAGG | |
| | | AAACATTGTGTGGATAGACTGGTGTACAAAAGCAGACTGCAACTTC | |
| | | AATGAGACACAGTCCAAAGGAGTAATAAAAGACATTCCACTGTGG | |
| | | GCAGCACTGTTTGGCTATGTAGACTTTCTAAAAAAGACATTTAAAG | |
| | | ACGAACAGCTAGACAAAATTGCCAGACTCACTCTCATAAGCCCCTA | |
| | | TACAAAGCCTCAACTAATAGGACCTACACAACCCAACAAAGGGTTT | |
| | | GTTCCGTACGACTACAACTTTGGCAGAGCACACATGCCCTCCGGA | |
| | | GAATCCTACATACCTATGTACTACAGATTTAGATGGTACATCTGCC | |
| | | TATTTCACCAACAAAAGTTTATAGACGACATTGTAAGCAGCGGGCC | |
| | | CTTCGCATACCACGGCTCACAGCCCTCAGCAACTCTCACCACTAA | |
| | | ATACAAATTCCACTTTCTCTTTGGGGGCAACCCCGTTCCCCAACAG | |
| | | ACTGTCAGAGACTCTTGTAACCAACCAGTCTTTGACATTCCCGGAG | |
| | | CCGGTGGACTCCCTCGTCCGATACAAGTCGTTGACCCGAAATACG | |
| | | TCAACGAAGGCTACACGTTCCACGCCTGGGACTTCCGTAGAGGGC | |
| | | TCTTTGGCCAAGCAGCTATTAAAAGAGTGTCGGGAGAACAAACAAA | |
| | | TGCTTCACTTTATTCATCAGGTCCAAAACGGCCAAGAACAGAAATT | |
| | | CCTCCACAAAATGCAGAAGAAGGCTCATATTCCAGGGAACAAAAA | |
| | | CTCCAGCCCTGGCTCGACTCGAGCGACCAGGAAGAGAGCGAGAC | |
| | | AGAAGCCCCAGAAGAAGAAGCGACCTCGCCACCGTCGCTACAGC | |
| | | TCCAGCTCAAGCAGCAGATCAGGGAGCAGCGACAACTCAGATGTG | |
| | | GAATCCAACACCTCTTCCAGCAACTAGTGAAAACCCAGCAAAACTT | |
| | | GCATATCAATCCATGCCTACAGTAG | |
| CAF05775.1 | AJ620214.1 | ATGTACTTTTCCAGAAAAAGAAGACCCAAGAAGGAGAGGCCGCTG | 112 |
| | | CCACTGCGATACGTGTGTGGCCTACCGCCTAGCAGGCCTGATCCG | |
| | | ATGAGCTGGCGTCCACCTGCCCACGATGTCCCAGGACAAGAGGG | |
| | | CCTGTGGTACCGATCAGTTTTTACTTCTCATGGCGCTTTTTGTGGT | |
| | | TGCGGTGATTTTGTGGGTCATCTTCAGAGACTTAGCGAACGCCTG | |
| | | GGTAGACCCCAACCACCAAGACCACCGGGCGGACCGCCGGGCCC | |
| | | TGCTGTGAGAGCTCTGCCTGCCCTGCCGCCTCCGGAGCCTGAAC | |
| | | CAAGAAGACACGTCCAGAGAGAGAACCCGGGATGTGGTGGTGGA | |
| | | GACGCCGCAGATGGAGGGCCCCATGGAGAAGGAGGCGATGGAG | |
| | | ACGACGCAGACCTCGGACCAGAAGATTTAGACGAGCTGCTCGACG | |
| | | TCCTAGACGCCCCAGAGTAA | |
| CAF05776.1 | AJ620214.1 | ATGTGGTGGTGGAGACGCCGCAGATGGAGGGCCCCATGGAGAAG | 113 |
| | | GAGGCGATGGAGACGACGCAGACCTCGGACCAGAAGATTTAGAC | |
| | | GAGCTGCTCGACGTCCTAGACGCCCCAGAGTAAGGAGACCTCGG | |
| | | CGCCGCAGGGGGTGGGCTCGTAGATATAGACTTAGAAGGAGGCG | |
| | | GAGGAGGAGAAGAAGGAGAAAGCTTATACTAACACAATGGCAGCC | |
| | | AGCAAAAATAAGAAAATGTCTAGTAATAGGTTATCTTGCTCTAGTAC | |
| | | TATGTGGAAACGGGACATTCAGTAAAAACTATGCCTCGCACTCAGA | |
| | | TGACTATGTACAGAAAGGACCCTTTGGAGGGGGACTAAGCAGCAT | |
| | | GAGATTTAACATGAGAATACTATATGATCAATTTAAAAGACACCTTA | |
| | | ACTTCTGGACACACACAAACCAGGACCTAGACCTAGTTAGATACAG | |
| | | AGGCTGCACCATGACATTTTATAGACACCCAGAGGTGGACTTCATA | |
| | | GTAAAATTCAACAGAAAACCTCCATTCCTAGACACAATAGTATCAG | |
| | | GTCCAGCCATGCACCCAGGCATGCTAATGACAACAAAACACAAAA | |
| | | TACTAGTAAAAAGCTTTAAAACAAAACCCAAAGGAAAAGGCACAGT | |
| | | AAAGGTACGCATTCGCCCCCCCCACACTCTTTGA | |
| CAF05777.1 | AJ620214.1 | ATGATAAAGTGCCCCCCAGCAGTAAAAGCCTTAGAACATTCAGAC | 114 |
| | | GTAAACAGAAACTGCTACAAAAAACTAGACAGCGCCTGGGGAGAC | |
| | | ACTATATGGAACCAGAACACCATACAGAACTTTAAAGAAAACACAG | |
| | | ACAAGTTGTGGGAAGCAAGAGGCAACCAAACAATGACTGGTAGCA | |
| | | AATACCTAAACTACAGAACAGGAATATACAGTGCCATATTCCTTTCA | |
| | | GCAGGCAGACTGTCACCAGACTTTGGGGGACTATACAATGACATA | |
| | | GTATACAATCCCACCACAGGCGAAGGCATAGAAAACATTGTGTGG | |
| | | ATAGACTGGTGTACAAAAGCAGACTGCAACTTCAATGAGACACAGT | |
| | | CCAAAGGAGTAATAAAAGACATTCCACTGTGGGCAGCACTGTTTG | |
| | | GCTATGTAGACTTTCTAAAAAAGACATTTAAAGACGAACAGCTAGA | |
| | | CAAAATTGCCAGACTCACTCTCATAAGCCCCTATACAAAGCCTCAA | |
| | | CTAATAGGACCTACACAACCCAACAAAGGGTTTGTTCCGTACGACT | |
| | | ACAACTTTGGCAGAGCACACATGCCCTCCGGAGAATCCTACATAC | |
| | | CTATGTACTACAGATTTAGATGGTACACCTGCCTATTTCACCAACA | |
| | | AAAGTCTATAGACGACATTGTAAGCAGCGGGCCCTTCGCATACCA | |
| | | CGGCTCACAGCCCTCAGCAACTCTCACCACTAAATACAAATTCCAC | |
| | | TTTCTCTTTGGGGGCAACCCCGTTCCCCAACAGACTGTCAGAGAC | |
| | | CCTTGTAACCAACCAATCTTTGACATTCCCGGAGCCGGTGGACTC | |
| | | CCTCGTCCGATACAAGTCGTTGACCCGAAATACGTCAACGAAGGC | |
| | | TACACGTTCCACGCCTGGGACTTCCGTAGAGGGCTCTTTGGCCAA | |
| | | GCAGCTATTAAAAGAGTGTCGGGAGAACAAACAAATGCTTCACTTT | |
| | | ATTCATCAGGTCCAAAACGGCCAAGAACAGAAATTCCTCCACAAAA | |
| | | TGCAGAAGAAGGCTCATATTCCAGGGAACAAAAACTCCAGCCCTG | |
| | | GCTCGACTCGAGCGACCAGGAAGAAAGCGAGACAGAAGCCCCAG | |
| | | AAGAAGAAGCGACCTCGCCACCGTCGCTACAGCTCCAGCTCAAGC | |
| | | AGCAGATCAGGGAGCAGCGACAACTCAGATGTGGAATCCAACACC | |
| | | TCTTCCAGCAACTAGTGAAAACCCAGCAAAACTTGCATATCAACCC | |
| | | ATGCCTACAATAG | |
| CAF05721.1 | AJ620215.1 | ATGTACTTTTCCAGAAAAAGAAGACCCAAGAAGGAGAGGCCGCTG | 115 |
| | | CCACTGCGATACGTGTGTGGCCTACCGCCTAGCAGGCCTGATCCG | |
| | | ATGAGCTGGCGTCCACCTGCCCACGATGTCCCAGGACAAGAGGG | |
| | | CCTGTGGTACCGATCAGTTTTTACTTCTCATGGCGCTTTTTGTGGT | |
| | | TGCGGTGATTTTGTGGGTCATCTTCAGAGACTTAGCGAACGCCTG | |
| | | GGTAGACCCCAACCACCAAGACCACCGGGCGGACCGCCGGGCCC | |
| | | TGCTGTGAGAGCTCTGCCTGCCCTGCCGCCTCCGGAGCCTGAAC | |
| | | CAAGAAGACACGTCCAGAGAGAGAACCCGGGATGTGGTGGTGGA | |
| | | GACGCCGCAGATGGAGGGCCCCATGGAGAAGAAGGCGATGGAGA | |
| | | CGACGCAGACCTCGGGCCAGAAGATTTAGACGAGCTGCTCGACG | |
| | | TCCTAGACGCCCCAGAGTAA | |
| CAF05722.1 | AJ620215.1 | ATGTGGTGGTGGAGACGCCGCAGATGGAGGGCCCCATGGAGAAG | 116 |
| | | AAGGCGATGGAGACGACGCAGACCTCGGGCCAGAAGATTTAGAC | |
| | | GAGCTGCTCGACGTCCTAGACGCCCCAGAGTAAGGAGACCTCGG | |
| | | CGCCGCAGGGGGTGGGCTCGTAGATATAGACTTAGAAGGAGGCG | |
| | | GAGGAGGAGAAGAAGGAGAAAGCTTATACTAACACAATGGCAGCC | |
| | | AGCAAAAATAAGAAAATGTCTAGTAATAGGTTATCTCGCTCTAGTA | |
| | | CTATGTGGAAACGGGACATTCAGTAAAAACTATGCCACGCACTCA | |
| | | GATGACTATGTACAGAAAGGACCCTTTGGAGGGGGACTAAGCAGC | |
| | | ATGAGATTTAACATGAGAATACTATATGATCAATTTAAAAGACACCT | |
| | | TAACTTCTGGACACACACAAACCAGGACCTAGACCTAGTTAGATAC | |
| | | AGAGGCTGCACCATGACATTTTATAGACACCCAGAGGTGGACTTC | |
| | | ATAGTAAAATTCAACAGAAAACCTCCATTCCTAGACACAATAGTATC | |
| | | AGGTCCAGCCATCCACCCAGGCATGCTAATGACAACAAAACACAA | |
| | | AATACTAGTAAAAAGCTTTAAAACAAAACCCAAAGGAAAAGGCACA | |
| | | GTAAAGGTGCGCATTCGCCCCCCCACACTCTTTGACGACCGTTGG | |
| | | TACTTTCAACATGACATCTGCAAAACCACACTGTTCACCATTAGCG | |
| | | CAACACCATGTGACCTGCGGTTTCCGTTCTGCTCACCACAAACTGA | |
| | | CAACCCTTGCGTCAACTTCCTAGTTCTTGCAGGAGTGTATAACGGC | |
| | | AAACTTAGCATAGAAGCCACAAAGTTAGAATCACAATATAATTCACT | |
| | | AGTTTCATCTATAGAAATACCCACCCAAGGCACTCTATTTAATACAT | |
| | | TTAAAACACCAGAAATGATAAAGTGCCCCCCAGCAGTAAAAGCCTT | |
| | | AGAACATTCAGACGTAAACAGAAACTGCTACAAAAAACTAGACAGC | |
| | | GCCTGGGGAGACACTATATGGAACCAGAACACCATACAGAACTTT | |
| | | AAAGAAAACACAGACAAGTTGTGGGAAGCAAGAGGCAACCAAACA | |
| | | ATGACTGGTAGCAAATACCTAAACTACAGAACAGGAATATACAGTG | |
| | | CCATATTCCTTTCAGCAGGCAGACTGTCACCAGACTTTGGGGGAC | |
| | | TATACAATGACATAGTATACAATCCCACCACAGACGAAGGCATAGG | |
| | | AAACATTGTGTGGATAGACTGGTGTACAAAAGCAGACTGCAACTTC | |
| | | AATGAGACACAGTCCAAAGGAGTAATAAAAGACATTCCACTGTGG | |
| | | GCAGCACTGTTTGGCTATGTAGACTTTCTAAAAAAGACATTTAAAG | |
| | | ACGAACAGCTAGACAAAATTGCCAGACTCACTCTCATAAGCCCCTA | |
| | | TACAAAGCCTCAACTAATAGGACCTACACAACCCAACAAAGGGTTT | |
| | | GTTCCGTACGACTACAACTTTGGCAGAGCACACATGCCCTCCGGA | |
| | | GAATCCTACATACCTATGTACTACAGATTTAGATGGTACATCTGCC | |
| | | TATTTCACCAACAAAAGTTTATAGACGACATTGTAAGCAGCGGGCC | |
| | | CTTCGCATACCACGGCTCACAGCCCTCAGCAACTCTCACCACTAA | |
| | | ATACAAATTCCACTTTCTCTTTGGGGGCAACCCCGTTCCCCAACAG | |
| | | ACTGTCAGAGACCCTTGTAACCAACCAGTCTTTGACATTCCCGGAG | |
| | | CCGGTGGACTCCCCCGTCCGATACAAGTCGTTGACCCGAAATACG | |
| | | TCAACGAAGGCTACACGTTCCACGCCTGGGACTTCCGTAGAGGGC | |
| | | TCTTTGGCCAAGCAGCTATTAAAAGAGTGTCGGGAGAACAAACAAA | |
| | | TGCTTCACTTTATTCATCAGGTCCAAAACGGCCAAGAACAGAAATT | |
| | | CCTCCACAAAATGCAGAAGAAGGCTCATATTCCAGGGAACAAAAA | |
| | | CTCCAGCCCTGGCTCGACTCGAGCGACCAGGAAGAGAGCGAGAC | |
| | | AGAAGCCCCAGAAGAAGAAGCGACCTCGCCACCGTCGCTACAGC | |
| | | TCCAGCTCAAGCAGCAGATCAGGGAGCAGCGACAACTCAGATGTG | |
| | | GAATCCAACACCTCTTCCAGCAACTAGTGAAAACCCAGCAAAACTT | |
| | | GCATATCAATCCATGCCTACAGTAG | |
| CAF05723.1 | AJ620216.1 | ATGTACTTTTCCAGAAAAAGAAGACCCAAGAAGGAGAGGCCGCTG | 117 |
| | | CCACTGCGATACGTGTGTGGCCTACCGCCTAGCAGGCCTGATCCG | |
| | | ATGAGCTGGCGTCCACCTGCCCACGATGTCCCAGGACAAGAGGG | |
| | | CCTGTGGTACCGATCAGTTTTTACTTCTCATGGCGCTTTTTGTGGT | |
| | | TGCGGTGATTTTGTGGGTCATCTTCAGAGACTTAGCGAACGCCTG | |
| | | GGTAGACCCCAACCACCAAGACCACCGGGCGAACCGCCGGGCCC | |
| | | TGCTGTGAGAGTTCTGCCTGCCCTGCCGCCTCCGGTACCTGAACC | |
| | | AAGAAGACACGTCCAGAGAGAGAACCCGGGATGTGGTGGTGGAG | |
| | | ACGCCGCAGATGGAGGGCCCCATGGAGAAGGAGGCGATGGAGAC | |
| | | GACGCAGACCTCGGACCAGAAGATTTAGACGAGCTGCTCGACGTC | |
| | | CTAGACGCCCCAGAGTAA | |
| CAF05724.1 | AJ620216.1 | ATGTGGTGGTGGAGACGCCGCAGATGGAGGGCCCCATGGAGAAG | 118 |
| | | GAGGCGATGGAGACGACGCAGACCTCGGACCAGAAGATTTAGAC | |
| | | GAGCTGCTCGACGTCCTAGACGCCCCAGAGTAAGGAGACCTCGG | |
| | | CGCCGCAGGGGGTGGGCTCGTAGATATAGACTTAGAAGGAGGCG | |
| | | AAGGAGGAGAAGAAGGAGAAAGCTTATACTAACACAATGGCAGCC | |
| | | AGCAAAAATAAGAAAATGTCTAGTAATAGGTTATCTTGCTCTAGTAC | |
| | | TATGTGGGAACGGGACATTCAGTAAAAACTATGCCTCCCACTCAGA | |
| | | TGACTATGTACAGAAAGGACCCTTTGGAGGGGGACTAAGCAGCAT | |
| | | GAGATTTAACATGAGAATACTATATGATCAATTTAAAAGACACCTTA | |
| | | ACTTCTGGACACACACGAACCAGGACCTAGACCTAGTTAGATACA | |
| | | GAGGCTGCACCATGACATTTTATAGACACCCAGAGGTGGACTTCA | |
| | | TAGTAAAATTCAACAGAAAACCTCCATTCCTAGACACAATAGTATCA | |
| | | GGTCCAGCCATGCACCCAGGCATGCTAATGACAACAAAACACAAA | |
| | | ATACTAGTAAAAAGCTTTAAAACAAAACCCAAAGGAAAAGGCACAG | |
| | | TAAAGGTGCGCATTCGCCCCCCCACACTCTTTGACGACCGTTGGT | |
| | | ACTTTCAACATGACATCTGCAAAACCACACTGTTCACCATTAGCGC | |
| | | AACACCATGTGACCTGCGGTTTCCGTTCTGCTCACCACAAACTGAC | |
| | | AACCCTTGCGTCAACTTCCTAGTTCTTGCAGGAGTGTATAACGGCA | |
| | | AACTTAGCATAGAACCCACAAACGTAGAATCACAATATAATTCACTA | |
| | | CTTTCAGCTATAGAGACGAACACCCAAGGCACTCTATTTAATACAT | |
| | | TTAAAACACCAGAAATGATAAAGTGCCCCGCAGCAGGAAAAGCCC | |
| | | CAGAAACTGGAGACATATCCACAAACTGCTACAAAAAACTAGACAG | |
| | | CGCCTGGGGAGACACTATATGGAACCAAAACACCATAGCCAACTT | |
| | | TAAAAAGAACACAGACAACTTGTGGAATGCAGGACACAATCAAACA | |
| | | ATGACTGGTAGCAAATACCTAAACTACAGAACAGGAATATACAGTG | |
| | | CCATATTCCTTTCAGCAGGCAGACTGTCACCAGACTTTCCAGGACT | |
| | | ATACGATGACATAGTATACAATCCCACCACAGACGAAGGCATAGG | |
| | | AAACATTGTGTGGATAGACTGGTGTACAAAAGCAGACTGCAACTTC | |
| | | AATGAGACACAGTCCAAAGGAGTAATAAAAGACATTCCACTGTGG | |
| | | GCAGCACTGTTTGGCTATGTAGACTTTCTAAAAAAGACATTTAAAG | |
| | | ACGACCAGCTAGACAAAACTGCCAGACTCACTCTCATAAGCCCCT | |
| | | ATACAAAGCCTCAACTAATAGGACCTACACAACCCAACAAAGGGTT | |
| | | TGTTCCGTACGACTACAACTTTGGCAGAGCACACATGCCCTCCGG | |
| | | AGAATCCTACATACCTATGTACTACAGATTTAGATGGTACATCTGC | |
| | | CTATTTCACCAACAAAAGTTTATAGACAACATTGTAAGCAGCGGGC | |
| | | CCTTCGCATACCACGGCTCACAGCCCTCAGCAACTCTCACCACTA | |
| | | AATACAAATTCCACTTTCTCTTTGGGGGCAACCCCGTTCCCCAACA | |
| | | GACTGTCAGAGACCCTTGTAACCAACCAGTCTTTGACATTCCCGGA | |
| | | GCCGGTGGACTCCCTCGTCCGATACAAGTCGTTGACCCGAAATAC | |
| | | GTCAACGAAGGCTACACGTTCCACGCCTGGGACTTCCGTAGAGGG | |
| | | CTCTTTGGCCAAGCAGCTATTAAAAGAGTGTCGGGAGAACAAACA | |
| | | AATGCTTCACTTTATTCATCAGGCCCAAAACGGCCAAGAACAGAAA | |
| | | TTCCTCCAGAAAATGCAGAAGAAGGCTCATATTCCAGGGAACAAAA | |
| | | ACTCCAGCCCTGGCTCGACTCGAGCGACCAGGAAGGGAGCGAGA | |
| | | CAGAAGCCCCAGAAGAAGAAGCGACCTCGCCGCCGTCGCTACAG | |
| | | CTCCAGCTCAAGCAGCAGATCAGGGAGCAGCGACAACTCAGATGT | |
| | | GGAATCCAACACCTCTTCCAGCAACTAGTGAAAACCCAGCAAAACT | |
| | | TGCATATCAACCCATGCCTACAATAG | |
| CAF05725.1 | AJ620217.1 | ATGTACTTTTCCAGAAAAAGAAGACCCAAGAAGGAGAGGCCGCTG | 119 |
| | | CCACTGCGATACGTGTGTGGCCTACCGCCTAGCAGGCCTGATCCG | |
| | | ATGAGCTGGCGTCCACCTGCCCACGATGTCCCAGGACAAGAGGG | |
| | | CCTGTGGTACCGATCAGTTTTTACTTCTCATGGCGCTTTTTGTGGT | |
| | | TGCGGTGATTTTGTGGGTCATCTTCAGAGACTTAGCGAACGCCTG | |
| | | GGTAGACCCCAACCACCAAGACCACCGGGCGGACCGCCGGGCCC | |
| | | TGCTGTGAGAGCTCTGCCTGCCCTGCCGCCTCCGGAGCCTGAAC | |
| | | CAAGAAGACACGTCCAGAGAGAGAACCCGGGATGTGGTGGTGGA | |
| | | GACGCCGCAGATGGAGGGCCCCATGGAGAAGGAGGCGATGGAG | |
| | | ACGACGCAGACCTCGGACCAGAAGATTTAGACGAGCTGCTCGACG | |
| | | TCCTAGACGCCCCAGAGTAA | |
| CAF05726.1 | AJ620217.1 | ATGTGGTGGTGGAGACGCCGCAGATGGAGGGCCCCATGGAGAAG | 120 |
| | | GAGGCGATGGAGACGACGCAGACCTCGGACCAGAAGATTTAGAC | |
| | | GAGCTGCTCGACGTCCTAGACGCCCCAGAGTAAGGAGACCTCGG | |
| | | CGCCGCAGGGGGTGGGCTCGTAGATATAGACTTAGAAGGAGGCG | |
| | | GAGGAGGAGAAGAAGGAGAAAGCTTATACTAACACAATGGCAGCC | |
| | | AGCAAAAATAAGAAAATGTCTAGTAATAGGTTATCTTGCTCTAGTAC | |
| | | TATGTGGAAACGGGACATTCAGTAAAAACTATGCCTCGCACTCAGA | |
| | | TGACTATGTACAGAAAGGACCCTTTGGAGGGGGACTAAGCAGCAT | |
| | | GAGATTTAACATGAGAGTACTATATGATCAATTTAAAAGACACCTTA | |
| | | ACTTCTGGACACACACAAACCAGGACCTAGACCTAGTTAGATACAG | |
| | | AGGCTGCACCATGACATTTTATAGACACCCAGAGGTGGACTTCATA | |
| | | GTAAAATTCAACAGAAAACCTCCATTCCTAGACACAATAGTATCAG | |
| | | GTCCAGCCATGCACCCAGGCATGCTAATGACAACAAAACACAAAA | |
| | | TACTAGTAAAAAGCTTTAAAACAAAACCCAAAGGAAAAGGCACAGT | |
| | | AAAGGTGCGCATTCGCCCCCCCACACTCTTTGACGGCCGTTGGTA | |
| | | CTTTCAACATGACATCTACAAAACCACACTGTTCACCATTAGCGCA | |
| | | ACACCGTGTGACCTGCGGTTTCCGTTCTGCTCACCACAAACTGAC | |
| | | AACCCTTGCGTCAACCTCCTAGTTCTTGCAGGAGTGTATAACGGCA | |
| | | AACTTAGCATAGAAGCCACAAAGTTAGAATCACAATATAATTCACTA | |
| | | GTTTCATCTATAGAAATACCCACCCAAGGCACTCTATTTAATACATT | |
| | | TAAAACACCAGAAATGATAAAGTGCCCCCCAGCAGTAAAAGCCTC | |
| | | AGAACATTCAGACGTAAACAGAAACTGCTACAAAAAACTAGACAGC | |
| | | GCCTGGGGAGACACTATATGGAACCCGAGCACCATACAGAACTTT | |
| | | AAAGAAAACACAGAGAAGTTGTGGGAAGCAAGAGGCAACCAAACA | |
| | | ATGACTGGTAGCAAATACCTAAACTACAGAACAGGAATATACAGTG | |
| | | CCATATTCCTTTCAGCAGGCAGACTGTCACCAGACTTTGGGGGAC | |
| | | TATACAATGACATAGTATACAATCCCACCACAGACGAAGGCATAGG | |
| | | AAACATTGTGTGGATAGACTGGTGTACAAAAGCAGACTGCAACTTC | |
| | | AATGAGACACAGTCCAAAGGGGTAATAAAAGACATTCCACCGTGG | |
| | | GCAGCACTGTTTGGCTATGTAGACTTTCTAAAAAAGACATTTAAAG | |
| | | ACGAACAGCTAGACAAAATTGCCAGACTCACTCTCATAAGCCCCTA | |
| | | TACAAAGCCTCAACTAATAGGACCTACACAACCCAACAAAGGGTTT | |
| | | GTTCCGTACGACTACAACTTTGGCAGAGCACACATGCCCTCCGGA | |
| | | GAATCCTACATACCTATGTACTACAGATTTAGATGGTACATCTGCC | |
| | | TATTTCACCAACAAAAGTTTATAGACGACATTGTAAGCAGCGGGCC | |
| | | CTTCGCATACCACGGCTCACAGCCCTCAGCAACTCTCACCACTAA | |
| | | ATACAAATTCCACTTTCTCTTTGGGGGCAACCCCGTTCCCCAACAG | |
| | | ACTGTCAGAGACCCTTGTAACCAACCAGTCTTTGACATTCCCGGAG | |
| | | CCGGTGGACTCCCTCGTCCGATACAAGTCGTTGACCCGAAATACG | |
| | | TCAACGAAGGCTACACGTTCCACGCCTGGGACTTCCGTAGAGGGC | |
| | | TCTTTGGCCAAGCAGCTATTAAAAGAGTGTCGGGAGAACAAACAAA | |
| | | TGCTTCACTTTATTCATCAGGTCCAAAACGGCCAAGAACAGAAATT | |
| | | CCTCCACAAAATGCAGAAGAAGGCTCATATTCCAGGGAACAAAAA | |
| | | CTCCAGCCCTGGCTCGACTCGAGCGACCAGGAAGAGAGCGAGAC | |
| | | AGAAGCCCCAGAAGAAGAAGCGACCTCGCCACCGTCGCTACAGC | |
| | | TCCAGCTCAAGCAGCAGATCAGGGAGCAGCGACAACTCAGATGTG | |
| | | GAATCCAACACCTCTTCCAGCAACTAGTGAAAACCCAGCAAAACTT | |
| | | GCATATCAACCCATGCCTACAATAG | |
| CAF05727.1 | AJ620218.1 | ATGCGTTTTCGCAGGGTTGCCCAGAAAAGGAAAGTGCTTTTGCAA | 121 |
| | | ACTGTGCCAGCTGCAAAGAAGGCTAGGCGGCTTCTAGGTATGTGG | |
| | | CAGCCCCCCACGCACAATGTCCCGGGCATCGAGAGAAACTGGTA | |
| | | CGAGAGCTGTTTTAGATCCCACGCTGCTGTTTGTGGCTGTGGCGA | |
| | | TTTTGTTGGCCATCTTAATCATCTGGCAACTACTCTGGGTCGTCCT | |
| | | CCGCGTCCTGGGCCCCCAGGCGGACCCCGCACGCCGCAAATAAG | |
| | | AAACCTGCCAGCGCTCCCGGCGCCCCAGGGCGAGCCCGGTGACA | |
| | | GAGCGTCATGGCGTGGGGCTTCTGGGGCCGACGCCGCCGGTGG | |
| | | AGACGATGGAGAGCGCGGCGCAGACGGTGGAGACCCCGCAGAC | |
| | | GTAGGAGACGACGCCCTCCTCGCCGCTTTCGAGCTCGTCGAAGA | |
| | | GTAA | |
| CAF05728.1 | AJ620218.1 | ATGGCGTGGGGCTTCTGGGGCCGACGCCGCCGGTGGAGACGAT | 122 |
| | | GGAGAGCGCGGCGCAGACGGTGGAGACCCCGCAGACGTAGGAG | |
| | | ACGACGCCCTCCTCGCCGCTTTCGAGCTCGTCGAAGAGTAAGGAG | |
| | | GCGCGGGGGGAGGTGGCGCAGACGCTACAGAAAATGGCGACGG | |
| | | GGCAGACGCAGACGGACTCACAGAAAAAAGATAGTCATAAAACAG | |
| | | TGGCAACCTAACTTTATAAGACGCTGCTACATCATAGGGTACTTAC | |
| | | CACTTATATTCTGCGGCGAAAATACAACCGCCCAGAACTTTGCCAC | |
| | | TCACTCGGACGACATGATAAGCAAAGGACCGTACGGGGGGGGCA | |
| | | TGACTACCACCAAATTCACTCTGAGAATACTGTACGACGAGTTTAC | |
| | | CAGGTTTATGAACTTTTGGACTGTCAGTAACGAAGACCTAGACCTG | |
| | | TGTAGATACGTGGGCTGCAAACTAATATTTTTTAAACACCCCACGG | |
| | | TGGACTTTATAGTACAGATAAACACTCAGCCTCCTTTCTTAGACAC | |
| | | GCACCTCACCGCGGCCAGCATACACCCGGGCATCATGATGCTCA | |
| | | GCAAGAGACACATACTAATACCCTCTCTAAAGACCCGGCCCAGCA | |
| | | GAAAACACAGGGTGGTCGTCAGGGTGGGCGCCCCAAGACTTTTTC | |
| | | AGGACAAGTGGTACCCCCAGTCAGACCTGTGTGACACAGTTCTGC | |
| | | TTTCCATATTCGCAACCGCCTGCGACTTGCAATATCCGTTCGGCTC | |
| | | ACCACTAACTGACAACCCTTGCGTCAACTTCCAGATCCTGGGGCC | |
| | | CCAGTACAAAAAACACCTTAGTATTAGCTCCACTATGGATGACACT | |
| | | AACAAAGCACATTATGAAGAAAACTTATTTAAGAAAATTGAACTATA | |
| | | CAACACCTTTCAAACCATAGCTCAGCTTAAAGAGACAGGAACAATT | |
| | | TCAGGCATGCAACCTTCTTGGACTGAAGTCCAGAATTCAAAAACAC | |
| | | TTAATGAAACAGGTAGCAATGCCACTGAGAGTAGAGACACTTGGTA | |
| | | TAAAGGAAATACATACAACGACAAGATACACCAGTTAGCAGAAAAA | |
| | | ACCAGAAAGAGATTTAAAAATGCAACAAAAGCAGCACTACCAAACT | |
| | | ACCCCACAATAATGTCCGCAGACTTATATGAATACCACTCAGGCAT | |
| | | ATACTCCAGCATATATCTATCAGCTGGCAGGAGCTACTTTGAAACC | |
| | | ACCGGGGCCTACTCTGACATTATATACAACCCTTTCACAGACAAGG | |
| | | GCACAGGCAACATAATCTGGATAGACTACCTCACAAAAGAAGACA | |
| | | CCATTTTTGTAAAAAACAAAAGCAAATGCGAGATAATGGACATGCC | |
| | | CCTGTGGGCGGCCTGCACAGGATACACAGAGTTTTGTGCAAAGTA | |
| | | TACAGGCGACTCTGCCATTATTTACAATGCAAGAATAGTCATAAGA | |
| | | TGCCCATACACTGAGCCCATGTTAATAGACCACTCAGACCCAAACA | |
| | | AAGGCTTCGTTCCCTACTCATTTAGCTTTGGCAACGGAAAGATGCC | |
| | | CGGAGGCAGCTCCAACGTGCCCATAAGAATGAGAGCCAAGTGGTA | |
| | | CGTGAACATATTCCACCAAAAAGAAGTATTGGAGAGCATAGTACAG | |
| | | TCCGGACCGTTTGGGTACAAGGGCGACATAAAATCAGCTGTACTA | |
| | | GCCATGAAATACAGATTTCACTGGAAGTGGGGCGGAAACCCTATA | |
| | | TCCAAACAGGTCGTCAGGAATCCCTGCTCCAACTCCAGCTCATCC | |
| | | GCGGCCCATAGAGGACCTCGCAGCGTACAAGCGGTTGACCCGAA | |
| | | ATACAATACCCCAGAGGTCACGTGGCACTCGTGGGACATTAGACG | |
| | | AGGACTCTTTGGCAAAGCAGGTATTAAAAGAATGCAACAGGAATCA | |
| | | GATGCTCTTTACATTCCTCCAGGACCAATCAAGAGACCTCGCAGG | |
| | | GACACCAACGCCCAAGACCCAGAAGAGCAAAACGAAAGCTCAGGT | |
| | | TTCAGAGTCCAGCAGCGACTCCCGTGGGTCCACTCCAGCCAAGAG | |
| | | ACGCAAAGCTCCCAAGAAGAGACGGAGGCGCAGGGGTCGGTACA | |
| | | AGACCAACTACTCCTCCAGCTCCGAGAGCAGCGAGTTCTCCGACT | |
| | | CCAGCTCCAGCAACTCGCAACCCAAGTCCTCAAAGTCCAAGCAGG | |
| | | GCACAGCCTACACCCCCTATTATCTTCCCAAGCATAA | |
| CAF05729.1 | AJ620219.1 | ATGCGTTTTCGCAGGGTTGCCCAGAAAAGGAAAGTGCTTTTGCAA | 123 |
| | | ACTGTGCCAGCTGCAAAGAAGGCTAGGCGGCTTCTAGGTATGTGG | |
| | | CAGCCCCCCACGCATAATGTCCCGGGCATCGAGAGAAACTGGTAC | |
| | | GAGAGCTGTTTTAGATCCCACGCTGCTGTTTGTGGCTGTGGCGAT | |
| | | TTTGTTGGCCATCTTAATCATCTGGCAACTACTCTGGGTCGTCCTC | |
| | | CGCGTCCTGGGCCCCCAGGCGGACCCCGCACGCCGCAAATAAGA | |
| | | AACCTGCCAGCGCTCCCGGCGCCCCAGGGCGAGCCCGGTGACA | |
| | | GAGCGTCATGGCGTGGGGCTTCTGGGGCCGACGCCGCCGGTGG | |
| | | AGACGATGGAGAGCGCGGCGCAGACGGTGGAGACCCCGCAGAC | |
| | | GTAGGAGACGACGCCCTCCTCGCCGCTTTCGAGCTCGTCGAAGA | |
| | | GTAA | |
| CAF05730.1 | AJ620219.1 | ATGGCGTGGGGCTTCTGGGGCCGACGCCGCCGGTGGAGACGAT | 124 |
| | | GGAGAGCGCGGCGCAGACGGTGGAGACCCCGCAGACGTAGGAG | |
| | | ACGACGCCCTCCTCGCCGCTTTCGAGCTCGTCGAAGAGTAAGGAG | |
| | | GCGCGGGGGGAGGTGGCGCAGACGCTACAGAAAATGGCGACGG | |
| | | GGCAGACGCAGACGGACTCACAGAAAAAAGATAGTCATAAAACAG | |
| | | TGGCAACCTAACTTTATAAGACGCTGCTACATCATAGGGTACTTAC | |
| | | CACTTATATTCTGCGGCGAAAATACAACCGCCCAGAACTTTGCCAC | |
| | | TCACTCGGACGACATGATAAGCAAAGGACCGTACGGGGGGGGCA | |
| | | TGACTACCACCAAATTCACTCTGAGAATACTGTACGACGAGTTTAC | |
| | | CAGGTTTATGAACTTTTGGACTATCAGTAACGAAGACCTAGACCTG | |
| | | TGTAGATACGTGGGCTGCAAACTAATATTTTTTAAACACCCCACGG | |
| | | TGGACTTTATAGTACAGATAAACACTCAGCCTCCTTTCTTAGACAC | |
| | | GCACCTCACCGCGGCCAGCATACACCCGGGCATCATGATGCTCA | |
| | | GCAAGAGACACATACTAATACCCTCTCTAAAGACCCGGCCCAGCA | |
| | | GAAAACACAGGGTGGTCGTCAGGGTGGGCGCCCCAAGACTTTTTC | |
| | | AGGACAAGTGGTACCCCCAGTCAGACCTGTGTGACACAGTTCTGC | |
| | | TTTCCATATTCGCAACCGCCTGCGACTTGCAATATCCGTTTGGCTC | |
| | | ACCACTAACTGACAACCCTTGCGTCAACTTCCAGATCCTGGGGCC | |
| | | CCAGTACAAAAAACACCTTAGTATTAGCTCCACTATGGATGAAAGT | |
| | | AACATATCACATTATAAAGAAAACTTATTTAAGAAAACTGAACTATA | |
| | | CAACACCTTTCAAACCATAGCTCAGCTTAAAGAGACAGGAAACATT | |
| | | TCAGGCATTAGTCCTAATTGGACTGAAGTCCAGAATTCAACAACAC | |
| | | TTAATCAAACAGGTGACAATGCCACTAACAGTAGAGACACTTGGTA | |
| | | TAAAGGAAATACATACAACCACAAGATATGCGACTTAGCAGAAAAA | |
| | | ACCAGAAACAGATTTAAAAATGCAACCAAAGCAGCACTACCAAACT | |
| | | ACCCCACAATAATGTCCACAGACCTATATGAATACCACTCAGGCAT | |
| | | ATACTCCAGCATATATTTATCAGCTGGCAGGAGCTACTTTGAAACC | |
| | | ACCGGGGCCTACTCTGACATTATATACAACCCTTTCACAGACAAAG | |
| | | GCACAGGCAACATAATCTGGATAGACTACCTCACAAAAGAAGACA | |
| | | CCATTTTTGTAAAAAACAAAAGCAAATGCGAGATAATGGACATGCC | |
| | | CCTGTGGGCGGCCTGCACAGGATACACAGAGTTTTGTGCAAAGTA | |
| | | TACAGGCGACTCTGCCATTATCTACAATGCAAGAATACTCATAAGA | |
| | | TGCCCATACACTGAGCCCATGTTAATAGACCACTCAGACCCAAACA | |
| | | AAGGCTTCGTTCCCTACTCATTTAACTTTGGCAACGGAAAGATGCC | |
| | | CGGAGGCAGCTCCAACGTACCCATAAGAATGAGAGCCAAATGGTA | |
| | | CGCGAACATATTCCACCAAAAGGAGGTTCTAGAGGCTATAGTACAA | |
| | | AGCGGACCGTTCGGGTACAAGGGCGACATAAAATCAGCTGTACTA | |
| | | GCCATGAAATACAGATTTCACTGGAAGTGGGGCGGAAACCCTATA | |
| | | TCCAAACAGGTCGTCAGGAATCCCTGCTCCAACTCCAGCTCATCC | |
| | | GCGGCCCATAGAGGACCTCGCAGCGTACAAGCGGTTGACCCGAA | |
| | | ATACAATACCCCAGAGGTCACGTGGCACTCGTGGGACATTAGACG | |
| | | AGGACTCTTTGGCAAAGCAGGTATTAAAAGAATGCAACAGGAATCA | |
| | | GATGCTCTTTACATTCCTCCAGGACCAATCAAGAGACCTCGCAGG | |
| | | GACACCAACGCCCAAGACCCAGAAGAGCAAAACGAAAGCTCAGGT | |
| | | TTCAGAGTCCAGCAGCGACTCCCGTGGGTCCACTCCAGCCAAGAG | |
| | | ACGCAAAGCTCCCAAGAAGAGACGGAGGCGCAGGGGTCGGTACA | |
| | | AGACCAACTACTCCTCCAGCTCCGAGAGCAGCGAGTTCTCCGACT | |
| | | CCAGCTCCAGCAACTCGCAGCCCAAGTCCCCAAAGTCCAAGCAGG | |
| | | GCACAGCCTACACCCCCTATTATCTTCCCAAGCATAA | |
| CAF05731.1 | AJ620220.1 | ATGCGTTTTCGCAGGGTTGCCCAGAAAAGGAAAGTGCTTTTGCAA | 125 |
| | | ACTGTGCCAGCTGCAAAGAAGGCTAGGCGGCTTCTAGGTATGTGG | |
| | | CAGCCCCCCACGCACAATGTCCCGGGCATCGAGAGAAACTGGTA | |
| | | CGAGAGCTGTTTTAGATCCCACGCTGCTGTTTGTGGCTGTGGCGA | |
| | | TTTTGTTGGCCATCTTAATCATCTGGCAACTACTCTGGGTCGTCCT | |
| | | CCGCGTCCTGGGCCCCCAGGCGGACCCCGCACGCCGCAAATAAG | |
| | | AAACCTGCCAGCGCTCCCGGCGCCCCAGGGCGAGCCCGGTGACA | |
| | | GAGCGTCATGGCGTGGGGCTTCTGGGGCCGACGCCGCCGGTGG | |
| | | AGACGATGGAGAGCGCGGCGCAGACGGTGGAGACCCCGCAGAC | |
| | | GTAGGAGACGACGCCCTCCTCGCCGCTTTCGAGCTCGTCGAAGA | |
| | | GTAA | |
| CAF05732.1 | AJ620220.1 | ATGGCGTGGGGCTTCTGGGGCCGACGCCGCCGGTGGAGACGAT | 126 |
| | | GGAGAGCGCGGCGCAGACGGTGGAGACCCCGCAGACGTAGGAG | |
| | | ACGACGCCCTCCTCGCCGCTTTCGAGCTCGTCGAAGAGTAAGGAG | |
| | | GCGCGGGGGGAGGTGGCGCAGACGCTACAGAAAATGGCGACGG | |
| | | GGCAGACGCAGACGGACTCACAGAAAAAAGATAGTCATAAAACAG | |
| | | TGGCAACCAAACTTTATAAGACGCTGCTACATCATAGGGTACTTAC | |
| | | CACTTATATTCTGCGGCGAAAATACAACCGCCCAGAACTTTGCCAC | |
| | | TCACTCGGACGACATGATAAGCAAAGGACCGTACGGGGGGGGCA | |
| | | TGACTACCACCAAATTCACTCTGAGAATACTGTACGACGAGTTTAC | |
| | | CAGGTTTATGAACTTTTGGACTGTCAGTAACGAAGACCTAGACCTG | |
| | | TGTAGATACGTGGGCTGCAAACTAATATTTTTTAAACACCCCACGG | |
| | | TGGACTTTATAGTACAGATAAACACTCAGCCTCCTTTCTTAGACAC | |
| | | GCACCTCACCGCGGCCAGCATACACCCGGGCATCATGATGCTCA | |
| | | GCAAGAGACACATACTAATACCCTCTCTAAAGACCCGGCCCAGCA | |
| | | GAAAACACAGGGTGGTCGTCAGGGTGGGCGCCCCAAGACTTTTTC | |
| | | AGGACAAGTGGTACCCCCAGTCAGACCTGTGTGACACAGTTCTGC | |
| | | TTTCCATATTCGCAACCGCCTGCGACTTGCAATATCCGTTCGGCTC | |
| | | ACCACTAACTGACAACCCTTGCGTCAACTTCCAGATCCTGGGGCC | |
| | | CCAGTACAAAAAACACCTTAGTATTAGCTCCACTATGGATGACACT | |
| | | AACAAAGCACATTATGAAGAAAACTTATTTAATAAAACTGAACTATA | |
| | | CAACACCTTTCAAACCATAGCTCAGCTTAGAGACACAGGACAAACT | |
| | | ACAAACGCTAGTCCTAATTGGAATCAGGTCCAGAATACAGCAGCA | |
| | | CTTGAGTTATCAGGTGCAAATGCCACTAGCAGCAAAGACACTTGGT | |
| | | ATAAAGGTAATACATACACGAAAGACATATCAAAGTTAGCAGAAAA | |
| | | AACCAGACAAAGATTTAAAGCTGCAACAATAGCAGCACTACCAAAC | |
| | | TACCCCACAATAATGTCCACAGACCTATATGAATACCACTCAGGCA | |
| | | TATACTCCAGCATATATTTATCAGCTGGCAGGAGCTACTTTGAAAC | |
| | | CACCGGGGCCTACTCTGACATTATATACAACCCTTTCACAGACAAA | |
| | | GGCACAGGCAACATAATCTGGATAGACTACCTCACAAAAGAAGAC | |
| | | ACCATTTTTGTAAAAAACAAAAGCAAATGCGAGATAATGGACATGC | |
| | | CCCTGTGGGCGGCCTGCACAGGATACACAGAGTTTTGTGCAAAGT | |
| | | ATACAGGCGACTCTGCCATTATCTACAATGCAAGAATACTCATAAG | |
| | | ATGCCCACACACTGAGCCCATGTTAATAGACCACTCAGACCCAAA | |
| | | CAAAGGCTTCGTTCCCTACTCATTCGACTTTGGCAATGGAAAGATG | |
| | | CCCGGAGGCAGCTCCAACGTACCGATAAGAATGAGGGCCAAATG | |
| | | GTACGTGAACATATTCCACCAAAAGGAGGTTCTAGAGGCTATAGTA | |
| | | CAAAGCGGACCGTTCGGGTACAAGGGCGACATAAAATCAGCTGTA | |
| | | CTAGCCATGAAATACAGATTTCACTGGAAGTGGGGCGGAAACCCT | |
| | | ATATCCAAACAGGTCGTCAGGAATCCCTGCTCCAACTCCAGCTCAT | |
| | | CCGCGGCCCATAGAGGACCTCGCAGCGTACAAGCGGTTGACCCG | |
| | | AAATACAATACCCCAGAGGTCACGTGGCACTCGTGGGACATTAGA | |
| | | CGAGGACTCTTTGGCAAAGCAGGTATTAAAAGAATGCAACAGGAA | |
| | | TCAGATGCTCTTTACATTCCTCCAGGACCAATCAAGAGACCTCGCA | |
| | | GGGACACCAACGCCCAAGACCCAGAAGAGCAAAACGAAAGCTCA | |
| | | GGTTTCAGAGTCCAGCAGCGACTCCCGTGGGTCCACTCCAGCCAA | |
| | | GAGACGCAAAGCTCCCAAGAAGAGACGGAGGCGCAGGGGTCGGT | |
| | | ACAAGACCAACTACTCCTCCAGCTCCGAGAGCAGCGAGTTCTCCG | |
| | | ACTCCAGCTCCAGCAACTCGCAACCCAAGTCCTCAAAGTCCAAGC | |
| | | AGGGCACAGCCTACACCCCCTATTATCTTCCCAAGCATAA | |
| CAF05733.1 | AJ620221.1 | ATGCGTTTTCGCAGGGTTGCCCAGAAAAGGAAAGTGCTTTTGCAA | 127 |
| | | ACTGTGCCAGCTGCAAAGAAGGCTAGGCGGCTTCTAGGTATGTGG | |
| | | CAGCCCCCCACGCACAATGTCCCGGGCATCGAGAGAAACTGGTA | |
| | | CGAGAGCTGTTTTAGATCCCACGCTGCTGTTTGTGGCTGTGGCGA | |
| | | TTTTGTTGGCCATCTTAATCATCTGGCAACTACTCTGGGTCGTCCT | |
| | | CCGTGTCCTGGGCCCCCAGGCGGACCCCGCACGCCGCAAATAAG | |
| | | AAACCTGCCAGCGCTCCCGGCGCCCCAGGGCGAGCCCGGTGACA | |
| | | GAGCGCCATGGCGTGGGGCTTCTGGGGCCGACGCCGCCGGTGG | |
| | | AGACGATGGAGAGCGCGGCGCAGACGGTGGAGACCCCGCAGAC | |
| | | GTAGGAGACGACGCCCTACTCGCCGCTTTCGAGCTCGTCGAAGA | |
| | | GTAA | |
| CAF05734.1 | AJ620221.1 | ATGGCGTGGGGCTTCTGGGGCCGACGCCGCCGGTGGAGACGAT | 128 |
| | | GGAGAGCGCGGCGCAGACGGTGGAGACCCCGCAGACGTAGGAG | |
| | | ACGACGCCCTACTCGCCGCTTTCGAGCTCGTCGAAGAGTAAGGAG | |
| | | GCGCGGGGGGAGGTGGCGCAGACGCTACAGAAAATGGCGACGG | |
| | | GGCAGACGCAGACGGACTCATAGAAAAAAGATAGTCATAAAACAG | |
| | | TGGCAACCAAACTTTATAAGACGCTGCTACATCATAGGGTACTTAC | |
| | | CACTTATATTCTGCGGCGAAAATACAACCGCCCAGAACTTTGCCAC | |
| | | TCGCTCGGACGACATGATAAGCAAAGGACCGTACGGGGGGGGCA | |
| | | TGACTACCACCAAATTCACTCTGAGAATACTGTACGACGAGTTTAC | |
| | | CAGGTTTATGAACTTTTGGACTGTCAGTAACGAAGACCTAGACCTG | |
| | | TGTAGATACGTGGGCTGCAAACTAATATTTTTTAAACACCCCACGG | |
| | | TGGACTTTATAGTACAGATAAACACTCAGCCTCCTTTCTTAGACAC | |
| | | GCACCTCACCGCGGCCAGCATACACCCGGGCATCATGATGCTCA | |
| | | GCAAGAGACACATACTAATACCCTCTCTAAAGACCCGGCCCAGCA | |
| | | GAAAACACAGGGTGGTCGTCAGGGTGGGCGCCCCAAGACTTTTTC | |
| | | AGGACAAGTGGTACCCCCAGTCAGACCTGTGTGACACAGTTCTGC | |
| | | TTTCCATATTCGCAACCGCCTGCGACTTGCAATATCCGTTCGGCTC | |
| | | ACCACTAACTGACAACCCTTGCGTCAACTTCCAGATCCTGGGGCC | |
| | | CCAGTACAAAAAACACCTTAGTATTAGCTCCACTATGGATGAAAGT | |
| | | AACAAAGCACATTATGAACAAAACTTATTTAAGAAAACTGAACTATA | |
| | | CAACACCTTTCAAACCATAGCTCAGCTTAAAGAGACAGGAAACATT | |
| | | TCAGGCATTACTCCTACTTGGACTGAAGTCCAGAATTCAACAACAC | |
| | | TTAATCAAGCAGGTAACAATGCCACTGACAGTAGAGACACTTGGTA | |
| | | TAAAGGAAATACATACAACGAGAAGATATCCGAGTTAGCACAAATA | |
| | | ACCAGAAACAGATTTAAAAATGCAACCAAAACAGCACTACCAAACT | |
| | | ACCCCACAATAATGTCCACAGACCTATATGAATACCACTCAGGCAT | |
| | | ATACTCCAGCATATATTTATCAGCTGGCAGGAGCTACTTTGAAACC | |
| | | ACCGGGGCCTACTCTGACATTATATACAACCCTTTCACAGACAAAG | |
| | | GCACAGGCAACATAATCTGGATAGACTACCTCACAAAAGAAGACA | |
| | | CCATTTTTGTAAAAAACAAAAGCAAATGCGAGATAATGGACATGCC | |
| | | CCTGTGGGCGGCCTGCACAGGATACACAGAGTTTTGTGCAAAGTA | |
| | | TACAGGCGACTCTGCCATTATTTACAATGCAAGAATAGTCATAAGA | |
| | | TGCCCATACACTGAGCCCATGTTAATAGACCACTCAGACCCAAACA | |
| | | AAGGCTTCGTCCCCTACTCATTTAACTTTGGCAACGGAAAGATGCC | |
| | | CGGAGGCAGCTCCAACGTGCCCATAAGAATGAGAGCCAAGTGGTA | |
| | | CGTGAACATATTCCACCAAAAAGAAGTATTGGAGAGCATAGTACAG | |
| | | TCCGGACCGTTTGGGTACAAGGGCGACATAAAATCAGCTGTACTA | |
| | | GCCATGAAATACAGATTTCACTGGAAGTGGGGCGGAAACCCTATA | |
| | | TCCAAACAGGTCGTCAGGAATCCCTGCTCCAACTCCAGCTCATCC | |
| | | GCGGCCCATAGAGGACCTCGCAGCGTACAAGCGGTTGACCCGAA | |
| | | ATACAATACCCCAGAGGTCACGTGGCACTCGTGGGACATTAGACG | |
| | | AGGACTCTTTGGCAAAGCAGGTATTAAAAGAATGCAACAGGAATCA | |
| | | GATGCTCTTTACATTCCTCCAGGACCAATCAAGAGACCTCGCAGG | |
| | | GACACCAACGCCCAAGACCCAGAAGAGCAAAACGAAAGCTCAGGT | |
| | | TTCAGAGTCCAGCAGCGACTCCCGTGGGTCCACTCCAGCCAAGAG | |
| | | ACGCAAAGCTCCCAAGAAGAGACGGAGGCGCAGGGGTCGGTACA | |
| | | AGACCAACTACTCCTCCAGCTCCGAGAGCAGCGAGTTCTCCGACT | |
| | | CCAGCTCCAGCAACTCGCAACCCAAGTCCTCAAAGTCCAAGCAGG | |
| | | GCACAGCCTACACCCCCTATTATCTTCCCAAGCATAA | |
| CAF05735.1 | AJ620222.1 | ATGCGTTTTCGCAGGGTTGCCCAGAAAAGGAAAGTGCTTTTGCAA | 129 |
| | | ACTGTGCCAGCTGCAAAGAAGGCTAGGCGGCTTCTAGGTATGTGG | |
| | | CAGCCCCCCACGCACAATGTCCCGGGCATCGAGAGAAACTGGTA | |
| | | CGAGAGCTGTTTTAGATCCCACGCTGCTGTTTGTGGCTGTGGCGA | |
| | | TTTTGTTGGCCATCTTAATCATCTGGCAACTACTCTGGGTCGTCCT | |
| | | CCGCGTCCTGGGCCCCCAGGCGGACCCCGCACGCCGCAAATAAG | |
| | | AAACCTGCCAGCGCTCCCGGCGCCCCAGGGCGAGCCCGGTGACA | |
| | | GAGCGCCATGGCATGGGGCTTCTGGGGCCGACGCCGCCGGTGG | |
| | | AGACGATGGAGAGCGCGGCGCAGACGGTGGAGACCCCGCAGAC | |
| | | GTAGGAGACGACGCCCTACTCGCCGCTTTCGAGCTCGTCGAAGA | |
| | | GTAA | |
| CAF05736.1 | AJ620222.1 | ATGGCATGGGGCTTCTGGGGCCGACGCCGCCGGTGGAGACGATG | 130 |
| | | GAGAGCGCGGCGCAGACGGTGGAGACCCCGCAGACGTAGGAGA | |
| | | CGACGCCCTACTCGCCGCTTTCGAGCTCGTCGAAGAGTAAGGAG | |
| | | GCGCGGGGGGAGGTGGCGCAGACGCTACAGAAAATGGCGACGG | |
| | | GGCAGACGCAGACGGACTCATAGAAAAAAGATAGTCATAAAACAG | |
| | | TGGCAACCAAACTTTATAAGACGCTGCTACGTCATAGGGTACTTAC | |
| | | CACTTATATTCTGCGGCGAAAATACAACCGCCCAGAACTTTGCCAC | |
| | | TCACTCGGACGACATGATAAGCAAAGGACCGTACGGGGGGGGCA | |
| | | TGACTACCACCAAATTCACTCTGAGAATACTGTACGACGAGTTTAC | |
| | | CAGGTTTATGAACTTTTGGACTGTCAGTAACGAAGACCTAGACCTG | |
| | | TGTAGATACGTGGGCTGCAAACTAATATTTTTTAAACACCCCACGG | |
| | | TGGACTTTATAGTACAGATAAACACTCAGCCTCCTTTCTTAGACAC | |
| | | GCACCTCACCGCGGCCAGCATACACCCGGGCATCATGATGCTCA | |
| | | GCAAGAGACACATACTAATACCCTCTCTAAAGACCCGGCCCAGCA | |
| | | GAAAACACAGGGTGGTCGTCAGGGTGGGCGCCCCAAGACTTTTTC | |
| | | AGGACAAGTGGTACCCCCAGTCAGACCTGTGTGACACAGTTCTGC | |
| | | TTTCCATATTTGCAACCGCCTGCGACTTGCAATATCCGTTCGGCTC | |
| | | ACCACTAACTGACAACCCTTGCGTCAACTTCCAGATCCTGGGGCC | |
| | | CCAGTACAAAAAACACCTTAGTATTAGCTCCACTATGGATCAAACT | |
| | | AACGAAAACCATTATAAAGAAAACTTATTTAACAAAACTGAACTATA | |
| | | CAACACCTTTCAAACCATAGCTCAGCTTAAAGAGACAGGACACATT | |
| | | TCAGGCATTAGTCCTACTTGGAATGAAGTCCAGAATTCAACAACAC | |
| | | TTACTAAAGGAGGTGACAATGCCACTCAGAGTAGAGACACTTGGT | |
| | | ATAAAGGAAATACATACAACGAGAAGATATGCGAGTTAGCACAAAT | |
| | | AACCAGAAACAGATTTAAAAATGCAACCAAAGGAGCACTACCAAAC | |
| | | TACCCCACAATAATGTCCACAGACCTATATGAATACCACTCAGGCA | |
| | | TACACTCCAGCATATATCTATCAGCTGGCAGGAGCTACTTTGAAAC | |
| | | CACCGGGGCCTACTCTGACATTATATACAACCCTTTCACAGACAAA | |
| | | GGCACAGGCAACATAATCTGGATAGACTACCTCACAAAAGAAGAC | |
| | | ACCATTTTTGTGAAAAACAAAAGCAAATGCGAGATAATGGACATGC | |
| | | CCCTGTGGGCGGCCTGCACAGGATACACAGAGTTTTGTGCAAAGT | |
| | | ATACAGGCGACTCTGCCATTATCTACAATGCAAGAATACTCATAAG | |
| | | ATGCCCATACACTGAGCCCATGTTAATAGACCACTCAGACCCAAAC | |
| | | AAAAGCTTCGTTCCCTACTCATTTAACTTTGGCAACGGAAAGATGC | |
| | | CCGGAGGCAGCTCCAACGTGCCCATAAGAATGAGAGCCAAGTGG | |
| | | TACGTGAACATATTCCACCAAAAAGAAGTATTAGAGAGCATAGTAC | |
| | | AGTCCGGACCGTTTGGGTACAAGGGCGACATAAGATCAGCTGTAC | |
| | | TAGCCATGAAATACAGATTTCACTGGAAGTGGGGCGGAAACCCTA | |
| | | TATCCAAACAGGTCGTCAGGAATCCCTGCTCCAACTCCAGCTCCT | |
| | | CCGCGGCCCATAGAGGACCTCGCAGCGTACAAGCGGTTGACCCG | |
| | | AAATACAATACCCCAGAGGTCACGTGGCACTCGTGGGACATTAGA | |
| | | CGAGGACTCTTTGGCAAAGCAGGTATTAAAAGAATGCAACAGGAA | |
| | | TCAGATGCTCTTTACATTCCTCCAGGACCAATCAAGAGACCTCGCA | |
| | | GGGACACCAACGCCCAAGACCCAGAAGAGCAAAACGAAAGCTCA | |
| | | GGTTTCAGAGTCCAGCAGCGACTCCCGTGGGTCCACTCCAGCCAA | |
| | | GAGACGCAAAGCTCCCAAGAAGAGACGGAGGCGCAGGGGTCGGT | |
| | | ACAAGACCAACTACTCCTCCAGCTCCGAGAGCAGCGAGTTCTCCG | |
| | | ACTCCAGCTCCAGCAACTCGCAACCCAAGTCCTCAAAGTCCAAGC | |
| | | AGGGCACAGCCTACACCCCCTATTATCTTCCCAAGCATAA | |
| CAF05737.1 | AJ620223.1 | ATGCGTTTTCGCAGGGTTGCCCAGAAAAGGAAAGTGCTTTTGCAA | 131 |
| | | ACTGTGCCAGCTGCAAAGAAGGCTAGGCGGCTTCTAGGTATGTGG | |
| | | CAGCCCCCCACGCACAATGTCCCGGGCATCGAGAGAAACTGGTA | |
| | | CGAGAGCTGTTTTAGATCCCACGCTGCTGTTTGTGGCTGTGGCGA | |
| | | TTTTGTTGGCCATCTTAATCATCTGGCAACTACTCTGGGTCGTCCT | |
| | | CCGCGTCCTGGGCCCCCAGGCGGACCCCGCACGCCGCAAATAAG | |
| | | AAACCTGCCAGCGCTCCCGGCGCCCCAGGGCGAGCCCGGTGACA | |
| | | GAGCGCCATGGCATGGGGCTTCTGGGGCCGACGCCGCCGGTGG | |
| | | AGACGATGGAGAGCGCGGCGCAGACGGTGGAGACCCCGCAGAC | |
| | | GTAGGAGACGACGCCCTACTCGCCGCTTTCGAGCTCGTCGAAGA | |
| | | GTAA | |
| CAF05738.1 | AJ620223.1 | ATGGCATGGGGCTTCTGGGGCCGACGCCGCCGGTGGAGACGATG | 132 |
| | | GAGAGCGCGGCGCAGACGGTGGAGACCCCGCAGACGTAGGAGA | |
| | | CGACGCCCTACTCGCCGCTTTCGAGCTCGTCGAAGAGTAAGGAG | |
| | | GCGCGGGGGGAGGTGGCGCAGACGCTACAGAAAATGGCGACGG | |
| | | GGCAGACGCAGACGGACTCATAGAAAAAAGATAGTCATAAAACAG | |
| | | TGGCAACCAAACTTTATAAGACGCTGCTACATCATAGGGTACTTAC | |
| | | CACTTATATTCTGCGGCGAAAATACAACCGCCCAGAACTTTGCCAC | |
| | | TCACTCGGACGACATGATAAGCAAAGGACCGTACGGGGGGGGCA | |
| | | TGACTACCACCAAATTCACTCTGAGAATACTGTACGACGAGTTTAC | |
| | | CAGGTTTATGAACTTTTGGACTGTCAGTAACGGAGACCTAGACCTG | |
| | | TGTAGATACGTGGGCTGCAAACTAATATTTTTTAAACACCCCACGG | |
| | | TGGACTTTATAGTACAGATAAACACTCAGCCTCCTTTCTTAGACAC | |
| | | GCACCTCACCGCGGCCAGCATACACCCGGGCATCATGATGCTCA | |
| | | GCAAGAGACACATACTAATACCCTCTCTAAAGACCCGGCCCAGCA | |
| | | GAAAACACAGGGTGGTCGTCAGGGTGGGCGCCCCAAGACTTTTTC | |
| | | AGGACAAGTGGTACCCCCAGTCAGACCTGTGTGACACAGTTCTGC | |
| | | TTTCCATATTTGCAACCGCCTGCGACTTGCAATATCCGTTCGGCTC | |
| | | ACCACTAACTGACAACCCTTGCGTCAACTTCCAGATCCTGGGGCC | |
| | | CCAGTACAAAAAACACCTTAGTATTAGCTCCACTATGGATCAAACT | |
| | | AACGAAAACCATTATAAAGAAAACTTATTTAACAAAACTGAACTATA | |
| | | CAACACCTTTCAAACCATAGCTCAGCTTAAAGAGACAGGACACATT | |
| | | TCAGGCATTAGTCCTACTTGGAATGAAGTCCAGAATTCAACAACAC | |
| | | TTACTAAAGAAGGTGACAATGCCACTCAGAGTAGAGACACTTGGTA | |
| | | TAAAGGAAATACATACAACGGTAAGATATGCCAGTTAGCACAAATA | |
| | | ACCAGAAACAGGTTTAAAAATGCAACCAAAGGAGCACTACCAAACT | |
| | | ACCCCACAATAATGTCCACAGACCTATATGAATACCACTCAGGCAT | |
| | | ATACTCCAGCATATGTCTATCAGCTGGCAGGAGCTACTTTGAAACC | |
| | | ACCGGGGCCTACTCTGACATTATATACAACCCTTTCACAGACAAAG | |
| | | GCACAGGCAACATAATCTGGATAGACTACCTCACAAAAGAAGACA | |
| | | CCATTTTTGTGAAAAACAAAAGCAAATGCGAGATAATGGACATGCC | |
| | | CCTGTGGGCGGCCTGCACAGGATACACAGAGTTTTGTGCAAAGTA | |
| | | TACAGGCGACTCTGCCATTATCTACAATGCAAGAATACTCATAAGA | |
| | | TGCCCATACACTGAGCCCATGTTAATAGACCACTCAGACCCAAACA | |
| | | AAGGCTTCGTTCCCTACTCATTTAACTTTGGCAACGGAAAGATGCC | |
| | | CGGAGGCAGCTCCAACGTGCCCATAAGAATGAGAGCCAAGTGGTA | |
| | | CGTGAACATATTCCACCAAAAAGAAGTATTAGAGAGCATAGTACAG | |
| | | TCCGGACCGTTTGGGTACAAGGGCGACATAAAATCAGCTGTACTA | |
| | | GCCATGAAATACAGATTTCACTGGAAGTGGGGCGGAAACCCTATA | |
| | | TCCAAACAGGTCGTCAGGAATCCCTGCTCCAACTCCAGCTCCTCC | |
| | | GCGGCCCATAGAGGACCTCGCAGCGTACAAGCGGTTGACCCGAA | |
| | | ATACAATACCCCAGAGGTCACGTGGCACTCGTGGGACATTAGACG | |
| | | AGGACTCTTTGGCAAAGCAGGTATTAAAAGAATGCAACAGGAATCA | |
| | | GATGCTCTTTACATTCCTCCAGGACCAATCAAGAGACCTCGCAGG | |
| | | GACACCAACGCCCAAGACCCAGAAGAGCAAAACGAAAGCTCAGGT | |
| | | TTCAGAGTCCAGCAGCGACTCCCGTGGGTCCACTCCAGCCAAGAG | |
| | | ACGCAAAGCTCCCAAGAAGAGACGGAGGCGCAGGGGTCGGTACA | |
| | | AGACCAACTACTCCTCCAGCTCCGAGAGCAGCGAGTTCTCCGACT | |
| | | CCAGCTCCAGCAACTCGCAACCCAAGTCCTCAAAGTCCAAGCAGG | |
| | | GCACAGCCTACACCCCCTATTATCTTCCCAAGCATAA | |
| CAF05778.1 | AJ620224.1 | ATGCGTTTTCGCAGGGTTGCCCAGAAAAGGAAAGTGCTTTTGCAA | 133 |
| | | ACTGTGCCAGCTGCAAAGAAGGCTAGGCGGCTTCTAGGTATGTGG | |
| | | CAGCCCCCCACGCACAATGTCCCGGGCATCGAGAGAAACTGGTA | |
| | | CGAGAGCTGTTTTAGATCCCACGCTGCTGTTTGTGGCTGTGGCGA | |
| | | TTTTGTTGGCCATCTTAATCATCTGGCAACTACTCTGGGTCGTCCT | |
| | | CCGCGTCCTGGGCCCCCAGGCGGACCCCGCACGCCGCAAATAAG | |
| | | AAACCTGCCAGCGCTCCCGGCGCCCCAGGGCGAGCCCGGTGACA | |
| | | GAGCGCCATGGCATGGGGCTTCTGGGGCCGACGCCGCCGGTGG | |
| | | AGACGATGGAGAGCGCGGCGCAGACGGTGGAGATCCCGCAGAC | |
| | | GTAGGAGACGACGCCCTACTCGCCGCTTTCGAGCTCGTCGAAGA | |
| | | GTAA | |
| CAF05779.1 | AJ620224.1 | ATGGCATGGGGCTTCTGGGGCCGACGCCGCCGGTGGAGACGATG | 134 |
| | | GAGAGCGCGGCGCAGACGGTGGAGATCCCGCAGACGTAGGAGA | |
| | | CGACGCCCTACTCGCCGCTTTCGAGCTCGTCGAAGAGTAAGGAG | |
| | | GCGCGGGGGGAGGTGGCGCAGACGCTACAGAAAATGGCGACGG | |
| | | GGCAGACGCAGACGGACTCATAGAAAAAAGATAGTCATAAAACAG | |
| | | TGGCAACCAAACTTTATAAGACGCTGCTACATCATAGGGTACTTAC | |
| | | CACTTATATTCTGCGGCGAAAATACAACCGCCCAGAACTTTGCCAC | |
| | | TCACTCGGACGACATGATAAGCAAAGGACCGTACGGGGGGGGCA | |
| | | TGACTACCACCAAATTCACTCTGAGAATACTGTACGACGAGTTTAC | |
| | | CAGGTTTATGAACTTTTGGACTGTCAGTAACGAAGACCTAGACCTG | |
| | | TGTAGATACGTGGGCTGCAAACTAATATTTTTTAAACACCCCACGG | |
| | | TGGACTTTATAGTACAGATAAACACTCAGCCTCCTTTCTTAGACAC | |
| | | GCACCTCACCGCGGCCAGCATACACCCGGGCATCATGATGCTCA | |
| | | GCAAGAGACACATACTAATACCCTCTCTAAAGACCCGGCCCAGCA | |
| | | GAAAGCACAGGGTGGTCGTCAGGGTGGGCGCCCCAAGACTTTTT | |
| | | CAGGACAAGTGGTACCCCCAGTCAGACCTGTGTGACACAGTTCTG | |
| | | CTTTCCATATTTGCAACCGCCTGCGACTTGCAATATCCGTTCGGCT | |
| | | CACCACTAACTGACAACCCTTGCGTCAACTTCCAGATCCTGGGGC | |
| | | CCCAGTACAAAAAACACCTTAGTATTAGCTCCACTATGGATCAAAC | |
| | | TAACGAAAACCATTATAAAGAAAACTTATTTAACAAAACTGAACTAT | |
| | | ACAACACCTTTCAAACCATAGCTCAGCTTAAAGAGACAGGACACAT | |
| | | TTCAGGCATTAGTCCTACTTGGAATGAAGTCCAGAATTCAACAACA | |
| | | CTTACTAAAGGAGGTGACAATGCCACTCAGAGTAGAGACACTTGG | |
| | | TATAAAGGAAATACATACAACGAGAACATATGCAAGTTAGCAGAGG | |
| | | TAACCAGAAACAGATTTAAAAATGCAACCAAAGGAGCACTACCAAA | |
| | | CTACCCCACAATAATGTCCACAGACCTATATGAATACCACTCAGGC | |
| | | ATATACTCCAGCATATATCTATCAGCGGGCAGGAGCTACTTTGAAA | |
| | | CCACCGGGGCCTACTCTGACATTATATACAACCCTTTCACAGACAA | |
| | | AGGCACAGGCAACATAATCTGGATAGACTACCTCACAAAAGAAGA | |
| | | CACCATTTTTGTGAAAAACAAAAGCAAATGCGAAATAATGGACATG | |
| | | CCCCTGTGGGCGGCCTGCACGGGATACACAGAGTTTTGTGCAAAG | |
| | | TATACAGGCGACTCTGCCATTATCTACAATGCAAGAATACTCATAA | |
| | | GATGCCCATACACTGAGCCCATGTTAATAGACCACTCAGACCCAAA | |
| | | CAAAGGCTTCGTTCCCTACTCATTTAACTTTGGCAACGGAAAGATG | |
| | | CCCGGAGGCAGCTCCAACGTGCCCATAAGAATGAGAGCCAAGTG | |
| | | GTACGTGAACATATTCCACCAAAAAGAAGTATTAGAGAGCATAGTA | |
| | | CAGTCCGGACCGTTTGGGTACAAGGGCGACATAAAATCAGCTGTA | |
| | | CTAGCCATGAAATACAGATTTCACTGGAAGTGGGGCGGAAACCCT | |
| | | ATATCCAAACAGGTCGTCAGGAATCCCTGCTCCAACTCCAGCCCC | |
| | | TCCGCGGCCCATAGAGGACCTCGCAGCGTACAAGCGGTTGACCC | |
| | | GAAATACAATACCCCAGAGGTCACGTGGCACTCGTGGGACATTAG | |
| | | ACGAGGACTCTTTGGCAAAGCAGGTATTAAAAGAATGCAACAGGA | |
| | | ATCAGATGCTCTTTACATTCCTCCAGGACCAATCAAGAGACCTCGC | |
| | | AGGGACACCAACGCCCAAGACCCAGAAGAGCAAAACGAAAGCTC | |
| | | AGGTTTCAGGGTCCAGCAGCGACTCCCGTGGGTCCACTCCAGCC | |
| | | AAGAGACGCAAAGCTCCCAAGAAGAGACGGAGGCGCAGGGGTCG | |
| | | GTACAAGACCAACTACTCCTCCAGCTCCGAGAGCAGCGAGTTCTC | |
| | | CGACTCCAGCTCCAGCAACTCGCAACCCAAGTCCTCAAAGTCCAA | |
| | | GCAGGGCACAGCCTACACCCCCTATTATCTTCCCAAGCATAA | |
| CAF05739.1 | AJ620225.1 | ATGCGTTTTCGCAGGGTTGCCCAGAAAAGGAAAGTGCTTTTGCAA | 135 |
| | | ACTGTGCCAGCTGCAAAGAAGGCTAGGCGGCTTCTAGGTATGTGG | |
| | | CAGCCCCCCACGCACAATGTCCCGGGCATCGAGAGAAACTGGTA | |
| | | CGAGAGCTGTTTTAGATCCCACGCTGCTGTTTGTGGCTGTGGCGA | |
| | | TTTTGTTGGCCATCTTAATCATCTGGCAACTACTCTGGGTCGTCCT | |
| | | CCGCGTCCTGGGCCCCCAGGCGGACCCCGCACGCCGCAAATAAG | |
| | | AAACCTGCCAGCGCTCCCGGCGCCCCAGGGCGAGCCCGGTGACA | |
| | | GAGCGTCATGGCGTGGGGCTTCTGGGGCCGACGCCGCCGGTGG | |
| | | AGACGATGGAGAGCGCGGCGCAGACGGTGGGGACCCCGCAGAC | |
| | | GTAGGAGACGACGCCCTCCTC | |
| CAF05740.1 | AJ620225.1 | ATGGCGTGGGGCTTCTGGGGCCGACGCCGCCGGTGGAGACGAT | 136 |
| | | GGAGAGCGCGGCGCAGACGGTGGGGACCCCGCAGACGTAGGAG | |
| | | ACGACGCCCTCCTCGCCGCTTTCGAGCTCGTCGAAGAGTAAGGAG | |
| | | GCGCGGGGGGAGGTGGCGCAGACGCTACAGAAAATGGCGACGG | |
| | | GGCAGACGCAGACGGACTCACAGAAAAAAGATAGTCATAAAACAG | |
| | | TGGCAACCAAACTTTATAAGACGCTGCTACATCATAGGGTACTTAC | |
| | | CACTTATATTCTGCGGCGAAAATACAACCGCCCAGAACTTTGCCAC | |
| | | TCACTCGGACGACATGATAAGCAAAGGACCGTACGGGGGGGGCA | |
| | | TGACTACCACCAAATTCACTCTGAGAATACTGTACGACGAGTTTAC | |
| | | CAGGTTTATGAACTTTTGGACTGTCAGTAACGAAGACCTAGACCTG | |
| | | TGTAGATACGTGGGCTGCAAACTAATATTTTTTAAACACCCCACGG | |
| | | TGGACTTTATAGTACAGATAAACACTCAGCCTCCTTTCTTAGACAC | |
| | | GCACCTCACCGCGGCCAGCATACACCCGGGCATCATGATGCTCA | |
| | | GCAAGAGACACATACTAATACCCTCTCTAAAGACCCGGCCCAGCA | |
| | | GAAAACACAGGGTGGTCGTCAGGGTGGGCGCCCCAAGACTTTTTC | |
| | | AGGACAAGTGGTACCCCCAGTCAGACCTGTGTGACACAGTTCTGC | |
| | | TTTCCATATTCGCAACCGCCTGCGACTTGCAATATCCGTTCGGCTC | |
| | | ACCACTAACTGACAACCCTTGCGTCAACTTCCAGATCCTGGGGCC | |
| | | CCAGTACAAAAAACACCTTAGTATTAGCTCCACTATGGATGACACT | |
| | | AACAAAGCACATTATGAAGAAAACTTATTTAATAAAACTGAACTATA | |
| | | CAACACCTTTCAAACCATAGCTCAGCTTAGAGACACAGGACAAACT | |
| | | GCAAACGCTAGTCCTAATTGGAATGAGGTCCAGAATACAGCAGCA | |
| | | CTTCAGTTATCAGGTGCAAATGCCACTAGCAGCAAAGACACTTGGT | |
| | | ATAAAGGTAATACATACACGAAAGACATATCAAAGTTAGCAGAAAA | |
| | | AACCAGACAAAGATTTAAAGCTGCAACAATAGCAGCACTACCAAAC | |
| | | TACCCCACAATAATGTCCACAGACCTATATGAATACCACTCAGGCA | |
| | | TATACTCCAGCATATATTTATCAGCTGGCAGGAGCTACTTTGAAAC | |
| | | CACCGGGGCCTACTCTGACATTATATACAACCCTTTCACAGACAAA | |
| | | GGCACAGGCAACATAATCTGGATAGACTACCTCACAAAAGAAGAC | |
| | | ACCATTTTTGTAAAAAACAAAAGCAAATGCGAGATAATGGACATGC | |
| | | CCCTGTGGGCGGCCTGCACAGGATACACAGAGTTTTGTGCAAAGT | |
| | | ATACAGGCGACTCTGCCATTATCTACAATGCAAGAATACTCATAAG | |
| | | ATGCCCATACACTGAGCCCATGTTAATAGACCACTCAGACCCAAAC | |
| | | AAAGGCTTCGTTCCCTACTCATTTAACTTTGGCAACGGAAAGATGC | |
| | | CCGGAGGCAGCTCCAACGTACCGATAAGAATGAGAGCCAAATGGT | |
| | | ACGTGAACATATTCCACCAAAAGGAGGTTCTAGAGGCTATAGTACA | |
| | | AAGCGGACCGTTCGGGTACAAGGGCGACATAAAATCAGCTGTACT | |
| | | AGCCATGAAATACAGATTTCACTGGAAGTGGGGCGGAAACCCTAT | |
| | | ATCCAAACAGGTCGTCAGGAATCCCTGCTCCAACTCCAGCTCATC | |
| | | CGCGGCCCATAGAGGACCTCGCAGCGTACAAGCGGTTGACCCGA | |
| | | AATACAATACCTCAGAGGTCACGTGGCACTCGTGGGACATTAGAC | |
| | | GAGGACTCTTTGACAAAGCAGGTATTAAAAGAATGCAACAGGAATC | |
| | | AGATGCTCTTTACATTCCTCCAGGACCAATCAAGAGACCTCGCAG | |
| | | GGACACCAACGCCCAAGACCCAGAAGAGCAAAACGAAAGCTCAG | |
| | | GTTTCAGAGTCCAGCAGCGACTCCCGTGGGTCCACTCCAGCCAAG | |
| | | AGACGCAAAGCTTCCAAGAAGAGACGGAGGCGCAGGGGTCGGTA | |
| | | CAAGACCAACTACTCCTCCAGCTCCGAGAGCAGCGAGTTCTCCGA | |
| | | CTCCAGCACCAGCAACTCGCAACCCAAGTCCTCAAAGTCCAAGCA | |
| | | GGGCACAGCCTACACCCCCTATTATCTTCCCAAGCATAA | |
| CAF05741.1 | AJ620226.1 | ATGCGTTTTTCCAGGATTGCTCGCTCGAAAAGGAAAGTGCCACTG | 137 |
| | | CCAACACTGCCAATACCACCGCCGCCTGGGACTATGAGCTGGCG | |
| | | CCCTCCGGTCCACAATGCCGCTGGAATCGACCGTAACTGGTTCGA | |
| | | ATCCTGTTTCAGATCTCACGCTAGCAGTTGCGGCTGTGGAAATTTT | |
| | | ATTGGCCATCTTAATACTCTCGCTACTCGCTACGGCTTTACTCCTG | |
| | | GGCCCGCGCCGCCGCCTGGTGGTCCAGGCCCGCGGCCGCCAGT | |
| | | ACCAGTGAGGCCCCGGCACCTGGCCGGAGACGGTAACCAGCCCA | |
| | | GGGCCCTGCCATGGCGTGGGGATGGTGGAGACGCAGACGCTGG | |
| | | CCCACCTACAGAAGGTGGCGGCGCTGGAGACGCCGCAGGAGAGT | |
| | | ACCGCGACGAAGACCTCGAAGAGCTGTTCGCCGCTATGGAAAGA | |
| | | GACGAGTAA | |
| CAF05742.1 | AJ620226.1 | ATGGTGGAGACGCAGACGCTGGCCCACCTACAGAAGGTGGCGGC | 138 |
| | | GCTGGAGACGCCGCAGGAGAGTACCGCGACGAAGACCTCGAAGA | |
| | | GCTGTTCGCCGCTATGGAAAGAGACGAGTAAGGAGGCGCCGGTG | |
| | | GGGAGGCGGCGGTACCGAAGGGGCTACAGACGCAGGGTCGCGG | |
| | | TCAGACTGAGACGCAGACGCAGACGGGGACGTAAGAGACTTGTA | |
| | | CTTACTCAGTGGCAGCCCCAGACCCGTAGAAAGTGCACCATCACC | |
| | | GGGTACCTCCCGGTGGTATGGTGCGGCTACCTCCGGGCCGCCAA | |
| | | AAACTATGCCTACCACTCTGACGACTCCACAAAGCAGCCGGACCC | |
| | | CTTTGGGGGCGCGCTGAGCACTACCTCCTTTAACCTTAAGGTGCT | |
| | | GTACGACCAGCACCAGAGAGGACTCAACAGGTGGTCTTTCCCTAA | |
| | | CGACCAACTGGACCTAGCTCGCTACAGGGGGTGCACACTTACGTT | |
| | | CTACAGACAGAAAGCCACTGACTTTATAGCTATTTATGACATCTCC | |
| | | GCCCCATACAAACTAGACAAGTACAGCTCTCCCAGCTATCACCCC | |
| | | GGCAACATGATAATGCAGAAAAAGAAAATTCTCATTCCCAGCTACG | |
| | | ACACTAACCCCAGGGGCCGCCAAAAAATAGTAGTTAAAATCCCCC | |
| | | CCCCTAAACTGTTCGTGGATAAGTGGTATGCACAGGAGGACCTGT | |
| | | GCGACGTTAATCTTGTGACACTTGCGGTCAGCGCAGCTTCCTTTAC | |
| | | ACATCCGTTCGGCTCACCACTAACGAACAACCCTTGTGTAACCTTC | |
| | | CAGGTACTTGACTCAATATACTATTCCGTAATAGGTTACGGTTCCT | |
| | | CAGATCAGAAAAAAAAACAAGTACTTGAAACTCTCTATAACGAAAA | |
| | | TGCATACTGGGCCTCACACTTAACTCCTTACTTTACCACTGGCCTT | |
| | | AAAATTCCATATCCAGATACTAAGAATCCCAGCACTACTGCATCTG | |
| | | TTACTCCAAACACGCTATTTACAACAGGTAGCTACGACTCAAACAT | |
| | | TAAAATAGCAGGAGACAGCAACTACAACTGGTACCCCTACAACCTT | |
| | | AAAAACAAAATAGACAAACTTCATAAAATTAGAGAACAATACTTTAA | |
| | | ATGGGAAACAGATGAAGGCCCCCAAGCCACATCTGATTATGGCAA | |
| | | ACACCACACTTGGACTAAACCCACCGATGACTACTACGAATACCAC | |
| | | CTAGGTTTATTTAGTCCCATATTCATAGGACCCACCAGAAGCAACA | |
| | | AACTATTTGCAACCGCCTACCAGGACGTTACTTACAACCCCCTAAA | |
| | | CGACAAGGCGGTGGGAAACAAGTTCTGGTTTCAGTACAACACAAA | |
| | | AGCAGACACCCAGGTGGCCAAACAAGGCTGCTACTGCATGCTAGA | |
| | | AGACATTCCCCTCTGGGCCGCCATGTATGGCTACTCTGACTTTATA | |
| | | GAGACCGAGCTAGGCCCCTTCCAAGACGCAGAGACGGTGGGCTA | |
| | | TATCTGTGTAATATGCCCCTACACCGAGCCCCCCATGTACAACAAA | |
| | | CACAATCCCATGCAGGGTTACGTGTTTTATGACTCGTTTTTTGGCA | |
| | | ATGGCAAGTGGATAGACGGACGGGGACACATAGAGCCTTACTGG | |
| | | CTCTGCCGCTGGAGGCCAGAAATGCTTTTCCAGCAGCAGGTTATG | |
| | | AGAGACATTGTGCAGACCGGGCCCTGGAGCTATAAAGACGAAAGC | |
| | | AAAAACTGTGTTCTGCCCATGAAGTATAAGTTCAGATTCACATGGG | |
| | | GCGGCAATATGGTCTCCCAACAGACAATCAGAAACCCCTGCAAGA | |
| | | CTGACGGACAACTTGCCCCCTCCGGTAGACAGCCTAGAGAAGTAC | |
| | | AAGTTGTTGACCCACTCACCATGGGTCCCCGCTGGGTTTTCCACT | |
| | | CCTGGGACTGGAGACGTGGCTACCTTAGTGAGACAGCTCTCAGAC | |
| | | GCCTGCGAGAAAAACCACTCGACTATGAGGCGTATATGCAAAAAC | |
| | | CAAAAAGACCTAGACTGTTCCCTGTTACAGAGGGCGACGACCAGT | |
| | | CCCCGCAGCAAGGCGACGACTGGTGTTCAGAGGAAGAAAAGTCG | |
| | | CCGCAGTTTACCGAAGAGACGACGCAGACGCTACAGCTCCAGCTC | |
| | | CAGCGCCAGCTCCGGCGACAGCAGCGACTCGGAGAGCAGCTCCA | |
| | | ACTCCTACAACACCACCTCCTCAAAACGCAAGCGGGCCTCCAAAT | |
| | | AAACCCATTATTATTGGTCCGGCAGTAA | |
| CAF05743.1 | AJ620227.1 | ATGCACTTTTCTCGAATAAGCAGAAAGAAAGGGAAAGTGCTACTGC | 139 |
| | | TTTGCGTGCCAGCAGTTAAGAAAAAACCAACTGCTATGAGCTTCTG | |
| | | GAGACCTCCGATGCACAATGTCACGGGGATCCAACGCCTGTGGTA | |
| | | CGAGTCCTTTCACCGTGGCCATGCTGCTTTTTGTGGTTGTGGGGA | |
| | | TCCTGTACTTCACATTACTGCACTTGCTGAGACATATGGCCATCCA | |
| | | ACAGGCCCGAGACCTTCTGGGTCATCGGGAATAGATCCCACTCCG | |
| | | CCCATCCGTAGAGCCAGGCCTGCCCCGGCCGCTCCGGAACCCCC | |
| | | ACAGGTTGACTCCAGACCGGCCCTGCCATGGCATGGAGATGGTG | |
| | | GAAGCGACGGAGGCGCTGGTGGCTCCGCAAGCGGTGGACCCGT | |
| | | GGCAGACTTCGCAGACGATGGCCTAGACCAGCTCGTCGCCGCCC | |
| | | TAGACGACGAAGAGTAA | |
| CAF05744.1 | AJ620227.1 | ATGGCATGGAGATGGTGGAAGCGACGGAGGCGCTGGTGGCTCCG | 140 |
| | | CAAGCGGTGGACCCGTGGCAGACTTCGCAGACGATGGCCTAGAC | |
| | | CAGCTCGTCGCCGCCCTAGACGACGAAGAGTAAGGAGACGCAGA | |
| | | CGGTGGAGGAGGGGGCGACCCAGACGCAGGCTGTACCGACGCT | |
| | | ACAGACGCAAAAAACGTAGGAGACGAAAGCCCAAAATAATCTTAAA | |
| | | ACAATGGCAGCCAGACATTGTAAAGAGGTGCTACATAGTGGGCTA | |
| | | CATTCCTGCCATAATATGTGGGGCGGGCACCTGGTCTCACAACTA | |
| | | CACCAGCCACCTCCTAGACATTATCCCCAAAGGACCCTTTGGAGG | |
| | | AGGGCACAGCACTATGAGGTTCTCCCTAAAAGTACTCTTTGAAGAA | |
| | | CACCTCAGACACTTAAACTTTTGGACAAAAAGCAACCAGGACCTAG | |
| | | AACTCATAAGATACTTTAGATGCTCCTTTAAATTCTATAGAGACCAA | |
| | | GACACAGACTACATAGTACACTACAGCAGAAAAACTCCCCTGGGA | |
| | | GGAAACAGACTAACAGCGCCTAGCCTACACCCCGGTGTACAGATG | |
| | | CTTAGCAAAAACAAAATATTAGTACCTAGCTATGCTACAAAACCCAA | |
| | | GGGTGGGAGCTATGTAAAAGTAACCATAGCACCCCCCACACTACT | |
| | | AACTGACAAGTGGTACTTTAGCAAAGACATTTGTGACACAACCTTG | |
| | | GTTAACTTAGACGTTGTACTCTGCAACTTGCGGTTTCCGTTCTGCT | |
| | | CACCACAAACTGACAACCCTTGCATCACATTCCAAGTTCTGCATTC | |
| | | CTTGTACAACGACTTCCTCTCCATAGTAGATACTGAAAATTACAAAA | |
| | | CCACTTTTGTTACTACACTGACAACAAAATTAGGTACAACATGGGG | |
| | | TTCAAGACTAAATACATTTAGAACAGAAGGCTGCTACTCACACCCT | |
| | | AAACTACCTAAAAAACAACTAATTGCTGCAAATGACACAACATACTT | |
| | | TACATCACCTGATGGGCTCTGGGGAGACGCAGTTTTCGACATCTC | |
| | | AAAACCTCAAGTAATTACCGAAAATATGGAGTCTTACGCTAACTCA | |
| | | GCCAAACAAAGAGGGGTGAACGGAGACCCCGCTTTTTGCCACCTA | |
| | | ACAGGAATATACTCACCTCCCTGGCTAACACCAGGCAGAATATCC | |
| | | CCTGAAACCCCAGGACTTTACACAGACGTGACTTACAACCCATAC | |
| | | GCTGACAAAGGAGTAGGCAACAGAATATGGGTCGACTACTGCAGT | |
| | | AAAAAAGGCAACAAATATGACAATACAAGTAAATGCCTTTTAGAAG | |
| | | ACATGCCACTATGGATGGTATGCTTTGGATACGTAGACTGGGTAAA | |
| | | AAAAGAGACTGGCAACTGGGGTATTCCACTATGGGCTAGAGTACT | |
| | | TATCAGAAGCCCATACGCTGTTCCAAAACTGTATAATGAAGCAGAC | |
| | | CCAAACTATGGATGGGTACCTATTTCTTACTACTTTGGAGAAGGCA | |
| | | AAATGCCAAACGGAGACATGTACGTACCATTTAAAATAAGAATGAA | |
| | | ATGGTACCCTTCAATGTGGAACCAAGAGCCAGTGTTAAATGACTTA | |
| | | GCAAAGAGCGGACCGTTTGCATACAAAAACACAAAAACAAGCGTG | |
| | | ACTGTGACTGCCAAATATAAATTTACATTTAACTTCGGGGGCAACC | |
| | | CCGTACCCTCACAGATTGTACAAGATCCCTGCACACAGTCCACCTA | |
| | | CGACATCCCCGGCACCGGTAACCTGCCTCGCAGAACACAAGTCAT | |
| | | TGACCCGAAATTCCTCGGTCCCCACTATTCCTTCCACCGGTGGGA | |
| | | CTTCAGGCGTGGCCTCTTTGGCTCACAAGCTATTAAGAGAGTGTC | |
| | | AGAACAACCAACAACTTCTGAGTTTTTATTCTCAGGCCCAAAGAGA | |
| | | CCCAGAATCGATCAAGGTCCTTACATCCCGCCAGAAAAAGACTCA | |
| | | GGTTCACTCCAAAGAGAATCGAGACCGTGGAGCAGCTCGGAGAC | |
| | | CGAGGCAGAGACAGAAGCCCCCTCGGAAGAAGAGCCGGAGAACC | |
| | | AAGAAGAACAAGTACTCCAGTTGCAGCTCAGACAGCAGCTTCGAG | |
| | | AACAGCGAAAACTCAGACAGGGAATCCAGTGCCTATTCGAGCAAC | |
| | | TGATAACAACCCAACAGGGGGTCCACAAAAACCCATTGTTAGAGTA | |
| | | G | |
| CAF05745.1 | AJ620228.1 | ATGCACTTTTCTCGAATAAGCAGAAAGAAAAGGAAAGTGCTACTGC | 141 |
| | | TTTGCGTGCCAGCAGTTAAGAAAAAACCAACTGCTATGAGCTTCTG | |
| | | GAGACCTCCGATGCACAATGTCACGGGGATCCAACGCCTGTGGTA | |
| | | CGAGTCCCTTCACCGTGGCCATGCTGCTTTTTGTGGTTGTGGGGA | |
| | | TCCTGTACTTCACATTACTGCACTTGCTGAGACATATGGCCATCCA | |
| | | ACAGGCCCGAGACCTTCTGGGTCATCGGGAATAGATCCCACTCCG | |
| | | CCCATCCGTAGAGCCAGGCCTGCCCCGGCCGCTCCGGAACCCCC | |
| | | ACAGGTTGACTCCAGACCGGCCCTGCCATGGCATGGAGATGGTG | |
| | | GGAGCGACGGAGGCGCTGGTGGCTCCGCAAGCGGTGGACCCGT | |
| | | GGCAGACTTCGCAGACGATGGCCTAGACCAGCTCGTCGCCGCCC | |
| | | TAGACGACGAAGAGTAA | |
| CAF05746.1 | AJ620228.1 | ATGGCATGGAGATGGTGGGAGCGACGGAGGCGCTGGTGGCTCCG | 142 |
| | | CAAGCGGTGGACCCGTGGCAGACTTCGCAGACGATGGCCTAGAC | |
| | | CAGCTCGTCGCCGCCCTAGACGACGAAGAGTAAGGAGACGCAGA | |
| | | CGGTGGAGGAGGGGGCGACCCAGACGCAGACTGTACCGACGCTA | |
| | | CAGACGCAAAAAACGTAGGAGACGAAAGCCCAAAATAATCTTAAAA | |
| | | CAATGGCAGCCAGACATTGTAAAGAGGTGCTACATAGTGGGCTAC | |
| | | ATTCCTGCCATAATATGTGGGGCGGGCACCTGGTCTCACAACTAC | |
| | | ACCAGCCACCTCCTAGACATTATCCCCAAAGGACCCTTTGGAGGA | |
| | | GGGCACAGCACTATGAGGTTCTCCCTAAAAGTACTCTTTGAAGAAC | |
| | | ACCTCAGACACTTAAACTTTTGGACAAAAAGCAACCAGGACCTAGA | |
| | | ACTCATAAGATACTTTAGATGCTCCTTTAAATTCTATAGAGACCAAG | |
| | | ACACAGACTACATAGTACACTACAGCAGAAAAACTCCCCTGGGAG | |
| | | GAAACAGACTAACAGCGCCTAGCCTACACCCCGGTGTACAGATGC | |
| | | TTAGCAAAAACAAAATATTAGTACCTAGCTATGCTACAAAACCCAA | |
| | | GGGTGGGAGCTATGTAAAAGTAACCATAGCACCCCCCACACTACT | |
| | | AACTGACAAGTGGTACTTTAGCAAAGACATTTGTGACACAACCTTG | |
| | | GTTAACTTAGACGTTGTACTCTGCAACTTGCGGTTTCCGTTCTGCT | |
| | | CACCACAAACTGACAACCCTTGCATCACATTCCAAGTTCTGCATTC | |
| | | CTTGTACAACGACTTCCTCTCTATAGTAGATACTGAAAATTACAAAA | |
| | | CCACTTTTGTTACTACACTGACAACAAAATTAGGTACAACATGGGG | |
| | | TTCAAGACTAAATACATTTAGAACAGAAGGCTGCTACTCACACCCT | |
| | | AAACTACCTAAAAAACAACTAATTGCTGCAAATGACACAACATACTT | |
| | | TACATCACCTGATGGGCTCTGGGGAGACGCAGTTTTCGACATCTC | |
| | | AAAACCTCAAGTAATTACCGAAAATATGGAGTCTTACGCTAACTCA | |
| | | GCCAAACAAAGAGGGGTGAACGGAGACCCCGCTTTTTGCCACCTA | |
| | | ACAGGAATATACTCACCTCCCTGGCTAACACCAGGCAGAATATCC | |
| | | CCTGAAACCCCAGGACTTTACACAGACGTGACTTACAACCCATAC | |
| | | GCTGACAAAGGAGTAGGCAACAGAATATGGGTCGACTACTGCAGT | |
| | | AAAAAAGGCAACAAATATGACAATACAAGTAAATGCCTTTTAGAAG | |
| | | ACATGCCACTATGGATGGTATGCTTTGGATACGTAGACTGGGTAAA | |
| | | AAAAGAGACTGGCAANTGGGGTATTCCACTATGGGCTAGAGTACT | |
| | | TATCAGAAGCCCATACACTGTTCCAAAACTGTATAATGAAGCAGAC | |
| | | CCAAACTATGGATGGGTACCTATTTCTTACTACTTTGGAGAAGGCA | |
| | | AAATGCCAAACGGAGACATGTACGTACCATTTAAAATGAGAATGAA | |
| | | ATGGCACCCTTCAATGTGGAACCAAGAGCCAGTGTTAAATGACTTA | |
| | | GCAAAGAGCGGACCGTTTGCATACAAAAACACAAAAACAAGCGTG | |
| | | ACTGTGACTGCCAAATATAAATTTACATTTAACTTCGGGGGCAACC | |
| | | CCGTACCCTCACAGATTGTACAAGGTCCCTGCACACAGTCCACCT | |
| | | ACGACATCCCCGGCACCGGTAACCTGCCTCGCAGAATACAGGTCA | |
| | | TTGACCCGAAATTCCTCGGTCCCCACTATTCCTTCCACCGGTGGG | |
| | | ACTTCAGGCGTGGCCTCTTTGGCTCACAAGCTATTAAGAGAGTGT | |
| | | CAGAACAACCAACAACTTCTGAGTTTTTATTCTCAGGCCCAAAGAG | |
| | | ACCCAGAATCGATCAAGGTCCTTACATCCCGCCAGAAAAAGACTC | |
| | | AGGTTCACTCCAAAGAGAATCGAGACCGTGGAGCAGCTCGGAGAC | |
| | | CGAGGCAGAGACAGAAGCCCCCTCGGAAGAAGAGCCGGAGAACC | |
| | | AAGAAGAACAAGTACTCCAGTTGCAGCTCAGACAGCAGCTTCGAG | |
| | | AACAGCGAAAACTCAGACAGGGAATCCAGTGCCTATTCGAGCAAC | |
| | | TGATAACAACCCAACAGGGGGTCCACAAAAACCCATTGTTAGAGTA | |
| | | G | |
| CAF05747.1 | AJ620229.1 | ATGCACTTTTCTCGAATAAGCAGAAAGAAAAGGAAAGTGCTACTGC | 143 |
| | | TTTGCGTGCCAGCAGTTAAGAAAAAACCAACTGCTATGAGCTTCTG | |
| | | GAGACCTCCGATGCACAATGTCACGGGGATCCAACGCCTGTGGTA | |
| | | CGAGTCCCTTCACCGTGGCCATGCTGCTTTTTGTGGTTGTGGGGA | |
| | | TCCTGTACTTCACATTACCGCACTTGCTGAGACATATGGCCATCCA | |
| | | ACAGGCCCGAGACCTTCTGGGTCATCGGGAATAGATCCCACTCCG | |
| | | CCCATCCGTAGAGCCAGGCCTGCCCCGGCCGCTCCGGAACCCCC | |
| | | ACAGGTTGACTCCAGACCGGCCCTGCCATGGCATGGAGATGGTG | |
| | | GAAGCGACGGAGGCGCTGGTGGCTCCGCAAGCGGTGGACCCGT | |
| | | GGCAGACTTCGCAGACGATGGCCTAGACCAGCTCGTCGCCGCCC | |
| | | TAGACGACGAAGAGTAA | |
| CAF05748.1 | AJ620229.1 | ATGGCATGGAGATGGTGGAAGCGACGGAGGCGCTGGTGGCTCCG | 144 |
| | | CAAGCGGTGGACCCGTGGCAGACTTCGCAGACGATGGCCTAGAC | |
| | | CAGCTCGTCGCCGCCCTAGACGACGAAGAGTAAGGAGACGCAGA | |
| | | CGGTGGAGGAGGGGGCGACCCAGACGCAGACTGTACCGACGCTA | |
| | | CAGACGCAAAAAACGTAGGAGACGAAAGCCCAAAATAATCTTAAAA | |
| | | CAATGGCAGCCAGACATTGTAAAGAGGTGCTACATAGTGGGCTAC | |
| | | ATTCCTGCCATAATATGTGGGGCGGGCACCTGGTCTCACAACTAC | |
| | | ACCAGCCACCTCCTAGACATTATCCCCAAAGGACTCTTTGGAGGA | |
| | | GGGCACAGCACTATGAGGTTCTCCCTAAAAGTACTCTTTGAAGAAC | |
| | | ACCTCAGACACTTAAACTTTTGGACAAAAAGCAACCAGGACCTAGA | |
| | | ACTCATAAGATACTTTAGATGCTCCTTTAAATTCTATAGAGACCAAG | |
| | | ACACAGACTACATAGTACACTACAGCAGAAAAACTCCCCTGGGAG | |
| | | GAAACAGACTAACAGCGCCTAGCCTACACCCCGGTGTACAGTTGC | |
| | | TTAGCAAAAACAAAATATTAGTACCTAGCTATGCTACAAAACCCAA | |
| | | GGGTGGGAGCTATGTAAAAGTAACCATAGCACCCCCCACACTACT | |
| | | AACTGACAAGTGGTACTTTAGCAAAGACATTTGTGACACAACCTTG | |
| | | GTTAACTTAGACGTTGTACTCTGCAACTTGCGGTTTCCGTTCTGCT | |
| | | CACCACAAACTGACAACCCTTGCATCACATTCCAAGTTCTGCATTC | |
| | | CTTGTACAACGACTTCCTCTCTATAGTAGATACTGAAAATTACAAAA | |
| | | CCACTTTTGTTACTACACTGACAACAAAATTAGGTACAACATGGGG | |
| | | TTCAAGACTAAATACATTTAGAACAGAAGGCTGCTACTCACACCCT | |
| | | AAACTACCTAAAAAACAACTAATTGCTGCAAATGACACAACATACTT | |
| | | TACATCACCTGATGGGCTCTGGGGAGACGCAGTTTTCAACATCTC | |
| | | AAAACCTCAAGTAATTACCGAAAATATGGAGTCTTACGCTAACTCA | |
| | | GCCAAACAAAGAGGGGTGAACGGAGACCCCGCTTTTTGCCACCTA | |
| | | ACAGGAATATACTCACCTCCCTGGCTAACACCAGGCAGAATATCC | |
| | | CCTGAAACCCCAGGACTTTACACAGACGTGACTTACAACCCATAC | |
| | | GCTGACAAAGGAGTAGGCAACAGAATATGGGTCGACTACTGCAGT | |
| | | AAAAAAGGCAACAAATATGACAATACAAGTAAATGCCTTTTAGAAG | |
| | | ACATGCCACTATGGATGGTATGCTTTGGATACGTAGACTGGGTAAA | |
| | | AAAAGAGACTGGCAACTGGGGTATTCCACTATGGGCTAGAGTACT | |
| | | TATCAGAAGCCCATACACTGTTCCAAAACTGTATAATGAAGCAGAC | |
| | | CCAAACTATGGATGGGTACCTATTTCTTACTACTTTGGAGAAGGCA | |
| | | AAATGCCAAACGGAGACATGTACGTACCATTTAAAATAAGAATGAA | |
| | | ATGGCACCCTTCAATGTGGAACCAAGAGCCAGTGTTAAATGACTTA | |
| | | GCAAAGAGCGGACCGTTTGCATACAAAAACACAAAAACAAGCGTG | |
| | | ACTGTGACTGCCAAATATAAATTTACATTTAACTTCGGGGGCAACC | |
| | | CCGTACCCTCACAGATTGTACAAGATCCCTGCACACAGTCCACCTA | |
| | | CGACATCCCCGGCACCGGTAACCTGCCTCGCAGAATACAAGTCAT | |
| | | TGACCCGAAATTCCTCGGTCCCCACTATTCCTTCCACCGGTGGGA | |
| | | CTTCAGGCGTGGCCTCTTTGGCTCACAAGCTATTAAGAGAGTGTC | |
| | | AGAACAACCAACAACTTCTGAGTTTTTATTCTCAGGCCCAAAGAGA | |
| | | CCCAGAATCGATCAAGGTCCTTACATCCCGCCAGAAAAAGACTCA | |
| | | GGTTCACTCCAAAGAGAATCGAGACCGTGGAGCAGCTCGGAGAC | |
| | | CGAGGCAGAGACAGAAGCCCCCTCGGAAGAAGAGCCGGAGAACC | |
| | | AAGAAGAACAAGTACTCCAGTTGCAGCTCAGACAGCAGCTTCGAG | |
| | | AACAGCGAAAACTCAGACAGGGAATCCAGTGCCTATTCGAGCAAC | |
| | | TGATAACAACCCAACAGGGGGTCCACAAAAACCCATTGTTAGAGTA | |
| | | G | |
| CAF05780.1 | AJ620230.1 | ATGCACTTTTCTCGAATAAGCAGAAAGAAAAGGAAAGTGCTACTGC | 145 |
| | | TTTGCGTGCCAGCAGCTAAGAAAAAACCAACTGCTATGAGCTTCTG | |
| | | GAAACCTCCGGTACACAATGTCACGGGGATCCAACGCATGTGGTA | |
| | | TGAGTCCTTTCACCGTGGCCACGCTTCTTTTTGTGGTTGTGGGAAT | |
| | | CCTATACTTCACATTACTGCACTTGCTGAAACATATGGCCATCCAA | |
| | | CAGGCCCGAGACCTTCTGGGCCACCGGGAGTAGACCCCAACCCC | |
| | | CACATCCGTAGAGCCAGGCCTGCCCCGGCCGCTCCGGGGCCCTC | |
| | | ACAGGTTGATTCGAGACCAGCCCTGACATGGCATGGGGATGGTG | |
| | | GAAGCGACGGAGGCGCTGGTGGTTCCGGAAGCGGTGGACCCGT | |
| | | GGCAGACTTCGCAGACGATGGCCTCGATCAGCTCGTCGCCGCCC | |
| | | TAGACGACGAAGAGTAA | |
| CAF05781.1 | AJ620230.1 | ATGGCATGGGGATGGTGGAAGCGACGGAGGCGCTGGTGGTTCCG | 146 |
| | | GAAGCGGTGGACCCGTGGCAGACTTCGCAGACGATGGCCTCGAT | |
| | | CAGCTCGTCGCCGCCCTAGACGACGAAGAGTAAGGAGGCGCAGA | |
| | | CGGTGGAGGAGGGGGAGACGAAAAACAGGGACTTACAGACGCAG | |
| | | GAGACGCTTTAGACGCAGGAGACGAAAAGCAAAACTTATAATAAAA | |
| | | CTGTGGCAACCTGCAGTAATTAAAAGATGCAGAATAAAGGGATACA | |
| | | TACCACTGATTATAAGTGGGAACGGTACCTTTGCCACAAACTTTAC | |
| | | CAGTCACATAAATGACAGAATAATGAAAGGCCCCTTCGGGGGAGG | |
| | | ACACAGCACTATGAGGTTCAGCCTCTACATTTTGTTTGAGGAGCAC | |
| | | CTCAGACACATGAACTTCTAG | |
| CAF05782.1 | AJ620230.1 | ATGGCAGTTGAGGCTGACTTGCGGTTTCCGTTCTGCTCACCACAA | 147 |
| | | ACTGACAACACTTGCATCAGCTTCCAGGTCCTTAGTTCCGTTTACA | |
| | | ACAACTACCTCAGTATTAATACCTTTAATAATGACAACTCAGACTCA | |
| | | AAGTTAAAAGAATTTTTAAATAAAGCATTTCCGACAACAGGCACAAA | |
| | | AGGAACAAGTTTAAATGCACTAAATACATTTAGAACAGAAGGATGC | |
| | | ATAAGTCACCCACAACTAAAAAAACCAAACCCACAAATAAACAAAC | |
| | | CATTAGAGTCACAATACTTTGCACCTTTAGATGCCCTCTGGGGAGA | |
| | | CCCCATATACTATAATGATCTAAATGAAAACAAAAGTTTGAACGATA | |
| | | TCATTGAGAAAATACTAATAAAAAACATGATTACATACCATGCAAAA | |
| | | CTAAGAGAATTTCCAAATTCATACCAAGGAAACAAGGCCTTTTGCC | |
| | | ACCTAACAGGCATATACAGCCCACCATACCTAAACCAAGGCAGAAT | |
| | | ATCTCCAGAAATATTTGGACTGTACACAGAAATAATTTACAACCCTT | |
| | | ACACAGACAAAGGAACTGGAAACAAAGTATGGATGGACCCACTAA | |
| | | CTAAAGAGAACAACATATATAAAGAAGGACAGAGCAAATGCCTACT | |
| | | GACTGACATGCCCCTATGGACTTTACTTTTTGGATATACAGACTGG | |
| | | TGTAAAAAGGACACTAATAACTGGGACTTACCACTAAACTACAGAC | |
| | | TAGTACTAATATGCCCTTATACCTTTCCAAAATTGTACAATGAAAAG | |
| | | GTAAAAGACTATGGGTACATCCCGTACTCCTACAAATTCGGAGCG | |
| | | GGTCAGATGCCAGACGGCAGCAACTACATACCCTTTCAGTTTAGA | |
| | | GCAAAGTGGTACCCCACAGTACTACACCAGCAACAGGTAATGGAG | |
| | | GACATAAGCAGGAGCGGGCCCTTTGCACCTAAGGTAGAAAAACCA | |
| | | AGCACTCAGCTGGTAATGAAGTACTGTTTTAACTTTAACTGGGGCG | |
| | | GTAACCCTATCATTGAACAGATTGTTAAAGACCCCAGCTTCCAGCC | |
| | | CACCTATGAAATACCCGGTACCGGTAACATCCCTAGAAGAATACAA | |
| | | GTCATCGACCCGCGGGTCCTGGGACCGCACTACTCGTTCCGGTC | |
| | | ATGGGACATGCGCAGACACACATTTAGCAGAGCAAGTATTAAGAG | |
| | | AGTGTCAGAACAACAAGAAACTTCTGACCTTGTATTCTCAGGCCCA | |
| | | AAAAAGCCTCGGGTCGACATCCCAAAACAAGAAACCCAAGAAGAA | |
| | | AGCTCACATTCACTCCAAAGAGAATCGAGACCGTGGGAGACCGAG | |
| | | GAAGAAAGCGAGACAGAAGCCCTCTCGCAAGAGAGCCAAGAGGT | |
| | | CCCCTTCCAACAGCAGTTGCAGCAGCAGTACCAAGAGCAGCTCAA | |
| | | GCTCAGACAGGGAATCAAAGTCCTCTTCGAGCAGCTCATAAGGAC | |
| | | CCAACAAGGGGTCCATGTAAACCCATGCCTACAGTAG | |
| CAF05749.1 | AJ620231.1 | ATGCACTTTTCTCGAATAAGCAGAAAGAAAAGGAAAGTGCTACTGC | 148 |
| | | TTTGCGTGCCAGCAGCTAAGAAAAAACCAACTGCTATGAGCTTCTG | |
| | | GAAACCTCCGGTACACAATGTCACGGGGATCCAACGCATGTGGTA | |
| | | TGAGTCCTTTCACCGTGGCCACGCTTCTTTTTGTGGTTGTGGGAAT | |
| | | CCTATACTTCACATTACTGCACTTGCTGAAACATATGGCCATCCAA | |
| | | CAGGCCCGAGACCTTCTGGGCCACCGGGAGTAGACCCCAACCCC | |
| | | CACATCCGTAGAGCCAGGCCTGCCCCGGCCGCTCCGGAGCCCTC | |
| | | ACAGGTTGATTCGAGACCAGCCCTGACATGGCATGGGGATGGTG | |
| | | GAAGCGACGGAGGCGCTGGTGGTTCCGGAAGCGGTGGACCCGT | |
| | | GGCAGACTTCGCAGACGATGGCCTCGATCAGCTCGTCGCCGCCC | |
| | | TAGACGACGAAGAGTAA | |
| CAF05750.1 | AJ620231.1 | ATGGCATGGGGATGGTGGAAGCGACGGAGGCGCTGGTGGTTCCG | 149 |
| | | GAAGCGGTGGACCCGTGGCAGACTTCGCAGACGATGGCCTCGAT | |
| | | CAGCTCGTCGCCGCCCTAGACGACGAAGAGTAAGGAGGCGCAGA | |
| | | CGGTGGAGGAGGGGGAGACGAAAAACAAGGACTTACAGACGCAG | |
| | | GAGACGCTTTAGACGCAGGGGACGAAAAGCAAAACTTATAATAAA | |
| | | ACTGTGGCAACCTGCAGTAATTAAAAGATGCAGAATAAAGGGATAC | |
| | | ATACCACTGATTATAAGTGGGAACGGTACCTTTGCCACAAACTTTA | |
| | | CCAGTCACATAAATGACAGAATAATGAAAGGCCCCTTCGGGGGAG | |
| | | GACACAGCACTATGAGGTTCAGCCTCTACATTTTGTTTGAGGAGCA | |
| | | CCTCAGACACATGAACTTCTGGACCAGAAGCAACGATAACCTAGA | |
| | | GCTAACCAGATACTTGGGGGCTTCAGTAAAAATATACAGGCACCC | |
| | | AGACCAAGACTTTATAGTAATATACAACAGAAGAACCCCTCTAGGA | |
| | | GGCAACATCTACACAGCACCCTCTCTACACCCAGGCAATGCCATTT | |
| | | TAGCAAAACACAAAATATTAGTACCAAGTTTACAGACAAGACCAAA | |
| | | GGGTAGAAAAGCAATTAGACTAAGAATAGCACCCCCCACACTCTTT | |
| | | ACAGACAAGTGGTACTTTCAAAAGGACATAGCCGACCTCACCCTTT | |
| | | TCAACATCATGGCAGTTGAGGCTGACTTGCGGTTTCCGTTCTGCTC | |
| | | ACCACAAACTGACAACACTTGCATCAGCTTCCAGGTCCTTAGTTCC | |
| | | GTTTACAACAACTACCTCAGTATTAATACCTTTAATAATGACAACTC | |
| | | AGACTCAAAGTTAAAAGAATTTTTAAATAAAGCATTTCCAACAACAG | |
| | | GCACAAAAGGAACAAGTTTAAATGCACTAAATACATTTAGAACAGA | |
| | | AGGATGCATAAGTCACCCACAACTAAAAAAACCAAACCCACAAATA | |
| | | AACAAACCATTAGAGTCACAATACTTTGCACCTTTAGATGCCCTCT | |
| | | GGGGAGACCCCATATACTATAATGATCTAAATGAAAACAAAAGTTT | |
| | | GAACGATATCATTGAGAAAATACTAATAAAAAACATGATTACATACC | |
| | | ATGCAAAACTAAGAGAATTTCCAAATTCATACCAAGGAAACAAGGC | |
| | | CTTTTGCCACCTAACAGGCATATACAGCCCACCATACCTAAACCAA | |
| | | GGCAGAATATCTCCAGAAATATTTGGACTGTACACAGAAATAATTT | |
| | | ACAACCCTTACACAGACAAAGGAACTGGAAACAAAGTATGGATGG | |
| | | ACCCACTAACTAAAGAGAACAACATATATAAAGAAGGACAGAGCAA | |
| | | ATGCCTACTGACTGACATGCCCCTATGGACTTTACTTTTTGGATAT | |
| | | ACAGACTGGTGTAAAAAGGACACTAATAACTGGGACTTACCACTAA | |
| | | ACTACAGACTAGTACTAATATGCCCTTATACCTTTCCAAAATTGTAC | |
| | | AATGAAAAAGTAAAAGACTATGGGTACATCCCGTACTCCTACAAAT | |
| | | TCGGAGCGGGTCAGATGCCAGACGGCAGCAACTACATACCCTTTC | |
| | | AGTTTAGAGCAAAGTGGTACCCCACAGTACTACACCAGCAACAGG | |
| | | TAATGGAGGACATAAGCAGGAGCGGGCCCTTTGCACCTAAGGTAG | |
| | | AAAAACCAAGCACTCAGCTGGTAATGAAGTACTGTTTTAACTTTAA | |
| | | CTGGGGCGGTAACCCTATCATTGAACAGATTGTTAAAGACCCCAG | |
| | | CTTCCAGCCCACCTATGAAATACCCGGTACCGGTAACATCCCTAG | |
| | | AAGAATACAAGTCATCGACCCGCGGGTCCTGGGACCGCACTACTC | |
| | | GTTCCGGTCATGGGACATGCGCAGACACACATTTAGCAGAGCAAG | |
| | | TATTAAGAGAGTGTCAGAACAACAAGAAACTTCTGACCTTGTATTC | |
| | | TCAGGCCCAAAAAAGCCTCGGGTCGACATCCCAAAACAAGAAACC | |
| | | CAAGAAGAAAGCTCACATTCACTCCAAAGAGAATCGAGACCGTGG | |
| | | GAGACCGAGGAAGAAAGCGAGACAGAAGCCCTCTCGCAAGAGAG | |
| | | CCAAGAGGTCCCCTTCCAACAGCAGTTGCAGCAGCAGTACCAAGA | |
| | | GCAGCTCAAGCTCAGACAGGGAATCAAAGTCCTCTTCGAGCAGCT | |
| | | CATAAGGACCCAACAAGGGGTCCATGTAAACCCATGCCTACGGTA | |
| | | G | |
| CAF05751.1 | AJ620232.1 | ATGCACTTTTCTCGAATAAGCAGAAAGAAAAGGAAAGTGCTACTGC | 150 |
| | | TTTGCGTGCCAGCAGCTAAGAAAAAACCAACTGCTATGAGCTTCTG | |
| | | GAAACCTCCGGTACACAATGTCACGGGGATCCAACGCATGTGGTA | |
| | | TGAGTCCTTTCACCGTGGCCACGCTTCTTTTTGTGGTTGTGGGAAT | |
| | | CCTATACTTCACATTACTGCACTTGCTGAAACATATGGCCATCCAA | |
| | | CAGGCCCGAGACCTTCTGGGCCACCGGGAGTAGACCCCAACCCC | |
| | | CACATCCGTAGAGCCAGGCCTGCCCCGGCCGCTCCGGAGCCCTC | |
| | | ACAGGTTGATTCGAGACCAGCCCTGACGTGGCATGGGGATGGTG | |
| | | GAAGCGACGGAGGCGCTGGTGGTTCCGGAAGCGGTGGACCCGT | |
| | | GGCAGACTTCGCAGACGATGGCCTCGATCAGCTCGTCGCCGCCC | |
| | | TAGACGACGAAGAGTAA | |
| CAF05752.1 | AJ620232.1 | ATGAAAGGCCCCTTCGGGGGAGGACACAGCACTATGAGGTTCAG | 151 |
| | | CCTCTACATTTTGTTTGAGGAGCGCCTCAGACACATGAACTTCTGG | |
| | | ACCAGAAGCAACGATAACCTAGAGCTAACCAGATACTTGGGGGCT | |
| | | TCAGTAAAAATATACAGGCACCCAGACCAAGACTTTATAGTAATAT | |
| | | ACAACAGAAGAACCCCTCTAGGAGGCAACATCTACACAGCACCCT | |
| | | CTCTACACCCAGGCAATGCCATTTTAGCAAAACACAAAATATTAGT | |
| | | ACCAAGTTTACAGACAAGACCAAAGGGTAGAAAAGCAATTAGACTA | |
| | | AGAATAGCACCCCCCACACTCTTTACAGACAAGTGGTACTTTCAAA | |
| | | AGGACATAGCCGACCTCACCCTTTTCAACATCATGGCAGTTGAGG | |
| | | CTGACTTGCGGTTTCCGTTCTGCTCACCACAAACTGGCAACACTTG | |
| | | CATCAGCTTCCAGGTCCTTAATTCCGTTTACAACAACTACCTCAGT | |
| | | ATTAATACCTTTAATAATGACAACTCAGACTCAAAGTTAAAAGAATT | |
| | | TTTAAATAAAGCATTTCCAACAACAGGCACAAAAGGAACAAGTTTA | |
| | | AATGCACTAAATACATTTAGAACAGAAGGATGCATAAGTCACCCAC | |
| | | AACTAAAAAAACCAAACCCACAAATAAACAAACCATTAGATTCACAA | |
| | | TACTTTGCACCTTTAGACGCCCTCTGGGGAGACCCCATATACTATA | |
| | | ATGATCTAAATGAAAAGAAAAGTTTGAAGGATATCATTGAGAACAT | |
| | | ACTAATAAAAAACATGATTACATACCATGAAAAACTAAGAGAGTTTC | |
| | | CAAATTCATACCAAGGAAACAAGGCCTTTTGCCACCTAACAGGCAT | |
| | | ATACAGCCCACCATACCTAAACCAAGGCAGAATATCTCCAGAAATA | |
| | | TTTGGACTGTACACAGAAATAATTTACAACCCTTACACAGACAAAG | |
| | | GAACTGGAAACAAAGTATGGATGGACCCACTAACTAAAGAGAACA | |
| | | ACATATATAAAGAAGGACAGAGCAAATGCCTACTGACTGACATGCC | |
| | | CCTATGGACTTTACTTTTTGGATATACAGACTGGTGTAAAAAGGAC | |
| | | ACTAATAACTGGGACTTACCACTAAACTACAGACTAGTACTAATAT | |
| | | GCCCTTATACCTTTCCAAAATTGTACAATGAAAAGGTAAAAGACTAT | |
| | | GGGTACATCCCGTACTCCTACAAATTCGGAGCGGGTCAGATGCCA | |
| | | GACGGCAGCAACTACATACCCTTTCAGTTTAGAGCAAAGTGGTAC | |
| | | CCCACAGTACTACACCAGCAACAGGTAATGGAGGACATAAGCAGG | |
| | | AGCGGGCCCTTTGCACCTAAGGTAGAAAAACCAGGCACTCAGCTG | |
| | | GTAATGAAGTACTGTTTTAACTTTAACTGGGGCGGTAACCCTATCA | |
| | | TTGAACAGATTGTTAAAGACCCCAGCTTCCAGCCCACCTATGAAAT | |
| | | ACCCGGTACCGGTGACATCCCTAGAAGAATACAAGTCATCGACCC | |
| | | GCGGGTCCTGGGACCGCACTACTCGTTCCGGTCATGGGACACGC | |
| | | GCAGACACACATTTAGCAGAGCAAGTATTAAGAGAGTGTCAGAAC | |
| | | AACAAGAAGCTTCTGACCTTGTATTCTCAGGCCCAAAAAAGCCTCG | |
| | | GGTCGACATCCCAAAACAAGAAACCCAAGAAGAAAGCTCACATTC | |
| | | ACTCCAAAGAGAATCGAGACCGTGGGAGACCGAGGAAGAAAGCG | |
| | | AGACAGAAGCCCTCTCGCAAGAGAGCCAAGAGGTCCCCTTCCAAC | |
| | | AGCAGTTGCAGCAGCAGTACCAAGAGCAGCTCAAGCTCAGACAGG | |
| | | GAATCAAAGTCCTCTTCGAGCAGCTCATAAGGACCCAACAAGGGG | |
| | | TCCATGTAAACCCATGCCTACAGTAG | |
| CAF05753.1 | AJ620233.1 | ATGCACTTTTCTCGAATAAGCAGAAAGAAAAGGAAAGTGCTACTGC | 152 |
| | | TTTGCGTGCCAGCAGCTAAGAAAAAACCAACTGCTATGAGCTTCTG | |
| | | GAAACCTCCGGTACACAATGTCACGGGGATCCAACGCATGTGGTA | |
| | | TGAGTCCTTTCACCGTGGCCACGCTTCTTTTTGTGGTTGTGGGAAT | |
| | | CCTATACTTCACATTACTGCACTTGCTGAAACATATGGCCGTCCAA | |
| | | CAGGCCCGAGACCTTCTGGGCCACCGGGAGTAGACCCCAACCCC | |
| | | CACATCCGTAGAGCCAGGCCTGCCCCGGCCGCTCCGGAGCCCTC | |
| | | ACAGGTTGATTCGAGACCAGCCCTGACATGGCATGGGGATGGTG | |
| | | GAAGCGACGGAGGCGCTGGTGGTTCCGGAAGCGGTGGACCCGT | |
| | | GGCAGACTTCGCAGACGATGGCCTCGATCAGCTCGTCGCCGCCC | |
| | | TAGACGACGAAGAGTAA | |
| CAF05754.1 | AJ620233.1 | ATGGCATGGGGATGGTGGAAGCGACGGAGGCGCTGGTGGTTCCG | 153 |
| | | GAAGCGGTGGACCCGTGGCAGACTTCGCAGACGATGGCCTCGAT | |
| | | CAGCTCGTCGCCGCCCTAGACGACGAAGAGTAAGGGGGCGCAGA | |
| | | CGGTGGAGGAGGGGGAGACGAAAAACAAGGACTTACAGACGCAG | |
| | | GAGACGCTTTAGACGCAGGAGACGAAAAGCAAAACTTATAGTAAA | |
| | | ACTGTGGCAACCTGCAGTAATTAAAAGATGCAGAATAAAGGGATAC | |
| | | ATACCACTGATTATAGGTGGGAACGGTACCTTTGCCACAAACTTTA | |
| | | CCAGTCACATAAATGACAGAATAATGAAAGGCCCCTTCGGGGGAG | |
| | | GACACAGCACTATGAGGTTCAGCCTCTACATTTTGTTTGAGGAGCA | |
| | | CCTCAGACACATGAACTTCTGGACCAGAAGCAACGATAACCTAGA | |
| | | GCTAACCAGATACTTGGGGGCTTCAGTAAAAATATACAGGCACCC | |
| | | AGACCAAGACTTTATAGTAATATACAACAGAAGAACCCCTCTAGGA | |
| | | GGCAACATCTACACAGCACCCTCTCTACACCCAGGCAATGCCATTT | |
| | | TAGCAAAACACAAAATATTAGTACCAAGTTTACAGACAAGACCAAA | |
| | | GGGTAGAAAAGCAATTAGACTAAGAATAGCACCCCCCACACTCTTT | |
| | | ACAGACAAGTGGTACTTTCAAAAGGACATAGCCGACCTCACCCTTT | |
| | | TCAACATCATGGCAGTTGAGGCTGACTTGCGGTTTCCGTTCTGCTC | |
| | | ACCACAAACTGACAACACTTGCATCAGCTTCCAGGTCCTTAGTTCC | |
| | | GTTTACAACAACTACCTCAGTATTAATACCTTTAATAATGACAACTC | |
| | | AGACTCAAAGTTAAAAGAATTTTTAAATAAAGCATTTCCAACAACAG | |
| | | GCACAAAAGGAACAAGTTTAAATGCACTAAATACATTTAGAACAGA | |
| | | AGGATGCATAAGTCACCCACAACTAAAAAAACCAAACCCACAAATA | |
| | | AACAAACCATTAGAGTCACAATACTTTGCACCTTTAGATGCCCTCT | |
| | | GGGGAGACCCCATATACTATAATGATCTAAATGAAAACAAAAGTTT | |
| | | GAACGATATCATTGAGAAAATACTAATAAAAAACATGATTACATACC | |
| | | ATGCAAAACTAAGAGAATTTCCAAATTCATACCAAGGAAACAAGGC | |
| | | CTTTTGCCACCTAACAGGCATATACAGCCCACCATACCTAAACCAA | |
| | | GGCAGAATATCTCCAGAAATATTTGGACTGTACACAGAAATAATTT | |
| | | ACAACCCTTACACAGACAAAGGAACTGGAAACAAAGTATGGATGG | |
| | | ACCCACTAACTAAAGAGAACAACATATATAAAGAAGGACAGAGCAA | |
| | | ATGCCTACTGACTGACATGCCCCTATGGACTTTACTTTTTGGATAT | |
| | | ACAGACTGGTGTAAAAAGGACACTAATAACTGGGACTTACCACTAA | |
| | | ACTACAGACTAGTACTAATATGCCCTTATACCTTTCCAAAATTGTAC | |
| | | AATGAAAAGGTAAAAGACTATGGGTACATCCCGTACTCCTACAAAT | |
| | | TCGGAGCGGGTCAGATGCCAGACGGCAGCAACTACATACCCTTTC | |
| | | AGTTTAGAGCAAAGTGGTACCCCACAGTACTACACCAGCAACAGG | |
| | | TAATGGAGGACATAAGCAGGAGCGGGCCCTTTGTACCTAAGGTAG | |
| | | AAAAACCAAGCACTCAGCTGGTAATGAAGTACTGTTTTAACTTTAA | |
| | | CTGGGGCGGTAACCCTATCATTGAACAGATTGTTAAAGACCCCAG | |
| | | CTTCCAGCCCACCTATGAAATACCCGGTACCGGTAACATCCCTAG | |
| | | AAGAATACAAGTCATCGACCCGCGGGTCCTGGGACCGCACTACTC | |
| | | GTTCCGGCCATGGGACATGCGCAGACACACATTTAGCAGAGCAAG | |
| | | TATTAAGAGAGTGTCAGAACAACAAGAAACTTCTGACCTTGTATTC | |
| | | TCAGGCCCAAAAAAGCCTCGGGTCGACATCCCAAAACAAGAAACC | |
| | | CAAGAAGAAAGCTCACATTCACTCCAAAGAGAATCGAGACCGTGG | |
| | | GAGACCGAGGAAGAAAGCGAGACAGAAGCCCTCTCGCAAGAGAG | |
| | | CCAAGAGGTCCCCTTCCAACAGCAGTTGCAGCAGCAGTACCAAGA | |
| | | ACAGCTCAAGCTCAGACAGGGAATCAAAGTCCTCTTCGAGCAGCT | |
| | | CATAAGGACCCAACAAGGGGTCCATGTAAACCCATGCCTACAGTA | |
| | | G | |
| CAF05755.1 | AJ620234.1 | ATGCACTTTTCTCGAATAAGCAGAAAGAAAAGGAAAGTGCTACTGC | 154 |
| | | TTTGCGTGCCAGCAGCTAAGAAAAAACCAACTGCTATGAGCTTCTG | |
| | | GAAACCTCCGGTACACAATGTCACGGGGATCCAACGCATGTGGTA | |
| | | TGGGTCCTTTCACCGTGGCCACGCTTCTTTTTGTGGTTGTGGGAAT | |
| | | CCTATACTTCACATTACTGCACTTGCTGAAACATATGGCCATCCAA | |
| | | CAGGCCCGAGACCTTCTGGGCCACCGGGAGTAGACCCCAACCCC | |
| | | CACATCCGTAGAGCCAGGCCTGCCCCGGCCGCTCCGGAGCCCTC | |
| | | ACAGGTTGATTCGAGACCAGCCCTGACATGGCATGGGGATGGTG | |
| | | GAAGCGACGGAGGCGCTGGTGGTCCCGGAAGCGGTGGACCCGT | |
| | | GGCAGACTTCGCAGACGATGGCCTCGATCAGCTCGTCGCCGCCC | |
| | | TAGACGACGAAGAGTAA | |
| CAF05756.1 | AJ620234.1 | ATGGCATGGGGATGGTGGAAGCGACGGAGGCGCTGGTGGTCCCG | 155 |
| | | GAAGCGGTGGACCCGTGGCAGACTTCGCAGACGATGGCCTCGAT | |
| | | CAGCTCGTCGCCGCCCTAGACGACGAAGAGTAAGGAGGCGCAGA | |
| | | CGGTGGAGGAGGGGGAGACGAAAAACAAGGACTTACAGACGCAG | |
| | | GAGACGCTTTAGACGCAGGAGACGAAAAGCAAAACTTATAATAAAA | |
| | | CTGTGA | |
| CAF05757.1 | AJ620234.1 | ATGAAAGGCCCCTTCGGGGGAGGACACAGCACTATGAGGTTCAG | 156 |
| | | CCTCTACATTTTGTTTGAGGAGCACCTCAGACACATGAACTTCTGG | |
| | | ACCAGAAGCAACGATAACCTAGAGCTAACCAGATACTTGGGGGCT | |
| | | TCAGTAAAAATATACAGGCACCCAGACCAAGACTTTATAGTAATAT | |
| | | ACAACAGAAGAACCCCTCTAGGAGGCAACATCTACACAGCACCCT | |
| | | CTCTACACCCAGGCAATGCCATTTTAGCAAAACACAAAATATTAGT | |
| | | ACCAAGTTTACAGACAAGACCAAAGGGTAGAAAAGCAATTAGACTA | |
| | | AGAATAGCACCCCCCACACTCTTTACAGACAAGTAG | |
| CAF05758.1 | | ATGGCAGTTGAGGCTGACTTGCGGTTTCCGTTCTGCTCACCACAA | 157 |
| | | ACTGACAACACTTGCATCAGCTTCCAGGTCCTTAGTTCCGTTTACA | |
| | | ACAACTACCTCAGTATTAATACCTTTAATAATGACAACTCAGACTCA | |
| | | AAGTTAAAAGAATTTTTAAATAAAGCATTTCCAACAACAGGCACAAA | |
| | | AGGAACAAGTTTAAATGCACTAAATACATTTAGAACAGAAGGATGC | |
| | | ATAAGTCACCCACAACTAAAAAAACCAAACCCACAAACAAACAAAC | |
| | | CATCAGAGTCACAATACTTTGCACCTTTAGATGCCCTCTGGGGAGA | |
| | | CCCCATATACTATAATGATCTAAATGAAAAGAAAAGTTTCAAGAATA | |
| | | TCATTGAGAACATACTAATAAAAAACATGATTACATACCATGAAAAA | |
| | | CTAACAGAATTTCCAAATTCATACCAAGGAAACAAGGCCTTTTGCC | |
| | | ACCTAACAGGCATATACAGCCCACCATACCTAAACCAAGGCAGAAT | |
| | | ATCTCCAGAAATATTTGGACTGTACACAGAAATAATTTACAACCCTT | |
| | | ACACAGACAAAGGAACTGGAAACAAAGTATGGATGGACCCACTAA | |
| | | CTAAAGAGAACAACATATATAAAGAAGGACAGAGCAAATGCCTACT | |
| | | GACTGACATGCCCCTATGGACTTTACTTTTTGGATATACAGACTGG | |
| | | TGTAAAAAGGACACTAATAACTGGGACTTACCACTAAACTACAGAC | |
| | | TAGTACTAATATGCCCTTATACCTTTCCAAAATTGTACAATGAAAAG | |
| | | GTAAAAGACTATGGGTACATCCCGTACTCCTACAAATTCGGAGCG | |
| | | GGTCAGATGCCAGACGGCAGCAACTACATACCCTTTCAGTTTAGA | |
| | | GCAAAGTGGTACCCCACAGTACTACACCAGCAACAGGTAATGGAG | |
| | | GACATAAGCAGGAGCGGGCCCTTTGCACCTAAGGTAGAAAAACCA | |
| | | AGCACTCAGCTGGTAATGAAGTACTGTTTTAACTTTAACTGGGGCG | |
| | | GTAACCCTATCATTGAACAGATTGTTAAAGACCCCAGCTTCCAGCC | |
| | | CACCTATGAAATACCCGGTACCGGTAACATCCCTAGAAGAATACAA | |
| | | GTCATCGACCCGCGGGTCCTGGGACCGCACTACTCGTTCCGGTC | |
| | | ATGGGACATGCGCAGACACACATTTAGCAGAGCAAGTATTAAGAG | |
| | | AGTGTCAGAACAACAAGAAACTTCTGACCTTGTATTCTCAGGCCCA | |
| | | AAAAAGCCTCGGGTCGACATCCCAAAACAAGAAACCCAAGAAGAA | |
| | | AGCTCACATTCACTCCAAAGAGAATCGAGACCGTGGGAGACCGAG | |
| | | GAAGAAAGCGAGACAGAAGCCCTCTCGCAAGAGAGCCAAGAGGT | |
| | | CCCCTTCCAACAGCAGTTGCAGCAGCAGTACCAAGAGCAGCTCAA | |
| | | GCTCAGACAGGGAATCAAAGTCCTCTTCGAGCAGCTCATAAGGAC | |
| | | CCAACAAGGGGTCCATGTAAACCCATGCCTACAGTAG | |
| CAF05759.1 | | ATGCACTTTTCTCGAATAAGCAGAAAGAAAAGGAAAGTGCTACTGC | 158 |
| | | TTTGCGTGCCAGCAGCTAAGAAAAAACCAACTGCTATGAGCTTCTG | |
| | | GAAACCTCCGGTACACAATGTCACGGGGATCCAACGCATGTGGTA | |
| | | TGAGTCCTTTCACCGTGGCCACGCTTCTTTTTGTGATTGTGGGAAT | |
| | | CCTATACTTCACATTACTGCACTTGCTGAAACATATGGCCATCCAA | |
| | | CAGGCCCGAGACCTTCTGGGCCACCGGGAGTAGACCCCAACCCC | |
| | | CACATCCGTAGAGCCAGGCCTGCCCCGGCCGCTCCGGAGCCCTC | |
| | | ACAGGTTGATTCGAGACCAGCCCTGACATGGCATGGGGATGGTG | |
| | | GAAGCGACAGAGGCGCTGGTGGTTCCGGAAGCGGTGGACCCGTG | |
| | | GCAGACTTCGCAGACGATGGCCTCGATCAGCTCGTTGCCGCCCTA | |
| | | GACGACGAAGAGTAA | |
| CAF05760.1 | AJ620234.1 | ATGGCATGGGGATGGTGGAAGCGACAGAGGCGCTGGTGGTTCCG | 159 |
| | | GAAGCGGTGGACCCGTGGCAGACTTCGCAGACGATGGCCTCGAT | |
| | | CAGCTCGTTGCCGCCCTAGACGACGAAGAGTAAGGAGGCGCAGA | |
| | | CGGTGGAGGAGGGGGAGACGAAAAACAAGGACTTACAGACGCAG | |
| | | GAGACGCTTTAGACGCAGGAGACGAAAAGCAAAACTTATAATAAAA | |
| | | CTGTGGCAACCTGCAGTAATTAAAAGATGCAGAATAAAGGGATACA | |
| | | TACCACTGATTATAAGTGGGAACGGTACCTTTGCCACAAACTTTAC | |
| | | CAGTCACATAAATGACAGAATAATGAAGGGCCCCTTCGGGGGAGG | |
| | | ACACAGCACTATGAGGTTCAGTCTCTACATTTTGTTTGAGGAGCAC | |
| | | CTCAGACACATGAACTTCTGGACCAGAAGCAACGATAACCTAGAG | |
| | | CTAACCAGATACTTGGGGGCTTCAGTAAAAATATACAGGCACCCA | |
| | | GACCAAGACTTTATAGTAATATACAACAGAAGAACCCCTCTAGGAG | |
| | | GCAACATCTACACAGCACCCTCTCTACACCCAGGCAATGCCATTTT | |
| | | AGCAAAACACAAAATATTAGTACCAAGTTTACAGACAAGACCAAAG | |
| | | GGTAGAAAAGCAATTAGACTAAGAATAGCACCCCCCACACTCTTTA | |
| | | CAGACAAGTGGTACTTTCAAAAGGACATAGCCGACCTCACCCTTTT | |
| | | CAACATCATGGCAGTTGAGGCTGACTTGCGGTTTCCGTTCTGCTC | |
| | | ACCACAAACTGACAACACTTGCATCAGCTTCCAGGTCCTTAGTTCC | |
| | | GTTTACAACAACTACCTCAGTATTAATACCTTTAATAATGACAACTC | |
| | | AGACTCAAAGTTAAAAGAATTTTTAAATAAAGCATTTCCAACAACAG | |
| | | GCACAAAAGGAACAAGTTTAAATGCACTAAATACATTTAGAACAGA | |
| | | AGGATGCATAAGTCACCCACAACTAAAAAAACCAAACCCACAAATA | |
| | | AACAAACCATTAGAGTCACAATACTTTGCACCTTTAGATGCCCTCT | |
| | | GGGGAGACCCCATATACTATAATGATCTAAATGAAAACAAAAGTTT | |
| | | GAACGATATCATTGAGAAAATACTAATAAAAAACATGATTACATACC | |
| | | ATGCAAAACTAAGAGAATTTCCAAATTCATACCAAGGAAACAAGGC | |
| | | CTTTTGCCACCTAACAGGCATATACAGCCCACCATACCTAAACCAA | |
| | | GGCAGAATATCTCCAGAAATATTTGGACTGTACACAGAAATAATTT | |
| | | ACAACCCTTACACAGACAAAGGAACTGGAAACAAAGTATGGATGG | |
| | | ACCCACTAACTAAAGAGAACAACATATATAAAGAAGGACAGAGCAA | |
| | | ATGCCTACTGACTGACATGCCCCTATGGACTTTACTTTTTGGATAT | |
| | | ACAGACTGGTGTAAAAAGGACACTAATAACTGGGACTTACCACTAA | |
| | | ACTACAGACTAGTACTAATATGCCCTTATACCTTTCCAAAATTGTAC | |
| | | AATGAAAAGGTAAAAGACTATGGGTACATCCCGTACTCCTACAAAT | |
| | | TCGGAGCGGGTCAGATGCCAGACGGCAGCAACTACATACCCTTTC | |
| | | AGTTTAGAGCAAAGTGGTACCCCACAGTACTACACCAGCAACAGG | |
| | | TAATGGAGGACATAAGCAGGAGCGGGCCCTTTGCACCTAAGGTAG | |
| | | AAAAACCAAGCACTCAGCTGGTAATGAAGTACTGTTTTAACTTTAA | |
| | | CTGGGGCGGTAACCCTATCATTGAACAGATTGTTAAAGACCCCAG | |
| | | CTTCCAGCCCACCTATGAAATACCCGGTACCGGTAACATCCCTAG | |
| | | AAGAATACAAGTCATCGACCCGCGGGTCCTGGGACCGCACTACTC | |
| | | GTTCCGGTCATGGGACATGCGCAGACACACATTTAGCAGAGCAAG | |
| | | TATTAAGAGAGTGTCAGAACAACAAGAAACTTCTGACCTTGTATTC | |
| | | TCAGGCCCAAAAAAGCCTCGGGTCGACATCCCAAAACAAGAAACC | |
| | | CAAGAAGAAAGCTCACATTCACTCCAAAGAGAATCGAGACCGTGG | |
| | | GAGACCGAGGAAGAAAGCGAGACAGAAGCCCTCTCGCAAGAGAG | |
| | | CCAAGAGGTCCCCTTCCAACAGCAGTTGCAGCAGCAGTACCAAGA | |
| | | GCAGCTCAAGCTCAGACAGGGAATCAAAGTCCTCTTCGAGCAGCT | |
| | | CATAAGGACCCAACAAGGGGTCCATGTAAACCCATGCCTACAGTA | |
| | | G | |
| AAC28465.1 | AF079173.1 | ATGGCCTATGGCTGGTGGCGCCGAAGGAGAAGACGGTGGCGCAG | 160 |
| | | GTGGAGACCCAGACCATGGAGGCCCCGCTGGAGGACCCGAAGAC | |
| | | GCAGACCTGCTAGACGCCGTGGCCACCGCAGAAACGTAAGAAGA | |
| | | CGCCGCAGAGGAGGGAGGTGGAGGAGGAGATATAGGAGATGGAA | |
| | | AAGAAAGGGCAGGCGCAGAAAAAAAGCTAAAATAATAATAAGACA | |
| | | ATGGCAACCAAACTACAGAAGGAGATGTAACATAGTAGGCTACATC | |
| | | CCTGTACTAATATGTGGCGAAAATACTGTCAGCAGAAACTATGCCA | |
| | | CACACTCAGACGATACCAACTACCCAGGACCCTTTGGGGGGGGTA | |
| | | TGACTACAGACAAATTTACTTTAAGAATTCTGTATGACGAGTACAAA | |
| | | AGGTTTATGAACTACTGGACAGCATCTAACGAAGACCTAGACCTTT | |
| | | GTAGATATCTAGGAGTAAACCTGTACTTTTTCAGACACCCAGATGT | |
| | | AGATTTTATCATAAAAATTAATACCATGCCTCCTTTTCTAGACACAG | |
| | | AACTCACAGCCCCTAGACTACACCCAGGCATGCTAGCCCTAGACA | |
| | | AAAGAGCAAGATGGATACCTAGCTTAAAATCTATACCAGGAAAAAA | |
| | | ACACTATATTAAAATAAGAGTAGGGGCACCAAAAATGTTCACTGAT | |
| | | AAATGGTACCCCCAAACAGATCTTTGTGACATGGTGCTTCTAACTG | |
| | | TCTATGCAACCGCAGCGGATATACCATATCCGTTCGGCTCACCACT | |
| | | AACTGACTCTGTGGTTGTGAACTTCCAGGTTCTGCAATCCATGTAT | |
| | | GATAAATACATTAGCATATTACCAGACCAAAAGTCACAAAGTAAGT | |
| | | CACTACTTAGTAACATAGCAAATTACATTCCCTTTTATAATACCACA | |
| | | CAAACTATAGCCCAATTAAAGCCATTTATAGATGCAGGCAATATAA | |
| | | CATCAGGCACAGCAGCAACAACATGGGGATCATACATAAACACAA | |
| | | CCAAATTTACTACAACAGCCACAACAACTTATACATATCCAGGCAC | |
| | | TACAACTAACACAGTTACTATGTATTCCTCTAATGACTCCTGGTACA | |
| | | GAGGAACAGTATATAACAATCAAATTAAAGAGTTACCAAAAAAAGC | |
| | | AGCTGAATTATACTCAAAAGCAACAAAAACCTTGCTAGGAAACACC | |
| | | TTCACAACTGAAGACTGCACACTAGAATACCATGGAGGACTATACA | |
| | | GCTCAATATGGCTATCCCCTGGTAGATCTTACTTTGAAACACCAGG | |
| | | AGCATACACAGACATAAAGTACAATCCATTCACAGACAGAGGAGAA | |
| | | GGCAACATGTTATGGATAGACTGGCTAAGCAAAAAAAACATGAACT | |
| | | ATGACAAAGTACAAAGTAAATGCTTAGTATCAGACCTACCTCTATG | |
| | | GGCATCAGCATATGGATATGTAGAATTTTGTGCAAAAAGTACAGGA | |
| | | GACCAGAACATACACATGAATGCCAGGCTACTAATAAGAAGTCCCT | |
| | | TTACAGACCCACAGCTACTAGTACACACAGACCCCACAAAAGGCTT | |
| | | TGTTCCCTACTCTTTAAACTTTGGAAATGGTAAAATGCCAGGAGGT | |
| | | AGTAGTAATGTGCCTATTAGAATGAGAGCTAAATGGTATCCAACAT | |
| | | TGTTTCACCAACAAGAAGTACTAGAGGCCTTAGCACAGTCAGGCC | |
| | | CCTTTGCATACCACTCAGACATTAAAGAAGTATCTCTGGGTATGAA | |
| | | ATACCGTTTTAAGTGGATCTGGGGTGGAAACCCCGTTCGCCAACA | |
| | | GGTTGTTAGAAATCCCTGCAAAGAAACCCACTCCTCGGGCAATAG | |
| | | AGTCCCTAGAAGCTTACAAATCGTTGACCCGAAATACAACTCACCG | |
| | | GAACTCACATTCCATACCTGGGACTTCAGACGTGGCCTCTTTGGC | |
| | | CCGAAAGCTATTCAGAGAATGCAACAACAACCAACAACTACTGACA | |
| | | TTTTTTCAGCAGGCCGCAAGAGACCCAGGAGGGACACCGAGGTGT | |
| | | ACCACTCCAGCCAAGAAGGGGAGCAAAAAGAAAGCTTACTTTTCC | |
| | | CCCCAGTCAAGCTCCTCAGACGAGTCCCCCCGTGGGAAGACTCG | |
| | | CAGCAGGAGGAAAGCGGGTCGCAAAGCTCAGAGGAAGAGACGCA | |
| | | GACCGTCTCCCAGCAGCTCAAGCAGCAGCTGCAGCAACAGCAAAT | |
| | | CCTGGGAGTCAAACTCAGACTCCTGTTCGACCAAGTCCAAAAAATC | |
| | | CAACAAAATCAAGATATCAACCCTACCTTGTTACCAAGGGGGGGG | |
| | | GATCTAGCATCGTTATTTCAAATAGCACCATAA | |
| AAD20024.1 | AF129887.1 | ATGGCCTATGGGTTGTGGAGGAGACGGCGAAGGAGGTGGAAGAG | 161 |
| | | GTGGAGACGCAGACGGTGGAGACGCCGCTGGAGGACCCGCCGA | |
| | | CGCAGACCTGCTGGACGCCGTAGACGCCGCAGAACAGTAAGGAG | |
| | | ACGGCGCAGGCGCGGGAGGTGGAGGAGGAGATATAGGAGATGG | |
| | | AGGCGAAAAGGCAGACGCAGGAAAAAGAAAAAACTCATAATAAGA | |
| | | CAATGGCAGCCAAACTATACCAGAAAGTGCAACATTGTGGGTTATA | |
| | | TGCCAGTTATAATGTGTGGCGAAAATACTGTCAGCAGAAACTATGC | |
| | | CACACACTCAGACGATACCAACTACCCAGGACCCTTTGGGGGGGG | |
| | | TATGACTACAGACAAATTTACTTTAAGAATTCTGTATGACTGGTACA | |
| | | AAAGGTTTATGAACTACTGGACAGCATCTAACGAAGACCTAGACCT | |
| | | TTGTAGATATCTAGGAGTGAACCTGTACTTTTTCAGACACCCAGAT | |
| | | GTAGATTTTATCATAAAAATTAATACCATGCCTCCTTTTCTAGACAC | |
| | | AGAACTCACAGCCCCTAGCATACACCCAGGCATGCTAGCCCTAGA | |
| | | CGAAAGAGCAAGATGGATACCTAGCTTAAAATCTAGACCAGGAAA | |
| | | AAAACACTATATTAAAATAAGAGTAGGGGCACCAAAAATGTTCACT | |
| | | GATAAATGGTACCCCCAAACAGATCTTTGTGACATGGTGCTTCTAA | |
| | | CTGTCTATGCAACCGCAGCGGATATGCAATATCCGTTCGGCTACC | |
| | | CACTAACTGACTCTGTGGTTGTGAACTTCCAGGTTCTGCAATCCAT | |
| | | GTATGATAAATACATTAGCATATTACCAGACCAAAAGTCACAAAGA | |
| | | GAGTCACTACTTAGTAACATAGCAAATTACATTCCCTTTTATAATAC | |
| | | CACACAAACTATAGCCCAATTAAAGCCATTTATAGATGCAGGCAAT | |
| | | ATAACATCAGGCACAACAGCAACAACATGGGGATCATACATAAACA | |
| | | CAACCAAATTTACTACAACAGCCACAACAACTTATACATATCCAGG | |
| | | CACTACAACTAACACAGTTACTATGTTAACCTCTAATGACTCCTGGT | |
| | | ACAGAGGAACAGTATATAACAATCAAATTAAAGAGTTACCAAAAAA | |
| | | AGCAGCTGAATTATACTCAAAAGCAACAAAAACCTTGCTAGGAAAC | |
| | | ACCTTCACAACTGAAGACTGCACACTAGAATACCATGGAGGACTAT | |
| | | ACAGCTCAATATGGCTATCCCCTGGTAGATCTTACTTTGAAACACC | |
| | | AGGAGCATACACAGACATGAAGTACAACCCATTCACAGACAGAGG | |
| | | AGAAGGCAACATGTTATGGATAGACTGGCTAAGCAAAAAAAACATG | |
| | | AACTATGACAAAGTACAAAGTAAATGCTTAGTATCAGACCTACCTC | |
| | | TATGGGCAGCAGCATATGGTTATTTAGAATTCTGCTCTAAAAGCAC | |
| | | AGGAGACACAAACATACACATGAATGCCAGACTACTAATAAGAAGT | |
| | | CCTTTTACAGACCCCCAGCTAATAGCACACACAGACCCCACTAAAG | |
| | | GCTTTGTACCCTATTCCTTAAACTTTGGAAATGGTAAAATGCCAGG | |
| | | AGGTAGCAGCAATGTTCCCATAAGAATGAGAGCTAAGTGGTACCC | |
| | | CACTTTATTCCACCAACAAGAAGTTCTAGAGGCCTTAGCACAGTCA | |
| | | GGACCCTTTGCTTATCACTCAGACATTAAAAAAGTATCTCTAGGCA | |
| | | TAAAATACCGTTTTAAGTGGATCTGGGGTGGAAACCCCGTTCGCC | |
| | | AACAGGTTGTTAGAAACCCCTGCAAGGAACCCCACTCCTCGGTCA | |
| | | ATAGAGTCCCTAGAAGCATACAAATCGTTGACCCGAAATACAACTC | |
| | | ACCGGAACTTACCATCCATGCCTGGGACTTCAGACGTGGCTTCTTT | |
| | | GGCCCGAAAGCTATTCAAAGAATGCAACAACAACCAACTGCTACT | |
| | | GAATTTTTTTCAGCAGGCCGCAAGAGACCCAGAAGGGACACAGAA | |
| | | GTGTATCAGTCCGACCAAGAAAAGGAGCAAAAAGAAAGCTCGCTT | |
| | | TTCCCCCCAGTCAAGCTCCTCCGAAGAGTCCCCCCATGGGAGGAC | |
| | | TCGGAACAGGAGCAAAGCGGGTCGCAAAGCTCAGAGGAAGAGAC | |
| | | CCACACCGTCTCCCAGCAGCTCAAACAGCAGCTTCAGCAGCAGCG | |
| | | GATCCTCGGCGTCAAGCTCAGAGTCCTGTTCCACCAAGTCCACAA | |
| | | AATCCAACAAAATCAACATATCAACCCTACCTTATTGCCAAGGGGT | |
| | | GGGGCCCTAGCATCCTTGTCTCAGATTGCACCATAA | |
| AAD29634.1 | AF116842.1 | ATGGCCTATGGCTTGTGGCACCGAAGGAGAAGACGGTGGCGCAG | 162 |
| | | GTGGAAACGCACACCATGGAAGCGCCGCTGGAGGACCCGAAGAC | |
| | | GCAGACCTGCTAGACGCCGTGGCCGCCGCAGAAACGTAAGGAGA | |
| | | CGCCGCAGAGGAGGGAGGTGGAGGAGGAGATATAGGAGATGGAA | |
| | | AAGAAAGGGCAGGCGCAGAAAAAAAGCTAAAATAATAATAAGACA | |
| | | ATGGCAACCAAACTACAGAAGGAGATGTAACATAGTAGGCTACATC | |
| | | CCTGTACTAATATGTGGCGAAAATACTGTCAGCAGAAATTATGCCA | |
| | | CACACTCAGACGATACCAACTACCCAGGACCCTTTGGGGGGGGTA | |
| | | TGACTACAGACAAATTTACTTTAAGAATTCTGTGTGACGAGTACAAA | |
| | | AGGTTTATGAACTACTGGACAGCATCTAACGAAGACCTAGACCTTT | |
| | | GTAGATATCTAGGAGTAAACCTGTACTTTTTCAGACACCCAGATGT | |
| | | AGATTTTATCATAAAAATTAATACCATGCCTCCTTTTCTAGACACAG | |
| | | AACTCACAGCCCCTAGCATACACCCAGGCATGCTAGCCCTAGACA | |
| | | AAAGAGCAAGATGGATACCTAGCTTAAAATCTAGACCAGGAAAAAA | |
| | | ACACTATATTAAAATAAGAGTAGGGGCACCAAAAATGTTCACTGAT | |
| | | AAATGGTACCCCCAAACAGATCTCTGTGACATGGTGCTTCTAACTG | |
| | | TCTATGCAACCACAGCGGATATGCAATATCCGTTCGGCTCACCACT | |
| | | AACTGACTCTGTGGTTGTGAACTTCCAGGTTCTGCAATCCATGTAT | |
| | | GATAAAACAATTAGCATATTACCAGACGAAAAATCACAAAGAGAAA | |
| | | TTCTACTTAACAAGATAGCAAGTTACATTCCCTTTTATAATACCACA | |
| | | CAAACTATAGCCCAATTAAAGCCATTTATAGATGCAGGCAATGTAA | |
| | | CATCAGGCGCAACAGCAACAACATGGGCATCATACATAAACACAA | |
| | | CCAAATTTACTACAGCAACCACAACAACTTATGCATATCCAGGCAC | |
| | | CAACAGACCCCCAGTAACTATGTTAACCTGTAATGACTCCTGGTAC | |
| | | AGAGGAACAGTATATAACACACAAATTCAACAGTTACCAATAAAAG | |
| | | CAGCTAAATTATACTTAGAGGCAACAAAAACCTTGCTAGGAAACAA | |
| | | CTTCACAAATGAGGACTACACACTAGAATATCATGGAGGACTGTAC | |
| | | AGCTCAATATGGCTATCCCCTGGTAGATCTTACTTTGAAACAACAG | |
| | | GAGCATACACAGACATAAAGTACAATCCATTCACAGACAGAGGAG | |
| | | AAGGCAACATGTTATGGATAGACTGGCTAAGCAAAAAAAACATGAA | |
| | | CTATGACAAAGTACAAAGTAAATGCTTAGTACGAGACCTACCTCTA | |
| | | TGGGCAGCAGCATATGGATATGTAGAATTCTGTGCAAAAAGTACAG | |
| | | GAGACAAGAACATATACATGAATGCCAGGCTACTAATAAGAAGTCC | |
| | | CTTTACAGACCCACAACTACTAGTACACACAGACCCCACAAAAGGC | |
| | | TTTGTTCCTTACTCTTTAAACTTTGGAAATGGTAAAATGCCAGGAG | |
| | | GTAGTAGTAATGTGCCTATTAGAATGAGAGCTAAATGGTATCCAAC | |
| | | ATTATTTCACCAGCAAGAAGTACTAGAGGCCTTAGCACAGTCAGGC | |
| | | CCCTTTGCATACCACTCAGACATTAAAAAAGTATCTCTGGGTATGA | |
| | | AATACCGTTTTAAGTGGATCTGGGGTGGAAACCCCGTTCGCCAAC | |
| | | AGGTTGTTAGAAATCCCTGCAAAGAAACCCACTCCTCGGGCAATA | |
| | | GAGTCCCTAGAAGCTTACAAATCGTTGACCCGAAATACAACTCACC | |
| | | GGAACTCACATTCCATACCTGGGACTTCAGACGTGGTCTCTTTGG | |
| | | CCCAAGAGCTATTCAAAGAATGCAACAACAACCAACAACTACTGAC | |
| | | ATTCTTTCAGCAGGCCGCAAGAGACCCAGAAAGGACACGGAGGTG | |
| | | TACCACCCCAGCCAAGAAGGGGAGCAAAAAGAAAGCTTACTTTTC | |
| | | CCCCCAGTCAAGCTCCTCAGACGAGTCCCCCCGTGGGAAGACTC | |
| | | GCAGCAGGAGGAAAGCGGGTCGCAAAGCTCAGAGGAAGAGACGC | |
| | | AGACCGTCTCCCAGCAGCTCAAGCAGCAGCTGCAGCAACAGCAAA | |
| | | TCCTGGGAGTCAAACTCAGACTCCTGTTCGACCAAGTCCAAAAAAT | |
| | | CCAACAAAATCAAGATATCAACCCTACCTTGTTACCAAGGGGGGG | |
| | | GGATCTAGCATCGTTATTTCAAATAGCACCATAA | |
| BAA85662.1 | AB026345.1 | ATGGCCTATGGCTGGTGGCGCCGAAGGAGAAGACGGTGGCGCAG | 163 |
| | | GTGGAGACGCAGACCATGGAGGCGCCGCTGGAGGACCCGAAGAC | |
| | | GCAGACCTGCTAGACGCCGTGGCCGCCGCAGAAACGTAAGGAGA | |
| | | CGCCGCAGAGGAGGGAGGTGGAGGAGGAGATATAGGAGATGGAA | |
| | | AAGAAAGGGCAGGCGCAGAAAAAAAGCTAAAATAATAATAAGACA | |
| | | ATGGCAACCAAACTACAGAAGGAGATGTAACATAGTAGGCTACATC | |
| | | CCTGTACTAATATGTGGCGAAAATACTGTCAGCAGAAACTATGCCA | |
| | | CACACTCAGACGATACTAACTACCCAGGACCCTTTGGGGGGGGTA | |
| | | TGACTACAGACAAATTTACTTTAAGAATTCTGTATGACGAGTACAAA | |
| | | AGGTTTATGAACTACTGGACAGCATCTAACGAAGACCTAGACCTTT | |
| | | GTAGATATCTAGGAGTAAACCTATACTTTTTCAGACACCCAGATGT | |
| | | AGATTTTATTATAAAAATTAATACCATGCCTCCTTTTCTAGACACAG | |
| | | AACTCACAGCCCCTAGCATACACCCAGGCATGCTAGCCCTAGACA | |
| | | AAAGAGCAAGATGGATACCTAGCTTAAAATCTAGACCAGGAAAAAA | |
| | | ACACTATATTAAAATAAGAGTAGGGGCACCAAAAATGTTCACTGAT | |
| | | AAATGGTACCCCCAAACAGATCTTTGTGACATGGTGCTTCTAACTG | |
| | | TCTATGCAACCGCAGCGGATATGCAATATCCGTTCGGCTCACCAC | |
| | | TAACTGACTCTGTGGTTGTGAACTTCCAGGTTCTGCAATCCATGTA | |
| | | TGATGAAAAAATTAGCATATTACCAGACCAAAAATCACAAAGAGAA | |
| | | AGCCTACTTACTAGCATAGCAAATTACATTCCCTTTTATAATACCAC | |
| | | ACAAACTATAGCCCAATTAAAGCCATTTATAGATGCAGGCAATGTA | |
| | | ACATCAGGCACAACAGCAACAACATGGGGGTCATACATAAACACA | |
| | | ACCAAGTTTACTACAACAGCCACAACAACTTATACATATCCAGGCA | |
| | | CCACCACAACCACAGTAACTATGTTAACCTCTAATGACTCCTGGTA | |
| | | CAGAGGAACAGTATATAACAACCAAATTAAAGACTTACCAAAAAAA | |
| | | GCAGCTGAATTATACTCAAAAGCAACAAAAACCTTGCTAGGAAACA | |
| | | CCTTCACAACTGAAGACTACACACTAGAATACCATGGAGGACTGTA | |
| | | CAGCTCAATATGGCTATCCCCTGGTAGATCTTACTTTGAAACACCA | |
| | | GGAGCATATACAGACATAAAGTACAATCCATTTACAGACAGAGGAG | |
| | | AAGGCAACATGTTATGGATAGACTGGCTAAGCAAAAAAAACATGAA | |
| | | CTACGACAAAGTACAGAGTAAATGCTTAATATCAGACCTACCTCTA | |
| | | TGGGCAGCAGCATATGGATATGTAGAATTTTGTGCAAAAAGTACAG | |
| | | GAGACCAGAACATACACATGAATGCCAGGCTACTAATAAGAAGTC | |
| | | CCTTTACAGACCCACAACTACTAGTACACACAGACCCCACAAAAGG | |
| | | CTTTGTTCCTTACTCTTTAAACTTTGGAAATGGTAAAATGCCAGGAG | |
| | | GTAGTAGTAATGTGCCTATTAGAATGAGAGCTAAATGGTATCCAAC | |
| | | ATTATTTCACCAGCAAGAAGTACTAGAGGCCTTAGCACAGTCAGGC | |
| | | CCCTTTGCATACCACTCAGACATTAAAAAAGTATCTCTGGGTATGA | |
| | | AATACCGTTTTAAGTGGATCTGGGGTGGAAACCCCGTTCGCCAAC | |
| | | AGGTTGTTAGAAATCCCTGCAAAGAAACCCACTCCTCGGGCAATA | |
| | | GAGTCCCTAGAAGCTTACAAATCGTTGACCCGAAATACAACTCACC | |
| | | GGAACTCACATTCCATACCTGGGACTTCAGACGTGGCCTCTTTGG | |
| | | CCCGAAAGCTATTCAGAGAATGCAACAACAACCAACAACTACTGAC | |
| | | ATTTTTTCAGCAGGCCGCAAGAGACCCAGGAGGGACACCGAGGT | |
| | | GTACCACTCCAGCCAAGAAGGGGAGCAAAAAGAAAGCTTACTTTT | |
| | | CCCCCCAGTCAAGCTCCTCAGACGAGTCCCCCCGTGGGAAGACT | |
| | | CGCAGCAGGAGGAAAGCGGGTCGCAAAGCTCAGAGGAAGAGACG | |
| | | CAGACCGTCTCCCAGCAGCCCAAGCAGCAGCTGCAGCAACAGCG | |
| | | AATCCTGGGAGTCAAACTCAGACTCCTGTTCAACCAAGTCCAAAAA | |
| | | ATCCAACAAAATCAAGATATCAACCCTACCTTGTTACCAAGGGGGG | |
| | | GGGATCTAGCATCCTTATTTCAAGTAGCACCATAA | |
| BAA85664.1 | AB026346.1 | ATGGCCTATGGCTGGTGGCGCCGAAGGAGAAGACGGTGGCGCAG | 164 |
| | | GTGGAGACGCAGACCATGGAGGCGCCGCTGGAGGACCCGAAGAC | |
| | | GCAGACCTGCTAGACGCCGTGGCCGCCGCAGAAACGTAAGGAGA | |
| | | CGCCGCAGAGGAGGGAGGTGGAGGAGGAGATATAGGAGATGGAA | |
| | | AAGAAAGGGCAGGCGCAGAAAAAAAGCTAAAATAATAATAAGACA | |
| | | ATGGCAACCAAACTACAGAAGGAGATGTAACATAGTAGGCTACATC | |
| | | CCTGTACTAATATGTGGCGAAAATACTGTCAGCAGAAACTATGCCA | |
| | | CACACTCAGACGATACCAACTACCCAGGACCCTTTGGGGGGGGTA | |
| | | TGACTACAGACAAATTTACTTTAAGAATTCTGTATGACGAGTACAAA | |
| | | AGGTTTATGAACTACTGGACAGCATCTAACGAAGATCTAGACCTTT | |
| | | GTAGATATCTAGGAGTAAACCTGTACTTTTTCAGACACCCAGATGT | |
| | | AGATTTTATCATAAAAATTAATACCATGCCTCCTTTTCTAGACACAG | |
| | | AACTCACAGCCCCTAGCATACACCCAGACATGCTAGCCCTAGACA | |
| | | AAAGAGCAAGATGGATACCTAGCTTAAAATCTAGACCGGGAAAAAA | |
| | | ACACTATATTAAAATAAGAGTTGGGGCACCAAAAATGTTCACTGAT | |
| | | AAATGGTACCCCCAAACAGATCTTTGTGACATGGTGCTTCTAACTG | |
| | | TCTATGCAACCACAGCGGATATGCAATATCCGTTCGGCTCACCACT | |
| | | AACTGACTCTGTGGTTGTGAACTTCCAGGTTCTGCAATCCATGTAT | |
| | | GATGAAAACATTAGCATATTACCAACCGAAAAATCAAAAAGAGATG | |
| | | TCCTACATAGTACTATAGCAAATTACACTCCCTTTTATAATACCACA | |
| | | CAAATTATAGCCCAATTAAGGCCATTTGTAGATGCAGGCAATCTAA | |
| | | CATCAGCGTCAACAACAACAACATGGGGATCATACATAAACACAAC | |
| | | CAAGTTTAATACAACAGCCACAACAACTTATACATATCCAGGCAGC | |
| | | ACGACAACCACAGTAACTATGTTAACCTGTAATGACTCCTGGTACA | |
| | | GAGGAACAGTATATAACAATCAAATTAGCAAGTTACCAAAACAAGC | |
| | | AGCTGAATTTTACTCAAAAGCAACAAAAACCTTGCTAGGAAACACG | |
| | | TTCACAACTGAGGACCACACACTAGAATACCATGGAGGACTGTAC | |
| | | AGCTCAATATGGCTATCCGCTGGTAGATCTTACTTTGAAACACCAG | |
| | | GAGCATATACAGACATAAAGTATAATCCATTCACAGACAGAGGAGA | |
| | | AGGCAACATGTTATGGATAGACTGGCTAAGCAAAAATAACATGAAC | |
| | | TATGACAAAGTACAAAGTAAATGCTTAATATCAGACCTACCTCTATG | |
| | | GGCAGCAGCATATGGATATGTAGAATTTTGTGCAAAAAGTACAGGA | |
| | | GACCAGAACATACACATGAATGCCAGACTACTAATAAGAAGTCCCT | |
| | | TTACAGACCCACAACTACTAGTACACACAGACCCCACAAAAGGCTT | |
| | | TGTTCCTTACTCTTTAAACTTTGGAAATGGTAAAATGCCAGGAGGT | |
| | | AGTAGTAATGTGCCTATTAGAATGAGAGCTAAATGGTATCCAACAT | |
| | | TATTTCACCAGCAAGAAGTACTAGAGGCCTTAGCACAGTCAGGCC | |
| | | CCTTTGCATACCACTCAGACATTAAAAAAGTATCTCTGGGTATGAA | |
| | | ATACCGTTTTAAGTGGATCTGGGGTGGAAACCCCGTTCGCCAACA | |
| | | GGTTGTTAGAAATCCCTGCAAAGAAACCCACTCCTCGGGCAATAG | |
| | | AGTCCCTAGAAGCTTACAAATCGTTGACCCGAAATACAACTCACCG | |
| | | GAACTCACATTCCATACCTGGGACTTCAGACGTGGCCTCTTTGGC | |
| | | CCGAAAGCTATTCAGAGAATGCAACAACAACCAACAACTACTGACA | |
| | | TTTTTTCAGCAGGCCGCAAGAGACCCAGGAGGGACACCGAGGTGT | |
| | | ACCACTCCAGCCAAGAAGGGGAGCAAAAAGAAAGCTTACTTTTCC | |
| | | CCCCAGTCAAGCTCCTCAGACGAGTCCCCCCGTGGGAAGACTCG | |
| | | CAGCAGGAGGAAAGCGGGTCGCAAAGCTCAGAGGAAGAGACGCA | |
| | | GACCGTCTCCCAGCAGCTCAAGCAGCAGCTGCAGCAACAGCGAAT | |
| | | CCTGGGAGTCAAACTCAGACTCCTGTTCAACCAAGTCCAAAAAATC | |
| | | CACCAAAATCAAGATATCAACCCTACCTTGTTACCAAGGGGGGGG | |
| | | GATCTAGCATCCTTATTTCAAATAGCACCATAA | |
| BAA85666.1 | AB026347.1 | ATGGCCTATGGCTGGTGGCGCCGAAGGAGAAGACGGTGGCGCAG | 165 |
| | | GTGGAGACGCAGACCATGGAGGCGCCGCTGGAGGACCCGAAGAC | |
| | | GCAGACCTGCTAGACGCCGTGGCCGCCGCAGAAACGTAAGGAGA | |
| | | CGCCGCAGAGGAGGGAGGTGGAGGAGGAGATATAGGAGATGGAA | |
| | | AAGAAAGGGCAGGCGCAGAAAAAAAGCTAAAATAATAATAAGACA | |
| | | ATGGCAACCAAACTACAGAAGGAGATGTAACATAGTAGGCTACATC | |
| | | CCTGTACTAATATGTGGCGAAAATACTGTCAGCAGAAACTATGCCA | |
| | | CACACTCAGACGATACCAACTACCCAGGACCCTTTGGGGGGGGTA | |
| | | TGACTACAGACAAATTTACTTTAAGAATTCTGTATGACGAGTACAAA | |
| | | AGGTTTATGAACTACTGGACAGCATCTAACGAAGATCTAGACCTTT | |
| | | GTAGATATCTAGGAGTAAACCTGTACTTTTTCAGACACCCAGATGT | |
| | | AGATTTTATCATAAAAATTAATACCATGCCTCCTTTTCTAGACACAG | |
| | | AACTCACAGCCCCTAGCATACACCCAGGCATGCTAGCCCTAGACA | |
| | | AAAGAGCAAGATGGATACCTAGCTTAAAATCTAGACCGGGAAAAAA | |
| | | ACACTATATTAAAATAAGAGTTGAGGCACCAAAAATGTTCACTGATA | |
| | | AATGGTACCCCCAAACAGATCTTTGTGACATGGTGCTTCTAACTGT | |
| | | CTATGCAACCACAGCGGATATGCAATATCCGTTCGGCTCACCACTA | |
| | | ACTGACTCTGTGGTTGTGAACTTCCAGGTTCTGCAATCCATGTATG | |
| | | ATCAAAACATTAGCATATTACCAACCGAAAAATCAAAGAGAACACA | |
| | | ACTACATGATAATATAACAAGGTACACTCCCTTTTATAATACCACAC | |
| | | AAACTATAGCCCAATTAAAGCCATTTGTAGATGCAGGCAATGTAAC | |
| | | ACCAGTGTCACCAACAACAACATGGGGATCATACATAAACACAACC | |
| | | AAGTTTACTACAACAGCCACAACAACTTATACATATCCAGGCACCA | |
| | | CGACAACCACAGTAACTATGTTAACCTGTAATGACTCCTGGTACAG | |
| | | AGGAACAGTATATAACAATCAAATTAGCCAGTTACCAAAAAAAGCA | |
| | | GCTGAATTTTACTCAAAAGCAACAAAAACCTTGCTAGGAGACACGT | |
| | | TCACAACTGAGGACTACACACTAGAATACCATGGAGGACTGTACA | |
| | | GCTCAATATGGCTATCCGCTGGTAGATCTTACTTTGAAACACCAGG | |
| | | AGTATATACAGACATAAAGTATAATCCATTCACAGACAGAGGAGAA | |
| | | GGCAACATGTTATGGATAGACTGGCTAAGCAAAAAAAACATGAACT | |
| | | ATGACAAAGTACAAAGTAAATGCTTAATATCAGACCTACCTCTATG | |
| | | GGCAGCAGCATATGGATATGTAGAATTTTGTGCAAAAAGTACAGGA | |
| | | GACCAAAACATACACATGAATGCCAAACTACTAATAAGAAGTCCCT | |
| | | TTACAGACCCACAACTACTAGTACACACAGACCCCACAAAAGGCTT | |
| | | TGTTCCTTACTCTTTAAACTTTGGAAATGGTAAAATGCCAGGAGGT | |
| | | AGTAGTAATGTGCCTATTAGAATGAGAGCTAAATGGTATCCAACAT | |
| | | TATTTCACCAGCAAGAAGTACTAGAGGCCTTAGCACAGTCAGGCC | |
| | | CCTTTGCATACCACTCAGACATTAAAAAAGTATCTCTGGGTATGAA | |
| | | ATACCGTTTTAAGTGGATCTGGGGTGGAAACCCCGTTCGCCAACA | |
| | | GGTTGTTAGAAATCCCTGCAAAGAAACCCACTCCTCGGGCAATAG | |
| | | AGTCCCTAGAAGCTTACAAATCGTTGACCCGAAATACAACTCACCG | |
| | | GAACTCACATTCCATACCTGGGACTTCAGACGTGGCCTGTTTGGC | |
| | | CCGAAAGCTATTCAGAGAATGCAACAACAACCAACAACTACTGACA | |
| | | TTTTTTCAGCAGGCCGCAAGAGACCCAGGAGGGACACCGAGGTGT | |
| | | ACCACTCCAGCCAAGAAGGGGAGCAAAAAGAAAGCTTACTTTTCC | |
| | | TCCCAGTCAAGCTCCTCAGACGAGTCCCCCCGTGGGAAGACTCGC | |
| | | AGCAGGAGGAAAGCGGGTCGCAAAGCTCAGAGGAAGAGACGCAG | |
| | | ACCGTCTCCCAGCAGCTCAAGCAGCAGCTGCAGCAACAGCGAATC | |
| | | CTGGGAGTCAAACTCAGACTCCTGTTCAACCAAGTCCAAAAAATCC | |
| | | AACAAAATCAAGATATCAACCCTACCTTGTTACCAAGGGGGGGGG | |
| | | ATCTGGCATCCTTATTTCAAATAGCACCATAA | |
| BAA90406.1 | AB030487.1 | ATGGCCTATGGGTGGTGGAGGAGACGCCGCAGAAGGTGGAAGAG | 166 |
| | | ATGGAGGAGAAGGCCCAGGTGGAGACGCCCATGGAGGACCCGCA | |
| | | GACGCAGACCTGCTAGACGCCGTGGACGCCGCAGAACAGTAAGG | |
| | | AGACGGGAGCGCGGGAGGTGGAGGAGGCGCTATAGGAGGTGGA | |
| | | GGAAAAAGGGCAAACGCAGGATAAAAAAGAAACTTATAATAAGACA | |
| | | GTGGCAGCCAAACTATACCAGAAAGTGCGACATATTAGGCTACAT | |
| | | GCCTGTAATCATGTGTGGAGAGAACACTCTAATAAGAAACTATGCC | |
| | | ACACACGCAAACGACTGCTACTGGCCGGGACCCTTTGGGGGCGG | |
| | | CATGGCCACCCAGAAATTCACACTCAGAATCCTGTACGATGACTAC | |
| | | AAGAGGTTTATGAACTACTGGACCTCCTCAAACGAGGACCTAGAC | |
| | | CTCTGTAGATACAGGGGAGTCACCCTGTACTTTTTCAGACACCCAG | |
| | | ATGTAGACTTTATCATCCTGATAAACACCACACCTCCGTTCGTAGA | |
| | | TACAGAGATCACAGGACCCAGCATACATCCTGGCATGATGGCCCT | |
| | | CAACAAGAGAGCCAGGTTCATCCCCAGCCTAAAAACTAGACCTGG | |
| | | CAGAAGACACATAGTAAAGATTAGAGTGGGGGCCCCCAAACTGTA | |
| | | CGAGGACAAATGGTACCCCCAGTCAGAACTCTGTGACATGCCCCT | |
| | | GCTAACCGTCTACGCGACCGCAGCGGATATGCAATATCCGTTCGG | |
| | | CTCACCACTAACTGACACTCCTGTTGTAACCTTCCAAGTGTTGCGC | |
| | | AGCATGTACAACGACGCCCTTAGCATACTTCCCTCTAACTTTGAAC | |
| | | AGGACGACAATGCAGGCCAAAAACTTTACAATGAAATATCATCATA | |
| | | TTTACCATACTACAACACCACAGAAACAATAGCACAACTAAAGAGA | |
| | | TATGTAGAAAATACAGAAAAAATTTCCACAACACCAAACCCATGGC | |
| | | AATCAAATTATGTAAACACTATTACCTTCACCACTGCACAAAGTATT | |
| | | ACAACTACAACCCCATACACCACCTTCTCAGACAGCTGGTACAGG | |
| | | GGCACAGTATACAAAAACGCAATCACTAAAGTGCCACTTGCCGCA | |
| | | GCTAAACTTTATGAAACCCAAACAAAAAACCTGCTGTCTCCAACAT | |
| | | TTACAGGAGGGTCCGAGTACCTAGAATACCATGGAGGCCTGTACA | |
| | | GCTCCATATGGCTATCAGCAGGCCGATCCTACTTTGAAACAAAGG | |
| | | GAGCATACACAGACATATGCTACAACCCCTACACAGACAGGGGAG | |
| | | AAGGGAACATGTTGTGGATAGACTGGCTATCCAAAGGAGATTCCA | |
| | | GATATGACAAAGCACGCAGCAAATGTCTAATAGAAAAACTACCTAT | |
| | | GTGGGCCGCAGTATATGGGTACGCAGAATACTGTGCAAAAGCCAC | |
| | | AGGAGACTCTAACATAGACATGAACGCCAGAGTAGTAATGAGGTG | |
| | | TCCATACACCGTACCCCAAATGATAGACACAAGCGATCCCCTCAG | |
| | | AGGCTTTATACCCTATAGCTTTAACTTTGGAAAGGGAAAAATGCCT | |
| | | GGAGGAACAAATCAAGTCCCCATAAGAATGAGAGCTAAGTGGTAC | |
| | | CCTTGTCTCTTTCACCAAAAAGAAGTTCTAGAAGCTATAGGACAGT | |
| | | CAGGCCCCTTCGCCTACCATAGTGATCAGAAAAAAGCAGTACTAG | |
| | | GCCTAAAATACAGATTTCACTGGATATGGGGTGGAAACCCCGTGTT | |
| | | TCCACAGGTTGTTAGAAACCCCTGCAAAGACACCCAAGGTTCCAC | |
| | | AGGCCCTAGAAAGCCTCGCTCAGTACAAATCATTGACCCGAAGTA | |
| | | CAACACACCAGAGCTTACCATCCACGCGTGGGATTTCAGACGTGG | |
| | | CTTCTTTGGCCCAAAAGCTATTAAAAGAATGCAACAACAACCAACA | |
| | | GATGCTGAACTTCTTCCACCAGGCCGCAAGAGGAGCAGGAGAGA | |
| | | CACCGAAGTCCTGCAAAGCAGCCAAGAAAGGCAAAAAGAAAGCTT | |
| | | ACTTTTACAACAGCTCCACCTCCAGGGACGAGTACCCCCGTGGGA | |
| | | AAGCTTGCAAGGGTTGCAGACAGAAACAGAAAGCCAAAAAGAGCA | |
| | | CGAGGGCACCCTTTCCCAGCAGATCAGAGAGCAGGTTCAGCAGC | |
| | | AGAAGCTCCTCGGGAGACAGCTCAGAGAAATGTTCTTACAACTCC | |
| | | ACAAAATCCTACAAAATCAACACGTCAACCCTACCTTATTGCCAAG | |
| | | GGATCAGGGTTTAATTTGGTGGTTTCAGATTCAGTAA | |
| BAA90409.1 | AB030488.1 | ATGGCTTATGGGTGGTGGAGGAGACGCCGCAGGAGGTGGAAGAG | 167 |
| | | ATGGAGGAGAAGGCCCAGGTGGAGACGCCCATGGAGGACCCGCA | |
| | | GACGCAGACCTGCTGGACGCCGTGGACGCCGCAGAACAGTAAGG | |
| | | AGACGGAGGCGCGGGAGGTGGAGGAGGCGCTATAGGAGGTGGA | |
| | | GGAAAAAGGGCAGACGCAGGAGAAAAAAGAAACTTATAATAAGAC | |
| | | AATGGCAGCCAAACTATACCAGAAAGTGCAACATAGTTGGTTACAT | |
| | | GCCAGTCATCATGTGTGGAGAGAACACTCTAATCAGAAACTATGCC | |
| | | ACACACGCATACAACTGCTCCTGGCCGGGACCCTTTGGGGGCGG | |
| | | CATGGCCACCCAAAAATTTACTCTGAGAATACTGTACGATGACTAC | |
| | | AAAAGATTTATGAACTACTGGACCTCCTCAAACGAGGACCTAGACC | |
| | | TGTGCAGATATAGAGGAGCTACACTGTACTTTTTCAGAGACCCAGA | |
| | | TGTAGACTTTATTATACTGATAAACACCACTCCTCCATTTGTAGACA | |
| | | CAGAGATTACAGGGCCCAGCATACATCCCGGCATGCTGGCACTCA | |
| | | ACAAGAGAGCAAGATTTATACCCAGCTTAAAGACTAGACCCAGCA | |
| | | GAAGACACATAGTAAAGATCAGAGTGGGGGCCCCCAAACTGTATG | |
| | | AGGACAAGTGGTACCCCCAGTCAGAACTTTGTGACATGCCCCTGC | |
| | | TAACCGTCTATGCGACCGCAACGGATATGCAATATCCGTTCGGCT | |
| | | CACCACTAACTGACACTCCTATTGTAACCTTCCAAGTGTTGCGCAG | |
| | | CATGTACAACGACGCCCTTAGCATACTTCCCTCTAACTTTGAAGGT | |
| | | GACGACAGTGCAGGCGCAAAACTTTACAAACAAATATCAGAATACA | |
| | | TACCATACTATAACACCACAGAAACAATAGCACAGTTAAAGGGATA | |
| | | TGTAGAAAACACAGAAAAAACCCAAACAACACCTAATCCATGGCAA | |
| | | TCAAAATATGTAAACACAAAACCATTTGACACTGCACAAACAATTAC | |
| | | AAACCAAAAGCCATACACTCCATTCGCAGACACATGGTACAGGGG | |
| | | CACAGCATACAAAGAAGAAATTAAAAATGTACCACTAAAAGCAGCC | |
| | | GAACTGTATGAATTACATACTACACACCTGTTATCTACAACATTCAC | |
| | | AGGAGGGTCCAAATACTTAGAATACCATGGAGGCTTATACAGCTC | |
| | | CATATGGCTGTCAGCAGGCCGCTCCTACTTTGAAACAAAAGGAGC | |
| | | ATACACAGACATTTGCTACAACCCCTACACAGACAGGGGAGAAGG | |
| | | CAACATGGTGTGGATAGACTGGCTAGTAAAGACAGACTCTAGATAT | |
| | | GACAAGACACGCAGCAAATGCCTTATAGAAAAACTACCTCTATGGG | |
| | | CTGCAGTATACGGGTACGCAGAGTACTGCGCCAAGGCCACAGGA | |
| | | GACTCTAACATAGACATGAACGCCAGAGTAGTTATCAGGAGCCCC | |
| | | TACACTACACCTCAAATGATAGACACCAACGACTCTCTAAGAGGCT | |
| | | TTATAGTATACAGCTTTAACTTTGGAAAGGGAAAAATGCCTGGAGG | |
| | | AACAAATCAAGTCCCCATAAGAATGAGAGCTAAGTGGTACCCTTGC | |
| | | CTCTTTCACCAAAAAGAAGTTCTAGAAGCTATAGGACAGTCAGGCC | |
| | | CCTTCGCCTACCATAGTGATCAGAAAAAAGCAGTACTAGGCCTAAA | |
| | | ATACAGATTTCACTGGATATGGGGTGGAAACCCCGTGTTTCCACA | |
| | | GGTTGTTAGAAACCCCTGCAAAGACACCCAAGGTTCCACAGGCCC | |
| | | TAGAAAGCCTCGCTCAGTACAAATCATTGACCCGAAGTACAACACA | |
| | | CCAGAGCTTACCATCCACGCGTGGGATTTCAGACGTGGCTTCTTT | |
| | | GGCCCAAAAGCTATTAAAAGAATGCAACAACAACCAACAGATGCT | |
| | | GAACTTCTTCCACCAGGCCGCAAGAAGAGCAGGAGAGACACCGAA | |
| | | GTCCTGCAAAGCAGCCAAGAAAGGCAAAAAGAAAGCTTACTTTTCC | |
| | | AACAGCTCCAGCTCCAGCGACGAGTACCCCCGTGGGAAAGCTCG | |
| | | CAAGGGTCGCAGACAGAAACAGAAAGCCAAAAAGAGCAGGAGGG | |
| | | CACCCTCTCCCAGCAGCTCAGAGAGCAGCTTCAGCAGCAGAAGCT | |
| | | CCTCGGCAGACAGCTCAGGGAAATGTTCCTACAAATCCACAAAAT | |
| | | CCTACAAAATCAACAAGTCAACCCTATTTTATTGCCAAGGGATCAG | |
| | | GCTTTAATTTCCTGGTTTCAGATTCAGTAA | |
| BAA90412.1 | AB030489.1 | ATGGCCTATGGGTGGTGGAGGAGACGCCGCAGGAGGTGGAAGAG | 168 |
| | | ATGGAGGAGAAGGCCCAGGTGGAGACGCCGCTGGAGGACCCGC | |
| | | AGACGCAGACCTGCTGGACGCCGTAGACGCCGCAGAACAGTAAG | |
| | | GAGACGCAGGCGCGGGAGGTGGAGGAGCAGATATAGGAGATGGA | |
| | | GGCGAAAGGGCAGACGCAGGCGAAAAGAAAAACTAATAATAAGAC | |
| | | AATGGCAGCCAAACTATACCAGAAAGTGCAACATTGTGGGTTACAT | |
| | | GCCAGTAATCATGTGTGGAGAAAATACTGTTATCAGAAACTATGCC | |
| | | ACACACACATACGACTGCTCCTGGCCAGGACCCTTTGGGGGCGG | |
| | | CATGGCCACCCAAAAATTTACTCTGAGAATACTGTACGATGACTAC | |
| | | AAAAGATTTATGAACTACTGGACCTCCTCAAACGAGGACCTAGATC | |
| | | TCTGCAGATACAGAGGAGCAACCCTATACTTTTTCAGAGACCCAGA | |
| | | TGTAGACTTTATTATACTTATAAACACTACTCCTCCATTTGTAGACA | |
| | | CAGAAATAACAGGGCCCAGCATACACCCAGGCATGCTGGCACTAA | |
| | | ACAAAAGAGCTAGATTCATTCCCAGTCTAAAAACCAGACCAGGCAG | |
| | | GAGACACATAGTAAAAATAAAAGTAGGGGCCCCTAGAATGTATGAA | |
| | | GACAAGTGGTACCCCCAGTCAGAACTTTGTGACATGCCCCTCCTA | |
| | | ACGATCTATGCAACCGCAACGGATATGCAACATCCGTTCGGCTCA | |
| | | CCACTAACTGACACTCCTGTTGTAACCTTCCAAGTGTTGCGCAGCA | |
| | | TGTACAACGACGCCCTTAGCATACTTCCCTCTAACTTTGAAGACGA | |
| | | TTCAAGTCCAGGGGCTGCACTTTACAAACAAATATCAGAATACATA | |
| | | CCATACTATAACACCACAGAAACAATAGCACAGCTAAAGAGATATG | |
| | | TAGAAAACACAGAAAAAACCCAAACAACACTTAATCCATGGCAATC | |
| | | AAGATATGTAAACACAACACTATTTAACACTGCAGAAACAATTGCA | |
| | | AACCAAAAGCCATACACTAAATTCGCAGACACATGGTACAGGGGC | |
| | | ACAGCATACAAAGACGCAATTAAAGACATACCACTAAAAGCAGCC | |
| | | GAATTGTATGTAAACCAAACCAAATACCTGTTATCTACAACATTCAC | |
| | | AGGAGGGTCCAAATACTTAGAATACCATGGAGGCTTATACAGCTC | |
| | | CATATGGCTGTCAGCAGGCCGCTCCTACTTTGAAACAAAAGGAGC | |
| | | ATACACAGACATTTGCTACAACCCCTACACAGACAGGGGAGAAGG | |
| | | CAACATGGTGTGGATAGACTGGCTATCGAAAACAGACTCAAAATAT | |
| | | GACAAGACCCGCAGCAAATGCCTTATAGAAAAACTGCCGCTATGG | |
| | | GCATCGGTATACGGGTACGCAGAATACTGTGCCAAGGCCACAGGA | |
| | | GACTCTAACATAGACATGAACGCCAGAGTAGTTATAAGATGCCCCT | |
| | | ACACTACACCTCAAATGATAGACACCACCGACCCAACTAGAGGGT | |
| | | TCATAGTATACAGCTTTAACTTTGGTAAGGGCAAAATGCCGGGAGG | |
| | | TAGCAATGAAGTACCCATAAGAATGAGAGCCAAATGGTACCCCTG | |
| | | CCTCTTTCACCAAAAAGAGGTCCTAGAAGCCATAGGCCAGTCAGG | |
| | | CCCCTTTGCTTATCACAGCGATCAAAAAAAAGCAGTTTTAGGTTTA | |
| | | AAATACAAATTTCACTGGATATGGGGTGGAAACCCCGTGTTCCCAC | |
| | | AGGTTATTAAAAACCCCTGCAAAAACACTCAATTTTCCACAGGCCC | |
| | | TAGAAAGCCTCGCTCATTACAAATCATTGACCCGAATTACAACACA | |
| | | CCAAAGCTTACCATCCACGCTTGGGATTTCAGACTTGGCTTCTTTG | |
| | | GCCCAAAAGCTATTAAAAGAATGCAACAACAACCAACAGATGCTGA | |
| | | ACTTCTTCCACCAGGCCGCAAGAGGAGCAGGAGAGACACCGAAG | |
| | | TCCTGCAAAGCAGCCAAGAAAGGCAAAAAGGAAACTTACTTTTCCA | |
| | | ACAGTTCCAGCTCCAGCGACGAGTACCCCCGTGGGAAAGCTCGC | |
| | | AAGGGTCGCAGACAGGAACACAAAGCCAAAAAGAGCAGGAGGGC | |
| | | ACCCTCTCCCAGCAGCTCAGAGAGCAGCTTCAGCAGCAGAAGCTC | |
| | | CTCGGCAGACAGCTCAGGGAAATGTTCCTACAACTCCACAAAATC | |
| | | CAACAAAATCAACACGTCAACCCTACCTTATTGCCAAGGGATCAGG | |
| | | CTTTAATTTGCTGGTTTCAGATTCAGTAA | |
| BAA90825.1 | AB038340.1 | ATGGCCTATGGCTGGTGGCGCCGAAGGAGAAGACGGTGGCGCAG | 169 |
| | | GTGGAGACGCAGACCATGGAGGCGCCGCTGGAGGACCCGAAGAC | |
| | | GCAGACCTGCTAGACGCCGTGGCCGCCGCAGAAACGTAAGGAGA | |
| | | CGCCGCAGAGGAGGGAGGTGGAGGAGGAGATATAGGAGATGGAA | |
| | | AAGAAAGGGCAGGCGCAGAAAAAAAGCTAAAATAATAATAAGACA | |
| | | ATGGCAACCAAACTACAGAAGGAGATGTAACATAGTAGGCTACATC | |
| | | CCTGTACTAATATGTGGCGAAAATACTGTCAGCAGAAACTATGCCA | |
| | | CACACTCAGACGATACTAACTACCCAGGACCCTTTGGGGGGGGTA | |
| | | TGACTACAGACAAATTTACTTTAAGAATTCTGTATGACGAGTACAAA | |
| | | AGGTTTATGAACTACTGGACAGCATCTAACGAAGACCTAGACCTTT | |
| | | GTAGATATCTAGGAGTAAACCTATACTTTTTCAGACACCCAGATGT | |
| | | AGATTTTATTATAAAAATTAATACCATGCCTCCTTTTCTAGACACAG | |
| | | AACTCACAGCCCCTAGCATACACCCAGGCATGCTAGCCCTAGACA | |
| | | AAAGAGCAAGATGGATACCTAGCTTAAAATCTAGACCAGGAAAAAA | |
| | | ACACTATATTAAAATAAGAGTAGGGGCACCAAAAATGTTCACTGAT | |
| | | AAATGGTACCCCCAAACAGATCTTTGTGACATGGTGCTTCTAACTG | |
| | | TCTATGCAACCGCAGCGGATATGCAATATCCGTTCGGCTCACCAC | |
| | | TAACTGACTCTGTGGTTGTGAACTTCCAGGTTCTGCAATCCATGTA | |
| | | TGATGAAAAAATTAGCATATTACCAGACCAAAAATCACAAAGAGAA | |
| | | AGCCTACTTACTAGCATAGCAAATTACATTCCCTTTTATAATACCAC | |
| | | ACAAACTATAGCCCAATTAAAGCCATTTATAGATGCAGGCAATGTA | |
| | | ACATCAGGCACAACAGCAACAACATGGGGGTCATACATAAACACA | |
| | | ACCAAGTTTACTACAACAGCCACAACAACTTATACATATCCAGGCA | |
| | | CCACCACAACCACAGTAACTATGTTAACCTCTAATGACTCCTGGTA | |
| | | CAGAGGAACAGTATATAACAACCAAATTAAAGACTTACCAAAAAAA | |
| | | GCAGCTGAATTATACTCAAAAGCAACAAAAACCTTGCTAGGAAACA | |
| | | CCTTCACAACTGAAGACTACACACTAGAATACCATGGAGGACTGTA | |
| | | CAGCTCAATATGGCTATCCCCTGGTAGATCTTACTTTGAAACACCA | |
| | | GGAGCATATACAGACATAAAGTACAATCCATTTACAGACAGAGGAG | |
| | | AAGGCAACATGTTATGGATAGACTGGCTAAGCAAAAAAAACATGAA | |
| | | CTACGACAAAGTACAGAGTAAATGCTTAATATCAGACCTACCTCTA | |
| | | TGGGCAGCAGCATATGGATATGTAGAATTTTGTGCAAAAAGTACAG | |
| | | GAGACCAGAACATACACATGAATGCCAGGCTACTAATAAGAAGTC | |
| | | CCTTTACAGACCCACAACTACTAGTACACACAGACCCCACAAAAGG | |
| | | CTTTGTTCCTTACTCTTTAAACTTTGGAAATGGTAAAATGCCAGGAG | |
| | | GTAGTAGTAATGTGCCTATTAGAATGAGAGCTAAATGGTATCCAAC | |
| | | ATTATTTCACCAGCAAGAAGTACTAGAGGCCTTAGCACAGTCAGGC | |
| | | CCCTTTGCATACCACTCAGACATTAAAAAAGTATCTCTGGGTATGA | |
| | | AATACCGTTTTAAGTGGATCTGGGGTGGAAACCCCGTTCGCCAAC | |
| | | AGGTTGTTAGAAATCCCTGCAAAGAAACCCACTCCTCGGGCAATA | |
| | | GAGTCCCTAGAAGCTTACAAATCGTTGACCCGAAATACAACTCACC | |
| | | GGAACTCACATTCCATACCTGGGACTTCAGACGTGGCCTCTTTGG | |
| | | CCCGAAAGCTATTCAGAGAATGCAACAACAACCAACAACTACTGAC | |
| | | ATTTTTTCAGCAGGCCGCAAGAGACCCAGGAGGGACACCGAGGT | |
| | | GTACCACTCCAGCCAAGAAGGGGAGCAAAAAGAAAGCTTACTTTT | |
| | | CCCCCCAGTCAAGCTCCTCAGACGAGTCCCCCCGTGGGAAGACT | |
| | | CGCAGCAGGAGGAAAGCGGGTCGCAAAGCTCAGAGGAAGAGACG | |
| | | CAGACCGTCTCCCAGCAGCCCAAGCAGCAGCTGCAGCAACAGCG | |
| | | AATCCTGGGAGTCAAACTCAGACTCCTGTTCAACCAAGTCCAAAAA | |
| | | ATCCAACAAAATCAAGATATCAACCCTACCTTGTTACCAAGGGGGG | |
| | | GGGATCTAGCATCCTTATTTCAAGTAGCACCATAA | |
| BAA93586.1 | AB038622.1 | ACGGCTTGGTGGTGGGGCAGATGGAGGCGCCGCTGGAGGCCTC | 170 |
| | | GCTATCGCAGACGCACCTGGAGGGTACGAAGAAGACGACCTAGA | |
| | | CGAACTTTTCGCCGCCGCCGCCGAGGACGATATGTGAGTAGGCG | |
| | | GAGGCGCCGCCGCTACTACAGGCGCAGACTGAGACGGGGCAGAC | |
| | | GCAGAGGGCGACGAAAGAGACACAGACAGACTCTAGTCCTCAGA | |
| | | CAGTGGCAACCAGACATTGTCAGACACTGTAAAATTACAGGATGG | |
| | | ATGCCCCTTATCATCTGTGGCTCAGGGAGCACACAGAACAATTTTA | |
| | | TAACTCACATGGACGACTTTCCTCCCATGGGCTACTCCTTCGGGG | |
| | | GCAACTTTACAAACCTCTCCTTCTCCTTAGAGGGCATTTATGAACA | |
| | | ATTTCTGTACCACAGAAACAGGTGGTCTCGCTCCAACCATGACCTA | |
| | | GACCTAGCCAGATACAAAGGCACAACTCTAAAACTCTACAGACACC | |
| | | ACACCTTAGACTACATAGTCAGCTACAACAGAACAGGCCCTTTCCA | |
| | | GATCAGTGACATGACCTACCTCAGCACACACCCTGCACTCATGCT | |
| | | ACTCCAGAAACACAGAATAGTAGTACCCAGCCTACTCACTAAACCT | |
| | | AAAGGCAAGAGATCCATAAAAGTTAGAATAAAGCCACCAAAACTCA | |
| | | TGCTCAACAAATGGTACTTCACCAAAGACATATGCAGCATGGGCCT | |
| | | CTTCCAACTACAGGCCACAGCATGCACCCTATACAACCCCTGGCT | |
| | | CAGAGACACCACAAAAAGCCCAGTCATAGGCTTCAGAGTACTTAAA | |
| | | AACAGTATTTATACAAACCTCAGCAACCTACCAGAACATGATCAAA | |
| | | CCAGACAAGCCATTAGACGAAAACTACACCCAGACTCCTTAACAG | |
| | | GATCAACTCCATATCAAAAAGGCTGGGAATACAGCTACACAAAACT | |
| | | AATGGCTCCAATATACTATCAAGCAAATAGAAACAGCACATACAAC | |
| | | TGGCTAAATTATCAAACAAACTATGCTCAAACATTCACCAAATTTAA | |
| | | AGAAAAAATGAATGAAAACCTTGCACTAATTCAAAAAGAGTATTCAT | |
| | | ACCACTATCCCAACAATGTCACTACAGACCTTATTGGCAAAAACAC | |
| | | CCTCACACATGACTGGGGTATATACAGTCCCTACTGGCTAACACC | |
| | | CACCAGAATAAGCCTAGACTGGGAAACACCCTGGACATATGTCAG | |
| | | ATACAATCCACTAGCAGACAAGGGCATAGGCAATGCTGTCTATGC | |
| | | ACAATGGTGCTCAGAACAGACCAGTAAATTAGATACAAAAAAGAGC | |
| | | AAGTGCATAATGAAAGACCTGCCACTGTGGTGCATATTTTATGGCT | |
| | | ATGTAGATTGGATAATAAAATCCACAGGAGTCAGCAGCGCAGTCA | |
| | | CTGACATGAGAGTAGCCATCATCAGCCCCTACACCGAACCAGCAC | |
| | | TTATAGGGTCAAGTCCAGACGTAGGCTACATTCCAGTAAGTGACAC | |
| | | CTTTTGCAATGGAGACATGCCGTTTCTTGCTCCATACATCCCTGTG | |
| | | GGCTGGTGGATCAAATGGTACCCTATGATTGCACACCAAAAGGAA | |
| | | GTGTTTGAGGCAATAGTTAACTGTGGACCGTTTGTGCCCAGAGAC | |
| | | CAGACCACTCCCAGTTGGGAAATTACCATGGGTTACAAAATGGACT | |
| | | GGTTATGGGGTGGCTCTCCCCTGCCTTCACAGGCAATCGACGACC | |
| | | CCTGCCAGAAGCCCACCCACGAACTACCCGATCCCGATAGACACC | |
| | | CTCGCATGTTACAAGTCTCTGACCCGACAAAGCTCGGACCGAAGA | |
| | | CAGTGTTCCACAAATGGGACTGGAGACGTGGGATGCTTAGCAAAA | |
| | | GAAGTATTAAAAGAGTCCAGGAGGACTCAACAGATGATGAATATGT | |
| | | TGCAGGGCCTTTACCAAGAAAAAGAAACAAATTCGATACCAGAGC | |
| | | CCAAGGGCTGCAAACCCCCGAAAAAGAAAGCTACACTTTACTCCA | |
| | | AGCCCTCCAAGAGTCGGGGCAAGAGACCAGCTCAGAAGACCAAG | |
| | | AACAAGCACCCCAAGAAAAAGAGGGTCAGAAGGAAGCGCTCATG | |
| | | GAGCAGCTCCAGCTCCAGAAACAGCACCAGCGAGTCCTCAAGCG | |
| | | AGGCCTCAAACTCCTCCTCGGAGACGTCCTCCGACTCCGGAGAG | |
| | | GAGTCCACTGGGACCCCCTCCTGTCATAA | |
| BAA93589.1 | AB038623.1 | ACGGCGTGGTGGTGGGGCAGATGGAGGCGTCGATGGAGGCCTC | 171 |
| | | GCTATCGCAAACGCACCTGGAGATTACGGAGACGACGACCTAGAC | |
| | | GAACTTTTCGCCGCCGCCGCCGAAGACAATATGTGAGTAGGCGGA | |
| | | GGCGCCGCCGCTACTACAGGCGCAGACTGAGACGGGGCAGACG | |
| | | CAGAGGGCGACGAAAGAGACACAGACAGACTCTAGTCCTCAGACA | |
| | | ATGGCAACCAGACGTTGTTAGACACTGTAAAATTACAGGATGGATG | |
| | | CCCCTTATCATCTGTGGCTCCGGGAGCACACAGAACAATTTTATAA | |
| | | CTCACATGGACGACTTTCCTCCCATGGGCTACTCCTTTGGGGGCA | |
| | | ACTTTACAAACCTCACCTTCTCCTTAGAGGGCATATATGAACAATTT | |
| | | CTGTACCACAGAAACAGGTGGTCTCGCTCCAACCATGACCTAGAC | |
| | | CTAGCCAGATACAAAGGCACAACTCTAAAACTCTACAGACACCACA | |
| | | CCTTAGACTACATAGTCAGCTACAACAGAACAGGCCCCTTCCAGAT | |
| | | CAGTGACATGACCTACCCCAGCACACACCCTGCACTTATGCTACT | |
| | | CCAGAAACACAGAATAGTAGTGCCCAGCGTACTCACTAAACCTAAA | |
| | | GGCAAGAGATCCATAAAGGTCAGAATAAAGCCACCAAAACTCATG | |
| | | CTTAACAAGTGGTACTTCACCAAAGACATATGCAGCATGGGCCTTT | |
| | | TTCAACTACAGGCCACAGCATGCACCCTATACAATCCCTGGCTCA | |
| | | GAGACACCACAAAAAGCCCAGTCATAGGCTTCAGGGTACTTAAAA | |
| | | ACAGTATCTATACAAACCTCAGCAACCTACCAGACCATGAGGGTTC | |
| | | CAGAGAAGCCATAAGAAAAAAACTACACCCACAATCCTTAACAGGA | |
| | | CACTCTCCCAACCAAAAAGGCTGGGAATACAGCTATACTAAACTAA | |
| | | TGGCTCCAATATACTACTCTGCCAACAGAAACAGTACATATAACTG | |
| | | GCTAAACTATCAAGACAACTATGTAGCCACATATACTAAATTCAAAG | |
| | | TCAAAATGACAGACAACTTACAACTAATACAAAAAGAATACTCATAC | |
| | | CACTATCCCAACAATACCACTACAGACCTTATTAAGAACAACACCC | |
| | | TTACACATGACTGGGGCATATACAGTCCCTACTGGCTAACACCCAC | |
| | | CAGAATAAGCCTAGACTGGGAAACACCCTGGACATATGTAAGATA | |
| | | CAACCCACTGGCAGACAAAGGCATAGGCAATGCTGTCTACGCACA | |
| | | GTGGTGCTCAGAACAGACAAGCAAATTAGACCCAAAAAAGAGCAA | |
| | | GTGCATAATGAGAGACCTGCCACTGTGGTGCATATTTTATGGCTAT | |
| | | GTAGATTGGATAGTAAAATCCACAGGAGTCAGCAGCGCAGTCACT | |
| | | GACATGAGAGTAGCCATTAGAAGCCCCTACACTGAACCAGCACTT | |
| | | ATAGGGTCAACTGAAGATGTAGGCTTCATTCCAGTAAGTGACACCT | |
| | | TTTGCAACGGAGACATGCCGTTTCTTGCTCCATACATTCCTGTGGG | |
| | | CTGGTGGATCAAGTGGTACCCCATGATTGCACACCAAAAGGAAGT | |
| | | GTTTGAGCAAATAGTAAACTGTGGACCGTTTGTGCCCAGAGACCA | |
| | | GACCACTCCCAGTTGGGAAATTACCATGGGTTACAAAATGGACTG | |
| | | GTTATGGGGTGGCTCTCCCCTGCCTTCACAGGCAATCGACGACCC | |
| | | CTGCCAGAAGCCCACCCACGAACTACCCGATCCCGATAGACACCC | |
| | | TCGCATGTTACAAGTCTCTGACCCGACAAAGCTCGGACCGAAGAC | |
| | | AGTGTTCCACAGATGGGACTGGAGACGTGGGATGCTTAGCAAAAG | |
| | | AAGTATTAAAAGAGTCCAGGAGGACTCAACAGATGATGAATATGTT | |
| | | GCAGGGCCTTTACCAAGAAAAAGAAACAAGTTCGATACCAGAGCC | |
| | | CAAGGGCTCCAAAGCCCCGAAAAAGAAAGCTACACTTTACTCCAA | |
| | | GCCCTCCAAGAGTCGGGGCAAGAGAGCAGCTCAGAAGACCAAGA | |
| | | ACAAGCACCCCAAGAAAAAGAGGGTCAGAAGGAAGCGCTCATGG | |
| | | AGCAGCTCCAGCTCCAGAAACAGCACCAGCGAGTCCTCAAGCGA | |
| | | GGCCTCAAACTCCTCCTCGGAGACGTTCTCCGACTCCGGAGAGGA | |
| | | GTACACTGGGACCCCCTCCTGTCATAA | |
| BAA93592.1 | AB038624.1 | ACGGCGTGGTGGTGGGGCAGATGGAGGCGCCGCTGGAGGCCTC | 172 |
| | | GCTATCGCAGACGCACCTGGAGGGTACGCAGAAGACGACCTAGA | |
| | | CGAACTTTTCGCCGCCGCCGCCGAGGACGATATGTGAGTAGGCG | |
| | | GAGGCGCCGCCGCTACTACAGGCGCAGACTCAGACGGGGCAGAC | |
| | | GCAGAGGGCGACGAAAGAGACACAGACAGACTCTAGTCCTCAGA | |
| | | CAATGGCAACCAGACGTTCTTAGACGCTGTAAAATTACAGGATGGA | |
| | | TGCCCCTTATCATCTGTGGCTCCGGAAGCACACAGAACAATTTTAT | |
| | | AACTCACATGGACGACTTTCCTCCCATGGGCTACTCCTACGGGGG | |
| | | CAACTTTACAAACCTCACCTTCTCCTTAGAGGGCATATATGAACAA | |
| | | TTTCTGTACCACAGAAACAGGTGGTCTCGCTCCAACCATGACCTAG | |
| | | ACCTAGCCAGATACAAAGGCACAACTCTAAAACTCTACAGACACCA | |
| | | CACCTTAGACTACATAGTGAGCTACAATAGAACAGGCCCTTTCCAG | |
| | | ATCAGTGACATGACCTACCTCAGCACACACCCTGCACTTATGCTAC | |
| | | TCCAGAAACACAGAATAGTAGTGCCCAGCCTACTCACTAAACCTAA | |
| | | AGGCAAGAGATCCATAAAAGTTAGAATAAAACCACCAAAACTCATG | |
| | | CTTAACAAGTGGTACTTCACCAAAGACATATGCAGCATGGGCCTTT | |
| | | TTCAACTACAGGCCACAGCATGCACCCTATACAACCCCTGGCTCA | |
| | | GAGACACCACAAAAAGCCCAGTCATAGGCTTCAGGGTACTTAAAA | |
| | | ACAGTATTTATACAAACCTCAGCAACCTACCAGACCATGAAGGAGC | |
| | | CAGAGAGGCCATAAGAAAAAAACTACACCCACAATCCTTAACAGG | |
| | | ATCTGTCCCAAACCAAAAAGGTTGGGAATACAGCTACACAAAACTA | |
| | | ATGGCTCCCATTTACTACCAAGCCATTAGAAACAGCACATACAACT | |
| | | GGCTAAACTATCAACAAAATTACTCACAAACATACCAAACCTTTAAA | |
| | | CAAAAAATGCAAGACAACTTACAACTAATACAAAAAGAATACATGTA | |
| | | CCACTACCCAAACAATGTAACAACAGACATACTAGGCAAAAACACA | |
| | | CTTACACATGACTGGGGCATATACAGTCCCTACTGGCTAACACCCA | |
| | | CCAGAATCAGCCTAGACTGGGAAACACCTTGGACATATGTTAGATA | |
| | | CAATCCACTAGCAGACAAGGGCATAGGCAATGCTGTCTATGCACA | |
| | | GTGGTGCTCAGAACAGACCAGTAACTTAGATACAAAAAAGAGCAA | |
| | | GTGCATAATGAAAGACCTGCCACTGTGGTGCATATTTTATGGCTAT | |
| | | GTAGATTGGGTAGTAAAATCCACAGGCGTCAGCAGCGCAGTGACT | |
| | | GACATGAGAGTAGCCATCATTAGCCCCTACACTGAACCAGCACTTA | |
| | | TAGGGTCAAGTCCAGAGGTAGGCTACATTCCAGTAAGTGACACCT | |
| | | TTTGCAATGGAGACACGCCGTTTCTTGCTCCATACATCCCTGTGGG | |
| | | CTGGTGGATCAAGTGGTACCCCATGATTGCACACCAAAAGGAAGT | |
| | | GTTTGAGGCAATAGTAAACTGTGGACCGTTTGTGCCCAGAGACCA | |
| | | GACCACTCCCAGTTGGGAAATTACCATGGGTTACAAAATGGACTG | |
| | | GTTATGGGGTGGCTCTCCCCTGCCTTCACAGGCAATCGACGACCC | |
| | | CTGCCAGAAGCCCACCCACGAACTACCCGATCCCGATAGACACCC | |
| | | TCGCATGTTACAAGTCTCTGACCCGACAAAGCTCGGACCGAAGAC | |
| | | AGTGTTCCACAAATGGGACTGGAGACGTGGGATGCTTAGCAAAAG | |
| | | AAGTATTAAAAGAGTCCAGGAGGACTCAACAGATGATGAATATGTT | |
| | | GCAGGGCCTTTACCAAGAAAAAGAAACAAGTTCGATACCAGAGCC | |
| | | CAAGGGCTCCAAAGCCCCGAAAAAGAAAGCTACACTTTACTCCAA | |
| | | GCCCTCCAAGAGTCGGGGCAAGAGACGAGCTCAGAAGACCAAGA | |
| | | ACAAGCACCCCAAGAAAAAGAGGGTCAGAAGGAAGCGCTCATGG | |
| | | AGCAGCTCCAGCTCCAGAAACAGCACCAGCGAGTCCTCAAGCGA | |
| | | GGCCTCAAACTCCTCCTCGGAGACGTTCTCCGACTCCGGAGAGGA | |
| | | GTACACTGGGACCCCCTCCTGTCATAA | |
| AAF71533.1 | AF254410.1 | ATGGCACAGGGGAGGCGCAGATACAGACGGGGTTGGCAACGCAG | 173 |
| | | GGTGTATCTGAGACGCAGGAGACGCAGGAGACGAAAGAGACTTG | |
| | | TACTGACTCAGTGGCACCCCGCAGTTAGGAGAAAATGCACCATCA | |
| | | CGGGGTACATGCCCGTGGTGTGGTGCGGACACGGCAGGGCCAG | |
| | | CTACAACTACGCCTGGCATTCAGATGACTGTATAAAACAGCCCTGG | |
| | | CCCTTTGGAGGGTCTCTGTCCACCGTGTCCTTTAACCTTAAAGTAC | |
| | | TGTATGACGAAAACCAGAGGGGACTTAACAGATGGACGTACCCCA | |
| | | ACGATCAGCTAGACCTCGGCCGCTACAAGGGCTGCAAACTAACAT | |
| | | TCTACAGAACCAAAAATACCAACTACCCAGGACCCTTTGGGGGGG | |
| | | GTATGACTACAGACAAATTTACTTTAAGAATTCTGTATGACGAGTAC | |
| | | AAAAGGTTTATGAACTACTGGACAGCATCTAACGAAGACCTAGACC | |
| | | TTTGTAGATATTTAGGAGTAAACCTGTACATTTTCAGACACCCAGAT | |
| | | GTAGATTTTATCATAAAAATTAATACCATGCCTCCTTTTCTAGACAC | |
| | | AGAAATCACAGCCGCTAGCATACACCCAGGCATACTAGCCCTAGA | |
| | | CAAAAGAGCAAGATGGATACCTAGCTTAAAATCTAGACCAGGAAAA | |
| | | AAACACTATATTAAAATAAGAGTAGGGGCACCAAAAATGTTCACTG | |
| | | ATAAATGGTACCCCCAAACAGATCTCTGTGACATGGTGCTTCTAAC | |
| | | TATCTATGCAACCGCAGCGGATATGCAATATCCGTTCGGCTCACCA | |
| | | CTAACTGACACTGTGGTTGTGAACTTCCAGGTTCTGCAATCCATGT | |
| | | ATGATGAAAACATTAGCATATTACCAGACCAAAAGACACAAAGAGA | |
| | | GAAACTACTTACTAGCATATCAAACTACATTCCCTTTTATAATACCA | |
| | | CACAAACTATAGCCCAATTGAAGCCATTTGTAGATGCAGGCAATAA | |
| | | AGTATCAGGCACAACAACAACAACATGGGCATCATACATAAACACA | |
| | | ACCAGATTTACTACAACAGCCACAACAACTTATACATATCCAGGCT | |
| | | CTACCACTAACACAGTAACTATGTTAACCTCTAATGACTCCTGGTA | |
| | | CAGAGGAACAGTATATAACAATCAAATTAAAAACTTACCAAAACAA | |
| | | GCAGCTGAATTATACTCAAAAGCAACAAAAACCTTGCTAGGAAACA | |
| | | CCTTCACAACTGAAGACTACACACTAGAATACCATGGAGGACTGTA | |
| | | CAGCTCAATATGGCTATCCCCTGGTAGATCTTACTTTGAAACACCA | |
| | | GGAGCATACACAGATATAAAGTACAATCCATTTACAGACAGAGGAG | |
| | | AAGGCAACATGTTATGGATAGACTGGCTAAGCAAAAAAAACATGAA | |
| | | CTATGACAAAGTACAAAGTAAATGCTTAGTATCAGACCTACCTCTAT | |
| | | GGGCAGCAGCATATGGATATGTAGAATTTTGTGCAAAAAGTACAG | |
| | | GAGACCAGAACATACACATGAATGCCAGGCTACTAATAAGAAGTC | |
| | | CCTTTACAGACCCACAGCTACTAGTACACACAGACCCCACAAAAG | |
| | | CCTTTGTTCCCTACTCTTTAAACTTTGGAAATGGTAAAATGCCAGG | |
| | | AGGTAGTAGTAATGTGCCTATTAGAATGAGAGCTAAATGGTATCCC | |
| | | ACTTTATTCCACCAACAAGAAGTTCTAGAGGCTTTAGCGCAGTCAG | |
| | | GACCCTTCGCTTATCACTCAGACATTAAAAAAGTATCTCTAGGCAT | |
| | | AAAATACCGTTTTAAGTGGATCTGGGGTGGAAACCCCGTTCGCCA | |
| | | ACAGGTTGTTAGAAATCCCTGCAAGGAACCCCACTCCTCGGGCAA | |
| | | TAGAGTCCCTAGAAGCATACAAATCGTTGACCAGAAATACAACTCA | |
| | | CCGGAACTTACCATCCATTCCTGGGACTTCAGACGTGGCTTCTTTG | |
| | | GCCCGAAAGCTATTCAAAGAATGCAACAACAACCAACTGCTACTGA | |
| | | ATTTTTTTCAGCAGGCCGCAAGAGACCCAGAAGGGACACAGAAGT | |
| | | ATATCAGTCCGACCAAGAAAAGGAGCAAAAAGAAAGCTCGCTTTTC | |
| | | CCCCCAGTCAAGCTCCTCCGAAGAGTCCCCCCGTGGGAGGACTC | |
| | | GGACAGGAAGCAAAGCGGGTCGCAAAGCTCAGAGGAAGAGACGC | |
| | | AGACCGTCTCCCAGCAGCTCAAGCAGCAGCTGCAGCAACAGCGA | |
| | | ATCCTGGGAGTCAAACTCAGACTCCTGTTCTACCAAATCCAAAGAA | |
| | | TCCAACAAAATCAAGATATCAACCCTACCTTGTTACCAAGGGGGGG | |
| | | GGATCTAGCATCCTTATTTCAAATAGCATAA | |
| BAB19928.1 | AB050448.1 | ATGGCGTGGACCTGGTGGTGGCAGAGGAGGCGCCGAAGGTGGC | 174 |
| | | CGTGGAGAAGGAGAAGGTGGAGAAGACTACGCACAAGAAGACCT | |
| | | AGACGCCTTGTTCGACGCCGTCGCAAGAGATACAGAGTAAGGAGA | |
| | | CGGAGGCGGTGGGGAAGGAGACGTGGGCGACGCACATACCTTAG | |
| | | ACGCGGACTTAAAAAGAGAAAAAGGAGAAAAAAACTCAGACTGAC | |
| | | TCAGTGGAACCCTAGCACAATTAGGGGATGTACAATTAAGGGAAT | |
| | | GGCGCCCCTAATAGTGTGCGGCCACACCATGGCTGGCAATAACTT | |
| | | TGCCATCCGAATGGAGGACTATGTATCTCAGATTAAACCGTTCGGA | |
| | | GGGTCCTTCAGTACCACCACCTGGAGCTTAAAAGTACTGTGGGAC | |
| | | GAGCACACCAGATTCCACAACACCTGGAGCTACCCAAACACTCAG | |
| | | CTAGACTTAGCCAGGTTCAAAGGAGTAACCTTCTACTTCTACAGAG | |
| | | ACAAAGACACAGACTTTATTATAACCTATAGCTCCGTGCCACCTTTT | |
| | | AAAATAGACAAATACTCCTCAGCCATGCTACACCCAGGCATGCTTA | |
| | | TGCAGAGAAAAAAGAAGATATTATTACCCAGCTTTACAACCAGACC | |
| | | TAGGGGCAGAAAAAAAGTTAAAGTACACATAAAACCTCCTGTCTTA | |
| | | TTTGAAGACAAATGGTACACCCAGCAGGACCTGTGCGACGTTAAT | |
| | | CTTTTGTCACTTGCGGTTTCTGCGGCTTCCTTTAGACATCCGTTCT | |
| | | GCCCACCACAAACTGACAACATTTGCATAACCTTCCAGGTGTTGAA | |
| | | AGACAAGTATTACACACAAATGTCAGTTACACCAGATACCGCAGGT | |
| | | ACAAAAAAAGACGACGAAATTCTTGACCACTTATACTCAACTGCAG | |
| | | AATACTATCAAACTGTTCACACACAAGGAATAATTAACAAAACACAA | |
| | | AGAGTAGCTAAATTCTCCACCTCTAATAATACCCTAGGTGACCAAA | |
| | | GTGAGATATCATTATATTTAAACCAACCAACAACAACTAACATAGGA | |
| | | AACACGTTATCCACAGGCCATAACTCAGTGTATGGCTTTCCATCAT | |
| | | ACAACCCACAAAAAGACAAACTTAGAAAAATAGCAGACTGGTTTTG | |
| | | GACACAGGAAGCCAACAAAGAGAATGTAGTTACAGGCTCATACTC | |
| | | AATGCCTACTAACAAAGCAGTAGGCTATCACCTAGGAAAATATAGC | |
| | | CCTATATTCCTAAGTTCATACAGAACCAACCTACAATTTAGAACAGC | |
| | | ATACACAGACGTTACATACAACCCACTAAATGACAAAGGTAAAGGC | |
| | | AATGAAATTTGGGTACAATATGTAACAAAACCAGACACTGTGTTCA | |
| | | ACCCCACACAGTGTAAATGCCATGTAATAGATTTACCCTTGTGGTC | |
| | | AGCATTCCATGGATACATAGACTTTGTACAAAGTGAACTAGGAATT | |
| | | CAAGAAGAAATACTAAACATTGCCATTATAGTAGTTATATGTCCATA | |
| | | CACAAAACCTAAACTAGTACATGAGACAAACCCAAAACAAGGCTTT | |
| | | GTATTCTATGACACTCAATTTGGAGACGGTAAAATGCCAGAGGGCT | |
| | | CAGGCCTAGTACCGATATACTACCAAAACAGATGGTATCCTAGAAT | |
| | | AAAGTTTCAGAGTCAAGTAGTGCATGACTTTATACTAACAGGCCCC | |
| | | TTTAGCTACAAAGATGACCTAAAAAGCACAGTACTAACAGTAGAAT | |
| | | ACAAGTTCAAATTCTTATGGGGCGGCAATATGATTCCCGAACAGGT | |
| | | TATCAGAAACCCTTGTAAAACAGAAGGACACGATCTCCCTCACACC | |
| | | AGTAGACTCCATCGCGACTTACAAGTTGTTGACCCACACACCGTG | |
| | | GGCCCCCAATGGGCGCTCCACACCTGGGACTGGCGACGTGGACT | |
| | | CTTTGGTTCAGAGGCTATCAAAAGAGTGTCTGAACAACAAGTACAT | |
| | | GATGAACTGTATTACCCACCTTCAAAGAAACCTCGATTCCTCCCTC | |
| | | CAATATCAGGCCTCCAAGAGCAAGAAAGAGACTACAGTTCGCAGG | |
| | | AGGAGAAAGAACAGTCCTCCTCAGAAGAAGAGACGGACCCGAAG | |
| | | AAAAAAGAGCAAAAACAGCAGCAGCGACTCCACCTCCAGTTCCAA | |
| | | GAGCAGCAGCGACTCGGAAACCAACTCCGACTCATCTTCCGAGAG | |
| | | CTACAGAAAACCCAAGCGGGTCTCCACTTAAATCCTATGTTATCAA | |
| | | ACCGGCTGTAA | |
| AAK01940.1 | AY026465.1 | ATGGCATGGGGATGGTGGAAGCGACGGCGGCGCTGGTGGTTCCG | 175 |
| | | GAAGCGGTGGACCCGTGGCAGACTTCGCAGACGATGGCCTAGAC | |
| | | CAGCTCGTCGGCGCCCTAGACGACGAAGAGTAAGGAGACGCAGA | |
| | | CGATGGAGGAGGGGGCGACCTAGACGCAGACTGTACCGACGCTA | |
| | | CAGACGCAAAAAACGTAGGAGACGAAAGCCCAAAATAATCTTAAAA | |
| | | CAATGGCAGCCAGACATTGTAAAGAGGTGCTACATAGTGGGCTAC | |
| | | ATTCCTGCCATAATATGCGGGGCGGGCACCTGGTCCCACAACTAC | |
| | | ACCAGCCACCTTCTAGACATTATCCCCAAAGGACCCTTTGGAGGG | |
| | | GGACACAGCACCATGAGATTCTCTCTAAAAGTGCTCTTCGAAGAG | |
| | | CACCTCAGACACCTAAACTTTTGGACACGTAGTAACCAGGATCTAG | |
| | | AACTTGTAAGATACTTCAGATGCTCCTTTAGGTTTTACAGAGACCA | |
| | | ACACACAGACTACTTAGTGCACTACAACAGAAAAACACCCCTGGG | |
| | | AGGCAACAGACTGACAGCACCTAGCCTTCACCCAGGGGTGCAGAT | |
| | | GCTAAGCAAAAACAAAATAATAGTACCCAGCTATGATACTAAACCT | |
| | | TGTAGACTTTATTATCCTTATAAACACCATGTCGCCCTTCCTCGACA | |
| | | CCCAGCTCACAGGCCCCAGCATACACCCGGGACTAATGGCCCTTA | |
| | | ACAAGAGAGCCAGATGGATCCCCAGCCTAAAAAGCAGACCGGGTA | |
| | | GAAAGCACGTAGTTAAAATTAGAGTAGGCGCTCCCAGAATGTTCAC | |
| | | AGATAAATGGTACCCCCAGTCAGATCTGTGTGACCTCCCCCTACTA | |
| | | ACTATCTTTGCCAGTGCAGCGGATATGCAATATCCGTTCGGCTCAC | |
| | | CACTAACTGACTCTGTGGTTGTGGGTTTCCAGGTTCTGCAATCCAT | |
| | | GTACAATGACTGCCTTAGCATACTTCCTGAAAATTTTAACGGCAAT | |
| | | GGCAAAGGCAAAGCTTTACATGACAACATAACTAAGTATCTCCCTA | |
| | | ACTATAACACTACTCAAACACTAGCTCAGCTAAAACCGTACATAGA | |
| | | TAACACATCCACAGGAAGCACAAATAACTGGAGCAGCTATGTAAAT | |
| | | ACATCAAAATTTACAACTGCTTCAAAAACCATTACAACCTCAGCAGA | |
| | | AGGCCCATACTATACTTTCGCAGATACCTGGTACAGAGGCACTGC | |
| | | ATACAACAATAGCATTACGAACGTTCCTTTACAGGCAGCACAACTA | |
| | | TATCACGACACAACCAAAAAACTACTAGGCACAACATTTACAGGAG | |
| | | GGTCCCCCTACCTAGAATACCACGGAGGCCTTTACTCCTCCATTTG | |
| | | GCTATCTGCAGGTCGCTCCTACTTTGAAACAAAAGGCACATACACA | |
| | | GATATAACCTACAACCCTTTTACAGACAGAGGACAAGGTAACATGG | |
| | | TATGGATAGACTGGGTATCCAAATATGACTCAGTTTACTCTAAAAC | |
| | | ACAAAGCAAATGCCTTATAGAAAACCTGCCACTGTGGGCATCAGTA | |
| | | TATGGATACGCAGAATACTGCAGCAAATCCACAGGAGACACAAAC | |
| | | ATAGAACAAAACTGCAGAGTAGTTATAAGAAGCCCCTTCACTAACC | |
| | | CTCAGCTGCTAGACCATAACAACCCACTAAGAGGGTACGTTCCCT | |
| | | ACTCCATAAACTTTGGCAACGGAAAAATGCCTGGGGGAAGCAGTC | |
| | | AGGTCCCCATAAGAATGAGAAGCAAGTGGTACCCTACTCTATTTCA | |
| | | CCAAAAAGAAGTGTTAGAGGCCATAGCGCAGGCGGGCCCCTTCG | |
| | | CGTACCACAGTGATCAGATGAAAGTGTCACTAGGCATGAAATACG | |
| | | CCTTTAAGTGGGTGTGGGGTGGCAACCCCGTATCCCAACAGGTTG | |
| | | TTAGAAACCCCTGCAAGGACACCGGTGTTTCCTCGGGCAATAGAG | |
| | | TCCCTCGATCAGTACAAATCGTTGACCCGAAGTACAACACTCCAGA | |
| | | ACTTGCAATACATGCCTGGGACTTCAGACGTGCCTGTTTGGCCCA | |
| | | AAAGCTATTAAGAGAATGCAAACAGAACCGTACCCTACTGAACTTC | |
| | | TTTCGCCAGGGCGAAAAAGATACAGGAGAGACACAGAAGCTCTAC | |
| | | TCCCCAGCCAAGAAGAACAACAAAAAGAAAACTTATTTTTCCTCCC | |
| | | AATCAAGCAGCTCCGACCAATCCCCCGTTGGAGGAGTCGGACCAA | |
| | | AGCCAAAGCGAGGAAGAGGGGGTCCAACAAGAGACGCAGACACT | |
| | | CTCCCAGCAGCTCCAGCAGCAGCTCAAGGAGCAGCAGCTCATGG | |
| | | GGGTCCAACTCCGAGCCCTGTACCAACAATTACAACGGGTCCAAC | |
| | | AAAACACACATATCGACCCTACCTTTTTGCAAGGGGGGCGGGCGT | |
| | | AACATCTTTATTTCAAACAGCGTAG | |
| AAK11696.1 | AF345521.1 | ATGGCGTGGTGGGGCAGATGGAGAAGGTGGCCGCGGCGCCGGT | 177 |
| | | GGAGGAGATGGCGGCGCCGCCGTAGAAGGAGACTACCAACAAGA | |
| | | AGAACTCGACGAGCTGTTCGCGGCCTTGGAAGACGACCAAGAAAG | |
| | | ACGGTAAGGAGACGCCGGCGCCGACCCAGACGCACTTACCGACG | |
| | | GGGGTGGCGACGCAGACGGTACATAAGACGCAGGAGGGGACGC | |
| | | AGAAAGAAACTGACTCTGACTATGTGGAACCCCAACATAGTGAGG | |
| | | AGATGTAACATAGAGGGAGGGCTGCCTCTAATACTGTGTGGAGAA | |
| | | AACAGGGCCGCATTTAACTACGCCTACCACTCAGAGGACTACACA | |
| | | GAGCAGCCATTCCCCTTCGGTGGAGGAATGAGCACCACCACATTC | |
| | | TCACTGAGAGGCCTCTATGACCAGTACACAAAACACATGAACAGAT | |
| | | GGACGTTCTCAAACGACCAGCTAGACCTCGCCAGATACAGGGGCT | |
| | | GCAAATTCAGGTTTTACAGACACCCCACCTGTGACTTTATAGTGCA | |
| | | CTACAACCTGGTTCCTCCTCTAAAGATGAACCAGTTCACCAGTCCC | |
| | | AACACGCACCCGGGACTCCTCATGCTGACTAAACACAAAATAATAA | |
| | | TACCCAGCTTCTTAACAAGACCAGGGGGTCGCAGATTCGTAAAGA | |
| | | TCAGACTGCCCCCCCCTAAGCTGTTTGAAGACAAGTGGTACACCC | |
| | | AGCAGGACTTGTGCAAACAACCGTTAGTTACTCTAACCGCAACCG | |
| | | CAGCTTCCTTGCGGTATCCGTTCTGCTCACCACAAACGAACAACC | |
| | | CCAACTGTACCTTCCAGGTACTGCGCAAAAATTACCACAAAGTAAT | |
| | | AGGTACTTCCTCAACAAACAGTGAGGACGTGACCCCCTTTGAAAA | |
| | | CTGGCTATATAATACAGCCTCACACTATCAAACTTTTGCCACCGAG | |
| | | GCACAAGTTGGTAGAATACCAAGCTTTAACCCAGACGGTACAAAAA | |
| | | ATACAAAAGAATCTGAATGGCAAAATTACTGGTCCAAAAAAGGTGA | |
| | | ACCATGGAACCCTAATAGTAGTTACCCACATACAACTACAAATCAA | |
| | | ATGTACAAAATACCTTTTGACAGCAACTATGGCTTTCCAACTTACAA | |
| | | ACCAATAAAAGAATACATGTTACAAAGAAGAGCATGGAGTTTCAAA | |
| | | TATGAAACAGACAACCCAGTTAGCAAAAAGATCTGGCCACAACCTA | |
| | | CCACAACAAAACCAACAATAGACTACTATGAATACCACGCAGGCTG | |
| | | GTTCAGTAACATCTTCATAGGCCCCAACAGACACAGCTTACAATTC | |
| | | CAAACAGCATACGTAGACACCACATACAACCCACTGAATGACAAA | |
| | | GGAAAGGGCAACAAGATATGGTTTCAGTATCACAGCAAAGTAAAC | |
| | | ACAGACCTCAGAGACAGAGGCATCTACTGCCTCCTAGAAGACATG | |
| | | CCCCTGTGGTCTATGACCTTTGGATACAGTGACTATGTCAGCACAC | |
| | | AGCTAGGCCCAAACGTGGACCACGAGACTCAAGGCCTTGTGTGCA | |
| | | TAATATGCCCGTACACTGAGCCCCCAATGTATGACAAGACCAATCC | |
| | | AAACAGTGGCTATGTAGCATATGACACAAACTTTGGAAATGGCAAG | |
| | | ATGCCGTCAGGCAGAAGCCAGGTACCCGTGTACTGGCAGTGCAG | |
| | | ATGGAGGCCCATGTTGTGGTTCCAGCAGCAAGTACTGAATGACAT | |
| | | CTCAAAAAGTGGACCGTACGCATACAGAGACGAACTGAAAAACTG | |
| | | TTGCCTGACTGCTTACTACAACTTCATTTTTGACTGGGGGGGCGAC | |
| | | ATGTATTACCCGCAGGTCATTAAAAACCCCTGCGCAGACAGCGGA | |
| | | CTCGTACCCGGTACCAGTAGATTCACTCGAGAAGTACAAGTCGTTA | |
| | | GCCCGCTGTCCATGGGCCCCCAGTACATCCTCCATCTCTTCGACC | |
| | | AAAGACGCGGGTTCTTTAGTTCAAACGCTCTTAAAAGAATGCAACA | |
| | | ACAACAAGAATTTGATGAGTCTTTTACAGTCAAACCTAAGCGACCC | |
| | | AAACTTTCTACAGCCGCCCACGTCGAGCAGCAAGAAGAAGACTCG | |
| | | AGTTCAAGGGAAAGAAAATCGGGGTCCTCACAAGAAGAAGTCCAG | |
| | | GAAGAAGTCCTCCAGACGCCGGAGATCCAGCTTCACCTCCAGCGA | |
| | | AACATCAGAGAACAGCTGCACATCAAGCAGCAGCTCCAACTCCTG | |
| | | TTACTCCAATTATTCAAAACACAAGCAAATATCCACCTGAACCCAC | |
| | | GTTTTATAAGCCCATAA | |
| AAK11698.1 | AF345522.1 | ATGGCGTGGCGCCGGTGGCGATGGCGGCCGTGGTGGAGACGCC | 178 |
| | | GGAGGCGCCGCCGGTGGAGAAGGAGACGGAGGAGACCCAGACG | |
| | | ACGCCGCCCTTATCGACGCCGTCGACCTCGCAGAGTAAGGAGGC | |
| | | GCAGGGGGCGGTGGAGGCGCGCGTACAGACGTTGGGGGCGACG | |
| | | CAGACGCAGACGCAGGCACAAAAAGAAACTTGTACTGACTCAGTG | |
| | | GCAACCAGCAGTAGTTAAGAGGTGCCTAATAGTGGGCTTTGACCC | |
| | | CCTTATAATATGTGGCATTAACAGAACAATATTTAACTACACTACAC | |
| | | ACTCTGAAGACTTTACTTTTAACAACGACAGCTTTGGAGGGGGGCT | |
| | | CTGTACCGCTCAGTACACACTAAGAATCCTTTTCCAAGAAAAGCTG | |
| | | GCCCAGCACAACTTCTGGTCAGCTAGCAACGAAGACCTAGACCTT | |
| | | GCCAGGTACCTAGGAGCCACAATAGTACTTTACAGACACCCTACA | |
| | | GTAGACTTCTTAGTTAGAATTCGCACCAGTCCTCCCTTTGAGGACA | |
| | | CAGACATGACAGCCATGACACTACATCCAGGCATGATGATGCTAG | |
| | | CTAAAAAGACAATTAAAATTCCCAGTCTTAAAACAAGACCGTCCAG | |
| | | AAAACACGTAGTAAGGATTAGAGTAGGGGCCCCTAAACTATTTGAA | |
| | | GACAAGTGGTACCCCCAGAACGAGCTATGTGATGTAACTCTGCTA | |
| | | ACCATACAGGCAACCACAGCTGATTTCCAATATCCGTTCGGCTCAC | |
| | | CACTAACGAACTCCCCCTGTTGCAACTTCCAGGTTCTTAACAGTAA | |
| | | CTATGACAATGCACATTCCATACTTAACTTGTCAAACGAACCAACA | |
| | | AACAAATGGCACACCTATAGAAATAACTGCTATAAATTTCTACTAGA | |
| | | ACAGTACAGCTACTACAACACTAAACAAGTAGTAGCACAACTTAAA | |
| | | TATAAATGGAACCCTAATCAAAACCCTACTATGCCAAATACAAGCA | |
| | | ATGCATCACTTTCTAAAAAACCTGATGACCTTACTAAAACCAAAACA | |
| | | ACAAACGAGTATCCACATTGGGACACCCTATATGGTGGTTTAGCAT | |
| | | ATGGACACAGCACTGTAACACCTGGCACTACCTCATCACCAACAG | |
| | | ACCTAAAAACACAAATGCTTACAGGCAACGAATTTTATACAACAGC | |
| | | AGGCAAAAAGTTAATAGATACATTTCACCCAATTCCTTACTATGAAA | |
| | | ACGGATCTTCTAAAGCCAACACCAACATATTTGACTACTACACAGG | |
| | | CATGTACAGTAGTATTTTCCTGTCTTCAGGCAGATCAAACCCAGAA | |
| | | GTAAAGGGCAGCTACACAGACATCTCTTACAACCCTCTGACAGAC | |
| | | AAGGGAGTAGGTAACATGATTTGGATAGACTGGCTCACTAAAGGA | |
| | | GACACAGTATACGACCCCAAAAAAAGCAAGTGCCTACTCTCAGACT | |
| | | TTCCATTGTGGTCACTTTGTTATGGATACCCAGACTACTGCAGAAA | |
| | | ACAAACCGGAGACTCAGGTATTTACTATGACTACAGAGTACTTATA | |
| | | AGATGTCCATACACATACCCTCAATTAATAAAACACAACGACAAAT | |
| | | ACTTTGGCTTCGTAGTGTACAGCGAAAACTTTGGACTGGGGCGAC | |
| | | TACCAGGAGGCAACCCTAACCCCCCAACTAGAATGAGACTGCACT | |
| | | GGTACCCTAATATGTTCCACCAAACAGAAGTACTAGAGTGCATAGC | |
| | | TCAAAGCGGACCGTTTGCTTATCATGGAGACGAGAGAAAAGCTGT | |
| | | TCTGACTGCCAAATACAAGTTCAGATGGAAGTGGGGAGGCAATCC | |
| | | TGTGTTTCAACAGGTTCTCCGAGACCCCTGCACCGGAGGTGCCGT | |
| | | GGCGCCCCACACCAGTCGACACCCTCGTGCAATACAAGTCCATGA | |
| | | CCCGAAGTATCAGGCCCCGGAGTACCTCTTCCACAAATGGGACTT | |
| | | CAGAAGGGGACTGTTTAGCACTAAAGGTATTAAGAGAGTGTCAGA | |
| | | ACAACCAGTACATGATGAGTATTTTACAGGGAGCAGCAAGAGACC | |
| | | CAAGAAAGACACCAACCCAAGCCCCCAAGGAGAAGAGCAAAAAGA | |
| | | AGGCTCGCGTTTCAGAGTCCCAGAGCTCAGACCCTGGCTCCCCTC | |
| | | CAGCCAGGAAACGCAGAGCCAAAGCGAGCAAGAAGAAACAGCCC | |
| | | CGAAAACGGTCCAAGAGCAGCTACAAGAACAACTCCAGCAGCAGC | |
| | | AGCTCATGGGAATCCAGCTCAGAAACGTCTGTCTCCAGCTCGCAA | |
| | | GAGTCCAAGCGGGGCACAGTCTCCACCCCGTTTTCCAATGCCATG | |
| | | CATAA | |
| AAK11704.1 | AF345525.1 | ATGGCATGGGGATGGTGGAGACGAAGGCGCAAGTGGTGGTGGAG | 179 |
| | | ACGCCGGTTCGCCCGAAGCAGACTTCGCAGACGACGGATTAGAC | |
| | | GCCCTCGTCGCCGCACTCGACGAAGAACAGTAAGGAGGCGCAGA | |
| | | CAATGGAGGAGGGGGCGACCCAGACGCAGACTGTTTAAGAGAAA | |
| | | GAGACGCTTTAAGAGACGCAGACGAAAAGCTAAGATAAAAATAACT | |
| | | CAGTGGCAGCCTAGCTCAGTGAAGAGATGTTTTGTTATAGGATACT | |
| | | TTCCATTAGTAATATGTGGACCCGGAAGGTGGTCAGAAAACTTTAC | |
| | | TAGTCACATAGAAGACAAAATAAGCAAAGGACCCTTTGGGGGAGG | |
| | | GCATAGTACTAGCAGATGGTCCTTAAAAGTACTGTACGAAGAGTTC | |
| | | CAAAGACACCACAACTTTTGGACAAGAAGCAACAAAGACCTAGAG | |
| | | TTAGTTAGATTCTTTGGAAGTAGTTGGAGATTTTACAGACACGAGG | |
| | | ACACTGACTATATAGTGTACTACTCTAGAAAGGCTCCCCTTGGAGG | |
| | | TAACCTTCTAACAGCACCCAGCCTACACCCAGGAGCAGCCATGCT | |
| | | TAGCAAACACAAAATAGTAGTACCCAGTTTTAAAACCAGACCCGGT | |
| | | GGAAAACCCACCGTTAAAATTAATATTAAACCCCCTACAACACTAAT | |
| | | AGACAAATGGTACTTCCAGAAAGACATTTGTGACACAACCTTCCTT | |
| | | AACTTGAACGTTGTACTCTGCAACCTGCGGTTTCCGTTCTGCTCAC | |
| | | CACAAACTGACAACATTTGTGTAACCTTCCAGATATTGCATGAGGT | |
| | | TTACCACAATTACATAAGCATAACTGCAAAAGAGTTACTTACAGGC | |
| | | ACAGAATGGAGACAGTACTACAAAAACTTTTTAAACGCAGCACTAC | |
| | | CAAATGACAGATCTGTAAATAAATTAAACACTTTTAGCACAGAAGG | |
| | | AGCCTACAGCCACCCACAAATAAAAAAACATACAGAAAATATAACA | |
| | | GGTTCAGGAGACAAATACTTTAGAAAAAAAGATGGACTGTGGGGA | |
| | | GATGCTATTCACATTACAGACCAACAAAACAGAACAGAAGTTATAG | |
| | | ACTTAATATTAAAAAATGCAGAAAACTACCTCAAAAAAGTACAACAG | |
| | | GAATACCAAGGACAGGAAAATTTAAAAAACCTTATACATCCCGTCT | |
| | | TTTGTCAGTACGTAGGCATATTTGGGCAGCCCACTACTAAACTACC | |
| | | ACAGAATAAGCCCAGAAATTCCAGGCCTGTACAAAGACATAATATA | |
| | | TAA | |
| AAK11708.1 | AF345527.1 | ATGTCCTGGTGGGGATGGCGCCGCCGATGGTGGTGGAAGCCACG | 180 |
| | | GAGGCGATGGAGACGCAGGAGGGCGCGCCGCCCGAGACGACTA | |
| | | CCGCGACGACGATATAGAAGACCTACTCGCCGCTATCGAGGCAGA | |
| | | CGAGTAAGGAGGCGCCGCGCGGGGGGCTGGCGGGGGCGACGCA | |
| | | GATACTCCCGACGCTATAGCAGACGACTGACTGTCAGACGAAAGA | |
| | | AAAAGAAACTAACTCTTAAGATCTGGCAGCCACAGAATATCAGGAG | |
| | | ATGTAAGATAAGGGGTCTACTGCCCCTCCTGATATGCGGACACAC | |
| | | CCGATCTGCCTTTAACTATGCCATCCACTCGGATGACAAGACCCC | |
| | | CCAACAGCAGAGTTTCGGGGGTGGGCTCAGCACCGTTAGCTTCTC | |
| | | CCTGAAAGTCCTATTCGACCCGAACCAGAGGGGACTTAACAGGTG | |
| | | GTCGGCCAGCAACGACCAGCTTGACCTCGCCCGGTACACGGGCT | |
| | | GCACGTTCTGGTTCTACAGACACAAAAAGACTGACTTTATAGTGCA | |
| | | GTATGATGTCAGCGCCCCCTTCAAACTAGACAAAAACAGTTGTCCC | |
| | | AGCTACCACCCCTTCATGCTCATGAAGGCCAAACACAAGGTCCTC | |
| | | ATCCCCAGTTTTGACACTAAACCCAAAGGCAGAGAAAAGATAAAAC | |
| | | TAAGGATACAGCCCCCCAAGATGTTCATAGATAAGTGGTACACTCA | |
| | | GGAGGACCTATGCCCCGTTATTCTTGTGACACTTGTGGCGACCGC | |
| | | AGCTTCCTTTACACATCCGTTCTGCTCACCACAAACTGCCAACCCT | |
| | | TGCATCACCTTCCAGGTTTTGAAAGAATTCTATTACCAAGCCATGG | |
| | | GGTACGGCACACCAGAAACCACAATGAGCACAATATGGAACACCC | |
| | | TCTACACAACTAGCACCTACTGGCAGTCACACTTAACCCCACAGTT | |
| | | TGTCAGAATGCCCAAAAACAATCCTGATAACACTGCGAACACTGAG | |
| | | GCCAATAAGTTTAATGAGTGGGTTGACAAAACGTTTAAAACAGGCA | |
| | | AGTTAGTTAAATACAACTATAACCAGTATAAACCTGACATAGAGAAA | |
| | | CTAACCCTACTAAGACAATACTACTTTCGATGGGAGACACAGCATA | |
| | | CAGGGGTCGCAGTCCCACCTACGTGGACTACCCCCACAACAGACA | |
| | | GATACGAGTACCACGTAGGCATGTTCAGTCCCATCTTCCTCACCC | |
| | | CTTATAGATCAGCGGGCCTAGACTTTCCGTACGCCTACGCAGACG | |
| | | TCACATACAATCCCCTCACAGACAAAGGGGTGGGCAACCGCATGT | |
| | | GGTACCAGTACAACACTAAGATAGACACCCAGTTCGACGCCAAAT | |
| | | GCTGTAAGTGCGTCCTAGAGGACATGCCCCTCTATGCCATGGCCT | |
| | | TCGGCCACGCAGACTTTCTAGAACAGGAGATAGGAGAGTACCAGG | |
| | | ACCTAGAGGCCAACGGATACGTGTGTGTTATCAGTCCCTACACCA | |
| | | AGCCCCCCATGTTCAACAAACACAACCCTCAGCAGGGATACGTGT | |
| | | TCTATGACTCACAGTGGGGCAATGGCAAATGGATAGACGGCACCG | |
| | | GGTTCGTCCCAGTGTACTGGCTGACCAGATGGAGAGTAGAACTGC | |
| | | TATTTCAAAAGCAAGTACTCTCAGACCTCGCCATGTCAGGGCCCTT | |
| | | CAGCTATCCAGACGAACTTAAGAACACAGTACTGACGGCCAAGTA | |
| | | CAGATTTGACTTTAAGTGGGGTGGCAATCTCTTCCACCAACAGACC | |
| | | ATTAGAAACCCCTGCAAACCCGAAGAGACCTCGACCGGTAGAATC | |
| | | CCTCGCGATGTACAAGTCGTTGACCCGGTCACCATGGGCCCCCGA | |
| | | TTCGTCTTTCACTCCTGGGACTGGAGGAGAGGGTTCCTTAGTGAC | |
| | | AGAGCTCTCAAAAGAATGTTTGAGAAACCGCTCGATTTTGAGGGAT | |
| | | TTACAGCGACTCCAAAACGACCTCGCATACTCCCTCCCACAGAGG | |
| | | GACAGCTCGCCCGAGAGCAAAAAGAGCAAGAAGAAAGCTCAGATT | |
| | | CGCAGGAAGAAAGCAGCCTTACCCCGCTCGAAGAAGTCCCGCAA | |
| | | GAGACGAAGCTACGACTCCACCTCAGAAAGCAGCTCCGAGAGCA | |
| | | GCGAAGCATCAGACACCAACTCAGAACCATGTTCCAGCAGCTTGT | |
| | | CAAGACGCAAGCGGGCCTACACCTAAACCCCCTTTTATCTTCCCA | |
| | | GCTGTAA | |
| AAK11710.1 | AF345528.1 | ATGTGGAATCCATCCACAATTAGAGCATGTAACATAAAGGGTGCTA | 181 |
| | | TAAACCTTGTAATGTGCGGACACACTCAGGCAGGCAGAAACTATG | |
| | | CCATTAGAAGTGAAGACTTTTATCCTCAAATACAAAGCTTTGGTGG | |
| | | GTCATTTAGTACAACTACATGGAGCCTTAGAGTACTGTTTGATGAA | |
| | | TACCAAAAGTTCCACAACTTTTGGACATATCCTAATACTCAGCTAGA | |
| | | TCTATGTAGATATAAATATGCTATATTTACCTTTTACAGAGACCCTA | |
| | | AAGTAGACTACATTGTTATATACAACACAAATCCACCATTTAAAATT | |
| | | AACAAATACAGTAGTCCCTTTTTACACCCCGGACTTATGATGTTAC | |
| | | AAAAAAAAAAAATACTAATACCTAGCTTTCAAACAAAACCAGGGGG | |
| | | CAAATCTAGAATTAAGGTTAAAATTAAGCCCCCTGCTCTATTTGAAG | |
| | | ACAAGTGGTACACTCAACAAGACTTGTGTCCAGTAAACCTGTTGTC | |
| | | ACTTGCGGTTTCCGCCTGCAGCTTTATACATCCGTTCTGCTCACCA | |
| | | GAAAGTGACACAATATGCATGACATTTCAGGTATTGCGAGAGTTTT | |
| | | ACTACACACACCTAACTGTCACTCCAACCACAACTACCTCCACACC | |
| | | AGAAAAAGACAAAAAAATATTTAATGACCAATTATACTCCAACGCTA | |
| | | ACTTTTATCAATCGCTACACGCATCAGCGTTCTTAAACATTGCTCA | |
| | | GGCACCTGCTATACATGGCCACAATGGAATACCAAACAACAGTAG | |
| | | GTATTTAAGTTCCACAGGTACAGAAACAAGTTTTAGAACTGGAAAC | |
| | | AATAGTATATATGGACAACCAAATTATAAACCAATTCCAGAGAAATT | |
| | | AACAGAAATAAGAAAGTGGTTTTTCAAACAAGCTACAACACCTAAT | |
| | | GAAATTCATGGCACATATGGAAAACCAACATATGATGCAGTAGACT | |
| | | ACCACTTAGGCAAATACAGTCCAATATTCTTAAGTCCATACAGAAC | |
| | | TAACACACAATTTCCCACTGCATACATGGATGTAACTTATAATCCAA | |
| | | ATGTAGATAAAGGAAAAGGCAACAAAATATGGCTTCAATCAGTAAC | |
| | | AAAAGAAACATCTGATTTTGACTCACGTAGCTGCAGATGTATAATA | |
| | | GAAAACTTACCCATGTGGGCCATGGTTAACGGGTACTCAGACTTT | |
| | | GCAGAGTCTGAATTAGGATCTGAAGTACACGCTGTATATGTTTGCT | |
| | | GTATTATTTGTCCTTACACAAAACCTATGCTATATAACAAAACAAAC | |
| | | CCAGCAATGGGCTATATATTTTATGATACTTTATTTGGCGACGGAA | |
| | | AACTACCATCAGGTCCAGGTCTTGTTCCATTTTATTGGCAAAGCAG | |
| | | ATGGTATCCAAAACTAGCTTGGCAACAACAAGTACTACATGATTTTT | |
| | | ATTTGTGTGGCCCCTTTAGCTACAAAGATGACCTCAAAAGCTTTAC | |
| | | TATAAACACAACTTACAAGTTTAAATTCTTATGGGGTGGAAATATGA | |
| | | TTCCCGAACAGGTTATCAAAAACCCGTGCAAAACAACAGATCCAAC | |
| | | ATACACCCTGTCCGATAGACAGCGTCGCGACCTACAAGTTGTTGA | |
| | | CCCAATTACCATGGGCCCGCAGTGGGAATTCCACACCTGGGACTG | |
| | | GCGACGCGGACTGTTTGGACAAAATGCTCTTAGAAGAGTGTCAGA | |
| | | AAAACCAGGAGATGATGCAGAGTATTATGCGCCTCCAAAAAAACCT | |
| | | AGATTTTTCCCACCAACAGACCTCGAAGAGCAAGAAAAAGACTCAG | |
| | | ATTCACAGGAGGAGACGAGACTCCTATTCCACCCGTCGCCGCCAA | |
| | | GGAGCCAAGAAGAGATCCAGCAAGAGCAGCAGCGAGACATCCAC | |
| | | CTCAGACTCGGACAACAACTCAGAATCAGACAGCAGCTCCAGCAA | |
| | | GTGTTCTTACAAGTCCTCAAAACGCAAGCGAACCTCCACATAAATC | |
| | | CATTATTCTTAAACCAACAATAA | |
| AAK11712.1 | AF345529.1 | ATGGCATGGGGATGGTGGAGACGGTGGCGCCGGTGGCCCACCA | 182 |
| | | GACGCTGGAGGAGACGCCGTCGCCGGCGCCCCGTACGGAGAAC | |
| | | AAGAGCTCGCCGACCTGCTCGACGCTATAGAAGACGACGAACAGT | |
| | | AAGAACCAGGCGGAGGCGGTGGGGGCGCAGACGGTACAGACGG | |
| | | GGCTGGAGACGAAGGACTTATGTAAGGAAGGGGCGACACAGAAA | |
| | | AAAGAAAAAGAGACTCGTACTGAGACAGTGGCAGCCAGCCACCAG | |
| | | ACGCAGATGCACTATAACTGGGTACCTGCCCATAGTGTTCTGCGG | |
| | | ACACACTAAGGGCAATAAAAACTATGCACTACACTCTGACGACTAC | |
| | | ACCCCCCAAGGACAGCCATTTGGAGGGGCCCTTAGCACTACCTCT | |
| | | TTCTCCCTAAAAGTGTTGTATGACCAGCACCAGAGGGGACTAAACA | |
| | | AGTGGTCTTTTCCCAACGACCAGCTAGACCTTGCCAGATACAGAG | |
| | | GCTGCAAATTCTACTTCTATAGAACCAAACAGACTGACTGGGTGGG | |
| | | CCAGTATGACATATCAGAACCCTACAAGCTAGACAAGTACAGCTGC | |
| | | CCTAACTACCACCCGGGAAACATGATTAAGGCAAAGCACAAATTTT | |
| | | TAATTCCAAGCTATGATACTAATCCCAGAGGGAGACAAAAAATTAT | |
| | | AGTTAAAATTCCCCCCCCAGACCTTTTTGTAGACAAGTGGTACACT | |
| | | CAGGAAGACCTGTGTGACGTTAATCTTGTGTCATTTGCGGTTTCTG | |
| | | CGGCTTCCTTTCTCCACCCATTCGGCTCACCACAAACTGACAACCC | |
| | | TTGCTACACCTTCCAGGTGTTGAAAGAATTCTACTATCAGGCAATA | |
| | | GGCTTTAGTGCAACAGAGGAAAAAATACAAAATGTTTTTAACATATT | |
| | | ATACGAAAACAACTCATACTGGGAATCAAACATAACTCCCTTTTATG | |
| | | TAATTAATGTTAAAAAAGGGTCTAACACAGCACAGTACATGTCACC | |
| | | TCAAATTTCAGACGCAGATTTTAGAAATAAAGTAAATACTAACTACA | |
| | | ACTGGTATACCTACAATGCCAAAACCCATAAAGAAAAATTAAAAAC | |
| | | GCTAAGACAAGCATACTTTAAACAATTAACCTCTGAAGGTCCGCAA | |
| | | CACACATCCTCTCACGCAGGCTACGCCACTCAGTGGACCACCCCC | |
| | | AGCACAGACGCCTACGAATACCACCTAGGCATGTTTAGTACCATCT | |
| | | TTCTAGCCCCAGACAGACCAGTACCTCGCTTTCCCTGCGCCTACC | |
| | | AAGATGTCACCTACAATGCCTTAATGGACAAAGGGGTGGGCAACC | |
| | | ACGTGTGGTTTCAGTACAACACAAAGGCAGACACTCAACTAATACT | |
| | | CACCGGAGGGTCCTGCAAAGCACACATAGAAAACATACCCCTGTG | |
| | | GGCAGCCTTCTATGGCTACAGCGACTTCATAGAGTCAGAGCTAGG | |
| | | CCCCTTTGTAGACGCAGAGACAGTAGGCCTTATATGTGTAATCTGC | |
| | | CCCTACACTAAACCCCCCATGTACAACAAGACAAATCCCATGATGG | |
| | | GGTACGTGTTTTATGACAGAAATTTTGGTGACGGCAAATGGACTGA | |
| | | CGGACGGGGCAAAATAGAGCCCTACTGGCAGGTTAGGTGGAGGC | |
| | | CAGAAATGCTTTTTCAAGAGACTGTAATGGCAGACATAGTTCAAAC | |
| | | CGGGCCCTTTAGCTACAAGGACGAACTTAAAAACAGCACACTAGT | |
| | | GTGCAAATACAAATTCTATTTCACCTGGGGAGGTAACGTGATGTTC | |
| | | CAACAGACGATCAAAAACCCATGCAAGACGGACGAACAACCCACC | |
| | | GACTCCGGTAGACACCCTAGAGGAATACAAGTGGCGGACCCGGA | |
| | | ACAAATGGGACCCCGTTGGGTGTTCCACTCCTTTGACTGGCGAAG | |
| | | GGGCTATCTTAGCGAGAAAGCTCTCAAACGCCTGCAAGAAAAACC | |
| | | TCTTGACTATGACGAATATTTTACACAACCAAAAAGACCTAGAATGT | |
| | | TTCCTCCAACAGAATCAGCAGAAGGAGAGTTCCGAGAGCCCGAAA | |
| | | AAGGCTCGTATTCAGAGGAAGAAAGGTCGCAAGCCTCTGCCGAAG | |
| | | AGCAGACGAAAGAGGCGACAGTACTTCTCCTTAAACGACGACTCA | |
| | | GAGAGCAACAGCAGCTCCAGCAGCAGCTCCAATTTCTCACCCGAG | |
| | | AAATGTTCAAAACGCAAGCGGGTCTCCACCTAAACCCTATGTTATT | |
| | | AAACCAGCGGTGA | |
| AAK54731.1 | AF371370.1 | ATGCGCTTTTCCAGAATCTACAGGCCAAAGAAAGGGCCACTGCCA | 183 |
| | | CTGCCTCTGGTGCGAGCAGAACAGAAAAAACAGCCTAGTGATATG | |
| | | AGTTGGCGCCCTCCGCTTCACAATGGGGCAGGAATCGAGCGTCA | |
| | | GTTTTTCGAAGGCTGCTTTCGATTCCACGCTAGTTGTTGCGGCTGT | |
| | | GGCAATTTTGTTACTCATATTACTCTACTGGCTGCTCGCTATGGTTT | |
| | | TACTGGGGGGCCGACGCCGCCAGGTGGTCCTGGGGCGCTACCCT | |
| | | CGCTAAGGAGAGCGCTGCCACCTCCTCCGGCCCCCCAAGACCAG | |
| | | GCTGAACCAGAGCTATGGCGTGGTCGTGGTGGTGGAGGCGAAGG | |
| | | AAACGCTGGTGGCCGCGCAGAAGGAGGCGATGGAGAAGGCTACG | |
| | | AACCCGAAGAACTGGAAGAGCTGTTCCGCGCCGCCGCCGCCGAC | |
| | | GACGAGTAA | |
| BAB69916.1 | AB060596.1 | ATGGCGTTCCGGTGGTGGTGGTGGAGACGCCGCCCGCAGCGACG | 184 |
| | | ATGGACCCGGCGCCGATGGAGGAGACTACGAACCCGCCGACCTA | |
| | | GACGCACTGTACGACGCCGTCGCCGCAGACCAAGAGTAAGGAGA | |
| | | AGGCGGTGGGGCAGGAGACGTGGGCGACGCAGACTGTACAGAC | |
| | | GCACATATAGAAAAAGGCGCAAAAGACGAAAAAAAATGACCTTAAA | |
| | | AATGTGGAATCCATCCACAATTCGCGCCTGTAACATTAGGGGCTTC | |
| | | ATAGCACTAGTAGTCTGTGGACACACTCGTGCAGGCTGTAACTAT | |
| | | GCCATACACAGCGAAGACTACATACCTCAACTAAGACCCTACGGA | |
| | | GGGTCTTTCAGCACTACTACTTGGAGTCTAAAACTACTATTTGACG | |
| | | AATATCTGAAATTTAGAAACAAATGGAGCTACCCCAACACAGAACT | |
| | | AAACCTTGCTAGATACAGGGGAGCCACATTTACATTTTACAGAGAC | |
| | | CCCAAAGTAGACTATATAGTAGTATACAACACAGTACCTCCATTTAA | |
| | | ACTTAACAAATACAGCTGCCCCATGCTGCACCCAGGTATGATGATG | |
| | | CAGTACAAAAAGAAAGTTTTAATACCAAGCTATCAGACAAAACCAA | |
| | | AGGGAAAAGCCAAAATAAGACTTAGAATAAAACCTCCAGTTTTATTT | |
| | | GAAGACAAATGGTACACCCAGCAAGACCTGTGTCCCGTTAATCTTT | |
| | | TGTCACTTGCGGTTAGCGCATGTTCCTTCCTGCATCCGTTTATACC | |
| | | ACCAGAAAGTGACAACATATGCATAACGTTCCAGGTGTTGCGAGA | |
| | | CTTTTATTACACACAAATGTCAGTTACACCCACAACAACCACTTCCC | |
| | | TAAATCAGAAAGATGAAAAAATATTTAGTGACCACTTATATAAAAAC | |
| | | CCTGAATACTGGCAATCACATCACACAGCTGCTAGACTATCTACCT | |
| | | CTCAAAAACCTGCACTACGAAATAAAGAAGAAATACCTAATGATCA | |
| | | CGGATACTTAAACACAACACCAACTGACAGTACTTTTAGAACTGGA | |
| | | AACAATACAATATATGGCCAACCAAGCTACAGACCAAACTATACCA | |
| | | AACTAACTAAGATTAGAGAATGGTACTTTACACAAGAAAACACAGA | |
| | | CAACCCAATACATGGCAGCTACTTAAAACCAACACTAAACTCTGTA | |
| | | GACTACCACCTAGGAAAATACAGTGCTATATTCTTAAGTCCCTATA | |
| | | GAACAAACACTCAATTTGATACAGCATACCAAGATGTAACCTACAA | |
| | | TCCTAACACAGACAAAGGCAAAGGCAATAAAATATGGATTCAGAGC | |
| | | TGTACAAAAGAATCCACCATACTAGACAACGCATGCAGATGTGTAA | |
| | | TAGAAGACATGCCATTATGGGCTATGGTAAATGGCTACTTAGAATT | |
| | | CTGTGACTCAGAGCTTCCAGGAGCCAACATCTACAATACATACATA | |
| | | GTAGTTGTTATATGCCCTTACACCAAACCTCAACTACTAAACAAAAC | |
| | | TAATCCAAAACAAGGCTATGTATTTTATGACACTCTATTTGGAGACG | |
| | | GAAAAATGCCCACAGGAACAGGCCTAGTACCGTTCTGGCTGCAGA | |
| | | GCAGATGGTACCCCAGAGCAGAGTTCCAACAACAAGTACTACATG | |
| | | ACCTTTACCTTACAGGCCCATTTAGCTACAAAGATGACCTAAAATC | |
| | | CTTTAGCTTTAATGCTAAATACAAATTCTCATTCTTATGGGGCGGCA | |
| | | ATATGATTCCCCAACAGATTATCAAAAACCCGTGTAAAAAAGAAGA | |
| | | ATCCACATTCACCTATCCCAGTAGAGAGCCTCGCGACCTACAAGTT | |
| | | GTTGACCCACTCACCATGGGCCCAGAATGGGTCTTCCACACATGG | |
| | | GACTGGAGACGTGGACTTTTTGGTAAAAATGCTGTCGACAGAGTG | |
| | | TCAAAAAAACCAGACGATGATGCAGAATATTATCCAGTACCAAAAA | |
| | | GGCCTCGATTCTTCCCTCCAACAGACACACAGTCAGAGCCAGAAA | |
| | | AAGACTTCGGTTTCACACCGGAGAGCCAAGAGTTACAGCAAGAAG | |
| | | ACTTACGAGCACCCCAAGAAGAAAGCCAAGAGGTACAGCAGCAGC | |
| | | GACTGCTCCAGCTCAGACTCTCACAGCAGTTCAGACTCAGACAGC | |
| | | AGCTCCAGCACCTGTTCGTACAAGTCCTCAAAACCCAAGCAGGTC | |
| | | TCCACATAAACCCATTATTTTTAAACCATGCATAA | |
| BAB69900.1 | AB060592.1 | ATGGCGTGGACCTGGTGGTGGCAGAGGAGGCGCCGAAGGTGGC | 185 |
| | | CGTGGAGAAGGAGAAGGTGGAGAAGACTACGCACCAGAAGACCT | |
| | | AGACGACTTGTTCGCCGCCGTCGCAAGAGATACAGAGTAAGGAGA | |
| | | CGGAGGCGGTGGGGAAGGAGACGTGGGCGACGCACATACCTTAG | |
| | | ACGCAGACTTAAAAAAAGAAAGAGACGCAAAAAGCTAAGACTGAC | |
| | | TCAATGGAACCCTAGCACAATTAGAGGATGTACAATTAAGGGAATG | |
| | | GCTCCCCTAATTATCTGTGGCCACACTATGGCAGGCAATAACTTTG | |
| | | CCATCCGAATGGAGGACTATGTCTCTCAAATTAGACCATTCGGAG | |
| | | GGTCGTTTAGCACCACAACCTGGAGCCTTAAAGTACTTTGGGACG | |
| | | AGCACACCAGATTCCATAACACCTGGAGCTACCCAAACACTCAGC | |
| | | TAGATCTCGCAAGGTTTAAAGGAGTAAACTTTTACTTCTACAGAGA | |
| | | CAAAGACACAGACTTTATAGTAACATACAGCTCAGTCCCGCCATTT | |
| | | AAAATGGACAAATACTCATCAGCCATGCTACATCCAGGCACGCTCA | |
| | | TGCAGAGAAAGAAAAAGATATTAATACCCAGCTTTACAACAAGACC | |
| | | AAGGGGCCGAAAAAAAGTTAAACTGCATATAAAACCTCCTGTTTTA | |
| | | TTTGAAGACAAATGGTACACCCAGCAGGACCTCTGCGACGTTAAT | |
| | | CTTTTGTCACTTGCGGTTTCTGCGGCTTCCTTTAGACATCCGTTCT | |
| | | GCCCACCACAAACTGACAACATTTGCATCACTTTCCAGGTGTTGAA | |
| | | AGACTTCTATTACACACAAATGTCAGTTACACCGGACACAGCAGGC | |
| | | CAAGAAAAAGACATTGAAATATTTGAAAAACACTTATTTAAAAATCC | |
| | | ACAATTCTATCAAACTGTCCACACACAAGGAATAATTAGCAAAACA | |
| | | CGAAGAACAGCTAAATTTTCAACCTCAAATAATACCCTAGGAAGTG | |
| | | ACACGAATATAACGCCATACCTAGAACAACCAACAGCAACAAACCA | |
| | | CAAAAACACATTATCCACAGGTAACAACTCAATATATGGCCTTCCA | |
| | | TCTTACAACCCAATACCAGATAAACTTAAAAAAATTCAAGAATGGTT | |
| | | TTGGAAACAAGAAACTGACAAAGAAAATTTAGTTACTGGCTCCTAT | |
| | | CAAACACCTACTAACAAATCAGTAAGCTACCATCTAGGAAAATACA | |
| | | GCCCCATATTTTTAAGCTCATATAGAACTAATCTACAGTTTATAACT | |
| | | GCATACACAGATGTAACATACAATCCCCTAAATGACAAAGGAAAAG | |
| | | GCAACCAAATATGGGTACAGTATGTAACAAAACCAGATACTATATT | |
| | | TAATGAAAGACAGTGCAAATGCCACATAGTAGATATTCCTTTGTGG | |
| | | GCAGCATTCCATGGCTATATTGACTTTATACAAAGTGAACTAGGCA | |
| | | TACAAGAAGAAATACTAAACATTGCCATAATAGTAGTTATATGTCCA | |
| | | TACACAAAACCCAAACTAGTACACGACCCACCAAACCAAAACCAAG | |
| | | GCTTTGTATTCTATGACACACAATTTGGAGACGGTAAAATGCCAGA | |
| | | GGGCTCGGGCCTAGTACCCATATACTACCAAAACAGATGGTATCC | |
| | | TAGAATAAAGTTCCAGAGTCAAGTAGTGCATGACTTTATACTAACA | |
| | | GGCCCCTTTAGCTACAAAGATGATCTAAAGAGCACAGTACTAACAG | |
| | | TAGAATACAAGTTTAAATTCTTATGGGGCGGCAATATGATTCCCGA | |
| | | ACAGGTTATCAGAAACCCTTGTAAAACAGAAGGACACGATCTCCCT | |
| | | CACACCAGTAGACTCCATCGCGACTTACAAGTTGTTGACCCACACA | |
| | | CCGTGGGCCCCCAATGGGCGCTCCACACCTGGGACTGGCGACGT | |
| | | GGACTCTTTGGTTCAGAGGCTATCAAAAGAGTGTCTGAACAACAAG | |
| | | TACATGATGAACTGTATTACCCAGCTTCAAAGAAACCTCGATTCCT | |
| | | CCCTCCAATATCAGGCCTCCAAGAGCAAGAAAGAGACTACAGTTC | |
| | | GCAGGAGGAAAAAGACCAGTCCTCCTCAGAAGAAGAGAAGGACC | |
| | | CGAAGAAAAAAGAGCAAAAACAGCAGCAGCGACTCCACCTCCAGT | |
| | | TCCAAGAGCAGCAGCGACTCGGAAACCAACTCCGACTCATCTTCC | |
| | | GAGAGCTACAGAAAACCCAAGCGGGTCTCCACATAAATCCTATGTT | |
| | | ATCAAACCGGCTATAA | |
| BAB69904.1 | AB060593.1 | ATGGCCTGGAGATGGTGGTGGAGACGGCGCTGGAAGCCAAGAAG | 186 |
| | | GCGGCCAGCGTGGACCAAGTACCGCAGACGCAGGTGGAGACGAC | |
| | | TTCGACCCCGCAGACCTAGAAGACTTGCTCGCGGCCGTCGAAGAA | |
| | | GACGAACAGTAAGGAGGCGGAGGGTCAGGAGACTCAGACGGAGG | |
| | | AGGGGGTGGACTAGGAGACGGTACTTGAGACGCAGAAAGAGACG | |
| | | AAAGCTAATACTGACTCAGTGGAACCCCAATATTGTCAGACGATGC | |
| | | TCTATAAAGGGTATAATCCCCCTCACAATGTGCGGCGCTAACACC | |
| | | GCCAGTTTTAACTATGGGATGCACAGCGACGACAGCACCCCTCAG | |
| | | CCAGAGAAATTTGGGGGAGGCATGAGCACAGTGACCTTTAGCCTG | |
| | | TATGTACTGTATGACCAGTTCACTAGACACATGAACCGGTGGTCTT | |
| | | ATTCCAACGACCAGCTAGACCTGGCCAGATACAGGGGCTGCTCAT | |
| | | TCAAACTGTACAGAAACCCCACAACTGACTTTATAGTGCAGTATGA | |
| | | CAATAATCCTCCTATGAAAAACACTATACTGAGCTCACCTAACACT | |
| | | CACCCAGGTATGCTCATGCAGCAGAAACACAGGATACTAGTGCCC | |
| | | AGCTGGCAGACCTTTCCCAGGGGGAGAAAATATGTTAAAGTTAAG | |
| | | ATACCCCCACCTAAACTCTTTGAGGACCACTGGTACACTCAGCCA | |
| | | GACTTATGCAAAGTTCCGCTCGTTACTCTGCGGTCAACCGCAGCT | |
| | | GACTTCAGACATCCGTTCTGCTCACCACAAACGAACAACCCTTGCA | |
| | | CCACCTTCCAGGTGTTGCGAGAGAACTATAACGAAGTCCTAGGAC | |
| | | TTCCCTATGCTAACACCGGGTCTAACAATGAAGTCAAAATTAAAATT | |
| | | GATAACTTTGAAAACTGGCTTTATAACTCCAGTGTACACTATCAAAC | |
| | | ATTCCAAACAGAGCAAATGTTCAGACCCAAACAATACAATGCAGAT | |
| | | GGCTCTACCTGGAAAGACTACAAAAGCATGTTATCTACATGGACAT | |
| | | CACAAATATATAACAAGAAAACAGACAGCAACTATGGGTATGCCTC | |
| | | CTATGACTTTAGTAAAGGTAAAGAGTTTGCTACACAAATGAGACAG | |
| | | CATTACTGGGTACAACTAACACAACTAACAGCCACAGTCCCACACA | |
| | | TAGGACCTACTTACAGCAACACAACCACACCAGAATACGAATATCA | |
| | | CGCAGGCTGGTACTCTCCAGTGTTCATAGGCCCCAACAGACACAA | |
| | | CATACAGTTCAGAACAGCATACATGGACGTTACCTACAACCCACTA | |
| | | AATGACAAAGGCCAGTTTAACAGAGTATGGTTCCAGTACAGCACTA | |
| | | AACCCACCACAGACTTCAACAACACACAGTGCAAATGTGTTCTAGA | |
| | | AAACATTCCACTGTGGTCAGCCCTATTTGGATACTCTGAATATGTA | |
| | | GAGAGCCAGCTAGGCCCCTTCCAGGACCACGGGACCGTGGGTGT | |
| | | AGTAGTAGTACAATGTCCTTACACAGTGCCACCCATGTATAACAAA | |
| | | GAGAAACCAGACATGGGCTACGTATTCTATGACACACATTTTGGCA | |
| | | ATGGCAAATTGGGCAACGGCAGCGGCCAGGTACCCAGGTACTGG | |
| | | CAGATGAGATGGTACCCCATACTCAAAAGACAAAAACAAGTAATGA | |
| | | ATGACATTTGCAAGACTGGACCGTTCAGCTACAGAGACGAACTGC | |
| | | TTCAGGTGGACTTAGCAAGCCCCTACACCTTCAGATTTAACTGGG | |
| | | GGGGCGACTTACTCTACCACCAGGTCATCAAAGACCCGTGCAGCT | |
| | | CCTCAGGACTGGCACCTACCGACTCCAGTAGATTCAAGCGGGATG | |
| | | TACAAGTCGTTAGCCCGCTCACAATGGGGCCCCGACTGCTATTCC | |
| | | ACTCGTTCGACCAAAGACGAGGGTTCTTTACTCCAGGAGCTATCAA | |
| | | ACGAATGCATGATGAACAAATTAATGTTCCAGACTTTACACAAAAA | |
| | | CCTAAAATCCCGCGAATTTTCCCACCAGTCGAGCTCCGAGAAAGA | |
| | | GCAGAAGCCGAAGAAGACTCAGGTTCGGAAAAAGCGTCGTTCACC | |
| | | TCGTCGCAAGAGAGAGAAGCCGAAGCCCAAGAAAAGTTACCGATA | |
| | | CAGCTCCAGCTCAGACAGCAGCTCAGACAACAACAGCAGCTCCGA | |
| | | GTCCACTTGCAGCAAGTCTTCCTCCAACTCCAAAAAACGAAGGCA | |
| | | CATTTACATATAAACCCACTATTTTTGGCCCAAGGGAACATGTAA | |
| BAB69912.1 | AB060595.1 | ATGGCCTACTCCTACTGGTGGCGCCGCCGGAGGTGGCCGTGGAG | 187 |
| | | AGGCCGATGGAGGCGCTGGAGGCGCCGCAGACGAATACCGCGC | |
| | | CGAAGACCTAGACGACCTGTTCGCCGCTATCGAAGGAGACCAGTA | |
| | | AGGAGAAAGCGTCGGTGGGGGAGGCGAGGGCGACGGCGCCGGT | |
| | | ACACTAGACGGTACAGACGCAGACTGACTGTCAGACGAAAGAGAA | |
| | | ACAAACTCAGACTGAGCGTATGGCAGCCCCAGAATATCAGATACT | |
| | | GTGCCATAAAAGGCCTCTTTCCCATCCTCATCTGCGGGCACGGAA | |
| | | AGAGCGCCGGCAACTATGCCATCCACTCGGATGACTTTATCACAA | |
| | | GCAGATTCTCTTTCGGAGGTGGTCTCAGCACGACCTCCTACTCTCT | |
| | | GAAGCTGCTATTCGACCAAAACCTCAGGGGACTAAACAGATGGAC | |
| | | CGCTAGCAACGACCAGCTAGACCTAGCTAGGTACCTGGGGGCCAT | |
| | | ATTCTGGTTCTACAGAGACCAGAAAACAGACTACATAGTCCAGTAT | |
| | | GACATCTCAGAGCCCTTCAAGATAGACAAAGACAGCTCCCCTTCCT | |
| | | TCCATCCAGGCATACTGATGAAAAGCAAACACAAAGTACTGGTACC | |
| | | CAGCTTCCAGACTTGGCCCAAGGGTCGCTCTAAAGTAAAGCTAAA | |
| | | GATAAAGCCCCCCAAGATGTTCGTTGACAAATGGTACACACAAGA | |
| | | GGATCTCTGTACCGTTACTCTTGTGTCACTTGTGGTCAGCCTAGCT | |
| | | TCCTTTCAACATCCGTTCTGCCGACCACTAACTGACAACCCTTGCG | |
| | | TCACCTTCCAAGTTCTGCAAAATTTCTACAACAACGTAATAGGCTA | |
| | | CTCCTCATCAGACACACTAGTAGATAATGTCTTTACGAGTCTGTTAT | |
| | | ACTCTAAAGCCTCCTTCTGGCAGAGCCATCTGACCCCCTCTTATGT | |
| | | CAAAAAAATTAACAACAACCCCGATGGCAGCTCAATTAGTCAGCGA | |
| | | GTAGGCACAATGCCTGACATGACGGAGTATAACAAGTGGGTATCC | |
| | | AACACAAATATAGGAACAGGATTCGTAAACTCAAATGTTAGTGTAC | |
| | | ACTATAATTATTGTCAGTACAACCCTAACCATACTCATTTAACAACA | |
| | | CTGAGACAGTACTACTTCTTTTGGGAAACACACCCAGCAGCGGCC | |
| | | AACAAAACACCTGTAACACACGTCCCCATCACCACCACAAAACCCA | |
| | | CCAAAGACTGGTGGGAGTACAGATTAGGCCTGTTCAGTCCCATCT | |
| | | TCCTATCTCCACTCAGAAGCAGCAACATAGAGTGGCCCTTCGCATA | |
| | | CAGAGACATAATATACAACCCACTCATGGACAAGGGGGTAGGTAA | |
| | | CATGATGTGGTACCAGTACAACACAAAACCAGATACCCAGTTCTCC | |
| | | CCCACCTCTTGCAGAGCAGTGCTAGAAGACAAACCCATATGGTCC | |
| | | ATGGCATATGGGTATGCAGACTTTCTGCTGTCCATACTAGGTGAAC | |
| | | ACGACGATGTAGACTTCCATGGATTAGTCTGTATCATATGCCCCTA | |
| | | CACCAGACCGCCCCTCTTCGACAAGGATAACCCCAAGATGGGCTA | |
| | | TGTCTTCTACGATGCTAAATTTGGCAATGGCAAATGGATAGACGGT | |
| | | ACGGGATTCATCCCGGTAGAGTTCCAGAGTAGATGGAAACCAGAG | |
| | | CTGGCCTTCCGGAAAGACGTACTGACTGACTTAGCCATGTCAGGC | |
| | | CCCTTCTCCTACAGCGACGACCTTAAAAACACCACAATCCAGGCC | |
| | | AAGTACAAATTCAAATTCAAATGGGGCGGTAATCTCTCTTACCACC | |
| | | AGACGATCAGAAACCCGTGCACCTCGGACGGACAGACGCCCACA | |
| | | ACCAGTAGACAGTCTAGAGAGGTACAAATCGTTGACCCGCTCACC | |
| | | ATGGGACCCCGATACGTATTCCACTCGTGGGACTGGCGACGTGG | |
| | | GTGGCTTAATGACAGAACTCTCAAACGCTTGTTCCAAAAACCGCTC | |
| | | GATTTTGAAGAGTATCCAAAATCTCCAAAGAGACCTAGAATTTTCC | |
| | | CACCCACAGAGCAGCTCCAAGAAGACCCGCAAGAGCAAGAAAGA | |
| | | GACTCCTCTTCTTCGGAAGAAAGTCTCCCTACATCGTCAGAAGAGA | |
| | | CACCGCCAGCCCACCTACTCAGAGTACACCTCAGAAAGCAGCTCC | |
| | | GGCAACAGCGAGACCTCCGAGTCCAGCTCAGAGCCCTGTTCGCC | |
| | | CAAGTCCTCAAAACGCAAGCGGGCCTACACATAAACCCCCTCTTAT | |
| | | TGGCCCCGCAGTAA | |
| BAB79314.1 | AB064596.1 | ACGGCCTGGTGGTGGGGAAGACGGTGGCGACGCCGCCCGTGGG | 188 |
| | | GCCGCTGGCGCCGCCGAAGGCGCGTATGGAGAAGAAGACCTAGA | |
| | | ACTGCTGTTCGCCGCCGCCGAGGAAGACGATATGTGAGTAGAAG | |
| | | GCGCCGCTACAGGCGCAGACTCAGACGAAGGGGCAGACGGAGAT | |
| | | ACAGGGGGCGACGAAAGAAGAGACAGACCCTAGTACTCAAACAAT | |
| | | GGCAACCCGACGTTAACAGACTGTGCAGAATCACAGGATGGCTAC | |
| | | CTCTTATAGTTTGTGGCACCGGCAGGGCCCAGGACAACTTTATAG | |
| | | TACACTCAGAGGACATAACCCCCCGAGGAGCCGCCTACGGGGGC | |
| | | AACCTCACACACATAACATGGTGCTTAGAAGCTATATACCAAGAAT | |
| | | TCCTCATGCACAGAAACAGATGGTCCAGAAGTAACCATGACCTGG | |
| | | ACCTCTGCAGATACCAAGGAGTAGTTTTTAAGGCCTATAGACACCC | |
| | | CAAAGTTGACTACATACTAGCATACACAAGAACACCTCCATTTCAA | |
| | | GCAACAGAACTTAGCTACATGTCCTGCCATCCACTACTCATGCTGA | |
| | | CAGCAAAACACAGGATAGTAGTAAAGAGCCAAGAGACCAAAAAAG | |
| | | GGGGCAAAAAATATGTAAAATTTAGAATAAAGCCCCCCAGACTAAT | |
| | | GTTAAACAAGTGGTACTTCACTCATGACTTTTGTAAAGTCCCACTAT | |
| | | TCAGCATGTGGGCCTCAGCCTGTGATCTAAGAAATCCCTGGCTAA | |
| | | GAGAGGGAGCCCTAAGCCCCACAGTAGGCTTTTTTGCCTTAAAGC | |
| | | CTGACTTCTACCCTAATTTAAGCATTTTACCAAATGAAGTCAGTCAA | |
| | | CAATTCGACTTCTTTTTAAACTCTGCTCACCCACCAAGCATACAATC | |
| | | AGAAAAAGATGTTAGATGGGAATATACATACACAAACTTAATGAGG | |
| | | CCTATATACAACCAGACCCCATCACTAAAGGCCTCCACATATGACT | |
| | | GGCAAAACTATAGCAATCCAAACAACTATCAAGCATGCCACCAACA | |
| | | ATTCATAGCATTTAAAGCACAAAGATTTGCCAAAATTAAAGCAGAAT | |
| | | ATCAAACAGTATATCCTACACTAACAACACAGACACCCCAATCAGA | |
| | | AGCACTAACACAAGAATTTGGACTATACTCTCCATACTATTTAACAC | |
| | | CAACAAGAATCAGCCTAGACTGGCACACAGTATTCCACCACATCA | |
| | | GATACAACCCGATGGCAGACAAAGGCCTAGGAAACATGATTTGGG | |
| | | TCGACTGGTGTTCCAGAAAAGAAGCCACCTACGACCCCACAAGAT | |
| | | CCAAGTGCATGCTAAAAGACCTACCACTATACATGCGCTTCTATGG | |
| | | CTACTGTGACTGGGTAACTAAATCAATAGGCTCAGAAACAGCCTG | |
| | | GAGAGACATGAGATTAATGGTGGTCTGCCCTTATACAGAACCCCA | |
| | | ACTAATGAAAAAAAATGACAAAACCTGGGGCTATGTAATCTATGGC | |
| | | TACAACTTTGCAAACGGAAACATGCCGTGGTTACAGCCATATATCC | |
| | | CAATCTCGTGGTTTTGCCGTTGGTTCCCTTGCATCACTCACCAACG | |
| | | TGAAGCAATGGAGTCAGTTGTGGCCACAGGACCGTTCATGGTCAG | |
| | | AGACCAAGACCGCAACAGTTGGGACATAACTATAGGCTACAAATTC | |
| | | TTATGGAGATGGGGGGGCTCTCCTCTGCCCACTCAGGCAATCGAC | |
| | | GACCCCTGCCAGCAGGGAACCCACCCGCTTCCCGAGCCCGGTAC | |
| | | GTTGCCTAGAATCTTACAAGTCAGCGACCCGACGCAACTCGGACC | |
| | | GAAAACCATATTCCACCTCTGGGACCAGAGGCGTGGACTTTTTAG | |
| | | CAAAAGAAGTATTGAAAGAATGTCAGAATACAAAGGAACTGATGAC | |
| | | TTATTTTCACCAGGTCGCCCAAAGCGCCCAAAGCTCGACACACGT | |
| | | CCCGAAGGACTACCAGAGGAGCAAAGAGGAGCTTACAATTTACTC | |
| | | CAAGCCCTCGAAGACTCAGCCCAGTCGGAAGAAAGCGACCAAGA | |
| | | AGAAATGCCTCCCCTCGAAGAAGAACAAGTACTCCACGAGCAAAA | |
| | | GAAAGAGGCGCTCCTCCAGCAGCTCCAGCAGCAGAAACACCACC | |
| | | AGCGAGTCCTCAAGCGAGGCCTCAGACTCCTCCTCGGAGACGTC | |
| | | CTGAAACTCCGCCGGGGTCTACACATAGACCCGGTCCTTACATAG | |
| BAB79318.1 | AB064597.1 | ACGGCGTGGTGGTGGGGACGGTGGCGCCGCCGCTGGCGCCGCA | 189 |
| | | GGCGACCGTGGAGACCGAGACTACGACGAAGAAGAGCTAGACGA | |
| | | GCTTTTCCGCGCCGCCGCCGAAGACGATTTGTAAGTAGGAGATGG | |
| | | CGCCGGCCTTACAGGCGCAGGAGGAGACGCGGGCGACGCAGAC | |
| | | GCAGACGCAGACGCAGACATAAGCCCACCCTAGTACTCAGACAGT | |
| | | GGCAACCTGACGTTATCAGACACTGTAAGATAACAGGACGGATGC | |
| | | CCCTCATTATCTGTGGAAAGGGGTCCACCCAGTTCAACTACATCAC | |
| | | CCACGCGGACGACATCACCCCCAGGGGAGCCTCCTACGGGGGCA | |
| | | ACTTCACAAACATGACTTTCTCCCTGGAGGCAATATACGAACAGTT | |
| | | TCTGTACCACAGAAACAGGTGGTCAGCCTCCAACCACGACCTCGA | |
| | | ACTCTGCAGATACAAGGGTACCACCCTAAAACTGTACAGGCACCC | |
| | | AGATGTAGACTACATAGTCACCTACAGCAGAACGGGACCCTTTGA | |
| | | GATCAGCCACATGACCTACCTCAGCACTCACCCCCTTCTCATGCT | |
| | | GCTAAACAAACACCACATAGTGGTGCCCAGCCTAAAGACTAAGCC | |
| | | CAGGGGCAGAAAGGCCATAAAAGTCAGAATAAGACCCCCCAAACT | |
| | | CATGAACAACAAGTGGTACTTCACCAGAGACTTCTGTAACATAGGC | |
| | | CTCTTCCAGCTCTGGGCCACAGGCTTAGAACTCAGAAACCCCTGG | |
| | | CTCAGAATGAGCACCCTGAGCCCCTGCATAGGCTTCAATGTCCTT | |
| | | AAAAACAGCATTTACACAAACCTCAGCAACCTACCTCAGCACAGAG | |
| | | AAGACAGACTTAACATTATTAACAACACATTACACCCACATGACATA | |
| | | ACAGGACCAAACAATAAAAAATGGCAGTACACATATACCAAACTCA | |
| | | TGGCCCCCATTTACTATTCAGCAAACAGGGCCAGCACCTATGACTT | |
| | | ACTACGAGAGTATGGCCTCTACAGTCCATACTACCTAAACCCCACA | |
| | | AGGATAAACCTTGACTGGATGACCCCCTACACACACGTCAGGTAC | |
| | | AATCCACTAGTAGACAAGGGCTTCGGAAACAGAATATACATACAGT | |
| | | GGTGCTCAGAGGCAGATGTAAGCTACAACAGGACTAAATCCAAGT | |
| | | GTCTCTTACAAGACATGCCCCTGTTTTTCATGTGCTATGGCTACAT | |
| | | AGACTGGGCAATTAAAAACACAGGGGTCTCCTCACTAGCGAGAGA | |
| | | CGCCAGAATCTGCATCAGGTGTCCCTACACAGAGCCACAGCTGGT | |
| | | GGGCTCCACAGAAGACATAGGGTTCGTACCCATCACAGAGACCTT | |
| | | CATGAGGGGCGACATGCCGGTACTTGCACCATACATACCGTTGAG | |
| | | CTGGTTTTGCAAGTGGTATCCCAACATAGCTCACCAGAAGGAAGTA | |
| | | CTTGAGGCAATCATTTCCTGCAGCCCCTTCATGCCCCGTGACCAG | |
| | | GGCATGAACGGTTGGGATATTACAATAGGTTACAAAATGGACTTCT | |
| | | TATGGGGCGGTTCCCCTCTCCCCTCACAGCCAATCGACGACCCCT | |
| | | GCCAGCAGGGAACCCACCCGATTCCCGACCCCGATAAGCACCCT | |
| | | CGCCTCCTACAAGTGTCGAACCCGAAACTGCTCGGACCGAGGACA | |
| | | GTGTTCCACAAGTGGGACATCAGACGTGGGCAGTTTAGCAAAAGA | |
| | | AGTATTAAAAGAGTGTCAGAATACTCATCGGATGATGAATCTCTTG | |
| | | CGCCAGGTCTCCCATCAAAGCGAAACAAGCTCGACTCGGCCTTCA | |
| | | GAGGAGAAAACCCAGAGCAAAAAGAATGCTATTCTCTCCTCAAAG | |
| | | CACTCGAGGAAGAAGAGACCCCAGAAGAAGAAGAACCAGCACCC | |
| | | CAAGAAAAAGCCCAGAAAGAGGAGCTACTCCACCAGCTCCAGCTC | |
| | | CAGAGACGCCACCAGCGAGTCCTCAGACGAGGGCTCAAGCTCGT | |
| | | CTTTACAGACATCCTCCGACTCCGCCAGGGAGTCCACTGGAACCC | |
| | | CGAGCTCACATAG | |
| BAB79326.1 | AB064599.1 | ACGGCGTGGTGGAGATACAGACGGAGACCGTGGAGAAGATGGAG | 190 |
| | | GAGACGCCGCTGGGGCCTACGAACCCGAAGACCTAGAAGAACTTT | |
| | | TCGCCGCCGCCGAGCAAGACGATATGTGAGTAGAGGGCGGCGCC | |
| | | GCCGATACAGGCGCAGACGCAGACGGGGGCGACGCAGACGGGG | |
| | | ACGCAGACGCAGGCACAGAAAGACTCTCATTGTCAGGCAATGGCA | |
| | | ACCAGACGTTATAAAGAGATGCTTTATCACAGGGTGGCTGCCCCT | |
| | | CATTATCTGTGGAAACGGACACACCCAATTTAACTTTATAACTCACA | |
| | | TGGATGACATTCCACCCAAGAATGCATCCTACGGGGGCAACTTCA | |
| | | CCAACTTGACCTTTAACCTAGCCTGCTTCTATGACGAATTCATGCA | |
| | | CCACAGAAACAGATGGTCAGCCTCTAACCATGACCTAGAGCTAGT | |
| | | GAGATACATCAGAACCAGCCTTAAACTCTACAGACACGAGTCAGTA | |
| | | GACTATATAGTGTGCTACACCACCACAGGCCCCTTCGAGACAAAT | |
| | | GAAATGTCCTACATGCTCACTCACCCTCTGGCCATGCTCCTCAGCA | |
| | | AAAGACACGTAGTTGTGCCTAGCCTAAAAACAAAACCACACGGCA | |
| | | GAAAGTACAAAAAGATAACAATTAAGCCCCCAAAACTGATGCTAAA | |
| | | CAAGTGGTACTTTGCTACAGACCTCTGCCACATAGGCCTCTTCCAG | |
| | | CTCTGGGCCACAGGCCTAGAGCTTAGAAATCCATGGCTCAGATCA | |
| | | GGCACAAACAGCCCTGTTATAGGCTTCTATGTCCTTAAAAACCAAG | |
| | | TTTACAAAAACAGATACAGCAACCTAAACACAACAGAAGCACACAA | |
| | | CGCCAGACAAGACGCATGGAACGAACTAACCCAAACAAAAACTAA | |
| | | CGACAAATGGTACAATTGGCAATATACATACAATAAACTTATGAAG | |
| | | CCAATTTACTATGCAGCTTCAAATGAAAGTAGTAATTCAGCCATGA | |
| | | AAGGAAAAACATATAATTGGAAACATTACAAAGAATATTTTAGCAAC | |
| | | ACACAAACTAAGTGGAAAACAATTATTAAAGACGCCTATGACTTAG | |
| | | TAAGAGAGGAATACCAACAATTATACACCACAACTATGGCATATCC | |
| | | ACCACCATGGCAATCAACCACTTCTAATACAGGCAGACAATACCTA | |
| | | GAACATGACTGTGGCATTTACAGCCCATACTTTCTAACACCACAAA | |
| | | TATATAGCCCAGAATGGCACACAGCCTGGTCCTACATCAGATACAA | |
| | | TCCCCTCACAGACAAAGGCATAGGAAACAGAGTCTGTGTCCAGTA | |
| | | CTGCAGCGAGGCCAGCAGCGACTACAACCCAATAAAGAGCAAGTG | |
| | | TATGTTACAAGACATGCCCTTGTGGATGATGCTGTATGGCTACGCA | |
| | | GACTATGTAGTAAAGAGCACAGGCATACAGTCAGCCTGGACAGAC | |
| | | ATGAGAGTGGCCATCAGATGTCCCTACACAGACCCTAAGCTTGTG | |
| | | GGCAGCACAGAAAACACCATGTTTATCCCCATAGGCCTAGAATTCA | |
| | | TGAACGGAGACATTCCAGACAAAAGGCCCTACATTCCGTTAACCT | |
| | | GGTGGTTTAAGTGGTACCCCATGATTACACACCAGAAAACCGCAAT | |
| | | TGAGGCAATAGTTTCCTGCAGCCCCTTCATGCCCAGAGATCAGGA | |
| | | ACAAGCTAGTTGGGACATAACTGTAGGTTACAAAGCAACCTTCTTA | |
| | | TGGGGCGGGTCCCCGTTACCTCCACAGCCCATTGACGACCCCTG | |
| | | CCAAAAAGGAAAACACGACATTCCCGACCCCGATACAAACCCTCC | |
| | | AAGAATACAAATATCAGACCCGCAACACCTCGGACCGGCGACGCT | |
| | | GTTCCACTCGTGGGACCTCAGACGTGGATATATTAATACAAAAAGT | |
| | | ATTAAAAGAATCTCAGAACACCTCGATGCTAATGAATATTTTTCGAC | |
| | | AGGCGTCGTGTCCAAAAAACCCCGATTCGACACTCCCCACCACGG | |
| | | GCAGCTATCAAACCAAGAAGAAGACGCCTTGTCTATCCTCAGACAA | |
| | | CCCCAAAAAGAGCAAGAAGAGACCACCTCCGAGGAAGAACAAGCA | |
| | | CTCCAAAAAGAAGAGGAGCAAAAAGAAAAGCTCCTACAGCAACTC | |
| | | AGAGTCCAGCGACAGCACCAGCGAGTCCTCAGACAGGGAATCAAA | |
| | | CACCTCATGGGAGACGTCCTCCGACTCAGACAGGGAGTCCACTG | |
| | | GAACCCAGTCCTATAA | |
| BAB79330.1 | AB064600.1 | ACGGCCTGGGGATGGTACCGGAGAAGAAGATGGCGCCCATGGAG | 191 |
| | | AAGGAGAAGGTGGGCGATACGCAGAAGAAGACCTAGAAGAACTG | |
| | | TTCGCCGCCGCGGCAGAAGACGATATGTGAGTAGATGGCCGCGC | |
| | | CGCCGATACAGGCGCAGACGCAGACGAACCAGACGTAGGGGGG | |
| | | GACGCAAAAGGAGACACAGACAGACTCTTATACTCAGACAGTGGC | |
| | | AACCAGATGTTATGAAAAAATGTTTTATTACTGGCTGGATGCCCCT | |
| | | CATTATATGTGGCACTGGGAACACTCAATTTAACTTTATAACCCATG | |
| | | AAGACGATGTGCCACCAAAAGGAGCCTCCTATGGAGGCAACCTCA | |
| | | CTAACCTCACCTTCACTCTAGAAGGACTGTATGACGAACACCTACT | |
| | | CCACAGAAACAGGTGGTCCAGATCAAACTTTGATCTAGACCTCAG | |
| | | CAGATACCTCTACACTATAATAAAGCTATACAGACACGAGTCTGTA | |
| | | GACTACATAGTCACCTACAACAGAACAGGCCCCTTTGAAATAAGCC | |
| | | CACTCAGCTACATGAACACACACCCTATGCTAATGCTCCTAAACAA | |
| | | GCACCACGTAGTGGTGCCAAGCCCAAAAACAAAGCCCAAAGGCAA | |
| | | GAGGGCCATTAAAATTAAAATAAAGCCACCTAAACTAATGCTAAAC | |
| | | AAATGGTACTTTGCAAGAGACACGTGTAGAATAGGCCTCTTTCAGC | |
| | | TCTATGCCACAGGGGCTAACCTAACAAACCCCTGGCTCAGGTCAG | |
| | | GCACAAACAGCCCTGTAGTGGGATTCTATGTAATTAAAAACTCCAT | |
| | | ATATCAAGACGCCTTTGATAACCTGGCAGACACAGAACATACAAAC | |
| | | CAAAGAAAAAATGTATTTGAAAACAAACTATATCCCACTACAACAAC | |
| | | TAACAAAGACAACTGGCAATACACATACACATCCCTCATGAAAAAC | |
| | | ATATACTTTAAAACAAAACAAGAAGCAGAAAACCAAACAATGAGTA | |
| | | GCACATACAACTTTGACACATACAAAACAAACTATGACAAAGTAAG | |
| | | AACTAAATGGATAAAAATAGCTGAAGATGGCTATAAACTAGTATCA | |
| | | AAAGAATACAAAGAAATATACATCAGTACAGCCACATACCCTCCAC | |
| | | AATGGAATTCAAGAAACTACCTTAGCCATGACTATGGCATTTATAG | |
| | | TCCTTACTTTTTAACACCCCAAAGATACAGCCCCCAATGGCACACA | |
| | | GCATGGACATATGTCAGATACAACCCACTAACAGACAAAGGCATA | |
| | | GGCAACAGAATATTTGTTCAGTGGTGCTCAGAAAAAAACAGCTCAT | |
| | | ACAACAGCACAAAAAGCAAGTGCATGCTACAAGACATGCCCCTTTT | |
| | | TATGCTAACCTATGGGTACCTAGACTATGTACTAAAATGCGCAGGC | |
| | | TCTAAATCAGCCTGGACAGACATGAGAGTCTGTATCAGAAGCCCAT | |
| | | ACACAGAACCACAGCTTACAGGCAACACAGATGATATTAGTTTTGT | |
| | | TATAATATCAGAGGCCTTCATGAACGGGGACATGCCCTACCTAGCT | |
| | | CCACACATACCCGTTAGTCTGTGGTTTAAGTGGTACCCCATGATAT | |
| | | TACACCAGAAGGCAGCTTTAGAAACCATAGTTTCCTGTGGACCGTT | |
| | | TATGCCCAGAGACCAGGAAGCCAACTCTTGGGACATAACCGCAGG | |
| | | TTACAAAGCAGTTTTTAAGTGGGGTGGGTCCCCTCTGCCTCCACA | |
| | | GCCTATCGACGACCCCTACCAAAAACCCACCCACGAAATACCCGA | |
| | | CCCCGATAAGCACCCTCCAAGACTACAAATTGCAGACCCGAAAAT | |
| | | CCTCGGACCGTCGACAGTCTTCCACACATGGGACATCAGACGTGG | |
| | | CCTCTTTAGCACAGCAAGTCTTAAGAGAGTGTCAGAATACCAACCG | |
| | | CCTGATGACCTTTTTTCAACAGGCGTCGCATCCAAAAGACCCCGAT | |
| | | TCGACACTCCAGTCCAAGGGCAGCTCGAAAGCCAAGAAGAAGAAA | |
| | | GCTATCGTTTACTCAGAGCACTCCAAAAAGAGCAAGAGACAAGCA | |
| | | GCTCGGAAGAGGAGCAGCCACAAAACCAAGAGATCCAAGAAAAAC | |
| | | TACTCCTCCAGCTCCAGCAGCAGCGACAACAGCAGCGACTCCTCG | |
| | | CAAAGGGAATCAAGCACCTCCTCGGAGATGTCCTCCGACTCCGAA | |
| | | AAGGAGTCCACTGGGACCCGGTCCTTACATAG | |
| BAB79334.1 | AB064601.1 | ACGGCGTGGTACAGAAGAAGAAGGTGGAGACCGTGGAGAAGACG | 192 |
| | | CCGCAGACCGTGGACCCTACGCAGAAGAAGAGCTAGAAGATTTGT | |
| | | TCGCCGCCGCCCGAGAAGACGATATGTGAGTAGATGGCGGCGCC | |
| | | GCCGATACAGGCGCAGACTAAGACGGGGGAGACGACGAAGGGG | |
| | | ACGCAGACGCAGAAAAGAAACTATAATAGTGAGACAGTGGCAGCC | |
| | | AGATGTAATGAGAAACTGTTATATTACTGGCTTCCTACCTCTCATAG | |
| | | TCTGTGGCTCAGGCAACACTCAATTTAACTTTATCACACATGAGAA | |
| | | TGACATACCCCCAAGGGGAGCCTCCTATGGGGGCAACCTCACCAA | |
| | | CATAACCTTCACCCTAGCGGCACTATATGACCAGTACTTGCTACAC | |
| | | AGAAACAGGTGGTCCAGGTCAAACTTTGACCTAGACCTAGCCAGA | |
| | | TACATTAACACAAAACTAAAACTATACAGACATGACTCAGTAGACTA | |
| | | CATAGTAACCTACAACAGAACAGGTCCCTTTGAGGTGAATCCACTA | |
| | | ACATACATGCACACTCACCCCCTACTCATGCTCGTGAACAGGCAC | |
| | | CACATAGTGGTGCCCAGTTTAAAAACAAAACCCAGAGGCAAAAGA | |
| | | TACATAAAAGTAAAAATAAAGCCTCCAAAACTAATGCTAAACAAGT | |
| | | GGTACTTTGCGAAAGACATCTGCCCACTAGGCCTCTTCCAGCTATA | |
| | | TGCTACCGGCCTAGAACTCAGAAACCCCTGGATCAGAGAGGGCAC | |
| | | AAACAGCCCCATAGTAGGGTTTTATGTTTTAAAACCCTCACTATATA | |
| | | ATGGAGCCATGTCAAACTTAGCAGACACAGAACATTTAAACCAAAG | |
| | | ACAAACCCTATTTAACAAACTACTTCCAACACAAAACCAAAAAGAC | |
| | | GAATGGCAATACACATACAACAAACCAATGCAAAAAATATATTATG | |
| | | AAGCAGCAAACAAGCAAGATAGTGGCTTTAAAAATACAACATATAA | |
| | | CTGGACAAACTACAAAACTAACTACCAAAAAGTACAATCACAATGG | |
| | | CAAACTGTAGCACAACAAAACTACAACCAAGTATACAATGAATTTA | |
| | | AAGAGGTATACCCACTAACAGCTACATGGCCACCGCAATGGAATG | |
| | | CTAGACAATACATGTCACACGACTTTGGCATATACAGCCCATACTT | |
| | | TTTGTCACCTGCAAGATTTACAGACTACTGGCACAGTGCATACACC | |
| | | TATGTCAGATACAACCCCATGTCAGACAAAGGCATAGGTAACATAA | |
| | | TCTGCATACAATGGTGCAGTGAAAAAAACAGTGAATTTAATGAGAC | |
| | | TAAAAACAAGTGCATACTAAGAGACATGCCACTTTACATGCTAACA | |
| | | TATGGCTACCTAGACTATACCACAAAATGCACAGGCTCCAACTCCA | |
| | | TCTGGACAGACGCCAGAGTAGCCATCAGATGTCCATACACAGATC | |
| | | CCCCACTATCAAATCCAACTAACAAAAACACACTTTATATTCCACTA | |
| | | TCTACATCTTTCATGCAAGGAGACATGCCCTGGCCAACCACAAACA | |
| | | TTCCGTTAAAGATGTGGTTTAAGTGGTATCCCATGATCATGCACCA | |
| | | GAGGGCCTGTTTAGAAACCATAGTTTCCTGTGGACCGTTTATGCCC | |
| | | AGAGACCAAACCGCAAGCAGTTGGGACATAACTATTGCATACAGA | |
| | | GCCTTTTTTAAATGGGGTGGCAATCCTCTGCCTCCACAGCCCATC | |
| | | GACGACCCCTGCCAAAAAGACACCCACGAAATACCCGACCCCGAT | |
| | | AAACACCCTAGAGGAATACAAATATCAGACCCGAAGGTACTCGGA | |
| | | CCACCCACAGTCTTCCACACATGGGACATCAGACGTGGACTGTTT | |
| | | AGCTCGACGAGTCTTAAAAGAGTGTCAGAATACCAACCGCCTGAT | |
| | | GACCCTTTTTCAACAGGCGTCGTCTTCAAAAGACCCCGACTGGAA | |
| | | ACCCAGTACAAAGGAACCCAAGAAACCCCAGAAGAAGACGCCTAC | |
| | | ACTTTACTCAAAGCACTCCAAAAAGAGCAAGAGAGCAGCAGCTCG | |
| | | GAAGAAGAACTCCCACAAGAAGAGCAAGAGATCCAAAAAACACAA | |
| | | CTCCTCAAGCAGCTCCAACTCCAGCAGCAGCAACAGCGAATCCTC | |
| | | AAGAGGGGAATCAGACACCTCTTCGGAGACGTCCTCCGACTCAGA | |
| | | AAAGGAGTCCACTCCAACCCAGACCTATTATAA | |
| BAB79338.1 | AB064602.1 | ACGGCCTGGTACCGGTACAGAAGAAGGCCATGGCGCCGAAGGAG | 193 |
| | | GCGACCGAGGTGGGGCCTACGCAGAAGAAGATTTAGAAGATCTTT | |
| | | TCGCGGCCGCGGAAGAAGACGATATGTGAGTAGATGGTCGCGCC | |
| | | GCCGATACAGGCGCAGACGGAGAAGGGGGCGACGTAGACGGGG | |
| | | ACGCAGACGAAGAAAGAGACAGACTCTTATACCGAGACAGTGGCA | |
| | | GCCAGATGTTACTAAAAAGTGCTTCATTACTGGCTGGATGCCCTTA | |
| | | ATAATCTGTGGGACTGGACACACACAATTTAACTTTATAACCCACG | |
| | | AAGAGGATATCCCCGGTGCAGGAGCCTCCTATGGAGGAAACCTTA | |
| | | CAAACATTACCATTACTCTGGGAGGGCTATATGAACAATATATGCT | |
| | | TCACAGAAACCACTGGTCCAGAAGCAACTATGACCTAGAGCTGGC | |
| | | CAGATACCTAGGCTTCACCCTAAAATGCTACAGACATGCAACAGTA | |
| | | GACTATATACTTACATACAGCAGAACAACACCCTTTGAGACCAATG | |
| | | AACTGAGCCACATGCTAACTCACCCCTTACTAATGCTACTAAACAA | |
| | | ACATCACAGAGTAATACCCAGCTTAAAAACAAGGCCAAAAGGAAAA | |
| | | AGGTCAGTTAGAATCCACATTAAACCCCCAAAACTAATGATAAACA | |
| | | AATGGTACTTTGCAAAAGACCTCTGTAACATAGGACCCTGTCAAAT | |
| | | ATATGCCACAGGCCTAGAACTCTCAAACCCCTGGCTAAGATCAGG | |
| | | CACAAACAGCCCTGTAATAGGCTTTTGGGTACTTAAAAATCACCTA | |
| | | TATGATGGCAACCTCTCAAACATAGCCTCAGGTGAACAATTAACAG | |
| | | CCAGACAAACTCTATTTACAACTAAATTACTCCCAAGTAATAACACC | |
| | | AAAGACGAATGGCAATACGCCTATACCCCACTAATGAAAACATTCT | |
| | | ACACACAAGCAGCCAACACAGCAGCACATAACATAACAGACAAAA | |
| | | CATACAACTGGAAAAACTACAAAACTCACTATGACAAAGTACAACA | |
| | | AACATGGACAACAAAAGCACAATTTAATTATGACTTAGTTAAAGAA | |
| | | GAATACAAAACGGTATATCCAACCACAGCTACATTCCCACCAGAGT | |
| | | GGTCAAACAGACAATATCTAGAACATGACTATGGCTTATTCAGCCC | |
| | | TTATTTTCTAACACCAAACAGATACAGCACAGAGTGGCACATGCCA | |
| | | ATTACCTATGTTAGATACAACCCACTAGCAGACAAAGGCATAGGCA | |
| | | ACAGAATATACATGCAGTGGTGCTCAGAAAGCAGCAGCAGCTTTG | |
| | | AGCCCACCAAAAGCAAGTGCATGCTACAAGACATGCCACTATACAT | |
| | | GCTCACATATGGATACCTAGACTATGTTGTTAAATGCACAGGTGTT | |
| | | AAATCAGCCTGGACAGACATGAGAGTGGCCATTAGAAGCCCCTAC | |
| | | ACCTTTCCTCAACTAATAGGCAGCACAGATAAAGTGGGCTTCATCC | |
| | | CCCTAGGTGAAAAATTCATGAGCGGAGACACAGACCCCGTTAAAA | |
| | | ACTTTATACCGTTAAAGTATTGGTACAGATGGTATCCGTTTGCGGC | |
| | | TAACCAAAAGTCAGTTTTAGAAACCATAGTTTCCTGTGGCCCCTTC | |
| | | ATGCCCAGAGATCAGGAAGCAGGCTCTTGGGACATAACTGTAGGT | |
| | | TACAAAGCAACCTTTAAACGGGGGGGCTCCCCTCTACCTCCACAG | |
| | | CCCATCGACGACCCATGCCAAAAGCCCACCCACGACCTTCCCGAC | |
| | | CCCGATAGACACCCCCCAAGAATACAAATCTCGGACCCGGCAAGA | |
| | | CTCGGACCGGAGACGCTCTTCCACTCATGGGACATCAGACGTGGA | |
| | | TACATTAACACAAAAGCTATTAAAAGAATCTCAGATTACACAGAATC | |
| | | TAATGACTATTTTTCAACAGGCGTCGTGTCAAAAAGACCCCGATTG | |
| | | GAAACCCAGTACCACGGCCAACACGAAAGCCAAGAAGAAGACGC | |
| | | CTATCTTTTACTCAAACAACTCCAGGAAGAGCAAGAAACGAGCAGT | |
| | | TCGGAGGGAGAACAAGCACCCCAAGAAAAAACACTCCAAAAAGAA | |
| | | AAGCTCCTCAAGCAGCTGCAGCTCCACAAGCAGCAGCAGCAACTC | |
| | | CTCAGAAAAGGAATCAGACACCTCCTCGGGGACGTCCTCCGACTC | |
| | | AGACGGGGAGTCCACTGGGACCCAGGCCTATAG | |
| BAB79342.1 | AB064603.1 | ACGGCGTGGTGGTGGGGCCGATGGAGACAGCGCCGCTGGGGCC | 194 |
| | | GCCGCCGCCGCAGACCATGGAGGGTACGACGAAGGAGACCTAGA | |
| | | AGATCTTTTCGCCGCCGCCGCCGAGGACGATATGTGAGTAGGCG | |
| | | GAGGCGCCGCCGCTACTACAGGCGCAGACTAAGACGGGGCAGAC | |
| | | GCAGAGGGCGACGAAAGAGACACAGACCGACCCTAATACTGAGG | |
| | | CAGTGGCAACCTGACGTTGTTAAACACTGTAAGATAACAGGATGG | |
| | | ATGCCCCTCATTATCTGTGGCTCTGGCAGCACACAGATGAACTTTA | |
| | | TAACCCACATGGACGATACTCCTCCCATGGGATACACCTACGGGG | |
| | | GCAACTTTGTAAATGTGACTTTCAGCTTAGAGGCCATCTATGAACA | |
| | | GTTCCTATATCACAGAAACAGATGGTCCAGATCTAACCATGACTTA | |
| | | GACCTAGCCAGGTACCAAGGAACCACCTTAAAACTCTACAGACAC | |
| | | GCCACAGTAGACTACATACTTTCCTACAACAGGACAGGACCCTTCC | |
| | | AGATCAGTGAGATGACATACATGAGCACTCACCCAGCAATAATGCT | |
| | | ACTAATGAAACACAGAATAGTTGTGCCCAGCCTTAGAACAAAGCCT | |
| | | AAAGGCAGGCGCTCCATAAAAATTAGAATAAAGCCCCCCAAACTTA | |
| | | TGCTAAACAAGTGGTACTTTACCAAAGACATATGCTCCATGGGCCT | |
| | | CTTCCAACTAATGGCCACCGGAGCAGAACTCACTAACCCCTGGCT | |
| | | CAGAGACACCACAAAAAGCCCAGTAATAGGCTTCAGAGTTCTAAAA | |
| | | AACAGTGTTTACACCAACTTATCTAACCTAAAAGACGTATCCATATC | |
| | | AGGAGAAAGAAAATCCATCTTAAACAAAATTCACCCAGAAACTCTC | |
| | | ACAGGATCAGGCAATGCATCTAAAGGGTGGGAATACTCATACACA | |
| | | AAACTAATGGCGCCCATATACTATTCAGCAGTTAGAAACAGCACAT | |
| | | ACAACTGGCAAAACTACCAAACACACTGCGCAACAACAGCTATCAA | |
| | | ATTTAAAGAAAAACAAACCAGTACTCTAACTCTTATTAAAGCAGAGT | |
| | | ACTTATACCACTACCCAAACAATGTCACACAGGTAGACTTCATAGA | |
| | | TGACCCCACACTCACACATGACTTTGGCATATACAGCCCATACTGG | |
| | | ATAACACCTACCAGAATAAGCCTAGACTGGGACACACCATGGACA | |
| | | TATGTCAGATACAACCCACTCTCAGACAAAGGCATAGGCAACAGAA | |
| | | TCTATGCACAGTGGTGCTCAGAAAAAAGCAGCAAATTAGACACCAC | |
| | | AAAGAGCAAATGCATACTAAAAGACTTTCCACTATGGTGCATGGCC | |
| | | TATGGCTACTGTGACTGGGTAGTAAAATGTACAGGAGTGTCCAGT | |
| | | GCATGGACAGACATGAGAGTAGCCATCATCTGCCCGTACACAGAA | |
| | | CCGGCACTTATAGGGTCAGATGAAAATGTAGGCTTTATTCCAGTAA | |
| | | GTGACACCTTTTGCAACGGAGACATGCCGTTTCTTGCACCATACAT | |
| | | CCCTATTACATGGTGGATCAAGTGGTACCCCATGATTACACACCAA | |
| | | AAGGAAGTTCTTGAGGCAATAGTAAACTGTGGACCGTTTGTCCCC | |
| | | CGAGACCAAAGTTCCCCAGCTTGGGAAATCACCATGGGTTACAAA | |
| | | ATGGATTGGAAATGGGGCGGCTCTCCCCTGCCTTCACAGGCAATC | |
| | | GACGACCCCTGCCAGAAGCCCACCCATGAGCTACCCGATCCCGAT | |
| | | AGACACCCTCGCATGTTACAAGTCTCTGACCCGACAAAGCTCGGA | |
| | | CCGAAGACAGTGTTCCACAAATGGGACTGGAGACGTGGGCAACTT | |
| | | AGCAAAAGAAGTATTAAAAGAGTCCAAGAAGACTCAACGGATGAT | |
| | | GAATATGTTACAGGGCCTTTATCAAGAAAAAGAAACAAGCTCGACA | |
| | | CAAAGATGCCAGGCCCCCCAACCCCCGAAAAAGAAAGCTACACTT | |
| | | TACTCCAAGCCCTCCAAGAGTCGGGCCAGGAGAGCAGCTCCCAG | |
| | | GACGAAGAACAAGCACCCCAAAAAGAAGAGAACCAGAAAGAAGCG | |
| | | CTCGTGGAGCAGCTCCAGCTCCAGAAACAGCACCAGCGAGTCCTC | |
| | | AAGCGAGGCCTCAAACTCCTCTTGGGAGACGTCCTCCGACTCCGC | |
| | | CGCGGAGTCCACTGGGACCCCCTCCTATCCTAA | |
| BAB79346.1 | AB064604.1 | ATGGCATGGGGATGGTGGAAACGAAAGCGGCGCTGGTGGTGGAG | 195 |
| | | AAAGCGGTGGACCCGTGGCCGACTTCGCAGACGATGGCCTAGAC | |
| | | GATCTCGTCGCCGCCCTCGACGAAGAAGAGTAAGGAGGCGGAGG | |
| | | AGGTGGAGGAGAGGGCGACCGAGACGCAGACTGTACAGACGCG | |
| | | GGAGACGGTACAGACGAAAACGGAAGAGGGCTAAGATAACTATAA | |
| | | GACAATGGCAGCCAGCCATGACGAGACGCTGTTTTATAAGGGGAC | |
| | | ACATGCCCGCTTTAATATGTGGCTGGGGGGCGTACGCCAGCAACT | |
| | | ACACCAGCCACCTGGAGGACAAAATAGTTAAAGGACCCTACGGAG | |
| | | GGGGACACGCCACTTTTAGATTCTCCCTACAAGTACTGTGCGAGG | |
| | | AGCATCTAAAACACCACAATTACTGGACTAGAAGTAACCAAGACCT | |
| | | AGAACTAGCTCTGTACTACGGAGCCACTATTAAATTTTACAGAAGC | |
| | | CCAGACACAGACTTTATAGTAACATACCAGAGAAAATCCCCCCTTG | |
| | | GAGGCAACATACTAACAGCTCCTTCACTACACCCAGCAGAGGCCA | |
| | | TGCTAAGCAAAAACAAAATACTAATACCGAGCTTACAAACAAAACC | |
| | | CAAAGGAAAAAAGACTGTAAAAGTTAACATACCACCCCCCACCCTT | |
| | | TTTGTACATAAGTGGTACTTTCAGAAGGACATATGTGACCTAACAC | |
| | | TGTTTAACTTGAACGTTGTTGCGGCTGACTTGCGGTTTCCGTTCTG | |
| | | CTCACCACAAACTGACAACGTTTGCATCACCTTCCAGGTACTAGCC | |
| | | GCAGAGTACAACAACTTCCTCTCTACAACTTTAGGCACTACAAATG | |
| | | AATCCACTTTTATAGAAAACTTTTTAAAAGTTGCATTTCCAGATGAC | |
| | | AAACCTAGGCATTCAAACATTTTAAACACATTTAGAACAGAAGGAT | |
| | | GCATGTCTCACCCCCAACTACAAAAATTTAAACCACCAAACACAGG | |
| | | ACCAGGCGAAAACAAATACTTTTTTACACCAGACGGACTATGGGGA | |
| | | GACCCCATATACATATACAATAACGGAGTACAACAACAAACTGCAC | |
| | | AACAAATTAGAGAAAAAATTAAAAAAAACATGGAAAATTACTATGCC | |
| | | AAAATAGTAGAAGAAAACACAATAATAACAAAAGGATCAAAAGCAC | |
| | | ACTGCCATCTAACAGGCATATTTTCACCACCATTCTTAAACATAGGT | |
| | | AGAGTAGCCAGAGAATTTCCAGGACTATACACAGACGTTGTCTATA | |
| | | ATCCATGGACAGATAAAGGCAAAGGAAACAAAATATGGTTAGACA | |
| | | GCCTAACAAAAAGCGACAATATATATGACCCAAGACAAAGCATTCT | |
| | | ACTAATGGCAGACATGCCACTATACATAATGTTAAATGGATATATA | |
| | | GACTGGGCAAAAAAAGAAAGAAACAACTGGGGCTTAGCTACACAA | |
| | | TACAGACTACTACTAACATGTCCCTACACATTCCCAAGACTATACG | |
| | | TAGAAACAAACCCAAACTATGGATATGTACCATATTCAGAATCATTT | |
| | | GGAGCAGGCCAAATGCCAGACAAAAACCCCTACGTACCAATTACA | |
| | | TGGAGAGGCAAATGGTACCCTCACATACTTCATCAAGAGGCAGTT | |
| | | ATAAATGACATAGTAATATCAGGCCCATTCACACCAAAAGACACAA | |
| | | AACCAGTAATGCAATTAAACATGAAATACTCGTTTAGATTCACATGG | |
| | | GGCGGCAATCCTATTTCCACACAGATTGTTAAAGACCCCTGCACC | |
| | | CAGCCCACCTTTGAAATACCCGGTGGCGGTAACATCCCTCGCAGA | |
| | | ATACAAGTCATCAATCCGAAAGTCCTCGGACCCAGCTACAGTTTCA | |
| | | GATCCTTTGACCTCAGACGTGACATGTTTAGCGGCTCGAGTCTTAA | |
| | | AAGAGTCTCAGAACAACAAGAGACTTCTGAGTTTTTATTCTCCGGC | |
| | | GGCAAACGCCCCAGGATCGACCTTCCCAAGTACGTCCCGCCAGAA | |
| | | GAAGACTTCAATATCCAAGAGAGACAACAAAGAGAACAGAGACCG | |
| | | TGGACGAGCGAAAGCGAGAGCGAAGCAGAAGCCCAAGAAGAGAC | |
| | | GCAGGCGGGCTCGGTCCGAGAGCAGCTCCAGCAGCAGCTCCAAG | |
| | | AGCAGTTTCAACTCCGAAGAGGGCTCAAGTGCCTCTTCGAGCAGT | |
| | | TAGTCAGAACCCAACAGGGAGTCCACGTAGATCCCTGCCTCGTGT | |
| | | AG | |
| BAB79354.1 | AB064606.1 | ATGGCATGGGGATGGTGGAAGCGACGGCGGCGCTGGTGGTTCCG | 196 |
| | | GAAGCGGTGGACCCGTGGCAGACTTCGCAGACGATGGCCTCGAT | |
| | | CAGCTCGTCGCCGCCCTAGACGACGAAGAGTAAGGAGACGCAGA | |
| | | CGATGGAGGAGGGGGCGACCTAGACGCAGACTGTACCGACGCTA | |
| | | CAGACGCAAAAAACGTAGGAGACGAAAGCCCAAAACAGTTTTAAA | |
| | | ACAATGGCAGCCAGACATTACAAAGAGGTGCTACATAATAGGCTA | |
| | | CATTCCTGCCATAATATGCGGGGCGGGCACCTGGTCTCACAACTA | |
| | | CACCAGCCACCTGCTAGATATTATCCCCAAGGGACCGTTTGGAGG | |
| | | GGGACACAGCACCATGAGATTCTCCCTAAAAGTGCTCTTCGAAGA | |
| | | GCACCTGAGACACCTAAACTTTTGGACACGTAGTAACCAGGATTTA | |
| | | GAACTTGTAAGATACTTTAGATGCTCCTTTAGGTTCTACAGAGACC | |
| | | AACACACAGACTATCTTGTACACTACAGCAGAAAAACACCCCTGGG | |
| | | AGGCAACAGACTGACAGCACCTAGCCTTCACCCAGGGGTACAGAT | |
| | | GCTAAGCAAAAACAAAATAATAGTACCCAGCTATGATACTAAACCT | |
| | | AAGGGCAAAAGCTATGTAAAAGTAACTATAGCACCCCCCACTCTAC | |
| | | TAACTGACAAGTGGTACTTTAGCAAAGACATTTGTGACACAACCTT | |
| | | GGTTAACTTAGACGTTGTACTCTGCAACTTGCGGTTTCCGTTCTGC | |
| | | TCACCACAAACTGACAACCCTTGCATCACGTTTTCCGTTCTTCACT | |
| | | CCATCTACAACGACTTCCTCTCTATAGTAGATACTGGAAACTATAAA | |
| | | ACACAATTTGTGTCAAACTTATCTACAAAAGTAGGTACTGACTGGG | |
| | | GAAAAAGACTAAACACATTTAGAACAGAAGGCTGCTACTCTCACCC | |
| | | TAAATTACCCAAAAAGGCAGTAACACCTGGAAATGACAAAACATAC | |
| | | TTTACTGTACCCGATGGCTTATGGGGAGACGCTGTATTTAATGCAG | |
| | | AGGCAAGCAATATAATTACTAAAAACATGGAGTCATACAGCGAGTC | |
| | | TGCAAAAGCCAGAGGAGTGCAAGGAGACCCTGCATTTTGCCACCT | |
| | | TACAGGCATATACTCACCTCCCTGGCTAACACCAGGTAGAATATCC | |
| | | CCGGAGACTCCAGGACTTTACACAGACGTGACTTACAACCCATAC | |
| | | GCAGACAAAGGAGTGGGTAACAGAATATGGGTTGACTACTGCAGT | |
| | | AAAAAAGGCAATAAATATGACAATACAAGTAAATGCCTTTTAGAAG | |
| | | ACATGCCACTATGGATGGTCACCTTTGGCTATGTAGACTGGGTAAA | |
| | | AAAAGAAACTGGCAACTGGGGTATTCCACTGTGGGCCAGAGTACT | |
| | | GATAAGATGCCCTTACACAGTACCAAAACTTTACAATGAAGCAGAC | |
| | | CCAAACTACGGATGGGTCCCTTACTCCTACTACTTTGGAGAAGGAA | |
| | | AAATGCCAAACGGAGACCTGTACGTACCCTTTAAAATTAGAATGAA | |
| | | GTGGTACCCGTCCATGTGGAACCAAGAACCAGTACTAAATGACTTA | |
| | | GCAAAGAGCGGACCGTTTGCATACAAAGACACAAAAACCAGTGTG | |
| | | ACTGTGACTGCTAAATACAAATTTACATTTAACTTCGGGGGCAACC | |
| | | CCGTACCCTCACAGATTGTACAAGATCCCTGCACACAGTCCACCTA | |
| | | TGACATCCCCGGCACCGGTAACTTGCCTCGCAGAATACAAGTCAT | |
| | | TGACCCGAAAGTCCTCGGTCCCCACTACTCATTCCACCGCTGGGA | |
| | | CTTCAGGCGTGGCCTCTTTGGCCAACAAGCTATTAAGAGAGTGTC | |
| | | AGAACAACCAACAACTTCTGAGTTTTTATTCTCAGGTCCAAAGAGA | |
| | | CCCAGAATCGATCAAGGGCCTTACATCCCGCCAGAAAAAGGCTCA | |
| | | GATTCACTCCAAAGAGAATCGAGACCGTGGAGCAACTCGGAGACC | |
| | | GAGGCAGAGACAGAAGCCCCCTCGGAAGAAGAGCCGGAGAACCA | |
| | | AGAAGAACAAGTACTCCAGTTGCAGCTCCGACAGCAGCTCCGAGA | |
| | | ACAGCGAAAACTCAGACAGGGAATCCAGTGCCTCTTCGAGCAACT | |
| | | GATAACAACCCAACAGGGGGTTCACAAAAACCCATTGCTAGAGTA | |
| | | G | |
| ABD34286.1 | DQ186994.1 | ATGGCGTGGTCGTGGTGGTGGAGGCGACGGAAACGCTGGTGGCC | 197 |
| | | GCGCAGAAGGAGGCGATGGAGGAGATTTCGCACCCGAAGAGCTA | |
| | | GACGAGCTGTTCCGCGCCGTCGCCGCCGACGAAGAGTAAGGAGG | |
| | | CGCCGGTGGGGGAGGCGAAGACGTAGGAGACGGGTTTTTTATAA | |
| | | GAGACGCAGACGAAAGACTGGCAGACTGTACAGAAAGCCCAAAAA | |
| | | GAAACTAGTACTGACTCAGTGGCACCCCACTACCGTCCGCAACTG | |
| | | CTCCATCCGAGGCCTTGTGCCTCTAGTACTCTGCGGACACACTCA | |
| | | GGGCGGCAGAAACTTTGCTCTCAGGAGCGATGACTACCCCAAGCA | |
| | | GGGGTCTCCTTACGGAGGCAGTTTTAGCACTACAACCTGGAACTT | |
| | | GAGGGTCCTTTTTGACGAACACCAAAAACACCACAACACGTGGAG | |
| | | CTACCCCAATAACCAGCTAGACCTGGGCAGATACAAGGGCTGCAC | |
| | | CTTCTACTTTTACAGAGACAAAAAGACAGACTACATAGTAAAGTTTC | |
| | | AGAGGAGGGGACCCTTTAAAATAAACAAGTACAGCAGTCCCATGG | |
| | | CCCATCCGGGCATGATGATGCTAGATAAGATGAAAATCCTGGTGC | |
| | | CCAGCTTTGATACCAGGCCCGGGGGTCGCAGAAGAGTAAAAGTAA | |
| | | CTATCCGCCCCCCCACTCTGTTAGAGGACAAGTGGTACACCCAGC | |
| | | AAGACCTGGCGCCCGTTAATCTTGTGTCACTTGTGGTTTCTGCGG | |
| | | CTAGCTTCATACATCCGTTTAGCCAACCACAAACGAACAACATTTG | |
| | | CACAACCTTCCAGGTGTTGAAAGACATGTACTATGACTGCATAGGA | |
| | | ATTAATTCCACTTTAACAACCAAGTATGAAAACTTATTTAATAAACTA | |
| | | TATTCCAAATGCTGCTACTTTGAAACCTTTCAAACAATAGCCCAGCT | |
| | | AAATCCTGGCTTTAAAGCTGCTAAAAAGACTACTAATGGTTCTGGT | |
| | | TCTACAGCTGCAACACTAGGAGACGCAGTAACTGAACTTAAAAACC | |
| | | CAAATGGTACTTTTTACACAGGCAACAATAGCACCTTTGGCTGCTG | |
| | | CACATATAAACCCACTAAAGAAATAGGTAGTAATGCCAATAAGTGG | |
| | | TTCTGGCATCAGTTAACAGCCACAGATTCAGACACACTAGGCCAAT | |
| | | ACGGCCGTGCCTCCATTAAGTATATGGAGTACCACACAGGCATTTA | |
| | | CAGCTCAATTTTTCTTAGCCCACTAAGAAGCAATCTAGAATTCCCTA | |
| | | CAGCATACCAAGATGTAACATATAATCCACTAACTGACAGAGGTAT | |
| | | AGGTAACAGAATCTGGTACCAGTACAGTACCAAAGAAAACACTACA | |
| | | TTTAATGAAACACAGTGCAAATGTGTACTATCAGACTTGCCACTGT | |
| | | GGAGCATGTTTTATGGCTATGTAGATTTTATAGAGTCAGAACTAGG | |
| | | CATCTCAGCAGAGATACACAACTTTGGCATAGTATGTGTCCAGTGC | |
| | | CCCTACACGTTTCCCCCAATGTTTGACAAATCCAAACCAGATAAAG | |
| | | GCTACGTGTTCTATGACACCCTTTTTGGCAACGGAAAGATGCCAGA | |
| | | CGGGAGCGGACACGTACCCACCTACTGGCAGCAGAGGTGGTGGC | |
| | | CCAGATTCAGCTTCCAGAGACAAGTGATGCACGACATTATCCTCAC | |
| | | CGGGCCCTTCAGCTACAAAGATGACTCTGTAATGACTGGCATAAC | |
| | | CGCAGGCTACAAGTTTAAATTCTCATGGGGCGGTGATATGGTCTC | |
| | | CGAACAGGTCATTAAAAACCCAGAGAGAGGGGACGGACGAGACT | |
| | | CCACCTATCCCGATAGACAGCGCCGCGACTTACAAGTTGTTGACC | |
| | | CACGCTCCATGGGCCCCCAATGGGTATTCCACACCTTTGACTACA | |
| | | GACGGGGGCTTTTTGGAAAGGACGCTATTAAGCGAGTGTCAGAAA | |
| | | AACCGACAGATCCTGACTACTTTACAACACCTTACAAAAAACCAAG | |
| | | ATTTTTCCCTCCAACAGCAGGAGAAGAAAAACTGCAAGAAGAAGA | |
| | | CTCCGCTTTACAGGAGAAAAGAAGCCCGCTCTCGTCAGAAGAGGG | |
| | | GCAGACGAGGGCGCAAGTCCTCCAGCAGCAGGTCCTCCAGTCGG | |
| | | AGCTCCAGCAGCAGCAGGAGCTCGGGGAGCAGCTCAGATTCCTC | |
| | | CTCAGGGAAATGTTCAAAACCCAAGCGGGCATACACATGAACCCC | |
| | | CGCGCATTTCAGGAGCTGTAA | |
| ABD34288.1 | DQ186995.1 | ATGGCGTGGTCGTGGTGGTGGAGGCGACGGAAACGCTGGTGGCC | 198 |
| | | GCGCAGAAGGAGGCGATGGAGGAGATTTCGCACCCGAAGAGCTA | |
| | | GACGAGCTGTTCCGCGCCGTCGCCGCCGACGAAGAGTAAGGAGG | |
| | | CGCCGGTGGGGGAGGCGAAGACGTAGGAGACGGGTTTTTTATAA | |
| | | GAGACGCAGACGAAAGACTGGCAGACTGTACAGAAAGCCCAAAAA | |
| | | GAAACTAGTACTGACTCAGTGGCACCCCACTACCGTCCGCAACTG | |
| | | CTCCATCCGAGGCCTTGTGCCTCTAGTACTCTGCGGACACACTCA | |
| | | GGGCGGCAGAAACTTTGCTCTCAGGAGCGATGACTACCCCAAGCA | |
| | | GGGGTCTCCTTACGGAGGCAGTTTTAGCACTACAACCTGGAACTT | |
| | | GAGGGTCCTTTTTGACGAACACCAAAAACACCACAACACGTGGAG | |
| | | CTACCCCAATAACCAGCTAGACCTGGGCAGATACAAGGGCTGCAC | |
| | | CTTCTACTTTTACAGAGACAAAAAGACAGACTACATAGTAAAGTTTC | |
| | | AGAGGAGGGGACCCTTTAAAATAAACAAGTACAGCAGTCCCATGG | |
| | | CCCATCCGGGCATGATGATGCTAGATAAGATGAAAATCCTGGTGC | |
| | | CCAGCTTTGATACCAGGCCCGGGGGTCGCAGAAGAGTAAAAGTAA | |
| | | CTATCCGCCCCCCCACTCTGTTAGAGGACAAGTGGTACACCCAGC | |
| | | AAGACCTGGCGCCCGTTAATCTTGTGTCACTTGTGGTTTCTGCGG | |
| | | CTAGCTTCATACATCCGTTTAGCCAACCACAAACGAACAACATTTG | |
| | | CACAACCTTCCAGGTGTTGAAAGACATGTACTATGACTGCATAGGA | |
| | | ATTAATTCCACTTTAACAACCAAGTATGAAAACTTATTTAATAAACTA | |
| | | TATTCCAAATGCTGCTACTTTGAAACCTTTCAAACAATAGCCCAGCT | |
| | | AAATCCTGGCTTTAAAGCTGCTAAAAAGACTACTAATGGTTCTGGT | |
| | | TCTACAGCTGCAACACTAGGAGACGCAGTAACTGAACTTAAAAACC | |
| | | CAAATGGTACTTTTTACACAGGCAACAATAGCACCTTTGGCTGCTG | |
| | | CACATATAAACCCACTAAAGAAATAGGTAGTAATGCCAATAAGTGG | |
| | | TTCTGGCATCAGTTAACAGCCACAGATTCAGACACACTAGGCCAAT | |
| | | ACGGCCGTGCCTCCATTAAGTATATGGAGTACCACACAGGCATTTA | |
| | | CAGCTCAATTTTTCTTAGCCCACTAAGAAGCAATCTAGAATTCCCTA | |
| | | CAGCATACCAAGATGTAACATATAATCCACTAACTGACAGAGGTAT | |
| | | AGGTAACAGAATCTGGTACCAGTACAGTACCAAAGAAAACACTACA | |
| | | TTTAATGAAACACAGTGCAAATGTGTACTATCAGACTTGCCACTGT | |
| | | GGAGCATGTTTTATGGCTATGTAGATTTTATAGAGTCAGAACTAGG | |
| | | CATCTCAGCAGAGATACACAACTTTGGCATAGTATGTGTCCAGTGC | |
| | | CCCTACACGTTTCCCCCAATGTTTGACAAATCCAAACCAGATAAAG | |
| | | GCTACGTGTTCTATGACACCCTTTTTGGCAACGGAAAGATGCCAGA | |
| | | CGGGAGCGGACACGTACCCACCTACTGGCAGCAGAGGTGGTGGC | |
| | | CCAGATTCAGCTTCCAGAGACAAGTGATGCACGACATTATCCTCAC | |
| | | CGGGCCCTTCAGCTACAAAGATGACTCTGTAATGACTGGCATAAC | |
| | | CGCAGGCTACAAGTTTAAATTCTCATGGGGCGGTGATATGGTCTC | |
| | | CGAACAGGTCATTAAAAACCCAGAGAGAGGGGACGGACGAGACT | |
| | | CCACCTATCCCGATAGACAGCGCCGCGACTTACAAGTTGTTGACC | |
| | | CACGCTCCATGGGCCCCCAATGGGTATTCCACACCTTTGACTACA | |
| | | GACGGGGGCTTTTTGGAAAGGACGCTATTAAGCGAGTGTCAGAAA | |
| | | AACCGACAGATCCTGACTACTTTACAACACCTTACAAAAAACCAAG | |
| | | ATTTTTCCCTCCAACAGCAGGAGAAGAAAAACTGCAAGAAGAAGA | |
| | | CTCCGCTTTACAGGAGAAAAGAAGCCCGCTCTCGTCAGAAGAGGG | |
| | | GCAGACGAGGGCGCAAGTCCTCCAGCAGCAGGTCCTCCAGTCGG | |
| | | AGCTCCAGCAGCAGCAGGAGCTCGGGGAGCAGCTCAGATTCCTC | |
| | | CTCAGGGAAATGTTCAAAACCCAAGCGGGCATACACATGAACCCC | |
| | | CGCGCATTTCAGGAGCTGTAA | |
| ABD34290.1 | DQ186996.1 | ATGGCATGGGGATGGTGGAGATGGCGGCGCCGCTGGCCCGCCA | 199 |
| | | GACGCTGGAGGAGACGCCGTCGCCGGCGCCCCGTACGGAGAAC | |
| | | AAGAGCTCGCCGACCTGCTCGACGCTATAGAAGACGACGAACAGT | |
| | | AAGAACCAGGCGGAGGCGGTGGGGGCGCAGACGGTACAGACGG | |
| | | GGCTGGAGACGCAGGACTTATGTGAGGAAGGGGCGACACAGAAA | |
| | | AAAGAAAAAGAGACTCATACTGAGACAGTGGCAGCCCGCCACCAG | |
| | | ACGCAGATGCACCATAACAGGGTACCTGCCCATAGTGTTCTGCGG | |
| | | CCACACTAAGGGCAATAAAAACTACGCCCTACACTCTGACGACTAC | |
| | | ACCCCCCAAGGACAGCCATTTGGAGGGGCTCTAAGCACTACCTCA | |
| | | TTCTCTTTAAAAGTACTGTTTGACCAGCATCAGAGAGGACTGAATA | |
| | | AGTGGTCGTTCCCCAACGACCAACTAGACCTGGCCAGATACAGGG | |
| | | GCTGCAAATTCTACTTTTACAGGACAAAACAGACTGACTGGATAGG | |
| | | CCAGTATGATATATCAGAGCCCTACAAGCTAGACAAGTACAGCTGC | |
| | | CCCAACTACCACCCGGGAAACATGATTAAAGCAAAGCACAAATTTT | |
| | | TAATTCCCAGCTATGACACTAATCCCAGGGGCAGACAAAAAATTAT | |
| | | AGTTAAAATTCCCCCCCCAGACCTCTTTGTAGACAAGTGGTACACT | |
| | | CAGGAAGACCTGTGTTCCGTTAATCTTGTGTCACTTGCGGTTTCTG | |
| | | CGGCTTCCTTTCTCCACCCATTCGGCTCACCACAAACTGACAACCC | |
| | | TTGCTACACCTTCCAGGTGTTGAAAGAGTTCTACTACCAGGCAATA | |
| | | GGCTTCTCAGCAACAGATCAACAAAGAGAAAAAGTTTTTGATATAT | |
| | | TATACAAAAACAACTCATACTGGGAATCAAACATAACTCCCTTTTAT | |
| | | GTAATTAATGTTAAAAAAGGGTCTAACACAACACAGTACATGTCAC | |
| | | CTCAAATTTCAGACTCATCTTTTAGAAAGAAAGTAAATACTAACTAC | |
| | | AACTGGTATACCTACGATGCCAAAACTAATGCATCACAATTAAAGC | |
| | | AACTAAGAAATGCATACTTTAAACAATTAACCTCTGAAGGCCCACA | |
| | | ACACACATACTCTGACAATGGCTACGCCAGTCAGTGGACCACCCC | |
| | | CAGCACAGACGCCTACGAATACCACTTAGGCATGTTTAGTACTATA | |
| | | TTTTTAGCCCCAGACAGACCAGTACCTCGCTTTCCCTGCGCTTACC | |
| | | AAGATGTTACTTACAACCCACTAATGGACAAAGGAGTGGGCAACC | |
| | | ATGTATGGTTTCAATACAACACAAAGGCAGACACACAGCTAATAGT | |
| | | TACAGGAGGGTCCTGCAAAGCACACATACAAGACATACCCCTATG | |
| | | GGCAGCCTTCTATGGATACAGTGACTTTATAGAGTCAGAGCTAGG | |
| | | CCCCTTTGTAGACGCAGACACAGTAGGCCTTATCTGTGTAATATGC | |
| | | CCTTACACTAAACCTCCCATGTACAACAAGACAAATCCCATGATGG | |
| | | GGTACGTGTTTTATGACAGAAACTTTGGTGACGGCAAATGGACTGA | |
| | | CGGACGGGGCAAAATAGAGCCCTACTGGCAAGTTAGGTGGAGGC | |
| | | CCGAAATGCTTTTCCAAGAAACTGTAATGGCAGACATAGTACAGAC | |
| | | AGGGCCCTTTAGCTACAAAGATGAACTTAAAAACAGCACACTAGTA | |
| | | TGCAAGTACAAATTCTATTTTACCTGGGGAGGTAACATGATGTTCC | |
| | | AACAGACGATCAAAAACCCGTGCAAGACGGACGGACAACCCACC | |
| | | GACTCCAGTAGACACCCTAGAGGAATACAAGTGGCGGACCCGGAA | |
| | | CAAATGGGACCCCGCTGGGTGTTCCACTCCTTTGACTGGCGAAGG | |
| | | GGCTATCTTAGCGAGAAAGCTCTCAAACGCCTGCAAGAAAAACCT | |
| | | CTTGACTATGACGAATATTTTACACAACCAAAAAGACCTAGAATCTT | |
| | | TCCTCCAACAGAATCAGCAGAGGGAGAGTTCCGAGAGCCCGAAAA | |
| | | AGGCTCGTATTCAGAGGAAGAAAGGTCGCAAGCCTCTGCCGAAGA | |
| | | GCAGACGGAGGAGGCGACAGTACTCCTCCTCAAGCGACGACTCA | |
| | | GAGAGCAACAGCAGCTCCAGCAGCAGCTCCAATTCCTCACCCGAG | |
| | | AAATGTTCAAAACGCAAGCGGGTCTCCACATAAACCCTATGTTATT | |
| | | AAACCAGCGATAA | |
| ABD34292.1 | DQ186997.1 | ATGGCATGGGGATGGTGGAGATGGCGGCGCCGCTGGCCCGCCA | 200 |
| | | GACGCTGGAGGAGACGCCGTCGCCGGCGCCCCGTACGGAGAAC | |
| | | AAGAGCTCGCCGACCTGCTCGACGCTATAGAAGACGACGAACAGT | |
| | | AAGAACCAGGCGGAGGCGGTGGGGGCGCAGACGGTACAGACGG | |
| | | GGCTGGAGACGCAGGACTTATGTAAGGAAGGGGCGACACAGAAA | |
| | | AAAGAAAAAGAGACTGATACTGAGACAGTGGCAGCCCGCCACCAG | |
| | | ACGCAGATGCACCATAACAGGGTACCTGCCCATAGTGTTCTGCGG | |
| | | CCACACTAAGGGCAATAAAAACTACGCCCTACACTCTGACGACTAC | |
| | | ACCCCCCAAGGACAGCCATTTGGAGGGGCTCTAAGCACTACCTCA | |
| | | TTCTCTTTAAAAGTACTGTTTGACCAGCATCAGAGAGGACTGAATA | |
| | | AGTGGTCGTTCCCCAACGACCAACTAGACCTGGCCAGATACAGGG | |
| | | GCTGCAAATTCTACTTTTACAGGACAAAACAGACTGACTGGATAGG | |
| | | CCAGTATGATATATCAGAGCCCTACAAGCTAGACAAGTACAGCTGC | |
| | | CCCAACTACCACCCGGGAAACATGATTAAAGCAAAGCACAAATTTT | |
| | | TAATTCCCAGCTATGACACTAATCCCAGGGGCAGACAAAAAATTAT | |
| | | AGTTAAAATTCCCCCCCCAGACCTCTTTGTAGACAAGTGGTACACT | |
| | | CAGGAAGACCTCTGTTCCGTTAATCTTGTGTCACTTGCGGTTTCTG | |
| | | CGGCTTCCTTTCTCCACCCATTCGGCTCACCACAAACTGACAACCC | |
| | | TTGCTACACCTTCCAGGTGTTGAAAGAGTTCTACTACCAGGCAATA | |
| | | GGCTTCTCAGCAACAGATGAACAAAGAGAAAAAGTTTTTGATATAT | |
| | | TATACAAAAACAACTCATACTGGGAATCAAACATAACTCCCTTTTAT | |
| | | GTAATTAATGTTAAAAAAGGGTGTAACACAACACAGTACATGTCAC | |
| | | CTCAAATTTCAGACTCATCTTTTAGAAAGAAAGTAAATACTAACTAC | |
| | | AACTGGTATACCTACGATGCCAAAACTAATGCATCACAATTAAAGC | |
| | | AACTAAGAAATGCATACTTTAAACAATTAACCTCTGAAGGCCCACA | |
| | | ACACACATACTCTGACAATGGCTACGCCAGTCAGTGGACCACCCC | |
| | | CAGCACAGACGCCTACGAATACCACTTAGGCATGTTTAGTACTATA | |
| | | TTTTTAGCCCCAGACAGACCAGTACCTCGCTTTCCCTGCGCTTACC | |
| | | AAGATGTTACTTACAACCCACTAATGGACAAAGGAGTGGGCAACC | |
| | | ATGTATGGTTTCAGTACAACACAAAGGCAGACACACAGCTAATAGT | |
| | | TACAGGAGGGTCCTGCAAAGCACACATACAAGACATACCCCTATG | |
| | | GGCAGCCTTCTATGGATACAGTGACTTTATAGAGTCAGAGCTAGG | |
| | | CCCCTTTGTAGACGCAGACACAGTAGGCCTTATCTGTGTAATATGC | |
| | | CCTTACACTAAACCCCCCATGTACAACAAGACAAATCCCATGATGG | |
| | | GGTACGTGTTTTATGACAGAAACTTTGGTGACGGCAAATGGACTGA | |
| | | CGGACGGGGCAAAATAGAGCCCTACTGGCAAGTTAGGTGGAGGC | |
| | | CCGAAATGCTTTTCCAAGAAACTGTAATGGCAGACATAGTACAGAC | |
| | | AGGGCCCTTTAGCTACAAAGATGAACTTAAAAACAGCACACTAGTA | |
| | | TGCAAGTACAAATTCTATTTTACCTGGGGAGGTAACATGATGTTCC | |
| | | AACAGACGATCAAAAACCCGTGCAAGACGGACGGACAACCCACC | |
| | | GACTCCAGTAGACACCCTAGAGGAATACAAGTGGCGGACCCGGAA | |
| | | CAAATGGGACCCCGCTGGGTGTTCCACTCCTTTGACTGGCGAAGG | |
| | | GGCTATCTTAGCGAGAAAGCTCTCAAACGCCTGCAAGAAAAACCT | |
| | | CTTGACTATGACCAATATTTTACACAACCAAAAAGACCTAGAATCTT | |
| | | TCCTCCAACAGAATCAGCAGAGGGAGAGTTCCGAGAGCCCGAAAA | |
| | | AGGCTCGTATTCAGAGGAAGAAAGGTTGCAAGCCTCTGCCGAAGA | |
| | | GCAGACGGAGGAGGCGACAGTACTCCTCCTCAAGCGACGACTCA | |
| | | GAGAGCAACAGCAGCTCCAGCAGCAGCTCCAATTCCTCACCCGAG | |
| | | AAATGTTCAAAACGCAAGCGGGTCTCCACATAAACCCTATGTTATT | |
| | | AAACCAGCGATAA | |
| ABD34294.1 | DQ186998.1 | ATGGCATGGGGATGGTGGAGATGGCGGCGCCGCTGGCCCGCCA | 201 |
| | | GACGCTGGAGGAGACGCCGTCGCCGGCGCCCCGTACGGAGAAC | |
| | | AAGAGCTCGCCGACCTGCTCGACGCTATAGAAGACGACGAACAGT | |
| | | AAGAACCAGGCGGAGGCGGTGGGGGCGCAGACGGTACAGACGG | |
| | | GGCTGGAGACGCAGGACTTATGTAAGGAAGGGGCGACACAGAAA | |
| | | AAAGAAAAAGAGACTGATACTGAGACAGTGGCAGCCCGCCACCAG | |
| | | ACGCAGATGCACCATAACAGGGTACCTGCCCATAGTGTTCTGCGG | |
| | | CCACACTAAGGGCAATAAAAACTACGCCCTACACTCTGACGACTAC | |
| | | ACCCCCCAAGGACAGCCATTTGGAGGGGCTCTAAGCACTACCTCA | |
| | | TTCTCTTTAAAAGTACTGTTTGACCAGCATCAGAGAGGACTGAATA | |
| | | AGTGGTCGTTCCCCAACGACCAACTAGACCTGGCCAGATACAGGG | |
| | | GCTGCAAATTCTACTTTTACAGGACAAAACAGACTGACTGGATAGG | |
| | | CCAGTATGATATATCAGAGCCCTACAAGCTAGACAAGTACAGCTGC | |
| | | CCCAACTACCACCCGGGAAACATGATTAAAGCAAAGCACAAATTTT | |
| | | TAATTCCCAGCTATGACACTAATCCCAGGGGCAGACAAAAAATTAT | |
| | | AGTTAAAATTCCCCCCCCAGACCTCTTTGTAGACAAGTGGTACACT | |
| | | CAGGAAGACCTGTGTTCCGTTAATCTTGTGTCACTTGCGGTTTCTG | |
| | | CGGCTTCCTTTCTCCACCCATTCGGCTCACCACAAACTGACAACCC | |
| | | TTGCTACACCTTCCAGGTGTTGAAAGAGTTCTACTACCAGGCAATA | |
| | | GGCTTCTCAGCAACAGATGAACAAAGAGAAAAAGTTTTTGATATAT | |
| | | TATACAAAAACAACTCATACTGGGAATCAAACATAACTCCCTTTTAT | |
| | | GTAATTAATGTTAAAAAAGGGTGTAACACAACACAGTGCATGTCAC | |
| | | CTCAAATTTCAGACTCATCTTTTAGAAAGAAAGTAAATACTAACTAC | |
| | | AACTGGTATACCTACGATGCCAAAACTAATGCATCACAATTAAAGC | |
| | | AACTAAGAAATGCATACTTTAAACAATTAACCTCTGAAGGCCCACA | |
| | | ACACACATACTCTGACAATGGCTACGCCAGTCAGTGGACCACCCC | |
| | | CAGCACAGACGCCTACGAATACCACTTAGGCATGTTTAGTACTATA | |
| | | TTTTTAGCCCCAGACAGACCAGTACCTCGCTTTCCCTGCGCGTAC | |
| | | CAAGATGTTACTTACAACCCACTAATGGACAAAGGAGTGGGCAAC | |
| | | CATGTATGGTTTCAGTACAACACAAAGGCAGACACACAGCTAATAG | |
| | | TTACAGGAGGGTCCTGCAAAGCACACATACAAGACATACCCCTAT | |
| | | GGGCAGCCTTCTATGGATACAGTGACTTTATAGAGTCAGAGCTAG | |
| | | GCCCCTTTGTAGACGCAGACACAGTAGGCCTTATCTGTGTAATATG | |
| | | CCCTTACACTAAACCCCCCATGTACAACAAGACAAATCCCATGATG | |
| | | GGGTACGTGTTTTATGACAGAAACTTTGGTGACGGCAAATGGACT | |
| | | GACGGACGGGGCAAAATAGAGCCCTACTGGCAAGTTAGGTGGAG | |
| | | GCCCGAAATGCTTTTCCAAGAAACTGTAATGGCAGACATAGTACAG | |
| | | ACAGGGCCCTTTAGCTACAAAGATGAACTTAAAAACAGCACACTAG | |
| | | TATGCAAGTACAAATTCTATTTTACCTGGGGAGGTAACATGATGTT | |
| | | CCAACAGACGATCAAAAACCCGTGCAAGACGGACGGACAACCCAC | |
| | | CGACTCCAGTAGACACCCTAGAGGAATACAAGTGGCGGACCCGG | |
| | | AGCAAATGGGACCCCGCTGGGTGTTCCACTCCTTTGACTGGCGAA | |
| | | GGGGCTATCTTAGCGAGAAAGCTCTCAAACGCCTGCAAGAAAAAC | |
| | | CTCTTGACTATGACCAATATTTTACACAACCAAAAAGACCTAGAATC | |
| | | TTTCCTCCAACAGAATCAGCAGAGGGAGAGTTCCGAGAGCCCGAA | |
| | | AAAGGCTCGTATTCAGAGGAAGAAAGGTCGCAAGCCTCTGCCGAA | |
| | | GAGCGGACGGAGGAGGCGACAGTACTCCTCCTCAAGCGACGACT | |
| | | CAGAGAGCAACAGCAGCTCCAGCAGCAGCTCCAATTCCTCACCCG | |
| | | AGAAATGTTCAAAACGCAAGCGGGTCTCCACATAAACCCTATGTTA | |
| | | TTAAACCAGCGATAA | |
| ABD34296.1 | DQ186999.1 | ATGGCATGGAGATGGTGGAAGCGACGGAGGCGCTGGTGGTTCCG | 202 |
| | | CAAGCGGTGGACCCGTGGCAGACTTCGCAGACGATGGCCTCGAC | |
| | | CAGCTCGTCGCCGACCTAGACGACGAAGAGTAAGGAGACGCAGA | |
| | | CGTTGGAGGAGGGGGCGACCCAGACGTAGACTGTACCGACGCTA | |
| | | CAGACGCAAAAAACGTAGGAGACGAAAGCCCAAAATAATCTTAAAA | |
| | | CAATGGCAGCCAGACATTGTAAAGAGGTGCTACATAGTGGGCTAC | |
| | | ATTCCTGCCATAATATGTGGGGCGGGCACCTGGTCTCACAACTAC | |
| | | ACCAGCCACCTTCTAGACATTATCCCCAAGGGACCCTTTGGAGGA | |
| | | GGGCACAGCACTATGAGGTTCTCCCTAAAAGTACTCTCTGAAGAA | |
| | | CACCTCAGACACTTAAACTTTTGGACAAAGAGTAACCAGGACCTAG | |
| | | AACTGATAAGATACTTTAGATGCTCCTTTAAATTTTATAGAGACCAA | |
| | | GACACAGACTACATAGTACACTACAGCAGAAAAACTCCCCTGGGA | |
| | | GGCAACAGACTGACAGCACCTAACCTGCACCCAGGGGTACAAATG | |
| | | CTTAGCAAAAACAAAATAATAGTACCTAGCTATGCTACAAAACCCA | |
| | | AGGGTCCTAGCTATATAAAAGTAACCATAGCACCCCCCACACTGCT | |
| | | AACTGACAAGTGGTACTTTAGCAAAGACGTTTGTGACACAACCTTG | |
| | | GTTAACTTAGACGTTGTACTCTGCAACCTGCGGTTTCCGTTCTGCT | |
| | | CACCACAAACTGACAACCCTTGCATCACATTCCAAGTTCTGCATTC | |
| | | CATCTACAACGACTTCCTCTCTATAGTAGATACTAACAACTATAAAG | |
| | | AATCTTTTGTTAGTGCATTACCAACAAAAGTATCTACTGACTGGGG | |
| | | CAAAAGACTAAACACCTTTAGAACAGAAGGATGCTATTCACACCCC | |
| | | AAATTACATAAAAAAGCTGTAACAGCTGCTACAGATACAGAATACTT | |
| | | TACAAAGCCAGATGGTCTGTGGGGAGACACTATATTTGATGTAGAA | |
| | | AATGGACAAAAAATTATAAAAAATATGGAGTCATATGCTAAGTCAG | |
| | | CCAAAGAAAGAGGGATCAATGGAGACCCTGCTTTCTGTCACTTAAC | |
| | | AGGAATATACTCACCTCCCTGGTTAACACCAGGGAGAATATCTCCA | |
| | | GAAACACCTGGACTTTACACAGACGTGACTTACAACCCTTACGCTG | |
| | | ACAAAGGAGTGGGCAACAGAATATGGGTTGACTACTGCAGTAAAA | |
| | | AAGGCAACAAATATGACAATACAAGTAAATGCCTTTTAGAAGACAT | |
| | | GCCACTATGGATGGTATGCTTTGGCTATGTAGACTGTGTAAAAAAA | |
| | | GAAACCGGCAACTGGGGCATTCCACTATGGGCTAGAGTACTTATA | |
| | | AGAAGCCCATATACTGTTCCCAAACTATATAATGAAGCAGACCCAA | |
| | | ACTATGGATGGGTACCTATTTTTTACTATTTTGGAGAAGGCAAAAT | |
| | | GCCAAACGGAGACATGTACATACCATTTAAAATAAGAATGAAATGG | |
| | | TACCCTTCAATGTGGAACCAAGAGCCAGTATTAAATGACTTAGCAA | |
| | | AGAGCGGACCGTTTGCATACAAAAACACCAAAACAAGTGTGACTG | |
| | | TGACTGCCAAATATAAATTCACATTTAACTTCGGTGGCAACCCCGT | |
| | | ACCCTCACAGATTGTACAAGATCCCTGCACACAGCCCACCTACGA | |
| | | CATCCCCGGCACCGGTAACCTGCCTCGCAGAATACAAGTCATTGA | |
| | | CCCGAAAGTCCTCAGTCCCCACTATTCCTTCCACCGGTGGGACTT | |
| | | CAGACGTGGCCTGTTTGGCTCACAAGCTATTAAGAGAGTGTCAGA | |
| | | ACAATCAACAACTTCTGAGTTTTTATTCTCAGGCCCAAAGAAACCC | |
| | | AGAATCGATCAAGGTCCTTACATCCCGCCAGAAAAAGGCTCAGGT | |
| | | TCACTCCAAAGAGAACCGAGACCGTGGAGCAGCTCGGAGACCGA | |
| | | GGCAGAGACAGAAGCCCCCTCGGAAGAAGAGCCGGAGAACCAAG | |
| | | AAGAACAAGTACTCCAGTTGCAGCTCAGACAGCAGCTCCGAGAAC | |
| | | AGCGAAAACTCAGACAGGGAATCCAGTGCCTATTCGAGCAACTAA | |
| | | TAACAACTCAGCAGGGGGTCCACAAAAACCCATTGTTAGAGTAG | |
| ABD34298.1 | DQ187000.1 | ATGGCATGGAGATGGTGGAAGCGACGGAGGCGCTGGTGGTTCCG | 203 |
| | | CAAGCGGTGGACCCGTGGCAGACTTCGCAGACGATGGCCTCGAC | |
| | | CAGCTCGTCGCCGACCTAGACGACGAAGAGTAAGGAGACGCAGA | |
| | | CGTTGGAGGAGGGGGCGACCCAGACGTAGACTGTACCGACGCTA | |
| | | CAGACGCAAAAAACATAGGAGACGAAAGCCCAAAATAATCTTAAAA | |
| | | CAATGGCAGCCAGACATTGTAAAGAGGTGCTACATAGTGGGCTAC | |
| | | ATTCCTGCCATAATATGTGGGGCGGGCACCTGGTCTCACAACTAC | |
| | | ACCAGCCACCTTCTAGACATTATCCCCAAGGGACCCTTTGGAGGA | |
| | | GGGCACAGCACTATGAGGTTCTCCCTAAAAGTACTCTTTGAAGAAC | |
| | | ACCTCAGACACTTAAACTTTTGGACAAAGAGTAACCAGGACCTAGA | |
| | | ACTGATAAGATACTTTAGATGCTCCTTTAAATTTTATAGAGACCAAG | |
| | | ACACAGACTACATAGTACACTACAGCAGAAAAACTCCCCTGGGAG | |
| | | GCAACAGACTGACAGCACCTAACCTGCACCCAGGGGTACAAATGC | |
| | | TTAGCAAAAACAAAATAATGGTACCTAGCTATGCTACAAAACCCAA | |
| | | GGGTCCTAGCTATATAAAAGTAACCATAGCACCCCCCACACTGCTA | |
| | | ACTGACAAGTGGTACTTTAGCAAAGACGTTTGTGACACAACCTTGG | |
| | | TTAACTTAGACGTTGTACTCTGCAACCTGCGGTTTCCGTTCTGCTC | |
| | | ACCACAAACTGACAACCCTTGCATCACATTCCAAGTTCTGCATTCC | |
| | | ATCTACAACGACTTCCTCTCTATAGTAGATACTAACAACTATAAAGA | |
| | | ATCTTTTGTTAGTGCATTACCAACAAAAGTATCTACTGACTGGGGC | |
| | | AAAAGACTAAACACCTTTAGAACAGAAGGATGCTATTCACACCCCA | |
| | | AATTACATAAAAAAGCTGTAACAGCTGCTACAGATACAGAATACTTT | |
| | | ACAAAGCCAGATGGTCTGTGGGGAGACACTATATTTGATGTAGAAA | |
| | | ATGGACAAAAAATTATAAAAAATATGGAGTCATATGCTAAGTCAGC | |
| | | CAAAGAAAGAGGGATCAATGGAGACCCTGCTTTCTGTCACTTAACA | |
| | | GGAATATACTCACCTCCCTGGTTAACACCAGGGAGAATATCTCCAG | |
| | | AAACACCTGGACTTTACACAGACGTGACTTACAACCCTTACGCTGA | |
| | | CAAAGGAGTGGGCAACAGAATATGGGTTGACTACTGCAGTAAAAA | |
| | | AGGCAACAAATATGACAATACAAGTAAATGCCTTTTAGAAGACATG | |
| | | CCACTATGGATGGTATGCTTTGGCTATGTAGACTGGGTAAAAAAAG | |
| | | AAACCGGCAACTGGGGCATTCCACTATGGGCTAGAGTACTTATAA | |
| | | GAAGCCCATATACTGTTCCCAAACTATATAATGAAGCAGACCCAAA | |
| | | CTATGGATGGGTACCTATTTCTTACTATTTTGGAGAAGGCAAAATG | |
| | | CCAAACGGAGACATGTACATACCATTTAAAATAAGAATGAAGTGGT | |
| | | ACCCTTCAATGTGGAACCAAGAGCCAGTATTAAATGACTTAGCAAA | |
| | | GAGCGGACCGTTTGCATACAAAAACACCAAAACAAGTGTGACTGT | |
| | | GACTGCCAAATATAAATTCACATTTAACTTCGGTGGCAACCCCGTA | |
| | | CCCTCACAGATTGTACAAGATCCCTGCACACAGCCCACCTACGAC | |
| | | ATCCCCGGCACCGGTAACCTGCCTCGCAGAATACAAGTCATTGAC | |
| | | CCGAAAGTCCTCGGTCCCCACTATTCCTTCCACCGGTGGGACTTC | |
| | | AGACGTGGCCTGTTTGGCTCACAAGCTATTAAGAGAGTGTCAGAA | |
| | | CAATCAACAACTTCTGAGTTTTTATTCTCAGGCCCAAAGAAACCCA | |
| | | GAATCGATCAAGGTCCTTACATCCCGCCAGAAAAAGGCTCAGGTT | |
| | | CACTCCAAAGAGAACCGAGACCGTGGAGCAGCTCGGAGACCGAG | |
| | | GCAGAGACAGAAGCCCCCTCGGAAGAAGAGCCGGAGAACCAAGA | |
| | | AGAACAAGTACTCCAGTTGCAGCTCAGACAGCAGCTCCGAGAACA | |
| | | GCGAAAACTCAGACAGGGAATCCAGTGCCTATTCGAGCAACTAAT | |
| | | AACAACTCAGCAGGGGGTCCACAAAAACCCATTGTTAGAGTAG | |
| ABD34300.1 | DQ187001.1 | ATGGCACGGAGATGGTGGAAGCGACGGAGGCGCTGGTGGTTCCG | 204 |
| | | CAAGCGGTGGACCCGTGGCAGACTTCGCAGACGATGGCCTCGAC | |
| | | CAGCTCGTCGCCGACCTAAACGACGAAGAGTAAGGAGACGCAGA | |
| | | CGTTGGAGGAGGGGGCGACCCAGACGTAGACTGTACCGACGCTA | |
| | | CAGACGCAAAAAACGTAGGAGACGAAAGCCCAAAATAATCTTAAAA | |
| | | CAATGGCAGCCAGACATTGTAAAGAGGTGCTACATAGTGGACTAC | |
| | | ATTCCTGCCATAATATGTGGGGCGGGCACCTGGTCTCGCAACTAC | |
| | | ACCAGCCACCTTCTAGACATTATCCCCAAGGGACCCTTTGGAGGA | |
| | | GGGCACAGCACTATGAGGTTCTCCCTAAAAGTACTCTTTGAAGAAC | |
| | | ACCTCAGGCACTTAAACTTTTGGACAAAGAGTAACCAGGACCTAGA | |
| | | ACTGATAAGATACTTTAGATGCTCCTTTAAATTTTATAGAGACCAAG | |
| | | ACACAGACCACATAGTACACTACAGCAGAAAAACTCCCCTGGGAG | |
| | | GCAACAGACTGACAGCACCTAACCTGCACCCAGGGGTACAAATGC | |
| | | TTAGCAAAAACAAAATAATAGTACCTAGCTATGCTACAAAACCCAA | |
| | | GGGTCCTAGCTATATAAAAGTAACCATAGCACCCCCCACACTGCTA | |
| | | ACTGACAAGTGGTACTTTAGCAAAGACGTTTGTGACACAACCTTGG | |
| | | TTAACTTAGACGTTGTACTCTGCAACCTGCGGTTTCCGTTCTGCTC | |
| | | ACCACAAACTGACAACCCTTGCATCACATTCCAAGTTCTGCATTCC | |
| | | ATCTACAACGACTTCCTCTCTATAGTAGATACTAACAACTATAAAGA | |
| | | ATCTTTTGTTGCTGCATTACCAACAAAAGTATCTACTGACTGGGGC | |
| | | AAAAGACTAAACACCTTTAGAACAGAGGGATGCTATTCACACCCCA | |
| | | AATTACATAAAAAAGCTGTAACAGCTGCTACAGATACAGAATACTTT | |
| | | ACAAAGCCAGATGGTCTGTGGGGAGACACTATATTTGATGTAGAAA | |
| | | ATGGACAAAAAATTATAAAAAATATGGAATCATATGCTAAGTCAGC | |
| | | CAAAGAAAGAGGGATCAATGGAGACCCTGCTTTCTGTCACTTAACA | |
| | | GGAATATACTCACCTCCCTGGTTAACACCAGGGAGAATATCTCCAG | |
| | | AAACACCTGGACTTTACACAGACGTGACTTACAACCCTTACGCTGA | |
| | | CAAAGGAGTGGGCAACAGAATATGGGTTGACTACTGCAGTAAAAA | |
| | | AGGCAACAAATATGGCAATACAAGTAAATGCCTTTTAGAAGACATG | |
| | | CCACTATGGATGGTATGCTTTGGCTATGTAGACTGGGTAAAAAAAG | |
| | | AAACCGGCAACTGGGGCATTCCACTATGGGCTAGAGTACTTATAA | |
| | | GAAGCCCATATACTGTTCCCAAACTATATAATGAAGCAGACCCAAA | |
| | | CTATGGATGGGTACCTATTTCTTACTATTTTGGAGAAGGCAAAATG | |
| | | CCAAACGGAGACATGTACGTACCATTTAAAATAAGAATGAAATGGT | |
| | | ACCCTTCAATGTGGAACCAAGAGCCAGTATTAAATGACTTAGCAAA | |
| | | GAGCGGACCGTTTGCATACAAAAACACCAAAACAAGTGTGACTGT | |
| | | GACTGCCAAATATAAATTCACATTTAACTTCGGGGGCAACCCCGTA | |
| | | CCCTCACAGATTGTACAAGATCCCTGCACACAGCCCACCTACGAC | |
| | | ATCCCCGGCACCGGTAACCTGCCTCGCAGAATACAAGTCATTGAC | |
| | | CCGAAAGTCCTCGGTCCCCACTATTCCTTCCACCGGTGGGACTTC | |
| | | AGACGTGGCCTGTTTGGCTCACAAGCTATTAAGAGAGTGTCAGAA | |
| | | CAATCAACAACTTCTGAGTTTTTATTCTCAGGCCCAAAGAAACCCA | |
| | | GAATCGATCAAGGTCCTTACATCCCGCCAGAAAAAGGCTCAGGTT | |
| | | CACTCCAAAGAGAACCGAGACCGTGGAGCAGCTCGGAGACCGAG | |
| | | GCAGAGACAGAAGCCCCCTCGGAAGAAGAGCCGGAGAACCAAGA | |
| | | AGAACAAGTACTCCAGTTGCAGCTCAGACAGCAGCTCCGAGAACA | |
| | | GCGAAAACTCAGACAGGGAATCCAGTGCCTATTCGAGCAACTAAT | |
| | | AACAACTCAGCAGGGGGTCCACAAAAACCCATTGTTAGAGTAG | |
| ABD34302.1 | DQ187002.1 | ATGGCATGGAGATGGTGGAAGCGACGGAGGCGCTGGTGGTTCCG | 205 |
| | | CAAGCGGTGGACCCGTGGCAGACTTCGCAGACGATGGCCTCGAC | |
| | | CAGCTCGTCGCCGACCTAAACGACGAAGAGTAAGGAGACGCAGA | |
| | | CGTTGGAGGAGGGAGCGACCCAGACGTAGACTGTACCGACGCTA | |
| | | CAGACGCAAAAAACGTAGGAGACGAAAGCCCAAAATAATCTTAAAA | |
| | | CAATGGCAGCCAGACATTGTAAAGAGGTGCTACATAGTGGGCTAC | |
| | | ATTCCTGCCATAATATGTGGGGCGGGCACCTGGTCTCACAACTAC | |
| | | ACCAGCCACCTTCTAGACATTATCCCCAAGGGACCCTTTGGAGGA | |
| | | GGGCACAGCACTATGAGGTTCTCCCTAAAAGTACTCTTTGAAGAAC | |
| | | ACCTCAGGCACTTAAACTTTTGGACAAAGAGTAACCAGGACCTAGA | |
| | | ACTGATAAGATACTTTAGATGCTCCTTTAAATTTTATAGAGACCAAG | |
| | | ACACAGACTACATAGTACACTACAGCAGAAAAACTCCCCTGGGAG | |
| | | GCAACAGACTGACAGCACCTAACCTGCACCCAGGGGTACAAATGC | |
| | | TTAGCAAAAACAAAATAATAGTACCTAGCTATGCTACAAAACCCAA | |
| | | GGGTCCTAGCTATATAAAAGTAACCATAGCACCCCCCACACTGCTA | |
| | | ACTGACAAGTGGTACTTTAGCAAAGACGTTTGTGACACAACCTTGG | |
| | | TTAACTTAGACGTTGTACTCTGCAAGCTGCGGTTTCCGTTCTGCTC | |
| | | ACCACAAACTGACAACCCTTGCATCACATTCCAAGTTCTGCATTCC | |
| | | ATCTACAACGACTTCCTCTCTATAGTAGATACTAACAACTATAAAGA | |
| | | ATCTTTTGTTGCTGCATTACCAACAAAAGTATCTACTGACTGGGGC | |
| | | AAAAGACTAAACACCTTTAGAACAGAAGGATGCTATTCACACCCCA | |
| | | AATTACATAAAAAAGCTGTAACAGCTGCTACAGATACAGAATACTTT | |
| | | ACAAAGCCAGATGGTCTGTGGGGAGACACTATATTTGATGTAGAAA | |
| | | ATGGACAAAAAATTATAAAAAATATGGAATCATATGCTAAGTCAGC | |
| | | CAAAGAAAGAGGGATCAATGGAGACCCTGCTTTCTGTCACTTAACA | |
| | | GGAATATACTCACCTCCCTGGTTAACACCAGGGAGAATATCTCCAG | |
| | | AAACACCTGGACTTTACACAGACGTGACTTACAACCCTTACGCTGA | |
| | | CAAAGGAGTGGGCGACAGAATATGGGTTGACTACTGCAGTAAAAA | |
| | | AGGCAACAAATATGACAATACAAGTAAATGCCTTTTAGAAGACATG | |
| | | CCACTATGGATGGTATGCTTTGGCTATGTAGACTGGGTAAAAAAAG | |
| | | AAACCGGCAACTGGGGCATTCCACTATGGGCTAGAGTACTTATAA | |
| | | GAAGCCCATATACTGTTCCCAAACTATATAATGAAGCAGACCCAAA | |
| | | CTATGGATGGGTACCTATTTCTTACTATTTTGGAGAAGGCAAAATG | |
| | | CCAAACGGAGACATGTACGTACCATTTAAAATAAGAATGAAATGGT | |
| | | ACCCTTCAATGTGGAACCAAGAGCCAGTATTAAATGACTTAGCAAA | |
| | | GAGCGGACCGTTTGCATACAAAAACACCAAAACAAGTGTGACTGT | |
| | | GACTGCCAAATATAAATTCACATTTAACTTCGGGGGCAACCCCGTA | |
| | | CCCTCACAGATTGTACAAAATCCCTGCACACAGCCCACCTACGAC | |
| | | ATCCCCGGCACCGGTAACCTGCCTCGCAGAACACAAGTCATTGAC | |
| | | CCGAAAGTCCTCGGTCCCCACTATTCCTTCCACCGGTGGGACTTC | |
| | | AGGCGCGGCCTGTTTGGCTCACAAGCTATTAAGAGAGTGTCAGAA | |
| | | CAATCAACAACTTCTGAGTTTTTATTCTCAGGCCCAAAGAAACCCA | |
| | | GAATCGATCAAGGTCCTTACATCCCGCCAGAAAAAGGCTCAGGTT | |
| | | CACTCCAAAGAGAACCGAGACCGTGGAGCAGCTCGGAGACCGAG | |
| | | GCAGAGACAGAAGCCCCCTCGGAAGAAGAGCCGGAGAACCAAGA | |
| | | AGAACAAGTACTCCAGTTGCAGCTCAGACAGCAGCTCCGAGAACA | |
| | | GCGAAAACTCAGACAGGGAATCCAGTGCCTATTCGAGCAACTAAT | |
| | | AACAACTCAGCAGGGGGTCCACAAAAACCCATTGTTAGAGTAG | |
| ABD34305.1 | DQ187004.1 | ATGGCCTGGGGATGGTGGAAACGCAGACGGCGCCGATGGTGGAG | 206 |
| | | AGGCCTCTGGAGGAGACGCCGCTTTGCCAGAAGACGACCTAGAC | |
| | | GGCCTGCTCGCCGCCCTAGACGACGAAGAGTAAGGAGACGCAGA | |
| | | CGGTGGAGGAGGGGGCGACTAAGGAGGCGCGTGTACAACAGGA | |
| | | GACGCAGGATCAGACGAAAGAGACGCAGACAGAAACTGACAATAA | |
| | | GACAGTGGCAGCCTGACAAACGCAGGATATGTAGAATTAAAGGCT | |
| | | ACCTTCCTGCCATTATATATGGAGACGGGACGTTTTCTAAAAACTA | |
| | | TACAAGTCACTTAGAGGACAGAATCTCCAAAGGACCGTTTGGGGG | |
| | | AGGGCACGGGACTGCTAGAATGTCTCTTAAAGTACTGTATGACGA | |
| | | CCACCTAAAAGGACTTAACATATGGACGTATAGTAACAAGGACTTG | |
| | | GAACTGGTCAGATACATGCACACCACAATTACATTTTACAGACACC | |
| | | CAGACACAGACTTTATAGCAGTATACAACAGAAAAACACCACTAGG | |
| | | TGGCAACAGATACACAGCACCCTCACTGCACCCTGGTAACATGAT | |
| | | GCTGCAGAGAACTAAAATACTAATCCCTAGCTTTAAAACCAAACCC | |
| | | AGAGGGAGCGGCAAAATTAGAGTAGTAATAAAACCCCCCACTCTG | |
| | | TTAGTAGATAAGTGGTACTTTCAAAAGGACATATGCGACGTTACAC | |
| | | TGTTTAACCTCAACATTACAGCAGCTAGCCTGCGGTTTCCGTTCTG | |
| | | CTCACCACAAACGAACAACCCTTGTGTAACATTCCAAGTTCTGCAT | |
| | | TCTGTGTATGACAAAGCATTAGGCATTAACACATTTGGTACCAAAG | |
| | | AAACACCAGAAGATCAGCAAATGGAAGATATTAAAAACTGGCTTAC | |
| | | CAAAGCTCTAAATACTGCAGGCTTTACTGTACTAAATACATTTAGAA | |
| | | CAGAAGGTATATACTCACACCCACAACTAAAAAAACCACCTGAAGG | |
| | | AAGTAACAAACCTAGTGCAGAACAGTACTTTGCTCCACTAGACAGC | |
| | | TTATGGGGAGACAAGATATATGTAAATAATAATACTAGTCCTTCACA | |
| | | AACAGAAGCAACAATTCCAGGTATATTAGCCAGAAATGCTTGCACA | |
| | | TACTATCAAAAAGCTAAAACAAGCACACTAAGGCAGCACCTAGGC | |
| | | GCTATGGCACACTGTCACCTAACAGGAATTTTTAACCCTGCACTAC | |
| | | TAACACAGGGCAGACTATCACCAGAATTTTTTGGCCTATACAAAGA | |
| | | AATTATTTATAACCCCTATGATGACAAAGGCAAAGGAAACAGAATA | |
| | | TGGATAGACCCATTAACAAAACCTGACAACATATTTGATGCTAGAA | |
| | | GTAAAGTAGAACTAGAAGATATGCCTCTTTGGATGGCATGCTTTGG | |
| | | ATATAATGACTGGTGTAAAAAAGAGCTAAATAACTGGGGCCTAGAA | |
| | | GTAGAATACAGAGTACTACTAAGATGCCCTTACACATATCCAAAAC | |
| | | TGTACAATGATGCTAACCCAAACTATGGCTATGTACCTATATCCTA | |
| | | CAACTTTAGTGCAGGAAAAACTGTAGAAGGGGATCTTTATGTTCCA | |
| | | ATAATGTGGAGAACTAAATGGCATCCAACAATGTACAATCAATCTC | |
| | | CAGTACTAGAAGATTTAGCCATGGCAGGGCCTTTTGCTCCAAAAGA | |
| | | AAAAATACCTAGCAGCACACTTACAATAAAATACAAAGCTAAATTTA | |
| | | TATTCGGGGGCAATCCTATATCTGAACAGATTGTCAAGGACCCCTG | |
| | | CACCCAGCCCACCTACGAAATTCCCGGAGGCGGTACGCTCCCTC | |
| | | GCAGAATACAAGTCATTAACCCGGAATACATCGGGCCACACTACT | |
| | | CATTCAAAAGCTTCGACATCAGACGTGGGTACTTTAGCGCGAAGA | |
| | | GTGTTAAAAGAGTGTCAGAACAATCAGACATTACTGAGTTTATATTC | |
| | | TCAGGTCCAAAAAAGCCAAGGATCGACCAAGACAGGTACCAAGAA | |
| | | GCAGAAGAACACTCAGATTCTCGACTCCGAGAAGAGAAACCGTGG | |
| | | GAGAGCTCGCAAGAAACAGAGAGCGAAGCCCAAGAAGAAGAGAT | |
| | | ACAAGAGACAAACATCCAGCTCCAGCTGCAGCACCAGCTCAAAGA | |
| | | GCAACTGCAGCTCAGACGGGGAATCCAGTGCCTCTTCGAGCAACT | |
| | | AACCAAAACCCAGCAGGGAGTCCACATAAACCCTTCCCTCGTGTA | |
| | | G | |
| ABD34307.1 | DQ187005.1 | ATGTCTCTTAAAGTACTGTATGACGACCACCTAAAAGGACTTAACA | 207 |
| | | TATGGACGTATAGTAACAAGGACTTGGAACTGGTCAGATACATGCA | |
| | | CACCACAATTACATTTTACAGACACCCAGACACAGACTTTATAGCA | |
| | | GTATACAACAGAAAAACACCACTAGGTGGCAACAGATACACAGCA | |
| | | CCCTCACTGCACCCTGGTAACATGATGCTGCAGAGAACTAAAATAC | |
| | | TAATCCCTAGCTTTAAAACCAAACCCAGAGGGAGCGGCAAAATTAG | |
| | | AGTAGTAATAAAACCCCCCACTCTGTTAGTAGATAAGTGGTACTTT | |
| | | CAAAAGGACATATGCGACGTTACACTGTTTAACCTCAACATTACAG | |
| | | CAGCTAGCCTGCGGTTTCCGTTCTGCTCACCACAAACGAACAACC | |
| | | CTTGTGTAACATTCCAAGTTCTGCATTCTGTGTATGACAAAGCATTA | |
| | | GGCATTAACACATTTGGTACCAAAGAAACACCAGAAGATCAGCAAA | |
| | | TGGAAGATATTAAAAACTGGCTTACCAAAGCTCTAAATACTGCAGG | |
| | | CTTTACTGTACTAAATACATTTAGAACAGAAGGTATATACTCACACC | |
| | | CACAACTAAAAAAACCACCTGAAGGAAGTAACAAACCTAGTGCAGA | |
| | | ACAGTACTTTGCTCCACTAGACAGCTTATGGGGAGACAAGATATAT | |
| | | GTAAATAATAATACTAGTCCTTCACAAACAGAAGCAACAATTCCAG | |
| | | GTATACTAGCCAGAAATGCTTGCACATACTATCAAAAAGCTAAAAC | |
| | | AAGCACACTAAGGCAGCACCTAGGCGCTATGGCACACTGTCACCT | |
| | | AACAGGAATTTTTAACCCTGCACTACTAACACAGGGCAGACTATCA | |
| | | CCAGAATTTTTTGGCCTATACAAAGAAATTATTTATAACCCCTATGA | |
| | | TGACAAAGGCAAAGGAAACAGAATATGGATAGACCCATTAACAAAA | |
| | | CCTGACAACATATTTGATGCTAGAAGTAAAGTAGAACTAGAAGATA | |
| | | TGCCTCTTTGGATGGCATGCTTTGGATATAATGACTGGTGTAAAAA | |
| | | AGAGCTAAATAACTGGGGCCTAGAAGTAGAATACAGAGTACTACTA | |
| | | AGATGCCCTTACACATATCCAAAACTGTACAATGATGCTAACCCAA | |
| | | ACTATGGCTATGTACCTATATCCTACAACTTTAGTGCAGGAAAAAC | |
| | | TGTAGAAGGGGATCTTTATGTTCCAATAATGTGGAGAACTAAATGG | |
| | | TATCCAACAATGTACGATCAATCTCCAGTACTAGAAGATTTAGCCA | |
| | | TGGCAGGGCCTTTTGCTCCAAAAGAAAAAATACCTAGCAGCACACT | |
| | | TACAATAAAATACAAAGCTAAATTTATATTCGGGGCAATCCTATATC | |
| | | TGAACAGATTGTCAAGGACCCCTGCACCCAGCCCACCTACGAAAT | |
| | | TCCCGGAGGCGGTACGCTCCCTCGCAGAATACAAGTCATTAACCC | |
| | | GGAATACATCGGGCCACACTACTCATTCAAAAGCTTCGACATCAGA | |
| | | CGTGGGTACTTTAGCGCGAAGAGTGTTAAAAGAGTGTCAGAACAA | |
| | | TCAGACATTACTGAGTTTATATTCTCAGGTCCAAAAAAGCCAAGGA | |
| | | TCGACCAAGACAGGTACCAAGAAGCAGAAGAACACTCAGATTCTC | |
| | | GACTCCGAGAAGAGAAACCGTGGGAGAGCTCGCAAGAAACAGAG | |
| | | AGCGAAGCCCAAGAAGAAGAGATACAAGAGACAAACATCCAGCTC | |
| | | CAGCTGCAGCACCAGCTCAAAGAGCAACTGCAGCTCAGACGGGG | |
| | | AATCCAGTGCCTCTTCGAGCAACTAA | |
| ABD61942.1 | DQ361268.1 | ATGGCCTGGAGATGGTGGTGGAGACGCAGGCGCCCGTGGCGATG | 208 |
| | | GAGATGGAGGCGAAGGAGACGACCAGCTAGACGCCGAAGACGTA | |
| | | GAAGACCTGCTCGGCGTGCTAGACGACCCAGAGTAAGGAGATGG | |
| | | CGCAGGCGCAGGGTGTGGGCGCCCAGGCCATACATAAGAAGGCG | |
| | | CAGGCGAAGCTTCCGTAGAAAAAAAATTAAAATAACTCAGTGGAAC | |
| | | CCCGCTGTTACTAAAAAATGTACTGTAACTGGGTACCTACCAGTTA | |
| | | TATACTGTGGAACCGGGGACATAGGAACCACTTTTCAGAACTTTGG | |
| | | CTCTCATATGAATGAGTACAAACAGTATAACGCTGCGGGAGGGGG | |
| | | CTTTAGCACAATGCTTTTTACCATGCAAAACCTGTATGAAGAGTAC | |
| | | CAAAAACATAGATGCAGATGGTCTAAAAGCAATCAAGACCTAGACC | |
| | | TGTGTAGATATCTAGACTGTAAACTAACATTTTACAGATCCCCTAAC | |
| | | ACAGACTTTATAGTTGGCTACAATAGAAAGCCTCCCTTTATAGACA | |
| | | CTCAAATAACAAGATGTACTTTACATCCAGGAATGCTAATACAAGA | |
| | | AAGAAAAAAAGTAATAATACCTAGCTTCCAAACCAGGCCAAAAGGT | |
| | | AGAATAAAACGCAAAATTAAAGTAAGGCCCCCCACCTTATTCACAG | |
| | | ACAAATGGTACTTTCAGAGAGACCTCTGTAAAGTTCCTCTTGTAAC | |
| | | GGTTTCCGCTTCTGCGGCGAGCCTGCGGTTTCCGTTCGGCTCACC | |
| | | ACAAACAGAAAACTATTGCATATACTTCCAGGTTTTAGATCCCTGG | |
| | | TACCACACCCGCCTGAGCATAACTGGTGGAAAGCCAGCTGAATAT | |
| | | TGGACACAGCTAAAAGCTTATTTAACTCAAGGCTGGGGCAGGTCA | |
| | | ACAAATAATGCAGGATATCAACATGGTCCACTAGGTACTTACTTTA | |
| | | ATACACTTAAAACATCAGAACATATTAGACAACCCCCAGCAGATAA | |
| | | CTACAAACAAGCAAATAAAGATACTACATACTATGGAAGAGTAGAC | |
| | | AGTCACTGGGGAGATCATGTATACCAACAAACAATAATACAAGCCA | |
| | | TGGAAGAAAACCAAAGCAACATGTACACAAAAAGAGCACTTCACAC | |
| | | ATTCTTAGGCAGTCAATATCTAAACTTTAAATCAGGTCTATTTAGCA | |
| | | GTATATTTCTAGATAATGCCAGACTAAGCCCAGACTTTAAAGGTAT | |
| | | GTACCAAGAAGTTGTTTATAACCCCTTTAATGACAGAGGAGTAGGC | |
| | | AACAAAGTATGGGTTCAGTGGTGCACAAACGAGGACACAATATTTA | |
| | | AAGACCTACCAGGCAGAGTTCCTGTGGTAGATTTACCATTGTGGT | |
| | | GCGCGTTAATGGGCTACTCAGACTACTGCAAAAAATATTTCCACGA | |
| | | CGATGGCTTCTTAAAAGAGGCCAGAATAACTATAATCAGCCCATAC | |
| | | ACAAATCCTCCACTAATTAACAACAAAAATACAAATGAGGGCTTTGT | |
| | | ACCCTACAGTTTCTACTTTGGAAAAGGCAGAATGCCAGACGGCAAT | |
| | | GGGTACATACCCATAGACTTTAGATTTAACTGGTACCCTTGCATAT | |
| | | TTCACCAAACAAACTGGATAAATGACATGGTTCAATGCGGACCCTT | |
| | | TGCCTACCACGGAGATGAAAAGAACTGTTCTCTCACTATGAAATAC | |
| | | AAGTTTAAATTTCTATTTGGGGGCAATCCTATCTCACAACAGACTAT | |
| | | CAAAGACCCTTGCCAACAACCCGACTGGCAACTTCCCGGTTCCGG | |
| | | TAGATTCCCTCGCGATGTACAAGTATCGAACCCGCGCTTGCAAAC | |
| | | CGAAGGGTCCACGTTCCACGCGTGGGACTTCAGACGGGGTTTCTA | |
| | | TGGCAAAAGAGCTATTGAAAGACTGCAGGGACAACAAGATGATGT | |
| | | TACATATATTGCAGGACCTCCAAAAAGGCCCCGCTTCGAGGTCCC | |
| | | AGCCCTGGCTGCCGAAGGAAGCTCAAATACACGCCGATCAGAGTT | |
| | | GCCATGGCAAACCTCAGAAGAAGAAAGCTCGCAAGAAGAAAACTC | |
| | | AGAAGAGACAGAAGAAGAAACCTCGTTATCGCAGCAGCTCAAGCA | |
| | | GCAGTGCATCGAGCAGAAGCTCCTCAAGCGAACGCTCCACCAACT | |
| | | CGTCAAGCAATTAGTAAAGACCCAGTATCACCTACACGCCCCCATT | |
| | | ATCCACTAA | |
| ABU55887.1 | EF538879.1 | ATGGCATGGAGATGGTGGAAGCGACGGAGGCGCTGGTGGTTCCG | 209 |
| | | CAAGCGGTGGACCCGTGGCAGACTTCGCAGACGATGGCCTCGAC | |
| | | CAGCTCGTCGCCGCCCTAGACGACGAAGAGTAAGGAGACGCAGA | |
| | | CGGTGGAGGAGGGGGCGACCCAGACGCAGACTGTACCGACGCTA | |
| | | CAGACGCAAAAAACGTAGGAGACGAAAGCCCAAAATAATCTTAAAA | |
| | | CAATGGCAGCCAGACATTGTAAAAAGATGCTATATAATAGGCTACA | |
| | | TTCCTGCCATAATATGTGGGGCTGGCACCTGGTCCCACAACTACA | |
| | | CCAGCCACCTGTTAGACATTATCCCCAAGGGACCCTTTGGAGGAG | |
| | | GGCACAGCACTATGAGATTCTCCCTAAAAGTACTCTTTGAAGAACA | |
| | | CCTCAGACACTTAAACTTTTGGACAAAAAGCAACCAGGACCTAGAA | |
| | | CTTATAAGATACTTTAGATGCTCCTTTAAATTCTATAGAGACCAAGA | |
| | | CACAGACTACATAGTACACTACAGCAGAAAGACTCCCCTAGGAGG | |
| | | CAACAGACTGACAGCACCTAGCCTACACCCCGGGGTACAGATGCT | |
| | | TAGCAAAAACAAAATATTAGTACCTAGCTATGCTACAAAACCCAAG | |
| | | GGTGGTAGCTATGTAAAAGTAACCATAGCACCCCCCACACTACTAA | |
| | | CTGACAAGTGGTACTTTAGCAAAGACGTTTGTGACACAACCTTGGT | |
| | | TAACTTAGACGTCGTACTCTGCAACTTGCGGTTTCCGTTCTGCTCA | |
| | | CCACAAACTGACAACCCTTGCATCACATTCCAAGTTCTGCATTCTT | |
| | | ACTACAACGACTACCTCTCTATAGTAGACACCGCCTTATACAAAAC | |
| | | CAGCTTTGTAAACAATTTAAGTACAAAACTAGGTACAACATGGGCA | |
| | | AACAGACTAAACACATTTAGAACAGAAGGCTGCTACTCACATCCAA | |
| | | AATTGCTCAAAAAAACAGTAACAGCTGCAAATGACACCAAATATTTT | |
| | | ACTACACCAGACGGACTCTGGGGAGATGCAGTATTTGATGTTTCA | |
| | | GACGCAAAAAAACTAACTAAAAACATGGAAAGTTATGCTGCCTCTG | |
| | | CTAACGAAAGAGGCGTACAAGGAGACCCTGCCTTTTGCCACCTAA | |
| | | CAGGCATATTCTCACCTCCCTGGCTAACACCAGGCAGAATATCTCC | |
| | | TGAAACCCCAGGACTTTACACAGACGTGACTTACAACCCATACGCA | |
| | | GACAAAGGAGTGGGCAACAGAATATGGGTTGACTACTGTAGTAAA | |
| | | AAAGGCAATAAATATGACAATACAAGTAAATGCGTGTTAGAAGACA | |
| | | TGCCACTATGGATGTTATGCTTTGGCTATGTAGACTGGGTAAAAAA | |
| | | AGAGACTGGCAACTGGGGCATTCCACTATGGGCCAGAGTACTTAT | |
| | | AAGAAGCCCATATACTGTCCCAAAACTATACCATGAAAACGACCCT | |
| | | GACTACGGATGGGTTCCAATTTCCTACTACTTTGGAGAAGGCAAAA | |
| | | TGCCAAACGGAGACATGTACGTACCATTTAAAGTAAGAATGAAATG | |
| | | GTACCCTTCAATGTGGAACCAAGAGCCAGTTTTAAATGACTTAGCA | |
| | | AAGAGCGGACCGTTTGCATACAAGAACACCAAAACAAGCGTGACT | |
| | | GTGACTGCCAAATATAAATTCACATTTAACTTCGGGGGCAACCCCG | |
| | | TACCCTCACAGATTGTACAAGATCCCTGCACACAGCCCACCTACG | |
| | | ACATCCCCGGCACCGGTAACCTGCCTCGCAGAATACAAGTCATTG | |
| | | ACCCGAAAGTCCTCGGTCCCCACTATTCCTTCCACCGGTGGGACT | |
| | | TCAGGCGTGGCCTCTTTGGCACACAAGCTATTAAAAGAGTGTCAG | |
| | | AACAATCAACAACTTCTGAGTTTTTATTCTCAGGCCCAAAGAAACC | |
| | | CAGAATCGATCAAGGCCCTTACATCCCGCCAGAAAAAGGCTCAGG | |
| | | TTCACTCCAAAGAGAATCGAGACCGTGGAGCAGCTCGGAGACCGA | |
| | | GGCAGAGACAGAAGCCCCCTCGGAAGAGGAGCCGGAGAACCAAG | |
| | | AAGAACAAGTACTCCAGTTGCAGCTCAGACAGCAGCTCCGAGAAC | |
| | | AGCGAAAACTCAGACAGGGAATCCAGTGCCTATTCGAGCAACTGA | |
| | | TAACAACCCAGCAGGGGGTCCACAAAAACCCATTGTTAGAGTAG | |
| ABY26045.1 | EU305675.1 | ATGGCCTGGTGGGGACGGTGGAGAAGATGGCGCTGGAGGCCCC | 210 |
| | | GTCGCTGGCGGCGCCGTCGCAGACGCCGAGTACCAAGAAGAAGA | |
| | | GCTCAACGCTCTGTTCGACGCCGTCGAGCAAGAAGAGTAAGGAG | |
| | | GAGGCGATGGGGGAGGCGGAGGTGGAGACGGGGGTACAGACGC | |
| | | AGACTGAGACTAAGACGCAAACGCAAACGAAAACGCAGACTTGTA | |
| | | CTGACTCAGTGGCACCCCGCTAAAGTAAGGAGGTGCAGAATATCT | |
| | | GGGGTCCTACCCATGATACTGTGCGGTGCTGGCAGGAGTAGCTTT | |
| | | AACTACGGGCTGCACAGCGATGACTTTACTAAACAGAAACCAAACA | |
| | | ATCAGAACCCGCACGGCGGGGGCATGAGCACTGTGACTTTTAACC | |
| | | TAAAGGTGCTCTTTGACCAATACGAAAGATTTATGAACAAGTGGTC | |
| | | GTACCCCAACGACCAACTAGACCTCGCCAGATACAAAGGCTGTAA | |
| | | ATTCACCTTCTACAGACACCCAGAAGTTGACTTTCTAGCTCAATAT | |
| | | GACAACGTTCCCCCTATGAAAATGGACGAACTGACTGCCCCTAAC | |
| | | ACTCACCCCGCACTGCTGCTACAGAGCAGACACAGGGTAAAGATA | |
| | | TACAGCTGGAAAACCAGGCCATTTGGCTCTAAAAAAGTAACAGTAA | |
| | | AAATAGGACCCCCCAAACTGTTTGAAGACAAGTGGTACAGCCAGT | |
| | | CTGACTTGTGCAAAGTTTCCCTTGTCAGTTGGCGGTTAACCGCATG | |
| | | TGACTTCAGGTTTCCGTTCTGCTCACCACAAACTGACAACCCTTGT | |
| | | GTAACCTTCCAGGTGCTAGGAGAACAGTATTACGAAGTCTTTGGAA | |
| | | CTTCCGTATTGGACGTTCCTGCATCCTATAACTCACAAATAACTAC | |
| | | ATTTGAACAATGGCTATATAAAAAATGCACCCACTATCAAACATTCG | |
| | | CCACAGACACCAGATTAGCCCCCCAAAAGAAAGCAACCACATCCA | |
| | | CCAACCACACATATAACCCCAGTGGCAACACTGAATCATCAACATG | |
| | | GACACAAAGTAACTACTCCAAATTTAAACCAGGCAACACAGACAGC | |
| | | AACTATGGCTACTGCAGTTATAAAGTAGACGGCGAAACATTTAAGG | |
| | | CCATTAAAAATTACAGAAAGCAAAGATTCAAATGGCTAACCGAATA | |
| | | CACAGGAGAGAATCACATAAACAGCACATTTGCAAAGGGCAAATAT | |
| | | GATGAATACGAGTACCACCTAGGGTGGTACTCTAACATATTTATAG | |
| | | GCAACCTTAGACACAACCTGGCATTCCGCTCAGCATACATAGATGT | |
| | | AACTTACAACCCCACAGTAGACAAAGGCAAAGGCAACATAGTGTG | |
| | | GTTCCAGTACCTGACAAAACCCACCACACAGCTGATAAGAACACA | |
| | | GGCAAAATGCGTTATAGAAGACCTGCCACTTTACTGTGCCTTTTTT | |
| | | GGCTACGAGGACTATATACAGAGAACACTAGGCCCTTACCAGGAC | |
| | | ATAGAGACAGTAGGCGTCATCTGCTTTATAAGCCCCTACACAGAAC | |
| | | CTCCATGTATTAGAAAAGAAGAGCAAAAAAAGGACTGGGGCTTTGT | |
| | | ATTTTATGACACCAACTTTGGAAACGGAAAAACACCAGAGGGCATA | |
| | | GGCCAAGTTCACCCCTACTGGATGCAGAGGTGGAGAGTAATGGCC | |
| | | CAGTTTCAAAAAGAAACTCAAAACAGAATTGCCAGGAGCGGACCG | |
| | | TTTAGCTACAGAGACGACATACCCTCAGCCACACTGACTGCCAACT | |
| | | ACAAGTTCTACTTTAACTGGGGGGGCGACTCTATATTTCCACAGAT | |
| | | TATTAAGAACCCCTGCCCCGACACCGGGCTGCGACCCAGTGGCC | |
| | | ATAGAGAGCCTCGCTCAGTACAAGTCGTTAGCCCGCTCACCATGG | |
| | | GACCAGAGTTCATATTCCACCGCTGGGACTGGCGACGGGGGTTCT | |
| | | ATAATCCAAAAGCTCTCAAACGAATGCTTGAAAAATCAGATAATGAT | |
| | | GCAGAGTCTTCAACAGGCCCAAAAGTGCCTCGGTGGTTTCCAGCA | |
| | | CACCACGACCAAGAGCAAGAAAGCGACTTCGATTCACAAGAGACA | |
| | | AGGTCGCAGTCCTCGCAAGAAGAAGCCGCTCAAGAAGCCCTCCAA | |
| | | GACGTCCAAGAGACGTCGGTACAGCAGTACCTCCTCAAGCAGTTC | |
| | | CGAGAGCAGCGGCTACTCGGACAGCAACTCCGCCTCCTCATGCTC | |
| | | CAACTCACCAAGACGCAAAGCAATCTCCACATAAATCCCCGTGTCC | |
| | | TTGACCATGCATAA | |
| ABY26046.1 | EU305676.1 | ATGTTCTGGTGGGGATGGCGCCGCCGATGGTGGTGGAAGCCACG | 211 |
| | | GAGGCGATGGAGACGCAGGAGGGCGCGCCGCCCGAGACGAGTA | |
| | | CCGCGAAGACGATATAGAAGAGCTGCTCGCCGCTATCGAGGCAG | |
| | | ACGAGTAAGGAGGCGCCGCGCGGGGGGCTGGCGGGGGCGACGT | |
| | | AGATACTCCCGACACTATAGCAGACGACTGACTGTCAGGCGAAAG | |
| | | AAAAAGAAACTGACTCTTAAGATCTGGCAGCCACAGAATATCAGGA | |
| | | AATGTAGAATAAGGGGTCTCCTGCCCCTCCTGATATGCGGGCACA | |
| | | CCCGTTCGGCCTTTAACTATGCCATCCACTCGGATGACAAGACCC | |
| | | CCCAACAGGAGAGTTTCGGGGGCGGCCTCAGCACCGTCAGCTTC | |
| | | TCCTTAAAAGTACTGTTTGACCAGAACCAGAGGGGACTTAATAGGT | |
| | | GGTCGGCCAGCAACGACCAACTGGACCTTGCTCGGTACCTGGGG | |
| | | TGCACTTTCTGGTTCTACAGAGACAAAAAGACTGATTTTATAGTGC | |
| | | AGTATGATATCAGCGCCCCCTTCAAGCTGGACAAAAACAGCAGTC | |
| | | CCAGCTACCACCCCTTCATGCTCATGAAGGCAAAACACAAGGTGC | |
| | | TAATTCCCAGCTTTGACACTAAACCCAAGGGCAGGGAAAAAATTAA | |
| | | AGTTAGAATACAGCCCCCCAAAATGTTCATAGACAAGTGGTACACA | |
| | | CAAGAGGACCTGTGTCCCGTTATTCTTGTGTCACTTGCGGTTAGC | |
| | | GTAGCTTCCTTTACACATCCGTTCTGCTCACCACAAACTGCCAATC | |
| | | CTTGCATCACCTTCCAGGTTTTGAAAGAGTTCTATTACCCAGCCAT | |
| | | GGGCTATGGGGCCCCTGAAACAACTGTCACTTCTGTATTTAACACT | |
| | | TTATATACCACAGCCACCTACTGGCAGTCTCACCTTACCCCCCAGT | |
| | | TTGTCAGAATGCCCACCAAAAACCCAGACAATACTGAAAACAACCA | |
| | | AGCTCAAGCCTTTAATACCTGGGTTGATAAAGATTTCAAAACAGGC | |
| | | AAGTTAGTAAAGTATAACTTTCCCCAGTATGCTCCTTCAATAGAGAA | |
| | | ACTAAAACAATTAAGAACATACTACTTTGAATGGGAAACTAAACACA | |
| | | CTGGGGTTGCAGCACCACCTACCTGGACCACCCCTACCTCAGACA | |
| | | GATACGAGTACCATATGGGAATGTTCAGTCCCACTTTCCTCACACC | |
| | | GTTCAGGTCAGCTGGCCTAGACTTTCCCGGAGCCTACCAGGACGT | |
| | | CACCTACAATCCCCTCACAGACAAGGGGGTGGGCAACAGAATGTG | |
| | | GTTCCAATACAACACCAAGATAGACACTCAGTTCGACGCCAGGTC | |
| | | CTGCAAGTGCGTACTAGAGGACATGCCCCTGTACGCCATGGCCTA | |
| | | CGGGTATGCAGACTTTTTAGAGCAAGAGATAGGAGAGTACCAGGA | |
| | | CCTAGAGGCCAACGGGTACGTCTGTGTAATAAGCCCCTACACCAA | |
| | | ACCCCCAATGTTCAACAAACACAACCCGCAACAGGGGTACGTATT | |
| | | CTATGACTCTCAGTGGGGCAACGGCAAGTGGATAGACGGAACCG | |
| | | GGTTCGTGCCCGTCTACTGGCTGACCAGATGGAGAGTAGAGCTGC | |
| | | TATTTCAGAAAAAAGTACTGTCAGACATCGCCATGTCAGGCCCCTT | |
| | | CAGCTACCCAGACGAACTTAAAAACACTGTACTGACGGCCAAATAC | |
| | | AGATTTGACTTTAAGTGGGGTGGCAATCTCTTCCACCAGCAGACCA | |
| | | TTAGAAACCCCTGCAAACCAGAAGAGACCTCGACCGGTAGAGTCC | |
| | | CTCGCGATGTACAAGTCGTTGACCCGGTCACCATGGGCCCCAGAT | |
| | | TCGTCTTTCACTCCTGGGACTGGAGGCGAGGGTTCCTTAGTGACA | |
| | | GAGCTCTCAAAAGAATGTTTGAAAAACCGCTCGATCTTGAGGGATT | |
| | | TGCAGCGTCTCCAAAACGACCTCGCATATTCCCTCCCACAGAGGG | |
| | | ACAGCTCGCCCGAGAGCAAAAAGAGCAAGAAGAAAGCTCAGATTC | |
| | | GCAGGAAGAAAGCAGCCTTACCTCGCTCGAAGAAGTCCCGGAAGA | |
| | | GACGAAGCTACGACTCCACCTCAGAAAGCAGCTCAGAGAGCAGC | |
| | | GAAGCATCAGACAGCAACTCCGAACCATGTTCCAGCAACTTGTCA | |
| | | AGACGCAAGCGGGCCTACACCTAAACCCCCTTTTATCTTCCCAGC | |
| | | TGTAA | |
| ACK44071.1 | FJ426280.1 | ATGGCCTGGCGATGGTGGTGGCAGAGACGATGGCGCCGCCGCCC | 212 |
| | | GTGGCCCCGCAGACGGTGGAGACGCCTACGACGCCGGAGACCTC | |
| | | GACGACCTGTTCGCCGCCGTCGAAGACGAGCAACAGTAAGGAGG | |
| | | CGGAGGTGGAGGGGCAGACGTGGGCGACGCACATACACCCGAC | |
| | | GCGCGGTCAGACGCAGACGCAGACCCAGAAAGAGATTTGTACTGA | |
| | | CTCAGTGGAGCCCCCAGACAGCCAGAAACTGTTCAATAAGGGGCA | |
| | | TAGTGCCCATGGTAATATGCGGACACACCAGAGCAGGTAGAAACT | |
| | | ATGCCCTTCACAGCGAGGACTTTACCACTCAGATAAGACCCTTTGG | |
| | | AGGCAGCTTCAGCACAACCACCTGGTCCCTAAAAGTACTGTGGGA | |
| | | CGAACACCAGAAATTCCAAAACAGATGGTCCTACCCAAACACACA | |
| | | GCTGGACCTAGCCAGGTACAGGGGGGTCACCTTCTGGTTCTACAG | |
| | | AGACCAGAAAACAGACTATATAGTACAATGGAGCAGAAATCCTCCC | |
| | | TTTAAACTAAACAAATACAGCAGCCCCATGTACCACCCTGGAATGA | |
| | | TGATGCAGGCAAAAAAGAAACTGGTGGTCCCCAGTTTCCAGACCA | |
| | | GACCTAAAGGCAAAAAGAGATACAGAGTCAGAATAAGACCCCCCA | |
| | | ACATGTTCAATGACAAGTGGTACACTCAAGAGGACCTTTGTCCAGT | |
| | | ACCTCTTGTGCAAATTGTGGTTTCTGCGGCTACCCAGACAAAAAAG | |
| | | AACTGCTCACCACAAACGAACAACCCTTGCATCACTTTCCAGGTTT | |
| | | TGAAAGACAAGTACTTAAACTACATAGGAGTTAACTCTTCCGAGAC | |
| | | CCGAAGAAACAGTTATAAAACTCTACAAGAGAAACTTTACTCACAA | |
| | | TGCACATACTTTCAAACCACACAAGTTTTAGCTCAATTATCTCCAGC | |
| | | ATTTCAGCCCGCAAAGAAACCTAACAGAACCAACAACTCAACCAG | |
| | | CACAACACTAGGCAACAAAGTCACAGACCTAAAATCCAACAATGG | |
| | | CAAATTCCACACAGGCAACAACCCAGTGTTTGGCATGTGTTCATAT | |
| | | AAACCCAGCAAGGACATACTATATAAAGCAAACGAATGGTTGTGG | |
| | | GACAATCTCATGGTTGAAAATGATTTACATTCCACATATGGCAAGG | |
| | | CAACCCTTAAATGCATGGAGTACCACACAGGCATTTACAGCTCCAT | |
| | | ATTCCTAAGTCCTCAAAGGTCCCTAGAATTCCCAGCAGCATACCAA | |
| | | GATGTCACATACAACCCAAACTGTGACAGAGCCATAGGCAACCGT | |
| | | GTATGGTTCCAATATGGCACAAAAATGAACACAAACTTTAATGAAC | |
| | | AACAGTGTAAGTGTGTGTTAACAAACATTCCCCTGTGGGCGGCCTT | |
| | | TAACGGCTACCCAGACTTTATAGAACAAGAACTCGGTATCAGCACA | |
| | | GAGGTACACAACTTTGGCATAGTATGTTTCCAGTGCCCCTACACCT | |
| | | TTCCCCCACTCTATGACAAAAAGAACCCAGATAAAGGCTACGTATT | |
| | | TTATGACACCACCTTTGGGAACGGAAAAATGCCAGACGGGTCAGG | |
| | | CCACATTCCCATCTACTGGCAGCAGAGATGGTGGATCAGACTAGC | |
| | | CTTTCAAGTACAAGTCATGCATGACTTTGTACTCACTGGCCCCTTT | |
| | | AGCTACAAAGATGACCTAGCAAACACTACACTAACAGCCAGGTAC | |
| | | AAGTTCAGATTCAAATGGGGCGGTAATATCATCCCCGAACAGATTA | |
| | | TCAAGAACCCGTGTAAGAGAGAACAGTCCCTCGGTTCCTACCCCG | |
| | | ATAGACAACGTCGCGACCTACAAGTTGTTGACCCATCAACCATGG | |
| | | GCCCGATCTACACCTTCCACACATGGGACTGGCGACGGGGGCTTT | |
| | | TTGGTGCAGATGCTATCCAGAGAGTGTCACAAAAACCGGAAGATG | |
| | | CTCTCCGCTTTACAAACCCTTTCAAGAGACCCAGATATCTTCCCCC | |
| | | GACAGACGGAGAAGACTACCGACAAGAAGAAGACTTCGCTTTACA | |
| | | GGAAAGAAGACGGCGCACATCCACAGAAGAAGTCCAGGACGAGG | |
| | | AGAGCCCCCCGCAAAACGCGCCGCTCCTACAGCAGCAGCAGCAG | |
| | | CAGCGGGAGCTCTCAGTCCAGCACGCGGAGCAGCAGCGACTCGG | |
| | | AGTCCAACTCCGATACATCCTCCAAGAAGTCCTCAAAACGCAAGC | |
| | | GGGTCTCCACCTAAACCCCCTATTATTAGGCCCGCCACAAACAAG | |
| | | GTGTATATCTTTGAGCCCCCCAGAGGCCTACTCCCCATAG | |
| ACR20257.1 | FJ392105.1 | ATGGCAGCCTGGTGGTGGGGCAGGCGGAGACGCTGGCGCAGGT | 213 |
| | | GGAGGCGCCGCCGCCTCCCTCGCCGCCGCCGCTGGCGACGGAG | |
| | | GAGACGGTGGCCCAGAAGACGCAGGCGGAGATGGCCGCGCAGA | |
| | | CGCAGACGTCGCAGACCTGCTCGCCGCCCTAGAAGGAGACGCAG | |
| | | ACGCCGAAGGGTAAGGAGACCTCGCCGGCGCCAAAAACTGGTAC | |
| | | TGACTCAGTGGAACCCTCAGACAGTTAGAAAGTGCATTATCAGAG | |
| | | GGTTCGTGCCGCTGTTCCAGTGCAGCAGAACTGCCTACCACAGGA | |
| | | ACTTTGTAGACCACATGGACGACGTGTACACCACGGGTCCCTTCG | |
| | | GGGGCGGCACGGGGTCCATGCTTTTCACCCTGAGCTTCTTCTACC | |
| | | ACGAGTTTAAAAAGCACCACTGCAAGTGGTCCGCCAGCAACAGAG | |
| | | ACTTTGACTTGTGTAGATACAGGGGCACGGTTCTAAAGTTTTATAG | |
| | | ACATCCAGACGTAGACTACATAGTTTGGCTGAACAGAAACCCCCCT | |
| | | TTCCAGGAAAACCTATTAGACGCCATGAGCAGACAGCCCCTCATA | |
| | | ATGTTACAGACTCACAAGTGCATACTGGTGAGGAGCTTTAAAACGC | |
| | | ACCCCAGGGGACCCTCGTACGTCAGAATGAAAGTTAGACCCCCGA | |
| | | GACTACTTACAGACAAGTGGTACTTTCAGTCAGACTTCTGCAACGT | |
| | | TCCGCTTTTCCAGCTACAGTTTGCTCTTGCGGAACTGCGGTTTCCG | |
| | | ATCGGCTCACCACAAACGAACACCACTTGTGTAAACTTCCTGGTGT | |
| | | TAGATAACAGGTACCACTTATTTTTAGATAACAAACCACAACAGTCA | |
| | | GACAACTCACAAAGAGAAGAGAGGGGGCACGGTTATCCCTTTAAC | |
| | | GGTAGTGAGGGAGAAGCTGATAGACTAAAATTCTGGCACAGTTTG | |
| | | TGGAATACAGGCAGATTCCTAAACACCACTCACATTAACACCCTAC | |
| | | AGCCAAACATCTCTAAATTACAAGAACATAAAGCTGAAGACACAGA | |
| | | GGCAAAAACTACCTATAAAAGTTTAATTAACGGTAACAAAAAGGTA | |
| | | TATAACGATAGTCAATACATGCAAAACGTTTGGGCACAAAACAAAA | |
| | | TAAATACCCTTTATGAGGCTATAGCAGAAGAACAATACAGAAAAAT | |
| | | ACAAAAGTACTATAACACCACATACGGGCAGTACCAAAGGCAACTA | |
| | | TTTACAGGCAAGAAGTACTGGGACTACAGAGTAGGCATGTTCAGT | |
| | | CCCACCTTCCTAAGTCCCAGCAGACTAAATCCAGAGATGCCAGGT | |
| | | GCCTACACAGAGATAGCCTATAACCCCTGGACAGACGAGGGCACG | |
| | | GGCAACGTTGTGTGCCTGCAGTACCTAACAAAAGAAACCTCAGAC | |
| | | TACAAGCCACACGCAGGTAGCAAATTCACCATAGAGGACGTACCC | |
| | | CTGTGGATAGCCATGAATGGGTACGTGGACATATGTAAAAAAGAG | |
| | | GGCAAAGATCCAGGCATAAGACTAAACTGCCTTATGTGTATAAGGT | |
| | | GCCCGTACACCAGGCCCAAACTTTACAACCCCAGATACCCCAAAG | |
| | | AACTGTTTGTAGTGTACTCTTACAACTTTGCCCACGGGCGCATGCC | |
| | | CGGGGGGGACAAATACATACCCATGGAGTTTAAGGACAGGTGGTA | |
| | | CCCGTCGCTCATGCACCAGGAAGAGGTCATAGAGGACATAGTCAG | |
| | | GAGCGGCCCCTTTGCCCTAAAAGACCAGACAGAGATGGTTACTTG | |
| | | CATGATGAGGTACTCGGCCCTGTTTAACTGGGGCGGTAATATTATC | |
| | | CGCGAACAGGCCGTGGAAGACCCCTGTAAAAAGAACACCTTTGCC | |
| | | CTTCCCGGAGCCAGTGGAGTCGCTCGCCTACTACAAGTCAGCAAC | |
| | | CCGATCAGGCAGACCCCCAGCACCACCTGGCACTCGTGGGACTG | |
| | | GAGAAGGTCCCTCTTTACACAAACGGGTATTAAAAGAATGCGCGA | |
| | | ACAACAACCGTATGATGAAATTACTTATGCAGGGCCTAAGAGGCCA | |
| | | AAACTCACAGTTCCCGCAGGACCCACCCTCGCTGCCGGAGACGC | |
| | | CTACAACTACTGGGAAAGAAAACCGCTCACCTCGCCCGGAGAGAC | |
| | | GCTCCCGACCCAGACGGAGACAGAGACAGAAGCCCCAGAGGAAG | |
| | | AAGCCCAGCAAGAAGAAGTCCAGGAGGGCCTCCAGCTCCAGCAG | |
| | | CTCTGGGAGCAGCAACTCCAGCAAAAGCGACAGCTGGGAGTCAT | |
| | | GTTCCAGCAACTCCTCCGACTCAGAACGGGGGCGGAAATACACCC | |
| | | GGCCCTCGCATAG | |
| ACR20260.1 | FJ392107.1 | ATGGCAGCCTGGTGGTGGGGCAGGCGGAGACGCTGGCGCAGGT | 214 |
| | | GGAGGCGCCGCCGCCTCCCTCGCCGCCGCCGCTGGCGACGGAG | |
| | | GAGACGGTGGCCCAGAAGACGCAGGCGGAGATGGCCGCGCAGA | |
| | | CGCAGACGTCGCAGACCTGCTCGCCGCCCTAGAAGGAGACGCAG | |
| | | ACGCCGAAGGGTAAGGAGACCTCGCCGGCGCCAAAAACTGGTAC | |
| | | TGACTCAGTGGAACCCTCAGACAGTTAGAAAGTGCATTATCAGAG | |
| | | GGTTCGTGCCGCTGTTCCAGTGCAGCAGAACTGCCTGCCACAGGA | |
| | | ACTTTGTAGACCACATGGACGACGTGTACACCACGGGTCCCTTCG | |
| | | GGGGCGGCACGGGGTCCATGCTTTTCACCCTGAGCTTCTTCTACC | |
| | | ACGAGTTTAAAAAGCACCACTGCAAGTGGTCCGCCAGCAACAGAG | |
| | | ACTTTGACTTGTGTAGATACAGGGGCACGGTTCTAAAGTTTTATAG | |
| | | ACATCCAGACGTAGACTACATAGTTTGGCTGAACAGAAACCCCCCT | |
| | | TTCCAGGAAAACCTATTAGACGCCATGAGCAGACAGCCCCTCATA | |
| | | ATGTTACAGACTCACAAGTGCATACTGGTGAGGAGCTTTAAAACGC | |
| | | ACCCCAGGGGACCCTCGTACGTCAGAATGAAAGTTAGACCCCCGA | |
| | | GACTACTTACAGACAAGTGGTACTTTCAGTCAGACTTCTGCAACGT | |
| | | TCCGCTTTTCCAGCTACAGTTTGCTCTTGCGGAACTGCGGTTTCCG | |
| | | ATCGGCTCACCACAAACGAACACCACTTGTGTAAACTTCCTGGTGT | |
| | | TAGATAACAGGTACCACTTATTTTTAGATAACAAACCACAACAGTCA | |
| | | GAGAACCTACAAAGAAAAGAGAGGGGGCACGGTTATTCCTTTACG | |
| | | GGTAATGAGGGAGAAGTTGATAGACTAAAATTCTGGCACAGTTTGT | |
| | | GGAATACAGGCAGATTCCTAAACACCACTCACATTAACACCCTACT | |
| | | GCCAAACATCTCTAAATTACAAGAACATAAAGCTGAAGACAGACAG | |
| | | GCAAATGCTAAGTATAAAAATTTAATTAACGGTAACAAAAAGGTATA | |
| | | TAACGATAGTCAATACATGCAAAACGTTTGGGAAGAAAACAAAATA | |
| | | AATACCCTTTATGACGCTATAGCAGAAGAACAATACAGAAAAATAC | |
| | | AAAAGTACTATAACACCACATACGGGCAGTACCAAAGGCAACTATT | |
| | | TACAGGCAAGAAGTACTGGGACTACAGAGTAGGCATGTTCAGTCC | |
| | | CACCTTCCTAAGTCCCAGCAGACTAAATCCAGAGATGCCAGGTGC | |
| | | CTACACAGAGATAGCCTATAACCCCTGGACAGACGAGGGCACGG | |
| | | GCAACGTTGTGTGCCTGCAGTACCTAACAAAAGAAACCTCAGACTA | |
| | | CAAGCCACACGCAGGTAGCAAATTCACCATAGAGGACGTACCCCT | |
| | | GTGGATAGCCATGAACGGGTACGTGGACATATGTAAAAAAGAGGG | |
| | | CAAAGATCCAGGCATAAGACTAAACTGCCTTATGTGTATAAGGTGT | |
| | | CCGTACACCAGGCCCAAACTTTACAACCCCAGATACCCCGAAGAA | |
| | | CTGTTTGTAGTGTACTCTTACAACTTTGCCCACGGGCGCATGCCC | |
| | | GGGGGGGACAAATACATACCCATGGAGTTTAAGGACAGGTGGTAC | |
| | | CCGTCGCTCATGCACCAGGAAGAGGTCATAGAGGACATAGTCAGG | |
| | | AGCGGCCCCTTTGCCCTAAAAGACCAGACAGAGATGGTTACTTGC | |
| | | ATGATGAGGTACTCGGCCCTGTTTAACTGGGGCGGTAATATTATCC | |
| | | GCGAACAGGCCGTGGAAGACCCCTGTAAAAAGAACACCTTTGCCC | |
| | | TTCCCGGAGCCAGTGGAGTCGCTCGCCTACTACAAGTCAGCAACC | |
| | | CGATCAGGCAGACCCCCAGCACCACCTGGCACTCGTGGGACTGG | |
| | | AGAAGGTCCCTCTTTACACAAACGGGTATTAAAAGAATGCGCGAAC | |
| | | AACAACCGTATGATGAAATTACTTATGCAGGGCCTAAGAGGCCAAA | |
| | | ACTCACAGTTCCCGCAGGGCCCACCCTCGCTGCCGGAGACGCCT | |
| | | ACAACTACTGGGAAAGAAAACCGCTCACCTCGCCCGGAGAGACGC | |
| | | TCCCGACCCAGACGGATACAGAGACAGAAGCCCCAGAGGAAGAA | |
| | | GCCCAGCAAGAAGAAGTCCAGGAGGGCCTCCAGCTCCAGCAGCT | |
| | | CTGGGAGCAGCAACTCCAGCAAAAGCGACAGCTGGGAGTCATGTT | |
| | | CCAGCAACTCCTCCGACTCAGAACGGGGGCGGAAATACACCCGG | |
| | | CCCTCGCATAG | |
| ACR20262.1 | FJ392108.1 | ATGGCAGCCTGGTGGTGGGGCAGGCGGAGACGCTGGCGCAGGT | 215 |
| | | GGAGGCGCCGCCGCCTCCCTCGCCGCCGCCGCTGGCGACGGAG | |
| | | GAGACGGTGGCCCAGAAGACGCAGGCGGAGATGGCCGCGCAGA | |
| | | CGCAGACGTCGCAGACCTGCTCGCCGCCCTAGAAGGAGACGCAG | |
| | | ACGCCGAAGGGTAAGGAGACCTCGCCGGCGCCAAAAACTGGTAC | |
| | | TGACTCAGTGGAACCCTCAGACAGTTAGAAAGTGCATTATCAGAG | |
| | | GGTTCGTGCCGCTGTTCCAGTGCAGCAGAACTGCCTACCACAGGA | |
| | | ACTTTGTAGACCACATGGACGACGTGTACACCACGGGTCCCTTCG | |
| | | GGGGCGGCACGGGGTCCATGCTTTTCACCCTGAGCTTCTTCTACC | |
| | | ACGAGTTTAAAAAGCACCACTGCAAGTGGTCCGCCAGCAACAGAG | |
| | | ACTTTGACTTGTGTAGATACAGGGGCACGGTTCTAAAGTTTTATAG | |
| | | ACATCCAGACGTAGACTACATAGTTTGGCTGAACAGAAACCCCCCT | |
| | | TTCCAGGAAGACCTATTAGACGCCATGAGCAGACAGCCCCTCATA | |
| | | ATGTTACAGACTCACAAGTGCATACTGGTGAGGAGCTTTAAAACGC | |
| | | ACCCCAGGGGACCCTCGTACGTCAGAATGAAAGTTAGACCCCCGA | |
| | | GACTACTTACAGACAAGTGGTACTTTCAGTCGGACTTCTGCAACGT | |
| | | TCCGCTTTTCCAGCTACAGTTTGCTCTTGCGGAACTGCGGTTTCCG | |
| | | ATCGGCTCACCACAAACGAACACCACTTGTGTAAACTTCCTGGTGT | |
| | | TAGATAACAGGTACCACTTATTTTTAGATAACAAACCACAACAGTCA | |
| | | GACAACCCACAAAGAAAAGAGAGGGGGCACGGTTATTCCTTTACG | |
| | | GGTAATGAGGGAGAAATGGATAGAGAAAGATTCTGGCACAGTTTG | |
| | | TGGAGTACAGGCAGATTCCTAAACACCACTCACATTAACACCCTAC | |
| | | TGCCAAACATCTCTAAATTACAAGACCATAAAGCTGAAGACAAAGA | |
| | | CGCAAAAACTACCTATAAAAGTTTAATTAACGATAACAAAAAGGTAT | |
| | | ATAACGATAGTCAATACATGCAAAACGTTTGGGACCAAAACAAAAT | |
| | | ACATACCCTTTATATGGCTATAGCAGAAGAACAATACAGAAAAATA | |
| | | CAAAAGTACTATAACACCACATACGGGCAGTACCAAAGGCAACTAT | |
| | | TTACAGGCAAGAAGTACTGGGACTACAGAGTAGGCATGTTCAGTC | |
| | | CCACCTTCCTAAGTCCCAGCAGACTAAATCCAGAGATGCCAGGTG | |
| | | CCTACACAGAGATAGCCTATAACCCCTGGACAGACGAGGGCACGG | |
| | | GCAACGTTGTGTGCCTGCAGTACCTAACAAAAGAAACCTCAGACTA | |
| | | CAAGCCACACGCAGGTAGCAAATTCACCATAGAGGACGTACCCCT | |
| | | GTGGATAGCCATGAACGGGTACGTGGACATATGTAAAAAAGAGGG | |
| | | CAAAGATCCAGGCATAAGACTAAACTGCCTTATGTGTATAAGGTGT | |
| | | CCGTACACCAGGCCCAAACTTTACAACCCCAGATACCCCGAAGAA | |
| | | CTGTTTGTAGTGTACTCTTACAACTTTGCCCACGGGCGCATGCCC | |
| | | GGGGGGGACAAATACATACCCATGGAGTTTAAGGACAGGTGGTAC | |
| | | CCGTCGCTCATGCACCAGGAAGAGGTCATAGAGGACATAGTCAGG | |
| | | AGCAGCCCCTTTGCCCTAAAAGACCAGACAGAGATGGTTACTTGC | |
| | | ATGATGAGGTACTCGGCCCTGTTTAACTGGGGCGGTAATATTATCC | |
| | | GCGAACAGGCCGTGGAAGACCCCTGTAAAAAGAACACCTTTGCCC | |
| | | TTCCCGGAGCCAGTGGAGTCGCTCGCCTACTACAAGTCAGCAACC | |
| | | CGATCAGGCAGACCCCCAGCACCACCTGGCACTCGTGGGACTGG | |
| | | AGAAGGTCCCTCTTTACACAAACGGGTATTAAAAGAATGCGCGAAC | |
| | | AACAACCGTATGATGAAATTACTTATGCAGGGCCTAAGAGGCCAAA | |
| | | ACTCACAGTTCCCGCAGGGCCCACCCTCGCTGCCGGAGACGCCT | |
| | | ACAACTACTGGGAAAGAAAACCGCTCACCTCGCCCGGAGAGACGC | |
| | | TCCCGACCCAGACGGAGACAGAGACAGAAGCCCCAGAGGAAGAA | |
| | | GCCCAGCAAGAAGAAGTCCAGGAGGGCCTCCAGCTCCAGCAGCT | |
| | | CTGGGAGCAGCAACTCCAGCAAAAGCGACAGCTGGGAGTCATGTT | |
| | | CCAGCAACTCCTCCGGCTCAGAACGGGGGCGGAAATACACCCGG | |
| | | CCCTCGCATAG | |
| ACR20267.1 | FJ392111.1 | ATGGCAGCCTGGTGGTGGGGCAGGCGGAGACGCTGGCGCAGGT | 216 |
| | | GGAGGCGCCGCCGCCTCCCTCGCCGCCGCCGCTGGCGACGGAG | |
| | | GAGACGGTGGCCCAGAAGACGCAGGCGGAGATGGCCGCGCAGA | |
| | | CGCAGACGTCGCAGACCTGCTCGCCGCCCTAGAAGGAGACGCAG | |
| | | ACGCCGAAGGGTAAGGAGACCTCGCCGGCGCCAAAAACTGGTAC | |
| | | TGACTCAGTGGAACCCTCAGACAGTTAGAAAGTGCATTATCAGAG | |
| | | GGTTCGTGCCGCTGTTCCAGTGCAGCAGAACTGCCTACCACAGGA | |
| | | ACTTTGTAGACCACATGGACGACGTGTACACCACGGGTCCCTTCG | |
| | | GGGGCGGCGCGGGGTCCATGCTTTTCACCCTGAGCTTCTTCTACC | |
| | | ACGAGTTTAAAAAGCACCACTGCAAGTGGTCCGCCAGCAACAGAG | |
| | | ACTTTGACTTGAGTAGATACAGGGGCGCGGTTCTAAAGTTCTATAG | |
| | | ACATCCAGACGTAGACTACATAGTTTGGCTGAACAGAAACCCCCCT | |
| | | TTCCAGGAAAACCTATTAGACGCCATGAGCAGACAGCCCCTCATA | |
| | | ATGTTACAGACTCACAAGTGCATACTGGTGAGGAGCTTTAAAACGC | |
| | | ACCCCAGGGGACCCTCGTACGTCAGAATGAAAGTTAGACCCCCGA | |
| | | GACTACTTACAGACAAGTGGTACTTTCAGTCAGACTTCTGCAACGT | |
| | | TCCGCTTTTCCAGCTACAGTTTGCTCTTGCGGAACTGCGGTTTCCG | |
| | | ATCGGCTCACCACAAACGAACACCACTTGTGTAAACTTCCTGGTGT | |
| | | TAGACAACAGGTACCACTCATTTTTAGATAACAAACCACAACAGTC | |
| | | AGAGAACTCACAAAGAAAAGAGAGGGGGCACGGTTATTCCTTTAC | |
| | | GGGTAAAGAGGGAGAACAGGATAGACTAACATTCTGGCAGAGTTT | |
| | | GTGGAATACAGGCAGATTCCTAAACACCACTCACATTAACACCCTA | |
| | | CTGCCAAACATCTCTAAATTACAAGACCATAAAGCTGAAGACACAG | |
| | | ACGCAAATCCTGACTATAAAAGTTTAATTAACGGTAACAAAAAGGT | |
| | | ATATAACGATAGTCAATACATGCAAAACGTTTGGCAACAAGGCAAA | |
| | | ATAAATACCCTTTGTAACGCTATAGCACAGGAACAATACAGAAAAA | |
| | | TACAAAAGTACTATAACACCACATACGGGCAGTACCAAAGGCAACT | |
| | | ATTTACAGGCAAGAAATACTGGGACTACAGAGTAGGCACGTTCAG | |
| | | TCCCACCTTCCTAAGTCCCAGCAGACTAAATCCAGAGATGCCAGG | |
| | | TGCCTACACAGAGATAGCCTATAACCCCTGGACAGACGAGGGCAC | |
| | | GGGCAACGTTGTGTGCCTGCAGTACCTAACAAAAGAAACCTCAGA | |
| | | CTACAAGCCACACGCAGGTAGCAAATTCACCATAGAGGACGTACC | |
| | | CCTGTGGATAGCCATGAACGGGTACGTGGACATATGTAAAAAAGA | |
| | | GGGCAAAGATCCAGGCATAAGACTAAACTGCCTTATGTGTATAAG | |
| | | GTGTCCGTACACCAGGCCCAAACTTTACAACCCCAGATACCCCGA | |
| | | AGAACTGTTTGTAGTGTACTCTTACAACTTTAGCCACGGGCGCATG | |
| | | CCCGGGGGGGACAAATACATACCCATGGAGTTTAAGGACAGGTG | |
| | | GTACCCGTCGCTCATGCACCAGGAAGAGGTCATAGAGGACATAGT | |
| | | CAGGAGCGGCCCCTTTGCCCTAAAAGACCAGACAGACATGGTTAC | |
| | | TTGCATGATGAGGTACTCGGCCCTGTTTAACTGGGGCGGTAATATT | |
| | | ATCCGCGAACAGGCCGTGGAAGACCCCTGTAAAAAGAACACCTTT | |
| | | GCCCTTCCCGGAGCCAGTGGAGTCGCTCGCCTACTACAAGTCAGC | |
| | | AACCCGATCAGGCAGACCCCCAGCACCACCTGGCACTCGTGGGA | |
| | | CTGGAGAAGGTCCCTCTTTACACAAACGGGTATTAAAAGAATGCG | |
| | | CGAACAACAACCGTATGATGAAATTACTTATGCAGGGCCTAAGAG | |
| | | GCCAAAACTCACAGTTCCCGCAGGGCCCACCCTCGCTGCCGGAG | |
| | | ACGCCTACAACTACTGGGAAAGAAAACCGCTCACCTCGCCCGGAG | |
| | | AGACGCTCCCGACCCAGACGGAGACAGAGACAGAAGCCCCAGAG | |
| | | GAAGAAGCCCAGCAAGAAGAAGTCCAGGAGGGCCTCCAGCTCCA | |
| | | GCAGCTATGGGAGCAGCAACTCCAGCAAAAGCGACAGCTGGGAG | |
| | | TCATGTTCCAGCAACTCCTCCGACTCAGAACGGGGGCGGAAATAC | |
| | | ACCCGGCCCTCGCATAG | |
| ACR20269.1 | FJ392112.1 | ATGGCAGCCTGGTGGTGGGGCAGGCGGAGACGCTGGCGCAGGT | 217 |
| | | GGAGGCGCCGCCGCCTCCCTCGCCGCCGCCGCTGGCGACGGAG | |
| | | GAGACGGTGGCCCAGAAGACGCAGGCGGAGATGGCCGCGCAGA | |
| | | CGCAGACGTCGCAGACCTGCTCGCCGCCCTAGAAGGAGACGCAG | |
| | | ACGCCGAAGGGTAAGGAGACCTCGCCGGCGCCAAAAACTGGTAC | |
| | | TGACTCAGTGGAACCCTCAGACAGTTAGAAAGTGCATTATCAGAG | |
| | | GGTTCGTGCCGCTGTTCCAGTGCAGCAGAACTGCCTACCACAGGA | |
| | | ACTTTGTAGACCACATGGACGACGTGTACACCACGGGTCCCTTCG | |
| | | GGGGCGGCACGGGGTCCATGCTTTTCACCCTGAGCTTCTTCTACC | |
| | | ACGAGTTTAAAAAGCACCACTGCAAGTGGTCCGCCAGCAACAGAG | |
| | | ACTTTGACTTGTGTAGATACAGGGGCACGGTTCTAAAGTTTTATAG | |
| | | ACATCCAGACGTAGACTACATAGTTTGGCTGAACAGAAACCCCCCT | |
| | | TTCCAGGAAAACCTATTAGACGCCATGAGCAGACAGCCCCTCATA | |
| | | ATGTTACAGACTCACAAGTGCATACTGGTGAGGAGCTTTAAAACGC | |
| | | ACCCCAGGGGACCCTCGTACGTCAGAATGAAAGTTAGACCCCCGA | |
| | | GACTACTTACAGACAAGTGGTACTTTCAGTCAGACTTCTGCAACGT | |
| | | TCCGCTTTTCCAGCTACAGTTTGCTCTTGCGGAACTGCGGTTTCCG | |
| | | ATCGGCTCACCACAAACGAACACCACTTGTGTAAACTTCCTGGTGT | |
| | | TAGATAACAGGTACCACTTATTTTTAGATAACAAACCACGACAGTC | |
| | | AGAGAACTTACAAAGAAAAGAGAGGGGGCACGGTTATGTCTTTAC | |
| | | GGGTAATGAGGGAGAAGATGATAGACTAAAATTCTGGCACAGTTT | |
| | | GTGGAGTACAGGCAGATTCCTAAACACCACTCACATTAACACCCTA | |
| | | CTGCCAAACATCTCTAAATTACAAGACCATGAAGCTGAAGACACAC | |
| | | AGGCAAAAACTGACTATAAAAGTTTAATTAACGGTAACAAAAAGGT | |
| | | ATATAACGATAGTCAATACATGCAAGACGTTTGGGAACAAAAGAAA | |
| | | ATACAAACCCTTTATAAGGTTATAGCAGAAGAACAATACAGAAAAA | |
| | | TAGAAAAGTACTATAACACCACATACGGGCAGTACCAAAGGCAACT | |
| | | ATTTACAGGCAAGAAGTACTGGGACTACAGAGTAGGCATGTTCAG | |
| | | TCCCACCTTCCTAAGTCCCAGCAGACTAAATCCAGAGATGCCAGG | |
| | | TGCCTACACAGAGATAGCCTATAACCCCTGGACAGACGAGGGCAC | |
| | | GGGCAACGTTGTGTGCCTGCAGTACCTAACAAAAGAAACCTCAGA | |
| | | CTACAAGCCACACGCAGGTAGCAAATTCACCATAGAGGACGTACC | |
| | | CCTGTGGATAGCCATGAACGGGTACGTGGACATATGTAAAAAAGA | |
| | | GGGCAAAGATCCAGGCATAAGACTAAACTGCCTTATGTGTATAAG | |
| | | GTGTCCGTACACCAGGCCCAAACTTTACAACCCCAGATACCCCGA | |
| | | AGAACTGTTTGTAGTGTACTCTTACAACTTTGCCCACGGGCGCATG | |
| | | CCCGGGGGGGACAAATACATACCCATGGAGTTTAAGGACAGGTG | |
| | | GTACCCGTCGCTCATGCACCAGGAAGAGGTCATAGAGGACATAGT | |
| | | CAGGAGCGGCCCCTTTGCCCTAAAAGACCAGACAGAGATGGTTAC | |
| | | TTGCATGATGAGGTACTCGGCCCTGTTTAACTGGGGCGGTAATATT | |
| | | ATCCGCGAACAGGCCGTGGAAGACCCCTGTAAAAAGAACACCTTT | |
| | | GCCCTTCCCGGAGCCAGTGGAGTCGCTCGCCTACTACAAGTCAGC | |
| | | AACCCGATCAGGCAGACCCCCAGCACCACCTGGCACTCGTGGGA | |
| | | CTGGAGAAGGTCCCTCTTTACACAAACGGGTATTAAAAGAATGCG | |
| | | CGAACAACAACCGTATGATGAAATTACTTATGCAGGGCCTAAGAG | |
| | | GCCAAAACTCACAGTTCCCGCAGGGCCCACCCTCGCTGCCGGAG | |
| | | ACGCCTACAACTACTGGGAAAGAAAACCGCTCACCTCGCCCGGAG | |
| | | AGACGCTCCCGACCCAGACGGAGACAGAGACAGAAGCCCCAGAG | |
| | | GAAGAAGCCCAGCAAGAAGAAGTCCAGGAGGGCCTCCAGCTCCA | |
| | | GCAGCTCTGGGAGCAGCAACTCCAGCAAAAGCGACAGCTGGGAG | |
| | | TCATGTTCCAGCAACTCCTCCGACTCAGAACGGGGGCGGAAATAC | |
| | | ACCCGGCCCTCGCATAG | |
| ACR20272.1 | FJ392114.1 | ATGGCTGCCTGGTGGTGGGGCAGGAGGCGGCGATGGCGCCGGT | 218 |
| | | GGAGACGGCGCCGTCTCCCTCGCCGCCGCCGCTGGCGACGGAG | |
| | | GAGACGGTGGCCCAGGAGGCGTAGGCGGAGATGGCCGCGGAGA | |
| | | CGCAGACGTCGCGGACCTGCTCGCCGCCTTAGAAGGAGACGTCG | |
| | | ACGCAGAAGGGTAAGGAGACCTCGCCGGCGCCAAAAACTCGTAC | |
| | | TGACTCAGTGGAACCCCCAGACCCAGAGAAAGTGCGTGGTCAGG | |
| | | GGGTTTCTGCCCCTGTTCTTTTGCGGACAGGGAGCCTATCACAGA | |
| | | AACTTTGTGGAACACATGGACGACGTGTTCCCCAAGGGACCCTCG | |
| | | GGAGGGGGCTTTGGCAGCATGGTGTGGAACCTAGATTTTTTGTAC | |
| | | CAAGAGTTTAAAAAGCATCACAACAAGTGGTCTTCCAGCAACAGG | |
| | | GACTTTGACCTAGTGAGGTGCCACGGCACGGTGATTAAATTCTAC | |
| | | AGACACTCTGACTTTGACTACCTGGTGCACGTCACCAGGACCCCT | |
| | | CCTTTCAAGGAGGACCTCCTCACCATCGTCAGCCACCAGCCGGGG | |
| | | CTCATGATGCAGAACTACAGGTGCATACTCGTAAAGAGTTACAAGA | |
| | | CGCACCCCGGGGGGCGACCCTACATAACACCTAAAATAAGGCCC | |
| | | CCCAGACTCCTGACGGACAAGTGGTACTTTCGGCCCGACTTCTGC | |
| | | GGAGTTCCTCTTTTCAAACTGTACGTTACTCTTGCAGAGTTGCGGT | |
| | | TTCCGATCTGCTCACCACAAACTGACACCAATTGTGTCACCTTCCT | |
| | | GGTGTTAGACAACACCTACTACGACTACTTAGACAATACTGCAGAC | |
| | | ACCACTAGAGACCATGAAAGACAGCAGAAATGGACAAACATGAAA | |
| | | ATGACACCCAGATACCATCTCACCAGTCACATAAATACATTGTTTA | |
| | | GTGGAACACAACAGATGCAAAGCGCAAAAGAAACAGGCAAAGACA | |
| | | GTCAGTTTAGAGAAAACATCTGGAAAACAGCTGAGGTTGTTAAAAT | |
| | | TATTAAAGATATAGCCTCAAAAAACATGCAAAAACAACAAACCTACT | |
| | | ACACAAAAACCTATGGCGCCTATGCCACCCAGTATTTTACTGGAAA | |
| | | ACAATACTGGGACTGGAGGGTGGGCCTGTTCAGCCCCATATTCCT | |
| | | CAGTCCCAGCAGACTGAACCCACAAGAGCCAGGGGCCTACACAG | |
| | | AAATAGCTTACAATCCATGGACTGACGAGGGCACGGGCAACATAG | |
| | | TGTGCATTCAGTACCTAACAAAGAAAGACAGTCACTACAAGCCGG | |
| | | GTGCCGGTAGCAAATTCGCAGTGACGGACGTTCCCCTGTGGGCC | |
| | | GCCCTGTTCGGGTACTACGACCAGTGTAAGAAAGAAAGCAAAGAC | |
| | | GCGAACATAAGACTAAACCGCTTGCTGTTAGTCAGGTGCCCTTACA | |
| | | CCAGGCCTAAACTGTACAATCCCAGAGACCCGGACCAACTGTTTG | |
| | | TAATGTACAGCTACAACTTTGGGCACGGACGCATGCCGGGGGGC | |
| | | GACAAGTACGTGCCCATGGAATTTAAGGACAGGTGGTACCCGTGC | |
| | | ATGCTGCACCAAGAAGAAGTAGTGGAGGAGATAGTAAGGTGCGG | |
| | | GCCCTTTGCTCCCAAAGACATGACTCCCTCGGTAACATGCATGGC | |
| | | CAGATACTCATCCCTGTTCACCTGGGGGGGCAATATCATTCGCGA | |
| | | ACAGGCCGTGGAGGACCCCTGTAAAAAATCCACGTTTGCCATTCC | |
| | | CGGAGCCGGTGGACTCGCTCGCATTCTACAAGTCAGCAACCCGCA | |
| | | GAGGCAAGCCCCCACCACCACCTGGCACTCGTGGGGCTGGCGCC | |
| | | GATCCCTCTTTACAGAGACGGGTCTTAAGCGAATGCAGGAACAAC | |
| | | AACCTTACGATGAAATGTCCTATACAGGCCCTAAAAGGCCAAAACT | |
| | | GTCTGTTCCCCCAGCAGCAGAAGGAAACCTCGCTGCAGGAGGAG | |
| | | GCTTATTCTTCAGGGACGGAAAACAGCCTGCCTCGCCAGGAGGCA | |
| | | GTCTCCCGACGCAGTCGGAGACAGAAGCAGAAGCCGAAGACGAA | |
| | | GAAGCCCACCAAGAAGAGACGGAGGAGGGAGCGCAGCTCCAGCA | |
| | | GCTCTGGGAGCAGCAACTCCAACAGAAGCGAGAGCTGGGAATCG | |
| | | TTTTCCAACACCTCCTCCGACTCCGACAGGGGGCGGAAATCCACC | |
| | | CGGGCCTCGTATAA | |
| ACR20274.1 | FJ392115.1 | ATGGCTGCYTGGTGGTGGGGCAGGAGGCGGCGATGGCGCCGGT | 219 |
| | | GGAGACGGCGCCGTYTCCCTCGCCGCCGCCGCTGGCGACGGAG | |
| | | GAGACGGTGGCCCAGGAGGCGTAGGCGGAGATGGCCGCGGAGA | |
| | | CGCAGACGTCGCAGACCTGCTCGCCGCCTTAGAAGGAGACGTCG | |
| | | ACGCAGAAGGGTAAGGAGACCTCGCCGGCGCCAAAAACTCGTAC | |
| | | TGACTCAGTGGAACCCCCAGACCCAGAGAAAGTGCGTGGTCAGG | |
| | | GGGTTTCTGCCCCTGTTCTTCTGCGGACAGGGAGCCTATCACAGA | |
| | | AACTTTGTGGAACACATGGACGACGTGTTCCCCAAGGGACCCTCG | |
| | | GGAGGGGGCTTTGGCAGCATGGTGTGGAACCTAGATTTTTTGTAC | |
| | | CAAGAGTTTAAAAAGCATCACAACAGGTGGTCTTCCAGCAACAGG | |
| | | GACTTTGACCTAGTGAGGTACCACGGCACGGTGATTAAATTCTACA | |
| | | GACACTCTGACTTTGACTACCTGGTGCACGTCACCAGGACCCCTC | |
| | | CTTTCAAGGAGGACCTCCTCACCATCGTCAGCCACCAGCCGGGGC | |
| | | TCATGATGCAGAACTACAGGTGCATACTCGTAAAGAGTTACAAGAC | |
| | | GCACCCCGGGGGGCGACCCTACATAACACTTAAAATAAGGCCCCC | |
| | | CAGACTCCTGACGGACAAGTGGTACTTTCAGCCCGACTTCTGCGG | |
| | | AGTTCCTCTTTTCAAACTGTACGTTACTCTTGCAGAGTTGCGGTTT | |
| | | CCGATCTGCTCACCACAAACTGACACCAATTGTGTCACCTTCCTGG | |
| | | TGTTAGACAACACCTACTACGACTACTTAGACAGTACTGCAGACAC | |
| | | CACTAGAGACAATGAAAGACACCAGAAATGGAAAAACATGATAATG | |
| | | ACACCCAGATACCATCTCACCAGTCACATAAATACATTGTTTAGTG | |
| | | GAACACAACAGATGCAAAACGCAAAAGAAACAGGCAAAGACAGTC | |
| | | AGTTTAGAGAAAACATCTGGAAAACAGAAGAGGTTGTTAAAATTAT | |
| | | TCACGATATAGCCTCTAGAAACATGCAAAAACAAATAACCTACTAC | |
| | | ACAAAAACCTATGGCGCCTATGCCACCCAGTATTTTACTGGAAAAC | |
| | | AATACTGGGACTGGAGGGTGGGCCTGTTCAGCCCCATATTCCTCA | |
| | | GTCCCAGCAGACTGAACCCACAAGAGCCAGGGGCCTACACAGAA | |
| | | ATAGCTTACAATCCATGGACTGACGAGGGCACGGGCAACATAGTG | |
| | | TGCATTCAGTACCTAACAAAGAAAGACAGTCACTACAAGCCGGGT | |
| | | GCCGGTAGCAAATTCGCAGTGACGGACGTTCCCCTGTGGGCCGC | |
| | | CCTGTTCGGGTACTACGACCAGTGTAAGAAAGAAAGCAAAGACGC | |
| | | GAACATAAGACTAAACTGCTTGCTGTTAGTCAGGTGCCCTTACACC | |
| | | AGGCCTAAACTGTACAATCCCAGAGACCCGGACCAACTGTTTGTA | |
| | | ATGTACAGCTACAACTTTGGGCACGGACGCATGCCGGGGGGCGA | |
| | | CAAGTACGTGCCCATGGAATTTAAGGACAGGTGGTACCCGTGCAT | |
| | | GCTGCACCAAGAAGAAGTAGTGGAGGAGATAGTAAGGTGCGGGC | |
| | | CCTTTGCTCCCAAAGACATGACTCCCTCGGTAACATGCATGGCCA | |
| | | GATACTCATCCCTGTTCACCTGGGGGGGCAATATCATTCGCGAAC | |
| | | AGGCCGTGGAGGACCCCTGTAAAAAATCCACGTTTGCCATTCCCG | |
| | | GAGCCGGTGGACTCGCTCGCATTCTACAAGTCAGCAACCCGCAGA | |
| | | GGCAAGCCCCCACGACCACGTGGCACTTGTGGGACTGGCGCCGA | |
| | | TCCCTCTTTACAGAGACGGGTCTTAAGCGAATGCAGGAACAACAA | |
| | | CCTTACGATGAAATGTCTTATACAGGCCCTAAAAGGCCAAAACTGT | |
| | | CCGTTCCCCCAGCAGCAGAAGGAAACCTCGCTGCAGGAGGAGGC | |
| | | TTATTCTTCCGGGACAGAAAACAGCCCACCTCGCCAGGAGGCAGT | |
| | | CTCCCGACGCAGTCGGAGACAGAAGCAGAAGCGGAAGACGAAGA | |
| | | AGCCCACCAAGAAGAGACGGAGGAGGGAGCGCAGCTCCAGCAGC | |
| | | TCTGGGAGCAGCAACTCCAACAGAAGCGAGAGCTGGGAATCGTTT | |
| | | TCCAACACCTCCTCCGACTCCGACAGGGGGCGGAAATCCACCCG | |
| | | GGCCTCGTATAA | |
| ACR20277.1 | FJ392117.1 | ATGGCATGGTGGTGGTGGAGAAGGAGACGCCGCCCGTGGAGAAG | 220 |
| | | GCGCTGGCGCTGGAAGAGACGAGCCCGAGTACGAACCAGGAGAC | |
| | | CTAGACGCGCTGTTCGCCGCCGTCGAAGAAGAGTAAGGAGGCGG | |
| | | AGGAGGGGGTGGAGGAGACTATACAGACGATGGCGACGAAAGGG | |
| | | CAGACGCAGACGCAGACGCAAAAAGTTAGTAATGAAACAGTGGAA | |
| | | CCCCTCCACTGTCAGCAGATGCTATATTGTTGGATACCTGCCTATT | |
| | | ATTATTATGGGACAGGGGACTGCATCCATGAACTATGCATCTCACT | |
| | | CAGACGACGTGTACTACCCCGGACCGTTTGGGGGGGGAATAAGC | |
| | | TCTATGAGGTTTACTTTAAGAATACTGTATGACCAGTTTATGAGAG | |
| | | GACAGAACTTCTGGACTAAGACAAACGAGGACTTGGACCTAGCTA | |
| | | GATTTCTAGGCAGCAAATGGAGGTTCTATAGACACAAAGATGTGGA | |
| | | CTTTATAGTGACTTACGAGACCTCAGCCCCCTTTACAGACTCCCTA | |
| | | GAGTCAGGACCACACCAACACCCAGGCATACAGATGCTAATGAAA | |
| | | AACAAAATACTAATCCCTAGCTTTGCCACCAAACCAAAAGGAAGGT | |
| | | CTAGCATTAAAGTTAGAATACAGCCCCCAAAGCTAATGATAGACAA | |
| | | GTGGTACCCACAAACTGACTTCTGTGAAGTAACGCTGCTAACCATA | |
| | | CATGCAACCGCCTGCAACTTGCGGTTTCCGTTCTGCTCACCACAA | |
| | | ACTGACACTTCCTGTGTTCAGTTTCAAGTGTTGTCATACAACGCTT | |
| | | ACAGGCAGAGAATTTCAATACTTCCTGAATTATGTACTAGAGAAAA | |
| | | GCTTAGGGAGTTTATTAAACAAGTAGTAAAACCAAATTTAACATGCA | |
| | | TAAACACTCTAGCTACTCCATGGTGCTTTAAATTCCCAGAGCTAGA | |
| | | CAAACTACCACCAGTGGCAAACAATGCAACAGGCTGGTCAGTTAA | |
| | | CCCAGATAGCGGAGACGGAGATGTAATATACCAGGAAACTACATT | |
| | | AGAAACCAAATGGATTGCTAACAATGATGTGTGGCATACAAAAGAC | |
| | | CAAAGAGCACACAACAACATACATAGCCAATATGGCATGCCACAAT | |
| | | CAGACGCATTAGAACACAAAACAGGTTACTTCAGTCCAGCATTATT | |
| | | AAGCCCACAAAGACTAAACCCACAGATACCAGGCCTATACATAAAC | |
| | | ATAGTCTACAATCCACTAACAGACAAAGGAGAAGGCAACAAAATTT | |
| | | GGTGTGACCCACTAACAAAAAACACATTTGGCTATGATCCCCCTAA | |
| | | AAGTAAATTCCTTATAGAAAATCTGCCACTGTGGTCTGCAGTAACA | |
| | | GGATACGTAGACTACTGCACGAAAGCCAGCAAAGATGAAAGCTTT | |
| | | AAATACAACTACAGAGTACTTATCCAGACCCCATACACAGTACCAG | |
| | | CACTATACAGTGACTCTGAAACCACCAAAAACAGAGGCTACATTCC | |
| | | CATAGGCACAGACTTTGCATACGGCCGCATGCCTGGGGGAGTACA | |
| | | ACAAATACCAATTAGATGGAGAATGAGGTGGTACCCCATGCTATTT | |
| | | AATCAACAACCAGTACTAGAAGACCTATTCCAGTCAGGCCCCTTTG | |
| | | CATACCAAGGAGATGCTAAATCAGCCACACTAGTCGGCAAATATG | |
| | | CCTTTAAATGGCTATGGGGTGGCAATCGTATCTTCCAACAGGTGGT | |
| | | CAGAGACCCGCGCTCACACCAGCAAGACCAATCAGTTGGTCCCAG | |
| | | TAGACAGCCTAGAGCAGTACAAGTCTTTGACCCGAAGTACCAAGC | |
| | | ACCACAATGGACATTCCACGCGTGGGACATCAGACGTGGTCTGTT | |
| | | TGGCAGACAGGCTATTAAAAGAGTGTCAGCAAAACCAACACCTGA | |
| | | TGAGCTTATATCAACAGGCCCAAAAAGACCTCGGCTGGAAGTCCC | |
| | | CGCGTTCCAAGAAGAGCAAGAAAAAGACTTACTTTTCAGACAGAGA | |
| | | AAACACAAAGCCTGGGAGGACACAACGGAGGAAGAGACAGAAGC | |
| | | CCCCTCAGAAGAGGAGGAAGAGAACCAAGAGCTCCAGCTCGTCA | |
| | | GACGCCTCCAGCAGCAACGAGAGCTGGGACGAGGCCTCAGATGC | |
| | | CTCTTCCAGCAACTAACCCGCACACAGATGGGGCTGCATGTAGAC | |
| | | CCCCAACTATTGGCCCCTGTATAA | |
| AD051761.1 | GU797360.1 | ATGGCATGGGGATGGTGGAAACGAAGGCGCAAGTGGTGGTGGAG | 221 |
| | | ACGACGCTGGACTCGTGGCCGACTTCGCAAACGACGGGCTAGAC | |
| | | GAGCTGGTCGCCGCCCTCGACGAAGAAGAGTAAGGAGACGGAGG | |
| | | GCTTGGAGGCGTGGGCGACGAAAGAGACGGACTTTCAGACGCAG | |
| | | ACGCAGACGAAAGGGTAGGAGACACAGAACCAGACTTATAATAAG | |
| | | ACAATGGCAGCCAGAAATAGTGAGAAAGTGCCTCATAATAGGCTA | |
| | | CTTTCCCATGATTATATGTGGCCAGGGACGCTGGTCAGAGAACTA | |
| | | CAGCAGCCACCTAGAGGACCGTGTAGTAAAACAGGCCTTCGGTGG | |
| | | GGGACACGCGACTACCAGGTGGTCTCTAAAAGTACTGTACGAGGA | |
| | | GAACCTCAGACACTTGAACTTTTGGACCTGGACTAACAGAGACTTA | |
| | | GAACTGGCCAGGTACCTCAAAGTGACGTGGACCTTTTACAGACAC | |
| | | CAAGATGTAGACTTTATAATATACTTTAACAGAAAGAGCCCCATGG | |
| | | GAGGCAACATATACACAGCACCCATGATGCATCCGGGAGCCCTAA | |
| | | TGCTCAGCAAACACAAGATACTAGTAAAAAGCTTTAAAACAAAACC | |
| | | CAAGGGCAAAGCAACAGTTAAAGTGACTATTAAGCCCCCCACTCTA | |
| | | CTAGTAGACAAGTGGTACTTTCAAAAGGACATTTGCGACATGACAC | |
| | | TGTTAAACCTCAATGCCGTTGCGGCTGACTTGCGGTTTCCGTTCTG | |
| | | CTCACCACAAACTGACAACCCTTGCATCAACTTCCAGGTTCTGTCC | |
| | | TCAGTGTATAACAACTTCCTCTCTATAACTGACAATAGACTAACACC | |
| | | AGTCACAGATGATGGCCAGGCTTATTATAAAGCTTTTCTAGACGCT | |
| | | GCATTTACCAAAGACAGAGACTTTAATGCTGTTAATACGTTTAGAA | |
| | | CAATATCTAACTTTTCCCACCCACAACTAGAACTTCCAACTAAAACC | |
| | | ACCAACACATCCCAAGATCAATACTTTAACACTCTAGATGGGTACT | |
| | | GGGGAGACCCCATATATGTACACACACAAAATATAAAACCTGACCA | |
| | | AAACCTTGATAAATGCAAAGAAATACTTACAAACAACATGAAAAACT | |
| | | GGCATAAAAAAGTAAAGTCAGAAAACCCAAGTAGCCTGAACCACA | |
| | | GCTGCTTTGCCCACAATGTAGGCATATTCAGCAGCTCATTCCTATC | |
| | | CGCAGGCAGACTAGCACCAGAAGTTCCAGGCCTGTACACAGATGT | |
| | | TATTTACAACCCATACACAGACAAGGGAAAGGGAAACATGCTATGG | |
| | | GTGGATTACTGTAGCAAAGGAGACAACCTATACAAAGAAGGCCAA | |
| | | AGCAAGTGTCTACTTGCCAACCTACCCCTCTGGATGGCCACAAAC | |
| | | GGTTATATAGACTGGGTAAAAAAAGAAACAGATAACTGGGTTATAA | |
| | | ACACTCAAGCCAGAGTACTCATGGTATGTCCCTACACTTACCCAAA | |
| | | ACTATACCATGAAATACAGCCATTATATGGCTTTGTAGTATACTCAT | |
| | | ATAACTTTGGAGAGGGAAAAATGCCAAACGGGGCCACATACATAC | |
| | | CCTTTAAGTTTAGAAACAAGTGGTATCCAACCATATACATGCAGCA | |
| | | AGCAGTACTAGAAGATATATCCAGATCGGGCCCCTTTGCACTTAAA | |
| | | CAACAGATACCCAGCGCCACACTTACTGCCAAATACAAATTCAAAT | |
| | | TCTTATTTGGCGGTAACCCTACTTCTGAACAGGTTGTTAGAGACCC | |
| | | CTGCACTCAGCCCACCTTCGAACTGCCCGGAGCCAGTACGCAGC | |
| | | CTCCACGAATACAAGTCACGGACCCGAAACTCCTCGGTCCCCACT | |
| | | ACTCATTCCACTCGTGGGACCTCAGACGTGGCTACTATAGCACAA | |
| | | AGAGTATTAAACGAATGTCAGAACACGAAGAACCTTCTGAGTTTAT | |
| | | TTTCCCAGGTCCCAAAAAACCCAGGGTCGACCTCGGGCCAATCCA | |
| | | ACAGCAAGAAAGGCCCTCCGATTCACTCCAAAGAGAATCGAGGCC | |
| | | GTGGGAGACCAGCGAAGAAGAGAGCGAAGCAGAAGTCCAGCAAG | |
| | | AAGAGACGGAGGAGGTGCCCCTCAGACAGCAACTCCTCCACAAC | |
| | | CTCAGAGAGCAGCAGCAACTCCGAAAGGGCCTCCAGTGCGTCTTC | |
| | | CAGCAGCTAATAAAGACGCAGCAGGGGGTTCACATAGACCCATCC | |
| | | CTACTGTAG | |
| AAX94182.1 | D0003341.1 | ATGGCGTGGTCGTGGTGGTGGAGGCGACGGAAACGCTGGTGGCC | 222 |
| | | GCGCAGAAGGAGGCGATGGAGGAGATTTCGCACCCGAAGAGCTA | |
| | | GACGAGCTGTTCCGCGCCGTCGCCGCCGACGAAGAGTAAGGAGG | |
| | | CGCCGGTGGGGGAGGCGAGGACGTAGGAGACGGGTTTTTTATAA | |
| | | GAGACGCAGACGAAAGACTGGCAGACTGTACAGAAAGCCCAAAAA | |
| | | GAAACTAGTACTGACTCAGTGGCACCCCACTACCGTCCGCAACTG | |
| | | CTCCATCCGAGGCCTTGTGCCTCTAGTACTCTGCGGACACACTCA | |
| | | GGGCGGCAGAAACTTTGCTCTCAGGAGCGATGACTACCCCAAGCA | |
| | | GGGGTCTCCTTACGGAGGCAGTTTTAGCACTACAACCTGGAACTT | |
| | | GAGGGTCCTTTTTGACGAACACCAAAAACACCACAACACGTGGAG | |
| | | CTACCCCAATAACCAGCTAGACCTGGGCAGATACAAGGGCTGCAC | |
| | | CTTCTGCTTTTACAGAGGCAAAAAGACGGACTACATAGTAAAGTTT | |
| | | CAGAGGAGGGGACCCTTTAAAATAAACAAGTACAGCAGTCCCATG | |
| | | GCCCATCCGGGCATGATGATGCTAGATAAGATGAAAATCCTGGTG | |
| | | CCCAGCTTTGATACCAGGCCCGGGGGTCGCTGA | |
| AAX94185.1 | DQ003342.1 | ATGGCGTGGTCGTGGTGGTGGAGGCGACGGAAACGCTGGTGGCC | 223 |
| | | GCGCAGAAGGAGGCGATGGAGGAGATTTCGCACCCGAAGAGCTA | |
| | | GACGAGCTGTTCCGCGCCGTCGCCGCCGACGAAGAGTAAGGAGG | |
| | | CGCCGGTGGGGGAGGCGAGGACGTAGGAGACGGGTTTTTTATAA | |
| | | GAGACGCAGACGAAAGACTGGCAGACTGTACAGAAAGCCCAAAAA | |
| | | GAAACTAGTACTGACTCAGTGGCACCCCACTACCGTCCGCAACTG | |
| | | CTCCATCCGAGGCCTTGTGCCTCTAGTACTCTGCGGACACACTCA | |
| | | GGGCGGCAGAAACTTTGCTCTCAGGAGCGATGACTACCCCAAGCA | |
| | | GGGGTCTCCTTACGGAGGCAGTTTTAGCACTACAACCTGGAACTT | |
| | | GAGGGTCCTTTTTGACGAACACCAAAAACACCACAACACGTGGAG | |
| | | CTACCCCAATAACCAGCTAGACCTGGGCAGATACAAGGGCTGCAC | |
| | | CTTCTGCTTTTACAGAGGCAAAAAGACGGACTACATAGTAAAGTTT | |
| | | CAGAGGAGGGGACCCTTTAAAATAAACAAGTACAGCAGTCCCATG | |
| | | GCCCATCCGGGCATGATGATGCTAGATAAGATGAAAATCCTGGTG | |
| | | CCCAGCTTTGATACCAGGCCCGGGGGTCGCTGA | |
| AAX94188.1 | DQ003343.1 | ATGGCGTGGTCGTGGTGGTGGAGGCGACGGAAACGCTGGTGGCC | 224 |
| | | GCGCAGAAGGAGGCGATGGAGGAGATTTCGCACCCGAAGAGCTA | |
| | | GACGAGCTGTTCCGCGCCGTCGCCGCCGACGAAGAGTAAGGAGG | |
| | | CGCCGGTGGGGGAGGCGAAGACGTAGGAGACGGGTTTTTTATAA | |
| | | GAGACGCAGACGAAAGACTGGCAGACTGTACAGAAAGCCCAAAAA | |
| | | GAAACTAGTACTGACTCAGTGGCACCCCACTACCGTCCGCAACTG | |
| | | CTCCATCCGAGGCCTTGTGCCTCTAGTACTCTGCGGACACACTCA | |
| | | GGGCGGCAGAAACTTTGCTCTCAGGAGCGATGACTACCCCAAGCA | |
| | | GGGGTCTCCTTACGGAGGCAGTTTTAGCACTACAACCTGGAACTT | |
| | | GAGGGTCCTTTTTGACGAACACCAAAAACACCACAACACGTGGAG | |
| | | CTACCCCAATAACCAGCTAGACCTGGGCAGATACAAGGGCTGCAC | |
| | | CTTCTACTTTTACAGAGACAAAAAGACAGACTACATAGTAAAGTTTC | |
| | | AGAGGAGGGGACCCTTTAAAATAAACAAGTACAGCAGTCCCATGG | |
| | | CCCATCCGGGCATGATGATGCTAGATAAGATGAAAATCCTGGTGC | |
| | | CCAGCTTTGATACCAGGCCCGGGGGTCGCTGA | |
| AAX94191.1 | DQ003344.1 | ATGGCGTGGTCGTGGTGGTGGAGGCGACGGAAACGCTGGTGGCC | 225 |
| | | GCGCAGAAGGAGGCGATGGAGGAGATTTCGCACCCGAAGAGCTA | |
| | | GACGAGCTGTTCCGCGCCGTCGCCGCCGACGAAGAGTAAGGAGG | |
| | | CGCCGGTGGGGGAGGCGAAGACGTAGGAGACGGGTTTTTTATAA | |
| | | GAGACGCAGACGAAAGACTGGCAGACTGTACAGAAAGCCCAAAAA | |
| | | GAAACTAGTACTGACTCAGTGGCACCCCACTACCGTCCGCAACTG | |
| | | CTCCATCCGAGGCCTTGTGCCTCTAGTACTCTGCGGACACACTCA | |
| | | GGGCGGCAGAAACTTTGCTCTCAGGAGCGATGACTACCCCAAGCA | |
| | | GGGGTCTCCTTACGGAGGCAGTTTTAGCACTACAACCTGGAACTT | |
| | | GAGGGTCCTTTTTGACGAACACCAAAAACACCACAACACGTGGAG | |
| | | CTACCCCAATAACCAGCTAGACCTGGGCAGATACAAGGGCTGCAC | |
| | | CTTCTACTTTTACAGAGACAAAAAGACAGACTACATAGTAAAGTTTC | |
| | | AGAGGAGGGGACCCTTTAAAATAAACAAGTACAGCAGTCCCATGG | |
| | | CCCATCCGGGCATGATGATGCTAGATAAGATGAAAATCCTGGTGC | |
| | | CCAGCTTTGATACCAGGCCCGGGGGTCGCTGA | |
| AAX94183.1 | D0003341.1 | ATGTACTATGGCTGCATAGGAATTAATTCCACTTTAACAACCAAGTA | 226 |
| | | TGAAAACTTATTTAATAAACTATATTCCAAATGCTGCTACTTTGAAA | |
| | | CCTTTCAAACAATAGCCCAGCTAAATCCTGGCTTTAAAGCTGCTAA | |
| | | AAAGACTACTAATGGTTCTGGTTCTACAGCTGCAACACTAGGAGAC | |
| | | GCAGTAACTGAACTTAAAAACCCAAATGGTACTTTTTACACAGGCA | |
| | | ACAATAGCACCTTTGGCTGCTGCACATATAAACCCACTAAACAAAT | |
| | | AGGTAGTAATGCCAATAAGTGGTTCTGGCATCAGTTAACAGCCACA | |
| | | GATTCAGACACACTAGGCCAATACGGCCGTGCCTCCATTCAGTAT | |
| | | ATGGAGTACCACACAGGCATTTACAGCTCAATTTTTCTTAGCCCAC | |
| | | TAAGAAGCAATCTAGAACTCCCTACAGCATACCAAGATGTAACATA | |
| | | TAATCCACTAACTGACAGAGGTATAGGTAACAGAATCTGGTACCAG | |
| | | TACAGTACCAAAGAAAACACTACATTTAATGAAACACAGTGCAAAT | |
| | | GTGTACTATCAGACTTGCCACTGTGGAGCATGTTTTATGGCTATGT | |
| | | AGATTTTATAGAGTCAGAACTAGGCATCTCAGCAGAGATACACAAC | |
| | | TTTGGCATAGTATGTGTCCAGTGCCCCTACACGTTTCCCCCAATGT | |
| | | TTGACAAATCCAAACCAGATAAAGGCTACGTGTTCTATGACACCCT | |
| | | TTTTGGCAACGGAAAGATGCCAGACGGGAGCGGACACGTACCCA | |
| | | CCTACTGGCAGCAGAGGTGGTGGCCCAGATTCAGCTTCCAGAGAC | |
| | | AAGTGATGCACGACATTATCCTCACCGGGCCCTTCAGCTACAAAG | |
| | | ATGACTCTGTAATGACTGGCATAACCGCAGGCTACAAGTTTAAATT | |
| | | CTCATGGGGCGGTGATATGGTCTCCGAACAGGTCATTAAAAACCC | |
| | | AGAGAGAGGGGACGGACGAGACTCCACCTATCCCGATAGACAGC | |
| | | GCCGCGACTCACAAGTTGTTGACCCACGCTCCATGGGCCCCCAAT | |
| | | GGGTGTTCCACACCTTTGACTACAGACGGGGGCTTTTTGGAAAGG | |
| | | ACGCTATTAAGCGAGTGTCAGAAAAACCGACAGATCCTGACTACTT | |
| | | TACAACACCTTACAAAAAACCAAGATTTTTCCCTCCAACAGCAGGA | |
| | | GAAGAAAAACTGCAAGAAGAAGACTCCGCTTTACAGGAGAAAAGA | |
| | | AGCCCGCTCTCGTCAGAAGAGGGGCAGACGAGGGCGCAAGTCCT | |
| | | CCAGCAGCAGGTCCTCCAGTCGGAGCTCCAGCAGCAGCAGGAGC | |
| | | TCGGGGAGCAGCTCAGATTCCTCCTCAGGGAAATGTTCAAAACCC | |
| | | AAGCGGGCATACACATGAACCCCCGCGCATTTCAGGAGCTGTAA | |
| AAX94186.1 | DQ003342.1 | ATGTACTATGGCTGCATAGGAATTAATTCCACTTTAACAACCAAGTA | 227 |
| | | TGAAAACTTATTTAATAAACTATATTCCAAATGCTGCTACTTTGAAA | |
| | | CCTTTCAAACAATAGCCCAGCTAAATCCTGGCTTTAAAGCTGCTAA | |
| | | AAAGACTACTAATGGTTCTGGTTCTACAGCTGCAACACTAGGAGAC | |
| | | GCAGTAACTGAACTTAAAAACCCAAATGGTACTTTTTACACAGGCA | |
| | | ACAATAGCACCTTTGGCTGCTGCACATATAAACCCACTAAACAAAT | |
| | | AGGTAGTAATGCCAATAAGTGGTTCTGGCATCAGTTAACAGCCACA | |
| | | GATTCAGACACACTAGGCCAATACGGCCGTGCCTCCATTCAGTAT | |
| | | ATGGAGTACCACACAGGCATTTACAGCTCAATTTTTCTTAGCCCAC | |
| | | TAAGAAGCAATCTAGAACTCCCTACAGCATACCAAGATGTAACATA | |
| | | TAATCCACTAACTGACAGAGGTATAGGTAACAGAATCTGGTACCAG | |
| | | TACAGTACCAAAGAAAACACTACATTTAATGAAACACAGTGCAAAT | |
| | | GTGTACTATCAGACTTGCCACTGTGGAGCATGTTTTATGGCTATGT | |
| | | AGATTTTATAGAGTCAGAACTAGGCATCTCAGCAGAGATACACAAC | |
| | | TTTGGCATAGTATGTGTCCAGTGCCCCTACACGTTTCCCCCAATGT | |
| | | TTGACAAATCCAAACCAGATAAAGGCTACGTGTTCTATGACACCCT | |
| | | TTTTGGCAACGGAAAGATGCCAGACGGGAGCGGACACGTACCCA | |
| | | CCTACTGGCAGCAGAGGTGGTGGCCCAGATTCAGCTTCCAGAGAC | |
| | | AAGTGATGCACGACATTATCCTCACCGGGCCCTTCAGCTACAAAG | |
| | | ATGACTCTGTAATGACTGGCATAACCGCAGGCTACAAGTTTAAATT | |
| | | CTCATGGGGCGGTGATATGGTCTCCGAACAGGTCATTAAAAACCC | |
| | | AGAGAGAGGGGACGGACGAGACTCCACCTATCCCGATAGACAGC | |
| | | GCCGCGACTCACAAGTTGTTGACCCACGCTCCATGGGCCCCCAAT | |
| | | GGGTGTTCCACACCTTTGACTACAGACGGGGGCTTTTTGGAAAGG | |
| | | ACGCTATTAAGCGAGTGTCAGAAAAACCGACAGATCCTGACTACTT | |
| | | TACAACACCTTACAAAAAACCAAGATTTTTCCCTCCAACAGCAGGA | |
| | | GAAGAAAAACTGCAAGAAGAAGACTCCGCTTTACAGGAGAAAAGA | |
| | | AGCCCGCTCTCGTCAGAAGAGGGGCAGACGAGGGCGCAAGTCCT | |
| | | CCAGCAGCAGGTCCTCCAGTCGGAGCTCCAGCAGCAGCAGGAGC | |
| | | TCGGGGAGCAGCTCAGATTCCTCCTCAGGGAAATGTTCAAAACCC | |
| | | AAGCGGGCATACACATGAACCCCCGCGCATTTCAGGAGCTGTAA | |
| AAX94189.1 | DQ003343.1 | ATGTACTATGACTGCATAGGAATTAATTCCACTTTAACAACCAAGTA | 228 |
| | | TGAAAACTTATTTAATAAACTATATTCCAAATGCTGCTACTTTGAAA | |
| | | CCTTTCAAACAATAGCCCAGCTAAATCCTGGCTTTAAAGCTGCTAA | |
| | | AAAGACTACTAATGGTTCTGGTTCTACAGCTGCAACACTAGGAGAC | |
| | | GCAGTAACTGAACTTAAAAACCCAAATGGTACTTTTTACACAGGCA | |
| | | ACAATAGCACCTTTGGCTGCTGCACATATAAACCCACTAAACAAAT | |
| | | AGGTAGTAATGCCAATAAGTGGTTCTGGCATCAGTTAACAGCCACA | |
| | | GATTCAGACACACTAGGCCAATACGGCCGTGCCTCCATTCAGTAT | |
| | | ATGGAGTACCACACAGGCATTTACAGCTCAATTTTTCTTAGCCCAC | |
| | | TAAGAAGCAATCTAGAATTCCCTACAGCATACCAAGATGTAACATA | |
| | | TAATCCACTAACTGACAGAGGTATAGGTAACAGAATCTGGTACCAG | |
| | | TACAGTACCAAAGAAAACACTACATTTAATGAAACACAGTGCAAAT | |
| | | GTGTACTATCAGACTTGCCACTGTGGAGCATGTTTTATGGCTATGT | |
| | | AGATTTTATAGAGTCAGAACTAGGCATCTCAGCAGAGATACACAAC | |
| | | TTTGGCATAGTATGTGTCCAGTGCCCCTACACGTTTCCCCCAATGT | |
| | | TTGACAAATCCAAACCAGATAAAGGCTACGTGTTCTATGACACCCT | |
| | | TTTTGGCAACGGAAAGATGCCAGACGGGAGCGGACACGTACCCA | |
| | | CCTACTGGCAGCAGAGGTGGTGGCCCAGATTCAGCTTCCAGAGAC | |
| | | AAGTGATGCACGACATTATCCTCACCGGGCCCTTCAGCTACAAAG | |
| | | ATGACTCTGTAATGACTGGCATAACCGCAGGCTACAAGTTTAAATT | |
| | | CTCATGGGGCGGTGATATGGTCTCCGAACAGGTCATTAAAAACTC | |
| | | AGAGAGAGGGGACGGACGAGACTCCACCTATCCCGATAGACAGC | |
| | | GCCGCGACTTACAAGTTGTTGACCCACGCTCCATGGGCCCCCAAT | |
| | | GGGTATTCCACACCTTTGACTACAGACGGGGGCTTTTTGGAAAGG | |
| | | ACGCTATTAAGCGAGTGTCAGAAAAACCGACAGATCCTGACTACTT | |
| | | TACAACACCTTACAAAAAACCAAGATTTTTCCCTCCAACAGCAGGA | |
| | | GAAGAAAAACTGCAAGAAGAAGACTCCGCTTTACAGGAGAAAAGA | |
| | | AGCCCGCTCTCGTCAGAAGAGGGGCAGACGAGGGCGCAAGTCCT | |
| | | CCAGCAGCAGGTCCTCCAGTCGGAGCTCCAGCAGCAGCAGGAGC | |
| | | TCGGGGAGCAGCTCAGATTCCTCCTCAGGGAAATGTTCAAAACCC | |
| | | AAGCGGGCATACACATGAACCCCCGCGCATTTCAGGAGCTGTAA | |
| AAX94192.1 | DQ003344.1 | ATGTACTATGACTGCATAGGAATTAATTCCACTTTAACAACCAAGTA | 229 |
| | | TGAAAACTTATTTAATAAACTATATTCCAAATGCTGCTACTTTGAAA | |
| | | CCTTTCAAACAATAGCCCAGCTAAATCCTGGCTTTAAAGCTGCTAA | |
| | | AAAGACTACTAATGGTTCTGGTTCTACAGCTGCAACACTAGGAGAC | |
| | | GCAGTAACTGAACTTAAAAACCCAAATGGTACTTTTTACACAGGCA | |
| | | ACAATAGCACCTTTGGCTGCTGCACATATAAACCCACTAAACAAAT | |
| | | AGGTAGTAATGCCAATAAGTGGTTCTGGCATCAGTTAACAGCCACA | |
| | | GATTCAGACACACTAGGCCAATACGGCCGTGCCTCCATTCAGTAT | |
| | | ATGGAGTACCACACAGGCATTTACAGCTCAATTTTTCTTAGCCCAC | |
| | | TAAGAAGCAATCTAGAATTCCCTACAGCATACCAAGATGTAACATA | |
| | | TAATCCACTAACTGACAGAGGTATAGGTAACAGAATCTGGTACCAG | |
| | | TACAGTACCAAAGAAAACACTACATTTAATGAAACACAGTGCAAAT | |
| | | GTGTACTATCAGACTTGCCACTGTGGAGCATGTTTTATGGCTATGT | |
| | | AGATTTTATAGAGTCAGAACTAGGCATCTCAGCAGAGATACACAAC | |
| | | TTTGGCATAGTATGTGTCCAGTGCCCCTACACGTTTCCCCCAATGT | |
| | | TTGACAAATCCAAACCAGATAAAGGCTACGTGTTCTATGACACCCT | |
| | | TTTTGGCAACGGAAAGATGCCAGACGGGAGCGGACACGTACCCA | |
| | | CCTACTGGCAGCAGAGGTGGTGGCCCAGATTCAGCTTCCAGAGAC | |
| | | AAGTGATGCACGACATTATCCTCACCGGGCCCTTCAGCTACAAAG | |
| | | ATGACTCTGTAATGACTGGCATAACCGCAGGCTACAAGTTTAAATT | |
| | | CTCATGGGGCGGTGATATGGTCTCCGAACAGGTCATTAAAAACTC | |
| | | AGAGAGAGGGGACGGACGAGACTCCACCTATCCCGATAGACAGC | |
| | | GCCGCGACTTACAAGTTGTTGACCCACGCTCCATGGGCCCCCAAT | |
| | | GGGTATTCCACACCTTTGACTACAGACGGGGGCTTTTTGGAAAGG | |
| | | ACGCTATTAAGCGAGTGTCAGAAAAACCGACAGATCCTGACTACTT | |
| | | TACAACACCTTACAAAAAACCAAGATTTTTCCCTCCAACAGCAGGA | |
| | | GAAGAAAAACTGCAAGAAGAAGACTCCGCTTTACAGGAGAAAAGA | |
| | | AGCCCGCTCTCGTCAGAAGAGGGGCAGACGAGGGCGCAAGTCCT | |
| | | CCAGCAGCAGGTCCTCCAGTCGGAGCTCCAGCAGCAGCAGGAGC | |
| | | TCGGGGAGCAGCTCAGATTCCTCCTCAGGGAAATGTTCAAAACCC | |
| | | AAGCGGGCATACACATGAACCCCCGCGCATTTCAGGAGCTGTAA | |

In some embodiments, the genetic element comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to any one of the amino acid sequences described herein, e.g., in any of Tables 2, 4, 6, 8, 10, 12, 14, or 16. In some embodiments, the substantially non-pathogenic protein comprises a capsid protein or a functional fragment of a capsid protein or a sequence having at least about 60%, 65%, 70%, 75%, 80%, 85%, 90% 95%, 96%, 97%, 98%, 99%, or 100% sequence identity to any one of the amino acid sequences described herein, e.g., in any of Tables 2, 4, 6, 8, 10, 12, 14, or 16.

TABLE 16
Examples of amino acid sequences of substantially non-pathogenic proteins,
e.g., capsid proteins

| Accession # | Accession # | | SEQ |
|---|---|---|---|
| (nucleotide | (protein | | ID |
| sequence) | sequence) | Protein Sequence | NO: |
| AF079173.1 | AAC28465.1 | MAYGWWRRRRRRWRRWRPRPWRPRWRTRRRRPARR | 230 |
| | | RGHRRNVRRRRRGGRWRRRYRRWKRKGRRRKKAKIIIR | |
| | | QWQPNYRRRCNIVGYIPVLICGENTVSRNYATHSDDTNY | |
| | | PGPFGGGMTTDKFTLRILYDEYKRFMNYWTASNEDLDLC | |
| | | RYLGVNLYFFRHPDVDFIIKINTMPPFLDTELTAPRLHPGM | |
| | | LALDKRARWIPSLKSIPGKKHYIKIRVGAPKMFTDKWYPQ | |
| | | TDLCDMVLLTVYATAADIPYPFGSPLTDSVVVNFQVLQSM | |
| | | YDKYISILPDQKSQSKSLLSNIANYIPFYNTTQTIAQLKPFID | |
| | | AGNITSGTAATTWGSYINTTKFTTTATTTYTYPGTTTNTVT | |
| | | MYSSNDSWYRGTVYNNQIKELPKKAAELYSKATKTLLGN | |
| | | TFTTEDCTLEYHGGLYSSIWLSPGRSYFETPGAYTDIKYN | |
| | | PFTDRGEGNMLWIDWLSKKNMNYDKVQSKCLVSDLPLW | |
| | | ASAYGYVEFCAKSTGDQNIHMNARLLIRSPFTDPQLLVHT | |
| | | DPTKGFVPYSLNFGNGKMPGGSSNVPIRMRAKWYPTLF | |
| | | HQQEVLEALAQSGPFAYHSDIKEVSLGMKYRFKWIWGG | |
| | | NPVRQQVVRNPCKETHSSGNRVPRSLQIVDPKYNSPELT | |
| | | FHTWDFRRGLFGPKAIQRMQQQPTTTDIFSAGRKRPRR | |
| | | DTEVYHSSQEGEQKESLLFPPVKLLRRVPPWEDSQQEE | |
| | | SGSQSSEEETQTVSQQLKQQLQQQQILGVKLRLLFDQV | |
| | | QKIQQNQDINPTLLPRGGDLASLFQIAP* | |
| AF129887.1 | AAD20024.1 | MAYGLWRRRRRRWKRWRRRRWRRRWRTRRRRPAGR | 231 |
| | | RRRRRTVRRRRRRGRWRRRYRRWRRKGRRRKKKKLII | |
| | | RQWQPNYTRKCNIVGYMPVIMCGENTVSRNYATHSDDT | |
| | | NYPGPFGGGMTTDKFTLRILYDWYKRFMNYWTASNEDL | |
| | | DLCRYLGVNLYFFRHPDVDFIIKINTMPPFLDTELTAPSIHP | |
| | | GMLALDERARWIPSLKSRPGKKHYIKIRVGAPKMFTDKW | |
| | | YPQTDLCDMVLLTVYATAADMQYPFGYPLTDSVVVNFQV | |
| | | LQSMYDKYISILPDQKSQRESLLSNIANYIPFYNTTQTIAQL | |
| | | KPFIDAGNITSGTTATTWGSYINTTKFTTTATTTYTYPGTT | |
| | | TNTVTMLTSNDSWYRGTVYNNQIKELPKKAAELYSKATK | |
| | | TLLGNTFTTEDCTLEYHGGLYSSIWLSPGRSYFETPGAYT | |
| | | DMKYNPFTDRGEGNMLWIDWLSKKNMNYDKVQSKCLV | |
| | | SDLPLWAAAYGYLEFCSKSTGDTNIHMNARLLIRSPFTDP | |
| | | QLIAHTDPTKGFVPYSLNFGNGKMPGGSSNVPIRMRAK | |
| | | WYPTLFHQQEVLEALAQSGPFAYHSDIKKVSLGIKYRFK | |
| | | WIWGGNPVRQQVVRNPCKEPHSSVNRVPRSIQIVDPKY | |
| | | NSPELTIHAWDFRRGFFGPKAIQRMQQQPTATEFFSAGR | |
| | | KRPRRDTEVYQSDQEKEQKESSLFPPVKLLRRVPPWED | |
| | | SEQEQSGSQSSEEETHTVSQQLKQQLQQQRILGVKLRV | |
| | | LFHQVHKIQQNQHINPTLLPRGGALASLSQIAP* | |
| AF116842.1 | AAD29634.1 | MAYGLWHRRRRRWRRWKRTPWKRRWRTRRRRPARR | 232 |
| | | RGRRRNVRRRRRGGRWRRRYRRWKRKGRRRKKAKIIIR | |
| | | QWQPNYRRRCNIVGYIPVLICGENTVSRNYATHSDDTNY | |
| | | PGPFGGGMTTDKFTLRILCDEYKRFMNYWTASNEDLDLC | |
| | | RYLGVNLYFFRHPDVDFIIKINTMPPFLDTELTAPSIHPGM | |
| | | LALDKRARWIPSLKSRPGKKHYIKIRVGAPKMFTDKWYP | |
| | | QTDLCDMVLLTVYATTADMQYPFGSPLTDSVVVNFQVLQ | |
| | | SMYDKTISILPDEKSQREILLNKIASYIPFYNTTQTIAQLKPF | |
| | | IDAGNVTSGATATTWASYINTTKFTTATTTTYAYPGTNRP | |
| | | PVTMLTCNDSWYRGTVYNTQIQQLPIKAAKLYLEATKTLL | |
| | | GNNFTNEDYTLEYHGGLYSSIWLSPGRSYFETTGAYTDIK | |
| | | YNPFTDRGEGNMLWIDWLSKKNMNYDKVQSKCLVRDLP | |
| | | LWAAAYGYVEFCAKSTGDKNIYMNARLLIRSPFTDPQLLV | |
| | | HTDPTKGFVPYSLNFGNGKMPGGSSNVPIRMRAKWYPT | |
| | | LFHQQEVLEALAQSGPFAYHSDIKKVSLGMKYRFKWIWG | |
| | | GNPVRQQVVRNPCKETHSSGNRVPRSLQIVDPKYNSPE | |
| | | LTFHTWDFRRGLFGPRAIQRMQQQPTTTDILSAGRKRPR | |
| | | KDTEVYHPSQEGEQKESLLFPPVKLLRRVPPWEDSQQE | |
| | | ESGSQSSEEETQTVSQQLKQQLQQQQILGVKLRLLFDQV | |
| | | QKIQQNQDINPTLLPRGGDLASLFQIAP* | |
| AB026345.1 | BAA85662.1 | MAYGWWRRRRRRWRRWRRRPWRRRWRTRRRRPARR | 233 |
| | | RGRRRNVRRRRRGGRWRRRYRRWKRKGRRRKKAKIIIR | |
| | | QWQPNYRRRCNIVGYIPVLICGENTVSRNYATHSDDTNY | |
| | | PGPFGGGMTTDKFTLRILYDEYKRFMNYWTASNEDLDLC | |
| | | RYLGVNLYFFRHPDVDFIIKINTMPPFLDTELTAPSIHPGM | |
| | | LALDKRARWIPSLKSRPGKKHYIKIRVGAPKMFTDKWYP | |
| | | QTDLCDMVLLTVYATAADMQYPFGSPLTDSVVVNFQVLQ | |
| | | SMYDEKISILPDQKSQRESLLTSIANYIPFYNTTQTIAQLKP | |
| | | FIDAGNVTSGTTATTWGSYINTTKFTTTATTTYTYPGTTTT | |
| | | TVTMLTSNDSWYRGTVYNNQIKDLPKKAAELYSKATKTLL | |
| | | GNTFTTEDYTLEYHGGLYSSIWLSPGRSYFETPGAYTDIK | |
| | | YNPFTDRGEGNMLWIDWLSKKNMNYDKVQSKCLISDLPL | |
| | | WAAAYGYVEFCAKSTGDQNIHMNARLLIRSPFTDPQLLV | |
| | | HTDPTKGFVPYSLNFGNGKMPGGSSNVPIRMRAKWYPT | |
| | | LFHQQEVLEALAQSGPFAYHSDIKKVSLGMKYRFKWIWG | |
| | | GNPVRQQVVRNPCKETHSSGNRVPRSLQIVDPKYNSPE | |
| | | LTFHTWDFRRGLFGPKAIQRMQQQPTTTDIFSAGRKRPR | |
| | | RDTEVYHSSQEGEQKESLLFPPVKLLRRVPPWEDSQQE | |
| | | ESGSQSSEEETQTVSQQPKQQLQQQRILGVKLRLLFNQV | |
| | | QKIQQNQDINPTLLPRGGDLASLFQVAP* | |
| AB026346.1 | BAA85664.1 | MAYGWWRRRRRRWRRWRRRPWRRRWRTRRRRPARR | 234 |
| | | RGRRRNVRRRRRGGRWRRRYRRWKRKGRRRKKAKIIIR | |
| | | QWQPNYRRRCNIVGYIPVLICGENTVSRNYATHSDDTNY | |
| | | PGPFGGGMTTDKFTLRILYDEYKRFMNYWTASNEDLDLC | |
| | | RYLGVNLYFFRHPDVDFIIKINTMPPFLDTELTAPSIHPDM | |
| | | LALDKRARWIPSLKSRPGKKHYIKIRVGAPKMFTDKWYP | |
| | | QTDLCDMVLLTVYATTADMQYPFGSPLTDSVVVNFQVLQ | |
| | | SMYDENISILPTEKSKRDVLHSTIANYTPFYNTTQIIAQLRP | |
| | | FVDAGNLTSASTTTTWGSYINTTKFNTTATTTYTYPGSTT | |
| | | TTVTMLTCNDSWYRGTVYNNQISKLPKQAAEFYSKATKT | |
| | | LLGNTFTTEDHTLEYHGGLYSSIWLSAGRSYFETPGAYT | |
| | | DIKYNPFTDRGEGNMLWIDWLSKNNMNYDKVQSKCLISD | |
| | | LPLWAAAYGYVEFCAKSTGDQNIHMNARLLIRSPFTDPQ | |
| | | LLVHTDPTKGFVPYSLNFGNGKMPGGSSNVPIRMRAKW | |
| | | YPTLFHQQEVLEALAQSGPFAYHSDIKKVSLGMKYRFKW | |
| | | IWGGNPVRQQVVRNPCKETHSSGNRVPRSLQIVDPKYN | |
| | | SPELTFHTWDFRRGLFGPKAIQRMQQQPTTTDIFSAGRK | |
| | | RPRRDTEVYHSSQEGEQKESLLFPPVKLLRRVPPWEDS | |
| | | QQEESGSQSSEEETQTVSQQLKQQLQQQRILGVKLRLLF | |
| | | NQVQKIHQNQDINPTLLPRGGDLASLFQIAP* | |
| AB026347.1 | BAA85666.1 | MAYGWWRRRRRRWRRWRRRPWRRRWRTRRRRPARR | 235 |
| | | RGRRRNVRRRRRGGRWRRRYRRWKRKGRRRKKAKIIIR | |
| | | QWQPNYRRRCNIVGYIPVLICGENTVSRNYATHSDDTNY | |
| | | PGPFGGGMTTDKFTLRILYDEYKRFMNYWTASNEDLDLC | |
| | | RYLGVNLYFFRHPDVDFIIKINTMPPFLDTELTAPSIHPGM | |
| | | LALDKRARWIPSLKSRPGKKHYIKIRVEAPKMFTDKWYPQ | |
| | | TDLCDMVLLTVYATTADMQYPFGSPLTDSVVVNFQVLQS | |
| | | MYDQNISILPTEKSKRTQLHDNITRYTPFYNTTQTIAQLKP | |
| | | FVDAGNVTPVSPTTTWGSYINTTKFTTTATTTYTYPGTTT | |
| | | TTVTMLTCNDSWYRGTVYNNQISQLPKKAAEFYSKATKT | |
| | | LLGDTFTTEDYTLEYHGGLYSSIWLSAGRSYFETPGVYTD | |
| | | IKYNPFTDRGEGNMLWIDWLSKKNMNYDKVQSKCLISDL | |
| | | PLWAAAYGYVEFCAKSTGDQNIHMNAKLLIRSPFTDPQLL | |
| | | VHTDPTKGFVPYSLNFGNGKMPGGSSNVPIRMRAKWYP | |
| | | TLFHQQEVLEALAQSGPFAYHSDIKKVSLGMKYRFKWIW | |
| | | GGNPVRQQVVRNPCKETHSSGNRVPRSLQIVDPKYNSP | |
| | | ELTFHTWDFRRGLFGPKAIQRMQQQPTTTDIFSAGRKRP | |
| | | RRDTEVYHSSQEGEQKESLLFLPVKLLRRVPPWEDSQQ | |
| | | EESGSQSSEEETQTVSQQLKQQLQQQRILGVKLRLLFNQ | |
| | | VQKIQQNQDINPTLLPRGGDLASLFQIAP* | |
| AB030487.1 | BAA90406.1 | MAYGWWRRRRRRWKRWRRRPRWRRPWRTRRRRPAR | 236 |
| | | RRGRRRTVRRRERGRWRRRYRRWRKKGKRRIKKKLIIR | |
| | | QWQPNYTRKCDILGYMPVIMCGENTLIRNYATHANDCY | |
| | | WPGPFGGGMATQKFTLRILYDDYKRFMNYVVTSSNEDLD | |
| | | LCRYRGVTLYFFRHPDVDFIILINTTPPFVDTEITGPSIHPG | |
| | | MMALNKRARFIPSLKTRPGRRHIVKIRVGAPKLYEDKWYP | |
| | | QSELCDMPLLTVYATAADMQYPFGSPLTDTPVVTFQVLR | |
| | | SMYNDALSILPSNFEQDDNAGQKLYNEISSYLPYYNTTET | |
| | | IAQLKRYVENTEKISTTPNPWQSNYVNTITFTTAQSITTTT | |
| | | PYTTFSDSWYRGTVYKNAITKVPLAAAKLYETQTKNLLSP | |
| | | TFTGGSEYLEYHGGLYSSIWLSAGRSYFETKGAYTDICY | |
| | | NPYTDRGEGNMLWIDWLSKGDSRYDKARSKCLIEKLPM | |
| | | WAAVYGYAEYCAKATGDSNIDMNARVVMRCPYTVPQMI | |
| | | DTSDPLRGFIPYSFNFGKGKMPGGTNQVPIRMRAKWYP | |
| | | CLFHQKEVLEAIGQSGPFAYHSDQKKAVLGLKYRFHWIW | |
| | | GGNPVFPQVVRNPCKDTQGSTGPRKPRSVQIIDPKYNTP | |
| | | ELTIHAWDFRRGFFGPKAIKRMQQQPTDAELLPPGRKRS | |
| | | RRDTEVLQSSQERQKESLLLQQLHLQGRVPPWESLQGL | |
| | | QTETESQKEHEGTLSQQIREQVQQQKLLGRQLREMFLQ | |
| | | LHKILQNQHVNPTLLPRDQGLIWWFQIQ* | |
| AB030488.1 | BAA90409.1 | MAYGWWRRRRRRWKRWRRRPRWRRPWRTRRRRPAG | 237 |
| | | RRGRRRTVRRRRRGRWRRRYRRWRKKGRRRRKKKLII | |
| | | RQWQPNYTRKCNIVGYMPVIMCGENTLIRNYATHAYNCS | |
| | | WPGPFGGGMATQKFTLRILYDDYKRFMNYVVTSSNEDLD | |
| | | LCRYRGATLYFFRDPDVDFIILINTTPPFVDTEITGPSIHPG | |
| | | MLALNKRARFIPSLKTRPSRRHIVKIRVGAPKLYEDKWYP | |
| | | QSELCDMPLLTVYATATDMQYPFGSPLTDTPIVTFQVLRS | |
| | | MYNDALSILPSNFEGDDSAGAKLYKQISEYIPYYNTTETIA | |
| | | QLKGYVENTEKTQTTPNPWQSKYVNTKPFDTAQTITNQK | |
| | | PYTPFADTWYRGTAYKEEIKNVPLKAAELYELHTTHLLST | |
| | | TFTGGSKYLEYHGGLYSSIWLSAGRSYFETKGAYTDICY | |
| | | NPYTDRGEGNMVWIDWLVKTDSRYDKTRSKCLIEKLPLW | |
| | | AAVYGYAEYCAKATGDSNIDMNARVVIRSPYTTPQMIDT | |
| | | NDSLRGFIVYSFNFGKGKMPGGTNQVPIRMRAKWYPCL | |
| | | FHQKEVLEAIGQSGPFAYHSDQKKAVLGLKYRFHWIWG | |
| | | GNPVFPQVVRNPCKDTQGSTGPRKPRSVQIIDPKYNTPE | |
| | | LTIHAWDFRRGFFGPKAIKRMQQQPTDAELLPPGRKKSR | |
| | | RDTEVLQSSQERQKESLLFQQLQLQRRVPPWESSQGSQ | |
| | | TETESQKEQEGTLSQQLREQLQQQKLLGRQLREMFLQIH | |
| | | KILQNQQVNPILLPRDQALISWFQIQ* | |
| AB030489.1 | BAA90412.1 | MAYGWWRRRRRRWKRWRRRPRWRRRWRTRRRRPAG | 238 |
| | | RRRRRRTVRRRRRGRWRSRYRRWRRKGRRRRKEKLII | |
| | | RQWQPNYTRKCNIVGYMPVIMCGENTVIRNYATHTYDCS | |
| | | WPGPFGGGMATQKFTLRILYDDYKRFMNYVVTSSNEDLD | |
| | | LCRYRGATLYFFRDPDVDFIILINTTPPFVDTEITGPSIHPG | |
| | | MLALNKRARFIPSLKTRPGRRHIVKIKVGAPRMYEDKWYP | |
| | | QSELCDMPLLTIYATATDMQHPFGSPLTDTPVVTFQVLRS | |
| | | MYNDALSILPSNFEDDSSPGAALYKQISEYIPYYNTTETIA | |
| | | QLKRYVENTEKTQTTLNPWQSRYVNTTLFNTAETIANQK | |
| | | PYTKFADTWYRGTAYKDAIKDIPLKAAELYVNQTKYLLST | |
| | | TFTGGSKYLEYHGGLYSSIWLSAGRSYFETKGAYTDICY | |
| | | NPYTDRGEGNMVWIDWLSKTDSKYDKTRSKCLIEKLPLW | |
| | | ASVYGYAEYCAKATGDSNIDMNARVVIRCPYTTPQMIDTT | |
| | | DPTRGFIVYSFNFGKGKMPGGSNEVPIRMRAKWYPCLF | |
| | | HQKEVLEAIGQSGPFAYHSDQKKAVLGLKYKFHWIWGG | |
| | | NPVFPQVIKNPCKNTQFSTGPRKPRSLQIIDPNYNTPKLTI | |
| | | HAWDFRLGFFGPKAIKRMQQQPTDAELLPPGRKRSRRD | |
| | | TEVLQSSQERQKGNLLFQQFQLQRRVPPWESSQGSQT | |
| | | GTQSQKEQEGTLSQQLREQLQQQKLLGRQLREMFLQLH | |
| | | KIQQNQHVNPTLLPRDQALICWFQIQ* | |
| AB038340.1 | BAA90825.1 | MAYGWWRRRRRRWRRWRRRPWRRRWRTRRRRPARR | 239 |
| | | RGRRRNVRRRRRGGRWRRRYRRWKRKGRRRKKAKIIIR | |
| | | QWQPNYRRRCNIVGYIPVLICGENTVSRNYATHSDDTNY | |
| | | PGPFGGGMTTDKFTLRILYDEYKRFMNYVVTASNEDLDLC | |
| | | RYLGVNLYFFRHPDVDFIIKINTMPPFLDTELTAPSIHPGM | |
| | | LALDKRARWIPSLKSRPGKKHYIKIRVGAPKMFTDKWYP | |
| | | QTDLCDMVLLTVYATAADMQYPFGSPLTDSVVVNFQVLQ | |
| | | SMYDEKISILPDQKSQRESLLTSIANYIPFYNTTQTIAQLKP | |
| | | FIDAGNVTSGTTATTWGSYINTTKFTTTATTTYTYPGTTTT | |
| | | TVTMLTSNDSWYRGTVYNNQIKDLPKKAAELYSKATKTLL | |
| | | GNTFTTEDYTLEYHGGLYSSIWLSPGRSYFETPGAYTDIK | |
| | | YNPFTDRGEGNMLWIDWLSKKNMNYDKVQSKCLISDLPL | |
| | | WAAAYGYVEFCAKSTGDQNIHMNARLLIRSPFTDPQLLV | |
| | | HTDPTKGFVPYSLNFGNGKMPGGSSNVPIRMRAKWYPT | |
| | | LFHQQEVLEALAQSGPFAYHSDIKKVSLGMKYRFKWIWG | |
| | | GNPVRQQVVRNPCKETHSSGNRVPRSLQIVDPKYNSPE | |
| | | LTFHTWDFRRGLFGPKAIQRMQQQPTTTDIFSAGRKRPR | |
| | | RDTEVYHSSQEGEQKESLLFPPVKLLRRVPPWEDSQQE | |
| | | ESGSQSSEEETQTVSQQPKQQLQQQRILGVKLRLLFNQV | |
| | | QKIQQNQDINPTLLPRGGDLASLFQVAP* | |
| AB038622.1 | BAA93586. | TAWWWGRWRRRWRPRYRRRTWRVRRRRPRRTFRRR | 240 |
| | | RRGRYVSRRRRRRYYRRRLRRGRRRGRRKRHRQTLVL | |
| | | RQWQPDIVRHCKITGWMPLIICGSGSTQNNFITHMDDFP | |
| | | PMGYSFGGNFTNLSFSLEGIYEQFLYHRNRWSRSNHDL | |
| | | DLARYKGTTLKLYRHHTLDYIVSYNRTGPFQISDMTYLST | |
| | | HPALMLLQKHRIVVPSLLTKPKGKRSIKVRIKPPKLMLNK | |
| | | WYFTKDICSMGLFQLQATACTLYNPWLRDTTKSPVIGFR | |
| | | VLKNSIYTNLSNLPEHDQTRQAIRRKLHPDSLTGSTPYQK | |
| | | GWEYSYTKLMAPIYYQANRNSTYNWLNYQTNYAQTFTK | |
| | | FKEKMNENLALIQKEYSYHYPNNVTTDLIGKNTLTHDWGI | |
| | | YSPYWLTPTRISLDWETPWTYVRYNPLADKGIGNAVYAQ | |
| | | WCSEQTSKLDTKKSKCIMKDLPLWCIFYGYVDWIIKSTGV | |
| | | SSAVTDMRVAIISPYTEPALIGSSPDVGYIPVSDTFCNGD | |
| | | MPFLAPYIPVGWWIKWYPMIAHQKEVFEAIVNCGPFVPR | |
| | | DQTTPSWEITMGYKMDWLWGGSPLPSQAIDDPCQKPTH | |
| | | ELPDPDRHPRMLQVSDPTKLGPKTVFHKWDWRRGMLS | |
| | | KRSIKRVQEDSTDDEYVAGPLPRKRNKFDTRAQGLQTPE | |
| | | KESYTLLQALQESGQETSSEDQEQAPQEKEGQKEALME | |
| | | QLQLQKQHQRVLKRGLKLLLGDVLRLRRGVHWDPLLS* | |
| AB038623.1 | BAA93589.1 | TAWWWGRWRRRWRPRYRKRTWRLRRRRPRRTFRRR | 241 |
| | | RRRQYVSRRRRRRYYRRRLRRGRRRGRRKRHRQTLVL | |
| | | RQWQPDVVRHCKITGWMPLIICGSGSTQNNFITHMDDFP | |
| | | PMGYSFGGNFTNLTFSLEGIYEQFLYHRNRWSRSNHDL | |
| | | DLARYKGTTLKLYRHHTLDYIVSYNRTGPFQISDMTYPST | |
| | | HPALMLLQKHRIVVPSVLTKPKGKRSIKVRIKPPKLMLNK | |
| | | WYFTKDICSMGLFQLQATACTLYNPWLRDTTKSPVIGFR | |
| | | VLKNSIYTNLSNLPDHEGSREAIRKKLHPQSLTGHSPNQK | |
| | | GWEYSYTKLMAPIYYSANRNSTYNWLNYQDNYVATYTK | |
| | | FKVKMTDNLQLIQKEYSYHYPNNTTTDLIKNNTLTHDWGI | |
| | | YSPYWLTPTRISLDWETPWTYVRYNPLADKGIGNAVYAQ | |
| | | WCSEQTSKLDPKKSKCIMRDLPLWCIFYGYVDWIVKSTG | |
| | | VSSAVTDMRVAIRSPYTEPALIGSTEDVGFIPVSDTFCNG | |
| | | DMPFLAPYIPVGWWIKWYPMIAHQKEVFEQIVNCGPFVP | |
| | | RDQTTPSWEITMGYKMDWLWGGSPLPSQAIDDPCQKPT | |
| | | HELPDPDRHPRMLQVSDPTKLGPKTVFHRWDWRRGML | |
| | | SKRSIKRVQEDSTDDEYVAGPLPRKRNKFDTRAQGLQSP | |
| | | EKESYTLLQALQESGQESSSEDQEQAPQEKEGQKEALM | |
| | | EQLQLQKQHQRVLKRGLKLLLGDVLRLRRGVHWDPLLS* | |
| AB038624.1 | BAA93592.1 | TAWWWGRWRRRWRPRYRRRTWRVRRRRPRRTFRRR | 242 |
| | | RRGRYVSRRRRRRYYRRRLRRGRRRGRRKRHRQTLVL | |
| | | RQWQPDVLRRCKITGWMPLIICGSGSTQNNFITHMDDFP | |
| | | PMGYSYGGNFTNLTFSLEGIYEQFLYHRNRWSRSNHDL | |
| | | DLARYKGTTLKLYRHHTLDYIVSYNRTGPFQISDMTYLST | |
| | | HPALMLLQKHRIVVPSLLTKPKGKRSIKVRIKPPKLMLNK | |
| | | WYFTKDICSMGLFQLQATACTLYNPWLRDTTKSPVIGFR | |
| | | VLKNSIYTNLSNLPDHEGAREAIRKKLHPQSLTGSVPNQK | |
| | | GWEYSYTKLMAPIYYQAIRNSTYNWLNYQQNYSQTYQTF | |
| | | KQKMQDNLQLIQKEYMYHYPNNVTTDILGKNTLTHDWGI | |
| | | YSPYWLTPTRISLDWETPWTYVRYNPLADKGIGNAVYAQ | |
| | | WCSEQTSNLDTKKSKCIMKDLPLWCIFYGYVDWVVKSTG | |
| | | VSSAVTDMRVAIISPYTEPALIGSSPEVGYIPVSDTFCNGD | |
| | | TPFLAPYIPVGWWIKWYPMIAHQKEVFEAIVNCGPFVPRD | |
| | | QTTPSWEITMGYKMDWLWGGSPLPSQAIDDPCQKPTHE | |
| | | LPDPDRHPRMLQVSDPTKLGPKTVFHKWDWRRGMLSK | |
| | | RSIKRVQEDSTDDEYVAGPLPRKRNKFDTRAQGLQSPEK | |
| | | ESYTLLQALQESGQETSSEDQEQAPQEKEGQKEALMEQ | |
| | | LQLQKQHQRVLKRGLKLLLGDVLRLRRGVHWDPLLS* | |
| AF254410.1 | AAF71533.1 | MAQGRRRYRRGWQRRVYLRRRRRRRRKRLVLTQWHP | 243 |
| | | AVRRKCTITGYMPVVWCGHGRASYNYAWHSDDCIKQP | |
| | | WPFGGSLSTVSFNLKVLYDENQRGLNRWTYPNDQLDLG | |
| | | RYKGCKLTFYRTKNTNYPGPFGGGMTTDKFTLRILYDEY | |
| | | KRFMNYWTASNEDLDLCRYLGVNLYIFRHPDVDFIIKINT | |
| | | MPPFLDTEITAASIHPGILALDKRARWIPSLKSRPGKKHYI | |
| | | KIRVGAPKMFTDKWYPQTDLCDMVLLTIYATAADMQYPF | |
| | | GSPLTDTVVVNFQVLQSMYDENISILPDQKTQREKLLTSIS | |
| | | NYIPFYNTTQTIAQLKPFVDAGNKVSGTTTTTWASYINTT | |
| | | RFTTTATTTYTYPGSTTNTVTMLTSNDSWYRGTVYNNQI | |
| | | KNLPKQAAELYSKATKTLLGNTFTTEDYTLEYHGGLYSSI | |
| | | WLSPGRSYFETPGAYTDIKYNPFTDRGEGNMLWIDWLS | |
| | | KKNMNYDKVQSKCLVSDLPLWAAAYGYVEFCAKSTGDQ | |
| | | NIHMNARLLIRSPFTDPQLLVHTDPTKAFVPYSLNFGNGK | |
| | | MPGGSSNVPIRMRAKWYPTLFHQQEVLEALAQSGPFAY | |
| | | HSDIKKVSLGIKYRFKWIWGGNPVRQQVVRNPCKEPHSS | |
| | | GNRVPRSIQIVDQKYNSPELTIHSWDFRRGFFGPKAIQR | |
| | | MQQQPTATEFFSAGRKRPRRDTEVYQSDQEKEQKESSL | |
| | | FPPVKLLRRVPPWEDSDRKQSGSQSSEEETQTVSQQLK | |
| | | QQLQQQRILGVKLRLLFYQIQRIQQNQDINPTLLPRGGDL | |
| | | ASLFQIA* | |
| AB050448.1 | BAB19928.1 | MAWIWWWQRRRRRWPWRRRRWRRLRTRRPRRLVRR | 244 |
| | | RRKRYRVRRRRRWGRRRGRRTYLRRGLKKRKRRKKLR | |
| | | LTQWNPSTIRGCTIKGMAPLIVCGHTMAGNNFAIRMEDY | |
| | | VSQIKPFGGSFSTTTWSLKVLWDEHTRFHNTWSYPNTQL | |
| | | DLARFKGVTFYFYRDKDTDFIITYSSVPPFKIDKYSSAMLH | |
| | | PGMLMQRKKKILLPSFTTRPRGRKKVKVHIKPPVLFEDK | |
| | | WYTQQDLCDVNLLSLAVSAASFRHPFCPPQTDNICITFQV | |
| | | LKDKYYTQMSVTPDTAGTKKDDEILDHLYSTAEYYQTVH | |
| | | TQGIINKTQRVAKFSTSNNTLGDQSEISLYLNQPTTTNIGN | |
| | | TLSTGHNSVYGFPSYNPQKDKLRKIADWFWTQEANKEN | |
| | | VVTGSYSMPTNKAVGYHLGKYSPIFLSSYRTNLQFRTAY | |
| | | TDVTYNPLNDKGKGNEIWVQYVTKPDTVFNPTQCKCHVI | |
| | | DLPLWSAFHGYIDFVQSELGIQEEILNIAIIVVICPYTKPKLV | |
| | | HETNPKQGFVFYDTQFGDGKMPEGSGLVPIYYQNRWYP | |
| | | RIKFQSQVVHDFILTGPFSYKDDLKSTVLTVEYKFKFLWG | |
| | | GNMIPEQVIRNPCKTEGHDLPHTSRLHRDLQVVDPHTVG | |
| | | PQWALHTWDWRRGLFGSEAIKRVSEQQVHDELYYPPSK | |
| | | KPRFLPPISGLQEQERDYSSQEEKEQSSSEEETDPKKKE | |
| | | QKQQQRLHLQFQEQQRLGNQLRLIFRELQKTQAGLHLN | |
| | | PMLSNRL* | |
| AY026465.1 | AAK01940.1 | MAWGWWKRRRRWWFRKRWTRGRLRRRWPRPARRRP | 245 |
| | | RRRRVRRRRRWRRGRPRRRLYRRYRRKKRRRRKPKIIL | |
| | | KQWQPDIVKRCYIVGYIPAIICGAGTWSHNYTSHLLDIIPK | |
| | | GPFGGGHSTMRFSLKVLFEEHLRHLNFWTRSNQDLELV | |
| | | RYFRCSFRFYRDQHTDYLVHYNRKTPLGGNRLTAPSLHP | |
| | | GVQMLSKNKIIVPSYDTKPKGKSYVKVTIAPPTLLTDKWY | |
| | | FAKDVCDTTLVNLDVVLCNLRFPFCSPQTDNPCITFQVLH | |
| | | SIYNDFLSIVDTQEYKNNFVTTLSTKLGTTWGSRLNTFRT | |
| | | EGCYSHPKLPKKQVTAANDSTYFTQPDGLWGDAVFETK | |
| | | DTTIITKNMESYATSAKQRGVNGDPAFCHLTGIYSPPWLT | |
| | | PGRISPETPGLYTDVTYNPYADKGVGNRIWVDYCSKKGN | |
| | | KYDNTSKCLLEDMPLWMVTFGYVDWVKKETGNWGIPLW | |
| | | ARVLIRSPYTVPKLYNEADPSYGWVPISYYFGEGKMPNG | |
| | | DMYVPFKVRMKWYPSMWNQEPVLNDLAKSGPFAYKDT | |
| | | KTSVTVTTKYKFTFNFGGNPVPSQIVQDPCTQPTYDIPGT | |
| | | GNLPRRIQVIDPKVLGPHYSFHRWDFRRGLFGQQAIKRV | |
| | | SEQQTTSEFLFSGPKRPRIDQGPYIPPEKGSDSLQRESR | |
| | | PWSTSESEAETEAPSEEEPENQEEQVLQLQLRQQLREQ | |
| | | RKLRQGIQCLFEQLITTQQGVHKNPLLE* | |
| AY026466.1 | AAK01942.1 | MAYGWWARRRRRWRRWKRRPWRRRWRTRRRRPRRR | 246 |
| | | YRRRRHVRRRRRGRWRRRYRKWRRKGRRRGKKKIIIRQ | |
| | | WQPNYRRRCNIIGYMPVLICGNNTVSRNYATHSDDSYLP | |
| | | GPFGGGMTTDKFTLRILYDEYCRFMNYWTASNEDLDLC | |
| | | RYRGCTLWFFRHPDVDFIILINTMSPFLDTQLTGPSIHPGL | |
| | | MALNKRARWIPSLKSRPGRKHVVKIRVGAPRMFTDKWY | |
| | | PQSDLCDLPLLTIFASAADMQYPFGSPLTDSVVVGFQVL | |
| | | QSMYNDCLSILPENFNGNGKGKALHDNITKYLPNYNTTQ | |
| | | TLAQLKPYIDNTSTGSTNNWSSYVNTSKFTTASKTITTSA | |
| | | EGPYYTFADTWYRGTAYNNSITNVPLQAAQLYHDTTKKL | |
| | | LGTTFTGGSPYLEYHGGLYSSIWLSAGRSYFETKGTYTDI | |
| | | TYNPFTDRGQGNMVWIDWVSKYDSVYSKTQSKCLIENLP | |
| | | LWASVYGYAEYCSKSTGDTNIEQNCRVVIRSPFTNPQLL | |
| | | DHNNPLRGYVPYSINFGNGKMPGGSSQVPIRMRSKWYP | |
| | | TLFHQKEVLEAIAQAGPFAYHSDQMKVSLGMKYAFKWV | |
| | | WGGNPVSQQVVRNPCKDTGVSSGNRVPRSVQIVDPKY | |
| | | NTPELAIHAWDFRRACLAQKLLRECKQNRTLLNFFRQGE | |
| | | KDTGETQKLYSPAKKNNKKKTYFSSQSSSSDQSPVGGV | |
| | | GPKPKRGRGGPTRDADTLPAAPAAAQGAAAHGGPTPSP | |
| | | VPTITTGPTKHTYRPYLFARGAGVTSLFQTA* | |
| AF345521.1 | AAK11696.1 | MAWWGRWRRWPRRRWRRWRRRRRRRLPTRRTRRAV | 247 |
| | | RGLGRRPRKTVRRRRRRPRRTYRRGWRRRRYIRRRRG | |
| | | RRKKLTLTMWNPNIVRRCNIEGGLPLILCGENRAAFNYAY | |
| | | HSEDYTEQPFPFGGGMSTTTFSLRGLYDQYTKHMNRWT | |
| | | FSNDQLDLARYRGCKFRFYRHPTCDFIVHYNLVPPLKMN | |
| | | QFTSPNTHPGLLMLTKHKIIIPSFLTRPGGRRFVKIRLPPP | |
| | | KLFEDKWYTQQDLCKQPLVTLTATAASLRYPFCSPQTNN | |
| | | PNCTFQVLRKNYHKVIGTSSTNSEDVTPFENWLYNTASH | |
| | | YQTFATEAQVGRIPSFNPDGTKNTKESEWQNYWSKKGE | |
| | | PWNPNSSYPHTTTNQMYKIPFDSNYGFPTYKPIKEYMLQ | |
| | | RRAWSFKYETDNPVSKKIWPQPTTTKPTIDYYEYHAGWF | |
| | | SNIFIGPNRHSLQFQTAYVDTTYNPLNDKGKGNKIWFQY | |
| | | HSKVNTDLRDRGIYCLLEDMPLWSMTFGYSDYVSTQLGP | |
| | | NVDHETQGLVCIICPYTEPPMYDKTNPNSGYVAYDTNFG | |
| | | NGKMPSGRSQVPVYWQCRWRPMLWFQQQVLNDISKS | |
| | | GPYAYRDELKNCCLTAYYNFIFDWGGDMYYPQVIKNPCA | |
| | | DSGLVPGTSRFTREVQVVSPLSMGPQYILHLFDQRRGFF | |
| | | SSNALKRMQQQQEFDESFTVKPKRPKLSTAAHVEQQEE | |
| | | DSSSRERKSGSSQEEVQEEVLQTPEIQLHLQRNIREQLHI | |
| | | KQQLQLLLLQLFKTQANIHLNPRFISP* | |
| AF345522.1 | AAK11698.1 | MAWRRWRWRPWWRRRRRRRWRRRRRRPRRRRPYR | 248 |
| | | RRRPRRVRRRRGRWRRAYRRWGRRRRRRRHKKKLVLT | |
| | | QWQPAVVKRCLIVGFDPLIICGINRTIFNYTTHSEDFTFNN | |
| | | DSFGGGLCTAQYTLRILFQEKLAQHNFWSASNEDLDLAR | |
| | | YLGATIVLYRHPTVDFLVRIRTSPPFEDTDMTAMTLHPGM | |
| | | MMLAKKTIKIPSLKTRPSRKHVVRIRVGAPKLFEDKWYPQ | |
| | | NELCDVTLLTIQATTADFQYPFGSPLTNSPCCNFQVLNSN | |
| | | YDNAHSILNLSNEPTNKWHTYRNNCYKFLLEQYSYYNTK | |
| | | QVVAQLKYKWNPNQNPTMPNTSNASLSKKPDDLTKTKT | |
| | | TNEYPHWDTLYGGLAYGHSTVTPGTTSSPTDLKTQMLT | |
| | | GNEFYTTAGKKLIDTFHPIPYYENGSSKANTNIFDYYTGM | |
| | | YSSIFLSSGRSNPEVKGSYTDISYNPLTDKGVGNMIWIDW | |
| | | LTKGDTVYDPKKSKCLLSDFPLWSLCYGYPDYCRKQTG | |
| | | DSGIYYDYRVLIRCPYTYPQLIKHNDKYFGFVVYSENFGL | |
| | | GRLPGGNPNPPTRMRLHWYPNMFHQTEVLECIAQSGPF | |
| | | AYHGDERKAVLTAKYKFRWKWGGNPVFQQVLRDPCTG | |
| | | GAVAPHTSRHPRAIQVHDPKYQAPEYLFHKWDFRRGLF | |
| | | STKGIKRVSEQPVHDEYFTGSSKRPKKDTNPSPQGEEQK | |
| | | EGSRFRVPELRPWLPSSQETQSQSEQEETAPKTVQEQL | |
| | | QEQLQQQQLMGIQLRNVCLQLARVQAGHSLHPVFQCHA | |
| AF345525.1 | AAK11704.1 | MAWGWWRRRRKWWWRRRFARSRLRRRRIRRPRRRTR | 249 |
| | | RRTVRRRRQWRRGRPRRRLFKRKRRFKRRRRKAKIKIT | |
| | | QWQPSSVKRCFVIGYFPLVICGPGRWSENFTSHIEDKISK | |
| | | GPFGGGHSTSRWSLKVLYEEFQRHHNFWTRSNKDLELV | |
| | | RFFGSSWRFYRHEDTDYIVYYSRKAPLGGNLLTAPSLHP | |
| | | GAAMLSKHKIVVPSFKTRPGGKPTVKINIKPPTTLIDKWYF | |
| | | QKDICDTTFLNLNVVLONLRFPFCSPQTDNICVTFQILHEV | |
| | | YHNYISITAKELLTGTEWRQYYKNFLNAALPNDRSVNKLN | |
| | | TFSTEGAYSHPQIKKHTENITGSGDKYFRKKDGLWGDAI | |
| | | HITDQQNRTEVIDLILKNAENYLKKVQQEYQGQENLKNLI | |
| | | HPVFCQYVGIFGQPTTKLPQNKPRNSRPVQRHNI* | |
| AF345527.1 | AAK11708.1 | MSWWGWRRRWWWKPRRRWRRRRARRPRRLPRRRY | 250 |
| | | RRPTRRYRGRRVRRRRAGGWRGRRRYSRRYSRRLTVR | |
| | | RKKKKLTLKIWQPQNIRRCKIRGLLPLLICGHTRSAFNYAI | |
| | | HSDDKTPQQQSFGGGLSTVSFSLKVLFDPNQRGLNRWS | |
| | | ASNDQLDLARYTGCTFWFYRHKKTDFIVQYDVSAPFKLD | |
| | | KNSCPSYHPFMLMKAKHKVLIPSFDTKPKGREKIKLRIQP | |
| | | PKMFIDKWYTQEDLCPVILVTLVATAASFTHPFCSPQTAN | |
| | | PCITFQVLKEFYYQAMGYGTPETTMSTIWNTLYTTSTYW | |
| | | QSHLTPQFVRMPKNNPDNTANTEANKFNEWVDKTFKTG | |
| | | KLVKYNYNQYKPDIEKLTLLRQYYFRWETQHTGVAVPPT | |
| | | WTTPTTDRYEYHVGMFSPIFLTPYRSAGLDFPYAYADVT | |
| | | YNPLTDKGVGNRMWYQYNTKIDTQFDAKCCKCVLEDMP | |
| | | LYAMAFGHADFLEQEIGEYQDLEANGYVCVISPYTKPPM | |
| | | FNKHNPQQGYVFYDSQWGNGKWIDGTGFVPVYWLTRW | |
| | | RVELLFQKQVLSDLAMSGPFSYPDELKNTVLTAKYRFDF | |
| | | KWGGNLFHQQTIRNPCKPEETSTGRIPRDVQVVDPVTM | |
| | | GPRFVFHSWDWRRGFLSDRALKRMFEKPLDFEGFTATP | |
| | | KRPRILPPTEGQLAREQKEQEESSDSQEESSLTPLEEVP | |
| | | QETKLRLHLRKQLREQRSIRHQLRTMFQQLVKTQAGLHL | |
| | | NPLLSSQL* | |
| AF345528.1 | AAK11710.1 | MWNPSTIRACNIKGAINLVMCGHTQAGRNYAIRSEDFYP | 251 |
| | | QIQSFGGSFSTTTWSLRVLFDEYQKFHNFWTYPNTQLDL | |
| | | CRYKYAIFTFYRDPKVDYIVIYNTNPPFKINKYSSPFLHPG | |
| | | LMMLQKKKILIPSFQTKPGGKSRIKVKIKPPALFEDKWYTQ | |
| | | QDLCPVNLLSLAVSACSFIHPFCSPESDTICMTFQVLREF | |
| | | YYTHLTVTPTTTTSTPEKDKKIFNDQLYSNANFYQSLHAS | |
| | | AFLNIAQAPAIHGHNGIPNNSRYLSSTGTETSFRTGNNSIY | |
| | | GQPNYKPIPEKLTEIRKWFFKQATTPNEIHGTYGKPTYDA | |
| | | VDYHLGKYSPIFLSPYRTNTQFPTAYMDVTYNPNVDKGK | |
| | | GNKIWLQSVTKETSDFDSRSCRCIIENLPMWAMVNGYSD | |
| | | FAESELGSEVHAVYVCCIICPYTKPMLYNKTNPAMGYIFY | |
| | | DTLFGDGKLPSGPGLVPFYWQSRWYPKLAWQQQVLHD | |
| | | FYLCGPFSYKDDLKSFTINTTYKFKFLWGGNMIPEQVIKN | |
| | | PCKTTDPTYTLSDRQRRDLQVVDPITMGPQWEFFITANDW | |
| | | RRGLFGQNALRRVSEKPGDDAEYYAPPKKPRFFPPTDLE | |
| | | EQEKDSDSQEETRLLFHPSPPRSQEEIQQEQQRDIHLRL | |
| | | GQQLRIRQQLQQVFLQVLKTQANLHINPLFLNQQ* | |
| AF345529.1 | AAK11712.1 | MAWGWWRRWRRWPTRRWRRRRRRRPVRRTRARRPA | 252 |
| | | RRYRRRRTVRTRRRRWGRRRYRRGWRRRTYVRKGRH | |
| | | RKKKKRLVLRQWQPATRRRCTITGYLPIVFCGHTKGNKN | |
| | | YALHSDDYTPQGQPFGGALSTTSFSLKVLYDQHQRGLN | |
| | | KWSFPNDQLDLARYRGCKFYFYRTKQTDWVGQYDISEP | |
| | | YKLDKYSCPNYHPGNMIKAKHKFLIPSYDTNPRGRQKIIV | |
| | | KIPPPDLFVDKWYTQEDLCDVNLVSFAVSAASFLHPFGS | |
| | | PQTDNPCYTFQVLKEFYYQAIGFSATEEKIQNVFNILYEN | |
| | | NSYWESNITPFYVINVKKGSNTAQYMSPQISDADFRNKV | |
| | | NTNYNWYTYNAKTHKEKLKTLRQAYFKQLTSEGPQHTSS | |
| | | HAGYATQWTTPSTDAYEYHLGMFSTIFLAPDRPVPRFPC | |
| | | AYQDVTYNALMDKGVGNHVWFQYNTKADTQLILTGGSC | |
| | | KAHIENIPLWAAFYGYSDFIESELGPFVDAETVGLICVICP | |
| | | YTKPPMYNKTNPMMGYVFYDRNFGDGKWTDGRGKIEP | |
| | | YWQVRWRPEMLFQETVMADIVQTGPFSYKDELKNSTLV | |
| | | CKYKFYFTWGGNVMFQQTIKNPCKTDEQPTDSGRHPRG | |
| | | IQVADPEQMGPRWVFHSFDWRRGYLSEKALKRLQEKPL | |
| | | DYDEYFTQPKRPRMFPPTESAEGEFREPEKGSYSEEER | |
| | | SQASAEEQTKEATVLLLKRRLREQQQLQQQLQFLTREMF | |
| | | KTQAGLHLNPMLLNQR* | |
| AF371370.1 | AAK54731.1 | MRFSRIYRPKKGPLPLPLVRAEQKKQPSDMSWRPPLHN | 253 |
| | | GAGIERQFFEGCFRFHASCCGCGNFVTHITLLAARYGFT | |
| | | GGPTPPGGPGALPSLRRALPPPPAPQDQAEPELWRGRG | |
| | | GGGEGNAGGRAEGGDGEGYEPEELEELFRAAAADDE* | |
| AB060596.1 | BAB69916.1 | MAFRWWWWRRRPQRRWTRRRWRRLRTRRPRRTVRR | 254 |
| | | RRRRPRVRRRRWGRRRGRRRLYRRTYRKRRKRRKKMT | |
| | | LKMWNPSTIRACNIRGFIALVVCGHTRAGCNYAIHSEDYI | |
| | | PQLRPYGGSFSTTTWSLKLLFDEYLKFRNKWSYPNTELN | |
| | | LARYRGATFTFYRDPKVDYIVVYNTVPPFKLNKYSCPMLH | |
| | | PGMMMQYKKKVLIPSYQTKPKGKAKIRLRIKPPVLFEDK | |
| | | WYTQQDLCPVNLLSLAVSACSFLHPFIPPESDNICITFQVL | |
| | | RDFYYTQMSVTPTTTTSLNQKDEKIFSDHLYKNPEYWQS | |
| | | HHTAARLSTSQKPALRNKEEIPNDHGYLNTTPTDSTFRT | |
| | | GNNTIYGQPSYRPNYTKLTKIREWYFTQENTDNPINGSYL | |
| | | KPTLNSVDYHLGKYSAIFLSPYRTNTQFDTAYQDVTYNPN | |
| | | TDKGKGNKIWIQSCTKESTILDNACRCVIEDMPLWAMVN | |
| | | GYLEFCDSELPGANIYNTYIVVVICPYTKPQLLNKTNPKQ | |
| | | GYVFYDTLFGDGKMPTGTGLVPFWLQSRWYPRAEFQQ | |
| | | QVLHDLYLTGPFSYKDDLKSFSFNAKYKFSFLWGGNMIP | |
| | | QQIIKNPCKKEESTFTYPSREPRDLQVVDPLTMGPEWVF | |
| | | HTANDWRRGLFGKNAVDRVSKKPDDDAEYYPVPKRPRF | |
| | | FPPTDTQSEPEKDFGFTPESQELQQEDLRAPQEESQEV | |
| | | QQQRLLQLRLSQQFRLRQQLQHLFVQVLKTQAGLHINPL | |
| | | FLNHA* | |
| AB060592.1 | BAB69900.1 | MAWIWWWQRRRRRWPWRRRRWRRLRTRRPRRLVRR | 255 |
| | | RRKRYRVRRRRRWGRRRGRRTYLRRRLKKRKRRKKLR | |
| | | LTQWNPSTIRGCTIKGMAPLIICGHTMAGNNFAIRMEDYV | |
| | | SQIRPFGGSFSTTTWSLKVLWDEHTRFHNTWSYPNTQL | |
| | | DLARFKGVNFYFYRDKDTDFIVTYSSVPPFKMDKYSSAM | |
| | | LHPGTLMQRKKKILIPSFTTRPRGRKKVKLHIKPPVLFEDK | |
| | | WYTQQDLCDVNLLSLAVSAASFRHPFCPPQTDNICITFQV | |
| | | LKDFYYTQMSVTPDTAGQEKDIEIFEKHLFKNPQFYQTVH | |
| | | TQGIISKTRRTAKFSTSNNTLGSDTNITPYLEQPTATNHKN | |
| | | TLSTGNNSIYGLPSYNPIPDKLKKIQEWFWKQETDKENLV | |
| | | TGSYQTPTNKSVSYHLGKYSPIFLSSYRTNLQFITAYTDV | |
| | | TYNPLNDKGKGNQIWVQYVTKPDTIFNERQCKCHIVDIPL | |
| | | WAAFHGYIDFIQSELGIQEEILNIAIIVVICPYTKPKLVHDPP | |
| | | NQNQGFVFYDTQFGDGKMPEGSGLVPIYYQNRWYPRIK | |
| | | FQSQVVHDFILTGPFSYKDDLKSTVLTVEYKFKFLWGGN | |
| | | MIPEQVIRNPCKTEGHDLPHTSRLHRDLQVVDPHTVGPQ | |
| | | WALHTWDWRRGLFGSEAIKRVSEQQVHDELYYPASKKP | |
| | | RFLPPISGLQEQERDYSSQEEKDQSSSEEEKDPKKKEQK | |
| | | QQQRLHLQFQEQQRLGNQLRLIFRELQKTQAGLHINPML | |
| | | SNRL* | |
| AB060593.1 | BAB69904.1 | MAWRWWWRRRWKPRRRPAWTKYRRRRWRRLRPRRP | 256 |
| | | RRLARGRRRRRTVRRRRVRRLRRRRGWTRRRYLRRRK | |
| | | RRKLILTQWNPNIVRRCSIKGIIPLTMCGANTASFNYGMH | |
| | | SDDSTPQPEKFGGGMSTVTFSLYVLYDQFTRHMNRWSY | |
| | | SNDQLDLARYRGCSFKLYRNPTTDFIVQYDNNPPMKNTIL | |
| | | SSPNTHPGMLMQQKHRILVPSWQTFPRGRKYVKVKIPPP | |
| | | KLFEDHWYTQPDLCKVPLVTLRSTAADFRHPFCSPQTNN | |
| | | PCTTFQVLRENYNEVLGLPYANTGSNNEVKIKIDNFENWL | |
| | | YNSSVHYQTFQTEQMFRPKQYNADGSTWKDYKSMLST | |
| | | WTSQIYNKKTDSNYGYASYDFSKGKEFATQMRQHYWVQ | |
| | | LTQLTATVPHIGPTYSNTTTPEYEYHAGWYSPVFIGPNRH | |
| | | NIQFRTAYMDVTYNPLNDKGQFNRVWFQYSTKPTTDFN | |
| | | NTQCKCVLENIPLWSALFGYSEYVESQLGPFQDHGTVGV | |
| | | VVVQCPYTVPPMYNKEKPDMGYVFYDTHFGNGKLGNGS | |
| | | GQVPRYWQMRWYPILKRQKQVMNDICKTGPFSYRDELL | |
| | | QVDLASPYTFRFNWGGDLLYHQVIKDPCSSSGLAPTDSS | |
| | | RFKRDVQVVSPLTMGPRLLFHSFDQRRGFFTPGAIKRMH | |
| | | DEQINVPDFTQKPKIPRIFPPVELRERAEAEEDSGSEKAS | |
| | | FTSSQEREAEAQEKLPIQLQLRQQLRQQQQLRVHLQQVF | |
| | | LQLQKTKAHLHINPLFLAQGNM* | |
| AB060595.1 | BAB69912.1 | MAYSYWWRRRRWPWRGRWRRWRRRRRIPRRRPRRP | 257 |
| | | VRRYRRRPVRRKRRWGRRGRRRRYTRRYRRRLTVRRK | |
| | | RNKLRLSVWQPQNIRYCAIKGLFPILICGHGKSAGNYAIHS | |
| | | DDFITSRFSFGGGLSTTSYSLKLLFDQNLRGLNRWTASN | |
| | | DQLDLARYLGAIFWFYRDQKTDYIVQYDISEPFKIDKDSS | |
| | | PSFHPGILMKSKHKVLVPSFQTWPKGRSKVKLKIKPPKM | |
| | | FVDKWYTQEDLCTVTLVSLVVSLASFQHPFCRPLTDNPC | |
| | | VTFQVLQNFYNNVIGYSSSDTLVDNVFTSLLYSKASFWQ | |
| | | SHLTPSYVKKINNNPDGSSISQRVGTMPDMTEYNKWVSN | |
| | | TNIGTGFVNSNVSVHYNYCQYNPNHTHLTTLRQYYFFWE | |
| | | THPAAANKTPVTHVPITTTKPTKDWWEYRLGLFSPIFLSP | |
| | | LRSSNIEWPFAYRDIIYNPLMDKGVGNMMWYQYNTKPDT | |
| | | QFSPTSCRAVLEDKPIWSMAYGYADFLLSILGEHDDVDF | |
| | | HGLVCIICPYTRPPLFDKDNPKMGYVFYDAKFGNGKWID | |
| | | GTGFIPVEFQSRWKPELAFRKDVLTDLAMSGPFSYSDDL | |
| | | KNTTIQAKYKFKFKWGGNLSYHQTIRNPCTSDGQTPTTS | |
| | | RQSREVQIVDPLTMGPRYVFHSWDWRRGWLNDRTLKR | |
| | | LFQKPLDFEEYPKSPKRPRIFPPTEQLQEDPQEQERDSS | |
| | | SSEESLPTSSEETPPAHLLRVHLRKQLRQQRDLRVQLRA | |
| | | LFAQVLKTQAGLHINPLLLAPQ* | |
| AB064596.1 | BAB79314.1 | TAWWWGRRWRRRPWGRWRRRRRVWRRRPRTAVRRR | 258 |
| | | RGRRYVSRRRRYRRRLRRRGRRRYRGRRKKRQTLVLK | |
| | | QWQPDVNRLCRITGWLPLIVCGTGRAQDNFIVHSEDITP | |
| | | RGAAYGGNLTHITWCLEAIYQEFLMHRNRWSRSNHDLDL | |
| | | CRYQGVVFKAYRHPKVDYILAYTRTPPFQATELSYMSCH | |
| | | PLLMLTAKHRIVVKSQETKKGGKKYVKFRIKPPRLMLNKW | |
| | | YFTHDFCKVPLFSMWASACDLRNPWLREGALSPTVGFF | |
| | | ALKPDFYPNLSILPNEVSQQFDFFLNSAHPPSIQSEKDVR | |
| | | WEYTYTNLMRPIYNQTPSLKASTYDWQNYSNPNNYQAC | |
| | | HQQFIAFKAQRFAKIKAEYQTVYPTLTTQTPQSEALTQEF | |
| | | GLYSPYYLTPTRISLDWHTVFHHIRYNPMADKGLGNMIW | |
| | | VDWCSRKEATYDPTRSKCMLKDLPLYMRFYGYCDWVTK | |
| | | SIGSETAWRDMRLMVVCPYTEPQLMKKNDKTWGYVIYG | |
| | | YNFANGNMPWLQPYIPISWFCRWFPCITHQREAMESVV | |
| | | ATGPFMVRDQDRNSWDITIGYKFLWRWGGSPLPTQAID | |
| | | DPCQQGTHPLPEPGTLPRILQVSDPTQLGPKTIFHLWDQ | |
| | | RRGLFSKRSIERMSEYKGTDDLFSPGRPKRPKLDTRPEG | |
| | | LPEEQRGAYNLLQALEDSAQSEESDQEEMPPLEEEQVL | |
| | | HEQKKEALLQQLQQQKHHQRVLKRGLRLLLGDVLKLRR | |
| | | GLHIDPVLT* | |
| AB064597.1 | BAB79318.1 | TAWWWGRWRRRWRRRRPWRPRLRRRRARRAFPRRR259 | |
| | | RRRFVSRRWRRPYRRRRRRGRRRRRRRRRHKPTLVLR | |
| | | QWQPDVIRHCKITGRMPLIICGKGSTQFNYITHADDITPR | |
| | | GASYGGNFTNMTFSLEAIYEQFLYHRNRWSASNHDLELC | |
| | | RYKGTTLKLYRHPDVDYIVTYSRTGPFEISHMTYLSTHPL | |
| | | LMLLNKHHIVVPSLKTKPRGRKAIKVRIRPPKLMNNKWYF | |
| | | TRDFCNIGLFQLWATGLELRNPWLRMSTLSPCIGFNVLK | |
| | | NSIYTNLSNLPQHREDRLNIINNTLHPHDITGPNNKKWQY | |
| | | TYTKLMAPIYYSANRASTYDLLREYGLYSPYYLNPTRINLD | |
| | | WMTPYTHVRYNPLVDKGFGNRIYIQWCSEADVSYNRTK | |
| | | SKCLLQDMPLFFMCYGYIDWAIKNTGVSSLARDARICIRC | |
| | | PYTEPQLVGSTEDIGFVPITETFMRGDMPVLAPYIPLSWF | |
| | | CKWYPNIAHQKEVLEAIISCSPFMPRDQGMNGWDITIGYK | |
| | | MDFLWGGSPLPSQPIDDPCQQGTHPIPDPDKHPRLLQVS | |
| | | NPKLLGPRTVFHKWDIRRGQFSKRSIKRVSEYSSDDESL | |
| | | APGLPSKRNKLDSAFRGENPEQKECYSLLKALEEEETPE | |
| | | EEEPAPQEKAQKEELLHQLQLQRRHQRVLRRGLKLVFTD | |
| | | ILRLRQGVHWNPELT* | |
| AB064599.1 | BAB79326.1 | TAWWRYRRRPWRRWRRRRWGLRTRRPRRTFRRRRAR | 260 |
| | | RYVSRGRRRRYRRRRRRGRRRRGRRRRHRKTLIVRQW | |
| | | QPDVIKRCFITGWLPLIICGNGHTQFNFITHMDDIPPKNAS | |
| | | YGGNFTNLTFNLACFYDEFMHHRNRWSASNHDLELVRYI | |
| | | RTSLKLYRHESVDYIVCYTTTGPFETNEMSYMLTHPLAML | |
| | | LSKRHVVVPSLKTKPHGRKYKKITIKPPKLMLNKWYFATD | |
| | | LCHIGLFQLWATGLELRNPWLRSGTNSPVIGFYVLKNQV | |
| | | YKNRYSNLNTTEAHNARQDAWNELTQTKTNDKWYNWQ | |
| | | YTYNKLMKPIYYAASNESSNSAMKGKTYNWKHYKEYFSN | |
| | | TQTKWKTIIKDAYDLVREEYQQLYTTTMAYPPPWQSTTS | |
| | | NTGRQYLEHDCGIYSPYFLTPQIYSPEWHTAWSYIRYNPL | |
| | | TDKGIGNRVCVQYCSEASSDYNPIKSKCMLQDMPLWMM | |
| | | LYGYADYVVKSTGIQSAWTDMRVAIRCPYTDPKLVGSTE | |
| | | NTMFIPIGLEFMNGDIPDKRPYIPLTWWFKWYPMITHQKT | |
| | | AIEAIVSCSPFMPRDQEQASWDITVGYKATFLWGGSPLP | |
| | | PQPIDDPCQKGKHDIPDPDTNPPRIQISDPQHLGPATLFH | |
| | | SWDLRRGYINTKSIKRISEHLDANEYFSTGVVSKKPRFDT | |
| | | PHHGQLSNQEEDALSILRQPQKEQEETTSEEEQALQKEE | |
| | | EQKEKLLQQLRVQRQHQRVLRQGIKHLMGDVLRLRQGV | |
| | | HWNPVL* | |
| AB064600.1 | BAB79330.1 | TAWGWYRRRRWRPWRRRRWAIRRRRPRRTVRRRGRR | 261 |
| | | RYVSRWPRRRYRRRRRRTRRRGGRKRRHRQTLILRQW | |
| | | QPDVMKKCFITGWMPLIICGTGNTQFNFITHEDDVPPKGA | |
| | | SYGGNLTNLTFTLEGLYDEHLLHRNRWSRSNFDLDLSRY | |
| | | LYTIIKLYRHESVDYIVTYNRTGPFEISPLSYMNTHPMLML | |
| | | LNKHHVVVPSPKTKPKGKRAIKIKIKPPKLMLNKWYFARD | |
| | | TCRIGLFQLYATGANLTNPWLRSGTNSPVVGFYVIKNSIY | |
| | | QDAFDNLADTEHTNQRKNVFENKLYPTTTTNKDNWQYT | |
| | | YTSLMKNIYFKTKQEAENQTMSSTYNFDTYKTNYDKVRT | |
| | | KWIKIAEDGYKLVSKEYKEIYISTATYPPQWNSRNYLSHD | |
| | | YGIYSPYFLTPQRYSPQWHTAWTYVRYNPLTDKGIGNRI | |
| | | FVQWCSEKNSSYNSTKSKCMLQDMPLFMLTYGYLDYVL | |
| | | KCAGSKSAWTDMRVCIRSPYTEPQLTGNTDDISFVIISEA | |
| | | FMNGDMPYLAPHIPVSLWFKWYPMILHQKAALETIVSCG | |
| | | PFMPRDQEANSWDITAGYKAVFKWGGSPLPPQPIDDPY | |
| | | QKPTHEIPDPDKHPPRLQIADPKILGPSTVFHTWDIRRGL | |
| | | FSTASLKRVSEYQPPDDLFSTGVASKRPRFDTPVQGQLE | |
| | | SQEEESYRLLRALQKEQETSSSEEEQPQNQEIQEKLLLQ | |
| | | LQQQRQQQRLLAKGIKHLLGDVLRLRKGVHWDPVLT* | |
| AB064601.1 | BAB79334.1 | TAWYRRRRWRPWRRRRRPWTLRRRRARRFVRRRPRR | 262 |
| | | RYVSRWRRRRYRRRLRRGRRRRGRRRRKETIIVRQWQ | |
| | | PDVMRNCYITGFLPLIVCGSGNTQFNFITHENDIPPRGAS | |
| | | YGGNLTNITFTLAALYDQYLLHRNRWSRSNFDLDLARYIN | |
| | | TKLKLYRHDSVDYIVTYNRTGPFEVNPLTYMHTHPLLMLV | |
| | | NRHHIVVPSLKTKPRGKRYIKVKIKPPKLMLNKWYFAKDIC | |
| | | PLGLFQLYATGLELRNPWIREGTNSPIVGFYVLKPSLYNG | |
| | | AMSNLADTEHLNQRQTLFNKLLPTQNQKDEWQYTYNKP | |
| | | MQKIYYEAANKQDSGFKNTTYNWTNYKTNYQKVQSQW | |
| | | QTVAQQNYNQVYNEFKEVYPLTATWPPQWNARQYMSH | |
| | | DFGIYSPYFLSPARFTDYWHSAYTYVRYNPMSDKGIGNII | |
| | | CIQWCSEKNSEFNETKNKCILRDMPLYMLTYGYLDYTTK | |
| | | CTGSNSIWTDARVAIRCPYTDPPLSNPTNKNTLYIPLSTSF | |
| | | MQGDMPWPTTNIPLKMWFKWYPMIMHQRACLETIVSCG | |
| | | PFMPRDQTASSWDITIAYRAFFKWGGNPLPPQPIDDPCQ | |
| | | KDTHEIPDPDKHPRGIQISDPKVLGPPTVFHTWDIRRGLF | |
| | | SSTSLKRVSEYQPPDDPFSTGVVFKRPRLETQYKGTQET | |
| | | PEEDAYTLLKALQKEQESSSSEEELPQEEQEIQKTQLLKQ | |
| | | LQLQQQQQRILKRGIRHLFGDVLRLRKGVHSNPDLL* | |
| AB064602.1 | BAB79338.1 | TAWYRYRRRPWRRRRRPRWGLRRRRFRRSFRGRGRR | 263 |
| | | RYVSRWSRRRYRRRRRRGRRRRGRRRRKRQTLIPRQW | |
| | | QPDVTKKCFITGWMPLIICGTGHTQFNFITHEEDIPGAGA | |
| | | SYGGNLTNITITLGGLYEQYMLHRNHWSRSNYDLELARY | |
| | | LGFTLKCYRHATVDYILTYSRTTPFETNELSHMLTHPLLM | |
| | | LLNKHHRVIPSLKTRPKGKRSVRIHIKPPKLMINKWYFAKD | |
| | | LCNIGPCQIYATGLELSNPWLRSGTNSPVIGFWVLKNHLY | |
| | | DGNLSNIASGEQLTARQTLFTTKLLPSNNTKDEWQYAYT | |
| | | PLMKTFYTQAANTAAHNITDKTYNWKNYKTHYDKVQQT | |
| | | WTTKAQFNYDLVKEEYKTVYPTTATFPPEWSNRQYLEH | |
| | | DYGLFSPYFLTPNRYSTEWHMPITYVRYNPLADKGIGNRI | |
| | | YMQWCSESSSSFEPTKSKCMLQDMPLYMLTYGYLDYVV | |
| | | KCTGVKSAWTDMRVAIRSPYTFPQLIGSTDKVGFIPLGEK | |
| | | FMSGDTDPVKNFIPLKYWYRWYPFAANQKSVLETIVSCG | |
| | | PFMPRDQEAGSWDITVGYKATFKRGGSPLPPQPIDDPC | |
| | | QKPTHDLPDPDRHPPRIQISDPARLGPETLFHSWDIRRG | |
| | | YINTKAIKRISDYTESNDYFSTGVVSKRPRLETQYHGQHE | |
| | | SQEEDAYLLLKQLQEEQETSSSEGEQAPQEKTLQKEKLL | |
| | | KQLQLHKQQQQLLRKGIRHLLGDVLRLRRGVHWDPGL* | |
| AB064603.1 | BAB79342.1 | TAWWWGRWRQRRWGRRRRRPWRVRRRRPRRSFRRR | 264 |
| | | RRGRYVSRRRRRRYYRRRLRRGRRRGRRKRHRPTLILR | |
| | | QWQPDVVKHCKITGWMPLIICGSGSTQMNFITHMDDTPP | |
| | | MGYTYGGNFVNVTFSLEAIYEQFLYHRNRWSRSNHDLDL | |
| | | ARYQGTTLKLYRHATVDYILSYNRTGPFQISEMTYMSTHP | |
| | | AIMLLMKHRIVVPSLRTKPKGRRSIKIRIKPPKLMLNKWYF | |
| | | TKDICSMGLFQLMATGAELTNPWLRDTTKSPVIGFRVLKN | |
| | | SVYTNLSNLKDVSISGERKSILNKIHPETLTGSGNASKGW | |
| | | EYSYTKLMAPIYYSAVRNSTYNWQNYQTHCATTAIKFKE | |
| | | KQTSTLTLIKAEYLYHYPNNVTQVDFIDDPTLTHDFGIYSP | |
| | | YWITPTRISLDWDTPWTYVRYNPLSDKGIGNRIYAQWCS | |
| | | EKSSKLDTTKSKCILKDFPLWCMAYGYCDWVVKCTGVSS | |
| | | AWTDMRVAIICPYTEPALIGSDENVGFIPVSDTFCNGDMP | |
| | | FLAPYIPITWWIKWYPMITHQKEVLEAIVNCGPFVPRDQS | |
| | | SPAWEITMGYKMDWKWGGSPLPSQAIDDPCQKPTHELP | |
| | | DPDRHPRMLQVSDPTKLGPKTVFHKWDWRRGQLSKRSI | |
| | | KRVQEDSTDDEYVTGPLSRKRNKLDTKMPGPPTPEKES | |
| | | YTLLQALQESGQESSSQDEEQAPQKEENQKEALVEQLQ | |
| | | LQKQHQRVLKRGLKLLLGDVLRLRRGVHWDPLLS* | |
| AB064604.1 | BAB79346.1 | MAWGWWKRKRRWWWRKRWTRGRLRRRWPRRSRRR | 265 |
| | | PRRRRVRRRRRWRRGRPRRRLYRRGRRYRRKRKRAKI | |
| | | TIRQWQPAMTRRCFIRGHMPALICGWGAYASNYTSHLED | |
| | | KIVKGPYGGGHATFRFSLQVLCEEHLKHHNYWTRSNQD | |
| | | LELALYYGATIKFYRSPDTDFIVTYQRKSPLGGNILTAPSL | |
| | | HPAEAMLSKNKILIPSLQTKPKGKKTVKVNIPPPTLFVHKW | |
| | | YFQKDICDLTLFNLNVVAADLRFPFCSPQTDNVCITFQVL | |
| | | AAEYNNFLSTTLGTTNESTFIENFLKVAFPDDKPRHSNILN | |
| | | TFRTEGCMSHPQLQKFKPPNTGPGENKYFFTPDGLWGD | |
| | | PIYIYNNGVQQQTAQQIREKIKKNMENYYAKIVEENTIITKG | |
| | | SKAHCHLTGIFSPPFLNIGRVAREFPGLYTDVVYNPWTDK | |
| | | GKGNKIWLDSLTKSDNIYDPRQSILLMADMPLYIMLNGYID | |
| | | WAKKERNNWGLATQYRLLLTCPYTFPRLYVETNPNYGY | |
| | | VPYSESFGAGQMPDKNPYVPITWRGKWYPHILHQEAVIN | |
| | | DIVISGPFTPKDTKPVMQLNMKYSFRFTWGGNPISTQIVK | |
| | | DPCTQPTFEIPGGGNIPRRIQVINPKVLGPSYSFRSFDLR | |
| | | RDMFSGSSLKRVSEQQETSEFLFSGGKRPRIDLPKYVPP | |
| | | EEDFNIQERQQREQRPWTSESESEAEAQEETQAGSVRE | |
| | | QLQQQLQEQFQLRRGLKCLFEQLVRTQQGVHVDPCLV* | |
| AB064606.1 | BAB79354.1 | MAWGWWKRRRRWWFRKRWTRGRLRRRWPRSARRRP | 266 |
| | | RRRRVRRRRRWRRGRPRRRLYRRYRRKKRRRRKPKTV | |
| | | LKQWQPDITKRCYIIGYIPAIICGAGTWSHNYTSHLLDIIPK | |
| | | GPFGGGHSTMRFSLKVLFEEHLRHLNFWTRSNQDLELV | |
| | | RYFRCSFRFYRDQHTDYLVHYSRKTPLGGNRLTAPSLHP | |
| | | GVQMLSKNKIIVPSYDTKPKGKSYVKVTIAPPTLLTDKWY | |
| | | FSKDICDTTLVNLDVVLCNLRFPFCSPQTDNPCITFSVLHS | |
| | | IYNDFLSIVDTGNYKTQFVSNLSTKVGTDWGKRLNTFRTE | |
| | | GCYSHPKLPKKAVTPGNDKTYFTVPDGLWGDAVFNAEA | |
| | | SNIITKNMESYSESAKARGVQGDPAFCHLTGIYSPPWLTP | |
| | | GRISPETPGLYTDVTYNPYADKGVGNRIWVDYCSKKGNK | |
| | | YDNTSKCLLEDMPLWMVTFGYVDWVKKETGNWGIPLWA | |
| | | RVLIRCPYTVPKLYNEADPNYGWVPYSYYFGEGKMPNG | |
| | | DLYVPFKIRMKWYPSMWNQEPVLNDLAKSGPFAYKDTK | |
| | | TSVTVTAKYKFTFNFGGNPVPSQIVQDPCTQSTYDIPGTG | |
| | | NLPRRIQVIDPKVLGPHYSFHRWDFRRGLFGQQAIKRVS | |
| | | EQPTTSEFLFSGPKRPRIDQGPYIPPEKGSDSLQRESRP | |
| | | WSNSETEAETEAPSEEEPENQEEQVLQLQLRQQLREQR | |
| | | KLRQGIQCLFEQLITTQQGVHKNPLLE* | |
| DQ186994.1 | ABD34286.1 | MAWSWWWRRRKRWWPRRRRRWRRFRTRRARRAVPR | 267 |
| | | RRRRRRVRRRRWGRRRRRRRVFYKRRRRKTGRLYRKP | |
| | | KKKLVLTQWHPTTVRNCSIRGLVPLVLCGHTQGGRNFAL | |
| | | RSDDYPKQGSPYGGSFSTTTWNLRVLFDEHQKHHNTW | |
| | | SYPNNQLDLGRYKGCTFYFYRDKKTDYIVKFQRRGPFKI | |
| | | NKYSSPMAHPGMMMLDKMKILVPSFDTRPGGRRRVKVT | |
| | | IRPPTLLEDKWYTQQDLAPVNLVSLVVSAASFIHPFSQPQ | |
| | | TNNICTTFQVLKDMYYDCIGINSTLTTKYENLFNKLYSKCC | |
| | | YFETFQTIAQLNPGFKAAKKTTNGSGSTAATLGDAVTELK | |
| | | NPNGTFYTGNNSTFGCCTYKPTKEIGSNANKWFWHQLT | |
| | | ATDSDTLGQYGRASIKYMEYHTGIYSSIFLSPLRSNLEFPT | |
| | | AYQDVTYNPLTDRGIGNRIWYQYSTKENTTFNETQCKCV | |
| | | LSDLPLWSMFYGYVDFIESELGISAEIHNFGIVCVQCPYTF | |
| | | PPMFDKSKPDKGYVFYDTLFGNGKMPDGSGHVPTYWQ | |
| | | QRWWPRFSFQRQVMHDIILTGPFSYKDDSVMTGITAGYK | |
| | | FKFSWGGDMVSEQVIKNPERGDGRDSTYPDRQRRDLQ | |
| | | VVDPRSMGPQWVFHTFDYRRGLFGKDAIKRVSEKPTDP | |
| | | DYFTTPYKKPRFFPPTAGEEKLQEEDSALQEKRSPLSSE | |
| | | EGQTRAQVLQQQVLQSELQQQQELGEQLRFLLREMFKT | |
| | | QAGIHMNPRAFQEL* | |
| DQ186995.1 | ABD34288.1 | MAWSWWWRRRKRWWPRRRRRWRRFRTRRARRAVPR | 268 |
| | | RRRRRRVRRRRWGRRRRRRRVFYKRRRRKTGRLYRKP | |
| | | KKKLVLTQWHPTTVRNCSIRGLVPLVLCGHTQGGRNFAL | |
| | | RSDDYPKQGSPYGGSFSTTTWNLRVLFDEHQKHHNTW | |
| | | SYPNNQLDLGRYKGCTFYFYRDKKTDYIVKFQRRGPFKI | |
| | | NKYSSPMAHPGMMMLDKMKILVPSFDTRPGGRRRVKVT | |
| | | IRPPTLLEDKWYTQQDLAPVNLVSLVVSAASFIHPFSQPQ | |
| | | TNNICTTFQVLKDMYYDCIGINSTLTTKYENLFNKLYSKCC | |
| | | YFETFQTIAQLNPGFKAAKKTTNGSGSTAATLGDAVTELK | |
| | | NPNGTFYTGNNSTFGCCTYKPTKEIGSNANKWFWHQLT | |
| | | ATDSDTLGQYGRASIKYMEYHTGIYSSIFLSPLRSNLEFPT | |
| | | AYQDVTYNPLTDRGIGNRIWYQYSTKENTTFNETQCKCV | |
| | | LSDLPLWSMFYGYVDFIESELGISAEIHNFGIVCVQCPYTF | |
| | | PPMFDKSKPDKGYVFYDTLFGNGKMPDGSGHVPTYWQ | |
| | | QRWWPRFSFQRQVMHDIILTGPFSYKDDSVMTGITAGYK | |
| | | FKFSWGGDMVSEQVIKNPERGDGRDSTYPDRQRRDLQ | |
| | | VVDPRSMGPQWVFHTFDYRRGLFGKDAIKRVSEKPTDP | |
| | | DYFTTPYKKPRFFPPTAGEEKLQEEDSALQEKRSPLSSE | |
| | | EGQTRAQVLQQQVLQSELQQQQELGEQLRFLLREMFKT | |
| | | QAGIHMNPRAFQEL* | |
| DQ186996.1 | ABD34290.1 | MAWGWWRWRRRWPARRWRRRRRRRPVRRTRARRPA | 269 |
| | | RRYRRRRTVRTRRRRWGRRRYRRGWRRRTYVRKGRH | |
| | | RKKKKRLILRQWQPATRRRCTITGYLPIVFCGHTKGNKNY | |
| | | ALHSDDYTPQGQPFGGALSTTSFSLKVLFDQHQRGLNK | |
| | | WSFPNDQLDLARYRGCKFYFYRTKQTDWIGQYDISEPYK | |
| | | LDKYSCPNYHPGNMIKAKHKFLIPSYDTNPRGRQKIIVKIP | |
| | | PPDLFVDKWYTQEDLCSVNLVSLAVSAASFLHPFGSPQT | |
| | | DNPCYTFQVLKEFYYQAIGFSATDQQREKVFDILYKNNSY | |
| | | WESNITPFYVINVKKGSNTTQYMSPQISDSSFRKKVNTNY | |
| | | NWYTYDAKTNASQLKQLRNAYFKQLTSEGPQHTYSDNG | |
| | | YASQWTTPSTDAYEYHLGMFSTIFLAPDRPVPRFPCAYQ | |
| | | DVTYNPLMDKGVGNHVWFQYNTKADTQLIVTGGSCKAHI | |
| | | QDIPLWAAFYGYSDFIESELGPFVDADTVGLICVICPYTKP | |
| | | PMYNKTNPMMGYVFYDRNFGDGKWTDGRGKIEPYWQV | |
| | | RWRPEMLFQETVMADIVQTGPFSYKDELKNSTLVCKYKF | |
| | | YFTWGGNMMFQQTIKNPCKTDGQPTDSSRHPRGIQVAD | |
| | | PEQMGPRWVFHSFDWRRGYLSEKALKRLQEKPLDYDEY | |
| | | FTQPKRPRIFPPTESAEGEFREPEKGSYSEEERSQASAE | |
| | | EQTEEATVLLLKRRLREQQQLQQQLQFLTREMFKTQAGL | |
| | | HINPMLLNQR* | |
| DQ186997.1 | ABD34292.1 | MAWGWWRWRRRWPARRWRRRRRRRPVRRTRARRPA | 270 |
| | | RRYRRRRTVRTRRRRWGRRRYRRGWRRRTYVRKGRH | |
| | | RKKKKRLILRQWQPATRRRCTITGYLPIVFCGHTKGNKNY | |
| | | ALHSDDYTPQGQPFGGALSTTSFSLKVLFDQHQRGLNK | |
| | | WSFPNDQLDLARYRGCKFYFYRTKQTDWIGQYDISEPYK | |
| | | LDKYSCPNYHPGNMIKAKHKFLIPSYDTNPRGRQKIIVKIP | |
| | | PPDLFVDKWYTQEDLCSVNLVSLAVSAASFLHPFGSPQT | |
| | | DNPCYTFQVLKEFYYQAIGFSATDEQREKVFDILYKNNSY | |
| | | WESNITPFYVINVKKGCNTTQYMSPQISDSSFRKKVNTNY | |
| | | NWYTYDAKTNASQLKQLRNAYFKQLTSEGPQHTYSDNG | |
| | | YASQWTTPSTDAYEYHLGMFSTIFLAPDRPVPRFPCAYQ | |
| | | DVTYNPLMDKGVGNHVWFQYNTKADTQLIVTGGSCKAHI | |
| | | QDIPLWAAFYGYSDFIESELGPFVDADTVGLICVICPYTKP | |
| | | PMYNKTNPMMGYVFYDRNFGDGKWTDGRGKIEPYWQV | |
| | | RWRPEMLFQETVMADIVQTGPFSYKDELKNSTLVCKYKF | |
| | | YFTWGGNMMFQQTIKNPCKTDGQPTDSSRHPRGIQVAD | |
| | | PEQMGPRWVFHSFDWRRGYLSEKALKRLQEKPLDYDQ | |
| | | YFTQPKRPRIFPPTESAEGEFREPEKGSYSEEERLQASA | |
| | | EEQTEEATVLLLKRRLREQQQLQQQLQFLTREMFKTQAG | |
| | | LHINPMLLNQR* | |
| DQ186998.1 | ABD34294.1 | MAWGWWRWRRRWPARRWRRRRRRRPVRRTRARRPA | 271 |
| | | RRYRRRRTVRTRRRRWGRRRYRRGWRRRTYVRKGRH | |
| | | RKKKKRLILRQWQPATRRRCTITGYLPIVFCGHTKGNKNY | |
| | | ALHSDDYTPQGQPFGGALSTTSFSLKVLFDQHQRGLNK | |
| | | WSFPNDQLDLARYRGCKFYFYRTKQTDWIGQYDISEPYK | |
| | | LDKYSCPNYHPGNMIKAKHKFLIPSYDTNPRGRQKIIVKIP | |
| | | PPDLFVDKWYTQEDLCSVNLVSLAVSAASFLHPFGSPQT | |
| | | DNPCYTFQVLKEFYYQAIGFSATDEQREKVFDILYKNNSY | |
| | | WESNITPFYVINVKKGCNTTQCMSPQISDSSFRKKVNTN | |
| | | YNWYTYDAKTNASQLKQLRNAYFKQLTSEGPQHTYSDN | |
| | | GYASQWTTPSTDAYEYHLGMFSTIFLAPDRPVPRFPCAY | |
| | | QDVTYNPLMDKGVGNHVWFQYNTKADTQLIVTGGSCKA | |
| | | HIQDIPLWAAFYGYSDFIESELGPFVDADTVGLICVICPYT | |
| | | KPPMYNKTNPMMGYVFYDRNFGDGKWTDGRGKIEPYW | |
| | | QVRWRPEMLFQETVMADIVQTGPFSYKDELKNSTLVCKY | |
| | | KFYFTWGGNMMFQQTIKNPCKTDGQPTDSSRHPRGIQV | |
| | | ADPEQMGPRWVFHSFDWRRGYLSEKALKRLQEKPLDY | |
| | | DQYFTQPKRPRIFPPTESAEGEFREPEKGSYSEEERSQA | |
| | | SAEERTEEATVLLLKRRLREQQQLQQQLQFLTREMFKTQ | |
| | | AGLHINPMLLNQR* | |
| DQ186999.1 | ABD34296.1 | MAWRWWKRRRRWWFRKRWTRGRLRRRWPRPARRRP | 272 |
| | | RRRRVRRRRRWRRGRPRRRLYRRYRRKKRRRRKPKIIL | |
| | | KQWQPDIVKRCYIVGYIPAIICGAGTWSHNYTSHLLDIIPK | |
| | | GPFGGGHSTMRFSLKVLSEEHLRHLNFWTKSNQDLELIR | |
| | | YFRCSFKFYRDQDTDYIVHYSRKTPLGGNRLTAPNLHPG | |
| | | VQMLSKNKIIVPSYATKPKGPSYIKVTIAPPTLLTDKWYFS | |
| | | KDVCDTTLVNLDVVLCNLRFPFCSPQTDNPCITFQVLHSI | |
| | | YNDFLSIVDTNNYKESFVSALPTKVSTDWGKRLNTFRTE | |
| | | GCYSHPKLHKKAVTAATDTEYFTKPDGLWGDTIFDVENG | |
| | | QKIIKNMESYAKSAKERGINGDPAFCHLTGIYSPPWLTPG | |
| | | RISPETPGLYTDVTYNPYADKGVGNRIWVDYCSKKGNKY | |
| | | DNTSKCLLEDMPLWMVCFGYVDCVKKETGNWGIPLWAR | |
| | | VLIRSPYTVPKLYNEADPNYGWVPIFYYFGEGKMPNGDM | |
| | | YIPFKIRMKWYPSMWNQEPVLNDLAKSGPFAYKNTKTSV | |
| | | TVTAKYKFTFNFGGNPVPSQIVQDPCTQPTYDIPGTGNLP | |
| | | RRIQVIDPKVLSPHYSFHRWDFRRGLFGSQAIKRVSEQS | |
| | | TTSEFLFSGPKKPRIDQGPYIPPEKGSGSLQREPRPWSS | |
| | | SETEAETEAPSEEEPENQEEQVLQLQLRQQLREQRKLR | |
| | | QGIQCLFEQLITTQQGVHKNPLLE* | |
| DQ187000.1 | ABD34298.1 | MAWRWWKRRRRWWFRKRWTRGRLRRRWPRPARRRP | 273 |
| | | RRRRVRRRRRWRRGRPRRRLYRRYRRKKHRRRKPKIIL | |
| | | KQWQPDIVKRCYIVGYIPAIICGAGTWSHNYTSHLLDIIPK | |
| | | GPFGGGHSTMRFSLKVLFEEHLRHLNFWTKSNQDLELIR | |
| | | YFRCSFKFYRDQDTDYIVHYSRKTPLGGNRLTAPNLHPG | |
| | | VQMLSKNKIMVPSYATKPKGPSYIKVTIAPPTLLTDKWYF | |
| | | SKDVCDTTLVNLDVVLCNLRFPFCSPQTDNPCITFQVLHS | |
| | | IYNDFLSIVDTNNYKESFVSALPTKVSTDWGKRLNTFRTE | |
| | | GCYSHPKLHKKAVTAATDTEYFTKPDGLWGDTIFDVENG | |
| | | QKIIKNMESYAKSAKERGINGDPAFCHLTGIYSPPWLTPG | |
| | | RISPETPGLYTDVTYNPYADKGVGNRIWVDYCSKKGNKY | |
| | | DNTSKCLLEDMPLWMVCFGYVDWVKKETGNWGIPLWA | |
| | | RVLIRSPYTVPKLYNEADPNYGWVPISYYFGEGKMPNGD | |
| | | MYIPFKIRMKWYPSMWNQEPVLNDLAKSGPFAYKNTKTS | |
| | | VTVTAKYKFTFNFGGNPVPSQIVQDPCTQPTYDIPGTGNL | |
| | | PRRIQVIDPKVLGPHYSFHRWDFRRGLFGSQAIKRVSEQ | |
| | | STTSEFLFSGPKKPRIDQGPYIPPEKGSGSLQREPRPWS | |
| | | SSETEAETEAPSEEEPENQEEQVLQLQLRQQLREQRKLR | |
| | | QGIQCLFEQLITTQQGVHKNPLLE* | |
| DQ187001.1 | ABD34300.1 | MARRWWKRRRRWWFRKRWTRGRLRRRWPRPARRRP | 274 |
| | | KRRRVRRRRRWRRGRPRRRLYRRYRRKKRRRRKPKIIL | |
| | | KQWQPDIVKRCYIVDYIPAIICGAGTWSRNYTSHLLDIIPK | |
| | | GPFGGGHSTMRFSLKVLFEEHLRHLNFWTKSNQDLELIR | |
| | | YFRCSFKFYRDQDTDHIVHYSRKTPLGGNRLTAPNLHPG | |
| | | VQMLSKNKIIVPSYATKPKGPSYIKVTIAPPTLLTDKWYFS | |
| | | KDVCDTTLVNLDVVLCNLRFPFCSPQTDNPCITFQVLHSI | |
| | | YNDFLSIVDTNNYKESFVAALPTKVSTDWGKRLNTFRTE | |
| | | GCYSHPKLHKKAVTAATDTEYFTKPDGLWGDTIFDVENG | |
| | | QKIIKNMESYAKSAKERGINGDPAFCHLTGIYSPPWLTPG | |
| | | RISPETPGLYTDVTYNPYADKGVGNRIWVDYCSKKGNKY | |
| | | GNTSKCLLEDMPLWMVCFGYVDWVKKETGNWGIPLWA | |
| | | RVLIRSPYTVPKLYNEADPNYGWVPISYYFGEGKMPNGD | |
| | | MYVPFKIRMKWYPSMWNQEPVLNDLAKSGPFAYKNTKT | |
| | | SVTVTAKYKFTFNFGGNPVPSQIVQDPCTQPTYDIPGTG | |
| | | NLPRRIQVIDPKVLGPHYSFHRWDFRRGLFGSQAIKRVS | |
| | | EQSTTSEFLFSGPKKPRIDQGPYIPPEKGSGSLQREPRP | |
| | | WSSSETEAETEAPSEEEPENQEEQVLQLQLRQQLREQR | |
| | | KLRQGIQCLFEQLITTQQGVHKNPLLE* | |
| DQ187002.1 | ABD34302.1 | MAWRWWKRRRRWWFRKRWTRGRLRRRWPRPARRRP | 275 |
| | | KRRRVRRRRRWRRERPRRRLYRRYRRKKRRRRKPKIIL | |
| | | KQWQPDIVKRCYIVGYIPAIICGAGTWSHNYTSHLLDIIPK | |
| | | GPFGGGHSTMRFSLKVLFEEHLRHLNFWTKSNQDLELIR | |
| | | YFRCSFKFYRDQDTDYIVHYSRKTPLGGNRLTAPNLHPG | |
| | | VQMLSKNKIIVPSYATKPKGPSYIKVTIAPPTLLTDKWYFS | |
| | | KDVCDTTLVNLDVVLCKLRFPFCSPQTDNPCITFQVLHSI | |
| | | YNDFLSIVDTNNYKESFVAALPTKVSTDWGKRLNTFRTE | |
| | | GCYSHPKLHKKAVTAATDTEYFTKPDGLWGDTIFDVENG | |
| | | QKIIKNMESYAKSAKERGINGDPAFCHLTGIYSPPWLTPG | |
| | | RISPETPGLYTDVTYNPYADKGVGDRIWVDYCSKKGNKY | |
| | | DNTSKCLLEDMPLWMVCFGYVDWVKKETGNWGIPLWA | |
| | | RVLIRSPYTVPKLYNEADPNYGWVPISYYFGEGKMPNGD | |
| | | MYVPFKIRMKWYPSMWNQEPVLNDLAKSGPFAYKNTKT | |
| | | SVTVTAKYKFTFNFGGNPVPSQIVQNPCTQPTYDIPGTG | |
| | | NLPRRTQVIDPKVLGPHYSFHRWDFRRGLFGSQAIKRVS | |
| | | EQSTTSEFLFSGPKKPRIDQGPYIPPEKGSGSLQREPRP | |
| | | WSSSETEAETEAPSEEEPENQEEQVLQLQLRQQLREQR | |
| | | KLRQGIQCLFEQLITTQQGVHKNPLLE* | |
| DQ187004.1 | ABD34305.1 | MAWGWWKRRRRRWWRGLWRRRRFARRRPRRPARRP | 276 |
| | | RRRRVRRRRRWRRGRLRRRVYNRRRRIRRKRRRQKLTI | |
| | | RQWQPDKRRICRIKGYLPAIIYGDGTFSKNYTSHLEDRIS | |
| | | KGPFGGGHGTARMSLKVLYDDHLKGLNIWTYSNKDLELV | |
| | | RYMHTTITFYRHPDTDFIAVYNRKTPLGGNRYTAPSLHPG | |
| | | NMMLQRTKILIPSFKTKPRGSGKIRVVIKPPTLLVDKWYFQ | |
| | | KDICDVTLFNLNITAASLRFPFCSPQTNNPCVTFQVLHSV | |
| | | YDKALGINTFGTKETPEDQQMEDIKNWLTKALNTAGFTVL | |
| | | NTFRTEGIYSHPQLKKPPEGSNKPSAEQYFAPLDSLWGD | |
| | | KIYVNNNTSPSQTEATIPGILARNACTYYQKAKTSTLRQHL | |
| | | GAMAHCHLTGIFNPALLTQGRLSPEFFGLYKEIIYNPYDD | |
| | | KGKGNRIWIDPLTKPDNIFDARSKVELEDMPLWMACFGY | |
| | | NDWCKKELNNWGLEVEYRVLLRCPYTYPKLYNDANPNY | |
| | | GYVPISYNFSAGKTVEGDLYVPIMWRTKWHPTMYNQSP | |
| | | VLEDLAMAGPFAPKEKIPSSTLTIKYKAKFIFGGNPISEQIV | |
| | | KDPCTQPTYEIPGGGTLPRRIQVINPEYIGPHYSFKSFDIR | |
| | | RGYFSAKSVKRVSEQSDITEFIFSGPKKPRIDQDRYQEAE | |
| | | EHSDSRLREEKPWESSQETESEAQEEEIQETNIQLQLQH | |
| | | QLKEQLQLRRGIQCLFEQLTKTQQGVHINPSLV* | |
| DQ187005.1 | ABD34307.1 | MSLKVLYDDHLKGLNIWTYSNKDLELVRYMHTTITFYRHP | 277 |
| | | DTDFIAVYNRKTPLGGNRYTAPSLHPGNMMLQRTKILIPS | |
| | | FKTKPRGSGKIRVVIKPPTLLVDKWYFQKDICDVTLFNLNI | |
| | | TAASLRFPFCSPQTNNPCVTFQVLHSVYDKALGINTFGTK | |
| | | ETPEDQQMEDIKNWLTKALNTAGFTVLNTFRTEGIYSHP | |
| | | QLKKPPEGSNKPSAEQYFAPLDSLWGDKIYVNNNTSPSQ | |
| | | TEATIPGILARNACTYYQKAKTSTLRQHLGAMAHCHLTGI | |
| | | FNPALLTQGRLSPEFFGLYKEIIYNPYDDKGKGNRIWIDPL | |
| | | TKPDNIFDARSKVELEDMPLWMACFGYNDWCKKELNNW | |
| | | GLEVEYRVLLRCPYTYPKLYNDANPNYGYVPISYNFSAG | |
| | | KTVEGDLYVPIMWRTKWYPTMYDQSPVLEDLAMAGPFA | |
| | | PKEKIPSSTLTIKYKAKFIFGAILYLNRLSRTPAPSPPTKFP | |
| | | EAVRSLAEYKSLTRNTSGHTTHSKASTSDVGTLARRVLK | |
| | | ECQNNQTLLSLYSQVQKSQGSTKTGTKKQKNTQILDSEK | |
| | | RNRGRARKKQRAKPKKKRYKRQTSSSSCSTSSKSNCSS | |
| | | DGESSASSSN* | |
| DQ361268.1 | ABD61942.1 | MAWRWWWRRRRPWRWRWRRRRRPARRRRRRRPAR | 278 |
| | | RARRPRVRRWRRRRVWAPRPYIRRRRRSFRRKKIKITQ | |
| | | WNPAVTKKCTVTGYLPVIYCGTGDIGTTFQNFGSHMNEY | |
| | | KQYNAAGGGFSTMLFTMQNLYEEYQKHRCRWSKSNQD | |
| | | LDLCRYLDCKLTFYRSPNTDFIVGYNRKPPFIDTQITRCTL | |
| | | HPGMLIQERKKVIIPSFQTRPKGRIKRKIKVRPPTLFTDKW | |
| | | YFQRDLCKVPLVTVSASAASLRFPFGSPQTENYCIYFQVL | |
| | | DPWYHTRLSITGGKPAEYWTQLKAYLTQGWGRSTNNAG | |
| | | YQHGPLGTYFNTLKTSEHIRQPPADNYKQANKDTTYYGR | |
| | | VDSHWGDHVYQQTIIQAMEENQSNMYTKRALHTFLGSQ | |
| | | YLNFKSGLFSSIFLDNARLSPDFKGMYQEVVYNPFNDRG | |
| | | VGNKVWVQWCTNEDTIFKDLPGRVPVVDLPLWCALMGY | |
| | | SDYCKKYFHDDGFLKEARITIISPYTNPPLINNKNTNEGFV | |
| | | PYSFYFGKGRMPDGNGYIPIDFRFNWYPCIFHQTNWIND | |
| | | MVQCGPFAYHGDEKNCSLTMKYKFKFLFGGNPISQQTIK | |
| | | DPCQQPDWQLPGSGRFPRDVQVSNPRLQTEGSTFHAW | |
| | | DFRRGFYGKRAIERLQGQQDDVTYIAGPPKRPRFEVPAL | |
| | | AAEGSSNTRRSELPWQTSEEESSQEENSEETEEETSLS | |
| | | QQLKQQCIEQKLLKRTLHQLVKQLVKTQYHLHAPIIH* | |
| EF538879.1 | ABU55887.1 | MAWRWWKRRRRWWFRKRWTRGRLRRRWPRPARRRP | 279 |
| | | RRRRVRRRRRWRRGRPRRRLYRRYRRKKRRRRKPKIIL | |
| | | KQWQPDIVKRCYIIGYIPAIICGAGTWSSHNYTSHLLDIIPKG | |
| | | PFGGGHSTMRFSLKVLFEEHLRHLNFWTKSNQDLELIRY | |
| | | FRCSFKFYRDQDTDYIVHYSRKTPLGGNRLTAPSLHPGV | |
| | | QMLSKNKILVPSYATKPKGGSYVKVTIAPPTLLTDKWYFS | |
| | | KDVCDTTLVNLDVVLCNLRFPFCSPQTDNPCITFQVLHSY | |
| | | YNDYLSIVDTALYKTSFVNNLSTKLGTRNANRLNTFRTEG | |
| | | CYSHPKLLKKTVTAANDTKYFTTPDGLWGDAVFDVSDAK | |
| | | KLTKNMESYAASANERGVQGDPAFCHLTGIFSPPWLTPG | |
| | | RISPETPGLYTDVTYNPYADKGVGNRIWVDYCSKKGNKY | |
| | | DNTSKCVLEDMPLWMLCFGYVDWVKKETGNWGIPLWA | |
| | | RVLIRSPYTVPKLYHENDPDYGWVPISYYFGEGKMPNGD | |
| | | MYVPFKVRMKWYPSMWNQEPVLNDLAKSGPFAYKNTK | |
| | | TSVTVTAKYKFTFNFGGNPVPSQIVQDPCTQPTYDIPGTG | |
| | | NLPRRIQVIDPKVLGPHYSFHRWDFRRGLFGTQAIKRVSE | |
| | | QSTTSEFLFSGPKKPRIDQGPYIPPEKGSGSLQRESRPW | |
| | | SSSETEAETEAPSEEEPENQEEQVLQLQLRQQLREQRKL | |
| | | RQGIQCLFEQLITTQQGVHKNPLLE* | |
| EU305675.1 | ABY26045.1 | MAWWGRWRRWRWRPRRWRRRRRRRVPRRRAQRSV | 280 |
| | | RRRRARRVRRRRWGRRRWRRGYRRRLRLRRKRKRKR | |
| | | RLVLTQWHPAKVRRCRISGVLPMILCGAGRSSFNYGLHS | |
| | | DDFTKQKPNNONPHGGGMSTVTFNLKVLFDQYERFMNK | |
| | | WSYPNDQLDLARYKGCKFTFYRHPEVDFLAQYDNVPPM | |
| | | KMDELTAPNTHPALLLQSRHRVKIYSWKTRPFGSKKVTV | |
| | | KIGPPKLFEDKWYSQSDLCKVSLVSWRLTACDFRFPFCS | |
| | | PQTDNPCVTFQVLGEQYYEVFGTSVLDVPASYNSQITTF | |
| | | EQWLYKKCTHYQTFATDTRLAPQKKATTSTNHTYNPSG | |
| | | NTESSTWTQSNYSKFKPGNTDSNYGYCSYKVDGETFKAI | |
| | | KNYRKQRFKWLTEYTGENHINSTFAKGKYDEYEYHLGW | |
| | | YSNIFIGNLRHNLAFRSAYIDVTYNPTVDKGKGNIVWFQY | |
| | | LTKPTTQLIRTQAKCVIEDLPLYCAFFGYEDYIQRTLGPYQ | |
| | | DIETVGVICFISPYTEPPCIRKEEQKKDWGFVFYDTNFGN | |
| | | GKTPEGIGQVHPYWMQRWRVMAQFQKETQNRIARSGP | |
| | | FSYRDDIPSATLTANYKFYFNWGGDSIFPQIIKNPCPDTGL | |
| | | RPSGHREPRSVQVVSPLTMGPEFIFHRWDWRRGFYNPK | |
| | | ALKRMLEKSDNDAESSTGPKVPRWFPAHHDQEQESDFD | |
| | | SQETRSQSSQEEAAQEALQDVQETSVQQYLLKQFREQR | |
| | | LLGQQLRLLMLQLTKTQSNLHINPRVLDHA* | |
| EU305676.1 | ABY26046.1 | MFWWGWRRRWWWKPRRRWRRRRARRPRRVPRRRY | 281 |
| | | RRAARRYRGRRVRRRRAGGWRGRRRYSRHYSRRLTVR | |
| | | RKKKKLTLKIWQPQNIRKCRIRGLLPLLICGHTRSAFNYAI | |
| | | HSDDKTPQQESFGGGLSTVSFSLKVLFDQNQRGLNRWS | |
| | | ASNDQLDLARYLGCTFWFYRDKKTDFIVQYDISAPFKLDK | |
| | | NSSPSYHPFMLMKAKHKVLIPSFDTKPKGREKIKVRIQPP | |
| | | KMFIDKWYTQEDLCPVILVSLAVSVASFTHPFCSPQTANP | |
| | | CITFQVLKEFYYPAMGYGAPETTVTSVFNTLYTTATYWQ | |
| | | SHLTPQFVRMPTKNPDNTENNQAQAFNTWVDKDFKTGK | |
| | | LVKYNFPQYAPSIEKLKQLRTYYFEWETKHTGVAAPPTW | |
| | | TTPTSDRYEYHMGMFSPTFLTPFRSAGLDFPGAYQDVTY | |
| | | NPLTDKGVGNRMWFQYNTKIDTQFDARSCKCVLEDMPL | |
| | | YAMAYGYADFLEQEIGEYQDLEANGYVCVISPYTKPPMF | |
| | | NKHNPQQGYVFYDSQWGNGKWIDGTGFVPVYWLTRWR | |
| | | VELLFQKKVLSDIAMSGPFSYPDELKNTVLTAKYRFDFKW | |
| | | GGNLFHQQTIRNPCKPEETSTGRVPRDVQVVDPVTMGP | |
| | | RFVFHSWDWRRGFLSDRALKRMFEKPLDLEGFAASPKR | |
| | | PRIFPPTEGQLAREQKEQEESSDSQEESSLTSLEEVPEE | |
| | | TKLRLHLRKQLREQRSIRQQLRTMFQQLVKTQAGLHLNP | |
| | | LLSSQL* | |
| FJ426280.1 | ACK14071.1 | MAWRWWWQRRWRRRPWPRRRWRRLRRRRPRRPVR | 282 |
| | | RRRRRATVRRRRWRGRRGRRTYTRRAVRRRRRPRKRF | |
| | | VLTQWSPQTARNCSIRGIVPMVICGHTRAGRNYALHSED | |
| | | FTTQIRPFGGSFSTERNSLKVLWDEHQKFQNRWSYPNT | |
| | | QLDLARYRGVTFWFYRDQKTDYIVQWSRNPPFKLNKYS | |
| | | SPMYHPGMMMQAKKKLVVPSFQTRPKGKKRYRVRIRPP | |
| | | NMFNDKWYTQEDLCPVPLVQIVVSAATQTKKNCSPQTN | |
| | | NPCITFQVLKDKYLNYIGVNSSETRRNSYKTLQEKLYSQC | |
| | | TYFQTTQVLAQLSPAFQPAKKPNRTNNSTSTTLGNKVTD | |
| | | LKSNNGKFHTGNNPVFGMCSYKPSKDILYKANEWLWDN | |
| | | LMVENDLHSTYGKATLKCMEYHTGIYSSIFLSPQRSLEFP | |
| | | AAYQDVTYNPNCDRAIGNRVWFQYGTKMNTNFNEQQC | |
| | | KCVLTNIPLWAAFNGYPDFIEQELGISTEVHNFGIVCFQCP | |
| | | YTFPPLYDKKNPDKGYVFYDTTFGNGKMPDGSGHIPIYW | |
| | | QQRWWIRLAFQVQVMHDFVLTGPFSYKDDLANTTLTAR | |
| | | YKFRFKWGGNIIPEQIIKNPCKREQSLGSYPDRQRRDLQV | |
| | | VDPSTMGPIYTFHTWDWRRGLFGADAIQRVSQKPEDAL | |
| | | RFTNPFKRPRYLPPTDGEDYRQEEDFALQERRRRTSTEE | |
| | | VQDEESPPQNAPLLQQQQQQRELSVQHAEQQRLGVQL | |
| | | RYILQEVLKTQAGLHLNPLLLGPPQTRCISLSPPEAYSP* | |
| FJ392105.1 | ACR20257.1 | MAAWWWGRRRRWRRWRRRRLPRRRRWRRRRRWPR | 283 |
| | | RRRRRWPRRRRRRRPARRPRRRRRRRRVRRPRRRQK | |
| | | LVLTQWNPQTVRKCIIRGFVPLFQCSRTAYHRNFVDHMD | |
| | | DVYTTGPFGGGTGSMLFTLSFFYHEFKKHHCKWSASNR | |
| | | DFDLCRYRGTVLKFYRHPDVDYIVWLNRNPPFQENLLDA | |
| | | MSRQPLIMLQTHKCILVRSFKTHPRGPSYVRMKVRPPRL | |
| | | LTDKWYFQSDFCNVPLFQLQFALAELRFPIGSPQTNTTC | |
| | | VNFLVLDNRYHLFLDNKPQQSDNSQREERGHGYPFNGS | |
| | | EGEADRLKFWHSLWNTGRFLNTTHINTLQPNISKLQEHK | |
| | | AEDTEAKTTYKSLINGNKKVYNDSQYMQNVWAQNKINTL | |
| | | YEAIAEEQYRKIQKYYNTTYGQYQRQLFTGKKYWDYRVG | |
| | | MFSPTFLSPSRLNPEMPGAYTEIAYNPWTDEGTGNVVCL | |
| | | QYLTKETSDYKPHAGSKFTIEDVPLWIAMNGYVDICKKEG | |
| | | KDPGIRLNCLMCIRCPYTRPKLYNPRYPKELFVVYSYNFA | |
| | | HGRMPGGDKYIPMEFKDRWYPSLMHQEEVIEDIVRSGPF | |
| | | ALKDQTEMVTCMMRYSALFNWGGNIIREQAVEDPCKKN | |
| | | TFALPGASGVARLLQVSNPIRQTPSTTWHSWDWRRSLF | |
| | | TQTGIKRMREQQPYDEITYAGPKRPKLTVPAGPTLAAGD | |
| | | AYNYWERKPLTSPGETLPTQTETETEAPEEEAQQEEVQE | |
| | | GLQLQQLWEQQLQQKRQLGVMFQQLLRLRTGAEIHPAL | |
| | | A* | |
| FJ392107.1 | ACR20260.1 | MAAWWWGRRRRWRRWRRRRLPRRRRWRRRRRWPR | 284 |
| | | RRRRRWPRRRRRRRPARRPRRRRRRRRVRRPRRRQK | |
| | | LVLTQWNPQTVRKCIIRGFVPLFQCSRTACHRNFVDHMD | |
| | | DVYTTGPFGGGTGSMLFTLSFFYHEFKKHHCKWSASNR | |
| | | DFDLCRYRGTVLKFYRHPDVDYIVWLNRNPPFQENLLDA | |
| | | MSRQPLIMLQTHKCILVRSFKTHPRGPSYVRMKVRPPRL | |
| | | LTDKWYFQSDFCNVPLFQLQFALAELRFPIGSPQTNTTC | |
| | | VNFLVLDNRYHLFLDNKPQQSENLQRKERGHGYSFTGN | |
| | | EGEVDRLKFWHSLWNTGRFLNTTHINTLLPNISKLQEHKA | |
| | | EDRQANAKYKNLINGNKKVYNDSQYMQNVWEENKINTL | |
| | | YDAIAEEQYRKIQKYYNTTYGQYQRQLFTGKKYWDYRVG | |
| | | MFSPTFLSPSRLNPEMPGAYTEIAYNPWTDEGTGNVVCL | |
| | | QYLTKETSDYKPHAGSKFTIEDVPLWIAMNGYVDICKKEG | |
| | | KDPGIRLNCLMCIRCPYTRPKLYNPRYPEELFVVYSYNFA | |
| | | HGRMPGGDKYIPMEFKDRWYPSLMHQEEVIEDIVRSGPF | |
| | | ALKDQTEMVTCMMRYSALFNWGGNIIREQAVEDPCKKN | |
| | | TFALPGASGVARLLQVSNPIRQTPSTTWHSWDWRRSLF | |
| | | TQTGIKRMREQQPYDEITYAGPKRPKLTVPAGPTLAAGD | |
| | | AYNYWERKPLTSPGETLPTQTDTETEAPEEEAQQEEVQ | |
| | | EGLQLQQLWEQQLQQKRQLGVMFQQLLRLRTGAEIHPA | |
| | | LA* | |
| FJ392108.1 | ACR20262.1 | MAAWWWGRRRRWRRWRRRRLPRRRRWRRRRRWPR | 285 |
| | | RRRRRWPRRRRRRRPARRPRRRRRRRRVRRPRRRQK | |
| | | LVLTQWNPQTVRKCIIRGFVPLFQCSRTAYHRNFVDHMD | |
| | | DVYTTGPFGGGTGSMLFTLSFFYHEFKKHHCKWSASNR | |
| | | DFDLCRYRGTVLKFYRHPDVDYIVWLNRNPPFQEDLLDA | |
| | | MSRQPLIMLQTHKCILVRSFKTHPRGPSYVRMKVRPPRL | |
| | | LTDKWYFQSDFCNVPLFQLQFALAELRFPIGSPQTNTTC | |
| | | VNFLVLDNRYHLFLDNKPQQSDNPQRKERGHGYSFTGN | |
| | | EGEMDRERFWHSLWSTGRFLNTTHINTLLPNISKLQDHK | |
| | | AEDKDAKTTYKSLINDNKKVYNDSQYMQNVWDQNKIHTL | |
| | | YMAIAEEQYRKIQKYYNTTYGQYQRQLFTGKKYWDYRV | |
| | | GMFSPTFLSPSRLNPEMPGAYTEIAYNPWTDEGTGNVV | |
| | | CLQYLTKETSDYKPHAGSKFTIEDVPLWIAMNGYVDICKK | |
| | | EGKDPGIRLNCLMCIRCPYTRPKLYNPRYPEELFVVYSYN | |
| | | FAHGRMPGGDKYIPMEFKDRWYPSLMHQEEVIEDIVRSS | |
| | | PFALKDQTEMVTCMMRYSALFNWGGNIIREQAVEDPCK | |
| | | KNTFALPGASGVARLLQVSNPIRQTPSTTWHSWDWRRS | |
| | | LFTQTGIKRMREQQPYDEITYAGPKRPKLTVPAGPTLAAG | |
| | | DAYNYWERKPLTSPGETLPTQTETETEAPEEEAQQEEV | |
| | | QEGLQLQQLWEQQLQQKRQLGVMFQQLLRLRTGAEIHP | |
| | | ALA* | |
| FJ392111.1 | ACR20267.1 | MAAWWWGRRRRWRRWRRRRLPRRRRWRRRRRWPR | 286 |
| | | RRRRRWPRRRRRRRPARRPRRRRRRRRVRRPRRRQK | |
| | | LVLTQWNPQTVRKCIIRGFVPLFQCSRTAYHRNFVDHMD | |
| | | DVYTTGPFGGGAGSMLFTLSFFYHEFKKHHCKWSASNR | |
| | | DFDLSRYRGAVLKFYRHPDVDYIVWLNRNPPFQENLLDA | |
| | | MSRQPLIMLQTHKCILVRSFKTHPRGPSYVRMKVRPPRL | |
| | | LTDKWYFQSDFCNVPLFQLQFALAELRFPIGSPQTNTTC | |
| | | VNFLVLDNRYHSFLDNKPQQSENSQRKERGHGYSFTGK | |
| | | EGEQDRLTFWQSLWNTGRFLNTTHINTLLPNISKLQDHK | |
| | | AEDTDANPDYKSLINGNKKVYNDSQYMQNVWQQGKINT | |
| | | LCNAIAQEQYRKIQKYYNTTYGQYQRQLFTGKKYWDYRV | |
| | | GTFSPTFLSPSRLNPEMPGAYTEIAYNPWTDEGTGNVVC | |
| | | LQYLTKETSDYKPHAGSKFTIEDVPLWIAMNGYVDICKKE | |
| | | GKDPGIRLNCLMCIRCPYTRPKLYNPRYPEELFVVYSYNF | |
| | | SHGRMPGGDKYIPMEFKDRWYPSLMHQEEVIEDIVRSG | |
| | | PFALKDQTDMVTCMMRYSALFNWGGNIIREQAVEDPCK | |
| | | KNTFALPGASGVARLLQVSNPIRQTPSTTWHSWDWRRS | |
| | | LFTQTGIKRMREQQPYDEITYAGPKRPKLTVPAGPTLAAG | |
| | | DAYNYWERKPLTSPGETLPTQTETETEAPEEEAQQEEV | |
| | | QEGLQLQQLWEQQLQQKRQLGVMFQQLLRLRTGAEIHP | |
| | | ALA* | |
| FJ392112.1 | ACR20269.1 | MAAWWWGRRRRWRRWRRRRLPRRRRWRRRRRWPR | 287 |
| | | RRRRRWPRRRRRRRPARRPRRRRRRRRVRRPRRRQK | |
| | | LVLTQWNPQTVRKCIIRGFVPLFQCSRTAYHRNFVDHMD | |
| | | DVYTTGPFGGGTGSMLFTLSFFYHEFKKHHCKWSASNR | |
| | | DFDLCRYRGTVLKFYRHPDVDYIVWLNRNPPFQENLLDA | |
| | | MSRQPLIMLQTHKCILVRSFKTHPRGPSYVRMKVRPPRL | |
| | | LTDKWYFQSDFCNVPLFQLQFALAELRFPIGSPQTNTTC | |
| | | VNFLVLDNRYHLFLDNKPRQSENLQRKERGHGYVFTGN | |
| | | EGEDDRLKFWHSLWSTGRFLNTTHINTLLPNISKLQDHEA | |
| | | EDTQAKTDYKSLINGNKKVYNDSQYMQDVWEQKKIQTLY | |
| | | KVIAEEQYRKIEKYYNTTYGQYQRQLFTGKKYWDYRVGM | |
| | | FSPTFLSPSRLNPEMPGAYTEIAYNPWTDEGTGNVVCLQ | |
| | | YLTKETSDYKPHAGSKFTIEDVPLWIAMNGYVDICKKEGK | |
| | | DPGIRLNCLMCIRCPYTRPKLYNPRYPEELFVVYSYNFAH | |
| | | GRMPGGDKYIPMEFKDRWYPSLMHQEEVIEDIVRSGPFA | |
| | | LKDQTEMVTCMMRYSALFNWGGNIIREQAVEDPCKKNT | |
| | | FALPGASGVARLLQVSNPIRQTPSTTWHSWDWRRSLFT | |
| | | QTGIKRMREQQPYDEITYAGPKRPKLTVPAGPTLAAGDA | |
| | | YNYWERKPLTSPGETLPTQTETETEAPEEEAQQEEVQE | |
| | | GLQLQQLWEQQLQQKRQLGVMFQQLLRLRTGAEIHPAL | |
| | | A* | |
| FJ392114.1 | ACR20272.1 | MAAWWWGRRRRWRRWRRRRLPRRRRWRRRRRWPR | 288 |
| | | RRRRRWPRRRRRRGPARRLRRRRRRRRVRRPRRRQKL | |
| | | VLTQWNPQTQRKCVVRGFLPLFFCGQGAYHRNFVEHM | |
| | | DDVFPKGPSGGGFGSMVWNLDFLYQEFKKHHNKWSSS | |
| | | NRDFDLVRCHGTVIKFYRHSDFDYLVHVTRTPPFKEDLLT | |
| | | IVSHQPGLMMQNYRCILVKSYKTHPGGRPYITPKIRPPRL | |
| | | LTDKWYFRPDFCGVPLFKLYVTLAELRFPICSPQTDTNCV | |
| | | TFLVLDNTYYDYLDNTADTTRDHERQQKWTNMKMTPRY | |
| | | HLTSHINTLFSGTQQMQSAKETGKDSQFRENIWKTAEVV | |
| | | KIIKDIASKNMQKQQTYYTKTYGAYATQYFTGKQYWDWR | |
| | | VGLFSPIFLSPSRLNPQEPGAYTEIAYNPWTDEGTGNIVCI | |
| | | QYLTKKDSHYKPGAGSKFAVTDVPLWAALFGYYDQCKK | |
| | | ESKDANIRLNRLLLVRCPYTRPKLYNPRDPDQLFVMYSY | |
| | | NFGHGRMPGGDKYVPMEFKDRWYPCMLHQEEVVEEIV | |
| | | RCGPFAPKDMTPSVTCMARYSSLFTWGGNIIREQAVEDP | |
| | | CKKSTFAIPGAGGLARILQVSNPQRQAPTTTWHSWGWR | |
| | | RSLFTETGLKRMQEQQPYDEMSYTGPKRPKLSVPPAAE | |
| | | GNLAAGGGLFFRDGKQPASPGGSLPTQSETEAEAEDEE | |
| | | AHQEETEEGAQLQQLWEQQLQQKRELGIVFQHLLRLRQ | |
| | | GAEIHPGLV* | |
| FJ392115.1 | ACR20274.1 | MAAWWWGRRRRWRRWRRRRXPRRRRWRRRRRWPR | 289 |
| | | RRRRRWPRRRRRRRPARRLRRRRRRRRVRRPRRRQKL | |
| | | VLTQWNPQTQRKCVVRGFLPLFFCGQGAYHRNFVEHM | |
| | | DDVFPKGPSGGGFGSMVWNLDFLYQEFKKHHNRWSSS | |
| | | NRDFDLVRYHGTVIKFYRHSDFDYLVHVTRTPPFKEDLLT | |
| | | IVSHQPGLMMQNYRCILVKSYKTHPGGRPYITLKIRPPRL | |
| | | LTDKWYFQPDFCGVPLFKLYVTLAELRFPICSPQTDTNCV | |
| | | TFLVLDNTYYDYLDSTADTTRDNERHQKWKNMIMTPRYH | |
| | | LTSHINTLFSGTQQMQNAKETGKDSQFRENIWKTEEVVKI | |
| | | IHDIASRNMQKQITYYTKTYGAYATQYFTGKQYWDWRVG | |
| | | LFSPIFLSPSRLNPQEPGAYTEIAYNPWTDEGTGNIVCIQY | |
| | | LTKKDSHYKPGAGSKFAVTDVPLWAALFGYYDQCKKES | |
| | | KDANIRLNCLLLVRCPYTRPKLYNPRDPDQLFVMYSYNF | |
| | | GHGRMPGGDKYVPMEFKDRWYPCMLHQEEVVEEIVRC | |
| | | GPFAPKDMTPSVTCMARYSSLFTWGGNIIREQAVEDPCK | |
| | | KSTFAIPGAGGLARILQVSNPQRQAPTTTWHLWDWRRSL | |
| | | FTETGLKRMQEQQPYDEMSYTGPKRPKLSVPPAAEGNL | |
| | | AAGGGLFFRDRKQPTSPGGSLPTQSETEAEAEDEEAHQ | |
| | | EETEEGAQLQQLWEQQLQQKRELGIVFQHLLRLRQGAEI | |
| | | HPGLV* | |
| FJ392117.1 | ACR20277.1 | MAWWWWRRRRRPWRRRWRWKRRARVRTRRPRRAVR | 290 |
| | | RRRRRVRRRRRGWRRLYRRWRRKGRRRRRRKKLVMK | |
| | | QWNPSTVSRCYIVGYLPIIIMGQGTASMNYASHSDDVYY | |
| | | PGPFGGGISSMRFTLRILYDQFMRGQNFWTKTNEDLDLA | |
| | | RFLGSKWRFYRHKDVDFIVTYETSAPFTDSLESGPHQHP | |
| | | GIQMLMKNKILIPSFATKPKGRSSIKVRIQPPKLMIDKWYP | |
| | | QTDFCEVTLLTIHATACNLRFPFCSPQTDTSCVQFQVLSY | |
| | | NAYRQRISILPELCTREKLREFIKQVVKPNLTCINTLATPW | |
| | | CFKFPELDKLPPVANNATGWSVNPDSGDGDVIYQETTLE | |
| | | TKWIANNDVWHTKDQRAHNNIHSQYGMPQSDALEHKTG | |
| | | YFSPALLSPQRLNPQIPGLYINIVYNPLTDKGEGNKIWCDP | |
| | | LTKNTFGYDPPKSKFLIENLPLWSAVTGYVDYCTKASKDE | |
| | | SFKYNYRVLIQTPYTVPALYSDSETTKNRGYIPIGTDFAYG | |
| | | RMPGGVQQIPIRWRMRWYPMLFNQQPVLEDLFQSGPFA | |
| | | YQGDAKSATLVGKYAFKWLWGGNRIFQQVVRDPRSHQ | |
| | | QDQSVGPSRQPRAVQVFDPKYQAPQWTFHAWDIRRGL | |
| | | FGRQAIKRVSAKPTPDELISTGPKRPRLEVPAFQEEQEKD | |
| | | LLFRQRKHKAWEDTTEEETEAPSEEEEENQELQLVRRLQ | |
| | | QQRELGRGLROLFQQLTRTQMGLHVDPQLLAPV* | |
| GU797360.1 | ADO51761.1 | MAWGWWKRRRKWWWRRRWTRGRLRKRRARRAGRR | 291 |
| | | PRRRRVRRRRAWRRGRRKRRTFRRRRRRKGRRHRTRL | |
| | | IIRQWQPEIVRKCLIIGYFPMIICGQGRWSENYSSHLEDRV | |
| | | VKQAFGGGHATTRWSLKVLYEENLRHLNFWIWTNRDLE | |
| | | LARYLKVTWTFYRHQDVDFIIYFNRKSPMGGNIYTAPMM | |
| | | HPGALMLSKHKILVKSFKTKPKGKATVKVTIKPPTLLVDK | |
| | | WYFQKDICDMTLLNLNAVAADLRFPFCSPQTDNPCINFQ | |
| | | VLSSVYNNFLSITDNRLTPVTDDGQAYYKAFLDAAFTKDR | |
| | | DFNAVNTFRTISNFSHPQLELPTKTTNTSQDQYFNTLDGY | |
| | | WGDPIYVHTQNIKPDQNLDKCKEILTNNMKNWHKKVKSE | |
| | | NPSSLNHSCFAHNVGIFSSSFLSAGRLAPEVPGLYTDVIY | |
| | | NPYTDKGKGNMLWVDYCSKGDNLYKEGQSKCLLANLPL | |
| | | WMATNGYIDWVKKETDNWVINTQARVLMVCPYTYPKLY | |
| | | HEIQPLYGFVVYSYNFGEGKMPNGATYIPFKFRNKWYPTI | |
| | | YMQQAVLEDISRSGPFALKQQIPSATLTAKYKFKFLFGGN | |
| | | PTSEQVVRDPCTQPTFELPGASTQPPRIQVTDPKLLGPH | |
| | | YSFHSWDLRRGYYSTKSIKRMSEHEEPSEFIFPGPKKPR | |
| | | VDLGPIQQQERPSDSLQRESRPWETSEEESEAEVQQEE | |
| | | TEEVPLRQQLLHNLREQQQLRKGLQCVFQQLIKTQQGVH | |
| | | IDPSLL* | |
| DQ003341.1 | AAX94182.1 | MAWSWWWRRRKRWWPRRRRRWRRFRTRRARRAVPR | 292 |
| | | RRRRRRVRRRRWGRRGRRRRVFYKRRRRKTGRLYRKP | |
| | | KKKLVLTQWHPTTVRNCSIRGLVPLVLCGHTQGGRNFAL | |
| | | RSDDYPKQGSPYGGSFSTTTWNLRVLFDEHQKHHNTW | |
| | | SYPNNQLDLGRYKGCTFCFYRGKKTDYIVKFQRRGPFKI | |
| | | NKYSSPMAHPGMMMLDKMKILVPSFDTRPGGR* | |
| DQ003342.1 | AAX94185.1 | MAWSWWWRRRKRWWPRRRRRWRRFRTRRARRAVPR | 293 |
| | | RRRRRRVRRRRWGRRGRRRRVFYKRRRRKTGRLYRKP | |
| | | KKKLVLTQWHPTTVRNCSIRGLVPLVLCGHTQGGRNFAL | |
| | | RSDDYPKQGSPYGGSFSTTTWNLRVLFDEHQKHHNTW | |
| | | SYPNNQLDLGRYKGCTFCFYRGKKTDYIVKFQRRGPFKI | |
| | | NKYSSPMAHPGMMMLDKMKILVPSFDTRPGGR* | |
| DQ003343.1 | AAX94188.1 | MAWSWWWRRRKRWWPRRRRRWRRFRTRRARRAVPR | 294 |
| | | RRRRRRVRRRRWGRRRRRRRVFYKRRRRKTGRLYRKP | |
| | | KKKLVLTQWHPTTVRNCSIRGLVPLVLCGHTQGGRNFAL | |
| | | RSDDYPKQGSPYGGSFSTTTWNLRVLFDEHQKHHNTW | |
| | | SYPNNQLDLGRYKGCTFYFYRDKKTDYIVKFQRRGPFKI | |
| | | NKYSSPMAHPGMMMLDKMKILVPSFDTRPGGR* | |
| DQ003344.1 | AAX94191.1 | MAWSWWWRRRKRWWPRRRRRWRRFRTRRARRAVPR | 295 |
| | | RRRRRRVRRRRWGRRRRRRRVFYKRRRRKTGRLYRKP | |
| | | KKKLVLTQWHPTTVRNCSIRGLVPLVLCGHTQGGRNFAL | |
| | | RSDDYPKQGSPYGGSFSTTTWNLRVLFDEHQKHHNTW | |
| | | SYPNNQLDLGRYKGCTFYFYRDKKTDYIVKFQRRGPFKI | |
| | | NKYSSPMAHPGMMMLDKMKILVPSFDTRPGGR* | |
| DQ003341.1 | AAX94183.1 | MYYGCIGINSTLTTKYENLFNKLYSKCCYFETFQTIAQLNP | 296 |
| | | GFKAAKKTTNGSGSTAATLGDAVTELKNPNGTFYTGNNS | |
| | | TFGCCTYKPTKQIGSNANKWFWHQLTATDSDTLGQYGR | |
| | | ASIQYMEYHTGIYSSIFLSPLRSNLELPTAYQDVTYNPLTD | |
| | | RGIGNRIWYQYSTKENTTFNETQCKCVLSDLPLWSMFYG | |
| | | YVDFIESELGISAEIHNFGIVCVQCPYTFPPMFDKSKPDKG | |
| | | YVFYDTLFGNGKMPDGSGHVPTYWQQRWWPRFSFQR | |
| | | QVMHDIILTGPFSYKDDSVMTGITAGYKFKFSWGGDMVS | |
| | | EQVIKNPERGDGRDSTYPDRQRRDSQVVDPRSMGPQW | |
| | | VFHTFDYRRGLFGKDAIKRVSEKPTDPDYFTTPYKKPRFF | |
| | | PPTAGEEKLQEEDSALQEKRSPLSSEEGQTRAQVLQQQ | |
| | | VLQSELQQQQELGEQLRFLLREMFKTQAGIHMNPRAFQ | |
| | | EL* | |
| DQ003342.1 | AAX94186.1 | MYYGCIGINSTLTTKYENLFNKLYSKCCYFETFQTIAQLNP | 297 |
| | | GFKAAKKTTNGSGSTAATLGDAVTELKNPNGTFYTGNNS | |
| | | TFGCCTYKPTKQIGSNANKWFWHQLTATDSDTLGQYGR | |
| | | ASIQYMEYHTGIYSSIFLSPLRSNLELPTAYQDVTYNPLTD | |
| | | RGIGNRIWYQYSTKENTTFNETQCKCVLSDLPLWSMFYG | |
| | | YVDFIESELGISAEIHNFGIVCVQCPYTFPPMFDKSKPDKG | |
| | | YVFYDTLFGNGKMPDGSGHVPTYWQQRWWPRFSFQR | |
| | | QVMHDIILTGPFSYKDDSVMTGITAGYKFKFSWGGDMVS | |
| | | EQVIKNPERGDGRDSTYPDRQRRDSQVVDPRSMGPQW | |
| | | VFHTFDYRRGLFGKDAIKRVSEKPTDPDYFTTPYKKPRFF | |
| | | PPTAGEEKLQEEDSALQEKRSPLSSEEGQTRAQVLQQQ | |
| | | VLQSELQQQQELGEQLRFLLREMFKTQAGIHMNPRAFQ | |
| | | EL* | |
| DQ003343.1 | AAX94189.1 | MYYDCIGINSTLTTKYENLFNKLYSKCCYFETFQTIAQLNP | 298 |
| | | GFKAAKKTTNGSGSTAATLGDAVTELKNPNGTFYTGNNS | |
| | | TFGCCTYKPTKQIGSNANKWFWHQLTATDSDTLGQYGR | |
| | | ASIQYMEYHTGIYSSIFLSPLRSNLEFPTAYQDVTYNPLTD | |
| | | RGIGNRIWYQYSTKENTTFNETQCKCVLSDLPLWSMFYG | |
| | | YVDFIESELGISAEIHNFGIVCVQCPYTFPPMFDKSKPDKG | |
| | | YVFYDTLFGNGKMPDGSGHVPTYWQQRWWPRFSFQR | |
| | | QVMHDIILTGPFSYKDDSVMTGITAGYKFKFSWGGDMVS | |
| | | EQVIKNSERGDGRDSTYPDRQRRDLQVVDPRSMGPQW | |
| | | VFHTFDYRRGLFGKDAIKRVSEKPTDPDYFTTPYKKPRFF | |
| | | PPTAGEEKLQEEDSALQEKRSPLSSEEGQTRAQVLQQQ | |
| | | VLQSELQQQQELGEQLRFLLREMFKTQAGIHMNPRAFQ | |
| | | EL* | |
| DQ003344.1 | AAX94192.1 | MYYDCIGINSTLTTKYENLFNKLYSKCCYFETFQTIAQLNP | 299 |
| | | GFKAAKKTTNGSGSTAATLGDAVTELKNPNGTFYTGNNS | |
| | | TFGCCTYKPTKQIGSNANKWFWHQLTATDSDTLGQYGR | |
| | | ASIQYMEYHTGIYSSIFLSPLRSNLEFPTAYQDVTYNPLTD | |
| | | RGIGNRIWYQYSTKENTTFNETQCKCVLSDLPLWSMFYG | |
| | | YVDFIESELGISAEIHNFGIVCVQCPYTFPPMFDKSKPDKG | |
| | | YVFYDTLFGNGKMPDGSGHVPTYWQQRWWPRFSFQR | |
| | | QVMHDIILTGPFSYKDDSVMTGITAGYKFKFSWGGDMVS | |
| | | EQVIKNSERGDGRDSTYPDRQRRDLQVVDPRSMGPQW | |
| | | VFHTFDYRRGLFGKDAIKRVSEKPTDPDYFTTPYKKPRFF | |
| | | PPTAGEEKLQEEDSALQEKRSPLSSEEGQTRAQVLQQQ | |
| | | VLQSELQQQQELGEQLRFLLREMFKTQAGIHMNPRAFQ | |
| | | EL* | |

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., Table 17. In some embodiments, the substantially non-pathogenic protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences described herein, e.g., as listed in any of Tables 2, 4, 6, 8, 10, 12, 14, 16, or 17.

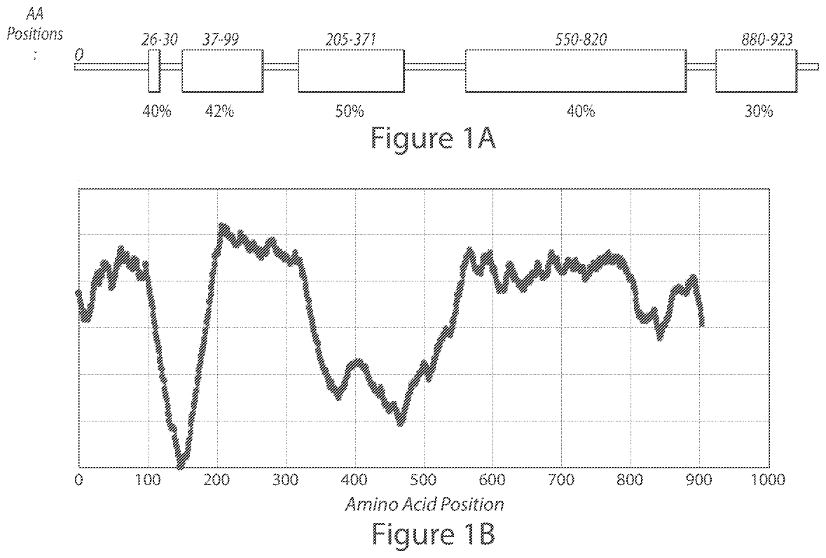

In some embodiments, the genetic element comprises a nucleotide sequence encoding an amino acid sequence having about position 1 to about position 150 (e.g., or any subset of amino acids within each range, e.g., about position 20 to about position 35, about position 25 to about position 30, about position 26 to about 30), about position 150 to about position 390 (e.g., or any subset of amino acids within each range, e.g., about position 200 to about position 380, about position 205 to about position 375, about position 205 to about 371), about 390 to about position 525, about position 525 to about position 850 (e.g., or any subset of amino acids within each range, e.g., about position 530 to about position 840, about position 545 to about position 830, about position 550 to about 820), about 850 to about position 950 (e.g., or any subset of amino acids within each range, e.g., about position 860 to about position 940, about position 870 to about position 930, about position 880 to about 923) of the amino acid sequences described herein, e.g., as listed in any of Tables 2, 4, 6, 8, 10, 12, 14, 16, or 17, or shown in FIG. 1, or a functional fragment thereof. In some embodiments, the substantially non-pathogenic protein comprises an amino acid sequence or a functional fragment thereof or a sequence having at least about 60%, 65%, 70%, 75%, 80%, 85%, 90% 95%, 96%, 97%, 98%, 99%, or 100% sequence identity to about position 1 to about position 150 (e.g., or any subset of amino acids within each range as described herein), about position 150 to about position 390, about position 390 to about position 525, about position 525 to about position 850, about position 850 to about position 950 of the amino acid sequences described herein, e.g., as listed in any of Tables 2, 4, 6, 8, 10, 12, 14, 16, or 17, or as shown in FIG. 1.

In some embodiments, the substantially non-pathogenic protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences or ranges of amino acids described herein, e.g., as listed in any of Tables 2, 4, 6, 8, 10, 12, 14, 16, or 17, or shown in FIG. 1, where the sequence is a functional domain or provides a function, e.g., species and/or tissue and/or cell tropism, viral genome binding and/or packaging, immune evasion (non-immunogenicity and/or tolerance), pharmacokinetics, endocytosis and/or cell attachment, nuclear entry, intracellular modulation and localization, exocytosis modulation, propagation, nucleic acid protection, and a combination thereof. In some embodiments, the ranges of amino acids with less sequence identity may provide one or more of the properties described herein and differences in cell/tissue/species specificity (e.g. tropism).

Protein Binding Sequence

A strategy employed by many viruses is that the viral capsid protein recognizes a specific protein binding sequence in its genome. For example, in viruses with unsegmented genomes, such as the L-A virus of yeast, there is a secondary structure (stem-loop) and a specific sequence at the 5′ end of the genome that are both used to bind the viral capsid protein. However, viruses with segmented genomes, such as Reoviridae, Orthomyxoviridae (influenza), Bunyaviruses and Arenaviruses, need to package each of the genomic segments. Some viruses utilize a complementarity region of the segments to aid the virus in including one of each of the genomic molecules. Other viruses have specific binding sites for each of the different segments. See for example, Curr Opin Struct Biol. 2010 February; 20(1): 114-120; and Journal of Virology (2003), 77(24), 13036-13041.

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds an arginine-rich region of the substantially non-pathogenic protein. In some embodiments, the genetic element comprises a protein binding sequence as described in Example 8. In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus sequence (e.g., as shown in any of Tables 1, 3, 5, 7, 9, 11, or 13). In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of Table 1 (e.g., nucleotides 177-247 of the nucleic acid sequence of Table 1). In embodiments, the protein binding sequence has at least about 70%, 75%, 80%, 85%, 90%, 95%, 96%, 97%, 98%, 99%, or 100% sequence identity to the Anellovirus GC-rich nucleotide sequence of Table 1 (e.g., nucleotides 3415-3570 of the nucleic acid sequence of Table 1). In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of Table 3 (e.g., nucleotides 174-244 of the nucleic acid sequence of Table 3). In embodiments, the protein binding sequence has at least about 70%, 75%, 80%, 85%, 90%, 95%, 96%, 97%, 98%, 99%, or 100% sequence identity to the Anellovirus GC-rich nucleotide sequence of Table 3 (e.g., nucleotides 3691-3794 of the nucleic acid sequence of Table 3). In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of Table 5 (e.g., nucleotides 170-240 of the nucleic acid sequence of Table 5). In embodiments, the protein binding sequence has at least about 70%, 75%, 80%, 85%, 90%, 95%, 96%, 97%, 98%, 99%, or 100% sequence identity to the Anellovirus GC-rich nucleotide sequence of Table 5 (e.g., nucleotides 3632-3753 of the nucleic acid sequence of Table 5). In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of Table 7 (e.g., nucleotides 174-244 of the nucleic acid sequence of Table 7). In embodiments, the protein binding sequence has at least about 70%, 75%, 80%, 85%, 90%, 95%, 96%, 97%, 98%, 99%, or 100% sequence identity to the Anellovirus GC-rich nucleotide sequence of Table 7 (e.g., nucleotides 3733-3853 of the nucleic acid sequence of Table 7). In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of Table 9 (e.g., nucleotides 171-241 of the nucleic acid sequence of Table 9). In embodiments, the protein binding sequence has at least about 70%, 75%, 80%, 85%, 90%, 95%, 96%, 97%, 98%, 99%, or 100% sequence identity to the Anellovirus GC-rich nucleotide sequence of Table 9 (e.g., nucleotides 3644-3758 of the nucleic acid sequence of Table 9). In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of Table 11 (e.g., nucleotides 323-393 of the nucleic acid sequence of Table 11). In embodiments, the protein binding sequence has at least about 70%, 75%, 80%, 85%, 90%, 95%, 96%, 97%, 98%, 99%, or 100% sequence identity to the Anellovirus GC-rich nucleotide sequence of Table 11 (e.g., nucleotides 2868-2929 of the nucleic acid sequence of Table 11). In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of Table 13 (e.g., nucleotides 117-187 of the nucleic acid sequence of Table 13). In embodiments, the protein binding sequence has at least about 70%, 75%, 80%, 85%, 90%, 95%, 96%, 97%, 98%, 99%, or 100% sequence identity to the Anellovirus GC-rich nucleotide sequence of Table 13 (e.g., nucleotides 3054-3172 of the nucleic acid sequence of Table 13).

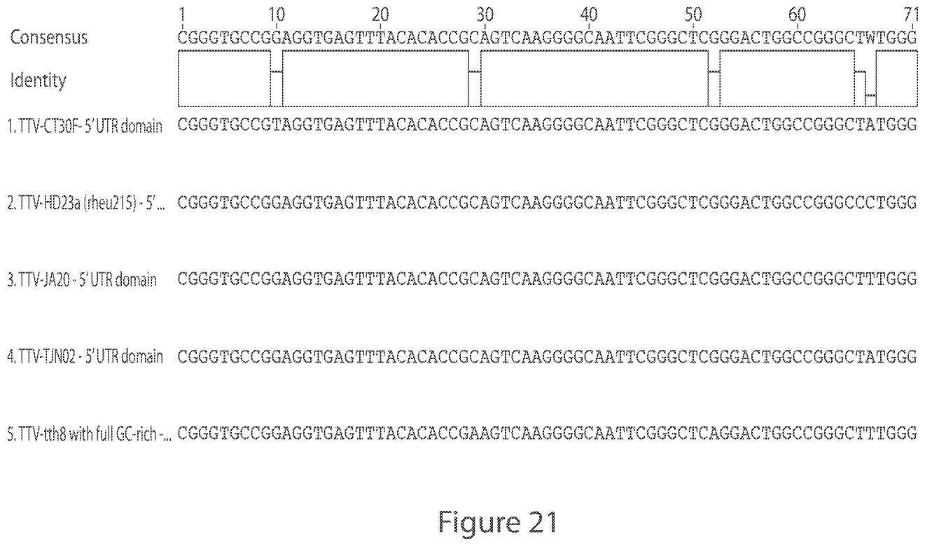

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 16-1 and/or FIG. 21. In some embodiments, the genetic element (e.g., protein-binding sequence of the genetic element) comprises a nucleic acid sequence of the Consensus 5′ UTR sequence shown in Table 16-1, wherein X₁, X₂, X₃, X₄, and X₅ are each independently any nucleotide, e.g., wherein X₁=G or T, X₂=C or A, X₃=G or A, X₄=T or C, and X₅=A, C, or T). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus 5′ UTR sequence shown in Table 16-1. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the exemplary TTV 5′ UTR sequence shown in Table 16-1. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-CT30F 5′ UTR sequence shown in Table 16-1. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-HD23a 5′ UTR sequence shown in Table 16-1. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-JA20 5′ UTR sequence shown in Table 16-1. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-TJN02 5′ UTR sequence shown in Table 16-1. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-tth8 5′ UTR sequence shown in Table 16-1.

TABLE 16-1
Exemplary 5′ UTR sequences from Anelloviruses

| Source | Sequence | SEQ ID NO: |
|---|---|---|
| Consensus | CGGGTGCCGX₁AGGTGAGTTTACACACCGX₂AGT | 715 |
| | CAAGGGGCAATTCGGGCTCX₃GGACTGGCCGGG | |
| | CX₄X₅TGGG | |
| | X₁ = G or T | |
| | X₂ = C or A | |
| | X₃ = G or A | |
| | X₄ = T or C | |
| | X₅ = A, C, or T | |
| Exemplary TTV | CGGGTGCCGGAGGTGAGTTTACACACCGCAGTC | 703 |
| Sequence | AAGGGGCAATTCGGGCTCGGGACTGGCCGGGCT | |
| | WTGGG | |
| TTV-CT30F | CGGGTGCCGTAGGTGAGTTTACACACCGCAGTC | 704 |
| | AAGGGGCAATTCGGGCTCGGGACTGGCCGGGCT | |
| | ATGGG | |
| TTV-HD23a | CGGGTGCCGGAGGTGAGTTTACACACCGCAGTC | 705 |
| | AAGGGGCAATTCGGGCTCGGGACTGGCCGGGCC | |
| | CTGGG | |
| TTV-JA20 | CGGGTGCCGGAGGTGAGTTTACACACCGCAGTC | 706 |
| | AAGGGGCAATTCGGGCTCGGGACTGGCCGGGCT | |
| | TTGGG | |
| TTV-TJN02 | CGGGTGCCGGAGGTGAGTTTACACACCGCAGTC | 707 |
| | AAGGGGCAATTCGGGCTCGGGACTGGCCGGGCT | |
| | ATGGG | |
| TTV-tth8 | CGGGTGCCGGAGGTGAGTTTACACACCGAAGTC | 708 |
| | AAGGGGCAATTCGGGCTCAGGACTGGCCGGGCT | |
| | TTGGG | |

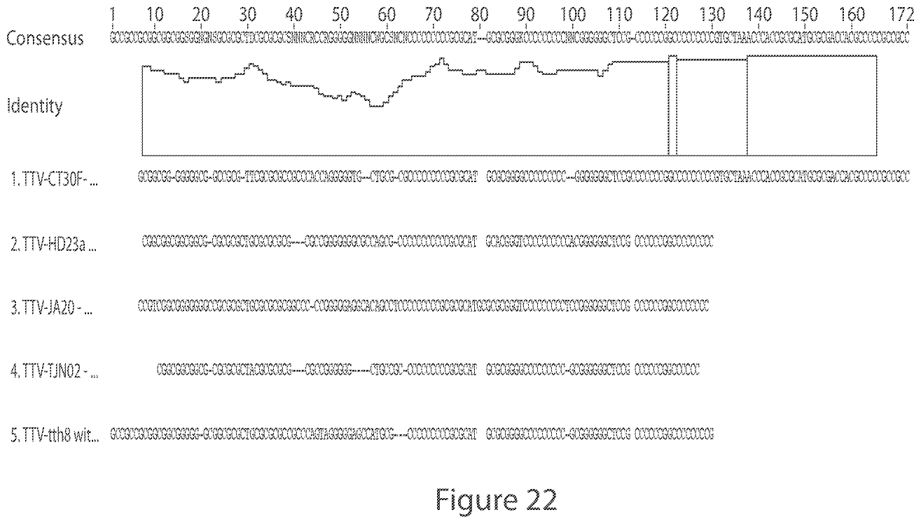

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 16-2 and/or FIG. 22. In embodiments, the genetic element (e.g., protein-binding sequence of the genetic element) comprises a nucleic acid sequence of the Consensus GC-rich sequence shown in Table 16-1, wherein X₁, X₄, X₅, X₆, X₇, X₁₂, X₁₃, X₁₄, X₁₅, X₂₀, X₂₁, X₂₂, X₂₆, X₂₉, X₃₀, and X₃₃ are each independently any nucleotide and wherein X₂, X₃, X₅, X₉, X₁₀, X₁₁, X₁₆, X₁₇, X₁₈, X₁₉, X₂₃, X₂₄, X₂₅, X₂₇, X₂₈, X₃₁, X₃₂, and X₃₄ are each independently absent or any nucleotide. In some embodiments, one or more of (e.g., all of) X₁ through X₃₄ are each independently the nucleotide (or absent) specified in Table 16-2. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus GC-rich sequence shown in Table 16-1. In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an exemplary TTV GC-rich sequence shown in Table 16-1 (e.g., the full sequence, Fragment 1, Fragment 2, Fragment 3, or any combination thereof, e.g., Fragments 1-3 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-CT30F GC-rich sequence shown in Table 16-1 (e.g., the full sequence, Fragment 1, Fragment 2, Fragment 3, Fragment 4, Fragment 5, Fragment 6, Fragment 7, Fragment 8, or any combination thereof, e.g., Fragments 1-7 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-HD23a GC-rich sequence shown in Table 16-1 (e.g., the full sequence, Fragment 1, Fragment 2, Fragment 3, Fragment 4, Fragment 5, Fragment 6, or any combination thereof, e.g., Fragments 1-6 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-JA20 GC-rich sequence shown in Table 16-1 (e.g., the full sequence, Fragment 1, Fragment 2, or any combination thereof, e.g., Fragments 1 and 2 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-TJN02 GC-rich sequence shown in Table 16-1 (e.g., the full sequence, Fragment 1, Fragment 2, Fragment 3, Fragment 4, Fragment 5, Fragment 6, Fragment 7, Fragment 8, or any combination thereof, e.g., Fragments 1-8 in order). In embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-tth8 GC-rich sequence shown in Table 16-1 (e.g., the full sequence, Fragment 1, Fragment 2, Fragment 3, Fragment 4, Fragment 5, Fragment 6, or any combination thereof, e.g., Fragments 1-6 in order).

TABLE 16-2
Exemplary GC-rich sequences from Anelloviruses

| | | | SEQ ID |
|---|---|---|---|
| Source | | Sequence | NO: |
| Consensus | | CGGCGGX₁GGX₂GX₃X₄X₅CGCGCTX₆CG | 743 |
| | | CGCGCX₇X₈X₉X₁₀CX₁₁X₁₂X₁₃X₁₄GGGGX₁₅ | |
| | | X₁₆X₁₇X₁₈X₁₉X₂₀X₂₁GCX₂₂X₂₃X₂₄X₂₅CCCCC | |
| | | CCX₂₆CGCGCATX₂₇X₂₈GCX₂₉CGGGX₃₀CC | |
| | | CCCCCCCX₃₁X₃₂X₃₃GGGGGGCTCCGX₃₄C | |
| | | CCCCCGGCCCCCC | |
| | | X₁ = G or C | |
| | | X₂ = G, C, or absent | |
| | | X₃ = C or absent | |
| | | X₄ = G or C | |
| | | X₅ = G or C | |
| | | X₆ = T, G, or A | |
| | | X₇ = G or C | |
| | | X₈ = G or absent | |
| | | X₉ = C or absent | |
| | | X₁₀ = C or absent | |
| | | X₁₁ = G, A, or absent | |
| | | X₁₂ = G or C | |
| | | X₁₃ = C or T | |
| | | X₁₄ = G or A | |
| | | X₁₅ = G or A | |
| | | X₁₆ = A, G, T, or absent | |
| | | X₁₇ = G, C, or absent | |
| | | X₁₈ = G, C, or absent | |
| | | X₁₉ = C, A, or absent | |
| | | X₂₀ = C or A | |
| | | X₂₁ = T or A | |
| | | X₂₂ = G or C | |
| | | X₂₃ = G, T, or absent | |
| | | X₂₄ = C or absent | |
| | | X₂₅ = G, C, or absent | |
| | | X₂₆ = G or C | |
| | | X₂₇ = G or absent | |
| | | X₂₈ = C or absent | |
| | | X₂₉ = G Or A | |
| | | X₃₀ = G or T | |
| | | X₃₁ = C, T, or absent | |
| | | X₃₂ = G, C, A, or absent | |
| | | X₃₃ = G Or C | |
| | | X₃₄ = C or absent | |
| Exemplary TTV | Full sequence | GCCGCCGCGGCGGCGGSGGNGNSGCG | 709 |
| Sequence | | CGCTDCGCGCGCSNNNCRCCRGGGGGN | |
| | | NNNCWGCSNCNCCCCCCCCCGCGCAT | |
| | | GCGCGGGKCCCCCCCCCNNCGGGGGG | |
| | | CTCCGCCCCCCGGCCCCCCCCCGTGCT | |
| | | AAACCCACCGCGCATGCGCGACCACG | |
| | | CCCCCGCCGCC | |
| | Fragment 1 | GCCGCCGCGGCGGCGGSGGNGNSGCG | 716 |
| | | CGCTDCGCGCGCSNNNCRCCRGGGGGN | |
| | | NNNCWGCSNCNCCCCCCCCCGCGCAT | |
| | Fragment 2 | GCGCGGGKCCCCCCCCCNNCGGGGGG | 717 |
| | | CTCCG | |
| | Fragment 3 | CCCCCCGGCCCCCCCCCGTGCTAAACC | 718 |
| | | CACCGCGCATGCGCGACCACGCCCCCG | |
| | | CCGCC | |
| TTV-CT30F | Full sequence | GCGGCGG-GGGGGCG-GCCGCG- | 710 |
| | | TTCGCGCGCCGCCCACCAGGGGGTG-- | |
| | | CTGCG-CGCCCCCCCCCGCGCAT | |
| | | GCGCGGGGCCCCCCCCC-- | |
| | | GGGGGGGCTCCGCCCCCCCGGCCCCCC | |
| | | CCCGTGCTAAACCCACCGCGCATGCGC | |
| | | GACCACGCCCCCGCCGCC | |
| | Fragment 1 | GCGGCGG | 719 |
| | Fragment 2 | GGGGGCG | 720 |
| | Fragment 3 | GCCGCG | 721 |
| | Fragment 4 | TTCGCGCGCCGCCCACCAGGGGGTG | 722 |
| | Fragment 5 | CTGCG | 723 |
| | Fragment 6 | CGCCCCCCCCCGCGCAT | 724 |
| | Fragment 7 | GCGCGGGGCCCCCCCCC | 725 |
| | Fragment 8 | GGGGGGGCTCCGCCCCCCCGGCCCCCC | 726 |
| | | CCCGTGCTAAACCCACCGCGCATGCGC | |
| | | GACCACGCCCCCGCCGCC | |
| TTV-HD23a | Full sequence | CGGCGGCGGCGGCG- | 711 |
| | | CGCGCGCTGCGCGCGCG--- | |
| | | CGCCGGGGGGGCGCCAGCG- | |
| | | CCCCCCCCCCCGCGCAT | |
| | | GCACGGGTCCCCCCCCCCACGGGGGGC | |
| | | TCCG CCCCCCGGCCCCCCCCC | |
| | Fragment 1 | CGGCGGCGGCGGCG | 727 |
| | Fragment 2 | CGCGCGCTGCGCGCGCG | 728 |
| | Fragment 3 | CGCCGGGGGGGCGCCAGCG | 729 |
| | Fragment 4 | CCCCCCCCCCCGCGCAT | 730 |
| | Fragment 5 | GCACGGGTCCCCCCCCCCACGGGGGGC | 731 |
| | | TCCG | |
| | Fragment 6 | CCCCCCGGCCCCCCCCC | 732 |
| TTV-JA20 | Full sequence | CCGTCGGCGGGGGGGCCGCGCGCTGC | 712 |
| | | GCGCGCGGCCC- | |
| | | CCGGGGGAGGCACAGCCTCCCCCCCCC | |
| | | GCGCGCATGCGCGCGGGTCCCCCCCCC | |
| | | TCCGGGGGGCTCCGCCCCCCGGCCCCC | |
| | | CCC | |
| | Fragment 1 | CCGTCGGCGGGGGGGCCGCGCGCTGC | 733 |
| | | GCGCGCGGCCC | |
| | Fragment 2 | CCGGGGGAGGCACAGCCTCCCCCCCCC | 734 |
| | | GCGCGCATGCGCGCGGGTCCCCCCCCC | |
| | | TCCGGGGGGCTCCGCCCCCCGGCCCCC | |
| | | CCC | |
| TTV-TJN02 | Full sequence | CGGCGGCGGCG- | 713 |
| | | CGCGCGCTACGCGCGCG--- | |
| | | CGCCGGGGGG----CTGCCGC- | |
| | | CCCCCCCCCGCGCAT | |
| | | GCGCGGGGCCCCCCCCC-GCGGGGGGCTCCG | |
| | | CCCCCCGGCCCCCC | |
| | Fragment 1 | CGGCGGCGGCG | 735 |
| | Fragment 2 | CGCGCGCTACGCGCGCG | 736 |
| | Fragment 3 | CGCCGGGGGG | 737 |
| | Fragment 4 | CTGCCGC | 738 |
| | Fragment 5 | CCCCCCCCCGCGCAT | 739 |
| | Fragment 6 | GCGCGGGGCCCCCCCCC | 740 |
| | Fragment 7 | GCGGGGGGCTCCG | 741 |
| | Fragment 8 | CCCCCCGGCCCCCC | 742 |
| TTV-tth8 | Full sequence | GCCGCCGCGGCGGCGGGGG- | 714 |
| | | GCGGCGCGCTGCGCGCGCCGCCCAGTA | |
| | | GGGGGAGCCATGCG--- | |
| | | CCCCCCCCCGCGCAT | |
| | | GCGCGGGGCCCCCCCCC- | |
| | | GCGGGGGGCTCCG | |
| | | CCCCCCGGCCCCCCCCG | |
| | Fragment 1 | GCCGCCGCGGCGGCGGGGG | 744 |
| | Fragment 2 | GCGGCGCGCTGCGCGCGCCGCCCAGTA | 745 |
| | | GGGGGAGCCATGCG | |
| | Fragment 3 | CCCCCCCCCGCGCAT | 746 |
| | Fragment 4 | GCGCGGGGCCCCCCCCC | 747 |
| | Fragment 5 | GCGGGGGGCTCCG | 748 |
| | Fragment 6 | CCCCCCGGCCCCCCCCG | 749 |

Effector

In some embodiments, the genetic element may include one or more sequences that encode a functional nucleic acid, e.g., an exogenous effector, e.g., a therapeutic, e.g., a regulatory nucleic acid, e.g., cytotoxic or cytolytic RNA or protein. In some embodiments, the functional nucleic acid is a non-coding RNA.

In some embodiments, the sequence encoding an exogenous effector is inserted into the genetic element, e.g., at an insert site as described in Example 10, 12, or 22. In embodiments, the sequence encoding an exogenous effector is inserted into the genetic element at a noncoding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In embodiments, the sequence encoding an exogenous effector is inserted into the genetic element at about nucleotide 3588 of a TTV-tth8 plasmid, e.g., as described herein or at about nucleotide 2843 of a TTMV-LY2 plasmid, e.g., as described herein. In embodiments, the sequence encoding an exogenous effector is inserted into the genetic element at or within nucleotides 336-3015 of a TTV-tth8 plasmid, e.g., as described herein, or at or within nucleotides 242-2812 of a TTV-LY2 plasmid, e.g., as described herein. In some embodiments, the sequence encoding an exogenous effector replaces part or all of an open reading frame (e.g., an ORF as described herein, e.g., an ORF1, ORF1/1, ORF1/2, ORF2, ORF2/2, ORF2/3, and/or ORF2t/3 as shown in any of Tables 1-14).

In some embodiments, the sequence encoding an exogenous effector comprises 100-2000, 100-1000, 100-500, 100-200, 200-2000, 200-1000, 200-500, 500-1000, 500-2000, or 1000-2000 nucleotides. In some embodiments, the exogenous effector is a nucleic acid or protein payload, e.g., as described in Example 11.

Regulatory Nucleic Acid

In some embodiments, the regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In embodiments, the regulatory nucleic acid encodes an miRNA.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acids, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

In some embodiments, the regulatory nucleic acid is at least one miRNA, e.g., 2, 3, 4, 5, 6, or more. In some embodiments, the genetic element comprises a sequence that encodes an miRNA at least about 75%, 80%, 85%, 90% 95%, 96%, 97%, 98%, 99% or 100% nucleotide sequence identity to any one of the nucleotide sequences or a sequence that is complementary to a sequence described herein, e.g., in Table 18.

TABLE 18
Examples of regulatory nucleic acids, e.g., miRNAs.

| Accession | Exemplary | | | | | | |
|---|---|---|---|---|---|---|---|
| number of | subsequence | | SEQ ID | miRNA_5prime_ | SEQ ID | miRNA_3prime_ | SEQ ID |
| strain | nucleotides | Pre_miRNA | NO: | per_MiRdup | NO: | per_MiRdup | NO: |
| AB008394.1 | AB008394_ | GCCAUUUUAAGUA | 300 | AGUAGCUGAC | 395 | CAUCCUCGGC | 490 |
| | 3475_3551 | GCUGACGUCAAGG | | GUCAAGGAUU | | GGAAGCUACA | |
| | | AUUGACGUAAAGG | | GAC(5′) | | CAA(3′) | |
| | | UUAAAGGUCAUCC | | | | | |
| | | UCGGCGGAAGCUA | | | | | |
| | | CACAAAAUGGU | | | | | |
| AB008394.1 | AB008394_ | GCGUACGUCACAA | 301 | CAAGUCACGU | 396 | GGCCCCGUCA | 491 |
| | 3579_3657 | GUCACGUGGAGGG | | GGAGGGGACC | | CGUGACUUAC | |
| | | GACCCGCUGUAAC | | CG(5′) | | CAC(3′) | |
| | | CCGGAAGUAGGCC | | | | | |
| | | CCGUCACGUGACU | | | | | |
| | | UACCACGUGUGUA | | | | | |
| AB017613.1 | AB017613_ | GCCAUUUUAAGUA | 302 | AAGUAGCUGA | 397 | UCAUCCUCGG | 492 |
| | 3462_3539 | GCUGACGUCAAGG | | CGUCAAGGAU | | CGGAAGCUAC | |
| | | AUUGACGUGAAGG | | UGACG(5′) | | ACAA(3′) | |
| | | UUAAAGGUCAUCC | | | | | |
| | | UCGGCGGAAGCUA | | | | | |
| | | CACAAAAUGGUG | | | | | |
| AB017613.1 | AB017613_ | GCACACGUCAUAA | 303 | AUAAGUCACG | 398 | GGCCCCGUCA | 493 |
| | 3566_3644 | GUCACGUGGUGGG | | UGGUGGGGAC | | CGUGAUUUGU | |
| | | GACCCGCUGUAAC | | CCG(5′) | | CAC(3′) | |
| | | CCGGAAGUAGGCC | | | | | |
| | | CCGUCACGUGAUU | | | | | |
| | | UGUCACGUGUGUA | | | | | |
| AB025946.1 | AB025946_ | CUUCCGGGUCAUA | 304 | UGGGGAGGGU | 399 | CCGGGUCAUA | 494 |
| | 3534_3600 | GGUCACACCUACG | | UGGCGUAUAG | | GGUCACACCU | |
| | | UCACAAGUCACGU | | CCCGGA(3′) | | ACGUCAC(5′) | |
| | | GGGGAGGGUUGGC | | | | | |
| | | GUAUAGCCCGGAA | | | | | |
| | | G | | | | | |
| AB025946.1 | AB025946_ | GCCGGGGGGCUGC | 305 | CCCCCCCCGG | 400 | GGCUGCCGCC | 495 |
| | 3730_3798 | CGCCCCCCCCGGG | | GGGGGGGUUU | | CCCCCCGGGG | |
| | | GAAAGGGGGGGGC | | GCCC(3′) | | AAAGGGGG(5′) | |
| | | CCCCCCCGGGGGG | | | | | |
| | | GGGUUUGCCCCCC | | | | | |
| | | CCC | | | | | |
| AB028668.1 | AB028668_ | AUACGUCAUCAGU | 306 | AUCAGUCACG | 401 | AUCCUCGUCC | 496 |
| | 3537_3615 | CACGUGGGGGAAG | | UGGGGGAAGG | | ACGUGACUGU | |
| | | GCGUGCCUAAACC | | CGUGC(5′) | | GA(3′) | |
| | | CGGAAGCAUCCUC | | | | | |
| | | GUCCACGUGACUG | | | | | |
| | | UGACGUGUGUGGC | | | | | |
| AB028669.1 | AB028669_ | CAUUUUAAGUAAG | 307 | AAGUAAGGCG | 402 | GAGCACUUCC | 497 |
| | 3440_3513 | GCGGAAGCAGCUC | | GAAGCAGCUC | | GGCUUGCCCA | |
| | | GGCGUACACAAAA | | GG(5′) | | A(3′) | |
| | | UGGCGGCGGAGCA | | | | | |
| | | CUUCCGGCUUGCC | | | | | |
| | | CAAAAUGG | | | | | |
| AB028669.1 | AB028669_ | GUCACAAGUCACG | 308 | AGUCACGUGG | 403 | CAAUCCUCUU | 498 |
| | 3548_3619 | UGGGGAGGGUUGG | | GGAGGGUUGG | | ACGUGGCCUG | |
| | | CGUUUAACCCGGA | | C(5′) | | (3′) | |
| | | AGCCAAUCCUCUU | | | | | |
| | | ACGUGGCCUGUCA | | | | | |
| | | CGUGAC | | | | | |
| AB037926.1 | AB037926_ | CGACCGCGUCCCG | 309 | CCCGAAGGCG | 404 | CGAGGUUAAG | 499 |
| | 162_232 | AAGGCGGGUACCC | | GGUACCCGAG | | GGCCAAUUCG | |
| | | GAGGUGAGUUUAC | | GU(5′) | | GGCU(3′) | |
| | | ACACCGAGGUUAA | | | | | |
| | | GGGCCAAUUCGGG | | | | | |
| | | CUUGG | | | | | |
| AB037926.1 | AB037926_ | CGCGGUAUCGUAG | 310 | UAUCGUAGCC | 405 | GGGCCCCCGC | 500 |
| | 3454_3513 | CCGACGCGGACCC | | GACGCGGACC | | GGGGCUCUCG | |
| | | CGUUUUCGGGGCC | | CCG(5′) | | GCG(3′) | |
| | | CCCGCGGGGCUCU | | | | | |
| | | CGGCGCG | | | | | |
| AB037926.1 | AB037926_ | CGCCAUUUUGUGA | 311 | AUUUUGUGAU | 406 | GCGGGGCGUG | 501 |
| | 3531_3609 | UACGCGCGUCCCC | | ACGCGCGUCC | | GCCGUAUCAG | |
| | | UCCCGGCUUCCGU | | CCUCCC(5′) | | AAAAUGG(3′) | |
| | | ACAACGUCAGGCG | | | | | |
| | | GGGCGUGGCCGUA | | | | | |
| | | UCAGAAAAUGGCG | | | | | |
| AB037926.1 | AB037926_ | GCUACGUCAUAAG | 312 | AAGUCACGUG | 407 | CCUCGGUCAC | 502 |
| | 3637_3714 | UCACGUGACUGGG | | ACUGGGCAGG | | GUGGCCUGU | |
| | | CAGGUACUAAACC | | U(5′) | | (3′) | |
| | | CGGAAGUAUCCUC | | | | | |
| | | GGUCACGUGGCCU | | | | | |
| | | GUCACGUAGUUG | | | | | |
| AB038621.1 | AB038621_ | GGCUSUGACGUCA | 313 | UGACGUCAAA | 408 | CCUCGUCACG | 503 |
| | 3511_3591 | AAGUCACGUGGGR | | GUCACGUGGG | | UGACCUGACG | |
| | | AGGGUGGCGUUAA | | RAGGGU(5′) | | UCACAG(3′) | |
| | | ACCCGGAAGUCAU | | | | | |
| | | CCUCGUCACGUGA | | | | | |
| | | CCUGACGUCACAG | | | | | |
| | | CC | | | | | |
| AB038622.1 | AB038622_ | GCCCGUCCGCGGC | 314 | GAUCGAGCGU | 409 | CCGUCCGCGG | 504 |
| | 227_293 | GAGAGCGCGAGCG | | CCCGUGGGCG | | CGAGAGCGCG | |
| | | AAGCGAGCGAUCG | | GGU(3′) | | AGCGA(5′) | |
| | | AGCGUCCCGUGGG | | | | | |
| | | CGGGUGCCGAAGG | | | | | |
| | | U | | | | | |
| AB038622.1 | AB038622_ | GGUUGUGACGUCA | 315 | UGACGUCAAA | 410 | AUCCUCGUCA | 505 |
| | 3510_3591 | AAGUCACGUGGGG | | GUCACGUGGG | | CGUGACCUGA | |
| | | AGGGCGGCGUUAA | | GAGGGCGG(5′) | | CGUCACG(3′) | |
| | | ACCCGGAAGUCAU | | | | | |
| | | CCUCGUCACGUGA | | | | | |
| | | CCUGACGUCACGG | | | | | |
| | | CC | | | | | |
| AB038623.1 | AB038623_ | GCCCGUCCGCGGC | 316 | GAUCGAGCGU | 411 | CCGUCCGCGG | 506 |
| | 228_295 | GAGAGCGCGAGCG | | CCCGUGGGCG | | CGAGAGCGCG | |
| | | AAGCGAGCGAUCG | | GGU(3′) | | AGCGA(5′) | |
| | | AGCGUCCCGUGGG | | | | | |
| | | CGGGUGCCGUAGG | | | | | |
| | | UG | | | | | |
| AB038624.1 | AB038624_ | GCCCGUCCGCGGC | 317 | GAUCGAGCGU | 412 | CCGUCCGCGG | 507 |
| | 228_295 | GAGAGCGCGAGCG | | CCCGUGGGCG | | CGAGAGCGCG | |
| | | AAGCGAGCGAUCG | | GGU(3′) | | AGCGA(5′) | |
| | | AGCGUCCCGUGGG | | | | | |
| | | CGGGUGCCGUAGG | | | | | |
| | | UG | | | | | |
| AB038624.1 | AB038624_ | GGCUGUGACGUCA | 318 | UGACGUCAAA | 413 | AUCCUCGUCA | 508 |
| | 1351_3592 | AAGUCACGUGGGG | | GUCACGUGGG | | CGUGACCUGA | |
| | | AGGGCGGCGUUAA | | GAGGGCGG(5′) | | CGUCACG(3′) | |
| | | ACCCGGAAGUCAU | | | | | |
| | | CCUCGUCACGUGA | | | | | |
| | | CCUGACGUCACGG | | | | | |
| | | CC | | | | | |
| AB041957.1 | AB041957_ | AGACCACGUGGUA | 319 | ACGUGGUAAG | 414 | CUGACCCGCG | 509 |
| | 3414_3493 | AGUCACGUGGGGG | | UCACGUGGGG | | UGACUGGUCA | |
| | | CAGCUGCUGUAAA | | GCAGCU(5′) | | CGUGA(3′) | |
| | | CCCGGAAGUAGCU | | | | | |
| | | GACCCGCGUGACU | | | | | |
| | | GGUCACGUGACCU | | | | | |
| | | G | | | | | |
| AB049608.1 | AB049608_ | CGCCAUUUUAUAA | 320 | AUUUUAUAAU | 415 | CGGGGCGUGG | 510 |
| | 3199_3277 | UACGCGCGUCCCC | | ACGCGCGUCC | | CCGUAUUAGA | |
| | | UCCCGGCUUCCGU | | CCUCC(5′) | | AAAUGG(3′) | |
| | | ACUACGUCAGGCG | | | | | |
| | | GGGCGUGGCCGUA | | | | | |
| | | UUAGAAAAUGGUG | | | | | |
| AB050448.1 | AB050448_ | UAAGUAAGGCGGA | 321 | AAGGGACAGC | 416 | AGUAAGGCGG | 511 |
| | 3393_3465 | ACCAGGCUGUCAC | | CUUCCGGCUU | | AACCAGGCUG | |
| | | CCUGUGUCAAAGG | | GC(3′) | | UCACCCUGU | |
| | | UCAAGGGACAGCC | | | | (5′) | |
| | | UUCCGGCUUGCAC | | | | | |
| | | AAAAUGG | | | | | |
| AB054647.1 | AB054647_ | UGCCUACGUCAUA | 322 | CAUAAGUCAC | 417 | UAGCUGACCC | 512 |
| | 3537_3615 | AGUCACGUGGGGA | | GUGGGGACGG | | GCGUGACUUG | |
| | | CGGCUGCUGUAAA | | CUGCU(5′) | | UCAC(3′) | |
| | | CACGGAAGUAGCU | | | | | |
| | | GACCCGCGUGACU | | | | | |
| | | UGUCACGUGAGCA | | | | | |
| AB054648.1 | AB054648_ | UUGUGUAAGGCGG | 323 | UAAGGCGGAA | 418 | GGUCAGCCUC | 513 |
| | 3439_3511 | AACAGGCUGACAC | | CAGGCUGACA | | CGCUUUGCA | |
| | | CCCGUGUCAAAGG | | CCCC(5′) | | (3′) | |
| | | UCAGGGGUCAGCC | | | | | |
| | | UCCGCUUUGCACC | | | | | |
| | | AAAUGGU | | | | | |
| AB054648.1 | AB054648_ | UACCUACGUCAUAA | 324 | UACGUCAUAA | 419 | GCUGACCCGC | 514 |
| | 3538_3617 | GUCACGUGGGAAG | | GUCACGUGGG | | GUGGCUUGUC | |
| | | AGCUGCUGUGAAC | | AAGAGCUG(5′) | | ACGUGAGU(3′) | |
| | | CUGGAAGUAGCUG | | | | | |
| | | ACCCGCGUGGCUU | | | | | |
| | | GUCACGUGAGUGC | | | | | |
| AB064595.1 | AB064595_ | UUUUCCUGGCCCG | 325 | UCGGGCGUCC | 420 | GGCCCGUCCG | 515 |
| | 116_191 | UCCGCGGCGAGAG | | CGAGGGCGGG | | CGGCGAGAGC | |
| | | CGCGAGCGAAGCG | | UG(3′) | | GCGAG(5′) | |
| | | AGCGAUCGGGCGU | | | | | |
| | | CCCGAGGGCGGGU | | | | | |
| | | GCCGGAGGUG | | | | | |
| AB064595.1 | AB064595_ | AAAGUGAGUGGGG | 326 | AAAGUGAGUG | 421 | UCCGGGUGCG | 516 |
| | 3283_3351 | CCAGACUUCGCCA | | GGGCCAGACU | | UCUGGGGGCC | |
| | | UAGGGCCUUUAAC | | UCGCC(5′) | | GCCAUUU(3′) | |
| | | UUCCGGGUGCGUC | | | | | |
| | | UGGGGGCCGCCAU | | | | | |
| | | UUU | | | | | |
| AB064595.1 | AB064595_ | GUGACGUUACUCU | 327 | CUCUCACGUG | 422 | AUCCUCGACC | 517 |
| | 3427_3500 | CACGUGAUGGGGG | | AUGGGGGCGU | | ACGUGACUGU | |
| | | CGUGCUCUAACCC | | GC(5′) | | G(3′) | |
| | | GGAAGCAUCCUCG | | | | | |
| | | ACCACGUGACUGU | | | | | |
| | | GACGUCAC | | | | | |
| AB064595.1 | AB064595_ | AGCGUCUACUACG | 328 | UCUACUACGU | 423 | AUAAACCAGA | 518 |
| | 41_116 | UACACUUCCUGGG | | ACACUUCCUG | | GGGGUGACGA | |
| | | GUGUGUCCUGCCA | | GGGUGUGU(5′) | | AUGGUAGAGU(3′) | |
| | | CUGUAUAUAAACCA | | | | | |
| | | GAGGGGUGACGAA | | | | | |
| | | UGGUAGAGU | | | | | |
| AB064596.1 | AB064596_ | GUGACGUCAAAGU | 329 | UGGCUGUUGU | 424 | CAAAGUCACG | 519 |
| | 3424_3497 | CACGUGGUGACGG | | CACGUGACUU | | UGGUGACGGC | |
| | | CCAUUUUAACCCG | | GA(3′) | | CAU(5′) | |
| | | GAAGUGGCUGUUG | | | | | |
| | | UCACGUGACUUGA | | | | | |
| | | CGUCACGG | | | | | |
| AB064597.1 | AB064597_ | GCUUUAGACGCCA | 330 | AGACGCCAUU | 425 | GUAGGCGCGU | 520 |
| | 1319_3253 | UUUUAGGCCCUCG | | UUAGGCCCUC | | UUUAAUGACG | |
| | | CGGGCACCCGUAG | | GCGG(5′) | | UCACGG(3′) | |
| | | GCGCGUUUUAAUG | | | | | |
| | | ACGUCACGGC | | | | | |
| AB064597.1 | AB064597_ | CACCCGUAGGCGC | 331 | UGUCGUGACG | 426 | UAGGCGCGUU | 521 |
| | 3221_3294 | GUUUUAAUGACGU | | UUUGAGACAC | | UUAAUGACGU | |
| | | CACGGCAGCCAUU | | GUGAU(3′) | | CACGGCAG(5′) | |
| | | UUGUCGUGACGUU | | | | | |
| | | UGAGACACGUGAU | | | | | |
| | | GGGGGCGU | | | | | |
| AB064597.1 | AB064597_ | GUCGUGACGUUUG | 332 | UGACGUUUGA | 427 | AUCCCUGGUC | 522 |
| | 3262_3342 | AGACACGUGAUGG | | GACACGUGAU | | ACGUGACUCU | |
| | | GGGCGUGCCUAAA | | GGGGGCGUGC | | GACGUCACG | |
| | | CCCGGAAGCAUCC | | (5′) | | (3′) | |
| | | CUGGUCACGUGAC | | | | | |
| | | UCUGACGUCACGG | | | | | |
| | | CG | | | | | |
| AB064598.1 | AB064598_ | CGAAAGUGAGUGG | 333 | AGUGAGUGGG | 428 | GCGUGUGGGG | 523 |
| | 3179_3256 | GGCCAGACUUCGC | | GCCAGACUUC | | GCCGCCAUUU | |
| | | CAUAAGGCCUUUA | | GC(5′) | | UAGCUU(3′) | |
| | | ACUUCCGGGUGCG | | | | | |
| | | UGUGGGGGCCGCC | | | | | |
| | | AUUUUAGCUUCG | | | | | |
| AB064598.1 | AB064598_ | CUGUGACGUCAAA | 334 | UGUGACGUCA | 429 | UCAUCCUCGU | 524 |
| | 3323_3399 | GUCACGUGGGGAG | | AAGUCACGUG | | CACGUGACCU | |
| | | GGCGGCGUGUAAC | | GGGAGGGCGG | | GACGUCACG | |
| | | CCGGAAGUCAUCC | | (5′) | | (3′) | |
| | | UCGUCACGUGACC | | | | | |
| | | UGACGUCACGG | | | | | |
| AB064598.1 | AB064598_ | CUGUCCGCCAUCU | 335 | AAAAGAGGAA | 430 | CGCCAUCUUG | 525 |
| | 3412_3485 | UGUGACUUCCUUC | | GUAUGACGUA | | UGACUUCCUU | |
| | | CGCUUUUUCAAAAA | | GCGGCGG(3′) | | CCGCUUUUU | |
| | | AAAAGAGGAAGUAU | | | | (5′) | |
| | | GACGUAGCGGCGG | | | | | |
| | | GGGGGC | | | | | |
| AB064599.1 | AB064599_ | GGUAGAGUUUUUU | 336 | AGCGAGCGGC | 431 | UAGAGUUUUU | 526 |
| | 108_175 | CCGCCCGUCCGCA | | CGAGCGACCC | | UCCGCCCGUC | |
| | | GCGAGGACGCGAG | | G(3′) | | CG(5′) | |
| | | CGCAGCGAGCGGC | | | | | |
| | | CGAGCGACCCGUG | | | | | |
| | | GG | | | | | |
| AB064599.1 | AB064599_ | GCUGUGACGUUUC | 337 | UUCAGUCACG | 432 | GUCCCUGGUC | 527 |
| | 3389_3469 | AGUCACGUGGGGA | | UGGGGAGGGA | | ACGUGAUUGU | |
| | | GGGAACGCCUAAA | | ACGC(5′) | | GAC(3′) | |
| | | CCCGGAAGCGUCC | | | | | |
| | | CUGGUCACGUGAU | | | | | |
| | | UGUGACGUCACGG | | | | | |
| | | CC | | | | | |
| AB064599.1 | AB064599_ | CCGCCAUUUUGUG | 338 | AAAAGAGGAA | 433 | CAUUUUGUGA | 528 |
| | 3483_3546 | ACUUCCUUCCGCU | | GUGUGACGUA | | CUUCCUUCCG | |
| | | UUUUCAAAAAAAAA | | GCGG(3′) | | CUUUUU(5′) | |
| | | GAGGAAGUGUGAC | | | | | |
| | | GUAGCGGCGG | | | | | |
| AB064600.1 | AB064600_ | GACUGUGACGUCA | 339 | UGUGACGUCA | 434 | UCAUCCUCGU | 529 |
| | 3378_3456 | AAGUCACGUGGGG | | AAGUCACGUG | | CACGUGACCU | |
| | | AGGGCGGCGUGUA | | GGGAGGGCGG | | GACGUCACG | |
| | | ACCCGGAAGUCAU | | (5′) | | (3′) | |
| | | CCUCGUCACGUGA | | | | | |
| | | CCUGACGUCACGG | | | | | |
| AB064600.1 | AB064600_ | CUGUCCGCCAUCU | 340 | AAAAGAGGAA | 435 | CCGCCAUCUU | 530 |
| | 9346_3542 | UGUGACUUCCUUC | | GUAUGACGUG | | GUGACUUCCU | |
| | | CGCUUUUUCAAAAA | | GCGG(3′) | | UCCGCUUUUU(5′) | |
| | | AAAAGAGGAAGUAU | | | | | |
| | | GACGUGGCGGCGG | | | | | |
| | | GGGGGC | | | | | |
| AB064601.1 | AB064601_ | GGUUGUGACGUCA | 341 | UGACGUCAAA | 436 | AUCCUCGUCA | 531 |
| | 3318_3398 | AAGUCACGUGGGG | | GUCACGUGGG | | CGUGACCUGA | |
| | | AGGGCGGCGUGUA | | GAGGGCGG(5′) | | CGUCACG(3′) | |
| | | ACCCGGAAGUCAU | | | | | |
| | | CCUCGUCACGUGA | | | | | |
| | | CCUGACGUCACGG | | | | | |
| | | CC | | | | | |
| AB064601.1 | AB064601_ | CCCGCCAUCUUGU | 342 | AAAAAAGAGG | 437 | CGCCAUCUUG | 532 |
| | 3412_3477 | GACUUCCUUCCGC | | AAGUGUGACG | | UGACUUCCUU | |
| | | UUUUUCAAAAAAAA | | UAGCGGCGG | | CCGCUUUUUC | |
| | | AGAGGAAGUGUGA | | (3′) | | (5′) | |
| | | CGUAGCGGCGGG | | | | | |
| AB064602.1 | AB064602_ | GCCCGUCCGCGGC | 343 | GAUCGAGCGU | 438 | CCGUCCGCGG | 533 |
| | 125_192 | GAGAGCGCGAGCG | | CCCGUGGGCG | | CGAGAGCGCG | |
| | | AAGCGAGCGAUCG | | GGU(3′) | | AGCGA(5′) | |
| | | AGCGUCCCGUGGG | | | | | |
| | | CGGGUGCCGUAGG | | | | | |
| | | UG | | | | | |
| AB064602.1 | AB064602_ | GACUGUGACGUCA | 344 | UGUGACGUCA | 439 | UCAUCCUCGU | 534 |
| | 3368_3446 | AAGUCACGUGGGG | | AAGUCACGUG | | CACGUGACCU | |
| | | AGGAGGGCGUGUA | | GGGAGGAGGG | | GACGUCACG | |
| | | ACCCGGAAGUCAU | | (5′) | | (3′) | |
| | | CCUCGUCACGUGA | | | | | |
| | | CCUGACGUCACGG | | | | | |
| AB064603.1 | AB064603_ | UCGCGUCUUAGUG | 345 | UUGGUCCUGA | 440 | CUUAGUGACG | 535 |
| | 3385_3447 | ACGUCACGGCAGC | | CGUCACUGUC | | UCACGGCAGC | |
| | | CAUCUUGGUCCUG | | A(3′) | | CAU(5′) | |
| | | ACGUCACUGUCAC | | | | | |
| | | GUGGGGAGGG | | | | | |
| AB064603.1 | AB064603_ | UGACGUCACUGUC | 346 | CGUCACUGUC | 441 | GUCCCUGGUC | 536 |
| | 3422_3498 | ACGUGGGGAGGGA | | ACGUGGGGAG | | ACGUGACAUG | |
| | | ACACGUGAACCCG | | GGAACAC(5′) | | ACGUC(3′) | |
| | | GAAGUGUCCCUGG | | | | | |
| | | UCACGUGACAUGA | | | | | |
| | | CGUCACGGCCG | | | | | |
| AB064604.1 | AB064604_ | CGCCAUUUUAAGU | 347 | UAAGUAAGCA | 442 | CACAGCCGGU | 537 |
| | 3436_3514 | AAGCAUGGCGGGC | | UGGCGGGCGG | | CAUGCUUGCA | |
| | | GGUGAUGUCAAAU | | UGAU(5′) | | CAAA(3′) | |
| | | GUUAAAGGUCACA | | | | | |
| | | GCCGGUCAUGCUU | | | | | |
| | | GCACAAAAUGGCG | | | | | |
| AB064605.1 | AB064605_ | CGCCAUUUUAAGU | 348 | AAGUAAGCAU | 443 | ACAGCCUGUC | 538 |
| | 3440_3518 | AAGCAUGGCGGGC | | GGCGGGCGGU | | AUGCUUGCAC | |
| | | GGUGACGUGCAAU | | GA(5′) | | AA(3′) | |
| | | GUCAAAGGUCACA | | | | | |
| | | GCCUGUCAUGCUU | | | | | |
| | | GCACAAAAUGGCG | | | | | |
| AB064606.1 | AB064606_ | CCAUCUUAAGUAG | 349 | UAAGUAGUUG | 444 | CACCAUCAGC | 539 |
| | 3377_3449 | UUGAGGCGGACGG | | AGGCGGACGG | | CACACCUACU | |
| | | UGGCGUCGGUUCA | | UGGC(5′) | | CAAA(3′) | |
| | | AAGGUCACCAUCA | | | | | |
| | | GCCACACCUACUC | | | | | |
| | | AAAAUGG | | | | | |
| AB064607.1 | AB064607_ | GCCUGUCAUGCUU | 350 | UCAUGCUUGC | 445 | CGGGUCGCCG | 540 |
| | 3502_3569 | GCACAAAAUGGCG | | ACAAAAUGGC | | CCAUAUUUGG | |
| | | GACUUCCGCUUCC | | GGACUUCCG | | UCACGUGA(3′) | |
| | | GGGUCGCCGCCAU | | (5′) | | | |
| | | AUUUGGUCACGUG | | | | | |
| | | AC | | | | | |
| AF079173.1 | AF079173_ | GCCAUUUUAAGUA | 351 | AGUAGCUGAC | 446 | CAUCCUCGGC | 541 |
| | 3475_3551 | GCUGACGUCAAGG | | GUCAAGGAUU | | GGAAGCUACA | |
| | | AUUGACGUAAAGG | | GAC(5′) | | CAA(3′) | |
| | | UUAAAGGUCAUCC | | | | | |
| | | UCGGCGGAAGCUA | | | | | |
| | | CACAAAAUGGU | | | | | |
| AF116842.1 | AF116842_ | GCCAUUUUAAGUA | 352 | AGUAGCUGAC | 447 | CAUCCUCGGC | 542 |
| | 3475_3551 | GCUGACGUCAAGG | | GUCAAGGAUU | | GGAAGCUACA | |
| | | AUUGACGUAAAGG | | GAC(5′) | | CAA(3′) | |
| | | UUAAAGGUCAUCC | | | | | |
| | | UCGGCGGAAGCUA | | | | | |
| | | CACAAAAUGGU | | | | | |
| AF116842.1 | AF116842_ | GCAUACGUCACAA | 353 | ACAAGUCACG | 448 | GGCCCCGUCA | 543 |
| | 3579_3657 | GUCACGUGGGGGG | | UGGGGGGGAC | | CGUGACUUAC | |
| | | GACCCGCUGUAAC | | CCG(5′) | | CAC(3′) | |
| | | CCGGAAGUAGGCC | | | | | |
| | | CCGUCACGUGACU | | | | | |
| | | UACCACGUGUGUA | | | | | |
| AF122913.1 | AF122913_ | GCCAUUUUAAGUA | 354 | AAGUAGCUGA | 449 | UCAUCCUCGG | 544 |
| | 3475_3551 | GCUGACGUCAAGG | | CGUCAAGGAU | | CGGAAGCUAC | |
| | | AUUGACGUGAAGG | | UGACG(5′) | | ACAA(3′) | |
| | | UUAAAGGUCAUCC | | | | | |
| | | UCGGCGGAAGCUA | | | | | |
| | | CACAAAAUGGU | | | | | |
| AF122913.1 | AF122913_ | GCACACGUCAUAA | 355 | AUAAGUCACG | 450 | GGCCCCGUCA | 545 |
| | 3579_3657 | GUCACGUGGUGGG | | UGGUGGGGAC | | CGUGAUUUGU | |
| | | GACCCGCUGUAAC | | CCG(5′) | | CAC(3′) | |
| | | CCGGAAGUAGGCC | | | | | |
| | | CCGUCACGUGAUU | | | | | |
| | | UGUCACGUGUGUA | | | | | |
| AF122914.1 | AF122914_ | GCCAUUUUAAGUC | 356 | AAGUCAGCUC | 451 | GUCAUCCUCA | 546 |
| | 3476_3552 | AGCUCUGGGGAGG | | UGGGGAGGCG | | CCAUAACUGG | |
| | | CGUGACUUCCAGU | | UGACUU(5′) | | CACAA(3′) | |
| | | UCAAAGGUCAUCC | | | | | |
| | | UCACCAUAACUGG | | | | | |
| | | CACAAAAUGGC | | | | | |
| AF122915.1 | AF122915_ | GCCAUUUUAAGUA | 357 | AGUAGCUGAC | 452 | CAUCCUCGGC | 547 |
| | 3475_3551 | GCUGACGUCAAGG | | GUCAAGGAUU | | GGAAGCUACA | |
| | | AUUGACGUAAAGG | | GAC(5′) | | CAA(3′) | |
| | | UUAAAGGUCAUCC | | | | | |
| | | UCGGCGGAAGCUA | | | | | |
| | | CACAAAAUGGU | | | | | |
| AF122915.1 | AF122915_ | GCAUACGUCACAA | 358 | CAAGUCACGU | 453 | GGCCCCGUCA | 548 |
| | 3579_3657 | GUCACGUGGAGGG | | GGAGGGGACA | | CGUGACUUAC | |
| | | GACACGCUGUAAC | | CG(5′) | | CAC(3′) | |
| | | CCGGAAGUAGGCC | | | | | |
| | | CCGUCACGUGACU | | | | | |
| | | UACCACGUGUGUA | | | | | |
| AF122916.1 | AF122916_ | GCGCCAUGUUAAG | 359 | UGUUAAGUGG | 454 | AUCCUCGACG | 549 |
| | 3458_3537 | UGGCUGUCGCCGA | | CUGUCGCCGA | | GUAACCGCAA | |
| | | GGAUUGACGUCAC | | GGAUUGA(5′) | | ACAUG(3′) | |
| | | AGUUCAAAGGUCA | | | | | |
| | | UCCUCGACGGUAA | | | | | |
| | | CCGCAAACAUGGC | | | | | |
| | | G | | | | | |
| AF122916.1 | AF122916_ | CAUGCGUCAUAAG | 360 | UAAGUCACAU | 455 | GGCCCCGACA | 550 |
| | 3565_3641 | UCACAUGACAGGG | | GACAGGGGUC | | UGUGACUCGU | |
| | | GUCCACUUAAACAC | | CA(5′) | | C(3′) | |
| | | GGAAGUAGGCCCC | | | | | |
| | | GACAUGUGACUCG | | | | | |
| | | UCACGUGUGU | | | | | |
| AF122916.1 | AF122916_ | UGGCAGCACUUCC | 361 | CGGAGAGGGA | 456 | AGCACUUCCG | 551 |
| | 91_164 | GAAUGGCUGAGUU | | GCCACGGAGG | | AAUGGCUGAG | |
| | | UUCCACGCCCGUC | | UG(3′) | | UUUUCCA(5′) | |
| | | CGCGGAGAGGGAG | | | | | |
| | | CCACGGAGGUGAU | | | | | |
| | | CCCGAACG | | | | | |
| AF122917.1 | AF122917_ | GCCAUUUUAAGUC | 362 | AAGUCAGCGC | 457 | AUCCUCACCG | 552 |
| | 3369_3447 | AGCGCUGGGGAGG | | UGGGGAGGCA | | GAACUGACAC | |
| | | CAUGACUGUAAGU | | UGA(5′) | | AA(3′) | |
| | | UCAAAGGUCAUCC | | | | | |
| | | UCACCGGAACUGA | | | | | |
| | | CACAAAAUGGCCG | | | | | |
| AF122918.1 | AF122918_ | GCCAUCUUAAGUG | 363 | UCUUAAGUGG | 458 | CAUCCUCGGC | 553 |
| | 3460_3540 | GCUGUCGCCGAGG | | CUGUCGCCGA | | GGUAACCGCA | |
| | | AUUGACGUCACAG | | GGAUUGAC(5′) | | AAGAUG(3′) | |
| | | UUCAAAGGUCAUC | | | | | |
| | | CUCGGCGGUAACC | | | | | |
| | | GCAAAGAUGGCGG | | | | | |
| | | UC | | | | | |
| AF122918.1 | AF122918_ | AUACGUCAUAAGU | 364 | AAGUCACAUG | 459 | UAGGCCCCGA | 554 |
| | 3566_3642 | CACAUGUCUAGGG | | UCUAGGGGUC | | CAUGUGACUC | |
| | | GUCCACUUAAACAC | | CACU(5′) | | GU(3′) | |
| | | GGAAGUAGGCCCC | | | | | |
| | | GACAUGUGACUCG | | | | | |
| | | UCACGUGUGU | | | | | |
| AF122919.1 | AF122919_ | CCAUUUUAAGUAA | 365 | AAGUAAGGCG | 460 | ACAGCCUUCC | 555 |
| | 3370_3447 | GGCGGAAGCAGCU | | GAAGCAGCUG | | GCUUUGCACA | |
| | | GUCCCUGUAACAA | | UCC(5′) | | A(3′) | |
| | | AAUGGCGGCGACA | | | | | |
| | | GCCUUCCGCUUUG | | | | | |
| | | CACAAAAUGGAG | | | | | |
| AF122920.1 | AF122920_ | GCCAUCUUAAGUG | 366 | AUCUUAAGUG | 461 | CAUCCUCGGC | 556 |
| | 3460_3540 | GCUGUCGCUGAGG | | GCUGUCGCUG | | GGUAACCGCA | |
| | | AUUGACGUCACAG | | AGGAUUGAC(5′) | | AAGAUGG(3′) | |
| | | UUCAAAGGUCAUC | | | | | |
| | | CUCGGCGGUAACC | | | | | |
| | | GCAAAGAUGGCGG | | | | | |
| | | UC | | | | | |
| AF122920.1 | AF122920_ | CAUACGUCAUAAG | 367 | UAAGUCACAU | 462 | UAGGCCCCGA | 557 |
| | 3565_3641 | UCACAUGACAGGA | | GACAGGAGUC | | CAUGUGACUC | |
| | | GUCCACUUAAACAC | | CACU(5′) | | GUC(3′) | |
| | | GGAAGUAGGCCCC | | | | | |
| | | GACAUGUGACUCG | | | | | |
| | | UCACGUGUGU | | | | | |
| AF122921.1 | AF122921_ | CGCCAUCUUAAGU | 368 | AAGUGGCUGU | 463 | UCCUCGGCGG | 558 |
| | 3459_3540 | GGCUGUCGCCGAG | | CGCCGAGGAU | | UAACCGCAAA | |
| | | GAUUGGCGUCACA | | UG(5′) | | (3′) | |
| | | GUUCAAAGGUCAU | | | | | |
| | | CCUCGGCGGUAAC | | | | | |
| | | CGCAAAGAUGGCG | | | | | |
| | | GU | | | | | |
| AF122921.1 | AF122921_ | CAUACGUCAUAAG | 369 | UAAGUCACAU | 464 | GGCCCCGACA | 559 |
| | 3565_3641 | UCACAUGACAGGG | | GACAGGGGUC | | UGUGACUCGU | |
| | | GUCCACUUAAACAC | | CA(5′) | | C(3′) | |
| | | GGAAGUAGGCCCC | | | | | |
| | | GACAUGUGACUCG | | | | | |
| | | UCACGUGUGU | | | | | |
| AF129887.1 | AF129887_ | GCAUACGUCACAA | 370 | ACAAGUCACG | 465 | GGCCCCGUCA | 560 |
| | 9357_3657 | GUCACGUGGGGGG | | UGGGGGGGAC | | CGUGACUUAC | |
| | | GACCCGCUGUAAC | | CCG(5′) | | CAC(3′) | |
| | | CCGGAAGUAGGCC | | | | | |
| | | CCGUCACGUGACU | | | | | |
| | | UACCACGUGGUGU | | | | | |
| AF247137.1 | AF247137_ | CCGCCAUUUUAGG | 371 | AUUUUAGGCU | 466 | UCAAACACCC | 561 |
| | 3453_3530 | CUGUUGCCGGGCG | | GUUGCCGGGC | | AGCGACACCA | |
| | | UUUGACUUCCGUG | | GUUUGACU(5′) | | AAAAAUGG(3′) | |
| | | UUAAAGGUCAAACA | | | | | |
| | | CCCAGCGACACCA | | | | | |
| | | AAAAAUGGCCG | | | | | |
| AF247137.1 | AF247137_ | CUACGUCAUAAGU | 372 | AUAAGUCACG | 467 | CCUCGCCCAC | 562 |
| | 3559_3636 | CACGUGACAGGGA | | UGACAGGGAG | | GUGACUUACC | |
| | | GGGGCGACAAACC | | GGG(5′) | | AC(3′) | |
| | | CGGAAGUCAUCCU | | | | | |
| | | CGCCCACGUGACU | | | | | |
| | | UACCACGUGGUG | | | | | |
| AF247138.1 | AF247138_ | GCCAUUUUAAGUA | 373 | AAGUAGGUGA | 468 | CCUCGGCGGA | 563 |
| | 3455_3532 | GGUGACGUCCAGG | | CGUCCAGGAC | | ACCUAUACAA | |
| | | ACUGACGUAAAGU | | U(5′) | | (3′) | |
| | | UCAAAGGUCAUCC | | | | | |
| | | UCGGCGGAACCUA | | | | | |
| | | UACAAAAUGGCG | | | | | |
| AF247138.1 | AF247138_ | CUACGUCAUAAGU | 374 | CAUAAGUCAC | 469 | GCCCCGUCAC | 564 |
| | 3561_3637 | CACGUGGGGACGG | | GUGGGGACGG | | GUGAUUUACC | |
| | | CUGUACUUAAACAC | | CUGU(5′) | | AC(3′) | |
| | | GGAAGUAGGCCCC | | | | | |
| | | GUCACGUGAUUUA | | | | | |
| | | CCACGUGGUG | | | | | |
| AF261761.1 | AF261761_ | GCCAUUUUAAGUA | 375 | UAAGUAAGGC | 470 | GCGGCGGAGC | 565 |
| | 3431_3504 | AGGCGGAAGAGCU | | GGAAGAGCUC | | ACUUCCGCUU | |
| | | CUAGCUAUACAAAA | | UAGCUA(5′) | | UGCCCAAA(3′) | |
| | | UGGCGGCGGAGCA | | | | | |
| | | CUUCCGCUUUGCC | | | | | |
| | | CAAAAUG | | | | | |
| AF351132.1 | AF351132_ | GCCAUUUUAAGUA | 376 | AGUAGCUGAC | 471 | CAUCCUCGGC | 566 |
| | 3475_3552 | GCUGACGUCAAGG | | GUCAAGGAUU | | GGAAGCUACA | |
| | | AUUGACGUAGAGG | | GAC(5′) | | CAA(3′) | |
| | | UUAAAGGUCAUCC | | | | | |
| | | UCGGCGGAAGCUA | | | | | |
| | | CACAAAAUGGUG | | | | | |
| AF351132.1 | AF351132_ | GCAUACGUCACAA | 377 | ACAAGUCACG | 472 | GGCCCCGUCA | 567 |
| | 3579_3657 | GUCACGUGGGGGG | | UGGGGGGGAC | | CGUGACUUAC | |
| | | GACCCGCUGUAAC | | CCG(5′) | | CAC(3′) | |
| | | CCGGAAGUAGGCC | | | | | |
| | | CCGUCACGUGACU | | | | | |
| | | UACCACGUGUGUA | | | | | |
| AF435014.1 | AF435014_ | GGCGCCAUUUUAA | 378 | UAAGUAAGCA | 473 | CACCGCACUU | 568 |
| | 3344_3426 | GUAAGCAUGGCGG | | UGGCGGGCGG | | CCGUGCUUGC | |
| | | GCGGCGACGUCAC | | CGAC(5′) | | ACAAA(3′) | |
| | | AUGUCAAAGGUCA | | | | | |
| | | CCGCACUUCCGUG | | | | | |
| | | CUUGCACAAAAUG | | | | | |
| | | GC | | | | | |
| AF435014.1 | AF435014_ | UGCUACGUCAUCG | 379 | AUCGAGACAC | 474 | UCGCUGACAC | 569 |
| | 3453_3526 | AGACACGUGGUGC | | GUGGUGCCAG | | ACGUGUCUUG | |
| | | CAGCAGCUGUAAA | | CAGCU(5′) | | UCAC(3′) | |
| | | CCCGGAAGUCGCU | | | | | |
| | | GACACACGUGUCU | | | | | |
| | | UGUCACGU | | | | | |
| AJ620212.1 | AJ620212_ | GCCAUUUUAAGUA | 380 | UCAUCCUCAG | 475 | CAUUUUAAGU | 570 |
| | 3360_3438 | AGCACCGCCUAGG | | CCGGAACUUA | | AAGCACCGCC | |
| | | GAUGACGUAUAAG | | CACAAAAUGG | | UAGGGAUGAC | |
| | | UUCAAAGGUCAUC | | (3′) | | (5′) | |
| | | CUCAGCCGGAACU | | | | | |
| | | UACACAAAAUGGU | | | | | |
| AJ620212.1 | AJ620212_ | ACGUCAUAUGUCA | 381 | AUAUGUCACG | 476 | GUAGGCCCCG | 571 |
| | 3470_3542 | CGUGGGGAGGCCC | | UGGGGAGGCC | | UCACGUGUCA | |
| | | UGCUGCGCAAACG | | CUGCUG(5′) | | UACCAC(3′) | |
| | | CGGAAGUAGGCCC | | | | | |
| | | CGUCACGUGUCAU | | | | | |
| | | ACCACGU | | | | | |
| AJ620218.1 | AJ620218_ | CCAUUUUAAGUAA | 382 | AAGUAAGGCG | 477 | GGCGGGGCAC | 572 |
| | 3381_3458 | GGCGGAAGCAGCU | | GAAGCAGCUC | | UUCCGGCUUG | |
| | | CCACUUUCUCACAA | | CACUUU(5′) | | CCCAA(3′) | |
| | | AAUGGCGGCGGGG | | | | | |
| | | CACUUCCGGCUUG | | | | | |
| | | CCCAAAAUGGC | | | | | |
| AJ620226.1 | AJ620226_ | CCAUUUUAAGUAA | 383 | AAGUAAGGCG | 478 | CGGCGGAGCA | 573 |
| | 3451_3523 | GGCGGAAGUUUCU | | GAAGUUUCUC | | CUUCCGGCUU | |
| | | CCACUAUACAAAAU | | CACU(5′) | | GCCCAA(3′) | |
| | | GGCGGCGGAGCAC | | | | | |
| | | UUCCGGCUUGCCC | | | | | |
| | | AAAAUG | | | | | |
| AJ620227.1 | AJ620227_ | CCAUCUUAAGUAG | 384 | UAAGUAGUUG | 479 | CACCAUCAGC | 574 |
| | 3379_3451 | UUGAGGCGGACGG | | AGGCGGACGG | | CACACCUACU | |
| | | UGGCGUGAGUUCA | | UGGC(5′) | | CAAA(3′) | |
| | | AAGGUCACCAUCA | | | | | |
| | | GCCACACCUACUC | | | | | |
| | | AAAAUGG | | | | | |
| AJ620231.1 | AJ620231_ | CGCCAUCUUAAGU | 385 | UAAGUAGUUG | 480 | ACCAUCAGCC | 575 |
| | 3429_3505 | AGUUGAGGCGGAC | | AGGCGGACGG | | ACACCUACUC | |
| | | GGUGGCGUGAGUU | | UGG(5′) | | AAA(3′) | |
| | | CAAAGGUCACCAU | | | | | |
| | | CAGCCACACCUAC | | | | | |
| | | UCAAAAUGGUG | | | | | |
| AY666122.1 | AY666122_ | UUUCGGACCUUCG | 386 | GACCUUCGGC | 481 | GACUCCGAGA | 576 |
| | 3163_3236 | GCGUCGGGGGGGU | | GUCGGGGGG | | UGCCAUUGGA | |
| | | CGGGGGCUUUACU | | GUCGGGGG(5′) | | CACUGAGG(3′) | |
| | | AAACAGACUCCGA | | | | | |
| | | GAUGCCAUUGGAC | | | | | |
| | | ACUGAGGG | | | | | |
| AY666122.1 | AY666122_ | CCAUUUUAAGUAG | 387 | AUCCUCGGCG | 482 | AGUAGGUGCC | 577 |
| | 3388_3464 | GUGCCGUCCAGCA | | GAACCUAUA(3′) | | GUCCAGCA(5′) | |
| | | CUGCUGUUCCGGG | | | | | |
| | | UUAAAGGGCAUCC | | | | | |
| | | UCGGCGGAACCUA | | | | | |
| | | UACAAAAUGGC | | | | | |
| AY666122.1 | AY666122_ | CUACGUCAUCGAU | 388 | AUCGAUGACG | 483 | AAGUAGGCCC | 578 |
| | 3494_3567 | GACGUGGGGAGGC | | UGGGGAGGCG | | CGCUACGUCA | |
| | | GUACUAUGAAACG | | UACUAU(5′) | | UCAUCAC(3′) | |
| | | CGGAAGUAGGCCC | | | | | |
| | | CGCUACGUCAUCA | | | | | |
| | | UCACGUGG | | | | | |
| AY823988.1 | AY823988_ | CCAUUUUAAGUAA | 389 | UGGCGGAGGA | 484 | AAGGCGGAAG | 579 |
| | 3452_3525 | GGCGGAAGAGCUG | | GCACUUCCGG | | AGCUGCUCUA | |
| | | CUCUAUAUACAAAA | | CUUG(3′) | | UAU(5′) | |
| | | UGGCGGAGGAGCA | | | | | |
| | | CUUCCGGCUUGCC | | | | | |
| | | CAAAAUG | | | | | |
| AY823988.1 | AY823988_ | UGCCUACGUAACA | 390 | AACAAGUCAC | 485 | CAAUCCUCCC | 580 |
| | 3554_3629 | AGUCACGUGGGGA | | GUGGGGAGGG | | ACGUGGCCUG | |
| | | GGGUUGGCGUAUA | | UUGGC(5′) | | UCAC(3′) | |
| | | ACCCGGAAGUCAA | | | | | |
| | | UCCUCCCACGUGG | | | | | |
| | | CCUGUCACGU | | | | | |
| AY823989.1 | AY823989_ | UAAGUAAGGCGGA | 391 | AGGGGUCAGC | 486 | AAGGCGGAAC | 581 |
| | 3551_3623 | ACCAGGCUGUCAC | | CUUCCGCUUU | | CAGGCUGUCA | |
| | | CCCGUGUCAAAGG | | A(3′) | | CCCCGU(5′) | |
| | | UCAGGGGUCAGCC | | | | | |
| | | UUCCGCUUUACAC | | | | | |
| | | AAAAUGG | | | | | |
| AY823989.1 | AY823989_ | UAAGUAAGGCGGA | 392 | AGGGGUCAGC | 487 | AAGGCGGAAC | 582 |
| | 3551_3623 | ACCAGGCUGUCAC | | CUUCCGCUUU | | CAGGCUGUCA | |
| | | CCCGUGUCAAAGG | | A(3′) | | CCCCGU(5′) | |
| | | UCAGGGGUCAGCC | | | | | |
| | | UUCCGCUUUACAC | | | | | |
| | | AAAAUGG | | | | | |
| DQ361268.1 | DQ361268_ | GCAGCCAUUUUAA | 393 | UAAGUCAGCU | 488 | CAUCCUCACC | 583 |
| | 3413_3494 | GUCAGCUUCGGGG | | UCGGGGAGGG | | GGAACUGGUA | |
| | | AGGGUCACGCAAA | | UCAC(5′) | | CAAA(3′) | |
| | | GUUCAAAGGUCAU | | | | | |
| | | CCUCACCGGAACU | | | | | |
| | | GGUACAAAAUGGC | | | | | |
| | | CG | | | | | |
| DQ361268.1 | DQ361268_ | UGCUACGUCAUAA | 394 | UCAUAAGUGA | 489 | UAGGCCCCGC | 584 |
| | 3519_3593 | GUGACGUAGCUGG | | CGUAGCUGGU | | CACGUCACUU | |
| | | UGUCUGCUGUAAA | | GUCUGCU(5′) | | GUCACG(3′) | |
| | | CACGGAAGUAGGC | | | | | |
| | | CCCGCCACGUCAC | | | | | |
| | | UUGUCACGU | | | | | |

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the curon may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

Therapeutic Peptides or Polypeptides

In some embodiments, the genetic element comprises a sequence that encodes a therapeutic peptide or polypeptide. Such therapeutics include, but are not limited to, small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 150 amino acids, or any range therebetween.

Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element includes a sequence encoding a protein e.g., a therapeutic protein. Some examples of therapeutic proteins may include, but are not limited to, a hormone, a cytokine, an enzyme, an antibody, a transcription factor, a receptor (e.g., a membrane receptor), a ligand, a membrane transporter, a secreted protein, a peptide, a carrier protein, a structural protein, a nuclease, or a component thereof.

In some embodiments, the composition or curon described herein includes a polypeptide linked to a ligand that is capable of targeting a specific location, tissue, or cell.

Regulatory Sequences

In some embodiments, the genetic element comprises a regulatory sequence, e.g., a promoter or an enhancer.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. In addition, one promoter element can increase an amount of products expressed for multiple DNA sequences attached in tandem. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

Replication Proteins

In some embodiments, the genetic element of the curon, e.g., synthetic curon, may include sequences that encode one or more replication proteins. In some embodiments, the curon may replicate by a rolling-circle replication method, e.g., synthesis of the leading strand and the lagging strand is uncoupled. In such embodiments, the curon comprises three elements additional elements: i) a gene encoding an initiator protein, ii) a double strand origin, and iii) a single strand origin. A rolling circle replication (RCR) protein complex comprising replication proteins binds to the leading strand and destabilizes the replication origin. The RCR complex cleaves the genome to generate a free 3′OH extremity. Cellular DNA polymerase initiates viral DNA replication from the free 3′OH extremity. After the genome has been replicated, the RCR complex closes the loop covalently. This leads to the release of a positive circular single-stranded parental DNA molecule and a circular double-stranded DNA molecule composed of the negative parental strand and the newly synthesized positive strand. The single-stranded DNA molecule can be either encapsidated or involved in a second round of replication. See for example, Virology Journal 2009, 6:60 doi: 10.1186/1743-422X-6-60.

The genetic element may comprise a sequence encoding a polymerase, e.g., RNA polymerase or a DNA polymerase.

Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the curon in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Exogenous Gene

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.). Examples of disease-associated genes and polynucleotides are listed in Tables A and B of U.S. Pat. No. 8,697,359, which are herein incorporated by reference in their entirety. Disease specific information is available from McKusick-Nathans Institute of Genetic Medicine, Johns Hopkins University (Baltimore, Md.) and National Center for Biotechnology Information, National Library of Medicine (Bethesda, Md.). Examples of signaling biochemical pathway-associated genes and polynucleotides are listed in Tables A-C of U.S. Pat. No. 8,697,359, which are herein incorporated by reference in their entirety.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

Viral Sequence

In some embodiments, the genetic element comprises at least one viral sequence. In some embodiments, the sequence has homology or identity to one or more sequence from a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., an anellovirus. Recent changes in nomenclature have classified the three anelloviruses able to infect human cells into Alphatorquevirus (TT), Betatorquevirus (TTM), and Gammatorquevirus (TTMD) Genera of the Anelloviridae family of viruses. To date anelloviruses have not been linked to any human disease. In some embodiments, the genetic element may comprise a sequence with homology or identity to a Torque Teno Virus (TT), a non-enveloped, single-stranded DNA virus with a circular, negative-sense genome. In some embodiments, the genetic element may comprise a sequence with homology or identity to a SEN virus, a Sentinel virus, a TTV-like mini virus, and a TT virus. Different types of TT viruses have been described including TT virus genotype 6, TT virus group, TTV-like virus DXL1, and TTV-like virus DXL2. In some embodiments, the genetic element may comprise a sequence with homology or identity to a smaller virus, Torque Teno-like Mini Virus (TTM), or a third virus with a genomic size in between that of TTV and TTMV, named Torque Teno-like Midi Virus (TTMD). In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein, e.g., Table 19.

TABLE 19
Examples of viral sequences, e.g., encoding capsid proteins. The first column
identifies the strain by its complete genome accession number. The second column
identifies the accession number of the protein encoded by the ORF listed in the
third column. The fourth column shows the nucleic acid sequence encoding the ORF
listed in the third column.

| | | | | SEQ ID |
|---|---|---|---|---|
| Strain # | Accession # | ORF # | Sequence | NO: |
| AF079173.1 | AAC28466.1 | ORF2 | ATGGCTGAGTTTTCCACGCCCGTCCGCAGCGGTGA | 585 |
| | | | AGCCACGGAGGGAGATCACCGCGTCCCGAGGGCG | |
| | | | GGTGCCGAAGGTGAGTTTACACACCGAAGTCAAGG | |
| | | | GGCAATTCGGGCTCGGGACTGGCCGGGCTATGGGC | |
| | | | AAGGCTCTGAAAAAAGCATGTTTATTGGCAGGCATT | |
| | | | ACAGAAAGAAAAGGGCGCTGTCACTGTGTGCTGTG | |
| | | | CGAACAACAAAGAAGGCTTGCAAACTACTAATAGTA | |
| | | | ATGTGGACCCCACCTCGCAATGATCAACAGTACCTT | |
| | | | AACTGGCAATGGTACTCAAGTGTACTTAGCCCCCAC | |
| | | | GCTGCTATGTGCGGGTGTCCCGACGCTGTCGCTCA | |
| | | | TTTTAATCATCTTGCTTCTGTGCTTCGTGCCCCGCAA | |
| | | | AACCCACCCCCTCCCGGTCCCCAGCGAAACCTGCC | |
| | | | CCTCCGACGGCTGCCGGCTCTCCCGGCTGCGCCAG | |
| | | | AGGCGCCCGGAGATAGAGCACCATGGCCTATGGCT | |
| | | | GGTGGCGCCGAAGGAGAAGACGGTGGCGCAGGTG | |
| | | | GAGACCCAGACCATGGAGGCCCCGCTGGAGGACCC | |
| | | | GAAGACGCAGACCTGCTAGACGCCGTGGCCACCGC | |
| | | | AGAAACGTAA | |
| AF129887.1 | AAD20025.1 | ORF2 | ATGGCTGGGTTTTCCACGCCCGTCCGCAGCGGTGA | 586 |
| | | | AGCCACGGAGGGAGCTCAGCGCGTCCCGAGGGCG | |
| | | | GGTGCCGAAGGTGAGTTTACACACCGCAGTCAAGG | |
| | | | GGCAATTCGGGCTCGGGACTGGCCGGGCTATGGGC | |
| | | | AAGACTCTGAAAAATGCATTTTTATCGGCAGGCATTA | |
| | | | CAGAAAGAAAAAGGCACTGTCACTGTGTGCAGTGCG | |
| | | | AGCAACACAGAAGGCTTGCAAACTTCTAAAAGTTAT | |
| | | | GTGGAGCCCTCCCCGCAACGATGAACATTACCTTAA | |
| | | | GGGACAATGGTACTCAAGTATACTTAGCTCTCACTC | |
| | | | TGCTTTCTGTGGCTGCCCCGATGCTGTCGCTCACTT | |
| | | | CAATCATCTTGCTACTGTACTTCGTGCTCCGGAAAA | |
| | | | CCCGGGACCCCCCGGGGGACATCGACCTTCTCCGC | |
| | | | TCCGGGTCCTACCCGCTCTCCCGGCTGCTCCCGAG | |
| | | | GCGCCCGGTGATCGAGCGCCATGGCCTATGGGTTG | |
| | | | TGGAGGAGACGGCGAAGGAGGTGGAAGAGGTGGA | |
| | | | GACGCAGACGGTGGAGACGCCGCTGGAGGACCCG | |
| | | | CCGACGCAGACCTGCTGGACGCCGTAGACGCCGCA | |
| | | | GAACAGTAA | |
| AF116842.1 | AAD29635.1 | ORF2 | ATGGCTGAGTTTTCCACGCCCGTCCGCAGCGGTGA | 587 |
| | | | AGCCACGGAGGGAGATTACCGCGTCCCGAGGGCG | |
| | | | GGTGCCGAAGGTGAGTTTACACACCGAAGTCAAGG | |
| | | | GGCAATTCGGGCTCGGGACTGGCCGGGCTATGGGC | |
| | | | AAGGCTCTGAAAAAAGCATGTTTATTGGCAGGCATT | |
| | | | ACAGAAAGAAAAGGGCGCTGTCACTGTGTGCTGTG | |
| | | | CGAACAACAAAGAAGGCTTGCAAACTACTAATAGTA | |
| | | | ATGTGGACCCCACCTCGCAATGATCAACAGTACCTT | |
| | | | AACTGGCAATGGTACTCAAGTGTACTTAACCCCCAC | |
| | | | GCTGCTATGTTCGGGTGTCCCGACGCTGTCGCTCAT | |
| | | | TTTAATCATCTTGCTTCTGTGCTTCGTGCCCCGCAAA | |
| | | | ACCCACCCCCTCCCGGTCCCCAGCGAAACCTGCCC | |
| | | | CTCCGACGGGTGCCGGCTCTCCCGGCTGCGCCAGA | |
| | | | GGCGCCCGGAGATAGAGCACCATGGCCTATGGCTT | |
| | | | GTGGCACCGAAGGAGAAGACGGTGGCGCAGGTGG | |
| | | | AAACGCACACCATGGAAGCGCCGCTGGAGGACCCG | |
| | | | AAGACGCAGACCTGCTAGACGCCGTGGCCGCCGCA | |
| | | | GAAACGTAA | |
| AB026345.1 | BAA85661.1 | ORF2 | ATGTTTATTGGCAGGCATTACAGAAAGAAAAGGGCG | 588 |
| | | | CTGTCACTGTGTGCTGTGCGAACAACAAAGAAGGCT | |
| | | | TGCAAACTACTAATAGTAATGTGGACCCCACCTCGC | |
| | | | AATGATCAACAGTACCTTAACTGGCAATGGTACTCAA | |
| | | | GTGTACTTAGCTCCCACGCTGCTATGTGCGGGTGTC | |
| | | | CCGACGCTGTCGCTCATTTTAATCATCTTGCTTCTGT | |
| | | | GCTTCGTGCCCCGCAAAACCCACCCCCTCCCGGTC | |
| | | | CCCAGCGAAACCTGCCCCTCCGACGGCTGCCGGCT | |
| | | | CTCCCGGCTGCGCCAGAGGCGCCCGGAGATAGAG | |
| | | | CACCATGGCCTATGGCTGGTGGCGCCGAAGGAGAA | |
| | | | GACGGTGGCGCAGGTGGAGACGCAGACCATGGAG | |
| | | | GCGCCGCTGGAGGACCCGAAGACGCAGACCTGCTA | |
| | | | GACGCCGTGGCCGCCGCAGAAACGTAA | |
| AB026346.1 | BAA85663.1 | ORF2 | ATGTTTATTGGCAGGCATTACAGAAAGAAAAGGGCG | 589 |
| | | | CTGTCACTGTGTGCTGTGCGAACAACAAAGAAGGCT | |
| | | | TGCAAACTACTAATACTAATGTGGACCCCACCTCGC | |
| | | | AATGACCAACAGTACCTTAACTGGCAATGGTACTCA | |
| | | | AGTATACTTAGCTCCCACGCTGCTATGTGCGGGTGT | |
| | | | CCCGACGCTGTCGCTCATTTTAATCATCTTGCGTCT | |
| | | | GTGCTTCGTGCCCCGCAAAACCCACCCCCTCCCGG | |
| | | | TCCCCAGCGAAACCTGCCCCTCCGACGGCTGCCGG | |
| | | | CTCTCCCGGCTGCGCCAGAGGCGCCCGGAGATAGA | |
| | | | GCACCATGGCCTATGGCTGGTGGCGCCGAAGGAGA | |
| | | | AGACGGTGGCGCAGGTGGAGACGCAGACCATGGA | |
| | | | GGCGCCGCTGGAGGACCCGAAGACGCAGACCTGCT | |
| | | | AGACGCCGTGGCCGCCGCAGAAACGTAA | |
| AB026347.1 | BAA85665.1 | ORF2 | ATGTTTATTGGCAGGCATTACAGAAAGAAAAGGGCG | 590 |
| | | | CTGTCACTGTGTGCTGTGCGAACAACAAAGAAGGCT | |
| | | | TGCAAACTACTAATACTAATGTGGACCCCACCTCGC | |
| | | | AATGACCAACAGTACCTTAACTGGCAATGGTACTCA | |
| | | | AGTATACTTAGCTCCCACGCTGCTATGTGCGGGTGT | |
| | | | CCCGACGCTGTCGCTCATTTTAATCATCTTGCTTCTG | |
| | | | TGCTTCGTGCCCCGCAAAACCCACCCCCTCCCGGT | |
| | | | CCCCAGCGAAACCTGCCCCTCCGACGGCTGCCGGC | |
| | | | TCTCCCGGCTGCGCCAGAGGCGCCCGGAGATAGAG | |
| | | | CGCCATGGCCTATGGCTGGTGGCGCCGAAGGAGAA | |
| | | | GACGGTGGCGCAGGTGGAGACGCAGACCATGGAG | |
| | | | GCGCCGCTGGAGGACCCGAAGACGCAGACCTGCTA | |
| | | | GACGCCGTGGCCGCCGCAGAAACGTAA | |
| AB038622.1 | BAA93585.1 | ORF2 | ATGCCGTGGAGACCGCCGGTACATAACGTTCCAGG | 591 |
| | | | TCGCGAAAATCAATGGTTTGCAGCGTTTTTTCACTCG | |
| | | | CATGCTTCTTTCTGCGGCTGTGGTGACCCTGTTGGG | |
| | | | CATATTAACAGCATTGCTCCTCGCTTTCCTAACGCC | |
| | | | GGTCCACCGAGACCACCTCCAGGGCTAGAGCAGCA | |
| | | | GAACCCCGAGGGCCCGACGGGTCCCGGAGGTCCC | |
| | | | CCCGCCATCTTGCCAGCTCTGCCGGCCCCGGCAGA | |
| | | | CCCTGAACCGCCGCCACGGCTTGGTGGTGGGGCAG | |
| | | | ATGGAGGCGCCGCTGGAGGCCTCGCTATCGCAGAC | |
| | | | GCACCTGGAGGGTACGAAGAAGACGACCTAGACGA | |
| | | | ACTTTTCGCCGCCGCCGCCGAGGACGATATGTGA | |
| AB038623.1 | BAA93588.1 | ORF2 | ATGCCGTGGAGACCGCCGGCACATAACGTTCCGGG | 592 |
| | | | TAGGGAAAATCAATGGTTCGCAGCTGTGTTTCACTC | |
| | | | GCATGCTTCTTGGTGCGGCTGTGGTGACGTTGTTGG | |
| | | | GCATCTTAATACCATTGCTACTCGCTTTCCTAACGCC | |
| | | | GGTCCCCCGAGACCACCTCCAGGGCTAGACCAGCA | |
| | | | GAACCCCGAGGGCCCGGCGGGTCCCGGAGGTCCC | |
| | | | CCCGCCATCTTGCCTGCTCTGCCGGCCCCGGCAGA | |
| | | | CCCTGAACCGCCGCCACGGCGTGGTGGTGGGGCA | |
| | | | GATGGAGGCGTCGATGGAGGCCTCGCTATCGCAAA | |
| | | | CGCACCTGGAGATTACGGAGACGACGACCTAGACG | |
| | | | AACTTTTCGCCGCCGCCGCCGAAGACAATATGTGA | |
| AB038624.1 | BAA93591.1 | ORF2 | ATGCCGTGGAAACCGCCGCGACATAACGTTCCGGG | 593 |
| | | | TAGGGAAAACCAATGGTTTGCAGCAGTGTTTCACTC | |
| | | | GCATGCTTCTTGGTGCGGCTGTGCTGACGTTGTTGG | |
| | | | CCATCTTAATAGCATTGCTACTCGCTTTCCTAACATC | |
| | | | GGTCCCCCGAGACCACCTCCAGGGCTAGACCAGCA | |
| | | | GAACCCCGAGGGCCCGGCGGGTCCCGGAGGTCCC | |
| | | | CCCGCCATCTTGCCTGCTCTGCCGGCCCCGGCAAA | |
| | | | CCCTGAACCGCCGCCACGGCGTGGTGGTGGGGCA | |
| | | | GATGGAGGCGCCGCTGGAGGCCTCGCTATCGCAGA | |
| | | | CGCACCTGGAGGGTACGCAGAAGACGACCTAGACG | |
| | | | AACTTTTCGCCGCCGCCGCCGAGGACGATATGTGA | |
| AF254410.1 | AAF71534.1 | ORF2 | ATGTTTCCTGGTAGGATCCACAGAAAGAAAAGGAAA | 594 |
| | | | GTGCTATTGTCCCCACTGCACCCTGCACCGAAAACT | |
| | | | CGCCGGGTTATGAGCTGGTCTCGTCCAATACACGAT | |
| | | | GCCCCAGCCATTGAGCGTAACTGGTGGGAATCCAC | |
| | | | AGCTCGATCCCACGCATGTTGCTGTGGCTGCGGTAA | |
| | | | TTTTGTTAATCATATTAATGTACTGGCTAATCGGTAT | |
| | | | GGCTTTACTGGCTCCGCGCACACGCCGGGTGGTCC | |
| | | | CCGGCCGAGGCCCCCGACAGTGAGCTCTGGTCCCA | |
| | | | GTACTTCCTACCGACACCCCGAGACCGGCTTTACCA | |
| | | | TGGCATGGGGATACTGGTGGAGAAGGCGCTTCTGC | |
| | | | GACCGAGGAGACGCTGGAAGAAGGTGGCGGCGCC | |
| | | | GCCGAGACTACAACCCAGAAGATCTCGACGCTCTGT | |
| | | | TCGACGCCCTCGACGAAGAGTAA | |
| AB050448.1 | BAB19927.1 | ORF2 | ATGAGCTTTGTAGAACCCTTACTAACCAGCACCCAC | 595 |
| | | | AGAGAGATAGCATACTACCATGGCTGTGTTCAGATG | |
| | | | CACAAAGCCTTCTGTGGGTGTGACAACTTTCTTACC | |
| | | | CACCTGCAACGCATAACAACATACATCTCTGCTAAC | |
| | | | CAACACACTCCACCCAGCACACCCTCAAACACCCTC | |
| | | | CGTAGAGCCCGGGCCCTGCCCGCGGCTCCGGAGC | |
| | | | CAGCTCCATGGCGTGGACCTGGTGGTGGCAGAGGA | |
| | | | GGCGCCGAAGGTGGCCGTGGAGAAGGAGAAGGTG | |
| | | | GAGAAGACTACGCACAAGAAGACCTAGACGCCTTGT | |
| | | | TCGACGCCGTCGCAAGAGATACAGAGTAA | |
| AY026465.1 | AAK01941.1 | ORF2 | ATGCACTTTTCTCGAATAAACAGAAAGAAAAAGAAAG | 596 |
| | | | TGCTACTGCTTTGCGTGCCAGCAGCTAAGAAAAAAC | |
| | | | CAACTGCTATGAGCTTCTGGAGACCTCCGGTGCACA | |
| | | | ATGTCACGGGGATCCAGCGCCTGTGGTACGAGTCC | |
| | | | TTTCACCGTGGCCATGCTGCTTTTTGTGGTTGTGGG | |
| | | | GATCCTATACTTCACATTACTACACTTGCTGAGACAT | |
| | | | ATGGCCATCCAACAGGCCCGAGACCTTCTGGGTCAT | |
| | | | CGGGAGTAGACCCCGGCCCCAATATCCGTCGAGCC | |
| | | | AGGCCTGCCCCGGCCGCTCCGGAGCCCTCACAGGT | |
| | | | TGATTCCAGACCGGCCCTGCCATGGCATGGGGATG | |
| | | | GTGGAAGCGACGGCGGCGCTGGTGGTTCCGGAAG | |
| | | | CGGTGGACCCGTGGCAGACTTCGCAGACGATGGCC | |
| | | | TAGACCAGCTCGTCGGCGCCCTAGACGACGAAGAG | |
| | | | TAA | |
| AY026466.1 | AAK01943.1 | ORF2 | ATGCACTTTTCTAGGATACAAAGAAAGAAAAGGCTAT | 597 |
| | | | TGCTACTGCAGACACTGCCAGCTTCAAAGAAAACTA | |
| | | | GGCAACTTCTGAGAGGTATGTGGAGCCCACCCACA | |
| | | | GACGATGAACGTGTCCGTGAGCGTAAATGGCTCCTC | |
| | | | TCAGTTTTTCAGTCTCACTGTGCTTTCTGTGGCTGCA | |
| | | | ATGATCCTATCGGTCACCTTTGTCGCTTGGCTACTCT | |
| | | | GTCTAACCGCCCGGAGAGCCCGGGGCCCTCCGGA | |
| | | | GGACCCCGTACTCCTCAGATCCGGCACCTACCCGC | |
| | | | TCTCCCGGCTGCTCCCCAAGAGCCCGGTGATCGAG | |
| | | | CACCATGGCCTATGGCTGGTGGGCCCGGAGACGGA | |
| | | | GACGCTGGCGCCGCTGGAAGCGCAGGCCCTGGAG | |
| | | | ACGCCGATGGAGGACCCGCAGACGCAGACCTCGTC | |
| | | | GCCGCTATAGACGCCGCAGACATGTAA | |
| AF345521.1 | AAK11697.1 | Orf2 | ATGCACTTTCGCAGAGTCTCAGCGAAAAGGAAACTG | 598 |
| | | | CTACTGCTTCCTCTGCACCCTGCATCGCAGACACCT | |
| | | | GCCATGAGCTTCAGGGCGCCCTCTCTTAATGCCGGT | |
| | | | CAACGAGAGCAGCTATGGTTCGAGTCCATCGTCCGA | |
| | | | TCCCATGACAGTTATTGCGGGTGTGGTGATACTGTC | |
| | | | GCTCATTTTAATAACATTGCTACTCGCTTTAACTATCT | |
| | | | GCCTGTTACCTCCTCGCCTCTGGATCCTTCCTCGGG | |
| | | | CCCGCCGCGAGGCCGTCCAGCGCTCCGCGCACTC | |
| | | | CCGGCTCTGCCAGCGGCACCCTCCACCCCCTCTAC | |
| | | | TAGCCGACCATGGCGTGGTGGGGCAGATGGAGAAG | |
| | | | GTGGCCGCGGCGCCGGTGGAGGAGATGGCGGCGC | |
| | | | CGCCGTAGAAGGAGACTACCAACAAGAAGAACTCG | |
| | | | ACGAGCTGTTCGCGGCCTTGGAAGACGACCAAGAA | |
| | | | AGACGGTAA | |
| AF345522.1 | AAK11699.1 | Orf2 | ATGTTTCTTGGCAGGGCCTGGAGAAAGAAAAGGCAA | 599 |
| | | | GTGCCACTGCCGACACTGCCAGTGGTGCCGCTTCC | |
| | | | ACAACCTTCACCTATGAGCAGCCAGTGGAGACCCCC | |
| | | | GGTTCACAATGTCCAGGGGCTGGAGCGCAATTGGT | |
| | | | GGGAGTGCTTCTTCCGTTCTCATGCTTGTTTTTGTG | |
| | | | GCTGTGGTGATGCTATTACTCATATTAATCATCTGGC | |
| | | | GACTCGTTTTGGACGTCCTCCTACTACCTCAACTCC | |
| | | | CCGAGGACCGCAGGCACCTCCAGTGACTCCGTACC | |
| | | | CGGCCCTGCCGGCCCCAGAGCCTAGCCCTGAGCCA | |
| | | | TGGCGTGGCGCCGGTGGCGATGGCGGCCGTGGTG | |
| | | | GAGACGCCGGAGGCGCCGCCGGTGGAGAAGGAGA | |
| | | | CGGAGGAGACCCAGACGACGCCGCCCTTATCGACG | |
| | | | CCGTCGACCTCGCAGAGTAA | |
| AF345525.1 | AAK11705.1 | Orf2 | ATGTTTCTTGGTAAAATTTACAGACAGAAAAGGAAAG | 600 |
| | | | TGCCACTGTACGGCCTGCCAGCTCCAAAGAAAAAAC | |
| | | | CACCTACTGCTATGAGCCACTGGAGCAGACCCGTC | |
| | | | CACCATGCAACGGGGATCGAGCACCTCTGGTACCA | |
| | | | GTCTGTTATTAACAGCCATTCTGCTAGCTGCGGTTG | |
| | | | TGGCGATCCTGTACGCCACTTTACTTATCTTGCTGA | |
| | | | GAGGTATGGCTTTGCCCCAACTTCCCGGGCCCCGC | |
| | | | CGGTAGCCCCAACGCCCACCATCCGTAGAGCCAGG | |
| | | | CCCGCGCCTGCCGCTCCGGAGCCCCGTGCCCTACC | |
| | | | ATGGCATGGGGATGGTGGAGACGAAGGCGCAAGTG | |
| | | | GTGGTGGAGACGCCGGTTCGCCCGAAGCAGACTTC | |
| | | | GCAGACGACGGATTAGACGCCCTCGTCGCCGCACT | |
| | | | CGACGAAGAACAGTAA | |
| AF345527.1 | AAK11709.1 | Orf2 | ATGTTTCTCGGCAGGCCTTACAGAAAGAAGAGGCAA | 601 |
| | | | GTGCCACTGCCTGGCGTGCACCATCCACCGCACCC | |
| | | | ACGGCCTAGCATGAGCCACCACTGGCGGGAGCCCA | |
| | | | TCGACAATGTCCCCAACCGGGAGAGGCACTGGCTC | |
| | | | GGGTCCGTCCTCCGAGGCCACCGAGCTTTTTGTGG | |
| | | | TTGTCGGGATCCTGTGCTTCATTTTACTAATCTGGTT | |
| | | | GCACGTTACAATCTTCAGGGCGGTGGTCCCTCAGC | |
| | | | GGGTAGTCTTAGGGATCCGCCGCCACTGAGGAGGG | |
| | | | CGCTGCCGCCACCGCCGTCCCCCCGACCGCCATGT | |
| | | | CCTGGTGGGGATGGCGCCGCCGATGGTGGTGGAA | |
| | | | GCCACGGAGGCGATGGAGACGCAGGAGGGCGCGC | |
| | | | CGCCCGAGACGACTACCGCGACGACGATATAGAAG | |
| | | | ACCTACTCGCCGCTATCGAGGCAGACGAGTAA | |
| AF345528.1 | AAK11711.1 | Orf2 | ATGCGATTTTCTCGAATTTATCGCAGAAAGAAGAGG | 602 |
| | | | CTACTGCCACTGCTACTGGTGCCAACAGAACCGAAA | |
| | | | GAACAATTTGTGATGAGCTGGCGCTGTCCCTTAGAA | |
| | | | AATGCCTATAAGAGGGAAATTAACTTCCTCAGAGGG | |
| | | | TGCCAAATGCTTCACACTTGTTTTTGTGGTTGTGATG | |
| | | | ATTTTATTAATCATATTATTCGCCTACAAAATCTTCAC | |
| | | | GGGAATTTACACCAACCCACCGGCCCGTCCACACCT | |
| | | | CCAGTAGGCCGTAGAGCTCTGGCCCTGCCGGCAGC | |
| | | | TCCGGAACCATGGCGTGGAGATGGTGGTGGGCCCG | |
| | | | AAGGCGACCGAACCGCCGATGGACCCGCAGACGCT | |
| | | | GGAGGAGACTACGCACCCGGAGACCTAGACGACCT | |
| | | | GTTCGCCGCCGCCGCCGCCGACCAAGAGTAA | |
| AF345529.1 | AAK11713.1 | Orf2 | ATGGGCAACGCTCTTAGGGTATTCATTCTTAAAATGT | 603 |
| | | | TTATCGGCAGGGCCTACCGCCACAAGAAAAGGAAA | |
| | | | GTGCTACTGTCCGCACTGCGAGCTCCACAGGCGTC | |
| | | | TCGGAGGGCTATGAGTTGGAGACCCCCTGTACACG | |
| | | | ATGCGCCCGGCATCGAGCGCAATTGGTACGAGGCC | |
| | | | TGTTTCAGAGCCCACGCTGGAACTTGTGGCTGTGGC | |
| | | | AATTTTATTATGCACATTAATCTTCTGGCTGGGCGTT | |
| | | | ATGGTTTTACTCCGGTATCAGCACCACCAGGTGGTC | |
| | | | CTCCTCCGGGCACCCCGCAGATAAGGAGAGCCAGA | |
| | | | CCTAGTCCCGCCGCGCCCGAACAGCCCCAGGCCCT | |
| | | | ACCATGGCATGGGGATGGTGGAGACGGTGGCGCC | |
| | | | GGTGGCCCACCAGACGCTGGAGGAGACGCCGTCG | |
| | | | CCGGCGCCCCGTACGGAGAACAAGAGCTCGCCGAC | |
| | | | CTGCTCGACGCTATAGAAGACGACGAACAGTAA | |
| AF371370.1 | AAK54732.1 | ORF2 | ATGGCACACCCGGGCATGATGATGCTAAGCAAAATG | 604 |
| | | | AAAATACTAGTACCCAGTTCTGACACCAGACCGGGG | |
| | | | GGCAGACGCAGAGTAAAAGTTAAAATAAGACCCCCG | |
| | | | GCCCTTTTAGAAGACAAGTGGTACACTCAGCAAGAT | |
| | | | CTAGCGCCCGTTAATCTTGTGTCACTTGTGGTTTCT | |
| | | | GCGACTAGCTTCATACATCCGTTTAGCCAACCACAA | |
| | | | ACGAACAACATTTGCACAACTTTTCAGGTGTTGAAAG | |
| | | | ACATGTACTATGACTGCATAGGAGTTAGTTCCACTTT | |
| | | | AGACGACAAATATAAAAAATTATTTCAAAAATTATACA | |
| | | | CTAAATGCTGCTACTTTGAAACATTTCAAACAATAGC | |
| | | | CCAGCTAAACCCCGGCTTTAAATCTGCTAAAAAAACT | |
| | | | ACAACTGGCTCCGGTAAGGAAGCTGCCACACTAGG | |
| | | | CGACGCAGTTACACAATTAAAAAACCAACACGGTAG | |
| | | | TTTTTATACTGGAAACAATAGTACTTTTGGCTGCTGT | |
| | | | ACATATAACCCCACTGAAGAAATAGGTAAAGCAGCA | |
| | | | AATGAGTGGTTCTGGAACCAATTAACTGCAACAGAG | |
| | | | TCAGACACACTAGGACAGTACGGACGTGCCTCAATT | |
| | | | AAGTACTTTGAATATCACACAGGACTATACAGTTCCA | |
| | | | TATTTTTAAGTCCACTAAGGAGCAACCTAGAATTTTC | |
| | | | TACAGCATACCAGGATGTAACATACAATCCACTGAC | |
| | | | AGACCTAGGCATAGGCAACAGAATCTGGTACCAATA | |
| | | | CAGTACCAAGCCAGACACTACATTTAACGAAACACA | |
| | | | GTGCAAATGTGTACTAACTGACCTGCCCCTGTGGTC | |
| | | | CCTGTTTTATGGATACGTAGACTTTATAGAGTCAGAG | |
| | | | CTAGGCATAAGCGCAGAGATACACAACTTTGGCATA | |
| | | | GTTTGCGTTCAGTGCCCATACACCTTTCCACCCATG | |
| | | | TTCGACAAGTCTAAGCCAGACAAGGGCTACGTATTT | |
| | | | TATGACACCCTTTTTGGTAACGGAAAGATGCCAGAC | |
| | | | GGTTCCGGACACGTACCTACCTACTGGCAGCAGAG | |
| | | | ATGGTGGCCAAGATTTAGCTTCCAGAGACAAGTAAT | |
| | | | GCATGACATTATTCTGACTGGACCTTTTAGTTACAAA | |
| | | | GATGACTCTGTAATGACTGGACTAACAGCAGGCTAC | |
| | | | AAGTTTAAATTCACATGGGGCGGTGATATGATCTCC | |
| | | | GAACAGGTCATTAAAAACCCCGACAGAGGTGACGG | |
| | | | ACGCGAATCCTCCTATCCCGATAGACAGCGCCGCG | |
| | | | ACCTACAAGTTGTTGACCCTCGCTCCATGGGGCCCC | |
| | | | AATGGGTATTCCACACCTTTGACTACAGGAGGGGAC | |
| | | | TATTTGGAAAGGACGCTATTAAACGAGTGTCAGAAA | |
| | | | AACCGACAGATCCTGACTACTTTACAACACCTTACAA | |
| | | | AAAACCGAGGTTTTTCCCCCCAACAGCAGGAGAAGA | |
| | | | AAGACTGCAAGAAGAAAACTACACTTTACAGGAGAA | |
| | | | AAGAGACCCGTTCTCGTCAGAAGAGGGGCCGCAGA | |
| | | | GGACGCAAGTCCTCCAGCAGCAGGTCCTCCAGTCG | |
| | | | GAGCTCCAGCAGCAGCAGGAGCTCGGGGACCAGCT | |
| | | | CAGATTCCTCCTCAGGGAAATGTTCAAAACCCAAGC | |
| | | | GGGTATACACATGAACCCCCGCGCATTTCAAGAGCT | |
| | | | GTAA | |
| AB060596.1 | BAB69915.1 | ORF2 | ATGAGCTGGTGTACTCCAGTTGAAAATGCCTATAAG | 605 |
| | | | AGAGAGATCCACTTTCTCAGGGGCTGTCAACTGCTT | |
| | | | CACACTAGCTTTTGTGGTTGCGATGATTTTATTAATC | |
| | | | ATATTATTCGCCTACAAAATCTTCACGGCAACCTACA | |
| | | | CCAGCCCACGGGACCGTCCACACCTCCAGTGACCC | |
| | | | GTAGAGCTCTGGCCTTGCCGGCTGCTCCGGAGTCA | |
| | | | TGGCGTTCCGGTGGTGGTGGTGGAGACGCCGCCC | |
| | | | GCAGCGACGATGGACCCGGCGCCGATGGAGGAGA | |
| | | | CTACGAACCCGCCGACCTAGACGCACTGTACGACG | |
| | | | CCGTCGCCGCAGACCAAGAGTAA | |
| AB060592.1 | BAB69899.1 | ORF2 | ATGAGCTTTGTAGAACCGTTACTAAGCAGCACCCAC | 606 |
| | | | CGAGAGATAGCATTCTACCATGGCTGTGTTCAAATG | |
| | | | CACAAGGCCTTCTGTGGCTGTGACAACTTTCTTACC | |
| | | | CACCTGCAGCGCATAACAACATACATCTCTGCTAAT | |
| | | | CAACACACTCCACCCAGCACACCCTCAAACACCCTC | |
| | | | CGTAGAGCCCGGGCCCTGCCCGCGGCTCCGGAGC | |
| | | | CAGCTCCATGGCGTGGACCTGGTGGTGGCAGAGGA | |
| | | | GGCGCCGAAGGTGGCCGTGGAGAAGGAGAAGGTG | |
| | | | GAGAAGACTACGCACCAGAAGACCTAGACGACTTGT | |
| | | | TCGCCGCCGTCGCAAGAGATACAGAGTAA | |
| AB060593.1 | BAB69903.1 | ORF2 | ATGAGTCTGTGGCGACCCCCGGTCCACAATGCCCC | 607 |
| | | | CGGCAGAGAGAGACTTTGGTTTCAGGCCTGTTACGA | |
| | | | ATCTCACAGTGCTTTTTGTGGCTGTGGTAGCTTTATT | |
| | | | CTTCATCTTACTAGCTTGGCTGCACGTTTTAATTTTC | |
| | | | AGGCCGGGCCACCGCCTCCCGGGGGTCCCCGGGC | |
| | | | GGAGACCCCGCCGATTCTGAGGGCGCTGCCGGCAC | |
| | | | CCCAGCCGCGCCGCCACCGCCAGACGGAGAACCC | |
| | | | CGGGTCTGAGCCATGGCCTGGAGATGGTGGTGGAG | |
| | | | ACGGCGCTGGAAGCCAAGAAGGCGGCCAGCGTGG | |
| | | | ACCAAGTACCGCAGACGCAGGTGGAGACGACTTCG | |
| | | | ACCCCGCAGACCTAGAAGACTTGCTCGCGGCCGTC | |
| | | | GAAGAAGACGAACAGTAA | |
| AB060595.1 | BAB69911.1 | ORF2 | ATGAATCTCTGGCGACCCCCTCTGAGAAATATCCCC | 608 |
| | | | CACAGGGAGAGATGTTGGCTTGAGGCCTGTCTCAG | |
| | | | AGCCCACGATTCTTTTTGTGGCTGTCCTAGTCCTATT | |
| | | | GTTCATTTTTCTAGTCTGGTTGCACGTTTTAATCTAC | |
| | | | AAGGAGGCCCGCCGCCAGAGGATGACTCCCCACAG | |
| | | | GGCGCGCCAGTCCTGAGGGCCCTGCCGGCACCGA | |
| | | | GCCCCCACAGGCACACCCGCACGGAGAACCCCTCC | |
| | | | GGTGAGCCATGGCCTACTCCTACTGGTGGCGCCGC | |
| | | | CGGAGGTGGCCGTGGAGAGGCCGATGGAGGCGCT | |
| | | | GGAGGCGCCGCAGACGAATACCGCGCCGAAGACCT | |
| | | | AGACGACCTGTTCGCCGCTATCGAAGGAGACCAGT | |
| | | | AA | |
| AB064596.1 | BAB79313.1 | ORF2 | ATGCCGTGGAGACCGCCGGCTCATAACGTCCAGGG | 609 |
| | | | GCGAGAGAGCCAGTGGTTCGCGGCTTGTTTTCACG | |
| | | | GCCACGCTTCGTTTTGCGGCTGCGGTGACTTTATTG | |
| | | | GGCATATTAACAGCCTTGCTCCTCGCTTTCCTAACAA | |
| | | | CCAAGGACCCCCGCATCCACCTGCCTTAAACAGGC | |
| | | | CACCTGCACAGGGCCCAGAAAGCCCCGGGGGTTCC | |
| | | | ATACTACCCCTGCCAGCCCTACCGGCACCACCTGAT | |
| | | | CCGCCACCACGGCCTGGTGGTGGGGAAGACGGTG | |
| | | | GCGACGCCGCCCGTGGGGCCGCTGGCGCCGCCGA | |
| | | | AGGCGCGTATGGAGAAGAAGACCTAGAACTGCTGTT | |
| | | | CGCCGCCGCCGAGGAAGACGATATGTGA | |
| AB064597.1 | BAB79317.1 | ORF2 | ATGCCGTGGAGACCGCCGGTGCATAGTGTCCAGGG | 610 |
| | | | GCGAGAGGATCAGTGGTTCGCGAGCTTTTTTCACGG | |
| | | | CCACGCTTCATTTTGCGGTTGCGGTGACGCTGTTGG | |
| | | | CCATCTTAATAGCATTGCTCCTCGCTTTCCTCGCGC | |
| | | | CGGTCCACCAAGGCCCCCTCCGGGGCTAGAGCAGC | |
| | | | CTAACCCCCCGCAGCAGGGCCCGGCCGGGCCCGG | |
| | | | AGGGCCGCCCGCCATCTTGGCGCTGCCGGCTCCGC | |
| | | | CCGCGGAGCCTGACGACCCGCAGCCACGGCGTGG | |
| | | | TGGTGGGGACGGTGGCGCCGCCGCTGGCGCCGCA | |
| | | | GGCGACCGTGGAGACCGAGACTACGACGAAGAAGA | |
| | | | GCTAGACGAGCTTTTCCGCGCCGCCGCCGAAGACG | |
| | | | ATTTGTAA | |
| AB064599.1 | BAB79325.1 | ORF2 | ATGCCGTGGTCTCTGCCGAGACATAATATCAGAACG | 611 |
| | | | AGAGAAGATCTCTGGGTGCAATCGATTCTTTATTCAC | |
| | | | ATGACACTTTTTGTGGCTGTGATAATATTCCTGAGCA | |
| | | | TCTTACTGGCCTCCTGGGCGGCGTACGACCAGCTC | |
| | | | CACCTAGAAACCCAGGACCCCCTACCATACGGAGC | |
| | | | CTGCCGGCACTGCCGCCAGCTCCGGAACCCCCTGA | |
| | | | GGAACCACGGCGTGGTGGAGATACAGACGGAGACC | |
| | | | GTGGAGAAGATGGAGGAGACGCCGCTGGGGCCTAC | |
| | | | GAACCCGAAGACCTAGAAGAACTTTTCGCCGCCGC | |
| | | | CGAGCAAGACGATATGTGA | |
| AB064600.1 | BAB79329.1 | ORF2 | ATGTCGTGGAGACCGCCGAGCCAAAATTTACTGCAA | 612 |
| | | | AGAGAAGAGGCCTGGTACTCAGCTTTTCTTAGCTCG | |
| | | | CATTCTACATTTTGCGGTTGTACTGACCCTCTGCTGC | |
| | | | ATATTACTCTCATTGCTGGCCGCCTTACTAACCCCGT | |
| | | | ACCCGTCACCCGCCAACCGGAGACCCCTCCTAACG | |
| | | | GCCTCAGGGGGCTGCCGGCACTGCCAGCACCCCCT | |
| | | | GAACCACCAGCACCGCCACCACGGCCTGGGGATGG | |
| | | | TACCGGAGAAGAAGATGGCGCCCATGGAGAAGGAG | |
| | | | AAGGTGGGCGATACGCAGAAGAAGACCTAGAAGAA | |
| | | | CTGTTCGCCGCCGCGGCAGAAGACGATATGTGA | |
| AB064601.1 | BAB79333.1 | ORF2 | ATGTCGTGGGCTCCGCCGCTATTCAACTCGAAACAG | 613 |
| | | | AGAGAGGACCAGTGGTACCAGTCAATTATTTTCAGC | |
| | | | CATAATACTTTTTGCGGCTGCGGTGACCTTGTTAGG | |
| | | | CATTTTTGCGTCGTTGCTTCTCGCTTTACTGAGCCTC | |
| | | | CTGTAGTGCCGGCCCTACCGGCACCGGTACCGGCA | |
| | | | CCGCCACGGCGTGGTACAGAAGAAGAAGGTGGAGA | |
| | | | CCGTGGAGAAGACGCCGCAGACCGTGGACCCTACG | |
| | | | CAGAAGAAGAGCTAGAAGATTTGTTCGCCGCCGCC | |
| | | | CGAGAAGACGATATGTGA | |
| AB064602.1 | BAB79337.1 | ORF2 | ATGCCGTGGCATCCACCGGGCTACAACGTTCAACA | 614 |
| | | | GAGAGAAGAGCTCTGGGTACAGACAGTTACTACTTC | |
| | | | ACATGCTACTTTTTGCGGCTGTGGTGACCCTAGTAG | |
| | | | CCATCTTCACCGCATTCTTAGCCGCCTTAATAACAG | |
| | | | CAGCCGGCGGCCCCCCGAAACCCCAAACCCCATTC | |
| | | | GTGCCCTACCGGCCCTACCGGCACCCCAAGAACCT | |
| | | | GAACAGCCGCCATCACGGCCTGGTACCGGTACAGA | |
| | | | AGAAGGCCATGGCGCCGAAGGAGGCGACCGAGGT | |
| | | | GGGGCCTACGCAGAAGAAGATTTAGAAGATCTTTTC | |
| | | | GCGGCCGCGGAAGAAGACGATATGTGA | |
| AB064603.1 | BAB79341.1 | ORF2 | ATGTCGTGGCGACCGCCGTTGCATTCTATCCAAGGC | 615 |
| | | | AGAGAAGATCAATGGTATGCAGGCATCTTTCATACG | |
| | | | CATTTTGCTTTTTGCGGTTGTGGTGACCCTGTTGGG | |
| | | | CGTATTAACCGCATTGCTCACCGCTTTCCTAACGCC | |
| | | | GGTCCCCCGAGACCACCTCCAGGGCTAGACCAGCC | |
| | | | CAACCTCGGAGGGCCGGAAGGTCCAGGAGGTGCC | |
| | | | CCTAGAGCCCTGCCAGCCCTGCCGGCCCCGGCAGA | |
| | | | GCCAGAGCCGGCACCACGGCGTGGTGGTGGGGCC | |
| | | | GATGGAGACAGCGCCGCTGGGGCCGCCGCCGCCG | |
| | | | CAGACCATGGAGGGTACGACGAAGGAGACCTAGAA | |
| | | | GATCTTTTCGCCGCCGCCGCCGAGGACGATATGTG | |
| | | | A | |
| AB064604.1 | BAB79345.1 | ORF2 | ATGAGTATTTGGAGGCCTCCACTGCACAATGTCCCG | 616 |
| | | | GGACTCGAACACCTCTGGTACGAGTCAGTGCATCGT | |
| | | | AGCCATGCTGCTGTTTGTGGCTGTGGGGATCCTGTA | |
| | | | CGCCATCTTACTGCTCTTGCTGAAAGATATGGCATT | |
| | | | CCGGGAGGGTCGCGGTCTTCTGGGGCACCGGGAG | |
| | | | TAGGGGGCAACCACAACCCTCCCCAGATCCGTCGA | |
| | | | GCCCGCCACCCGGCGGCTGCTCCGGACCCCCCAG | |
| | | | CAGGTAACCAGCCTCCGGCCCTGCCATGGCATGGG | |
| | | | GATGGTGGAAACGAAAGCGGCGCTGGTGGTGGAGA | |
| | | | AAGCGGTGGACCCGTGGCCGACTTCGCAGACGATG | |
| | | | GCCTAGACGATCTCGTCGCCGCCCTCGACGAAGAA | |
| | | | GAGTAA | |
| AB064606.1 | BAB79353.1 | ORF2 | ATGAGCTTCTGGAGACCTCCGGTGCACAATGCCAC | 617 |
| | | | GGGGATCCAGCGCCTGTGGTACGAGTCCTTTCACC | |
| | | | GTGGCCATGCTGCTTTTTGTGGTTGTGGGGATCCTA | |
| | | | TACTTCACATTACTGCACTTGCTGAGACATATGGCCA | |
| | | | TCCAACAGGCCCGAGACCTTCTGGGCCACCGCGAG | |
| | | | TAGACCCCGATCCCCAGATCCGTAGAGCCAGGCCT | |
| | | | GCCCCGGCCGCTCCGGAGCCCTCACAGGTTGAGCC | |
| | | | GAGACCTGCCCTGCCATGGCATGGGGATGGTGGAA | |
| | | | GCGACGGCGGCGCTGGTGGTTCCGGAAGCGGTGG | |
| | | | ACCCGTGGCAGACTTCGCAGACGATGGCCTCGATC | |
| | | | AGCTCGTCGCCGCCCTAGACGACGAAGAGTAA | |
| DQ003341.1 | AAX94181.1 | ORF2 | ATGGGCAAGGCTCTTAGAGTATTTATTCTTAATATGC | 618 |
| | | | GCTTTTCCAGAATTTACAAACAGAAGAAGAGGCCAC | |
| | | | TGCCACTGCTTCTGGTGCGAGTTGAACCGAAAGCAT | |
| | | | TCGCTAGTGATATGAGTTGGCGCCCTCCCGTTCACA | |
| | | | ATGCGGCAGGAATTGAGCGACAGCTCCTTGAGGGC | |
| | | | TGCTTTCGATTTCACGCTGCCTGTTGCGGTTGTGGC | |
| | | | AGTTTTATTACTCATCTTACTATACTGGCTGCTCGCT | |
| | | | ATGGTTTTACTGGGGGGCCGGCGCCGCCAGGTGGT | |
| | | | CCTGGGGCGCTGCCATCGCTGAGACGGGCTCAGCC | |
| | | | CGCGCCGGCGGCCCCCGAGAACCAGCCTGAACCA | |
| | | | GAGCTATGGCGTGGTCGTGGTGGTGGAGGCGACG | |
| | | | GAAACGCTGGTGGCCGCGCAGAAGGAGGCGATGG | |
| | | | AGGAGATTTCGCACCCGAAGAGCTAGACGAGCTGTT | |
| | | | CCGCGCCGTCGCCGCCGACGAAGAGTAA | |
| DQ003342.1 | AAX94184.1 | ORF2 | ATGGGCAAGGCTCTTAGAGTATTTATTCTTAATATGC | 619 |
| | | | GCTTTTCCAGAATTTACAAACAGAAGAAGAGGCCAC | |
| | | | TGCCACTGCTTCTGGTGCGAGTTGAACCGAAAGCAT | |
| | | | TCGCTAGTGATATGAGTTGGCGCCCTCCCGTTCACA | |
| | | | ATGCGGCAGGAATTGAGCGACAGCTCCTTGAGGGC | |
| | | | TGCTTTCGATTTCACGCTGCCTGTTGCGGTTGTGGC | |
| | | | AGTTTTATTACTCATCTTACTATACTGGCTGCTCGCT | |
| | | | ATGGTTTTACTGGGGGGCCGGCGCCGCCAGGTGGT | |
| | | | CCTGGGGCGCTGCCATCGCTGAGACGGGCTCAGCC | |
| | | | CGCGCCGGCGGCCCCCGAGAACCAGCCTGAACCA | |
| | | | GAGCTATGGCGTGGTCGTGGTGGTGGAGGCGACG | |
| | | | GAAACGCTGGTGGCCGCGCAGAAGGAGGCGATGG | |
| | | | AGGAGATTTCGCACCCGAAGAGCTAGACGAGCTGTT | |
| | | | CCGCGCCGTCGCCGCCGACGAAGAGTAA | |
| DQ003343.1 | AAX94187.1 | ORF2 | ATGGGCAAGGCTCTTAGAGTATTCATTCTTAATATGC | 620 |
| | | | GCTTTTCCAGAATTTACAAACAGAAGAAGAGGCCAC | |
| | | | TGCCACTGCTTCTGGTGCGAGTTGAACCGAAAGCAC | |
| | | | TCGCTAGTGATATGAGTTGGCGCCCTCCCGTTCACA | |
| | | | ATGCGGCAGGAATTGAGCGACAGCTCCTTGAGGGC | |
| | | | TGCTTTCGATTTCACGCTGCCTGTTGCGGTTGTGGC | |
| | | | AGTTTTATTACTCATCTTACTATACTGGCTGCTCGCT | |
| | | | ATGGTTATACTGGGGGGCCGGCGCCGCCAGGTGGT | |
| | | | CCTGGGGCGCTGCCATCGCTGAGACGGGCTCTGCC | |
| | | | CGCGCCGGCGGCCCCCGAGAACCAGCCTGAACCA | |
| | | | GAGCTATGGCGTGGTCGTGGTGGTGGAGGCGACG | |
| | | | GAAACGCTGGTGGCCGCGCAGAAGGAGGCGATGG | |
| | | | AGGAGATTTCGCACCCGAAGAGCTAGACGAGCTGTT | |
| | | | CCGCGCCGTCGCCGCCGACGAAGAGTAA | |
| DQ003344.1 | AAX94190.1 | ORF2 | ATGGGCAAGGCTCTTAGAGTATTCATTCTTAATATGC | 621 |
| | | | GCTTTTCCAGAATTTACAAACAGAAGAAGAGGCCAC | |
| | | | TGCCACTGCTTCTGGTGCGAGTTGAACCGAAAGCAC | |
| | | | TCGCTAGTGATATGAGTTGGCGCCCTCCCGTTCACA | |
| | | | ATGCGGCAGGAATTGAGCGACAGCTCCTTGAGGGC | |
| | | | TGCTTTCGATTTCACGCTGCCTGTTGCGGTTGTGGC | |
| | | | AGTTTTATTACTCATCTTACTATACTGGCTGCTCGCT | |
| | | | ATGGTTATACTGGGGGGCCGGCGCCGCCAGGTGGT | |
| | | | CCTGGGGCGCTGCCATCGCTGAGACGGGCTCTGCC | |
| | | | CGCGCCGGCGGCCCCCGAGAACCAGCCTGAACCA | |
| | | | GAGCTATGGCGTGGTCGTGGTGGTGGAGGCGACG | |
| | | | GAAACGCTGGTGGCCGCGCAGAAGGAGGCGATGG | |
| | | | AGGAGATTTCGCACCCGAAGAGCTAGACGAGCTGTT | |
| | | | CCGCGCCGTCGCCGCCGACGAAGAGTAA | |
| DQ186994.1 | ABD34285.1 | ORF2 | ATGGGCAAGGCTCTTAGAGTATTCATTCTTAATATGC | 622 |
| | | | GCTTTTCCAGAATTTACAAACAGAAGAAGAGGCCAC | |
| | | | TGCCACTGCTTCTGGTGCGAGTTGAACCGAAAGCAC | |
| | | | TCGCTAGTGATATGAGTTGGCGCCCTCCCGTTCACA | |
| | | | ATGCGGCAGGAATTGAGCGACAGCTCCTTGAGGGC | |
| | | | TGCTTTCGATTTCACGCTGCCTGTTGCGGTTGTGGC | |
| | | | AGTTTTATTACTCATCTTACTATACTGGCTACTCGCT | |
| | | | ATGGTTTTACTGGGGGGCCGGCGCCGCCAGGTGGT | |
| | | | CCTGGGGCGCTGCCATCGCTGAGACGGGCTCTGCC | |
| | | | CGCGCCGGCGGCCCCCGAGAACCAGCCTGAACCA | |
| | | | GAGCTATGGCGTGGTCGTGGTGGTGGAGGCGACG | |
| | | | GAAACGCTGGTGGCCGCGCAGAAGGAGGCGATGG | |
| | | | AGGAGATTTCGCACCCGAAGAGCTAGACGAGCTGTT | |
| | | | CCGCGCCGTCGCCGCCGACGAAGAGTAA | |
| DQ186995.1 | ABD34287.1 | ORF2 | ATGGGCAAGGCTCTTAGAGTATTCATTCTTAATATGC | 623 |
| | | | GCTTTTCCAGAATTTACAAACAGAAGAAGAGGCCAC | |
| | | | TGCCACTGCTTCTGGTGCGAGTTGAACCGAAAGCAC | |
| | | | TCGCTAGTGATATGAGTTGGCGCCCTCCCGTTCACA | |
| | | | ATGCGGCAGGAATTGAGCGACAGCTCCTTGAGGGC | |
| | | | TGCTTTCGATTTCACGCTGCCTGTTGCGGTTGTGGC | |
| | | | AGTTTTATTACTCATCTTACTATACTGGCTACTCGCT | |
| | | | ATGGTTTTACTGGGGGGCCGGCGCCGCCAGGTGGT | |
| | | | CCTGGGGCGCTGCCATCGCTGAGACGGGCTCTGCC | |
| | | | CGCGCCGGCGGCCCCCGAGAACCAGCCTGAACCA | |
| | | | GAGCTATGGCGTGGTCGTGGTGGTGGAGGCGACG | |
| | | | GAAACGCTGGTGGCCGCGCAGAAGGAGGCGATGG | |
| | | | AGGAGATTTCGCACCCGAAGAGCTAGACGAGCTGTT | |
| | | | CCGCGCCGTCGCCGCCGACGAAGAGTAA | |
| DQ186996.1 | ABD34289.1 | ORF2 | ATGGGCAAGGCTCTTAGGGTCTTCATTCTTAATATGT | 624 |
| | | | TCCTTGGCAGGGTTTACCGCCACAAGAAAAGGAAAG | |
| | | | TGCTACTGTCTACACTGCGAGCTCCACAGGCGTCTC | |
| | | | GCAGGGCTATGAGTCGGCGACCCCCGGTACACGAT | |
| | | | GCACCCGGCATCGAGCGCAATTGGTACGAGGCCTG | |
| | | | TTTCAGAGCCCACGCTGGAGCTTGTGGCTGTGGCA | |
| | | | ATTTTATTATGCACCTTAATCTTCTGGCTGGGCGTTA | |
| | | | TGGTTTTACTCCGGGGTCAGCGCCGCCAGGTGGTC | |
| | | | CTCCTCCGGGCACCCCGCAGATAAGAAGAGCCAGA | |
| | | | CCTAGTCCCGCCGCACCCCAAGAGCCCGCTGCTCT | |
| | | | ACCATGGCATGGGGATGGTGGAGATGGCGGCGCC | |
| | | | GCTGGCCCGCCAGACGCTGGAGGAGACGCCGTCG | |
| | | | CCGGCGCCCCGTACGGAGAACAAGAGCTCGCCGAC | |
| | | | CTGCTCGACGCTATAGAAGACGACGAACAGTAA | |
| DQ186997.1 | ABD34291.1 | ORF2 | ATGGGCAAGGCTCTTAGGGTCTTCATTCTTAATATGT | 625 |
| | | | TCCTTGGCAGGGTTTACCGCCACAAGAAAAGGAAAG | |
| | | | TGCTACTGTCCACACTGCGAGCTCCACAGGCGTCTC | |
| | | | GCAGGGCTATGAGTTGGCGACCCCCGGTACACGAT | |
| | | | GCACCCGGCATCGAGCGCAATTGGTACGAGGCCTG | |
| | | | TTTCAGAGCCCACGCTGGAGCTTGTGGCTGTGGCA | |
| | | | ATTTTATTATGCACCTTAATCTTCTGGCTGGGCGTTA | |
| | | | TGGTTTTACTCCGGGGTCAGCGCCGCCAGGTGGTC | |
| | | | CTCCTCCGGGCACCCCGCAGATAAGAAGAGCCAGA | |
| | | | CCTAGTCCCGCCGCACCCCAAGAGCCCGCTGCTCT | |
| | | | ACCATGGCATGGGGATGGTGGAGATGGCGGCGCC | |
| | | | GCTGGCCCGCCAGACGCTGGAGGAGACGCCGTCG | |
| | | | CCGGCGCCCCGTACGGAGAACAAGAGCTCGCCGAC | |
| | | | CTGCTCGACGCTATAGAAGACGACGAACAGTAA | |
| DQ186998.1 | ABD34293.1 | ORF2 | ATGGGCAAGGCTCTTAGGGTCTTCATTCTTAATATGT | 626 |
| | | | TCCTTGGCAGGGTTTACCGCCACAAGAAAAGGAAAG | |
| | | | TGCTACTGTCCACACTGCGAGCTCCACAGGCGTCTC | |
| | | | GCAGGGCTATGAGTTGGCGACCCCCGGTACACGAT | |
| | | | GCACCCGGCATCGAGCGCAATTGGTACGAGGCCTG | |
| | | | TTTCAGAGCCCACGCTGGGGCTTGTGGCTGTGGCA | |
| | | | ATTTTATTATGCACCTTAATCTTCTGGCTGGGCGTTA | |
| | | | TGGTTTTACTCCGGGGTCAGCGCCGCCAGGTGGTC | |
| | | | CTCCTCCGGGCACCCCGCAGATAAGAAGAGCCAGA | |
| | | | CCTAGTCCCGCCGCACCCCAAGAGCCCGCTGCTCT | |
| | | | ACCATGGCATGGGGATGGTGGAGATGGCGGCGCC | |
| | | | GCTGGCCCGCCAGACGCTGGAGGAGACGCCGTCG | |
| | | | CCGGCGCCCCGTACGGAGAACAAGAGCTCGCCGAC | |
| | | | CTGCTCGACGCTATAGAAGACGACGAACAGTAA | |
| DQ186999.1 | ABD34295.1 | ORF2 | ATGCACTTTTCTCGAATAAGCAGAAAGAAAAGGAAA | 627 |
| | | | GTGCTACTGCTTTGCGTGCCAGCAGCTAAGAAAAAA | |
| | | | CCAACTGCTATGAGCTTCTGGAGACCTCCGGTGCAC | |
| | | | AATGTCACGGGGATCCAGCGCCTGTGGTACGAGTC | |
| | | | CTTTCACCGTGGCCATGCTGCTTTTTGTGGTTGTGG | |
| | | | GGATCCTATACTTCACATTACTTCACTTGCTGAGACA | |
| | | | TATGGCCATCCAACAGGCCCGAGACCTTCTGGGTCA | |
| | | | TCGGGAATAGACCCCACTCCGCCCATCCGTAGAGC | |
| | | | CAGGCCTGCCCCGGCCGCTCCGGAACCCTCACAGG | |
| | | | TTGACTCCAGACCGGCCCTGCCATGGCATGGAGAT | |
| | | | GGTGGAAGCGACGGAGGCGCTGGTGGTTCCGCAA | |
| | | | GCGGTGGACCCGTGGCAGACTTCGCAGACGATGGC | |
| | | | CTCGACCAGCTCGTCGCCGACCTAGACGACGAAGA | |
| | | | GTAA | |
| DQ187000.1 | ABD34297.1 | ORF2 | ATGCACTTTTCTCGAATAAGCAGAAAGAAAAGGAAA | 628 |
| | | | GTGCTACTGCTTTGCGTGCCAGCAGCTAAGAAAAAA | |
| | | | CCAACTGCTATGAGCTTCTGGAGACCTCCGGTGCAC | |
| | | | AATGTCACGGGGATCCAGCGCCTGTGGTACGAGTC | |
| | | | CTTTCACCGTGGCCATGCTGCTTTTTGTGGTTGTGG | |
| | | | GGATCCTATACTTCACATTACTTCACTTGCTGAGACA | |
| | | | TATGGCCATCCAACAGGCCCGAGACCTTCTGGGTCA | |
| | | | TCGGGAATAGACCCCACTCCGCCCATCCGTAGAGC | |
| | | | CAGGCCTGCCCCGGCCGCTCCGGAACCCTCACAGG | |
| | | | TTGACTCCAGACCGGCCCTGCCATGGCATGGAGAT | |
| | | | GGTGGAAGCGACGGAGGCGCTGGTGGTTCCGCAA | |
| | | | GCGGTGGACCCGTGGCAGACTTCGCAGACGATGGC | |
| | | | CTCGACCAGCTCGTCGCCGACCTAGACGACGAAGA | |
| | | | GTAA | |
| DQ187001.1 | ABD34299.1 | ORF2 | ATGCACTTTTCTCGAATAAGCAGAAAGAAAAGGAAA | 629 |
| | | | GTGCTACTGCTTTGCGTGCCAGCAGCTAAGAAAAAA | |
| | | | CCAACTGCTATGAGCTTCTGGAGACCTCCGGTGCAC | |
| | | | AATGTCACGGGGATCCAGCGCCTGTGGTACGAGTC | |
| | | | CTTTCACCGTGGCCATGCTGCTTTTTGTGGTTGTGG | |
| | | | GGATCCTATACTTCACATTACTTCACTTGCTGAGACA | |
| | | | TATGGCCATCCAACAGGCCCGAGACCTTCTGGGTCA | |
| | | | TCGGGAATAGACCCCACTCCGCCCATCCGTAGAGC | |
| | | | CAGGCCTGCCCCGGCCGCTCTGGAACCCTCACAGG | |
| | | | TTGACTCCAGACCGGCCCTGCCATGGCACGGAGAT | |
| | | | GGTGGAAGCGACGGAGGCGCTGGTGGTTCCGCAA | |
| | | | GCGGTGGACCCGTGGCAGACTTCGCAGACGATGGC | |
| | | | CTCGACCAGCTCGTCGCCGACCTAAACGACGAAGA | |
| | | | GTAA | |
| DQ187002.1 | ABD34301.1 | ORF2 | ATGCACTTTTCTCGAATAAGCAGAAAGAAAAGGAAA | 630 |
| | | | GTGCTACTGCTTTGCGTGCCAGCAGCTAAGAAAAAA | |
| | | | CCAACTGCTATGAGCTTCTGGAGACCTCCGGTGCAC | |
| | | | AATGTCACGGGGATCCAGCGCCTGTGGTACGAGTC | |
| | | | CTTTCACCGTGGCCATGCTGCTTTTTGTGGTTGTGG | |
| | | | GGATCCTATACTTCACATTACTTCACTTGCTGAGACA | |
| | | | TATGGCCATCCAACAGGCCCGAGACCTTCTGGGTCA | |
| | | | TCGGGAATAGACCCCACTCCGCCCATCCGTAGAGC | |
| | | | CAGGCCTGCCCCGGCCGCTCCGGAACCCTCACAGG | |
| | | | TTGACTCCAGACCGGCCCTGCCATGGCATGGAGAT | |
| | | | GGTGGAAGCGACGGAGGCGCTGGTGGTTCCGCAA | |
| | | | GCGGTGGACCCGTGGCAGACTTCGCAGACGATGGC | |
| | | | CTCGACCAGCTCGTCGCCGACCTAAACGACGAAGA | |
| | | | GTAA | |
| DQ187003.1 | ABD34303.1 | ORF2 | ATGCACTTTTCTCGAATAAGCAGAAAGAAAAGGAAA | 631 |
| | | | GTGCTACTGCTTTGCGTGCCAGCAGCTAAGAAAAAA | |
| | | | CCAACTGCTATGAGCTTCTGGAGACCTCCGGTGCAC | |
| | | | AATGTCACGGGGATCCAGCGCCTGTGGTACGAGTC | |
| | | | CTTTCACCGTGGCCATGCTGCTTTTTGTGGTTGTGG | |
| | | | GGATCCTATACTTCACATTACTTCACTTGCTGAGACA | |
| | | | TATGGCCATCCAACAGGCCCGAGACCTTCTGGGTCA | |
| | | | TCGGGAATAGACCCCACTCCGCCCATCCGTAGAGC | |
| | | | CAGGCCTGCCCCGGCCGCTCCGGAACCCTCACAGG | |
| | | | TTGACTCCAGACCGGCCCTGCCATGGCATGGAGAT | |
| | | | GGTGGAAGCGACGGAGGCGCTGGTGGTTCCGCAA | |
| | | | GCGGTGGACCCGTGGCAGACTTCGCAGACGATGGC | |
| | | | CTCGACCAGCTCGTCGCCGACCTAGACGACGAAGA | |
| | | | GTAA | |
| DQ187004.1 | ABD34304.1 | ORF2 | ATGTTTTTCGGTAGACATTGGCGAAAGAAAAGGGCA | 632 |
| | | | CTGTTACTGTCTAGCTTGCGAACTTCAAAGAAGAAA | |
| | | | CCACCTGCAATGAGCCAGTGGTGCCCGCCTGTGCA | |
| | | | CAGCGTTCAGGGTCGCAACCACCAGTGGTATGAAG | |
| | | | CCTGCTACCGTGGCCATGCTGCTTATTGTGGCTGTG | |
| | | | GCGATTTTATTAGTCACCTTGTTGCTCTGGGTAATCA | |
| | | | GTTTGGCTTCAGGCCGGGTCCCCGAGCTCCTGGCG | |
| | | | CACCGGGGCTAGGGGGACCCCCCGTTCTGCCCCGT | |
| | | | AGAGCCCTGCCGGCACCCCCGGCTGAGGCTCCGG | |
| | | | AGCACCAGCAGGGCAACAACAACAACAACCAGCAG | |
| | | | CTGCAGAGATGGCCTGGGGATGGTGGAAACGCAGA | |
| | | | CGGCGCCGATGGTGGAGAGGCCTCTGGAGGAGAC | |
| | | | GCCGCTTTGCCAGAAGACGACCTAGACGGCCTGCT | |
| | | | CGCCGCCCTAGACGACGAAGAGTAA | |
| DQ187005.1 | ABD34306.1 | ORF2 | ATGTTTTTCGGTAGGCATTGGCGAAAGAAAAGGGCA | 633 |
| | | | CTGTTACTGTCTAGCTTGCGAACTTCAAAGAAGAAA | |
| | | | CCACCTGCAATGAGCCAGTGGTGCCCGCCTGTGCA | |
| | | | CAGCGTTCAGGGTCGCAACCACCAGTGGTATGAAG | |
| | | | CCTGCTACCGTGGCCATGCTGCTTATTGTGGCTGTG | |
| | | | GCGATTTTATTAGTCACCTTGTTGCTCTGGGTAATCA | |
| | | | GTTTGGCTTCGGGCCGGGTCCCCGAGCTCCTGGCG | |
| | | | CACCGGGGCTAGGGGGACCCCCCGTTCTGCCCCGT | |
| | | | AGAGCCCTGCCGGCACCCCCGGCTGAGGCTCCGG | |
| | | | AGCACCAGCAGGGCAACAACAACAACAACCAGCAG | |
| | | | CTGCAGAGACGGCCTGGGGATGGTGGAAACGCAGA | |
| | | | CGGCGCCGATGGTGGAGAGGCCTCTGGAGGAGAC | |
| | | | GCCGCTTTGCCAGAAGACGACCTAGACGGCCTGCT | |
| | | | CGCCGCCCTAGACGACGAAGAGTAA | |
| DQ187007.1 | ABD34309.1 | ORF2 | ATGTTTTTCGGTAGGCATTGGCGAAAGAAAAGGGCA | 634 |
| | | | CTGTTACTGTCTAGCTTGCGAACTTCAAAGAAGAAA | |
| | | | CCACCTGCAATGAGCCAGTGGTGCCCGCCTGTGCA | |
| | | | CAGCGTTCAGGGTCGCAACCACCAGTGGTATGAAG | |
| | | | CCTGCTACCGTGGCCATGCTGCTTATTGTGGCTGTG | |
| | | | GCGATTTTATTAGTCACCTTGTTGCTCTGGGTAATCA | |
| | | | GTTTGGCTTCAGGCCGGGTCCCCGAGCTCCTGGCG | |
| | | | CACCGGGGCTAGGGGGACCCCCCGTTCTGCCCCGT | |
| | | | AGAGCCCTGCCGGCACCCCCGGCTGAGGCTCCGG | |
| | | | AGCACCAGCAGGGCAACAACAACAACAACCAGCAG | |
| | | | CTGCAGAGATGGCCTGGGGATGGTGGAAACGCAGA | |
| | | | CGGCGCCGATGGTGGAGAGGCCTCTGGAGGAGAC | |
| | | | GCCGCTTTGCCAGAAGACGACCTAGACGGCCTGCT | |
| | | | CGCCGCCCTAGACGACGAAGAGTAA | |
| EF538879.1 | ABU55886.1 | ORF2 | ATGCACTTTTCTCGAATAAGCAGAAAGAAAAGGAAA | 635 |
| | | | GTGCTACTGCTTTGCGTGCCAGCAGCTAAGAAACAA | |
| | | | CCAACTGCTATGAGCTTCTGGAGACCTCCGATACAC | |
| | | | AATGTCACGGGGATCCAGCGCCTGTGGTACGAGTC | |
| | | | CTTTCACCGTGGCCATGCTGCTTTTTGTGGTTGTGG | |
| | | | GGATCCTATACTTCACATTACTGCACTTGCTGAGACA | |
| | | | TATGGCCATCCAACAGGCCCGAGACCTTCTGGGTCA | |
| | | | TCGGGAATAGACCCCACTCCCCCAATCCGTAGAGC | |
| | | | CAGGCCCGCCCCGGCCGCTCCGGAGCCCTCACAG | |
| | | | GCTGAGTCCAGACCGGCCCTGCCATGGCATGGAGA | |
| | | | TGGTGGAAGCGACGGAGGCGCTGGTGGTTCCGCAA | |
| | | | GCGGTGGACCCGTGGCAGACTTCGCAGACGATGGC | |
| | | | CTCGACCAGCTCGTCGCCGCCCTAGACGACGAAGA | |
| | | | GTAA | |
| FJ426280.1 | ACK44072.1 | ORF2 | ATGTTTCTCGGCAGGGTGTGGAGGAAACAGAAAAG | 636 |
| | | | GAAAGTGCTTCTGCTGGCTGTGCGAGCTACACAGAA | |
| | | | AACATCTTCCATGAGTATCTGGCGTCCCCCTCTCGG | |
| | | | GAATGTCTCCTACAGGGAGAGAAATTGGCTTCAGGC | |
| | | | CGTCGAAGGATCCCACAGTTCCTTTTGTGGCTGTGG | |
| | | | TGATTTTATTCTTCATCTTACTAATTTGGCTGCACGC | |
| | | | TTTGCTCTTCAGGGGCCCCCGCCGGAGGGTGGTCC | |
| | | | TCCTCGGCCGAGGCCGCCGCTCCTGAGAGCGCTGC | |
| | | | CGGCCCCCGAGGTCCGCAGGGAAACGCGCACAGA | |
| | | | GAACCCGGGCGCCTCCGGTGAGCCATGGCCTGGC | |
| | | | GATGGTGGTGGCAGAGACGATGGCGCCGCCGCCC | |
| | | | GTGGCCCCGCAGACGGTGGAGACGCCTACGACGC | |
| | | | CGGAGACCTCGACGACCTGTTCGCCGCCGTCGAAG | |
| | | | ACGAGCAACAGTAA | |
| FJ392105.1 | ACR20258.1 | ORF2 | CTGCCACTGCTACCTGTGCCAGCTACACCGCAAGAA | 637 |
| | | | CGGCCTAGTCGTGCGCCCCTGATGGCCTGCGGACC | |
| | | | CAGAGGATGGATGCCCCCCAACTTCGGGGGACACG | |
| | | | ACAGAGAAAATGCTTGGTGCAAATCTGTTAAATTGTC | |
| | | | TCATGATGCTTTCTGTGGCTGCGACGATCCTCTTAC | |
| | | | CCATCTTGCTGCTCTGCTACCAAGCAGACAAGCTTC | |
| | | | TCGTCAGAATACTCCTTCTGCTCCACCTCCGCGCCC | |
| | | | CCCGCCGCCGACCCCGAGGCAGGGCCAGGGCTCT | |
| | | | GGGCCGCCTCAGGGGCGAATCAGACCGTCCTGGTC | |
| | | | CCTCCCGGTGACCCCACCCGCTGACGAGCCATGGC | |
| | | | AGCCTGGTGGTGGGGCAGGCGGAGACGCTGGCGC | |
| | | | AGGTGGAGGCGCCGCCGCCTCCCTCGCCGCCGCC | |
| | | | GCTGGCGACGGAGGAGACGGTGGCCCAGAAGACG | |
| | | | CAGGCGGAGATGGCCGCGCAGACGCAGACGTCGC | |
| | | | AGACCTGCTCGCCGCCCTAGAAGGAGACGCAGACG | |
| | | | CCGAAGGGTAA | |
| FJ392107.1 | ACR20261.1 | ORF2 | GATCCTCTTACCCATCTTGCTGCTCTGCTACCAGGC | 638 |
| | | | AGACAAGCTTCTCGTCAGAATACTCCTTCTGCTCCA | |
| | | | CCTCCGCGCCCCCCGCCGCCGACCCCGAGGCAGG | |
| | | | GCCAGGGCTCTGGGCCGCCTCAGGGGCGAATCAGA | |
| | | | CCGTCCTGGTCCCTCCCGGTGACCCCACCCGCTGA | |
| | | | CGAGCCATGGCAGCCTGGTGGTGGGGCAGGCGGA | |
| | | | GACGCTGGCGCAGGTGGAGGCGCCGCCGCCTCCC | |
| | | | TCGCCGCCGCCGCTGGCGACGGAGGAGACGGTGG | |
| | | | CCCAGAAGACGCAGGCGGAGATGGCCGCGCAGAC | |
| | | | GCAGACGTCGCAGACCTGCTCGCCGCCCTAGAAGG | |
| | | | AGACGCAGACGCCGAAGGGTAA | |
| FJ392108.1 | ACR20263.1 | ORF2 | TCTCATGATGCTTTCTGTGGCTGCGACGATCCTCTT | 639 |
| | | | ACCCATCTTGCTGCTCTGCTACCAGGCAGACAAGCT | |
| | | | TCTCGTCAGAATACTCCTTCTGCTCCACCTCCGCGC | |
| | | | CCCCCGCCGCCGACCCCGAGGCAGGGCCAGGGCT | |
| | | | CTGGGCCGCCTCAGGGGCGAATCAGACCGTCCTGG | |
| | | | TCCCTCCCGGTGACCCCACCCGCTGACGAGCCATG | |
| | | | GCAGCCTGGTGGTGGGGCAGGCGGAGACGCTGGC | |
| | | | GCAGGTGGAGGCGCCGCCGCCTCCCTCGCCGCCG | |
| | | | CCGCTGGCGACGGAGGAGACGGTGGCCCAGAAGA | |
| | | | CGCAGGCGGAGATGGCCGCGCAGACGCAGACGTC | |
| | | | GCAGACCTGCTCGCCGCCCTAGAAGGAGACGCAGA | |
| | | | CGCCGAAGGGTAA | |
| FJ392111.1 | ACR20268.1 | ORF2 | CAAGAACGGCCTAGTCGTGCGCCCCTGATGGCCTG | 640 |
| | | | CGGACCCAGAGGATGGATGCCCCCCAACTTCGGGG | |
| | | | GACACGACAGAGAAAATGCTTGGTGCAAATCTGTTA | |
| | | | AATTGTCTCATGATGCTTTCTGTGGCTGCGACGATC | |
| | | | CTCTTACCCATCTTGCTGCTCTGCTACCAGGCAGAC | |
| | | | AAGCTTCTCGCCAGAATACTCCTTCTGCTCCACCTC | |
| | | | CGCGCCCCCCGCCGCCGACCCCGAGGCAGGGCCA | |
| | | | GGGCTCTGGGCCGCCTCAGGGGCGAATCAGACCGT | |
| | | | CCTGGTCCCTCCCGGTGACCCCACCCGCTGACGAG | |
| | | | CCATGGCAGCCTGGTGGTGGGGCAGGCGGAGACG | |
| | | | CTGGCGCAGGTGGAGGCGCCGCCGCCTCCCTCGC | |
| | | | CGCCGCCGCTGGCGACGGAGGAGACGGTGGCCCA | |
| | | | GAAGACGCAGGCGGAGATGGCCGCGCAGACGCAG | |
| | | | ACGTCGCAGACCTGCTCGCCGCCCTAGAAGGAGAC | |
| | | | GCAGACGCCGAAGGGTAA | |
| FJ392112.1 | ACR20270.1 | ORF2 | CTGCTACCTGTGCCAGCTACACCGCAAGAACGGCC | 641 |
| | | | TAGTCGTGCGCCCCTGATGGCCTGCGGACCCAGAG | |
| | | | GATGGATGCCCCCCAACTTCGGGGGACACGACAGA | |
| | | | GAAAATGCTTGGTGCAAATCTGTTAAATTGTCTCATG | |
| | | | ATGCTTTCTGTGGCTGCGACGATCCTCTTACCCATC | |
| | | | TTGCTGCTCTGCTACCAGGCAGACAAGCTTCTCGTC | |
| | | | AGAATACTCCTTCTGCTCCACCTCCGCGCCCCCCGC | |
| | | | CGCCGACCCCGAGGCAGGGCCAGGGCTCTGGGCC | |
| | | | GCCTCAGGGGCGAATCAGACCGTCCTGGTCCCTCC | |
| | | | CGGTGACCCCACCCGCTGACGAGCCATGGCAGCCT | |
| | | | GGTGGTGGGGCAGGCGGAGACGCTGGCGCAGGTG | |
| | | | GAGGCGCCGCCGCCTCCCTCGCCGCCGCCGCTGG | |
| | | | CGACGGAGGAGACGGTGGCCCAGAAGACGCAGGC | |
| | | | GGAGATGGCCGCGCAGACGCAGACGTCGCAGACCT | |
| | | | GCTCGCCGCCCTAGAAGGAGACGCAGACGCCGAAG | |
| | | | GGTAA | |
| FJ392113.1 | ACR20271.1 | ORF2 | ATGTTCCTCGGCAGGCCGTGGAGAAAGAGGAGGGC | 642 |
| | | | GGCCGGGAAGAAAGGGCCACTGCCACTGCAAGCTG | |
| | | | TGCGAGCTGCATCGCAGGAACGGTCTGACAGTGCA | |
| | | | CCGCTGATGGCCTGCGGACCCCGGGGATGGATGCC | |
| | | | CCCGAACTTCGGGGGACACGAGAGAGAAAATGCCT | |
| | | | GGAGCCAGTCTGTTGTACTGTCTCATGATGCTTTCT | |
| | | | GTGGCTGCGACGATCCTGCTACCCATCTTACTGCTC | |
| | | | TGCTATCAGGTAGACAAGCTTCTCGTCAGAGTACTC | |
| | | | CTTCTGCTCCACCTCCGCGCCCCCCGCCGCCGTCC | |
| | | | CCGAGGCAGGGCCAGGGGTCTCGGTCACCTCCGG | |
| | | | GGCGAATCAGACCATCCTGGTCCCTCCCGGTAGCC | |
| | | | CCGCCGAGTGAAGGGCCATGGCTGCCTGGTGGTGG | |
| | | | GGCAGGAGGCGGCGATGGCGCCGGTGGAGACGGC | |
| | | | GCCGTCTCCCTCGCCGCCGCCGCTGGTGACGGAG | |
| | | | GAGACGGTGGCCCAGGAGGCGTAGGCGGAGATGG | |
| | | | CCGCGGAGACGCAGACGTCGCAGACCTGCTCGCCG | |
| | | | CCTTAGAAGGAGACGTCGACGCAGAAGGGTAA | |
| FJ392114.1 | ACR20273.1 | ORF2 | ATGTTCCTCGGCAGGCCGTGGAGAAAGAGGAGGGC | 643 |
| | | | GGCCGGGAAGAAAGGGCCACTGCCACTGCAAGCTG | |
| | | | TGCGAGCTGCATCGCAGGAACGGTCTCACAGTGCA | |
| | | | CCGCTGATAGCCTGCGGACCCCGGGGATGGATGCC | |
| | | | CCCGAACTTCGGGGGACACGAGAGGGAAAATGCCT | |
| | | | GGAGCCAGTCTGTTGTACTGTCTCATGATGCTTTCT | |
| | | | GTGGTTGCGACGATCCTGCTACCCATCTTACTACTC | |
| | | | TGCTATCACGCAGACAAGCTTCTCGTCAGAGTACTC | |
| | | | CTTCTGCTCCACCTCCGCGCCCCCCGCCGCCGTCC | |
| | | | CCGAGGCAGGGCCAGGGGTCTCGGTCGCCTCCGG | |
| | | | GACGAATCAGACCATCCTGGTCCCTCCCGGTAGCC | |
| | | | CCGCCGAGTGAAGGGCCATGGCTGCCTGGTGGTGG | |
| | | | GGCAGGAGGCGGCGATGGCGCCGGTGGAGACGGC | |
| | | | GCCGTCTCCCTCGCCGCCGCCGCTGGCGACGGAG | |
| | | | GAGACGGTGGCCCAGGAGGCGTAGGCGGAGATGG | |
| | | | CCGCGGAGACGCAGACGTCGCGGACCTGCTCGCC | |
| | | | GCCTTAGAAGGAGACGTCGACGCAGAAGGGTAA | |
| FJ392115.1 | ACR20275.1 | ORF2 | ATGTTCCTCGGCAGGCCGTGGAGAAAGAGGAGAGC | 644 |
| | | | GGCAGGGAAGAAAGGGCCACTGCCACTGCAAGCTG | |
| | | | TGCGGGCTGCATCGCAGGAACGGTCTCACAGTGCA | |
| | | | CCGCTGATGGCCTGCGGACCCCGGGGATGGATGCC | |
| | | | CCCGAACTTCGGGGGACACGAGAGAGAAAATGCCT | |
| | | | GGAGCCAGTCTGTTGTACTGTCTCATGATGCTTTCT | |
| | | | GTGGTTGCGACGATCCTGCTACCCATCTTACTACTC | |
| | | | TGCTATCACGCAGACAAGCTTCTCGTCAGAGTACTC | |
| | | | CTTCTGCTCCACCTCCGCGCCCCCCGCCGCCGTCC | |
| | | | CCGAGGCAGGGCCAGGGGTCTCGGTCGCCTCCGG | |
| | | | GGCGAATCAGACCATCCTGGTCCCTCCCGGTAGCC | |
| | | | CCGCCGAGTGAAGGGCCATGGCTGCYTGGTGGTGG | |
| | | | GGCAGGAGGCGGCGATGGCGCCGGTGGAGACGGC | |
| | | | GCCGTYTCCCTCGCCGCCGCCGCTGGCGACGGAG | |
| | | | GAGACGGTGGCCCAGGAGGCGTAGGCGGAGATGG | |
| | | | CCGCGGAGACGCAGACGTCGCAGACCTGCTCGCCG | |
| | | | CCTTAGAAGGAGACGTCGACGCAGAAGGGTAA | |
| GU797360.1 | AD051764.1 | ORF2 | ATGGCTGAGTTTATGCTGCCCGTCCGCAGAGAGGA | 645 |
| | | | GCCACGGCGGGGGATCCGAACGTCCCGAGGGCGG | |
| | | | GTGCCGGAGGTGAGTTTACACACCGCAGTCAAGGG | |
| | | | GCAATTCGGGCTCGGGACTGGCCGGGCTATGGGCA | |
| | | | AGGCTCTTAAAAAAGCCATGTTTCTCGGTAAATTACA | |
| | | | CAGAAAGAAGAGGGCACTGTCACTGCACGGCCTGC | |
| | | | CAGCTACAAAGAAAAAACCACCTCCTGATATGAACT | |
| | | | ACTGGAGGCCGCCTGTGCACAATGTCCCGGGGCTC | |
| | | | GAACGCCTCTGGTACGAGTCCGTGCATCGTAGCCAT | |
| | | | GCTGCTGTTTGTGGTTGTGGGGATTTTGTACGCCAT | |
| | | | ATTACTGCTCTGGCTGAGAGATACGGCCACCCTGG | |
| | | | GGGACCGCGCGCGCCTGGGGCACCGGGAATAGGG | |
| | | | GGCAATCCCAATTCTCCCCCGATCCGTCGAGCCCG | |
| | | | CCACCCGGCGGCCGCTCCGGAGCCCCCAGCAGGT | |
| | | | AACCAGCCTCCGGCCCTGCCATGGCATGGGGATGG | |
| | | | TGGAAACGAAGGCGCAAGTGGTGGTGGAGACGACG | |
| | | | CTGGACTCGTGGCCGACTTCGCAAACGACGGGCTA | |
| | | | GACGAGCTGGTCGCCGCCCTCGACGAAGAAGAGTC | |
| | | | CCAAAAAACCCAGGGTCGACCTCGGGCCAATCCAA | |
| | | | CAGCAAGAAAGGCCCTCCGATTCACTCCAAAGAGAA | |
| | | | TCGAGGCCGTGGGAGACCAGCGAAGAAGAGAGCGA | |
| | | | AGCAGAAGTCCAGCAAGAAGAGACGGAGGAGGTGC | |
| | | | CCCTCAGACAGCAACTCCTCCACAACCTCAGAGAGC | |
| | | | AGCAGCAACTCCGAAAGGGCCTCCAGTGCGTCTTC | |
| | | | CAGCAGCTAATAAAGACGCAGCAGGGGGTTCACATA | |
| | | | GACCCATCCCTACTGTAGGCCCCAGTCAGTGGCTCT | |
| | | | TCCCCGAGAGAAAGCCTAAACCCCCTCCATCGGCC | |
| | | | GGAGACTGGGCCATGGAGTACCTAGCTTGCAAGAT | |
| | | | ATTCAACAGGCCGCCCCGCACTCACCTTACAGACCC | |
| | | | TCCTTTCTACCCCTACTGCAAAAACAATTACAATGTA | |
| | | | ACCTTTCAGCTCAACTACAAATAA | |
| GU797360.1 | AD051763.1 | ORF2 | ATGGCTGAGTTTATGCTGCCCGTCCGCAGAGAGGA | 646 |
| | | | GCCACGGCGGGGGATCCGAACGTCCCGAGGGCGG | |
| | | | GTGCCGGAGGTGAGTTTACACACCGCAGTCAAGGG | |
| | | | GCAATTCGGGCTCGGGACTGGCCGGGCTATGGGCA | |
| | | | AGGCTCTTAAAAAAGCCATGTTTCTCGGTAAATTACA | |
| | | | CAGAAAGAAGAGGGCACTGTCACTGCACGGCCTGC | |
| | | | CAGCTACAAAGAAAAAACCACCTCCTGATATGAACT | |
| | | | ACTGGAGGCCGCCTGTGCACAATGTCCCGGGGCTC | |
| | | | GAACGCCTCTGGTACGAGTCCGTGCATCGTAGCCAT | |
| | | | GCTGCTGTTTGTGGTTGTGGGGATTTTGTACGCCAT | |
| | | | ATTACTGCTCTGGCTGAGAGATACGGCCACCCTGG | |
| | | | GGGACCGCGCGCGCCTGGGGCACCGGGAATAGGG | |
| | | | GGCAATCCCAATTCTCCCCCGATCCGTCGAGCCCG | |
| | | | CCACCCGGCGGCCGCTCCGGAGCCCCCAGCAGGT | |
| | | | AACCAGCCTCCGGCCCTGCCATGGCATGGGGATGG | |
| | | | TGGAAACGAAGGCGCAAGTGGTGGTGGAGACGACG | |
| | | | CTGGACTCGTGGCCGACTTCGCAAACGACGGGCTA | |
| | | | GACGAGCTGGTCGCCGCCCTCGACGAAGAAGAGTT | |
| | | | GTTAGAGACCCCTGCACTCAGCCCACCTTCGAACTG | |
| | | | CCCGGAGCCAGTACGCAGCCTCCACGAATACAAGT | |
| | | | CACGGACCCGAAACTCCTCGGTCCCCACTACTCATT | |
| | | | CCACTCGTGGGACCTCAGACGTGGCTACTATAGCAC | |
| | | | AAAGAGTATTAAACGAATGTCAGAACACGAAGAACC | |
| | | | TTCTGAGTTTATTTTCCCAGGTCCCAAAAAACCCAGG | |
| | | | GTCGACCTCGGGCCAATCCAACAGCAAGAAAGGCC | |
| | | | CTCCGATTCACTCCAAAGAGAATCGAGGCCGTGGG | |
| | | | AGACCAGCGAAGAAGAGAGCGAAGCAGAAGTCCAG | |
| | | | CAAGAAGAGACGGAGGAGGTGCCCCTCAGACAGCA | |
| | | | ACTCCTCCACAACCTCAGAGAGCAGCAGCAACTCCG | |
| | | | AAAGGGCCTCCAGTGCGTCTTCCAGCAGCTAA | |
| GU797360.1 | AD051762.1 | ORF2 | ATGGCTGAGTTTATGCTGCCCGTCCGCAGAGAGGA | 647 |
| | | | GCCACGGCGGGGGATCCGAACGTCCCGAGGGCGG | |
| | | | GTGCCGGAGGTGAGTTTACACACCGCAGTCAAGGG | |
| | | | GCAATTCGGGCTCGGGACTGGCCGGGCTATGGGCA | |
| | | | AGGCTCTTAAAAAAGCCATGTTTCTCGGTAAATTACA | |
| | | | CAGAAAGAAGAGGGCACTGTCACTGCACGGCCTGC | |
| | | | CAGCTACAAAGAAAAAACCACCTCCTGATATGAACT | |
| | | | ACTGGAGGCCGCCTGTGCACAATGTCCCGGGGCTC | |
| | | | GAACGCCTCTGGTACGAGTCCGTGCATCGTAGCCAT | |
| | | | GCTGCTGTTTGTGGTTGTGGGGATTTTGTACGCCAT | |
| | | | ATTACTGCTCTGGCTGAGAGATACGGCCACCCTGG | |
| | | | GGGACCGCGCGCGCCTGGGGCACCGGGAATAGGG | |
| | | | GGCAATCCCAATTCTCCCCCGATCCGTCGAGCCCG | |
| | | | CCACCCGGCGGCCGCTCCGGAGCCCCCAGCAGGT | |
| | | | AACCAGCCTCCGGCCCTGCCATGGCATGGGGATGG | |
| | | | TGGAAACGAAGGCGCAAGTGGTGGTGGAGACGACG | |
| | | | CTGGACTCGTGGCCGACTTCGCAAACGACGGGCTA | |
| | | | GACGAGCTGGTCGCCGCCCTCGACGAAGAAGAGTA | |
| | | | A | |
| AB030487.1 | BAA90404.1 | ORF2a | ATGGCTGAGTTTTCCACGCCCGTCCGCAGCGAGAT | 648 |
| | | | CGCGACGGAGGAGCGATCGAGCGTCCCGAGGGCG | |
| | | | GGTGCCGAAGGTGAGTTTACACACCGGAGTCAAGG | |
| | | | GGCAATTCGGGCTCGGGACTGGCCGGGCTATGGGC | |
| | | | AAGGCTCTTAA | |
| AB030488.1 | BAA90407.1 | ORF2a | ATGGCTGAGTTTTCCATGCCCGTCCGCAGCGGTGAA | 649 |
| | | | GCCACGGAGGGAGCTCAGCGCGTCCCGAGGGCGG | |
| | | | GTGCCGAAGGTGAGTTTACACACCGAAGTCAAGGG | |
| | | | GCAATTCGGGCTCGGGACTGGCCGGGCTATGGGCA | |
| | | | AGGCTCTTAA | |
| AB030489.1 | BAA90410.1 | ORF2a | ATGGCTGAGTTTTCTATGCCCGTCCGCAGCGGCGAA | 650 |
| | | | GCCACGGAGGGAGCTCAGCGCGTCCCGAGGGCGG | |
| | | | GTGCCGGAGGTGAGTTTACACACCGAAGTCAAGGG | |
| | | | GCAATTCGGGCTCGGGACTGGCCGGGCTATGGGCA | |
| | | | AGGCTCTTAA | |
| AB030487.1 | BAA90405.1 | ORF2b | ATGCACTTTTCTAGGATATCCAGAAAGAAAAGGCTA | 651 |
| | | | CTGCTACTGCAAACAGTGCCAGCTCCACAGAAAACT | |
| | | | TTCAAACTTTTAAGAGGTATGTGGAGTCCTCCCACT | |
| | | | GACGATGAACGTGTCCGCGAGCGAAAATGGTTCCT | |
| | | | CGCAACTGTTTATTCTCACTCTGCTTTCTGTGGCTGC | |
| | | | AATGATCCTGTCGGTCACCTCTGTCGCTTGGCTACT | |
| | | | CTTTCTAACCGTCCGGAGAACCCGGGACCCTCCGG | |
| | | | GGGACGTCGTGCTCCTTCGATCGGGGTCCTACCCG | |
| | | | CTCTCCCGGCTGCTACCGAGCAGCCCGGTGATCGA | |
| | | | GCACCATGGCCTATGGGTGGTGGAGGAGACGCCGC | |
| | | | AGAAGGTGGAAGAGATGGAGGAGAAGGCCCAGGTG | |
| | | | GAGACGCCCATGGAGGACCCGCAGACGCAGACCTG | |
| | | | CTAGACGCCGTGGACGCCGCAGAACAGTAA | |
| AB030488.1 | BAA90408.1 | ORF2b | ATGCACTTTTCTAGGATACGCAGAAAGAAAAGGCTA | 652 |
| | | | CTGCTACTGCAAACAGTGCCAGCTCCACAGAAAACT | |
| | | | CTCAAACTTTTAAAAGGTATGTGGAGTCCTCCCACC | |
| | | | GACGATGAACGTGTCCGCGAGCGAAAATGGTTCCT | |
| | | | CGCAACTATTTATTCTCACTCTACTTTCTGTGGCTGC | |
| | | | AATGATCCTGTCGGTCACTTCTGTCGCCTGGCTACT | |
| | | | CTGTCTAACCGCCCGGAAAACCCGGGACCCTCCGG | |
| | | | AGGACGTAGTGCTCCTCAGATCGGGCTCCTACCCG | |
| | | | CTCTCCCGGCTGCTCCCGAGCAACCCGGTGATCGA | |
| | | | GCACCATGGCTTATGGGTGGTGGAGGAGACGCCGC | |
| | | | AGGAGGTGGAAGAGATGGAGGAGAAGGCCCAGGT | |
| | | | GGAGACGCCCATGGAGGACCCGCAGACGCAGACCT | |
| | | | GCTGGACGCCGTGGACGCCGCAGAACAGTAA | |
| AB030489.1 | BAA90411.1 | ORF2b | ATGCACTTTTCTAGGATACACAGAAAGAAAAGGCTA | 653 |
| | | | CTGCCACTGCAAACAGTGCCAACTCCACAGAAAACT | |
| | | | CTCAAACTTTTAAAAGGTATGTGGAGTCCTCCCACC | |
| | | | GACGATGAACGTGTCCGCGAGCGAAAATGGTTCCT | |
| | | | CGCAACTATCTATTCTCACTCTACTTTCTGTGGCTGC | |
| | | | AATGATCCTGTCGCTCATTTCTGTCGCCTGGCTACT | |
| | | | CTCTCTAACCGCCCGGAAAACCCGGGACCCTCCGG | |
| | | | AGGACGTAGTGCTCCTCAGATCGGGCTCCTACCCG | |
| | | | CTCTCCCGGCTGCTCCCGAGCAACCCGGTGATCGA | |
| | | | GCCCCATGGCCTATGGGTGGTGGAGGAGACGCCGC | |
| | | | AGGAGGTGGAAGAGATGGAGGAGAAGGCCCAGGT | |
| | | | GGAGACGCCGCTGGAGGACCCGCAGACGCAGACC | |
| | | | TGCTGGACGCCGTAGACGCCGCAGAACAGTAA | |
| AB038340.1 | BAA90824.1 | ORF2s | ATGTTTATTGGCAGGCATTACAGAAAGAAAAGGGCG | 654 |
| | | | CTGTCACTGTGTGCTGTGCGAACAACAAAGAAGGCT | |
| | | | TGCAAACTACTAATAGTAATGTGGACCCCACCTCGC | |
| | | | AATGATCAACAGTACCTTAACTGGCAATGGTACTCAA | |
| | | | GTGTACTTAGCTCCCACGCTGCTATGTGCGGGTGTC | |
| | | | CCGACGCTGTCGCTCATTTTAATCATCTTGCTTCTGT | |
| | | | GCTTCGTGCCCCGCAAAACCCACCCCCTCCCGGTC | |
| | | | CCCAGCGAAACCTGCCCCTCCGACGGCTGCCGGCT | |
| | | | CTCCCGGCTGCGCCAGAGGCGCCCGGAGATAGAG | |
| | | | CACCATGGCCTATGGCTGGTGGCGCCGAAGGAGAA | |
| | | | GACGGTGGCGCAGGTGGAGACGCAGACCATGGAG | |
| | | | GCGCCGCTGGAGGACCCGAAGACGCAGACCTGCTA | |
| | | | GACGCCGTGGCCGCCGCAGAAACGTAA | |
| AB038340.1 | BAA90826.1 | ORF3 | ATGTTTGGTGACCCCAAACCTTACAACCCTTCCAGT | 655 |
| | | | AATGACTGGAAAGAGGAGTACGAGGCCTGTAGAATA | |
| | | | TGGGACAGACCCCCCAGAGGCAACCTAAGAGACAC | |
| | | | CCCTTTCTACCCCTGGGCCCCCAAGGAAAACCAGTA | |
| | | | CCGTGTAAACTTTAAACTTGGATTTCAATAA | |
| AB038622.1 | BAA93587.1 | ORF3 | ATGATGAATATGTTGCAGGGCCTTTACCAAGAAAAA | 656 |
| | | | GAAACAAATTCGATACCAGAGCCCAAGGGCTGCAAA | |
| | | | CCCCCGAAAAAGAAAGCTACACTTTACTCCAAGCCC | |
| | | | TCCAAGAGTCGGGGCAAGAGACCAGCTCAGAAGAC | |
| | | | CAAGAACAAGCACCCCAAGAAAAAGAGGGTCAGAA | |
| | | | GGAAGCGCTCATGGAGCAGCTCCAGCTCCAGAAAC | |
| | | | AGCACCAGCGAGTCCTCAAGCGAGGCCTCAAACTC | |
| | | | CTCCTCGGAGACGTCCTCCGACTCCGGAGAGGAGT | |
| | | | CCACTGGGACCCCCTCCTGTCATAATTCAGGGCCCC | |
| | | | TCTATCCCAGACCTGCTTTTCCCTAA | |
| AB038623.1 | BAA93590.1 | ORF3 | ATGATGAATATGTTGCAGGGCCTTTACCAAGAAAAA | 657 |
| | | | GAAACAAGTTCGATACCAGAGCCCAAGGGCTCCAAA | |
| | | | GCCCCGAAAAAGAAAGCTACACTTTACTCCAAGCCC | |
| | | | TCCAAGAGTCGGGGCAAGAGAGCAGCTCAGAAGAC | |
| | | | CAAGAACAAGCACCCCAAGAAAAAGAGGGTCAGAA | |
| | | | GGAAGCGCTCATGGAGCAGCTCCAGCTCCAGAAAC | |
| | | | AGCACCAGCGAGTCCTCAAGCGAGGCCTCAAACTC | |
| | | | CTCCTCGGAGACGTTCTCCGACTCCGGAGAGGAGT | |
| | | | ACACTGGGACCCCCTCCTGTCATAATTCAGGGCCCC | |
| | | | TCTATCCCAGACCTACTTTTCCCTAA | |
| AB038624.1 | BAA93593.1 | ORF3 | ATGATGAATATGTTGCAGGGCCTTTACCAAGAAAAA | 658 |
| | | | GAAACAAGTTCGATACCAGAGCCCAAGGGCTCCAAA | |
| | | | GCCCCGAAAAAGAAAGCTACACTTTACTCCAAGCCC | |
| | | | TCCAAGAGTCGGGGCAAGAGACGAGCTCAGAAGAC | |
| | | | CAAGAACAAGCACCCCAAGAAAAAGAGGGTCAGAA | |
| | | | GGAAGCGCTCATGGAGCAGCTCCAGCTCCAGAAAC | |
| | | | AGCACCAGCGAGTCCTCAAGCGAGGCCTCAAACTC | |
| | | | CTCCTCGGAGACGTTCTCCGACTCCGGAGAGGAGT | |
| | | | ACACTGGGACCCCCTCCTGTCATAATTCAGGGCCCC | |
| | | | TCTATCCCAGACCTGCTTTTCCCTAA | |
| AB050448.1 | BAB19926.1 | ORF3 | ATGAGCTTTGTAGAACCCTTACTAACCAGCACCCAC | 659 |
| | | | AGAGAGATAGCATACTACCATGGCTGTGTTCAGATG | |
| | | | CACAAAGCCTTCTGTGGGTGTGACAACTTTCTTACC | |
| | | | CACCTGCAACGCATAACAACATACATCTCTGCTAAC | |
| | | | CAACACACTCCACCCAGCACACCCTCAAACACCCTC | |
| | | | CGTAGAGCCCGGGCCCTGCCCGCGGCTCCGGAGC | |
| | | | CAGCTCCATGGCGTGGACCTGGTGGTGGCAGAGGA | |
| | | | GGCGCCGAAGGTGGCCGTGGAGAAGGAGAAGGTG | |
| | | | GAGAAGACTACGCACAAGAAGACCTAGACGCCTTGT | |
| | | | TCGACGCCGTCGCAAGAGATACAGAGTTATCAGAAA | |
| | | | CCCTTGTAAAACAGAAGGACACGATCTCCCTCACAC | |
| | | | CAGTAGACTCCATCGCGACTTACAAGTTGTTGACCC | |
| | | | ACACACCGTGGGCCCCCAATGGGCGCTCCACACCT | |
| | | | GGGACTGGCGACGTGGACTCTTTGGTTCAGAGGCT | |
| | | | ATCAAAAGAGTGTCTGAACAACAAGTACATGATGAA | |
| | | | CTGTATTACCCACCTTCAAAGAAACCTCGATTCCTCC | |
| | | | CTCCAATATCAGGCCTCCAAGAGCAAGAAAGAGACT | |
| | | | ACAGTTCGCAGGAGGAGAAAGAACAGTCCTCCTCA | |
| | | | GAAGAAGAGACGGACCCGAAGAAAAAAGAGCAAAA | |
| | | | ACAGCAGCAGCGACTCCACCTCCAGTTCCAAGAGC | |
| | | | AGCAGCGACTCGGAAACCAACTCCGACTCATCTTCC | |
| | | | GAGAGCTACAGAAAACCCAAGCGGGTCTCCACTTAA | |
| AF371370.1 | AAK54733.1 | ORF3 | ATGGCGTGGTCGTGGTGGTGGAGGCGAAGGAAACG | 660 |
| | | | CTGGTGGCCGCGCAGAAGGAGGCGATGGAGAAGG | |
| | | | CTACGAACCCGAAGAACTGGAAGAGCTGTTCCGCG | |
| | | | CCGCCGCCGCCGACGACGAGTAAGGAGGCGCCGG | |
| | | | TGGGGGAGGCGACCGCGTAGGAGACGGGTGTACTA | |
| | | | TAAGAGACGCAGACGAAAGACTGGCAGACTGTATAG | |
| | | | AAAGCCTAAAAAAAAACTAGTACTGACTCAATGGCA | |
| | | | CCCCACTACAGTTAGAAACTGCTCCATACGGGGCTT | |
| | | | AGTGCCCCTAGTCCTCTGCGGACACACACAGGGAG | |
| | | | GCAGAAACTTTGCTTTGAGGAGCGATGACTACCCCA | |
| | | | AACAAGGCACCCCATACGGGGGCAGCTTCAGCACT | |
| | | | ACAACCTGGAACCTCAGGGTGCTTTTCGACGAGCAC | |
| | | | CAAAAACACCACAATACGTGGAGCTATCCAAGCAAT | |
| | | | CAACTAGACCTAGCCAGATTTAGAGGCAGCATATTT | |
| | | | TACTTTACAGAGACAAAAAAACTGACTACATAG | |
| AB060596.1 | BAB69914.1 | ORF3 | ATGAGCTGGTGTACTCCAGTTGAAAATGCCTATAAG | 661 |
| | | | AGAGAGATCCACTTTCTCAGGGGCTGTCAACTGCTT | |
| | | | CACACTAGCTTTTGTGGTTGCGATGATTTTATTAATC | |
| | | | ATATTATTCGCCTACAAAATCTTCACGGCAACCTACA | |
| | | | CCAGCCCACGGGACCGTCCACACCTCCAGTGACCC | |
| | | | GTAGAGCTCTGGCCTTGCCGGCTGCTCCGGAGTCA | |
| | | | TGGCGTTCCGGTGGTGGTGGTGGAGACGCCGCCC | |
| | | | GCAGCGACGATGGACCCGGCGCCGATGGAGGAGA | |
| | | | CTACGAACCCGCCGACCTAGACGCACTGTACGACG | |
| | | | CCGTCGCCGCAGACCAAGAATTATCAAAAACCCGTG | |
| | | | TAAAAAAGAAGAATCCACATTCACCTATCCCAGTAGA | |
| | | | GAGCCTCGCGACCTACAAGTTGTTGACCCACTCACC | |
| | | | ATGGGCCCAGAATGGGTCTTCCACACATGGGACTG | |
| | | | GAGACGTGGACTTTTTGGTAAAAATGCTGTCGACAG | |
| | | | AGTGTCAAAAAAACCAGACGATGATGCAGAATATTAT | |
| | | | CCAGTACCAAAAAGGCCTCGATTCTTCCCTCCAACA | |
| | | | GACACACAGTCAGAGCCAGAAAAAGACTTCGGTTTC | |
| | | | ACACCGGAGAGCCAAGAGTTACAGCAAGAAGACTTA | |
| | | | CGAGCACCCCAAGAAGAAAGCCAAGAGGTACAGCA | |
| | | | GCAGCGACTGCTCCAGCTCAGACTCTCACAGCAGTT | |
| | | | CAGACTCAGACAGCAGCTCCAGCACCTGTTCGTACA | |
| | | | AGTCCTCAAAACCCAAGCAGGTCTCCACATAA | |
| AB060592.1 | BAB69898.1 | ORF3 | ATGAGCTTTGTAGAACCGTTACTAAGCAGCACCCAC | 662 |
| | | | CGAGAGATAGCATTCTACCATGGCTGTGTTCAAATG | |
| | | | CACAAGGCCTTCTGTGGCTGTGACAACTTTCTTACC | |
| | | | CACCTGCAGCGCATAACAACATACATCTCTGCTAAT | |
| | | | CAACACACTCCACCCAGCACACCCTCAAACACCCTC | |
| | | | CGTAGAGCCCGGGCCCTGCCCGCGGCTCCGGAGC | |
| | | | CAGCTCCATGGCGTGGACCTGGTGGTGGCAGAGGA | |
| | | | GGCGCCGAAGGTGGCCGTGGAGAAGGAGAAGGTG | |
| | | | GAGAAGACTACGCACCAGAAGACCTAGACGACTTGT | |
| | | | TCGCCGCCGTCGCAAGAGATACAGAGTTATCAGAAA | |
| | | | CCCTTGTAAAACAGAAGGACACGATCTCCCTCACAC | |
| | | | CAGTAGACTCCATCGCGACTTACAAGTTGTTGACCC | |
| | | | ACACACCGTGGGCCCCCAATGGGCGCTCCACACCT | |
| | | | GGGACTGGCGACGTGGACTCTTTGGTTCAGAGGCT | |
| | | | ATCAAAAGAGTGTCTGAACAACAAGTACATGATGAA | |
| | | | CTGTATTACCCAGCTTCAAAGAAACCTCGATTCCTCC | |
| | | | CTCCAATATCAGGCCTCCAAGAGCAAGAAAGAGACT | |
| | | | ACAGTTCGCAGGAGGAAAAAGACCAGTCCTCCTCAG | |
| | | | AAGAAGAGAAGGACCCGAAGAAAAAAGAGCAAAAAC | |
| | | | AGCAGCAGCGACTCCACCTCCAGTTCCAAGAGCAG | |
| | | | CAGCGACTCGGAAACCAACTCCGACTCATCTTCCGA | |
| | | | GAGCTACAGAAAACCCAAGCGGGTCTCCACATAA | |
| AB060593.1 | BAB69902.1 | ORF3 | ATGAGTCTGTGGCGACCCCCGGTCCACAATGCCCC | 663 |
| | | | CGGCAGAGAGAGACTTTGGTTTCAGGCCTGTTACGA | |
| | | | ATCTCACAGTGCTTTTTGTGGCTGTGGTAGCTTTATT | |
| | | | CTTCATCTTACTAGCTTGGCTGCACGTTTTAATTTTC | |
| | | | AGGCCGGGCCACCGCCTCCCGGGGGTCCCCGGGC | |
| | | | GGAGACCCCGCCGATTCTGAGGGCGCTGCCGGCAC | |
| | | | CCCAGCCGCGCCGCCACCGCCAGACGGAGAACCC | |
| | | | CGGGTCTGAGCCATGGCCTGGAGATGGTGGTGGAG | |
| | | | ACGGCGCTGGAAGCCAAGAAGGCGGCCAGCGTGG | |
| | | | ACCAAGTACCGCAGACGCAGGTGGAGACGACTTCG | |
| | | | ACCCCGCAGACCTAGAAGACTTGCTCGCGGCCGTC | |
| | | | GAAGAAGACGAACAGTCATCAAAGACCCGTGCAGCT | |
| | | | CCTCAGGACTGGCACCTACCGACTCCAGTAGATTCA | |
| | | | AGCGGGATGTACAAGTCGTTAGCCCGCTCACAATG | |
| | | | GGGCCCCGACTGCTATTCCACTCGTTCGACCAAAGA | |
| | | | CGAGGGTTCTTTACTCCAGGAGCTATCAAACGAATG | |
| | | | CATGATGAACAAATTAATGTTCCAGACTTTACACAAA | |
| | | | AACCTAAAATCCCGCGAATTTTCCCACCAGTCGAGC | |
| | | | TCCGAGAAAGAGCAGAAGCCGAAGAAGACTCAGGT | |
| | | | TCGGAAAAAGCGTCGTTCACCTCGTCGCAAGAGAGA | |
| | | | GAAGCCGAAGCCCAAGAAAAGTTACCGATACAGCTC | |
| | | | CAGCTCAGACAGCAGCTCAGACAACAACAGCAGCT | |
| | | | CCGAGTCCACTTGCAGCAAGTCTTCCTCCAACTCCA | |
| | | | AAAAACGAAGGCACATTTACATATAA | |
| AB060595.1 | BAB69910.1 | ORF3 | ATGAATCTCTGGCGACCCCCTCTGAGAAATATCCCC | 664 |
| | | | CACAGGGAGAGATGTTGGCTTGAGGCCTGTCTCAG | |
| | | | AGCCCACGATTCTTTTTGTGGCTGTCCTAGTCCTATT | |
| | | | GTTCATTTTTCTAGTCTGGTTGCACGTTTTAATCTAC | |
| | | | AAGGAGGCCCGCCGCCAGAGGATGACTCCCCACAG | |
| | | | GGCGCGCCAGTCCTGAGGGCCCTGCCGGCACCGA | |
| | | | GCCCCCACAGGCACACCCGCACGGAGAACCCCTCC | |
| | | | GGTGAGCCATGGCCTACTCCTACTGGTGGCGCCGC | |
| | | | CGGAGGTGGCCGTGGAGAGGCCGATGGAGGCGCT | |
| | | | GGAGGCGCCGCAGACGAATACCGCGCCGAAGACCT | |
| | | | AGACGACCTGTTCGCCGCTATCGAAGGAGACCAAC | |
| | | | GATCAGAAACCCGTGCACCTCGGACGGACAGACGC | |
| | | | CCACAACCAGTAGACAGTCTAGAGAGGTACAAATCG | |
| | | | TTGACCCGCTCACCATGGGACCCCGATACGTATTCC | |
| | | | ACTCGTGGGACTGGCGACGTGGGTGGCTTAATGAC | |
| | | | AGAACTCTCAAACGCTTGTTCCAAAAACCGCTCGAT | |
| | | | TTTGAAGAGTATCCAAAATCTCCAAAGAGACCTAGAA | |
| | | | TTTTCCCACCCACAGAGCAGCTCCAAGAAGACCCGC | |
| | | | AAGAGCAAGAAAGAGACTCCTCTTCTTCGGAAGAAA | |
| | | | GTCTCCCTACATCGTCAGAAGAGACACCGCCAGCC | |
| | | | CACCTACTCAGAGTACACCTCAGAAAGCAGCTCCGG | |
| | | | CAACAGCGAGACCTCCGAGTCCAGCTCAGAGCCCT | |
| | | | GTTCGCCCAAGTCCTCAAAACGCAAGCGGGCCTAC | |
| | | | ACATAA | |
| AB064596.1 | BAB79312.1 | ORF3 | ATGCCGTGGAGACCGCCGGCTCATAACGTCCAGGG | 665 |
| | | | GCGAGAGAGCCAGTGGTTCGCGGCTTGTTTTCACG | |
| | | | GCCACGCTTCGTTTTGCGGCTGCGGTGACTTTATTG | |
| | | | GGCATATTAACAGCCTTGCTCCTCGCTTTCCTAACAA | |
| | | | CCAAGGACCCCCGCATCCACCTGCCTTAAACAGGC | |
| | | | CACCTGCACAGGGCCCAGAAAGCCCCGGGGGTTCC | |
| | | | ATACTACCCCTGCCAGCCCTACCGGCACCACCTGAT | |
| | | | CCGCCACCACGGCCTGGTGGTGGGGAAGACGGTG | |
| | | | GCGACGCCGCCCGTGGGGCCGCTGGCGCCGCCGA | |
| | | | AGGCGCGTATGGAGAAGAAGACCTAGAACTGCTGTT | |
| | | | CGCCGCCGCCGAGGAAGACGATATGCAATCGACGA | |
| | | | CCCCTGCCAGCAGGGAACCCACCCGCTTCCCGAGC | |
| | | | CCGGTACGTTGCCTAGAATCTTACAAGTCAGCGACC | |
| | | | CGACGCAACTCGGACCGAAAACCATATTCCACCTCT | |
| | | | GGGACCAGAGGCGTGGACTTTTTAGCAAAAGAAGTA | |
| | | | TTGAAAGAATGTCAGAATACAAAGGAACTGATGACTT | |
| | | | ATTTTCACCAGGTCGCCCAAAGCGCCCAAAGCTCGA | |
| | | | CACACGTCCCGAAGGACTACCAGAGGAGCAAAGAG | |
| | | | GAGCTTACAATTTACTCCAAGCCCTCGAAGACTCAG | |
| | | | CCCAGTCGGAAGAAAGCGACCAAGAAGAAATGCCT | |
| | | | CCCCTCGAAGAAGAACAAGTACTCCACGAGCAAAAG | |
| | | | AAAGAGGCGCTCCTCCAGCAGCTCCAGCAGCAGAA | |
| | | | ACACCACCAGCGAGTCCTCAAGCGAGGCCTCAGAC | |
| | | | TCCTCCTCGGAGACGTCCTGA | |
| AB064597.1 | BAB79316.1 | ORF3 | ATGCCGTGGAGACCGCCGGTGCATAGTGTCCAGGG | 666 |
| | | | GCGAGAGGATCAGTGGTTCGCGAGCTTTTTTCACGG | |
| | | | CCACGCTTCATTTTGCGGTTGCGGTGACGCTGTTGG | |
| | | | CCATCTTAATAGCATTGCTCCTCGCTTTCCTCGCGC | |
| | | | CGGTCCACCAAGGCCCCCTCCGGGGCTAGAGCAGC | |
| | | | CTAACCCCCCGCAGCAGGGCCCGGCCGGGCCCGG | |
| | | | AGGGCCGCCCGCCATCTTGGCGCTGCCGGCTCCGC | |
| | | | CCGCGGAGCCTGACGACCCGCAGCCACGGCGTGG | |
| | | | TGGTGGGGACGGTGGCGCCGCCGCTGGCGCCGCA | |
| | | | GGCGACCGTGGAGACCGAGACTACGACGAAGAAGA | |
| | | | GCTAGACGAGCTTTTCCGCGCCGCCGCCGAAGACG | |
| | | | ATTTGGAACCCACCCGATTCCCGACCCCGATAAGCA | |
| | | | CCCTCGCCTCCTACAAGTGTCGAACCCGAAACTGCT | |
| | | | CGGACCGAGGACAGTGTTCCACAAGTGGGACATCA | |
| | | | GACGTGGGCAGTTTAGCAAAAGAAGTATTAAAAGAG | |
| | | | TGTCAGAATACTCATCGGATGATGAATCTCTTGCGC | |
| | | | CAGGTCTCCCATCAAAGCGAAACAAGCTCGACTCGG | |
| | | | CCTTCAGAGGAGAAAACCCAGAGCAAAAAGAATGCT | |
| | | | ATTCTCTCCTCAAAGCACTCGAGGAAGAAGAGACCC | |
| | | | CAGAAGAAGAAGAACCAGCACCCCAAGAAAAAGCC | |
| | | | CAGAAAGAGGAGCTACTCCACCAGCTCCAGCTCCA | |
| | | | GAGACGCCACCAGCGAGTCCTCAGACGAGGGCTCA | |
| | | | AGCTCGTCTTTACAGACATCCTCCGACTCCGCCAGG | |
| | | | GAGTCCACTGGAACCCCGAGCTCACATAGAGCCCC | |
| | | | CACCTTACATACCAGACCTACTTTTTCCCAATACTGG | |
| | | | TAA | |
| AB064599.1 | BAB79324.1 | ORF3 | ATGCCGTGGTCTCTGCCGAGACATAATATCAGAACG | 667 |
| | | | AGAGAAGATCTCTGGGTGCAATCGATTCTTTATTCAC | |
| | | | ATGACACTTTTTGTGGCTGTGATAATATTCCTGAGCA | |
| | | | TCTTACTGGCCTCCTGGGCGGCGTACGACCAGCTC | |
| | | | CACCTAGAAACCCAGGACCCCCTACCATACGGAGC | |
| | | | CTGCCGGCACTGCCGCCAGCTCCGGAACCCCCTGA | |
| | | | GGAACCACGGCGTGGTGGAGATACAGACGGAGACC | |
| | | | GTGGAGAAGATGGAGGAGACGCCGCTGGGGCCTAC | |
| | | | GAACCCGAAGACCTAGAAGAACTTTTCGCCGCCGC | |
| | | | CGAGCAAGACGATATCCCATTGACGACCCCTGCCAA | |
| | | | AAAGGAAAACACGACATTCCCGACCCCGATACAAAC | |
| | | | CCTCCAAGAATACAAATATCAGACCCGCAACACCTC | |
| | | | GGACCGGCGACGCTGTTCCACTCGTGGGACCTCAG | |
| | | | ACGTGGATATATTAATACAAAAAGTATTAAAAGAATC | |
| | | | TCAGAACACCTCGATGCTAATGAATATTTTTCGACAG | |
| | | | GCGTCGTGTCCAAAAAACCCCGATTCGACACTCCCC | |
| | | | ACCACGGGCAGCTATCAAACCAAGAAGAAGACGCC | |
| | | | TTGTCTATCCTCAGACAACCCCAAAAAGAGCAAGAA | |
| | | | GAGACCACCTCCGAGGAAGAACAAGCACTCCAAAAA | |
| | | | GAAGAGGAGCAAAAAGAAAAGCTCCTACAGCAACTC | |
| | | | AGAGTCCAGCGACAGCACCAGCGAGTCCTCAGACA | |
| | | | GGGAATCAAACACCTCATGGGAGACGTCCTCCGACT | |
| | | | CAGACAGGGAGTCCACTGGAACCCAGTCCTATAATA | |
| | | | CTTCCACCAGAACCAATACCAGACCTCTTATTCCCC | |
| | | | AATACTGGTAA | |
| AB064600.1 | BAB79328.1 | ORF3 | ATGTCGTGGAGACCGCCGAGCCAAAATTTACTGCAA | 668 |
| | | | AGAGAAGAGGCCTGGTACTCAGCTTTTCTTAGCTCG | |
| | | | CATTCTACATTTTGCGGTTGTACTGACCCTCTGCTGC | |
| | | | ATATTACTCTCATTGCTGGCCGCCTTACTAACCCCGT | |
| | | | ACCCGTCACCCGCCAACCGGAGACCCCTCCTAACG | |
| | | | GCCTCAGGGGGCTGCCGGCACTGCCAGCACCCCCT | |
| | | | GAACCACCAGCACCGCCACCACGGCCTGGGGATGG | |
| | | | TACCGGAGAAGAAGATGGCGCCCATGGAGAAGGAG | |
| | | | AAGGTGGGCGATACGCAGAAGAAGACCTAGAAGAA | |
| | | | CTGTTCGCCGCCGCGGCAGAAGACGATATCCTATC | |
| | | | GACGACCCCTACCAAAAACCCACCCACGAAATACCC | |
| | | | GACCCCGATAAGCACCCTCCAAGACTACAAATTGCA | |
| | | | GACCCGAAAATCCTCGGACCGTCGACAGTCTTCCAC | |
| | | | ACATGGGACATCAGACGTGGCCTCTTTAGCACAGCA | |
| | | | AGTCTTAAGAGAGTGTCAGAATACCAACCGCCTGAT | |
| | | | GACCTTTTTTCAACAGGCGTCGCATCCAAAAGACCC | |
| | | | CGATTCGACACTCCAGTCCAAGGGCAGCTCGAAAG | |
| | | | CCAAGAAGAAGAAAGCTATCGTTTACTCAGAGCACT | |
| | | | CCAAAAAGAGCAAGAGACAAGCAGCTCGGAAGAGG | |
| | | | AGCAGCCACAAAACCAAGAGATCCAAGAAAAACTAC | |
| | | | TCCTCCAGCTCCAGCAGCAGCGACAACAGCAGCGA | |
| | | | CTCCTCGCAAAGGGAATCAAGCACCTCCTCGGAGAT | |
| | | | GTCCTCCGACTCCGAAAAGGAGTCCACTGGGACCC | |
| | | | GGTCCTTACATAGCACCTCCAGAACCTATCCCAGAC | |
| | | | CTTTTGTTCCCCAGTACTAA | |
| AB064601.1 | BAB79332.1 | ORF3 | ATGTCGTGGGCTCCGCCGCTATTCAACTCGAAACAG | 669 |
| | | | AGAGAGGACCAGTGGTACCAGTCAATTATTTTCAGC | |
| | | | CATAATACTTTTTGCGGCTGCGGTGACCTTGTTAGG | |
| | | | CATTTTTGCGTCGTTGCTTCTCGCTTTACTGAGCCTC | |
| | | | CTGTAGTGCCGGCCCTACCGGCACCGGTACCGGCA | |
| | | | CCGCCACGGCGTGGTACAGAAGAAGAAGGTGGAGA | |
| | | | CCGTGGAGAAGACGCCGCAGACCGTGGACCCTACG | |
| | | | CAGAAGAAGAGCTAGAAGATTTGTTCGCCGCCGCC | |
| | | | CGAGAAGACGATATCCCATCGACGACCCCTGCCAAA | |
| | | | AAGACACCCACGAAATACCCGACCCCGATAAACACC | |
| | | | CTAGAGGAATACAAATATCAGACCCGAAGGTACTCG | |
| | | | GACCACCCACAGTCTTCCACACATGGGACATCAGAC | |
| | | | GTGGACTGTTTAGCTCGACGAGTCTTAAAAGAGTGT | |
| | | | CAGAATACCAACCGCCTGATGACCCTTTTTCAACAG | |
| | | | GCGTCGTCTTCAAAAGACCCCGACTGGAAACCCAGT | |
| | | | ACAAAGGAACCCAAGAAACCCCAGAAGAAGACGCC | |
| | | | TACACTTTACTCAAAGCACTCCAAAAAGAGCAAGAG | |
| | | | AGCAGCAGCTCGGAAGAAGAACTCCCACAAGAAGA | |
| | | | GCAAGAGATCCAAAAAACACAACTCCTCAAGCAGCT | |
| | | | CCAACTCCAGCAGCAGCAACAGCGAATCCTCAAGA | |
| | | | GGGGAATCAGACACCTCTTCGGAGACGTCCTCCGA | |
| | | | CTCAGAAAAGGAGTCCACTCCAACCCAGACCTATTA | |
| | | | TAATACCAGCAGAGGAAATCCCAGACCTGCTTTTCC | |
| | | | CCAATACTGGTAA | |
| AB064602.1 | BAB79336.1 | ORF3 | ATGCCGTGGCATCCACCGGGCTACAACGTTCAACA | 670 |
| | | | GAGAGAAGAGCTCTGGGTACAGACAGTTACTACTTC | |
| | | | ACATGCTACTTTTTGCGGCTGTGGTGACCCTAGTAG | |
| | | | CCATCTTCACCGCATTCTTAGCCGCCTTAATAACAG | |
| | | | CAGCCGGCGGCCCCCCGAAACCCCAAACCCCATTC | |
| | | | GTGCCCTACCGGCCCTACCGGCACCCCAAGAACCT | |
| | | | GAACAGCCGCCATCACGGCCTGGTACCGGTACAGA | |
| | | | AGAAGGCCATGGCGCCGAAGGAGGCGACCGAGGT | |
| | | | GGGGCCTACGCAGAAGAAGATTTAGAAGATCTTTTC | |
| | | | GCGGCCGCGGAAGAAGACGATATCCCATCGACGAC | |
| | | | CCATGCCAAAAGCCCACCCACGACCTTCCCGACCC | |
| | | | CGATAGACACCCCCCAAGAATACAAATCTCGGACCC | |
| | | | GGCAAGACTCGGACCGGAGACGCTCTTCCACTCAT | |
| | | | GGGACATCAGACGTGGATACATTAACACAAAAGCTA | |
| | | | TTAAAAGAATCTCAGATTACACAGAATCTAATGACTA | |
| | | | TTTTTCAACAGGCGTCGTGTCAAAAAGACCCCGATT | |
| | | | GGAAACCCAGTACCACGGCCAACACGAAAGCCAAG | |
| | | | AAGAAGACGCCTATCTTTTACTCAAACAACTCCAGG | |
| | | | AAGAGCAAGAAACGAGCAGTTCGGAGGGAGAACAA | |
| | | | GCACCCCAAGAAAAAACACTCCAAAAAGAAAAGCTC | |
| | | | CTCAAGCAGCTGCAGCTCCACAAGCAGCAGCAGCA | |
| | | | ACTCCTCAGAAAAGGAATCAGACACCTCCTCGGGGA | |
| | | | CGTCCTCCGACTCAGACGGGGAGTCCACTGGGACC | |
| | | | CAGGCCTATAGTACTGCCTCCAGAGCCTATTCCAGA | |
| | | | CTTGCTTTTCCCAAATACTAA | |
| AB064603.1 | BAB79340.1 | ORF3 | ATGTCGTGGCGACCGCCGTTGCATTCTATCCAAGGC | 671 |
| | | | AGAGAAGATCAATGGTATGCAGGCATCTTTCATACG | |
| | | | CATTTTGCTTTTTGCGGTTGTGGTGACCCTGTTGGG | |
| | | | CGTATTAACCGCATTGCTCACCGCTTTCCTAACGCC | |
| | | | GGTCCCCCGAGACCACCTCCAGGGCTAGACCAGCC | |
| | | | CAACCTCGGAGGGCCGGAAGGTCCAGGAGGTGCC | |
| | | | CCTAGAGCCCTGCCAGCCCTGCCGGCCCCGGCAGA | |
| | | | GCCAGAGCCGGCACCACGGCGTGGTGGTGGGGCC | |
| | | | GATGGAGACAGCGCCGCTGGGGCCGCCGCCGCCG | |
| | | | CAGACCATGGAGGGTACGACGAAGGAGACCTAGAA | |
| | | | GATCTTTTCGCCGCCGCCGCCGAGGACGATATGCA | |
| | | | ATCGACGACCCCTGCCAGAAGCCCACCCATGAGCT | |
| | | | ACCCGATCCCGATAGACACCCTCGCATGTTACAAGT | |
| | | | CTCTGACCCGACAAAGCTCGGACCGAAGACAGTGTT | |
| | | | CCACAAATGGGACTGGAGACGTGGGCAACTTAGCA | |
| | | | AAAGAAGTATTAAAAGAGTCCAAGAAGACTCAACGG | |
| | | | ATGATGAATATGTTACAGGGCCTTTATCAAGAAAAAG | |
| | | | AAACAAGCTCGACACAAAGATGCCAGGCCCCCCAA | |
| | | | CCCCCGAAAAAGAAAGCTACACTTTACTCCAAGCCC | |
| | | | TCCAAGAGTCGGGCCAGGAGAGCAGCTCCCAGGAC | |
| | | | GAAGAACAAGCACCCCAAAAAGAAGAGAACCAGAAA | |
| | | | GAAGCGCTCGTGGAGCAGCTCCAGCTCCAGAAACA | |
| | | | GCACCAGCGAGTCCTCAAGCGAGGCCTCAAACTCC | |
| | | | TCTTGGGAGACGTCCTCCGACTCCGCCGCGGAGTC | |
| | | | CACTGGGACCCCCTCCTATCCTAATTCAGGGTCCCT | |
| | | | CTATCCCAGACCTGCTTTTCCCTAA | |
| AB064604.1 | BAB79344.1 | ORF3 | ATGAGTATTTGGAGGCCTCCACTGCACAATGTCCCG | 672 |
| | | | GGACTCGAACACCTCTGGTACGAGTCAGTGCATCGT | |
| | | | AGCCATGCTGCTGTTTGTGGCTGTGGGGATCCTGTA | |
| | | | CGCCATCTTACTGCTCTTGCTGAAAGATATGGCATT | |
| | | | CCGGGAGGGTCGCGGTCTTCTGGGGCACCGGGAG | |
| | | | TAGGGGGCAACCACAACCCTCCCCAGATCCGTCGA | |
| | | | GCCCGCCACCCGGCGGCTGCTCCGGACCCCCCAG | |
| | | | CAGGTAACCAGCCTCCGGCCCTGCCATGGCATGGG | |
| | | | GATGGTGGAAACGAAAGCGGCGCTGGTGGTGGAGA | |
| | | | AAGCGGTGGACCCGTGGCCGACTTCGCAGACGATG | |
| | | | GCCTAGACGATCTCGTCGCCGCCCTCGACGAAGAA | |
| | | | GAATTGTTAAAGACCCCTGCACCCAGCCCACCTTTG | |
| | | | AAATACCCGGTGGCGGTAACATCCCTCGCAGAATAC | |
| | | | AAGTCATCAATCCGAAAGTCCTCGGACCCAGCTACA | |
| | | | GTTTCAGATCCTTTGACCTCAGACGTGACATGTTTAG | |
| | | | CGGCTCGAGTCTTAAAAGAGTCTCAGAACAACAAGA | |
| | | | GACTTCTGAGTTTTTATTCTCCGGCGGCAAACGCCC | |
| | | | CAGGATCGACCTTCCCAAGTACGTCCCGCCAGAAG | |
| | | | AAGACTTCAATATCCAAGAGAGACAACAAAGAGAAC | |
| | | | AGAGACCGTGGACGAGCGAAAGCGAGAGCGAAGCA | |
| | | | GAAGCCCAAGAAGAGACGCAGGCGGGCTCGGTCC | |
| | | | GAGAGCAGCTCCAGCAGCAGCTCCAAGAGCAGTTT | |
| | | | CAACTCCGAAGAGGGCTCAAGTGCCTCTTCGAGCA | |
| | | | GTTAG | |
| AB064606.1 | BAB79352.1 | ORF3 | ATGAGCTTCTGGAGACCTCCGGTGCACAATGCCAC | 673 |
| | | | GGGGATCCAGCGCCTGTGGTACGAGTCCTTTCACC | |
| | | | GTGGCCATGCTGCTTTTTGTGGTTGTGGGGATCCTA | |
| | | | TACTTCACATTACTGCACTTGCTGAGACATATGGCCA | |
| | | | TCCAACAGGCCCGAGACCTTCTGGGCCACCGCGAG | |
| | | | TAGACCCCGATCCCCAGATCCGTAGAGCCAGGCCT | |
| | | | GCCCCGGCCGCTCCGGAGCCCTCACAGGTTGAGCC | |
| | | | GAGACCTGCCCTGCCATGGCATGGGGATGGTGGAA | |
| | | | GCGACGGCGGCGCTGGTGGTTCCGGAAGCGGTGG | |
| | | | ACCCGTGGCAGACTTCGCAGACGATGGCCTCGATC | |
| | | | AGCTCGTCGCCGCCCTAGACGACGAAGAATTGTACA | |
| | | | AGATCCCTGCACACAGTCCACCTATGACATCCCCGG | |
| | | | CACCGGTAACTTGCCTCGCAGAATACAAGTCATTGA | |
| | | | CCCGAAAGTCCTCGGTCCCCACTACTCATTCCACCG | |
| | | | CTGGGACTTCAGGCGTGGCCTCTTTGGCCAACAAG | |
| | | | CTATTAAGAGAGTGTCAGAACAACCAACAACTTCTG | |
| | | | AGTTTTTATTCTCAGGTCCAAAGAGACCCAGAATCG | |
| | | | ATCAAGGGCCTTACATCCCGCCAGAAAAAGGCTCAG | |
| | | | ATTCACTCCAAAGAGAATCGAGACCGTGGAGCAACT | |
| | | | CGGAGACCGAGGCAGAGACAGAAGCCCCCTCGGAA | |
| | | | GAAGAGCCGGAGAACCAAGAAGAACAAGTACTCCA | |
| | | | GTTGCAGCTCCGACAGCAGCTCCGAGAACAGCGAA | |
| | | | AACTCAGACAGGGAATCCAGTGCCTCTTCGAGCAAC | |
| | | | TGA | |
| FJ426280.1 | ACK44073.1 | ORF3 | ATGCTATCCAGAGAGTGTCACAAAAACCGGAAGATG | 674 |
| | | | CTCTCCGCTTTACAAACCCTTTCAAGAGACCCAGAT | |
| | | | ATCTTCCCCCGACAGACGGAGAAGACTACCGACAA | |
| | | | GAAGAAGACTTCGCTTTACAGGAAAGAAGACGGCG | |
| | | | CACATCCACAGAAGAAGTCCAGGACGAGGAGAGCC | |
| | | | CCCCGCAAAACGCGCCGCTCCTACAGCAGCAGCAG | |
| | | | CAGCAGCGGGAGCTCTCAGTCCAGCACGCGGAGCA | |
| | | | GCAGCGACTCGGAGTCCAACTCCGATACATCCTCCA | |
| | | | AGAAGTCCTCAAAACGCAAGCGGGTCTCCACCTAA | |
| AB050448.1 | BAB19925.1 | ORF4 | ATGAGCTTTGTAGAACCCTTACTAACCAGCACCCAC | 675 |
| | | | AGAGAGATAGCATACTACCATGGCTGTGTTCAGATG | |
| | | | CACAAAGCCTTCTGTGGGTGTGACAACTTTCTTACC | |
| | | | CACCTGCAACGCATAACAACATACATCTCTGCTAAC | |
| | | | CAACACACTCCACCCAGCACACCCTCAAACACCCTC | |
| | | | CGTAGAGCCCGGGCCCTGCCCGCGGCTCCGGAGC | |
| | | | CAGCTCCATGGCGTGGACCTGGTGGTGGCAGAGGA | |
| | | | GGCGCCGAAGGTGGCCGTGGAGAAGGAGAAGGTG | |
| | | | GAGAAGACTACGCACAAGAAGACCTAGACGCCTTGT | |
| | | | TCGACGCCGTCGCAAGAGATACAGAGCCTCCAAGA | |
| | | | GCAAGAAAGAGACTACAGTTCGCAGGAGGAGAAAG | |
| | | | AACAGTCCTCCTCAGAAGAAGAGACGGACCCGAAG | |
| | | | AAAAAAGAGCAAAAACAGCAGCAGCGACTCCACCTC | |
| | | | CAGTTCCAAGAGCAGCAGCGACTCGGAAACCAACT | |
| | | | CCGACTCATCTTCCGAGAGCTACAGAAAACCCAAGC | |
| | | | GGGTCTCCACTTAAATCCTATGTTATCAAACCGGCT | |
| | | | GTAAATAAAGTTTACCTTTTTCCTCCCGAGGGGCCTA | |
| | | | AACCCATCTCTGGCTACAGAGCATGGGAAGACGAAT | |
| | | | TTACCACCTGTAAGTACTGGGACAGGCCTAGTAGAA | |
| | | | TTAACCACACAGACCCCCCCTTTTACCCCTGGATGC | |
| | | | CTAAATACAATGTAACCTTCAAACTTGGCTGGAAATA | |
| | | | A | |
| AB060596.1 | BAB69913.1 | ORF4 | ATGAGCTGGTGTACTCCAGTTGAAAATGCCTATAAG | 676 |
| | | | AGAGAGATCCACTTTCTCAGGGGCTGTCAACTGCTT | |
| | | | CACACTAGCTTTTGTGGTTGCGATGATTTTATTAATC | |
| | | | ATATTATTCGCCTACAAAATCTTCACGGCAACCTACA | |
| | | | CCAGCCCACGGGACCGTCCACACCTCCAGTGACCC | |
| | | | GTAGAGCTCTGGCCTTGCCGGCTGCTCCGGAGTCA | |
| | | | TGGCGTTCCGGTGGTGGTGGTGGAGACGCCGCCC | |
| | | | GCAGCGACGATGGACCCGGCGCCGATGGAGGAGA | |
| | | | CTACGAACCCGCCGACCTAGACGCACTGTACGACG | |
| | | | CCGTCGCCGCAGACCAAGAACACACAGTCAGAGCC | |
| | | | AGAAAAAGACTTCGGTTTCACACCGGAGAGCCAAGA | |
| | | | GTTACAGCAAGAAGACTTACGAGCACCCCAAGAAGA | |
| | | | AAGCCAAGAGGTACAGCAGCAGCGACTGCTCCAGC | |
| | | | TCAGACTCTCACAGCAGTTCAGACTCAGACAGCAGC | |
| | | | TCCAGCACCTGTTCGTACAAGTCCTCAAAACCCAAG | |
| | | | CAGGTCTCCACATAAACCCATTATTTTTAAACCATGC | |
| | | | ATAAATCAGGTCTTTATGTTTCCACCAGACACCCCCA | |
| | | | GACCTATTATAACTAAAGAAGGCTGGGAGGATGAGT | |
| | | | TTGTCACCTGCAAACACTGGGATAGGCCAGCTAGAT | |
| | | | CATACTACACAGACACACCTACTTACCCTTGGATGC | |
| | | | CCAAGGCACCCCCTCAATGCAATGTAAGCTTTAAAC | |
| | | | TTGGCTTTAAATAA | |
| AB060592.1 | BAB69897.1 | ORF4 | ATGAGCTTTGTAGAACCGTTACTAAGCAGCACCCAC | 677 |
| | | | CGAGAGATAGCATTCTACCATGGCTGTGTTCAAATG | |
| | | | CACAAGGCCTTCTGTGGCTGTGACAACTTTCTTACC | |
| | | | CACCTGCAGCGCATAACAACATACATCTCTGCTAAT | |
| | | | CAACACACTCCACCCAGCACACCCTCAAACACCCTC | |
| | | | CGTAGAGCCCGGGCCCTGCCCGCGGCTCCGGAGC | |
| | | | CAGCTCCATGGCGTGGACCTGGTGGTGGCAGAGGA | |
| | | | GGCGCCGAAGGTGGCCGTGGAGAAGGAGAAGGTG | |
| | | | GAGAAGACTACGCACCAGAAGACCTAGACGACTTGT | |
| | | | TCGCCGCCGTCGCAAGAGATACAGAGCCTCCAAGA | |
| | | | GCAAGAAAGAGACTACAGTTCGCAGGAGGAAAAAG | |
| | | | ACCAGTCCTCCTCAGAAGAAGAGAAGGACCCGAAG | |
| | | | AAAAAAGAGCAAAAACAGCAGCAGCGACTCCACCTC | |
| | | | CAGTTCCAAGAGCAGCAGCGACTCGGAAACCAACT | |
| | | | CCGACTCATCTTCCGAGAGCTACAGAAAACCCAAGC | |
| | | | GGGTCTCCACATAAATCCTATGTTATCAAACCGGCT | |
| | | | ATAAATAAAGTTTACCTTTTTCCTCCCGAGGGGCCTA | |
| | | | AACCCATCTCTGGCTACAGAGCATGGGAAGATGAGT | |
| | | | TCACCTGCTGTAAGTACTGGGACAGGCCTAGTAGAA | |
| | | | TTAACCACACAGACCCCCCCTTCTACCCCTGGATGC | |
| | | | CTAAGTACAATGTAACCTTTAAACTTGGCTGGAAATA | |
| | | | A | |
| AB060593.1 | BAB69901.1 | ORF4 | ATGAGTCTGTGGCGACCCCCGGTCCACAATGCCCC | 678 |
| | | | CGGCAGAGAGAGACTTTGGTTTCAGGCCTGTTACGA | |
| | | | ATCTCACAGTGCTTTTTGTGGCTGTGGTAGCTTTATT | |
| | | | CTTCATCTTACTAGCTTGGCTGCACGTTTTAATTTTC | |
| | | | AGGCCGGGCCACCGCCTCCCGGGGGTCCCCGGGC | |
| | | | GGAGACCCCGCCGATTCTGAGGGCGCTGCCGGCAC | |
| | | | CCCAGCCGCGCCGCCACCGCCAGACGGAGAACCC | |
| | | | CGGGTCTGAGCCATGGCCTGGAGATGGTGGTGGAG | |
| | | | ACGGCGCTGGAAGCCAAGAAGGCGGCCAGCGTGG | |
| | | | ACCAAGTACCGCAGACGCAGGTGGAGACGACTTCG | |
| | | | ACCCCGCAGACCTAGAAGACTTGCTCGCGGCCGTC | |
| | | | GAAGAAGACGAACATCGAGCTCCGAGAAAGAGCAG | |
| | | | AAGCCGAAGAAGACTCAGGTTCGGAAAAAGCGTCG | |
| | | | TTCACCTCGTCGCAAGAGAGAGAAGCCGAAGCCCA | |
| | | | AGAAAAGTTACCGATACAGCTCCAGCTCAGACAGCA | |
| | | | GCTCAGACAACAACAGCAGCTCCGAGTCCACTTGCA | |
| | | | GCAAGTCTTCCTCCAACTCCAAAAAACGAAGGCACA | |
| | | | TTTACATATAAACCCACTATTTTTGGCCCAAGGGAAC | |
| | | | ATGTAAACATGTTCGGTGAGTACCCAGATAGGAAGC | |
| | | | CCACTAAGGAAGATTGGCAGACCGAGTATGAGACCT | |
| | | | GCAGAGCCTTTGATAGACCCCCTAGAACCTTACTCA | |
| | | | CAGATCCCCCTTTCTACCCCTGGATGCCTAAACAAC | |
| | | | CCCCCACCTATCGTGTATCCTTCAAACTTGGCTTTCA | |
| | | | ATAA | |
| AB060595.1 | BAB69909.1 | ORF4 | ATGAATCTCTGGCGACCCCCTCTGAGAAATATCCCC | 679 |
| | | | CACAGGGAGAGATGTTGGCTTGAGGCCTGTCTCAG | |
| | | | AGCCCACGATTCTTTTTGTGGCTGTCCTAGTCCTATT | |
| | | | GTTCATTTTTCTAGTCTGGTTGCACGTTTTAATCTAC | |
| | | | AAGGAGGCCCGCCGCCAGAGGATGACTCCCCACAG | |
| | | | GGCGCGCCAGTCCTGAGGGCCCTGCCGGCACCGA | |
| | | | GCCCCCACAGGCACACCCGCACGGAGAACCCCTCC | |
| | | | GGTGAGCCATGGCCTACTCCTACTGGTGGCGCCGC | |
| | | | CGGAGGTGGCCGTGGAGAGGCCGATGGAGGCGCT | |
| | | | GGAGGCGCCGCAGACGAATACCGCGCCGAAGACCT | |
| | | | AGACGACCTGTTCGCCGCTATCGAAGGAGACCAAG | |
| | | | CAGCTCCAAGAAGACCCGCAAGAGCAAGAAAGAGA | |
| | | | CTCCTCTTCTTCGGAAGAAAGTCTCCCTACATCGTC | |
| | | | AGAAGAGACACCGCCAGCCCACCTACTCAGAGTAC | |
| | | | ACCTCAGAAAGCAGCTCCGGCAACAGCGAGACCTC | |
| | | | CGAGTCCAGCTCAGAGCCCTGTTCGCCCAAGTCCT | |
| | | | CAAAACGCAAGCGGGCCTACACATAAACCCCCTCTT | |
| | | | ATTGGCCCCGCAGTAAACAAGGTCTACTTGTTCCCT | |
| | | | GACAGGGCCCCTAAACCTCCACCTAGCTCGGGAGA | |
| | | | CTGGGCCACGGAGTACGCGGCGGCCGCCGCCTTC | |
| | | | GATAGACCCCCCAGAGGCAACCTGTCAGACAACCC | |
| | | | CTTCTATCCCTGGATGCCAACAAACACCAAATTCTCT | |
| | | | GTAACCTTTAAACTGGGGTGGAAACCCTGA | |
| AB064596.1 | BAB79311.1 | ORF4 | ATGCCGTGGAGACCGCCGGCTCATAACGTCCAGGG | 680 |
| | | | GCGAGAGAGCCAGTGGTTCGCGGCTTGTTTTCACG | |
| | | | GCCACGCTTCGTTTTGCGGCTGCGGTGACTTTATTG | |
| | | | GGCATATTAACAGCCTTGCTCCTCGCTTTCCTAACAA | |
| | | | CCAAGGACCCCCGCATCCACCTGCCTTAAACAGGC | |
| | | | CACCTGCACAGGGCCCAGAAAGCCCCGGGGGTTCC | |
| | | | ATACTACCCCTGCCAGCCCTACCGGCACCACCTGAT | |
| | | | CCGCCACCACGGCCTGGTGGTGGGGAAGACGGTG | |
| | | | GCGACGCCGCCCGTGGGGCCGCTGGCGCCGCCGA | |
| | | | AGGCGCGTATGGAGAAGAAGACCTAGAACTGCTGTT | |
| | | | CGCCGCCGCCGAGGAAGACGATATGTCGCCCAAAG | |
| | | | CGCCCAAAGCTCGACACACGTCCCGAAGGACTACC | |
| | | | AGAGGAGCAAAGAGGAGCTTACAATTTACTCCAAGC | |
| | | | CCTCGAAGACTCAGCCCAGTCGGAAGAAAGCGACC | |
| | | | AAGAAGAAATGCCTCCCCTCGAAGAAGAACAAGTAC | |
| | | | TCCACGAGCAAAAGAAAGAGGCGCTCCTCCAGCAG | |
| | | | CTCCAGCAGCAGAAACACCACCAGCGAGTCCTCAA | |
| | | | GCGAGGCCTCAGACTCCTCCTCGGAGACGTCCTGA | |
| | | | AACTCCGCCGGGGTCTACACATAGACCCGGTCCTTA | |
| | | | CATAGCACCCCCTCCATACATCCCTGACCTTCTTTTT | |
| | | | CCCAACACCCAAAAAAAAAAAAAATTTTCCAACTTCG | |
| | | | ATTGGGCTACAGAATACCAGCTTGCTACCGCTTTCG | |
| | | | ACCGCCCTCTCCGCCACTACCCCTTAGACCTCCCGC | |
| | | | ACTACCCGTGGCTACCAAAAAAGCCCAATACCCACT | |
| | | | CTACCTATAGAGTGTCCTTTCAACTAAAAGCCCCCC | |
| | | | AATAA | |
| AB064597.1 | BAB79315.1 | ORF4 | ATGCCGTGGAGACCGCCGGTGCATAGTGTCCAGGG | 681 |
| | | | GCGAGAGGATCAGTGGTTCGCGAGCTTTTTTCACGG | |
| | | | CCACGCTTCATTTTGCGGTTGCGGTGACGCTGTTGG | |
| | | | CCATCTTAATAGCATTGCTCCTCGCTTTCCTCGCGC | |
| | | | CGGTCCACCAAGGCCCCCTCCGGGGCTAGAGCAGC | |
| | | | CTAACCCCCCGCAGCAGGGCCCGGCCGGGCCCGG | |
| | | | AGGGCCGCCCGCCATCTTGGCGCTGCCGGCTCCGC | |
| | | | CCGCGGAGCCTGACGACCCGCAGCCACGGCGTGG | |
| | | | TGGTGGGGACGGTGGCGCCGCCGCTGGCGCCGCA | |
| | | | GGCGACCGTGGAGACCGAGACTACGACGAAGAAGA | |
| | | | GCTAGACGAGCTTTTCCGCGCCGCCGCCGAAGACG | |
| | | | ATTTGTCTCCCATCAAAGCGAAACAAGCTCGACTCG | |
| | | | GCCTTCAGAGGAGAAAACCCAGAGCAAAAAGAATGC | |
| | | | TATTCTCTCCTCAAAGCACTCGAGGAAGAAGAGACC | |
| | | | CCAGAAGAAGAAGAACCAGCACCCCAAGAAAAAGC | |
| | | | CCAGAAAGAGGAGCTACTCCACCAGCTCCAGCTCC | |
| | | | AGAGACGCCACCAGCGAGTCCTCAGACGAGGGCTC | |
| | | | AAGCTCGTCTTTACAGACATCCTCCGACTCCGCCAG | |
| | | | GGAGTCCACTGGAACCCCGAGCTCACATAGAGCCC | |
| | | | CCACCTTACATACCAGACCTACTTTTTCCCAATACTG | |
| | | | GTAAAAAAAAAAAATTCTCTCCCTTCGACTGGGAAAC | |
| | | | GGAGGCCCAGCTAGCAGGGATATTCAAGCGTCCTA | |
| | | | TGCGCTTCTATCCCTCAGACACCCCTCACTACCCGT | |
| | | | GGTTACCCCCCAAGCGCGATATCCCGAAAATATGTA | |
| | | | ACATAAACTTCAAAATAAAGCTGCAAGAGTGA | |
| AB064599.1 | BAB79323.1 | ORF4 | ATGCCGTGGTCTCTGCCGAGACATAATATCAGAACG | 682 |
| | | | AGAGAAGATCTCTGGGTGCAATCGATTCTTTATTCAC | |
| | | | ATGACACTTTTTGTGGCTGTGATAATATTCCTGAGCA | |
| | | | TCTTACTGGCCTCCTGGGCGGCGTACGACCAGCTC | |
| | | | CACCTAGAAACCCAGGACCCCCTACCATACGGAGC | |
| | | | CTGCCGGCACTGCCGCCAGCTCCGGAACCCCCTGA | |
| | | | GGAACCACGGCGTGGTGGAGATACAGACGGAGACC | |
| | | | GTGGAGAAGATGGAGGAGACGCCGCTGGGGCCTAC | |
| | | | GAACCCGAAGACCTAGAAGAACTTTTCGCCGCCGC | |
| | | | CGAGCAAGACGATATGCGTCGTGTCCAAAAAACCCC | |
| | | | GATTCGACACTCCCCACCACGGGCAGCTATCAAACC | |
| | | | AAGAAGAAGACGCCTTGTCTATCCTCAGACAACCCC | |
| | | | AAAAAGAGCAAGAAGAGACCACCTCCGAGGAAGAA | |
| | | | CAAGCACTCCAAAAAGAAGAGGAGCAAAAAGAAAAG | |
| | | | CTCCTACAGCAACTCAGAGTCCAGCGACAGCACCA | |
| | | | GCGAGTCCTCAGACAGGGAATCAAACACCTCATGG | |
| | | | GAGACGTCCTCCGACTCAGACAGGGAGTCCACTGG | |
| | | | AACCCAGTCCTATAATACTTCCACCAGAACCAATACC | |
| | | | AGACCTCTTATTCCCCAATACTGGTAAAAAAAAAAAA | |
| | | | TTCTCTCTCTTCGACTGGGAGTGCGAGAGGGATCTA | |
| | | | GCATGTGCATTCTGCCGTCCCATGCGCTTCTATCCC | |
| | | | TCAGACAACCCAACTTACCCGTGGTTACCCCCCAAG | |
| | | | CGAGATATCCCCAAAATATGTAAAGTAAACTTCAAAA | |
| | | | TAAATTTCACTGAATGA | |
| AB064600.1 | BAB79327.1 | ORF4 | ATGTCGTGGAGACCGCCGAGCCAAAATTTACTGCAA | 683 |
| | | | AGAGAAGAGGCCTGGTACTCAGCTTTTCTTAGCTCG | |
| | | | CATTCTACATTTTGCGGTTGTACTGACCCTCTGCTGC | |
| | | | ATATTACTCTCATTGCTGGCCGCCTTACTAACCCCGT | |
| | | | ACCCGTCACCCGCCAACCGGAGACCCCTCCTAACG | |
| | | | GCCTCAGGGGGCTGCCGGCACTGCCAGCACCCCCT | |
| | | | GAACCACCAGCACCGCCACCACGGCCTGGGGATGG | |
| | | | TACCGGAGAAGAAGATGGCGCCCATGGAGAAGGAG | |
| | | | AAGGTGGGCGATACGCAGAAGAAGACCTAGAAGAA | |
| | | | CTGTTCGCCGCCGCGGCAGAAGACGATATGCGTCG | |
| | | | CATCCAAAAGACCCCGATTCGACACTCCAGTCCAAG | |
| | | | GGCAGCTCGAAAGCCAAGAAGAAGAAAGCTATCGTT | |
| | | | TACTCAGAGCACTCCAAAAAGAGCAAGAGACAAGCA | |
| | | | GCTCGGAAGAGGAGCAGCCACAAAACCAAGAGATC | |
| | | | CAAGAAAAACTACTCCTCCAGCTCCAGCAGCAGCGA | |
| | | | CAACAGCAGCGACTCCTCGCAAAGGGAATCAAGCA | |
| | | | CCTCCTCGGAGATGTCCTCCGACTCCGAAAAGGAGT | |
| | | | CCACTGGGACCCGGTCCTTACATAGCACCTCCAGAA | |
| | | | CCTATCCCAGACCTTTTGTTCCCCAGTACTAAAAAAA | |
| | | | AAAAGAAATTTTCAAAATTAGACTGGGAGAACGAGG | |
| | | | CTCAAATAGCAGGGTGGTTAGACAGGCCTATGAGG | |
| | | | CTGTATCCTGGGGACCCCCCCTTCTACCCTTGGCTA | |
| | | | CCCCGAAAGCCACCTACCCAGCCTACATGTAGGGTA | |
| | | | AGCTTCAAAATAAAGCTAGATGATTAA | |
| AB064601.1 | BAB79331.1 | ORF4 | ATGTCGTGGGCTCCGCCGCTATTCAACTCGAAACAG | 684 |
| | | | AGAGAGGACCAGTGGTACCAGTCAATTATTTTCAGC | |
| | | | CATAATACTTTTTGCGGCTGCGGTGACCTTGTTAGG | |
| | | | CATTTTTGCGTCGTTGCTTCTCGCTTTACTGAGCCTC | |
| | | | CTGTAGTGCCGGCCCTACCGGCACCGGTACCGGCA | |
| | | | CCGCCACGGCGTGGTACAGAAGAAGAAGGTGGAGA | |
| | | | CCGTGGAGAAGACGCCGCAGACCGTGGACCCTACG | |
| | | | CAGAAGAAGAGCTAGAAGATTTGTTCGCCGCCGCC | |
| | | | CGAGAAGACGATATGCGTCGTCTTCAAAAGACCCCG | |
| | | | ACTGGAAACCCAGTACAAAGGAACCCAAGAAACCCC | |
| | | | AGAAGAAGACGCCTACACTTTACTCAAAGCACTCCA | |
| | | | AAAAGAGCAAGAGAGCAGCAGCTCGGAAGAAGAAC | |
| | | | TCCCACAAGAAGAGCAAGAGATCCAAAAAACACAAC | |
| | | | TCCTCAAGCAGCTCCAACTCCAGCAGCAGCAACAGC | |
| | | | GAATCCTCAAGAGGGGAATCAGACACCTCTTCGGAG | |
| | | | ACGTCCTCCGACTCAGAAAAGGAGTCCACTCCAACC | |
| | | | CAGACCTATTATAATACCAGCAGAGGAAATCCCAGA | |
| | | | CCTGCTTTTCCCCAATACTGGTAAAAAAAAAAAATTC | |
| | | | TCTCCATTCGATTGGGAGACAGAGCAGCAGCTCGCA | |
| | | | TGCTGGATGCGGCGCCCCATGCGCTTCTATCCAACA | |
| | | | GACCCCCCGTTCTACCCCTGGCTACCCCCCAAGCG | |
| | | | AGATATCCCCAATATATGTAAAGTCAACTTCAAAATA | |
| | | | AATTACTCAGAGTAA | |
| AB064602.1 | BAB79335.1 | ORF4 | ATGCCGTGGCATCCACCGGGCTACAACGTTCAACA | 685 |
| | | | GAGAGAAGAGCTCTGGGTACAGACAGTTACTACTTC | |
| | | | ACATGCTACTTTTTGCGGCTGTGGTGACCCTAGTAG | |
| | | | CCATCTTCACCGCATTCTTAGCCGCCTTAATAACAG | |
| | | | CAGCCGGCGGCCCCCCGAAACCCCAAACCCCATTC | |
| | | | GTGCCCTACCGGCCCTACCGGCACCCCAAGAACCT | |
| | | | GAACAGCCGCCATCACGGCCTGGTACCGGTACAGA | |
| | | | AGAAGGCCATGGCGCCGAAGGAGGCGACCGAGGT | |
| | | | GGGGCCTACGCAGAAGAAGATTTAGAAGATCTTTTC | |
| | | | GCGGCCGCGGAAGAAGACGATATGCGTCGTGTCAA | |
| | | | AAAGACCCCGATTGGAAACCCAGTACCACGGCCAA | |
| | | | CACGAAAGCCAAGAAGAAGACGCCTATCTTTTACTC | |
| | | | AAACAACTCCAGGAAGAGCAAGAAACGAGCAGTTCG | |
| | | | GAGGGAGAACAAGCACCCCAAGAAAAAACACTCCAA | |
| | | | AAAGAAAAGCTCCTCAAGCAGCTGCAGCTCCACAAG | |
| | | | CAGCAGCAGCAACTCCTCAGAAAAGGAATCAGACAC | |
| | | | CTCCTCGGGGACGTCCTCCGACTCAGACGGGGAGT | |
| | | | CCACTGGGACCCAGGCCTATAGTACTGCCTCCAGA | |
| | | | GCCTATTCCAGACTTGCTTTTCCCAAATACTAAAAAA | |
| | | | AAAAAGAAATTTTCGCCCTTAGACTGGGAGAACGAG | |
| | | | GCTCAAATAGCAGGGTGGTTAGACAGGCCTATGAG | |
| | | | GCTGTATCCTGGGGACAACCCCTTCTACCCGTGGCT | |
| | | | ACCAAAAAAGCCACCTACCCACCCTACATGTAGAGT | |
| | | | AACCTTCAAAATAAAGCTAGATGATTAA | |
| AB064603.1 | BAB79339.1 | ORF4 | ATGTCGTGGCGACCGCCGTTGCATTCTATCCAAGGC | 686 |
| | | | AGAGAAGATCAATGGTATGCAGGCATCTTTCATACG | |
| | | | CATTTTGCTTTTTGCGGTTGTGGTGACCCTGTTGGG | |
| | | | CGTATTAACCGCATTGCTCACCGCTTTCCTAACGCC | |
| | | | GGTCCCCCGAGACCACCTCCAGGGCTAGACCAGCC | |
| | | | CAACCTCGGAGGGCCGGAAGGTCCAGGAGGTGCC | |
| | | | CCTAGAGCCCTGCCAGCCCTGCCGGCCCCGGCAGA | |
| | | | GCCAGAGCCGGCACCACGGCGTGGTGGTGGGGCC | |
| | | | GATGGAGACAGCGCCGCTGGGGCCGCCGCCGCCG | |
| | | | CAGACCATGGAGGGTACGACGAAGGAGACCTAGAA | |
| | | | GATCTTTTCGCCGCCGCCGCCGAGGACGATATGGC | |
| | | | CTTTATCAAGAAAAAGAAACAAGCTCGACACAAAGAT | |
| | | | GCCAGGCCCCCCAACCCCCGAAAAAGAAAGCTACA | |
| | | | CTTTACTCCAAGCCCTCCAAGAGTCGGGCCAGGAG | |
| | | | AGCAGCTCCCAGGACGAAGAACAAGCACCCCAAAA | |
| | | | AGAAGAGAACCAGAAAGAAGCGCTCGTGGAGCAGC | |
| | | | TCCAGCTCCAGAAACAGCACCAGCGAGTCCTCAAG | |
| | | | CGAGGCCTCAAACTCCTCTTGGGAGACGTCCTCCG | |
| | | | ACTCCGCCGCGGAGTCCACTGGGACCCCCTCCTAT | |
| | | | CCTAATTCAGGGTCCCTCTATCCCAGACCTGCTTTT | |
| | | | CCCTAACACTCAAAAAAAACCCAAATTTTCCAACTTC | |
| | | | GACTGGGCCACCGAGTACCAAATAGCCAAGTGGCC | |
| | | | AGACCGCCCTTTGAGGCACTACCCCTCAGACCTCCC | |
| | | | TCACTACCCGTGGCTACCAAAAAAGCCACCTACCCA | |
| | | | GCCTACATGTAGAGTAAGTTTCAAATTAAAGCTTGAT | |
| | | | GCCTAA | |
| AB064604.1 | BAB79343.1 | ORF4 | ATGAGTATTTGGAGGCCTCCACTGCACAATGTCCCG | 687 |
| | | | GGACTCGAACACCTCTGGTACGAGTCAGTGCATCGT | |
| | | | AGCCATGCTGCTGTTTGTGGCTGTGGGGATCCTGTA | |
| | | | CGCCATCTTACTGCTCTTGCTGAAAGATATGGCATT | |
| | | | CCGGGAGGGTCGCGGTCTTCTGGGGCACCGGGAG | |
| | | | TAGGGGGCAACCACAACCCTCCCCAGATCCGTCGA | |
| | | | GCCCGCCACCCGGCGGCTGCTCCGGACCCCCCAG | |
| | | | CAGGTAACCAGCCTCCGGCCCTGCCATGGCATGGG | |
| | | | GATGGTGGAAACGAAAGCGGCGCTGGTGGTGGAGA | |
| | | | AAGCGGTGGACCCGTGGCCGACTTCGCAGACGATG | |
| | | | GCCTAGACGATCTCGTCGCCGCCCTCGACGAAGAA | |
| | | | GAAAGAAGACTTCAATATCCAAGAGAGACAACAAAG | |
| | | | AGAACAGAGACCGTGGACGAGCGAAAGCGAGAGCG | |
| | | | AAGCAGAAGCCCAAGAAGAGACGCAGGCGGGCTCG | |
| | | | GTCCGAGAGCAGCTCCAGCAGCAGCTCCAAGAGCA | |
| | | | GTTTCAACTCCGAAGAGGGCTCAAGTGCCTCTTCGA | |
| | | | GCAGTTAGTCAGAACCCAACAGGGAGTCCACGTAG | |
| | | | ATCCCTGCCTCGTGTAGGCCCGGAGCAGTGGCTAC | |
| | | | TCCCCGAGAGAAAGCCTAAGCCCGCTCCTACTTCAG | |
| | | | GAGACTGGGCTATGGAGTACCTAATGTGCAAAATAA | |
| | | | TGAATAGGCCTCCTCGCTCTCAGCTTACTGACCCCC | |
| | | | CATTTTACCCTTACTGCAAAAATAATTACAATGTAAC | |
| | | | CTTTCAGCTTAACTACAAATAA | |
| AB064606.1 | BAB79351.1 | ORF4 | ATGAGCTTCTGGAGACCTCCGGTGCACAATGCCAC | 688 |
| | | | GGGGATCCAGCGCCTGTGGTACGAGTCCTTTCACC | |
| | | | GTGGCCATGCTGCTTTTTGTGGTTGTGGGGATCCTA | |
| | | | TACTTCACATTACTGCACTTGCTGAGACATATGGCCA | |
| | | | TCCAACAGGCCCGAGACCTTCTGGGCCACCGCGAG | |
| | | | TAGACCCCGATCCCCAGATCCGTAGAGCCAGGCCT | |
| | | | GCCCCGGCCGCTCCGGAGCCCTCACAGGTTGAGCC | |
| | | | GAGACCTGCCCTGCCATGGCATGGGGATGGTGGAA | |
| | | | GCGACGGCGGCGCTGGTGGTTCCGGAAGCGGTGG | |
| | | | ACCCGTGGCAGACTTCGCAGACGATGGCCTCGATC | |
| | | | AGCTCGTCGCCGCCCTAGACGACGAAGAAAAAAGG | |
| | | | CTCAGATTCACTCCAAAGAGAATCGAGACCGTGGAG | |
| | | | CAACTCGGAGACCGAGGCAGAGACAGAAGCCCCCT | |
| | | | CGGAAGAAGAGCCGGAGAACCAAGAAGAACAAGTA | |
| | | | CTCCAGTTGCAGCTCCGACAGCAGCTCCGAGAACA | |
| | | | GCGAAAACTCAGACAGGGAATCCAGTGCCTCTTCGA | |
| | | | GCAACTGATAACAACCCAACAGGGGGTTCACAAAAA | |
| | | | CCCATTGCTAGAGTAGGCCCAGAGCAGTGGCTGTTT | |
| | | | CCCGAGAGAAAGCCAAAACCACCTCCCACCGCCCA | |
| | | | GGACTGGGCGGAGGAGTACACTGCCTGTAAATACT | |
| | | | GGGGTAGGCCACCTCGCAAATTCCTCACAGACACG | |
| | | | CCATTCTATACTCACTGCAAGACCAATTACAATGTAA | |
| | | | CCTTTATGCTTAACTATCAATAA | |
| FJ426280.1 | ACK44074.1 | ORF4 | ATGGGACTGGCGACGGGGGCTTTTTGGTGCAGATG | 689 |
| | | | CTATCCAGAGAGTGTCACAAAAACCGGAAGATGCTC | |
| | | | TCCGCTTTACAAACCCTTTCAAGAGACCCAGATATCT | |
| | | | TCCCCCGACAGACGGAGAAGACTACCGACAAGAAG | |
| | | | AAGACTTCGCTTTACAGGAAAGAAGACGGCGCACAT | |
| | | | CCACAGAAGAAGTCCAGGACGAGGAGAGCCCCCCG | |
| | | | CAAAACGCGCCGCTCCTACAGCAGCAGCAGCAGCA | |
| | | | GCGGGAGCTCTCAGTCCAGCACGCGGAGCAGCAGC | |
| | | | GACTCGGAGTCCAACTCCGATACATCCTCCAAGAAG | |
| | | | TCCTCAAAACGCAAGCGGGTCTCCACCTAAACCCCC | |
| | | | TATTATTAGGCCCGCCACAAACAAGGTGTATATCTTT | |
| | | | GAGCCCCCCAGAGGCCTACTCCCCATAGTGGGAAA | |
| | | | AGAAGCCTGGGAGGACGAGTACTGCACCTGCAAGT | |
| | | | ACTGGGATCGCCCTCCCAGAACCAACCACCTAGACA | |
| | | | CCCCCACTTATCCCTAG | |

In some embodiments, the genetic element may comprise one or more sequences or a fragment of a sequence from a substantially non-pathogenic virus having at least about 60%, 70% 80%, 85%, 90% 95%, 96%, 97%, 98% and 99% nucleotide sequence identity to any one of the nucleotide sequences described herein, e.g., Table 20.

TABLE 20
Examples of Anelloviruses and their sequences. Accessions numbers and related sequence
information may be obtained at www.ncbi.nlm.nih.gov/genbank/, as referenced on Jun. 12, 2017.

| Accession # | Description |
|---|---|
| AB026345.1 | TT virus genes for ORF1 and ORF2, complete cds, isolate: TRM1 |
| AB026346.1 | TT virus genes for ORF1 and ORF2, complete cds, isolate: TK16 |
| AB026347.1 | TT virus genes for ORF1 and ORF2, complete cds, isolate: TP1-3 |
| AB030487.1 | TT virus gene for pORF2a, pORF2b, pORF1, complete cds, clone: JaCHCTC19 |
| AB030488.1 | TT virus gene for pORF2a, pORF2b, pORF1, complete cds, clone: JaBD89 |
| AB030489.1 | TT virus gene for pORF2a, pORF2b, pORF1, complete cds, clone: JaBD98 |
| AB038340.1 | TT virus genes for ORF2s, ORF1, ORF3, complete cds |
| AB038622.1 | TT virus genes for ORF2, ORF1, ORF3, complete cds, isolate: TTVyon-LC011 |
| AB038623.1 | TT virus genes for ORF2, ORF1, ORF3, complete cds, isolate: TTVyon-KC186 |
| AB038624.1 | TT virus genes for ORF2, ORF1, ORF3, complete cds, isolate: TTVyon-KC197 |
| AB041821.1 | TT virus mRNA for VP1, complete cds |
| AB050448.1 | Torque teno virus genes for ORF1, ORF2, ORF3, ORF4, complete cds, isolate: TYM9 |
| AB060592.1 | Torque teno virus gene for ORF1, ORF2, ORF3, ORF4, clone: SAa-39 |
| AB060593.1 | Torque teno virus gene for ORF1, ORF2, ORF3, ORF4, complete cds, clone: SAa-38 |
| AB060595.1 | TT virus gene for ORF1, ORF2, ORF3, ORF4, complete cds, clone: SAj-30 |
| AB060596.1 | TT virus gene for ORF1, ORF2, ORF3, ORF4, complete cds, clone: SAf-09 |
| AB064596.1 | Torque teno virus DNA, complete genome, isolate: CT25F |
| AB064597.1 | Torque teno virus DNA, complete genome, isolate: CT30F |
| AB064599.1 | Torque teno virus DNA, complete genome, isolate: JT03F |
| AB064600.1 | Torque teno virus DNA, complete genome, isolate: JT05F |
| AB064601.1 | Torque teno virus DNA, complete genome, isolate: JT14F |
| AB064602.1 | Torque teno virus DNA, complete genome, isolate: JT19F |
| AB064603.1 | Torque teno virus DNA, complete genome, isolate: JT41F |
| AB064604.1 | Torque teno virus DNA, complete genome, isolate: CT39F |
| AB064606.1 | Torque teno virus DNA, complete genome, isolate: JT33F |
| AF079173.1 | TT virus strain TTVCHN1, complete genome |
| AF116842.1 | TT virus strain BDH1, complete genome |
| AF122917.1 | TT virus isolate JA4, complete genome |
| AF122919.1 | TT virus isolate JA10 unknown genes |
| AF129887.1 | TT virus TTVCHN2, complete genome |
| AF254410.1 | TT virus ORF2 protein and ORF1 protein genes, complete cds |
| AF298585.1 | TT virus Polish isolate P/1C1, complete genome |
| AF315076.1 | TTV-like virus DXL1 unknown genes |
| AF315077.1 | TTV-like virus DXL2 unknown genes |
| AF345521.1 | TT virus isolate TCHN-G1 Orf2 and Orf1 genes, complete cds |
| AF345522.1 | TT virus isolate TCHN-E Orf2 and Orf1 genes, complete cds |
| AF345525.1 | TT virus isolate TCHN-D2 Orf2 and Orf1 genes, complete cds |
| AF345527.1 | TT virus isolate TCHN-O2 Orf2 and Orf1 genes, complete cds |
| AF345528.1 | TT virus isolate TCHN-F Orf2 and Orf1 genes, complete cds |
| AF345529.1 | TT virus isolate TCHN-G2 Orf2 and Orf1 genes, complete cds |
| AF371370.1 | TT virus ORF1, ORF3, and ORF2 genes, complete cds |
| AJ620212.1 | Torque teno virus, isolate tth6, complete genome |
| AJ620213.1 | Torque teno virus, isolate tth10, complete genome |
| AJ620214.1 | Torque teno virus, isolate tth11g2, complete genome |
| AJ620215.1 | Torque teno virus, isolate tth18, complete genome |
| AJ620216.1 | Torque teno virus, isolate tth20, complete genome |
| AJ620217.1 | Torque teno virus, isolate tth21, complete genome |
| AJ620218.1 | Torque teno virus, isolate tth3, complete genome |
| AJ620219.1 | Torque teno virus, isolate tth9, complete genome |
| AJ620220.1 | Torque teno virus, isolate tth16, complete genome |
| AJ620221.1 | Torque teno virus, isolate tth17, complete genome |
| AJ620222.1 | Torque teno virus, isolate tth25, complete genome |
| AJ620223.1 | Torque teno virus, isolate tth26, complete genome |
| AJ620224.1 | Torque teno virus, isolate tth27, complete genome |
| AJ620225.1 | Torque teno virus, isolate tth31, complete genome |
| AJ620226.1 | Torque teno virus, isolate tth4, complete genome |
| AJ620227.1 | Torque teno virus, isolate tth5, complete genome |
| AJ620228.1 | Torque teno virus, isolate tth14, complete genome |
| AJ620229.1 | Torque teno virus, isolate tth29, complete genome |
| AJ620230.1 | Torque teno virus, isolate tth7, complete genome |
| AJ620231.1 | Torque teno virus, isolate tth8, complete genome |
| AJ620232.1 | Torque teno virus, isolate tth13, complete genome |
| AJ620233.1 | Torque teno virus, isolate tth19, complete genome |
| AJ620234.1 | Torque teno virus, isolate tth22g4, complete genome |
| AJ620235.1 | Torque teno virus, isolate tth23, complete genome |
| AM711976.1 | TT virus sle1957 complete genome |
| AM712003.1 | TT virus sle1931 complete genome |
| AM712004.1 | TT virus sle1932 complete genome |
| AM712030.1 | TT virus sle2057 complete genome |
| AM712031.1 | TT virus sle2058 complete genome |
| AM712032.1 | TT virus sle2072 complete genome |
| AM712033.1 | TT virus sle2061 complete genome |
| AM712034.1 | TT virus sle2065 complete genome |
| AY026465.1 | TT virus isolate L01 ORF2 and ORF1 genes, complete cds |
| AY026466.1 | TT virus isolate L02 ORF2 and ORF1 genes, complete cds |
| DQ003341.1 | Torque teno virus clone P2-9-02 ORF2 (ORF2), ORF1A (ORF1A), and ORF1B (ORF1B) |
| | genes, complete cds |
| DQ003342.1 | Torque teno virus clone P2-9-07 ORF2 (ORF2), ORF1A (ORF1A), and ORF1B (ORF1B) |
| | genes, complete cds |
| DQ003343.1 | Torque teno virus clone P2-9-08 ORF2 (ORF2), ORF1A (ORF1A), and ORF1B (ORF1B) |
| | genes, complete cds |
| DQ003344.1 | Torque teno virus clone P2-9-16 ORF2 (ORF2), ORF1A (ORF1A), and ORF1B (ORF1B) |
| | genes, complete cds |
| DQ186994.1 | Torque teno virus clone P601 ORF2 (ORF2) and ORF1 (ORF1) genes, complete cds |
| DQ186995.1 | Torque teno virus clone P605 ORF2 (ORF2) and ORF1 (ORF1) genes, complete cds |
| DQ186996.1 | Torque teno virus clone BM1A-02 ORF2 (ORF2) and ORF1 (ORF1) genes, complete cds |
| DQ186997.1 | Torque teno virus clone BM1A-09 ORF2 (ORF2) and ORF1 (ORF1) genes, complete cds |
| DQ186998.1 | Torque teno virus clone BM1A-13 ORF2 (ORF2) and ORF1 (ORF1) genes, complete cds |
| DQ186999.1 | Torque teno virus clone BM1B-05 ORF2 (ORF2) and ORF1 (ORF1) genes, complete cds |
| DQ187000.1 | Torque teno virus clone BM1B-07 ORF2 (ORF2) and ORF1 (ORF1) genes, complete cds |
| DQ187001.1 | Torque teno virus clone BM1B-11 ORF2 (ORF2) and ORF1 (ORF1) genes, complete cds |
| DQ187002.1 | Torque teno virus clone BM1B-14 ORF2 (ORF2) and ORF1 (ORF1) genes, complete cds |
| DQ187003.1 | Torque teno virus clone BM1B-08 ORF2 (ORF2) gene, complete cds; and nonfunctional |
| | ORF1 (ORF1) gene, complete sequence |
| DQ187004.1 | Torque teno virus clone BM1C-16 ORF2 (ORF2) and ORF1 (ORF1) genes, complete cds |
| DQ187005.1 | Torque teno virus clone BM1C-10 ORF2 (ORF2) and ORF1 (ORF1) genes, complete cds |
| DQ187007.1 | Torque teno virus clone BM2C-25 ORF2 (ORF2) gene, complete cds; and nonfunctional |
| | ORF1 (ORF1) gene, complete sequence |
| D0361268.1 | Torque teno virus isolate ViPi04 ORF1 gene, complete cds |
| EF538879.1 | Torque teno virus isolate CSC5 ORF2 and ORF1 genes, complete cds |
| EU305675.1 | Torque teno virus isolate LTT7 ORF1 gene, complete cds |
| EU305676.1 | Torque teno virus isolate LTT10 ORF1 gene, complete cds |
| EU889253.1 | Torque teno virus isolate ViPi08 nonfunctional ORF1 gene, complete sequence |
| FJ392105.1 | Torque teno virus isolate TW53A25 ORF2 gene, partial cds; and ORF1 gene, complete cds |
| FJ392107.1 | Torque teno virus isolate TW53A27 ORF2 gene, partial cds; and ORF1 gene, complete cds |
| FJ392108.1 | Torque teno virus isolate TW53A29 ORF2 gene, partial cds; and ORF1 gene, complete cds |
| FJ392111.1 | Torque teno virus isolate TW53A35 ORF2 gene, partial cds; and ORF1 gene, complete cds |
| FJ392112.1 | Torque teno virus isolate TW53A39 ORF2 gene, partial cds; and ORF1 gene, complete cds |
| FJ392113.1 | Torque teno virus isolate TW53A26 ORF2 gene, complete cds; and nonfunctional ORF1 |
| | gene, complete sequence |
| FJ392114.1 | Torque teno virus isolate TW53A30 ORF2 and ORF1 genes, complete cds |
| FJ392115.1 | Torque teno virus isolate TW53A31 ORF2 and ORF1 genes, complete cds |
| FJ392117.1 | Torque teno virus isolate TW53A37 ORF1 gene, complete cds |
| FJ426280.1 | Torque teno virus strain SIA109, complete genome |
| GU797360.1 | Torque teno virus clone 8-17, complete genome |
| HC742700.1 | Sequence 7 from Patent WO2010044889 |
| HC742710.1 | Sequence 17 from Patent WO2010044889 |

In some embodiments, the genetic element comprises one or more sequences with homology or identity to one or more sequences from one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus. Since, in some embodiments, recombinant retroviruses are defective, assistance may be provided order to produce infectious particles. Such assistance can be provided, e.g., by using helper cell lines that contain plasmids encoding all of the structural genes of the retrovirus under the control of regulatory sequences within the LTR. Suitable cell lines for replicating the curons described herein include cell lines known in the art, e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is recognized in the art as a substitution of one amino acid for another amino acid that has similar properties.

Identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more.

In some embodiments, the genetic element comprises a nucleotide sequence with at least about 75% nucleotide sequence identity, at least about 80%, 85%, 90% 95%, 96%, 97%, 98%, 99% or 100% nucleotide sequence identity to any one of the nucleotide sequences described herein, e.g., Table 19 or Table 20. Since the genetic code is degenerate, a homologous nucleotide sequence can include any number of “silent” base changes, i.e. nucleotide substitutions that nonetheless encode the same amino acid.

Gene Editing Component

The genetic element of the synthetic curon may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the curon includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes may be included in the curon. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the curon includes a gene encoding a Cas protein, e.g., a Cas9 protein, may be from any of a variety of prokaryotic species. In some embodiments, the curon includes a gene encoding a particular Cas protein, e.g., a particular Cas9 protein, is selected to recognize a particular protospacer-adjacent motif (PAM) sequence. In some embodiments, the curon includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the curon includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas9 generates only a single-strand break; a catalytically inactive Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, Mass. 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the curon comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

As used herein, a “biologically active portion of an effector domain” is a portion that maintains the function (e.g. completely, partially, or minimally) of an effector domain (e.g., a “minimal” or “core” domain). In some embodiments, the curon includes a gene encoding a fusion of a dCas9 with all or a portion of one or more effector domains to create a chimeric protein useful in the methods described herein. Accordingly, in some embodiments, the curon includes a gene encoding a dCas9-methylase fusion. In other some embodiments, the curon includes a gene encoding a dCas9-enzyme fusion with a site-specific gRNA to target an endogenous gene.

In other aspects, the curon includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Proteinaceous Exterior

In some embodiments, the curon, e.g., synthetic curon, comprises a proteinaceous exterior that encloses the genetic element. The proteinaceous exterior can comprise a substantially non-pathogenic exterior protein that fails to elicit an immune response in a mammal. In some embodiments, the synthetic curon lacks lipids in the proteinaceous exterior. In some embodiments, the synthetic curon lacks a lipid bilayer, e.g., a viral envelope. In some embodiments, the interior of the synthetic curon is entirely covered (e.g., 100% coverage) by a proteinaceous exterior. In some embodiments, the interior of the synthetic curon is less than 100% covered by the proteinaceous exterior, e.g., 95%, 90%, 85%, 80%, 70%, 60%, 50% or less coverage. In some embodiments, the proteinaceous exterior comprises gaps or discontinuities, e.g., permitting permeability to water, ions, peptides, or small molecules, so long as the genetic element is retained in the curon.

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or non-pathogenic in a host.

### Vectors

The genetic element described herein may be included in a vector. Suitable vectors as well as methods for their manufacture and their use are well known in the prior art.

In one aspect, the invention includes a vector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid.

The genetic element or any of the sequences within the genetic element can be obtained using any suitable method. Various recombinant methods are known in the art, such as, for example screening libraries from cells harboring viral sequences, deriving the sequences from a vector known to include the same, or isolating directly from cells and tissues containing the same, using standard techniques. Alternatively or in combination, part or all of the genetic element can be produced synthetically, rather than cloned.

In some embodiments, the vector includes regulatory elements, nucleic acid sequences homologous to target genes, and various reporter constructs for causing the expression of reporter molecules within a viable cell and/or when an intracellular molecule is present within a target cell.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the vector is substantially non-pathogenic and/or substantially non-integrating in a host cell or is substantially non-immunogenic in a host.

In some embodiments, the vector is in an amount sufficient to modulate one or more of phenotype, virus levels, gene expression, compete with other viruses, disease state, etc. at least about 5%, 10%, 15%, 20%, 25%, 30%, 35%, 40%, 45%, 50%, or more.

### Compositions

The synthetic curon or vector described herein may also be included in pharmaceutical compositions with a pharmaceutical excipient, e.g., as described herein. In some embodiments, the pharmaceutical composition comprises at least 10⁵, 10⁶, 10⁷, 10⁸, 10⁹, 10¹⁰, 10¹¹, 10¹², 10¹³, 10¹⁴, or 10¹⁵ synthetic curons. In some embodiments, the pharmaceutical composition comprises about 10⁵-10¹⁵, 105-10¹⁰, or 10¹⁰-10¹⁵ synthetic curons. In some embodiments, the pharmaceutical composition comprises about 10⁸ (e.g., about 10⁵, 10⁶, 10⁷, 10⁸, 10⁹, or 10¹⁰) genomic equivalents/mL of the synthetic curon. In some embodiments, the pharmaceutical composition comprises 10⁵-10¹⁰, 10⁶-10¹⁰, 10⁷-10¹⁰, 10⁸-10¹⁰, 10⁹-10¹⁰, 10⁵-10⁶, 10⁵-10⁷, 10⁵-10⁸, or 10⁵-10⁹ genomic equivalents/mL of the synthetic curon, e.g., as determined according to the method of Example 18. In some embodiments, the pharmaceutical composition comprises sufficient synthetic curons to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the curons per cell to a population of the eukaryotic cells. In some embodiments, the pharmaceutical composition comprises sufficient synthetic curons to deliver at least about 1×10⁴, 1×10⁵, 1×10⁶, 1× or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶- 1×10⁷ copies of a genetic element comprised in the curons per cell to a population of the eukaryotic cells.

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants; or the pharmaceutical composition has low immunogenicity or is substantially non-immunogenic, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), animal-derived components (e.g., serum albumin or trypsin), replication-competent viruses, non-infectious particles, free viral capsid protein, adventitious agents, and aggregates. In embodiments, the contaminant is host cell DNA. In embodiments, the composition comprises less than about 500 ng of host cell DNA per dose. In embodiments, the pharmaceutical composition consists of less than 10% (e.g., less than about 10%, 5%, 4%, 3%, 2%, 1%, 0.5%, or 0.1%) contaminant by weight.

In one aspect, the invention described herein includes a pharmaceutical composition comprising:

a) a synthetic curon comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaceous exterior that is associated with, e.g., envelops or encloses, the genetic element; and

b) a pharmaceutical excipient.

### Vesicles

In some embodiments, the composition further comprises a carrier component, e.g., a microparticle, liposome, vesicle, or exosome. In some embodiments, liposomes comprise spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are generally biocompatible, nontoxic, can deliver both hydrophilic and lipophilic drug molecules, protect their cargo from degradation by plasma enzymes, and transport their load across biological membranes (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

As described herein, additives may be added to vesicles to modify their structure and/or properties. For example, either cholesterol or sphingomyelin may be added to the mixture to help stabilize the structure and to prevent the leakage of the inner cargo. Further, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi: 10.1155/2011/469679 for review). Also, vesicles may be surface modified during or after synthesis to include reactive groups complementary to the reactive groups on the recipient cells. Such reactive groups include without limitation maleimide groups. As an example, vesicles may be synthesized to include maleimide conjugated phospholipids such as without limitation DSPE-MaL-PEG2000.

A vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Formulations made up of phospholipids only are less stable in plasma. However, manipulation of the lipid membrane with cholesterol reduces rapid release of the encapsulated cargo or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In embodiments, lipids may be used to form lipid microparticles. Lipids include, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present invention.

In some embodiments, microparticles comprise one or more solidified polymer(s) that is arranged in a random manner. The microparticles may be biodegradable. Biodegradable microparticles may be synthesized, e.g., using methods known in the art including without limitation solvent evaporation, hot melt microencapsulation, solvent removal, and spray drying. Exemplary methods for synthesizing microparticles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used to form biodegradable microparticles include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water, by surface or bulk erosion.

The microparticles' diameter ranges from 0.1-1000 micrometers (μm). In some embodiments, their diameter ranges in size from 1-750 μm, or from 50-500 μm, or from 100-250 μm. In some embodiments, their diameter ranges in size from 50-1000 μm, from 50-750 μm, from 50-500 μm, or from 50-250 μm. In some embodiments, their diameter ranges in size from 0.05-1000 μm, from 10-1000 μm, from 100-1000 μm, or from 500-1000 μm. In some embodiments, their diameter is about 0.5 μm, about 10 μm, about 50 μm, about 100 μm, about 200 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. As used in the context of microparticle diameters, the term “about” means +/−5% of the absolute value stated.

In some embodiments, a ligand is conjugated to the surface of the microparticle via a functional chemical group (carboxylic acids, aldehydes, amines, sulfhydryls and hydroxyls) present on the surface of the particle and present on the ligand to be attached. Functionality may be introduced into the microparticles by, for example, during the emulsion preparation of microparticles, incorporation of stabilizers with functional chemical groups.

Another example of introducing functional groups to the microparticle is during post-particle preparation, by direct crosslinking particles and ligands with homo- or heterobifunctional crosslinkers. This procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the microparticles may be synthesized to comprise one or more targeting groups on their exterior surface to target a specific cell or tissue type (e.g., cardiomyocytes). These targeting groups include without limitation receptors, ligands, antibodies, and the like. These targeting groups bind their partner on the cells' surface. In some embodiments, the microparticles will integrate into a lipid bilayer that comprises the cell surface and the mitochondria are delivered to the cell.

The microparticles may also comprise a lipid bilayer on their outermost surface. This bilayer may be comprised of one or more lipids of the same or different type. Examples include without limitation phospholipids such as phosphocholines and phosphoinositols. Specific examples include without limitation DMPC, DOPC, DSPC, and various other lipids such as those described herein for liposomes.

In some embodiments, the carrier comprises nanoparticles, e.g., as described herein.

In some embodiments, the vesicles or microparticles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

### Membrane Penetrating Polypeptides

In some embodiments, the composition further comprises a membrane penetrating polypeptide (MPP) to carry the components into cells or across a membrane, e.g., cell or nuclear membrane. Membrane penetrating polypeptides that are capable of facilitating transport of substances across a membrane include, but are not limited to, cell-penetrating peptides (CPPs)(see, e.g., U.S. Pat. No. 8,603,966), fusion peptides for plant intracellular delivery (see, e.g., Ng et al., PLoS One, 2016, 11:e0154081), protein transduction domains, Trojan peptides, and membrane translocation signals (MTS) (see, e.g., Tung et al., Advanced Drug Delivery Reviews 55:281-294 (2003)). Some MPP are rich in amino acids, such as arginine, with positively charged side chains.

Membrane penetrating polypeptides have the ability of inducing membrane penetration of a component and allow macromolecular translocation within cells of multiple tissues in vivo upon systemic administration. A membrane penetrating polypeptide may also refer to a peptide which, when brought into contact with a cell under appropriate conditions, passes from the external environment in the intracellular environment, including the cytoplasm, organelles such as mitochondria, or the nucleus of the cell, in amounts significantly greater than would be reached with passive diffusion.

Components transported across a membrane may be reversibly or irreversibly linked to the membrane penetrating polypeptide. A linker may be a chemical bond, e.g., one or more covalent bonds or non-covalent bonds. In some embodiments, the linker is a peptide linker. Such a linker may be between 2-30 amino acids, or longer. The linker includes flexible, rigid or cleavable linkers.

### Combinations

In one aspect, the synthetic curon or composition comprising a synthetic curon described herein may also include one or more heterologous moiety. In one aspect, the curon or composition comprising a synthetic curon described herein may also include one or more heterologous moiety in a fusion. In some embodiments, a heterologous moiety may be linked with the genetic element. In some embodiments, a heterologous moiety may be enclosed in the proteinaceous exterior as part of the curon. In some embodiments, a heterologous moiety may be administered with the synthetic curon.

In one aspect, the invention includes a cell or tissue comprising any one of the synthetic curons and heterologous moieties described herein.

In another aspect, the invention includes a pharmaceutical composition comprising a synthetic curon and the heterologous moiety described herein.

In some embodiments, the heterologous moiety may be a virus (e.g., an effector (e.g., a drug, small molecule), a targeting agent (e.g., a DNA targeting agent, antibody, receptor ligand), a tag (e.g., fluorophore, light sensitive agent such as KillerRed), or an editing or targeting moiety described herein. In some embodiments, a membrane translocating polypeptide described herein is linked to one or more heterologous moieties. In one embodiment, the heterologous moiety is a small molecule (e.g., a peptidomimetic or a small organic molecule with a molecular weight of less than 2000 daltons), a peptide or polypeptide (e.g., an antibody or antigen-binding fragment thereof), a nanoparticle, an aptamer, or pharmacoagent.

Viruses

In some embodiments, the composition may further comprise a virus as a heterologous moiety, e.g., a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the composition may further comprise a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the composition may further comprise an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus. In some embodiments, the curon is administered with a virus as a heterologous moiety.

In some embodiments, the heterologous moiety may comprise a non-pathogenic, e.g., symbiotic, commensal, native, virus. In some embodiments, the non-pathogenic virus is one or more anelloviruses, e.g., Alphatorquevirus (TT), Betatorquevirus (TTM), and Gammatorquevirus (TTMD). In some embodiments, the anellovirus may include a Torque Teno Virus (TT), a SEN virus, a Sentinel virus, a TTV-like mini virus, a TT virus, a TT virus genotype 6, a TT virus group, a TTV-like virus DXL1, a TTV-like virus DXL2, a Torque Teno-like Mini Virus (TTM), or a Torque Teno-like Midi Virus (TTMD). In some embodiments, the non-pathogenic virus comprises one or more sequences having at least at least about 60%, 70% 80%, 85%, 90% 95%, 96%, 97%, 98% and 99% nucleotide sequence identity to any one of the nucleotide sequences described herein, e.g., Table 19 or Table 20.

In some embodiments, the heterologous moiety may comprise one or more viruses that are identified as lacking in the subject. For example, a subject identified as having dyvirosis may be administered a composition comprising a curon and one or more viral components or viruses that are imbalanced in the subject or having a ratio that differs from a reference value, e.g., a healthy subject.

In some embodiments, the heterologous moiety may comprise one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus. In some embodiments, the curon or the virus is defective, or requires assistance in order to produce infectious particles. Such assistance can be provided, e.g., by using helper cell lines that contain a nucleic acid, e.g., plasmids or DNA integrated into the genome, encoding one or more of (e.g., all of) the structural genes of the replication defective curon or virus under the control of regulatory sequences within the LTR. Suitable cell lines for replicating the curons described herein include cell lines known in the art, e.g., A549 cells, which can be modified as described herein.

Effector

In some embodiments, the composition or synthetic curon may further comprise an effector that possesses effector activity. The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity by triggering increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

Targeting Moiety

In some embodiments, the composition or curon described herein may further comprise a targeting moiety, e.g., a targeting moiety that specifically binds to a molecule of interest present on a target cell. The targeting moiety may modulate a specific function of the molecule of interest or cell, modulate a specific molecule (e.g., enzyme, protein or nucleic acid), e.g., a specific molecule downstream of the molecule of interest in a pathway, or specifically bind to a target to localize the curon or genetic element. For example, a targeting moiety may include a therapeutic that interacts with a specific molecule of interest to increase, decrease or otherwise modulate its function.

Tagging or Monitoring Moiety

In some embodiments, the composition or synthetic curon described herein may further comprise a tag to label or monitor the curon or genetic element described herein. The tagging or monitoring moiety may be removable by chemical agents or enzymatic cleavage, such as proteolysis or intein splicing. An affinity tag may be useful to purify the tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). The tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moiety are combined, in order to connect proteins to multiple other components. The tagging or monitoring moiety may also be removed by specific proteolysis or enzymatic cleavage (e.g. by TEV protease, Thrombin, Factor Xa or Enteropeptidase).

Nanoparticles

In some embodiments, the composition or synthetic curon described herein may further comprise a nanoparticle. Nanoparticles include inorganic materials with a size between about 1 and about 1000 nanometers, between about 1 and about 500 nanometers in size, between about 1 and about 100 nm, between about 50 nm and about 300 nm, between about 75 nm and about 200 nm, between about 100 nm and about 200 nm, and any range therebetween. Nanoparticles generally have a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In nanoparticles described herein, the size limitation can be restricted to two dimensions and so that nanoparticles include composite structure having a diameter from about 1 to about 1000 nm, where the specific diameter depends on the nanoparticle composition and on the intended use of the nanoparticle according to the experimental design. For example, nanoparticles used in therapeutic applications typically have a size of about 200 nm or below.

Additional desirable properties of the nanoparticle, such as surface charges and steric stabilization, can also vary in view of the specific application of interest. Exemplary properties that can be desirable in clinical applications such as cancer treatment are described in Davis et al, Nature 2008 vol. 7, pages 771-782; Duncan, Nature 2006 vol. 6, pages 688-701; and Allen, Nature 2002 vol. 2 pages 750-763, each incorporated herein by reference in its entirety. Additional properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

Small Molecules

In some embodiments, the composition or synthetic curon described herein may further comprise a small molecule. Small molecule moieties include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, the small molecule is an epigenetic modifying agent, for example such as those described in de Groote et al. Nuc. Acids Res. (2012):1-18. Exemplary small molecule epigenetic modifying agents are described, e.g., in Lu et al. J. Biomolecular Screening 17.5(2012):555-71, e.g., at Table 1 or 2, incorporated herein by reference. In some embodiments, an epigenetic modifying agent comprises vorinostat or romidepsin. In some embodiments, an epigenetic modifying agent comprises an inhibitor of class I, II, III, and/or IV histone deacetylase (HDAC). In some embodiments, an epigenetic modifying agent comprises an activator of SirTI. In some embodiments, an epigenetic modifying agent comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (e.e.g, PRMTI), methylstat, 4,4′-dicarboxy-2,2′-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying agent inhibits DNA methylation, e.g., is an inhibitor of DNA methyltransferase (e.g., is 5-azacitidine and/or decitabine). In some embodiments, an epigenetic modifying agent modifies histone modification, e.g., histone acetylation, histone methylation, histone sumoylation, and/or histone phosphorylation. In some embodiments, the epigenetic modifying agent is an inhibitor of a histone deacetylase (e.g., is vorinostat and/or trichostatin A).

In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumour suppressers). One or a combination of molecules from the categories and examples described herein or from (Orme-Johnson 2007, Methods Cell Biol. 2007; 80:813-26) can be used. In one embodiment, the invention includes a composition comprising an antibiotic, anti-inflammatory drug, angiogenic or vasoactive agent, growth factor or chemotherapeutic agent.

Peptides or Proteins

In some embodiments, the composition or synthetic curon described herein may further comprise a peptide or protein. The peptide moieties may include, but are not limited to, a peptide ligand or antibody fragment (e.g., antibody fragment that binds a receptor such as an extracellular receptor), neuropeptide, hormone peptide, peptide drug, toxic peptide, viral or microbial peptide, synthetic peptide, and agonist or antagonist peptide.

Peptides moieties may be linear or branched. The peptide has a length from about 5 to about 200 amino acids, about 15 to about 150 amino acids, about 20 to about 125 amino acids, about 25 to about 100 amino acids, or any range therebetween.

Some examples of peptides include, but are not limited to, fluorescent tags or markers, antigens, antibodies, antibody fragments such as single domain antibodies, ligands and receptors such as glucagon-like peptide-1 (GLP-1), GLP-2 receptor 2, cholecystokinin B (CCKB) and somatostatin receptor, peptide therapeutics such as those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptides useful in the invention described herein also include small antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such small antigen binding peptides may bind a cytosolic antigen, a nuclear antigen, an intra-organellar antigen.

In some embodiments, the composition or curon described herein includes a polypeptide linked to a ligand that is capable of targeting a specific location, tissue, or cell.

Oligonucleotide Aptamers

In some embodiments, the composition or synthetic curon described herein may further comprise an oligonucleotide aptamer. Aptamer moieties are oligonucleotide or peptide aptamers. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers may possess desirable storage properties, and elicit little or no immunogenicity in therapeutic applications.

Both DNA and RNA aptamers can show robust binding affinities for various targets. For example, DNA and RNA aptamers have been selected for t lysozyme, thrombin, human immunodeficiency virus trans-acting responsive element (HIV TAR), (see en.wikipedia.org/wiki/Aptamercite_note-10), hemin, interferon γ, vascular endothelial growth factor (VEGF), prostate specific antigen (PSA), dopamine, and the non-classical oncogene, heat shock factor 1 (HSF1).

Peptide Aptamers

In some embodiments, the composition or synthetic curon described herein may further comprise a peptide aptamer. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide loops of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer loop attached to a transcription factor binding domain is screened against the target protein attached to a transcription factor activating domain. In vivo binding of the peptide aptamer to its target via this selection strategy is detected as expression of a downstream yeast marker gene. Such experiments identify particular proteins bound by the aptamers, and protein interactions that the aptamers disrupt, to cause the phenotype. In addition, peptide aptamers derivatized with appropriate functional moieties can cause specific post-translational modification of their target proteins, or change the subcellular localization of the targets

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### Hosts

The invention is further directed to a host or host cell comprising a synthetic curon described herein. In some embodiments, the host or host cell is a plant, insect, bacteria, fungus, vertebrate, mammal (e.g., human), or other organism or cell. In certain embodiments, as confirmed herein, provided curons infect a range of different host cells. Target host cells include cells of mesodermal, endodermal, or ectodermal origin. Target host cells include, e.g., epithelial cells, muscle cells, white blood cells (e.g., lymphocytes), kidney tissue cells, lung tissue cells.

In some embodiments, the curon is substantially non-immunogenic in the host. The curon or genetic element fails to produce an undesired substantial response by the host's immune system. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., infected with) a synthetic curon. In some embodiments, the host is a mammal, such as a human. The amount of the curon in the host can be measured at any time after administration. In certain embodiments, a time course of curon growth in a culture is determined.

In some embodiments, the curon, e.g., a curon as described herein, is heritable. In some embodiments, the curon is transmitted linearly in fluids and/or cells from mother to child. In some embodiments, daughter cells from an original host cell comprise the curon. In some embodiments, a mother transmits the curon to child with an efficiency of at least 25%, 50%, 60%, 70%, 80%, 85%, 90%, 95%, or 99%, or a transmission efficiency from host cell to daughter cell at least 25%, 50%, 60%, 70%, 80%, 85%, 90%, 95%, or 99%. In some embodiments, the curon in a host cell has a transmission efficiency during meiosis of at 25%, 50%, 60%, 70%, 80%, 85%, 90%, 95%, or 99%. In some embodiments, the curon in a host cell has a transmission efficiency during mitosis of at least 25%, 50%, 60%, 70%, 80%, 85%, 90%, 95%, or 99%. In some embodiments, the curon in a cell has a transmission efficiency between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween.

In some embodiments, the curon, e.g., synthetic curon replicates within the host cell. In one embodiment, the synthetic curon is capable of replicating in a mammalian cell, e.g., human cell.

While in some embodiments the synthetic curon replicates in the host cell, the synthetic curon does not integrate into the genome of the host, e.g., with the host's chromosomes. In some embodiments, the synthetic curon has a negligible recombination frequency, e.g., with the host's chromosomes. In some embodiments, the curon has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### Methods of Use

The synthetic curons and compositions comprising synthetic curons described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. The synthetic curons may be administered alone or formulated as a pharmaceutical composition.

The synthetic curons may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of a synthetic curon or composition comprising same, e.g., as described herein, may result in delivery of a genetic element comprised by the synthetic curon to a target cell, e.g., in a subject.

A synthetic curon or composition thereof described herein, e.g., comprising an exogenous effector or payload, may be used to deliver the exogenous effector or payload to a cell, tissue, or subject. In some embodiments, the synthetic curon or composition thereof is used to deliver the exogenous effector or payload to bone marrow, blood, heart, GI or skin. Delivery of an exogenous effector or payload by administration of a synthetic curon composition described herein may modulate (e.g., increase or decrease) expression levels of a noncoding RNA or polypeptide in the cell, tissue, or subject. Modulation of expression level in this fashion may result in alteration of a functional activity in the cell to which the exogenous effector or payload is delivered. In some embodiments, the modulated functional activity may be enzymatic, structural, or regulatory in nature.

In some embodiments, the synthetic curon, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, a synthetic curon or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the synthetic curon or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

Examples of diseases, disorders, and conditions that can be treated with the synthetic curon described herein, or a composition comprising the synthetic curon, include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the synthetic curon modulates (e.g., increases or decreases) an activity or function in a cell with which the curon is contacted. In some embodiments, the synthetic curon modulates (e.g., increases or decreases) the level or activity of a molecule (e.g., a nucleic acid or a protein) in a cell with which the curon is contacted. In some embodiments, the synthetic curon decreases viability of a cell, e.g., a cancer cell, with which the curon is contacted, e.g., by at least about 10%, 20%, 30%, 40%, 50%, 60%, 70%, 80%, 90%, 95%, 99%, or more. In some embodiments, the synthetic curon comprises an effector, e.g., an miRNA, e.g., miR-625, that decreases viability of a cell, e.g., a cancer cell, with which the curon is contacted, e.g., by at least about 10%, 20%, 30%, 40%, 50%, 60%, 70%, 80%, 90%, 95%, 99%, or more. In some embodiments, the synthetic curon increases apoptosis of a cell, e.g., a cancer cell, e.g., by increasing caspase-3 activity, with which the curon is contacted, e.g., by at least about 10%, 20%, 30%, 40%, 50%, 60%, 70%, 80%, 90%, 95%, 99%, or more. In some embodiments, the synthetic curon comprises an effector, e.g., an miRNA, e.g., miR-625, that increases apoptosis of a cell, e.g., a cancer cell, e.g., by increasing caspase-3 activity, with which the curon is contacted, e.g., by at least about 10%, 20%, 30%, 40%, 50%, 60%, 70%, 80%, 90%, 95%, 99%, or more.

### Additional Curon Embodiments

In one aspect, the invention includes a synthetic curon comprising: a genetic element comprising (i) a sequence encoding a non-pathogenic exterior protein, (ii) an exterior protein binding sequence that binds the genetic element to the non-pathogenic exterior protein, and (iii) a sequence encoding an effector, e.g., a regulatory nucleic acid; and a proteinaceous exterior that is associated with, e.g., envelops or encloses, the genetic element.

In one aspect, the invention includes a pharmaceutical composition comprising: a) a curon comprising: a genetic element comprising (i) a sequence encoding a non-pathogenic exterior protein, (ii) an exterior protein binding sequence that binds the genetic element to the non-pathogenic exterior protein, and (iii) a sequence encoding an effector, e.g., a regulatory nucleic acid; and a proteinaceous exterior that is associated with, e.g., envelops or encloses, the genetic element; and b) a pharmaceutical excipient.

In various aspects of the invention delineated herein, one or more of the various embodiments described herein may be combined.

In some embodiments, curon or composition described herein further comprises at least one of the following characteristics: the genetic element is a single-stranded DNA; the genetic element is circular; the curon is non-integrating; the curon has a sequence, structure, and/or function based on an anellovirus or other non-pathogenic virus, and the curon is non-pathogenic.

In some embodiments, the proteinaceous exterior comprises the non-pathogenic exterior protein. In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges. In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is non-immunogenic or non-pathogenic in a host. For example, data provided herein confirm that provided curons are infectious.

In some embodiments, the sequence encoding the non-pathogenic exterior protein comprise a sequence at least 70%, 80%, 85%, 90%, 95%, 96%, 97%, 98%, 99% identical to one or more sequences or a fragment thereof listed in Table 15. In some embodiments, the non-pathogenic exterior protein comprises a sequence at least 70%, 80%, 85%, 90%, 95%, 96%, 97%, 98%, 99% identical to one or more sequences or a fragment thereof listed in Table 16 or Table 17. In some embodiments, the non-pathogenic exterior protein comprises at least one functional domain that provides one or more functions, e.g., species and/or tissue and/or cell tropism, viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection.

In some embodiments, the effector comprises a regulatory nucleic acid, e.g., an miRNA, siRNA, mRNA, lncRNA, RNA, DNA, an antisense RNA, gRNA; a therapeutic, e.g., fluorescent tag or marker, antigen, peptide therapeutic, synthetic or analog peptide from naturally-bioactive peptide, agonist or antagonist peptide, anti-microbial peptide, pore-forming peptide, a bicyclic peptide, a targeting or cytotoxic peptide, a degradation or self-destruction peptide, and degradation or self-destruction peptides, small molecule, immune effector (e.g., influences susceptibility to an immune response/signal), a death protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component. In some embodiments, the effector comprises a sequence at least 70%, 80%, 85%, 90%, 95%, 96%, 97%, 98%, 99% identical to one or more miRNA sequences listed in Table 18. In some embodiments, the effector, e.g., miRNA, targets a host gene, e.g., modulates expression of the gene.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), a sequence that encodes a therapeutic mRNA or protein, and a sequence that encodes a cytolytic/cytotoxic RNA or protein. In some embodiments, the genetic element has one or more of the following characteristics: is non-integrating with a host cell's genome, is an episomal nucleic acid, is a single stranded DNA, is about 1 to 10 kb, exists within the nucleus of the cell, is capable of being bound by endogenous proteins, and produces a microRNA that targets host genes.

In some embodiments, the genetic element comprises at least one viral sequence or at least 70%, 80%, 85%, 90%, 95%, 96%, 97%, 98%, 99% identity to one or more sequences or a fragment thereof listed in Table 19 or Table 20. In one such embodiment, the viral sequence is from at least one of a single stranded DNA virus (e.g., Anellovirus, Bidnavirus, Circovirus, Geminivirus, Genomovirus, Inovirus, Microvirus, Nanovirus, Parvovirus, and Spiravirus), a double stranded DNA virus (e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus), a RNA virus (e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus). In another embodiment, the viral sequence is from one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus.

In some embodiments, the protein binding sequence interacts with the arginine-rich region of the proteinaceous exterior.

In some embodiments, the curon is capable of replicating in a mammalian cell, e.g., human cell. In some embodiments, the curon is substantially non-pathogenic and/or non-integrating in a host cell. In some embodiments, the curon is substantially non-immunogenic in a host. In some embodiments, the curon inhibits/enhances one or more viral properties, e.g., tropism, e.g., infectivity, e.g., immunosuppression/activation, in a host or host cell. In some embodiments, the curon is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more).

In some embodiments, the composition further comprises at least one virus or vector comprising a genome of the virus, e.g., a variant of the curon, e.g., a commensal/native virus. In some embodiments, the composition further comprises a heterologous moiety, e.g., at least one small molecule, antibody, polypeptide, nucleic acid, targeting agent, imaging agent, nanoparticle, and a combination thereof.

In one aspect, the invention includes a vector comprising a genetic element comprising (i) a sequence encoding a non-pathogenic exterior protein, (ii) an exterior protein binding sequence that binds the genetic element to the non-pathogenic exterior protein, and (iii) a sequence encoding an effector, e.g., a regulatory nucleic acid.

In various aspects of the invention delineated herein, one or more of the various embodiments described herein may be combined.

In some embodiments, the genetic element fails to integrate with a host cell's genome. In some embodiments, the genetic element is capable of replicating in a mammalian cell, e.g., human cell.

In some embodiments, the vector further comprises an exogenous nucleic acid sequence, e.g., selected to modulate expression of a gene, e.g., a human gene.

In one aspect, the invention includes a pharmaceutical composition comprising the vector described herein and a pharmaceutical excipient.

In various aspects of the invention delineated herein, one or more of the various embodiments described herein may be combined.

In some embodiments, the vector is substantially non-pathogenic and/or non-integrating in a host cell. In some embodiments, the vector is substantially non-immunogenic in a host.

In some embodiments, the vector is in an amount sufficient to modulate (phenotype, virus levels, gene expression, compete with other viruses, disease state, etc. at least about 5%, 10%, 15%, 20%, 25%, 30%, 35%, 40%, 45%, 50%, or more).

In some embodiments, the composition further comprises at least one virus or vector comprising a genome of the virus, e.g., a variant of the curon, a commensal/native virus, a helper virus, a non-anellovirus. In some embodiments, the composition further comprises a heterologous moiety, at least one small molecule, antibody, polypeptide, nucleic acid, targeting agent, imaging agent, nanoparticle, and a combination thereof.

In one aspect, the invention includes a method of producing, propagating, and harvesting the curon described herein.

In one aspect, the invention includes a method of designing and making the vector described herein.

In one aspect, the invention includes a method of identifying dysvirosis in a subject comprising: analyzing genetic information from a sample obtained from a subject in need thereof, wherein viral genetic information is isolated from the subject's genetic information and other microorganisms; comparing the viral genetic information to a reference, e.g., a control, a healthy subject; and identifying dysvirosis in the subject if comparison of the viral genetic information yields an imbalance or irregular ratio of viral genetic information in the subject.

In various aspects of the invention delineated herein, one or more of the various embodiments described herein may be combined.

In some embodiments, the subject is administered the pharmaceutical composition further comprising one or more viral strains that are not represented in the viral genetic information. In some embodiments, the subject has inflammatory condition or disorder, autoimmune condition or disease, chronic/acute condition or disorder, cancer, gastrointestinal condition or disorder, or any combination thereof.

In embodiments, the synthetic curon inhibits interferon expression.

### Methods of Production

### Producing the Genetic Element

Methods of making the genetic element of the curon are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

In some embodiments, the genetic element may be designed using computer-aided design tools. The curon may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the curon. The segments or ORFs may be assembled into the curon, e.g., in vitro recombination or unique restriction sites at 5′ and 3′ ends to enable ligation.

The genetic element can alternatively be synthesized with a design algorithm that parses the curon into oligo-length fragments, creating optimal design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element or segment of the genetic element may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. AnyDot.chips and methods for using them are described in part in International Publication Application Nos. WO 02088382, WO 03020968, WO 0303 1947, WO 2005044836, PCTEP 05105657, PCMEP 05105655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. The sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishably type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In some embodiments, shotgun sequencing is performed. In shotgun sequencing, DNA is broken up randomly into numerous small segments, which are sequenced using the chain termination method to obtain reads. Multiple overlapping reads for the target DNA are obtained by performing several rounds of this fragmentation and sequencing. Computer programs then use the overlapping ends of different reads to assemble them into a continuous sequence.

### Producing the Synthetic Curon

The genetic elements and vectors comprising the genetic elements prepared as described herein can be used in a variety of ways to express the synthetic curon in appropriate host cells. In some embodiments, the genetic element and vectors comprising the genetic element are transfected in appropriate host cells and the resulting RNA may direct the expression of the curon gene products, e.g., non-pathogenic protein and protein binding sequence, at high levels. Host cell systems which provide for high levels of expression include continuous cell lines that supply viral functions, such as cell lines superinfected with APV or MPV, respectively, cell lines engineered to complement APV or MPV functions, etc.

In some embodiments, the synthetic curon is produced as described in any of Examples 1, 2, 5, 6, or 15-17.

In some embodiments, the synthetic curon is cultivated in continuous animal cell lines in vitro. According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST. Also, other mammalian cells likes are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines susceptible to infection with curons include, but are not limited to cell lines of human or primate origin, such as human or primate kidney carcinoma cell lines.

In some embodiments, the genetic elements and vectors comprising the genetic elements are transfected into cell lines that express a viral polymerase protein in order to achieve expression of the curon. To this end, transformed cell lines that express a curon polymerase protein may be utilized as appropriate host cells. Host cells may be similarly engineered to provide other viral functions or additional functions.

To prepare the synthetic curon disclosed herein, a genetic element or vector comprising the genetic element disclosed herein may be used to transfect cells which provide curon proteins and functions required for replication and production. Alternatively, cells may be transfected with helper virus before, during, or after transfection by the genetic element or vector comprising the genetic element disclosed herein. In some embodiments, a helper virus may be useful to complement production of an incomplete viral particle. The helper virus may have a conditional growth defect, such as host range restriction or temperature sensitivity, which allows the subsequent selection of transfectant viruses. In some embodiments, a helper virus may provide one or more replication proteins utilized by the host cells to achieve expression of the curon. In some embodiments, the host cells may be transfected with vectors encoding viral proteins such as the one or more replication proteins.

The genetic element or vector comprising the genetic element disclosed herein can be replicated and produced into curon particles by any number of techniques known in the art, as described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

The production of curon-containing cell cultures according to the present invention can be carried out in different scales, such as in flasks, roller bottles or bioreactors. The media used for the cultivation of the cells to be infected are known to the skilled person and will comprise the standard nutrients required for cell viability but may also comprise additional nutrients dependent on the cell type. Optionally, the medium can be protein-free. Depending on the cell type the cells can be cultured in suspension or on a substrate.

The purification and isolation of synthetic curons can be performed according to methods known by the skilled person in virus production and is described for example by Rinaldi, et al., DNA Vaccines: Methods and Protocols (Methods in Molecular Biology), 3rd ed. 2014, Humana Press.

In one aspect, the present invention includes a method for the in vitro replication and propagation of the curon as described herein, which may comprise the following steps: (a) transfecting a linearized genetic element into a cell line sensitive to curon infection; (b) harvesting the cells and isolating cells showing the presence of the genetic element; (c) culturing the cells obtained in step (b) for at least three days, such as at least one week or longer, depending on experimental conditions and gene expression; and (d) harvesting the cells of step (c).

### Administration/Delivery

The composition (e.g., a pharmaceutical composition comprising a synthetic curon as described herein) may be formulated to include a pharmaceutically acceptable excipient. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering a curon to a subject. The method includes administering a pharmaceutical composition comprising a curon as described herein to the subject. In some embodiments, the administered curon replicates in the subject (e.g., becomes a part of the virome of the subject).

In one aspect, the invention features a method of administering a curon to a subject with dysvirosis. The method includes selecting a subject having dysvirosis as described herein, and administering a pharmaceutical composition comprising a curon as described herein to the subject. In some embodiments, the administered curon replicates in the subject (e.g., becomes a part of the virome of the subject).

The pharmaceutical composition may include wild-type or native viral elements and/or modified viral elements. The curon may include one or more of the sequences (e.g., nucleic acid sequences or nucleic acid sequences encoding amino acid sequences thereof) in any of Tables 1-20 or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any of Tables 1-20. The curon may encode one or more of the sequences in any of Tables 1-20 or a sequence with at least about 60%, 65%, 70%, 75%, 80%, 85%, 90% 95%, 96%, 97%, 98% and 99% sequence identity to any one of the amino acid sequences in any of Tables 2, 4, 6, 8, 10, 12, 14, or 16. The curon may include one or more of the sequences in Table 19 or Table 20 or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in Table 19 or Table 20.

In some embodiments, the synthetic curon is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the synthetic curon is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the synthetic curon inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In one aspect, the invention includes a method of identifying dysvirosis, e.g., dysregulation of viral populations present within a host, in a subject comprising analyzing genetic information from a sample obtained from a subject in need thereof, wherein viral genetic information is isolated from the subject's genetic information and other microorganisms; comparing the viral genetic information to a reference, e.g., a control, a healthy subject; and identifying dysvirosis in the subject if comparison of the viral genetic information yields an imbalance or irregular ratio of viral genetic information in the subject.

In one aspect, the present invention also includes a method for generating a database of genetic information for identifying dysviriosis in a diseased subject, which may comprise the following steps (i) determining nucleotide sequences of a host cell genome in a sample from a healthy subject; (ii) determining viral nucleic acid sequences present in the host cell genome and/or present in episomal form; (iii) compiling a database of the viral nucleic acid sequences determined in step (ii) associated with a specific viral strain; and (iv) repeat steps (i)-(iii) for a plurality of subjects to populate the database.

In one aspect, the invention includes a method of administering the pharmaceutical composition described herein to a subject with dysvirosis, comprising obtaining the viral genetic information as described herein and administering a pharmaceutical composition comprising the curon described herein in a dose sufficient to alter a virome within the subject, e.g., at least about 5%, 10%, 15%, 20%, 25%, 30%, 35%, 40%, 45%, 50%, or more as compared to a reference, e.g., a healthy control.

In some embodiments, the subject is administered the pharmaceutical composition further comprising one or more viral strains that are not represented in the viral genetic information.

In some embodiments, the pharmaceutical composition comprising a curon described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the curon is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the curon is sufficient to compete with chronic or acute viral infection. In certain embodiments, the curon may be administered prophylactically to protect from viral infections (e.g. a provirotic). In some embodiments, the curon is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Example 1: Preparation of Curons

This example describes the design and synthesis of a synthetic curon that inhibits interferon (IFN) expression.

A curon (Curon A) is designed starting with 1) a DNA sequence for a capsid gene encoding a non-pathogenic packaging enclosure (Arch Virol (2007) 152: 1961-1975), Accession Number: A7XCE8.1 (ORF11_TTW3); 2) a DNA sequence coding for a microRNA that targets a host gene (e.g. IFN) (PLOS Pathogen (2013), 9(12), e1003818), Accession number: AJ620231.1; and 3) a DNA sequence (Journal of Virology (2003), 77(24), 13036-13041) that binds to a specific region in the capsid protein, (e.g., specific region of capsid having an Accession Number: Q99153.1).

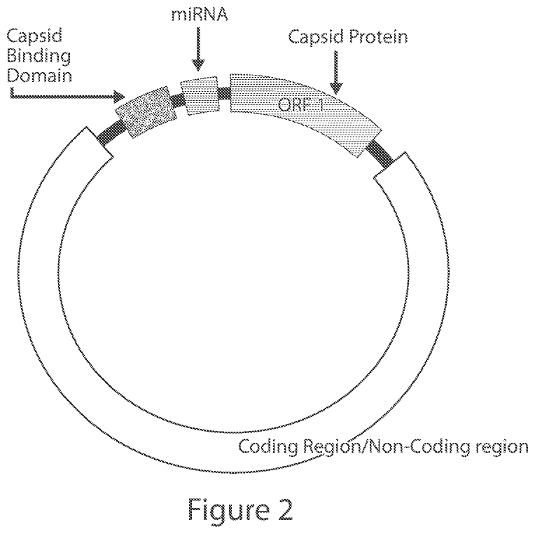

To this sequence is added 1 kb non-coding DNA sequences (Curon B). The designed curon (FIG. 2) is chemically synthesized into 3 kb (total size), which is sequence verified.

The curon sequence is transfected into human embryonic kidney 293T cells (1 mg per 10⁵ cells on 12-well plates) with JetPEI reagent (PolyPlus-transfection, Illkirch, France) as recommended by the manufacturer. Controls transfections are included with vector alone or cells transfected with JetPEI alone and transfection efficiencies are optimized with a reporter plasmid encoding GFP. Fluorescence of control transfections is measured to ensure properly transfected cells. Transfected cultures are incubated overnight at 37° C. and 5% carbon dioxide.

After 18 hrs, the cells are washed three times with PBS before adding fresh medium. The supernatant is collected for ultracentrifugation and harvest of curons as follows. The medium is cleared by centrifugation at 4,000×g for 30 min and then at 8,000×g for 15 min to remove cells and cell debris. The supernatant is then filtered through 0.45-nm-pore-size filters. Curons are pelleted at 27,000 rpm for 1 hr through a 5% sucrose cushion (5 ml) and resuspended in 1× phosphate-buffered saline (PBS) plus 0.1% bacitracin in 1/100 of the original volume. The concentrated Curons are centrifuged through a 20 to 35% sucrose step gradient at 24,000 rpm for 2 hr. The curon band at the gradient junction is collected. The curons are then diluted with 1×PBS and pelleted at 27,000 rpm for 1 hr. The Curon pellets are resuspended in 1×PBS and further purified through a 20 to 35% continuous sucrose gradient.

### Example 2: Large-Scale Production of Curons (Curon A and/or B)

This example describes production and propagation of curons.

Purified curons as described in Example 1 are prepared for large-scale amplification in spinner flasks with producer A549 cells grown in suspension. A549 cells are maintained in F12K medium, 10% fetal bovine serum, 2 mM glutamine and antibiotics. A549 cells are infected with curons at a curon load of 10⁶ curons to produce ˜1×10⁷ curon particles after an incubation at 37° C. and 5% carbon dioxide for 24 hrs. Cells are then washed three times with PBS and incubated with fresh medium for 6 hrs.

For curon purification, two ultracentrifugation steps based on cesium chloride gradients are performed followed by dialysis as follows (Bio-Protocol (2012) Bio101: e201). Cells are removed by centrifugation (6000×g for 10 min) and the supernatant is filtered through 0.8 and then 0.2 μm filters. The filtrate is concentrated by passage through filter membranes (100,000 mw) to a volume of 8 ml. The retentate is loaded into a cesium sulfate solution and centrifuged at 247,000×g for 20 h. Curon bands are removed, placed into 14,000 mw cutoff dialysis tubing, and dialyzed. A further concentration may be performed, if desired.

### Example 3: Effects of Curons In Vitro (Curon A)

This example describes in vitro assessment of expression and effector function, e.g., expression of the miRNA, of the curon after cell infection.

The effect of purified curons as described in Example 1 is assessed in vitro through endogenous gene regulation (e.g. IFN signaling). HEK293T cells are co-transfected with dual luciferase plasmids (firefly luciferase with an interferon-stimulated response element (ISRE) based promoter and transfection control Renilla luciferase with constitutive promoter): Luciferase reporter mix (pcDNA3.1dsRluc to pISRE-Luc at 1:4 ratio (Clonetech)) (J Virol (2008), 82: 9823-9828).

Curons are administered at multiplicity of infection of 10⁷ to HEK293T cells seeded in a 6-well plate (2 sets of triplicates-3 control wells and 3 experimental wells with Curon A).

After 48 hours, the media is replaced with new media with or without 100 u/ml of universal type I interferon (PBL, Piscataway, N.J.). Sixteen hours after IFN treatment, a dual-luciferase assay (J Virol (2008), 82: 9823-9828) is performed to determine IFN signaling. Firefly luciferase is normalized to Renilla luciferase expression to control for transfection differences. The fold induction of the ISRE ffLuc reporter is calculated by dividing the comparable experimental wells by the control wells and induction of each condition is compared relative to the negative control.

In an embodiment, a decreased luciferase signal in the curon treatment group compared to a control will indicate that the curons decrease IFN production in the cells.

### Example 4: Immunologic Effects of Curons (Curon A)

This example describes in vivo effector function, e.g., expression of the miRNA, of the curon after administration.

Purified curons prepared as described in Examples 1 and 2 are intravenously administered to healthy pigs at various doses using hundred-fold dilutions starting from 10¹⁴ genome equivalents per kilogram down to 0 genome equivalents per kilogram. In order to evaluate the effects on immune tolerance, pigs are injected daily for 3 days with the dosages of curons specified above or vehicle control PBS and sacrificed after 3 days.

Spleen, bone marrow and lymph nodes are harvested. Single cell suspensions are prepared from each of the tissues and stained with extracellular markers for MHC-II, CD11c, and intracellular IFN. MHC+, CD11c+, IFN+ antigen presenting cells are analyzed via flow cytometry from each tissue, e.g., wherein a cell that is positive for a given one of the above-mentioned markers is a cell that exhibits higher fluorescence than 99% of cells in a negative control population that lack expression of the marker but is otherwise similar to the assay population of cells, under the same conditions.

In an embodiment, a decreased number of IFN+ cells in the curon treatment group compared to the control will indicate that the curons decrease IFN production in cells after administration.

### Example 5: Preparation of Synthetic Curons

This example demonstrates in vitro production of a synthetic curon.

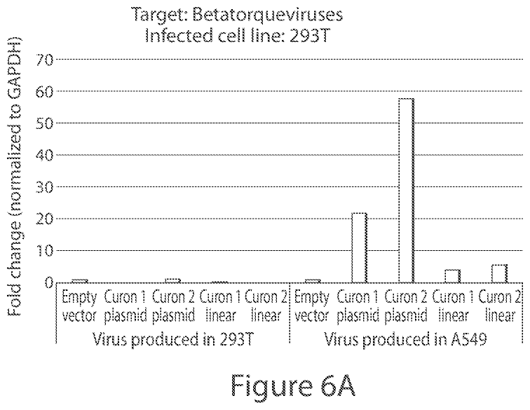

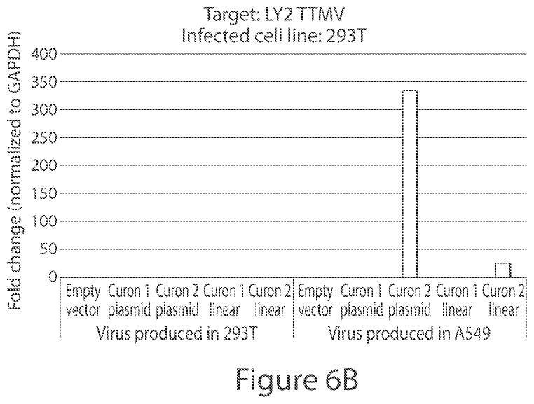

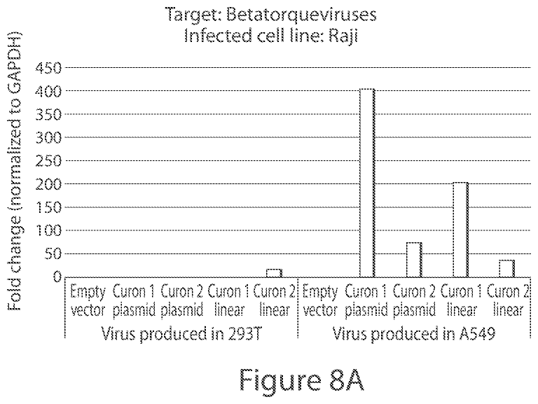

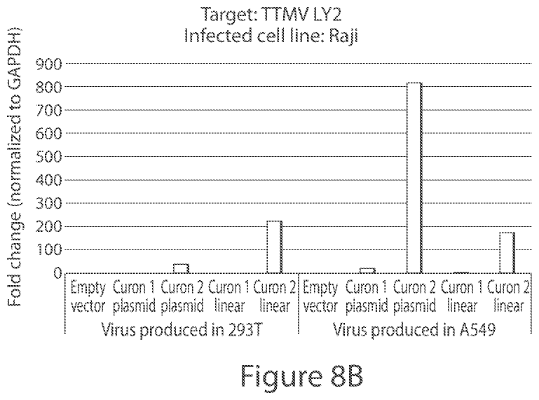

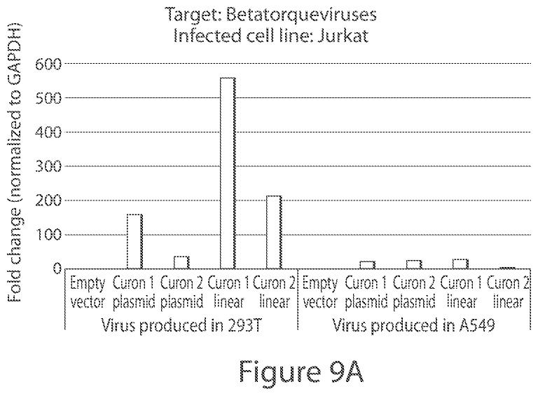

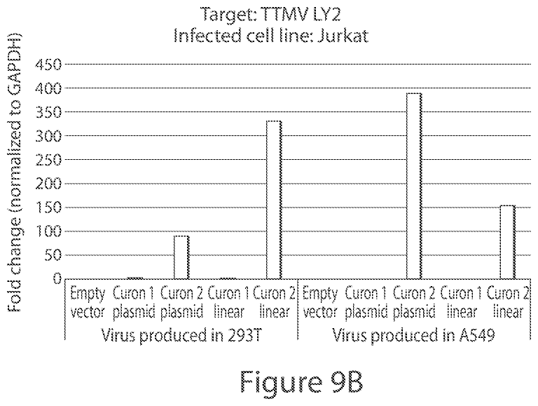

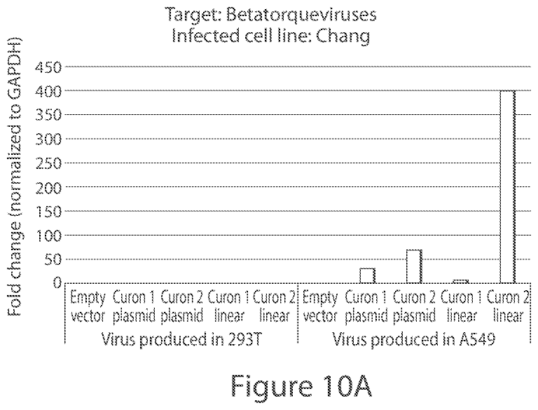

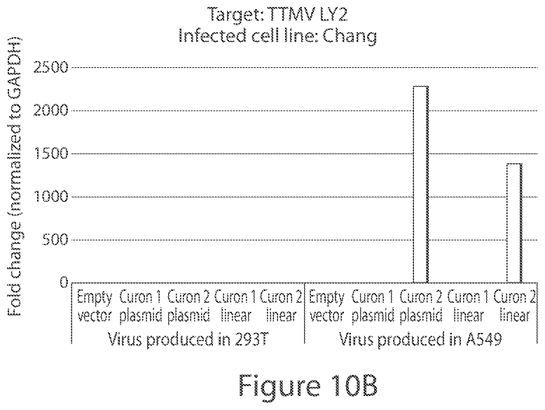

DNA sequences from LY1 and LY2 strains of TTMiniV (Eur Respir J. 2013 August; 42(2):470-9), between the EcoRV restriction enzyme sites, were cloned into a kanamycin vector (Integrated DNA Technologies). Curons including DNA sequences from the LY1 and LY2 strains of TTMiniV are referred to as Curon 1 and Curon 2 respectively, in Examples 6 and 7 and in FIGS. 6A-10B. Cloned constructs were transformed into 10-Beta competent E. coli. (New England Biolabs Inc.), followed by plasmid purification (Qiagen) according to the manufacturer's protocol.

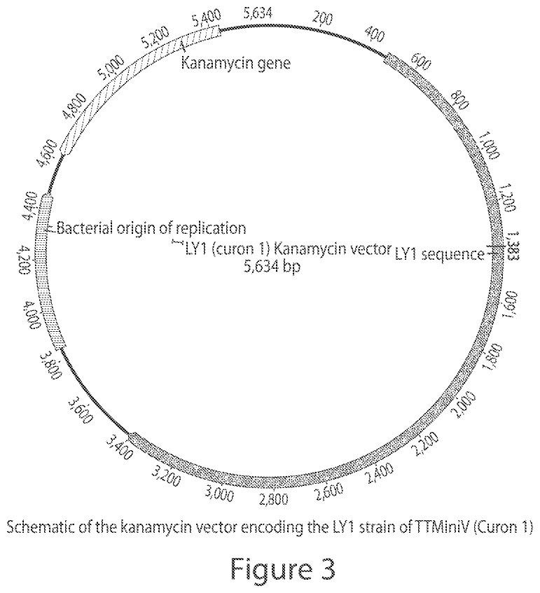

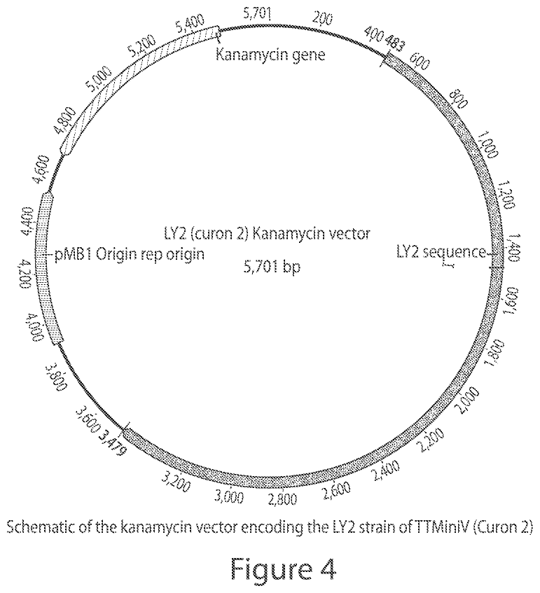

DNA constructs (FIG. 3 and FIG. 4) were linearized with EcoRV restriction digest (New England Biolabs, Inc.) at 37 degree Celsius for 6 hours, followed by agarose gel electrophoresis, excision of a correctly size DNA band (2.9 kilobase pairs), and gel purification of DNA from excised agarose bands using a gel extraction kit (Qiagen) according to the manufacturer's protocol.

### Example 6: Assembly and Infection of Curons

This example demonstrates successful in vitro production of infectious curons using synthetic DNA sequences as described in Example 5.

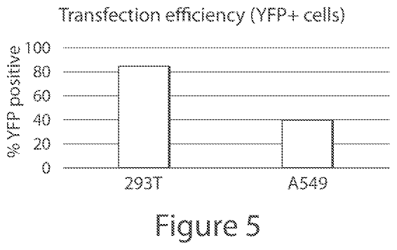

Curon DNA (obtained in Example 5) was transfected into either HEK293T cells (human embryonic kidney cell line) or A549 cells (human lung carcinoma cell line), either in an intact plasmid or in linearized form, with lipid transfection reagent (Thermo Fisher Scientific). 6 ug of plasmid or 1.5 ug of linearized DNA was used for transfection of 70% confluent cells in T25 flasks. Empty vector backbone lacking the viral sequences included in the curon was used as a negative control. Six hours post-transfection, cells were washed with PBS twice and were allowed to grow in fresh growth medium at 37 degrees Celsius and 5% carbon dioxide. DNA sequences encoding the human Ef1alpha promoter followed by YFP gene were synthesized from IDT. This DNA sequence was blunt end ligated into a cloning vector (Thermo Fisher Scientific). The resulting vector was used as a control to assess transfection efficiency. YFP was detected using a cell imaging system (Thermo Fisher Scientific) 72 hours post transfection. The transfection efficiencies of HEK293T and A549 cells were calculated as 85% and 40% respectively (FIG. 5).

Supernatants of 293T and A549 cells transfected with curons were harvested 96 hours post transfection. The harvested supernatants were spun down at 2000 rpm for 10 minutes at 4 degrees Celsius to remove any cell debris. Each of the harvested supernatants was used to infect new 293T and A549 cells, respectively, that were 70% confluent in wells of 24 well plates. Supernatants were washed away after 24 hours of incubation at 37 degrees Celsius and 5% carbon dioxide, followed by two washes of PBS, and replacement with fresh growth medium. Following incubation of these cells at 37 degrees and 5% carbon dioxide for another 48 hours, cells were individually harvested for genomic DNA extraction. Genomic DNA from each of the samples was harvested using a genomic DNA extraction kit (Thermo Fisher Scientific), according to manufacturer's protocol.

To confirm the successful infection of 293T and A549 cells by curons produced in vitro, 100 ng of genomic DNA harvested as described herein was used to perform quantitative polymerase chain reaction (qPCR) using primers specific for beta-torque viruses or LY2 specific sequences. SYBR green reagent (Thermo Fisher Scientific) was used to perform qPCR, as per manufacturer's protocol. qPCR for primers specific to genomic DNA sequence of GAPDH was used for normalization. The sequences for all the primers used are listed in Table 21.

TABLE 21

| | Primer sequence (5′ > 3′) | |
|---|---|---|
| Target | Forward | Reverse |
| Betatorqueviruses | ATTCGAATGGCTGAGTTTATGC | CCTTGACTACGGTGGTTTCAC |
| | (SEQ ID NO: 690) | (SEQ ID NO: 693) |
| LY2 TTMiniV | CACGAATTAGCCAAGACTGGGCAC | TGCAGGCATTCGAGGGCTTGTT |
| strain | (SEQ ID NO: 691) | (SEQ ID NO: 694) |
| GAPDH | GCTCCCACTCCTGATTTCTG | TTTAACCCCCTAGTCCCAGG |
| | (SEQ ID NO: 692) | (SEQ ID NO: 695) |

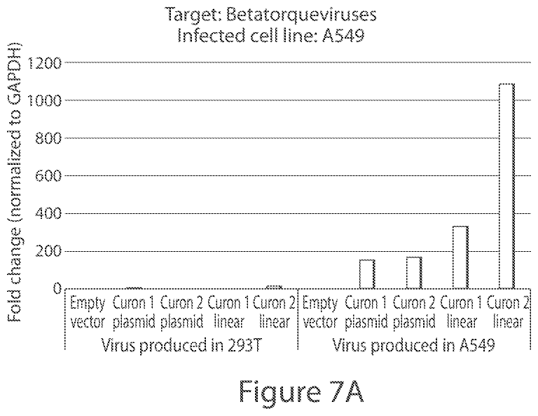

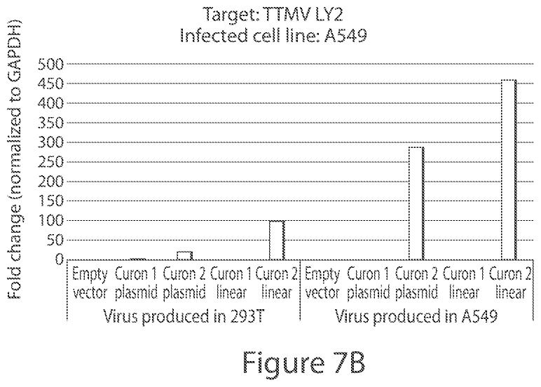

As shown in the qPCR results depicted in FIGS. 6A, 6B, 7A, and 7B, the curons produced in vitro and as described in this example were infectious.

### Example 7: Selectivity of Curons

This example demonstrates the ability of synthetic curons produced in vitro to infect cell lines of a variety of tissue origins.

Supernatants with the infectious TTMiniV curons (described in Example 5) were incubated with 70% confluent 293T, A549, Jurkat (an acute T cell leukemia cell line), Raji (a Burkitt's lymphoma B cell line), and Chang (a liver carcinoma cell line) cell lines at 37 degrees and 5% carbon dioxide in wells of 24 well plates. Cells were washed with PBS twice, 24 hours post infection, followed by replacement with fresh growth medium. Cells were then incubated again at 37 degrees and 5% carbon dioxide for another 48 hours, followed by harvest for genomic DNA extraction. Genomic DNA from each of the samples was harvested using a genomic DNA extraction kit (Thermo Fisher Scientific), according to manufacturer's protocol.

To confirm successful infection of these cell lines by curons produced in the previous Example, 100 ng of genomic DNA harvested as described herein was used to perform quantitative polymerase chain reaction (qPCR) using primers specific for beta-torqueviruses or LY2 specific sequences. SYBR green reagent (Thermo Fisher Scientific) was used to perform qPCR, as per manufacturer's protocol. qPCR for primers specific to genomic DNA sequence of GAPDH was used for normalization. The sequences for all the primers used are listed in Table 21.

As shown in the qPCR results depicted in FIGS. 6A-10B, not only were curons produced in vitro infectious, they were able to infect a variety of cell lines, including examples of epithelial cells, lung tissue cells, liver cells, carcinoma cells, lymphocytes, lymphoblasts, T cells, B cells, and kidney cells. It was also observed that a synthetic curon was able to infect HepG2 cells, resulting in a greater than 100-fold increase relative to a control.

### Example 8: Identification and Use of Protein Binding Sequences

This example describes putative protein-binding sites in the Anellovirus genome, which can be used for amplifying and packaging effectors, e.g., in a curon as described herein. In some instances, the protein-binding sites may be capable of binding to an exterior protein, such as a capsid protein.

Two conserved domains within the Anellovirus genome are putative origins of replication: the 5′ UTR conserved domain (5CD) and the GC-rich domain (GCR) (de Villiers et al., Journal of Virology 2011; Okamoto et al., Virology 1999). In one example, in order to confirm whether these sequences act as DNA replication sites or as capsid packaging signals, deletions of each region are made in plasmids harboring TTMV-LY2. A539 cells are transfected with pTTMV-LY2Δ5CD or pTTMV-LY2ΔGCR. Transfected cells are incubated for four days, and then virus is isolated from supernatant and cell pellets. A549 cells are infected with virus, and after four days, virus is isolated from the supernatant and infected cell pellets. qPCR is performed to quantify viral genomes from the samples. Disruption of an origin of replication prevents viral replicase from amplifying viral DNA and results in reduced viral genomes isolated from transfected cell pellets compared to wild-type virus. A small amount of virus is still packaged and can be found in the transfected supernatant and infected cell pellets. In some embodiments, disruption of a packaging signal will prevent the viral DNA from being encapsulated by capsid proteins. Therefore, in embodiments, there will still be an amplification of viral genomes in the transfected cells, but no viral genomes are found in the supernatant or infected cell pellets.

In a further example, in order to characterize additional replication or packaging signals in the DNA, a series of deletions across the entire TTMV-LY2 genome is used. Deletions of 100 bp are made stepwise across the length of the sequence. Plasmids harboring TTMV-LY2 deletions are transfected into A549 and tested as described above. In some embodiments, deletions that disrupt viral amplification or packaging will contain potential cis-regulatory domains.

Replication and packaging signals can be incorporated into effector-encoding DNA sequences (e.g., in a genetic element in a curon) to induce amplification and encapsulation. This is done both in context of larger regions of the curon genome (i.e., inserting effectors into a specific site in the genome, or replacing viral ORFs with effectors, etc.), or by incorporating minimal cis signals into the effector DNA. In cases where the curon lacks trans replication or packaging factors (e.g., replicase and capsid proteins, etc.), the trans factors are supplied by helper genes. The helper genes express all of the proteins and RNAs sufficient to induce amplification and packaging, but lack their own packaging signals. The curon DNA is co-transfected with helper genes, resulting in amplification and packaging of the effector but not of the helper genes.

### Example 9: A Minimal Anellovirus Genome

This Example describes deletions in the Anellovirus genome, both to help characterize the minimal genome sufficient for replicating virus and to insert effector payloads.

A 172-nucleotide (nt) deletion was made in the non-coding region (NCR) of TTV-tth8 downstream of the ORFs but upstream of the GC-rich region (nts 3436 to 3607). A random 56-nt sequence (TTTGTGACACAAGATGGCCGACTTCCTTCCTCTTTAGTCTTCCCCAAAGAAGACAA (SEQ ID NO: 696)) was inserted into the deletion. 2 μg of circular or linearized (by SmaI) pTTV-tth8(3436-3707::56 nt), a DNA plasmid harboring the altered TTV-tth8, was transfected into HEK293 or A549 cells at 60% confluency in a 6 cm plate using lipofectamine 2000, in duplicate. Virus was isolated from cell pellets and supernatant 96 hours post transfection by freeze thaw, alternating three times between liquid nitrogen and 37° C. water bath. Virus from supernatant was used to re-infect cells (HEK293 cells infected by virus isolated from HEK293, and A549 cells infected by virus isolated from A549). 72 hours after infection, virus was isolated from cell pellets and supernatant by freeze thaw. qPCR was performed on all samples. As shown in Table 22 below, TTV-tth8 was observed in both the cell pellet and supernatant of infected cells, indicating successful virus production by pTTV-tth8(3436-3707::56 nt).

Therefore, TTV-tth8 is able to tolerate deletion of nts 3436 to 3707.

TABLE 22
TTV-tth8(3436-3707::56nt) infections in HEK293 and A549 result in viral
amplification. Average genome equivalents from duplicate experiments
compared to negative control cells with no plasmid or virus added.

| Genome | | | | | | | |
|---|---|---|---|---|---|---|---|
| Equivalents/Rx | | HEK293 P0 | HEK293 P1 | A549 P0 | A549 P1 | Negatives | |
| TTH8 | Sup | 2.45E+06 | 1.02E+03 | 1.87E+07 | 1.00E+04 | 293 Empty | 1.42E+02 |
| Linear | Cell | 2.52E+08 | 3.92E+05 | 2.89E+08 | 7.57E+05 | 293 Neg | 5.08E+02 |
| TTH8 | Sup | 1.69E+06 | 6.83E+02 | 5.07E+02 | 1.05E+04 | 549 Empty | 1.73E+01 |
| circular | Cell | 2.00E+08 | 3.75E+05 | 2.61E+08 | 8.36E+05 | 549 Neg | 2.08E+01 |

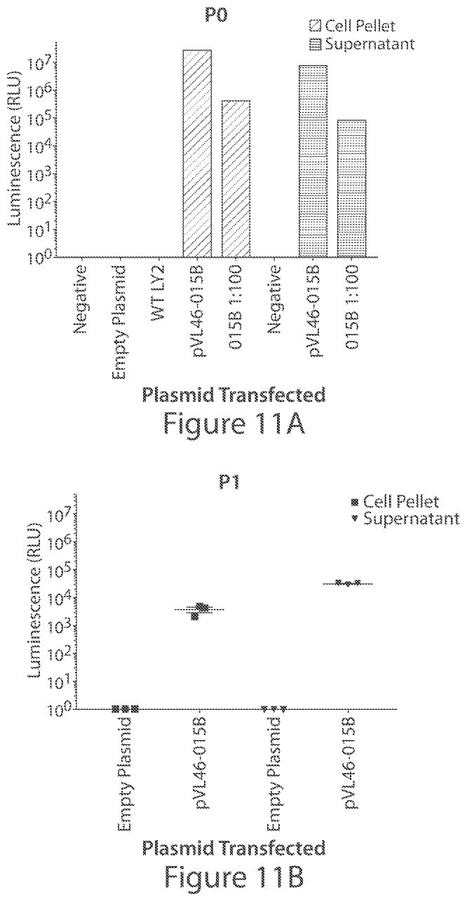

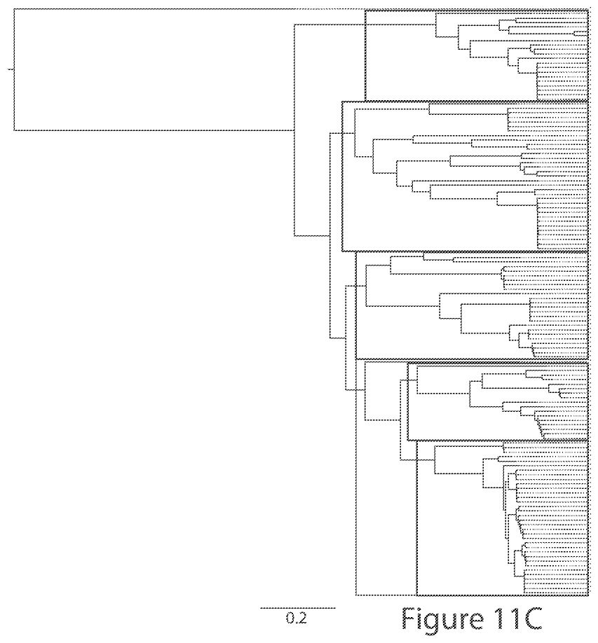

An engineered version of TTMV-LY2 was assembled, deleting nucleotides 574 to 1371 and 1432 to 2210 (1577 bp deletion) and inserting a 513 bp NanoLuc (nLuc) reporter ORF at the C-terminus of ORF1 (after nt 2609 in wild-type TTMV-LY2). Plasmids harboring the DNA sequence for the engineered TTMV-LY2 (pVL46-015B) were transfected into A549 cells, and then virus was isolated and used to infect new A549 cells, as described in Example 17. nLuc luminescence was detected in the cell pellets and supernatant of the infected cells, indicating viral replication (FIGS. 11A-11B). This demonstrates that TTMV-LY2 can tolerate at least a 1577 bp deletion in the ORF region.

To further characterize a minimal viral genome sufficient for replication, a series of deletions are made in the TTMV-LY2 DNA. A TTMV-LY2 with deletions of nts 574-1371 and 1432-2210 but no nLuc insertion is made and tested for viral replication as described previously. Further deletions are made to TTMV-LY2Δ574-1371,Δ1432-2210. Nts 1372-1431 are deleted to create TTMV-LY2Δ574-2210. Additionally, ORF3 sequence downstream of ORF1 is deleted (Δ2610-2809). Finally, to test deletions in non-coding regions, a series of 100 bp deletions are made sequentially across the NCR. All deletion mutants are tested for viral replication as previously described. Deletions that result in successful viral production (indicating that the deleted region is not essential for viral replication) are combined to make variants of TTMV-LY2 with more deleted nucleotides. This strategy will provide a minimal virus sufficient for self-amplification. To identify the minimal virus that can be amplified with helpers, each of the deletion mutants that disrupted viral replication is tested alongside helper genes carrying trans replication and packaging elements. Deletions rescued by trans expression of replication elements indicate areas of the viral genome that can be deleted to form a minimal virus when helper genes are provided from a separate source.

### Example 10: Nucleotide Insertions of Various Lengths into an Anellovirus Genome

This example describes the addition of DNA sequences of various lengths into an Anellovirus genome, which can, in some instances, be used to generate a curon as described herein.

DNA sequences are cloned into plasmids harboring TTV-tth8 (GenBank accession number AJ620231.1) and TTMV-LY2 (GenBank accession number JX134045.1). Insertions are made in the noncoding regions (NCR) 3′ of the open reading frames and 5′ of the GC-rich region: after nucleotide 3588 in TTV-tth8, or nucleotide 2843 in TTMV-LY2.

Randomized DNA sequences of the following lengths are inserted into the NCRs of TTV-tth8 and TTMV-LY2: 100 base pairs (bp), 200 bp, 500 bp, 1000 bp, and 2000 bp. These sequences are designed to match the relative GC-content of each viral genome: approximately 50% GC for insertions into TTV-tth8, and approximately 38% GC for TTMV-LY2. In addition, several trans genes are inserted into the NCR. These include a miRNA driven by a U6 promoter (351 bp) and EGFP driven by a constitutive hEF1a promoter (2509 bp).

TTV-tth8 and TTMV-LY2 variants harboring various sized DNA inserts are transfected into mammalian cell lines, including HEK293 and A549, as previously described. Virus is isolated from the supernatant or cell pellets. Isolated virus is used to infect additional cells. Production of virus from the infected cells is monitored by quantitative PCR. In some embodiments, successful production of virus will indicate tolerance of insertions.

### Example 11: Exemplary Cargo to be Delivered

This example describes exemplary classes of nucleic acid and protein payloads that may be delivered with a curon, e.g., a curon based on an Anellovirus, e.g., as described herein.

One example of a payload is mRNA for protein expression. A coding sequence of interest is transcribed from either a viral promoter native to the source virus (e.g., an Anellovirus) or from a promoter introduced with the payload as part of a trans gene. Alternatively, the mRNA is encoded within the open reading frames of the viral mRNAs, resulting in fusions between viral proteins and the protein of interest. Cleavage domains, for example, the 2A peptide or a proteinase target site, may be used to separate the protein of interest from the viral proteins when desired.

Non-coding RNAs (ncRNAs) are another example of a payload. These RNAs are generally transcribed using RNA polymerase III promoters, such as U6 or VA. Alternatively, an ncRNA is transcribed using RNA polymerase II, such as the native viral promoter or regulatable synthetic promoters. When expressed from RNA polymerase II promoters, the ncRNAs are encoded as part of the mRNA exon, introns, or as extra RNA transcribed downstream of the poly-A signal. ncRNAs are often encoded as part of a larger RNA molecule or are cleaved apart using ribozymes or endoribonucleases. ncRNAs that can be encoded as cargo in the genome of a curon include micro-RNA (miRNA), small-interfering RNAs (siRNA), short hairpin RNA (shRNA), antisense RNA, miRNA sponges, long-noncoding RNA (lncRNA), and guide RNA (gRNA).

DNA may be used as a functional element without requiring RNA transcription. For example, DNA may be used as a template for homologous recombination. In another example, a protein-binding DNA sequence may be used to drive packaging of proteins of interest into a capsid (e.g., in a proteinaceous exterior of a curon). For homologous recombination, regions of homology to human genomic DNA are encoded into the vector DNA to act as homology arms. Recombination can be driven by a targeted endonuclease (such as Cas9 with a gRNA, or a zinc-finger nuclease), which can be expressed either from the vector or from a separate source. Inside the cell, a single-stranded DNA genome is converted to double-stranded DNA, which then acts as a template for homologous recombination at the genomic DNA break site. For recruiting proteins of interest, a protein-binding sequence can be encoded in the curon DNA. A DNA-binding protein of interest, or a protein of interest fused to a DNA-binding protein (such as Gal4), binds to the curon DNA. When the curon DNA is encapsulated by the capsid proteins, the DNA-binding protein is encapsulated too, and can be delivered to cells with the curon.

### Example 12: Exemplary Payload Integration Loci

This example describes exemplary loci in the genomes of TTV-tth8 (GenBank accession number AJ620231.1) and TTMV-LY2 (GenBank accession number JX134045) into which nucleic acid payloads can be inserted.

Several strategies can be employed for insertions into the open reading frame (ORF) regions of TTV-tth8 (nucleotides 336 to 3015) and TTMV-LY2 (nucleotides 424 to 2812). In one example, in order to tag viral proteins or create fusion proteins, a payload is inserted in frame within the specific ORF of interest. Alternatively, part or all of the ORF region is deleted, which may or may not disrupt viral protein function. The payload is then inserted into the deleted region. Additionally, a hyper-variable domain (HVD) in ORF1 of TTV-tth8 (between nucleotides 716 and 2362) or TTMV-LY2 (between nucleotides 724 and 2273) can be used as an insertion site.

Alternatively, payload insertions are made into regions of the vector comparable to the non-coding regions (NCRs) of TTV-tth8 or TTMV-LY2. In particular, insertions are made in the 5′ NCR upstream of the TATA box, in the 5′ untranslated region (UTR), in the 3′ NCR downstream of the poly-A signal and upstream of the GC-rich region. Additionally, insertions are made into the miRNA region of TTV-tth8 (nucleotides 3429 to 3506). For the 5′ NCR region, insertions are made upstream of the TATA box (between nucleotides 1 and 82 in TTV-tth8, and nucleotides 1 and 236 in TTMV-LY2). In some embodiments, trans genes are inserted in the reverse orientation to reduce promoter interference. For the 5′ UTR, insertions are made downstream of the transcriptional start site (nucleotide 111 in TTV-tth8, and nucleotide 267 in TTMV-LY2) and upstream of the ORF2 start codon (nucleotide 336 in TTV-tth8, and nucleotide 421 in TTMV-LY2). 5′ UTR insertions add or replace nucleotides in the 5′ UTR. 3′ NCR insertions are made upstream of the GC-rich region, in particular after nucleotide 3588 in TTV-tth8 or nucleotide 2843 in TTMV-LY2, as described in Example 10. The miRNA of TTV-tth8 is replaced by alternative natural or synthetic miRNA hairpins.

### Example 13: Defined Categories of Anellovirus and Conserved Regions Thereof

There are three genera of Anellovirus present in humans: alphatorquevirus (Torque Teno Virus, TTV), betatorquevirus (Torque Teno Midi Virus, TTMDV), and gammatorquevirus (Torque Teno Mini Virus, TTMV). Within alphatorquevirus, there are five well-supported phylogenetic clades (FIG. 11C). It is contemplated that any of these Anelloviruses can be used as a source virus (e.g., a source of viral DNA sequences) for producing a curon as described herein.

Among these sequences, the highest conservation is found in the 5′ UTR domain (about 75% conserved) and the GC-rich domain (greater than 100 base pairs, greater than 70% GC-content, about 70% conserved). Additional, a hypervariable domain (HVD) in the sequences has very low conservation (about 30% conserved). All Anelloviruses also contain a region in which all three reading frames are open.

Also provided herein are exemplary sequences of representative viruses from each of the TTV clades, and of TTMDV and TTMV, annotated with the conserved regions (see, e.g., Tables 1-14).

### Example 14: Replication-Deficient Curons and Helper Viruses

For replication and packaging of a curon, some elements can be provided in trans. These include proteins or non-coding RNAs that direct or support DNA replication or packaging. Trans elements can, in some instances, be provided from a source alternative to the curon, such as a helper virus, plasmid, or from the cellular genome.

Other elements are typically provided in cis. These elements can be, for example, sequences or structures in the curon DNA that act as origins of replication (e.g., to allow amplification of curon DNA) or packaging signals (e.g., to bind to proteins to load the genome into the capsid). Generally, a replication deficient virus or curon will be missing one or more of these elements, such that the DNA is unable to be packaged into an infectious virion or curon even if other elements are provided in trans.

Replication deficient viruses can be useful as helper viruses, e.g., for controlling replication of a curon (e.g., a replication-deficient or packaging-deficient curon) in the same cell. In some instances, the helper virus will lack cis replication or packaging elements, but express trans elements such as proteins and non-coding RNAs. Generally, the therapeutic curon would lack some or all of these trans elements and would therefore be unable to replicate on its own, but would retain the cis elements. When co-transfected/infected into cells, the replication-deficient helper virus would drive the amplification and packaging of the curon. The packaged particles collected would thus be comprised solely of therapeutic curon, without helper virus contamination.

To develop a replication deficient curon, conserved elements in the non-coding regions of Anellovirus will be removed. In particular, deletions of the conserved 5′ UTR domain and the GC-rich domain will be tested, both separately and together. Both elements are contemplated to be important for viral replication or packaging. Additionally, deletion series will be performed across the entire non-coding region to identify previously unknown regions of interest.

Successful deletion of a replication element will result in reduction of curon DNA amplification within the cell, e.g., as measured by qPCR, but will support some infectious curon production, e.g., as monitored by assays on infected cells that can include any or all of qPCR, western blots, fluorescence assays, or luminescence assays. Successful deletion of a packaging element will not disrupt curon DNA amplification, so an increase in curon DNA will be observed in transfected cells by qPCR. However, the curon genomes will not be encapsulated, so no infectious curon production will be observed.

### Example 15: Manufacturing Process for Replication-Competent Curons

This example describes a method for recovery and scaling up of production of replication-competent curons. Curons are replication competent when they encode in their genome all the required genetic elements and ORFs necessary to replicate in cells. Since these curons are not defective in their replication they do not need a complementing activity provided in trans. They might, however need helper activity, such as enhancers of transcriptions (e.g. sodium butyrate) or viral transcription factors (e.g. adenoviral E1, E2 E4, VA; HSV Vp16 and immediate early proteins).

In this example, double-stranded DNA encoding the full sequence of a synthetic curon either in its linear or circular form is introduced into 5E+05 adherent mammalian cells in a T75 flask by chemical transfection or into 5E+05 cells in suspension by electroporation. After an optimal period of time (e.g., 3-7 days post transfection), cells and supernatant are collected by scraping cells into the supernatant medium. A mild detergent, such as a biliary salt, is added to a final concentration of 0.5% and incubated at 37° C. for 30 minutes. Calcium and Magnesium Chloride is added to a final concentration of 0.5 mM and 2.5 mM, respectively. Endonuclease (e.g. DNAse I, Benzonase), is added and incubated at 25-37° C. for 0.5-4 hours. Curon suspension is centrifuged at 1000×g for 10 minutes at 4° C. The clarified supernatant is transferred to a new tube and diluted 1:1 with a cryoprotectant buffer (also known as stabilization buffer) and stored at −80° C. if desired. This produces passage 0 of the curon (P0). To bring the concentration of detergent below the safe limit to be used on cultured cells, this inoculum is diluted at least 100-fold or more in serum-free media (SFM) depending on the curon titer.

A fresh monolayer of mammalian cells in a T225 flask is overlaid with the minimum volume sufficient to cover the culture surface and incubated for 90 minutes at 37° C. and 5% carbon dioxide with gentle rocking. The mammalian cells used for this step may or may not be the same type of cells as used for the P0 recovery. After this incubation, the inoculum is replaced with 40 ml of serum-free, animal origin-free culture medium. Cells are incubated at 37° C. and 5% carbon dioxide for 3-7 days. 4 ml of a 10× solution of the same mild detergent previously utilized is added to achieve a final detergent concentration of 0.5%, and the mixture is then incubated at 37° C. for 30 minutes with gentle agitation. Endonuclease is added and incubated at 25-37° C. for 0.5-4 hours. The medium is then collected and centrifuged at 1000×g at 4° C. for 10 minutes. The clarified supernatant is mixed with 40 ml of stabilization buffer and stored at −80° C. This generates a seed stock, or passage 1 of curon (P1).

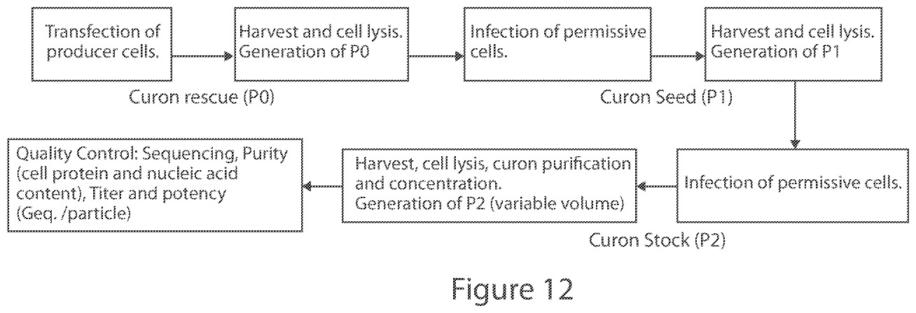

Depending on the titer of the stock, it is diluted no less than 100-fold in SFM and added to cells grown on multilayer flasks of the required size. Multiplicity of infection (MOI) and time of incubation is optimized at smaller scale to ensure maximal curon production. After harvest, curons may then be purified and concentrated as needed. A schematic showing a workflow, e.g., as described in this example, is provided in FIG. 12.

### Example 16: Manufacturing Process of Replication-Deficient Curons

This example describes a method for recovery and scaling up of production of replication-deficient curons.

Curons can be rendered replication-deficient by deletion of one or more ORFs (e.g., ORF1, ORF1/1, ORF1/2, ORF2, ORF2/2, ORF2/3, and/or ORF2t/3) involved in replication. Replication-deficient curons can be grown in a complementing cell line. Such cell line constitutively expresses components that promote curon growth but that are missing or nonfunctional in the genome of the curon.

In one example, the sequence(s) of any ORF(s) involved in curon propagation are cloned into a lentiviral expression system suitable for the generation of stable cell lines that encode a selection marker, and lentiviral vector is generated as described herein. A mammalian cell line capable of supporting curon propagation is infected with this lentiviral vector and subjected to selective pressure by the selection marker (e.g., puromycin or any other antibiotic) to select for cell populations that have stably integrated the cloned ORFs. Once this cell line is characterized and certified to complement the defect in the engineered curon, and hence to support growth and propagation of such curons, it is expanded and banked in cryogenic storage. During expansion and maintenance of these cells, the selection antibiotic is added to the culture medium to maintain the selective pressure. Once curons are introduced into these cells, the selection antibiotic may be withheld.

Once this cell line is established, growth and production of replication-deficient curons is carried out, e.g., as described in Example 15.

### Example 17: Production of Curons Using Suspension Cells

This example describes the production of curons in cells in suspension.

In this example, an A549 or 293T producer cell line that is adapted to grow in suspension conditions is grown in animal component-free and antibiotic-free suspension medium (Thermo Fisher Scientific) in WAVE bioreactor bags at 37 degrees and 5% carbon dioxide. These cells, seeded at 1×10⁶ viable cells/mL, are transfected using lipofectamine 2000 (Thermo Fisher Scientific) under current good manufacturing practices (cGMP), with a plasmid comprising curon sequences, along with any complementing plasmids suitable or required to package the curon (e.g., in the case of a replication-deficient curon, e.g., as described in Example 16). The complementing plasmids can, in some instances, encode for viral proteins that have been deleted from the curon genome (e.g., a curon genome based on a viral genome, e.g., an Anellovirus genome, e.g., as described herein) but are useful or required for replication and packaging of the curons. Transfected cells are grown in the WAVE bioreactor bags and the supernatant is harvested at the following time points: 48, 72, and 96 hours post transfection. The supernatant is separated from the cell pellets for each sample using centrifugation. The packaged curon particles are then purified from the harvested supernatant and the lysed cell pellets using ion exchange chromatography.

The genome equivalents in the purified prep of the curons can be determined, for example, by using a small aliquot of the purified prep to harvest the curon genome using a viral genome extraction kit (Qiagen), followed by qPCR using primers and probes targeted towards the curon DNA sequence, e.g., as described in Example 18.

The infectivity of the curons in the purified prep can be quantified by making serial dilutions of the purified prep to infect new A549 cells. These cells are harvested 72 hours post transfection, followed by a qPCR assay on the genomic DNA using primers and probes that are specific to the curon DNA sequence.

### Example 18: Quantification of Curon Genome Equivalents by qPCR

This example demonstrates the development of a hydrolysis probe-based quantitative PCR assay to quantify curons. Sets of primers and probes were designed based on selected genome sequences of TTV (Accession No. AJ620231.1) and TTMV (Accession No. JX134045.1) using the software Geneious with a final user optimization. Primer sequences are shown in Table 23 below.

TABLE 23
Sequences of forward and reverse primers and hydrolysis probes
used to quantify TTMV and TTV genome equivalents by quantitative PCR.

| TTMV | | SEQ ID NO: |
|---|---|---|
| Forward Primer | 5′-GAAGCCCACCAAAAGCAATT-3′ | 697 |
| Reverse Primer | 5′-AGTTCCCGTGTCTATAGTCGA-3′ | 698 |
| Probe | 5′-ACTTCGTTACAGAGTCCAGGGG-3′ | 699 |
| TTV | | |
| Forward Primer | 5′-AGCAACAGGTAATGGAGGAC-3′ | 700 |
| Reverse Primer | 5′-TGGAAGCTGGGGTCTTTAAC-3′ | 701 |
| Probe | 5′-TCTACCTTAGGTGCAAAGGGCC-3′ | 702 |

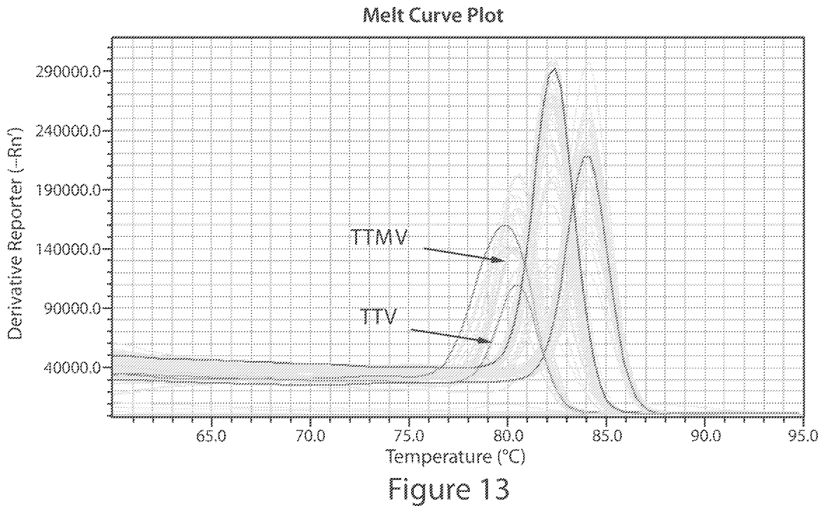

As a first step in the development process, qPCR is run using the TTV and TTMV primers with SYBR-green chemistry to check for primer specificity. FIG. 13 shows one distinct amplification peak for each primer pair.

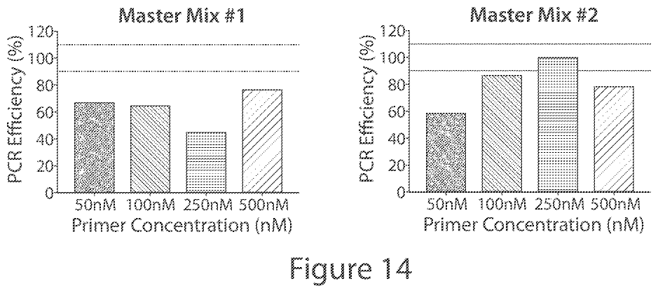

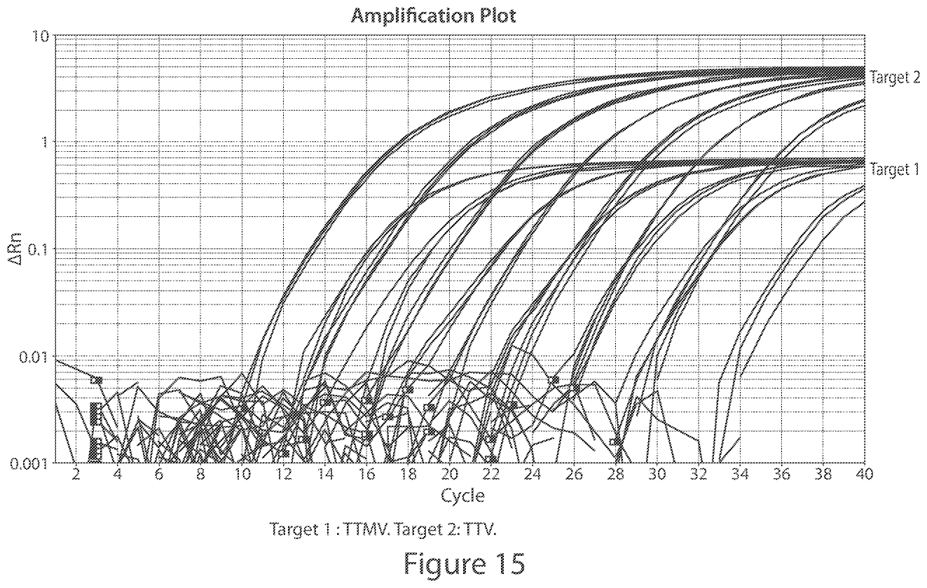

Hydrolysis probes were ordered labeled with the fluorophore 6FAM at the 5′ end and a minor groove binding, non-fluorescent quencher (MGBNFQ) at the 3′ end. The PCR efficiency of the new primers and probes was then evaluated using two different commercial master mixes using purified plasmid DNA as component of a standard curve and increasing concentrations of primers. The standard curve was set up by using purified plasmids containing the target sequences for the different sets of primers-probes. Seven tenfold serial dilutions were performed to achieve a linear range over 7 logs and a lower limit of quantification of 15 copies per 20 ul reaction. Master mix #2 was capable of generating a PCR efficiency between 90-110%, values that are acceptable for quantitative PCR (FIG. 14). All primers for qPCR were ordered from IDT. Hydrolysis probes conjugated to the fluorophore 6FAM and a minor groove binding, non-fluorescent quencher (MGBNFQ) as well as all the qPCR master mixes were obtained from Thermo Fisher. An exemplary amplification plot is shown in FIG. 15.

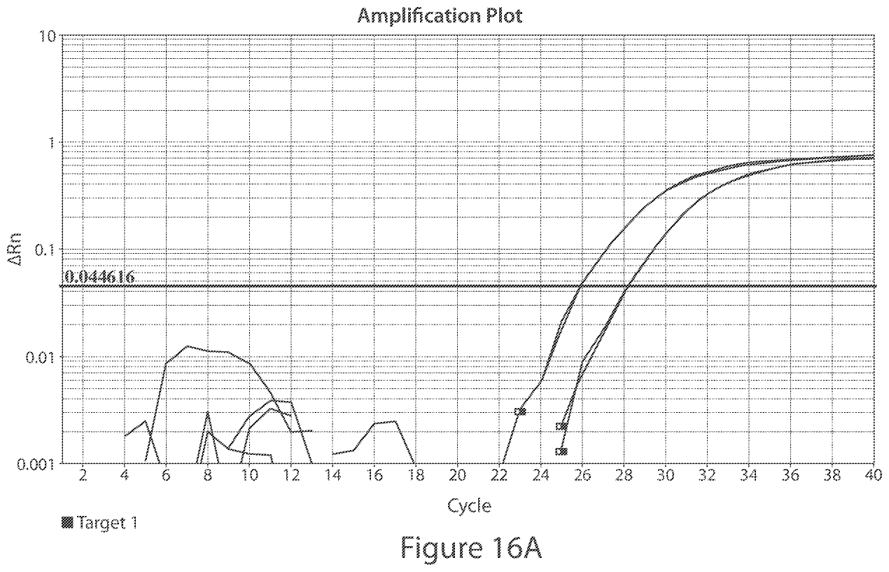

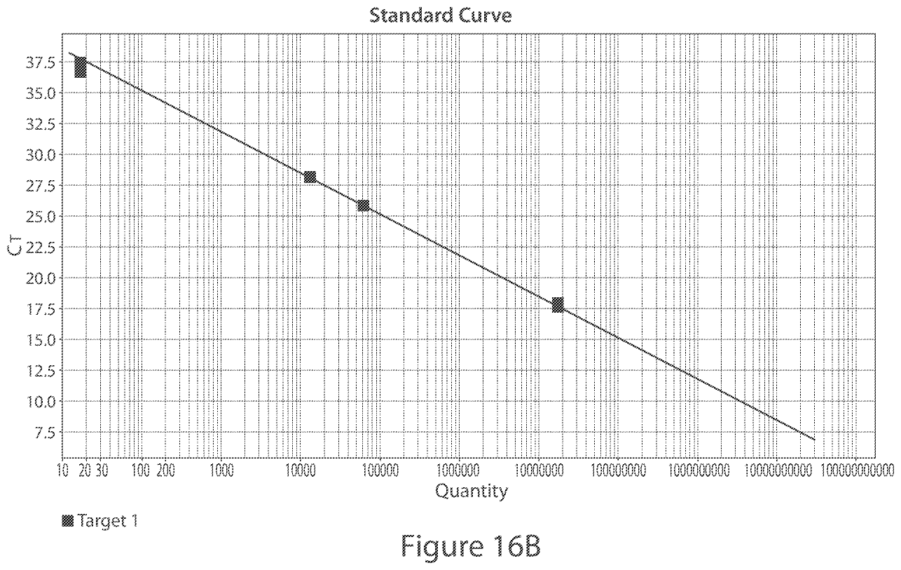

Using these primer-probe sets and reagents, the genome equivalent (GEq)/ml in curon stocks was quantified. The linear range was between 1.5E+07-15 GEq per 20 ul reaction, which was then used to calculate the GEq/ml, as shown in FIGS. 16A-16B. Samples with higher concentrations than the linear range can be diluted as needed.

### Example 19: Utilizing Curons to Express an Exogenous Protein in Mice

This example describes the usage of a curon in which the Torque Teno Mini Virus (TTMV) genome is engineered to express the firefly luciferase protein in mice.

The plasmid encoding the DNA sequence of the engineered TTMV encoding the firefly-luciferase gene is introduced into A549 cells (human lung carcinoma cell line) by chemical transfection. 18 ug of plasmid DNA is used for transfection of 70% confluent cells in a 10 cm tissue culture plate. Empty vector backbone lacking the TTMV sequences is used as a negative control. Five hours post-transfection, cells are washed with PBS twice and are allowed to grow in fresh growth medium at 37° C. and 5% carbon dioxide.

Transfected A549 cells, along with their supernatant, are harvested 96 hours post transfection. Harvested material is treated with 0.5% deoxycholate (weight in volume) at 37° C. for 1 hour followed by endonuclease treatment. Curon particles are purified from this lysate using ion exchange chromatography. To determine curon concentration, a sample of the curon stock is run through a viral DNA purification kit and genome equivalents per ml are measured by qPCR using primers and probes targeted towards the curon DNA sequence.

A dose-range of genome equivalents of curons in 1× phosphate-buffered saline is performed via a variety of routes of injection (e.g. intravenous, intraperitoneal, subcutaneous, intramuscular) in mice at 8-10 weeks of age. Ventral and dorsal bioluminescence imaging is performed on each animal at 3, 7, 10 and 15 days post injection. Imaging is performed by adding the luciferase substrate (Perkin-Elmer) to each animal intraperitoneally at indicated time points, according to the manufacturer's protocol, followed by intravital imaging.

### Example 20: Genome Alignments to Determine Whether Curon DNA Integrated into Host Genomes

This example describes the computational analysis performed to determine whether curon DNA can integrate into the host genome, by examining whether Torque Teno Virus (TTV) has integrated into the human genome.

The complete genomes of one representative TTV sequence from each of clades 1-5 were aligned against the human genome sequence using the Basic Local Alignment Search Tool (BLAST) that finds regions of local similarity between sequences. The representative TTV sequences shown in Table 24 were analyzed:

TABLE 24
Representative TTV sequences

| TTV Clade | NCBI Accession No. |
|---|---|
| Clade 1 | AB064597.1 |
| Clade 2 | AB028669.1 |
| Clade 3 | AJ20231.1 |
| Clade 4 | AF122914.3 |
| Clade 5 | AF298585.1 |

Sequences from none of the aligned TTVs were found to have any significant similarity to the human genome, indicating that the TTVs have not integrated into the human genome.

### Example 21: Assessment of Curon Integration into a Host Genome

In this example, A549 cells (human lung carcinoma cell line) and HEK293T cells (human embryonic kidney cell line) are infected with either curon particles or AAV particles at MOIs of 5, 10, 30 or 50. The cells are washed with PBS 5 hours post infection and replaced with fresh growth medium. The cells are then allowed to grow at 37 degrees and 5% carbon dioxide. Cells are harvested five days post infection and they are processed to harvest genomic DNA, using the genomic DNA extraction kit (Qiagen). Genomic DNA is also harvested from uninfected cells (negative control). Whole-genome sequencing libraries are prepared for these harvested DNAs, using the Nextera DNA library preparation kit (Illumina), according to manufacturers protocol. The DNA libraries are sequenced using the NextSeq 550 system (Illumina) according to manufacturer's protocol. Sequencing data is assembled to the reference genome and analyzed to look for junctions between curon or AAV genomes and host genome. In cases where junctions are detected they are verified in the original genomic DNA sample prior sequencing library preparation by PCR. Primers are designed to amplify the region containing and around the junctions. The frequency of integration of Curons into the host genome is determined by quantifying the number of junctions (representing integration events) and the total number of curon copies in the sample by qPCR. This ratio can be compared to that of AAV.

### Example 22: Functional Effects of a Curon Expressing an Exogenous microRNA Sequence

This example provides a successful demonstration of function of curons expressing exogenous microRNA (miRNA) sequences.

Curon DNA sequences were generated that contained one of the following exogenous microRNA sequences in the 3′ non-coding region (NCR):
1) miR-124
2) miR-518
3) miR-625
4) Non-targeting scramble miRNA (miR-scr)

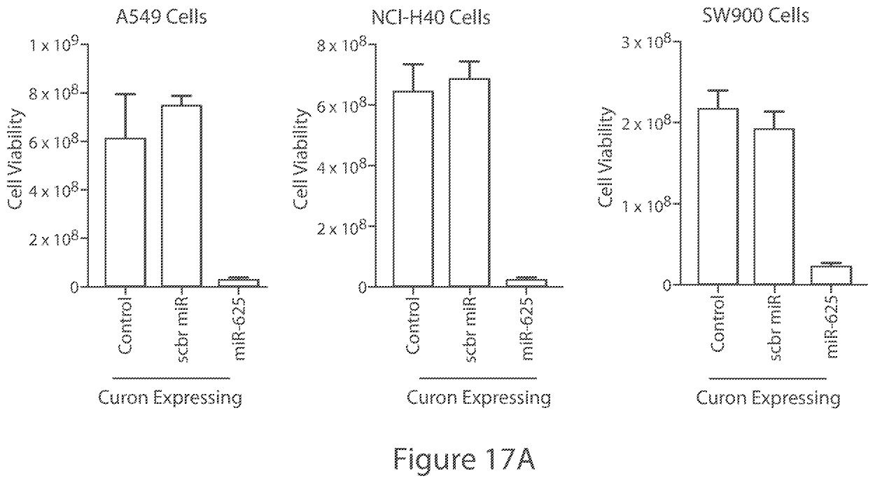

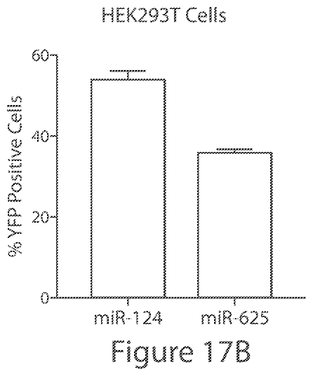

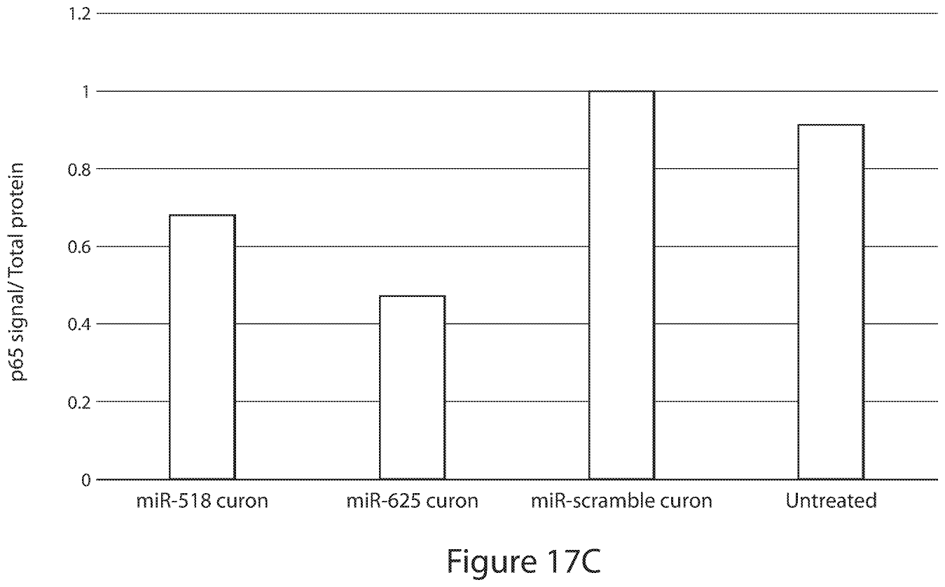

This was done by replacing the pre-miRNA sequence of the tth8-T1 miRNA of TTV-tth8 with the pre-miRNA sequences of the miRNAs mentioned above. Curon DNAs were then transfected into HEK293T cells seperately. Transfected 293T cells, along with the supernatants were harvested 96 hours post transfection. Harvested material was treated with 0.5% deoxycholate (weight in volume) at 37 degrees Celsius, followed by endonuclease treatment. This lysate containing the packaged curons (P0 stock of curons) were used to infect new 293T cells. These cells were harvested 96 hours, post infection. The harvested cells were then treated with 0.5% deoxycholate (weight in volume) at 37 degrees Celsius, followed by endonuclease treatment. This lysate was then dialyzed in the 10K molecular-weight cutoff dialysis cassettes in PBS at 4 degrees overnight to remove any deoxycholate. The titer of the curon was quantified in these dialyzed lysate (P1 stock of curon) using qPCR. P1 stock of curons were then incubated with several KRAS mutant non-small cell lung cancer (NSCLC) cell lines (SW900, NCI-H460, and A549) for 3 days at a titer of 274 genome equivalents per cell. Cell viability was measured with an Alamar blue assay. As shown in FIG. 17A, curons expressing an exogenous miR-625 significantly inhibited cancer cell line viability in all three NSCLC cell lines as compared to cells infected with control curons expressing a scrambled non-targeted miRNA and uninfected cells.

Additionally, a YFP-reporter assay was used to determine the downregulation of the target by curon miRNA by site specific binding to its target site. A YFP reporter that has a specific binding sequence for miR-625 was generated and transfected into HEK293T cells. 24 hours after transfection, these HEK293T cells were infected with curons expressing either miR-625 or a non-specific miRNA (miR-124) at a titer of 2.4 genome equivalents per cell, and YFP fluorescence was then measured using flow cytometry. As shown in FIG. 17B, curons expressing miR-625 significantly downregulated YFP expression, whereas curons expressing the non-specific miRNA miR-124 did not affect YFP expression. These results show that the curon with miR-625 induced on-target downregulation of the YFP protein target.

The ability of curons expressing exogenous miRNAs to modulate host gene expression was also tested. SW-900 NSCLC cells were infected with Curons expressing either miR-518 or miR-625 or miR-scr at a dose of 10 genome equivalents per cell. Infected cells were harvested 72 hours post infection and total protein lysates were prepared. Immunoblot analysis was performed on these protein lysates to determine the levels of p65 protein. The intensity of p65 protein signal was normalized to the total amount of protein on the membrane for each sample (FIG. 17C). A reduction in p65 levels was observed, indicating that curons can modulate expression of a host gene.

### Example 23: Preparation and Production of Curons to Express Exogenous Non-Coding RNAs

This example describes the synthesis and production of curons to express exogenous small non-coding RNAs.

The DNA sequence from the tth8 strain of TTV (Jelcic et al, Journal of Virology, 2004) is synthesized and cloned into a vector containing the bacterial origin of replication and bacterial antibiotic resistance gene. In this vector, the DNA sequence encoding the TTV miRNA hairpin is replaced by a DNA sequence encoding an exogenous small non-coding RNA such as miRNA or shRNA. The engineered construct is then transformed into electro-competent bacteria, followed by plasmid isolation using a plasmid purification kit according to the manufacturer's protocols.

The curon DNA encoding the exogenous small non-coding RNAs is transfected into an eukaryotic producer cell line to produce curon particles. The supernatant of the transfected cells containing the curon particles is harvested at different time points post transfection. Curon particles, either from the filtered supernatant or after purification, are used for downstream applications, e.g., as described herein.

### Example 24: Conservation in Anellovirus Clades

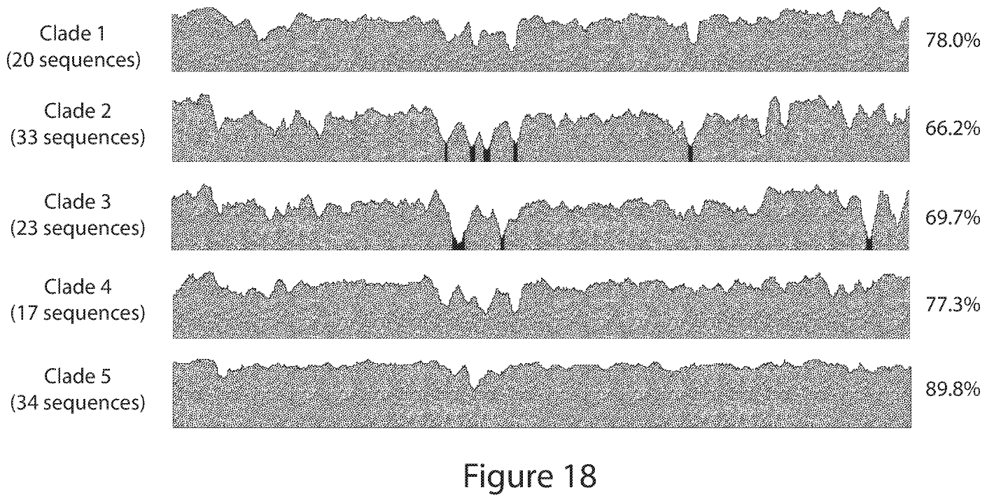

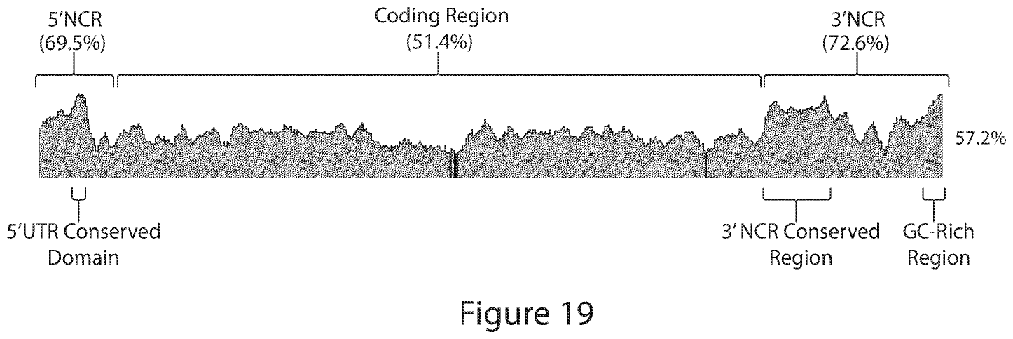

This example describes the identification of five clades within the alphatorquevirus genus. The average pairwise identity within each clade generally ranges from 66 to 90% (FIG. 18). Representative sequences between these clades showed 57.2% pairwise identity across the sequences (FIG. 19). The pairwise identity is lowest among the open reading frames (˜51.4%), and higher in the non-coding regions (69.5% in the 5′ NCR, 72.6% in the 3′ NCR) (FIG. 19). This suggests that DNA sequences or structures in the non-coding regions play important roles in viral replication.

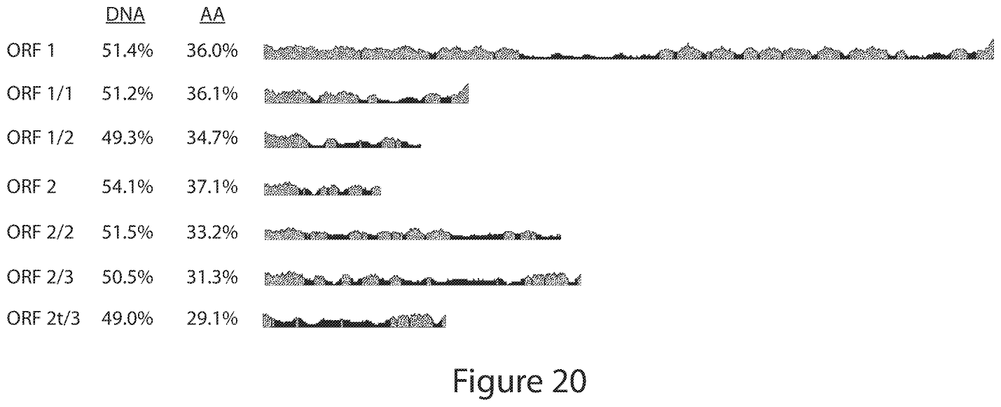

The amino acid sequences of the putative proteins in alphatorquevirus were also compared. The DNA sequences showed approximately 49 to 54% pairwise identity, while the amino acid sequences showed approximately 29 to 36% pairwise identity (FIG. 20). Interestingly, the representative sequences from the alphatorquevirus clades are able to successfully replicate in vivo and are observed in the human population. This suggests that the amino acid sequences for anellovirus proteins can vary widely while retaining functionalities such as replication and packaging.

Anelloviruses were found to have regions of local high conservation in the non-coding regions. In the region downstream of the promoter is a 71-bp 5′ UTR conserved domain that has 96.6% pairwise identity across the five alphatorquevirus clades (FIG. 21). Downstream of the open reading frames in the 3′ non-coding region of alphatorqueviruses, there is a 307 bp region with 85.2% pairwise identity between the representative sequences (FIG. 19). Near the 3′ end of this 3′ conserved non-coding region is a highly conserved 51 bp sequence with 96.5% pairwise identity. Each Anellovirus studied in this analysis also includes a GC-rich region, with greater than 70% GC content (FIG. 22).

### Example 25: Expression of an Endogenous miRNA from a Curon and Deletion of the Endogenous miRNA

In one example, curons based on the TTV-tth8 strain were used to infect Raji B cells in culture. These curons comprised a sequence encoding the endogenous payload of the TTV-tth8 Anellovirus, which is a miRNA targeting the mRNA encoding n-myc interacting protein (NMI). NMI operates downstream of the JAK/STAT pathway to regulate the transcription of various intracellular signals, including interferon-stimulated genes, proliferation and growth genes, and mediators of the inflammatory response. As shown in FIG. 23A, curons were able to successfully infect Raji B cells. Infection of cells with curons comprising the miRNA against NMI resulted in successful knockdown of NMI compared to control cells infected with curons lacking the miRNA against NMI (FIG. 23B). Cells infected with curon comprising the miRNA against NMI showed a greater than 75% reduction in NMI protein levels compared to control cells. This example demonstrates that a curon with a native Anellovirus miRNA can knock down a target molecule in host cells.

In another example, the endogenous miRNA of an Anellovirus-based curon was deleted. The resultant curon (ΔmiR) was then used to infect host cells. Infection rate was compared to that of corresponding curons in which the endogenous miRNA was retained. As shown in FIG. 24, curons in which the endogenous miRNA were deleted were still able to infect cells at levels comparable to those observed for curons in which the endogenous miRNA was still present. This example demonstrates that the endogenous miRNA of an Anellovirus-based curon can be mutated, or deleted entirely, and still generate infectious particles.

What is claimed is:

1. A synthetic curon comprising:
(i) a genetic element comprising a promoter element and a nucleic acid sequence encoding an exogenous effector, and a protein binding sequence, wherein the genetic element comprises one or both of:
(a) a sequence having at least 85% sequence identity to the Anellovirus 5′ UTR conserved domain nucleotide sequence of nucleotides 323-393 of the nucleic acid sequence of Table 11, or
(b) a sequence having at least 85% sequence identity to the Anellovirus GC-rich region of nucleotides 2868-2929 of the nucleic acid sequence of Table 11; and
(ii) a proteinaceous exterior; wherein the genetic element is enclosed within the proteinaceous exterior; and
wherein the synthetic curon is capable of delivering the genetic element into a eukaryotic cell.

2. The synthetic curon of claim 1, wherein the genetic element is single-stranded.

3. The synthetic curon of any of the preceding claims, wherein the genetic element is DNA.

4. The synthetic curon of claim 3, wherein the genetic element is a negative strand DNA.

5. The synthetic curon of any of the preceding claims, wherein the genetic element integrates at a frequency of less than 10%, 8%, 6%, 4%, 3%, 2%, 1%, 0.5%, 0.2%, 0.1% of the curons that enters the cell, e.g., wherein the synthetic curon is non-integrating.

6. The synthetic curon of any of the preceding claims, wherein the genetic element comprises a sequence of the Consensus 5′ UTR nucleic acid sequence shown in Table 16-1.

7. The synthetic curon of any of the preceding claims, wherein the genetic element comprises a sequence of the Consensus GC-rich region shown in Table 16-2.

8. The synthetic curon of any of the preceding claims, wherein the genetic element comprises a sequence of at least 100 nucleotides in length, which consists of G or C at least 70% (e.g., about 70-100%, 75-95%, 80-95%, 85-95%, or 85-90%) of the positions.

9. The synthetic curon of any of the preceding claims, wherein the genetic element comprises a sequence having at least 85% sequence identity to the Anellovirus 5′ UTR conserved domain nucleotide sequence of nucleotides 1-393 of the nucleic acid sequence of Table 11 and a sequence having at least 85% sequence identity to the Anellovirus GC-rich region of nucleotides 2868-2929 of the nucleic acid sequence of Table 11.

10. The synthetic curon of any of the preceding claims, wherein the genetic element comprises at least 75% identity to the nucleotide sequence of Table 11.

11. The synthetic curon of any of the preceding claims, wherein the promoter element is exogenous to wild-type Anellovirus.

12. The synthetic curon of any of claims 1-10, wherein the promoter element is endogenous to wild-type Anellovirus.

13. The synthetic curon of any of the preceding claims, wherein the exogenous effector encodes a therapeutic agent, e.g., a therapeutic peptide or polypeptide or a therapeutic nucleic acid.

14. The synthetic curon of any of the preceding claims, wherein the exogenous effector comprises a regulatory nucleic acid, e.g., an miRNA, siRNA, mRNA, lncRNA, RNA, DNA, an antisense RNA, gRNA; a fluorescent tag or marker, an antigen, a peptide, a synthetic or analog peptide from a naturally-bioactive peptide, an agonist or antagonist peptide, an anti-microbial peptide, a pore-forming peptide, a bicyclic peptide, a targeting or cytotoxic peptide, a degradation or self-destruction peptide, a small molecule, an immune effector (e.g., influences susceptibility to an immune response/signal), a death protein (e.g., an inducer of apoptosis or necrosis), a non-lytic inhibitor of a tumor (e.g., an inhibitor of an oncoprotein), an epigenetic modifying agent, an epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand, an antibody, a receptor, or a CRISPR system or component.

15. The synthetic curon of any of the preceding claims, wherein the exogenous effector comprises an miRNA, and decreases expression of a host gene.

16. The synthetic curon of any of the preceding claims, wherein the exogenous effector comprises a nucleic acid sequence about 20-200, 30-180, 40-160, 50-140, or 60-120 nucleotides in length.

17. The synthetic curon of any of the preceding claims, wherein the nucleic acid sequence encoding the exogenous effector is about 20-200, 30-180, 40-160, 50-140, or 60-120 nucleotides in length.

18. The synthetic curon of any of the preceding claims, wherein the sequence encoding the exogenous effector is situated at, within, or adjacent to (e.g., 5′ or 3′ to) one or more of the ORF1 locus, e.g., at the C-terminus of the ORF1 locus, or the 3′ noncoding region downstream of the poly-A region.

19. The synthetic curon of any of the preceding claims, wherein the sequence encoding the exogenous effector is located between the poly-A region and the GC-rich region of the genetic element.

20. The synthetic curon of any of the preceding claims, which comprises (e.g., in the proteinaceous exterior) one or more of an amino acid sequence chosen from ORF2, ORF2/2, ORF2/3, ORF1, ORF1/1, or ORF1/2 of Table 12, or an amino acid sequence having at least 85% sequence identity thereto.

21. The synthetic curon of any of the preceding claims, wherein the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

22. The synthetic curon of any of the preceding claims, wherein the synthetic curon does not comprise a lipid bilayer.

23. The synthetic curon of any of the preceding claims, wherein the synthetic curon is capable of infecting mammalian cells, e.g., human cells, e.g., immune cells, liver cells, or lung epithelial cells.

24. The synthetic curon of any of the preceding claims, wherein the genetic element is capable of replicating, e.g., capable of generating at least 10², 2×10², 5×10², 10³, 2×10³, 5×10³, or 10⁴ genomic equivalents of the genetic element per cell, e.g., as measured by a quantitative PCR assay.

25. The synthetic curon of any of the preceding claims, which is substantially non-pathogenic, e.g., does not induce a detectable deleterious symptom in a subject (e.g., elevated cell death or toxicity, e.g., relative to a subject not exposed to the curon).

26. The synthetic curon of any of the preceding claims, which is substantially non-immunogenic, e.g., does not induce a detectable and/or unwanted immune response, e.g., as detected according to the method described in Example 4.

27. The synthetic curon of claim 26, wherein the substantially non-immunogenic curon has an efficacy in a subject that is a least about 10%, 20%, 30%, 40%, 50%, 60%, 70%, 80%, 90%, 95%, or 100% of the efficacy in a reference subject lacking an immune response.

28. The synthetic curon of claim 26 or 27, wherein the immune response comprises one or more of an antibody specific to the curon; a cellular response (e.g., an immune effector cell (e.g., T cell- or NK cell) response) against the curon or cells comprising the curon; or macrophage engulfment of the curon or cells comprising the curon.

29. The synthetic curon of any of the preceding claims, wherein a population of at least 1000 of the synthetic curons is capable of delivering at least 100 copies of the genetic element into one or more of the eukaryotic cells.

30. A synthetic curon comprising:
(i) a genetic element comprising a promoter element and a nucleic acid sequence encoding an exogenous effector, and a protein binding sequence, wherein the genetic element comprises one or both of:
(a) a sequence having at least 85% sequence identity to the Anellovirus 5′ UTR conserved domain of the nucleic acid sequence of Table 1, 3, 5, 7, 9 or 13; or
(b) a sequence having at least 85% sequence identity to the Anellovirus GC-rich region of the nucleic acid sequence of Table 1, 3, 5, 7, 9 or 13;
and
(ii) a proteinaceous exterior; wherein the genetic element is enclosed within the proteinaceous exterior; and
wherein the synthetic curon is capable of delivering the genetic element into a eukaryotic cell.

31. The synthetic curon of claim 30, which comprises (e.g., in the proteinaceous exterior) one or more of an amino acid sequence chosen from ORF2, ORF2/2, ORF2/3, ORF2t/3, ORF1, ORF1/1, or ORF1/2 of any of Tables 2, 4, 6, 8, 10, or 14, or an amino acid sequence having at least 85% sequence identity thereto.

32. A nucleic acid molecule comprising a promoter element and a nucleic acid sequence encoding an exogenous effector, and a protein binding sequence, wherein the genetic element comprises one or both of:
(a) a sequence having at least 85% sequence identity to the Anellovirus 5′ UTR conserved domain nucleotide sequence of nucleotides 323-393 of the nucleic acid sequence of Table 11, or
(b) a sequence having at least 85% sequence identity to the Anellovirus GC-rich region of nucleotides 2868-2929 of the nucleic acid sequence of Table 11.

33. A nucleic acid molecule comprising a promoter element and a nucleic acid sequence encoding an exogenous effector, and a protein binding sequence, wherein the genetic element comprises one or both of:
(a) a sequence having at least 85% sequence identity to the Anellovirus 5′ UTR conserved domain nucleotide sequence of the nucleic acid sequence of Table 1, 3, 5, 7, or 13, or
(b) a sequence having at least 85% sequence identity to the Anellovirus GC-rich region of the nucleic acid sequence of Table 1, 3, 5, 7, or 13.

34. A pharmaceutical composition comprising the synthetic curon of any of the preceding claims, and a pharmaceutically acceptable carrier or excipient.

35. The pharmaceutical composition of claim 34, which comprises at least 10³, 10⁴, 10⁵, 10⁶, 10⁷, 10⁸, or 10⁹ synthetic curons.

36. A reaction mixture comprising the synthetic curon of any of claims 1-31 and a second nucleic acid sequence encoding one or more of an amino acid sequence chosen from ORF2, ORF2/2, ORF2/3, ORF1, ORF1/1, or ORF1/2 of Table 12, or an amino acid sequence having at least 85% sequence identity thereto.

37. A reaction mixture comprising the synthetic curon of any of claims 1-31 and a second nucleic acid sequence encoding one or more of an amino acid sequence chosen from ORF2, ORF2/2, ORF2/3, ORF2t/3, ORF1, ORF1/1, or ORF1/2 of any of Tables 2, 4, 6, 8, 10, or 14, or an amino acid sequence having at least 85% sequence identity thereto.

38. The reaction mixture of claim 36 or 37, wherein the second nucleic acid sequence is part of the genetic element.

39. The reaction mixture of claim 36 or 37, wherein the second nucleic acid sequence is not part of the genetic element, e.g., the second nucleic acid sequence is comprised by a helper cell or helper virus.

40. Use of a synthetic curon of any of the claims 1-31 or the pharmaceutical composition of any of claims 34-35 for delivering the genetic element to a host cell.

41. Use of a synthetic curon of any of the claims 1-31 or the pharmaceutical composition of any of claims 34-35 for treating a disease or disorder in a subject.

42. The use of claim 41, wherein the disease or disorder is chosen from an immune disorder, an interferonopathies (e.g., Type I interferonopathy), infectious disease, inflammatory disorder, autoimmune condition, cancer (e.g., a solid tumor, e.g., lung cancer), and a gastrointestinal disorder.

43. A synthetic curon of any of claims 1-31 or the pharmaceutical composition of any of claims 34-35, for use in treating a disease or disorder in a subject.

44. A method of treating a disease or disorder in a subject, the method comprising administering a synthetic curon of any of claims 1-31 or the pharmaceutical composition of any of claims 34-35 to the subject, wherein the disease or disorder is chosen from an immune disorder, an interferonopathy (e.g., Type I interferonopathy), infectious disease, inflammatory disorder, autoimmune condition, cancer (e.g., a solid tumor, e.g., lung cancer), and a gastrointestinal disorder.

45. A method of manufacturing a synthetic curon composition, comprising:
a) providing a plurality of synthetic curons according to claims 1-31, or a composition or pharmaceutical composition of any of claims 34-35;
b) optionally evaluating the plurality for one or more of: a contaminant described herein, an optical density measurement (e.g., OD 260), particle number (e.g., by HPLC), infectivity (e.g., particle:infectious unit ratio); and
c) formulating the plurality of synthetic curons, e.g., as a pharmaceutical composition suitable for administration to a subject, e.g., if one or more of the parameters of (b) meet a specified threshold.
